(19) United States
(12) Patent Application Publication
(10) Pub. No.: US 2020/0055839 A1
(43) Pub. Date: Feb. 20, 2020

(54) PYRIDIN-3-YL ACETIC ACID DERIVATIVES AS INHIBITORS OF HUMAN IMMUNODEFICIENCY VIRUS REPLICATION

(71) Applicant: VIIV HEALTHCARE UK (NO.5) LIMITED, Brentford, Middlesex (GB)

(72) Inventors: Makonen BELEMA, Wallingford, CT (US); Michael S. BOWSHER, Wallingford, CT (US); Jeffrey A. DESKUS, Wallingford, CT (US); Kyle J. EASTMAN, Wallingford, CT (US); Eric P. GILLIS, Wallingford, CT (US); David B. FRENNESSON, Wallingford, CT (US); Christiana IWUAGWU, Wallingford, CT (US); John F. KADOW, Branford, CT (US); B. Narasimhulu NAIDU, Branford, CT (US); Kyle E. PARCELLA, Branford, CT (US); Kevin M. PEESE, Branford, CT (US); Mark G. SAULNIER, Wallingford, CT (US); Prasanna SIVAPRAKASAM, Wallingford, CT (US)

(21) Appl. No.: 16/465,199

(22) PCT Filed: Jan. 2, 2018
(86) PCT No.: PCT/IB2018/050021

§ 371 Date: May 30, 2019

Related U.S. Application Data
(60) Provisional application No. 62/588,628, filed on Nov. 20, 2017.
(60) Provisional application No. 62/441,612, filed on Jan. 3, 2017.

(51) Int. Cl. C07D 401/14; C07D 401/04; C07D 491/048; C07D 405/14; C07D 413/14; A61P 31/18

(52) U.S. Cl. CPC C07D 401/14; C07D 401/04; C07D 491/048; A61K 45/06; C07D 413/14; A61P 31/18; C07D 405/14

(57) ABSTRACT

Disclosed are compounds of Formula I, including pharmaceutically acceptable salts, pharmaceutical compositions comprising the compounds, methods for making the compounds and their use in inhibiting HIV integrase and treating those infected with HIV or AIDS.

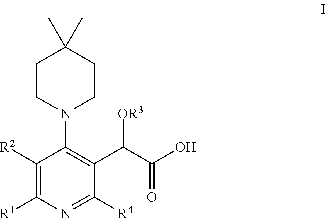

### FIELD OF THE INVENTION

The invention relates to compounds, compositions, and methods for the treatment of human immunodeficiency virus (HIV) infection. More particularly, the invention provides novel inhibitors of HIV, pharmaceutical compositions containing such compounds, and methods for using these compounds in the treatment of HIV infection. The invention also relates to methods for making the compounds hereinafter described.

### BACKGROUND OF THE INVENTION

Human immunodeficiency virus (HIV) has been identified as the etiological agent responsible for acquired immune deficiency syndrome (AIDS), a fatal disease characterized by destruction of the immune system and the inability to fight off life threatening opportunistic infections. Recent statistics indicate that an estimated 35.3 million people worldwide are infected with the virus (UNAIDS: Report on the Global HIV/AIDS Epidemic, 2013). In addition to the large number of individuals already infected, the virus continues to spread. Estimates from 2013 point to close to 3.4 million new infections in that year alone. In the same year there were approximately 1.6 million deaths associated with HIV and AIDS.

Current therapy for HIV-infected individuals consists of a combination of approved anti-retroviral agents. Over two dozen drugs are currently approved for HIV infection, either as single agents or as fixed dose combinations or single tablet regimens, the latter two containing 2-4 approved agents. These agents belong to a number of different classes, targeting either a viral enzyme or the function of a viral protein during the virus replication cycle. Thus, agents are classified as either nucleotide reverse transcriptase inhibitors (NRTIs), non-nucleotide reverse transcriptase inhibitors (NNRTIs), protease inhibitors (PIs), integrase inhibitors (INIs), or entry inhibitors (one, maraviroc, targets the host CCR5 protein, while the other, enfuvirtide, is a peptide that targets the gp41 region of the viral gp160 protein). In addition, a pharmacokinetic enhancer with no antiviral activity, i.e., cobicistat, available from Gilead Sciences, Inc. under the tradename TYBOST™ (cobicistat) tablets, has recently been approved for use in combinations with certain antiretroviral agents (ARVs) that may benefit from boosting.

In the US, where combination therapy is widely available, the number of HIV-related deaths has dramatically declined (Palella, F. J.; Delany, K. M.; Moorman, A. C.; Loveless, M. O.; Furher, J.; Satten, G. A.; Aschman, D. J.; Holmberg, S. D. N. Engl. J. Med. 1998, 338, 853-860).

Unfortunately, not all patients are responsive and a large number fail this therapy. In fact, initial studies suggest that approximately 30-50% of patients ultimately fail at least one drug in the suppressive combination. Treatment failure in most cases is caused by the emergence of viral resistance. Viral resistance in turn is caused by the replication rate of HIV-1 during the course of infection combined with the relatively high viral mutation rate associated with the viral polymerase and the lack of adherence of HIV-infected individuals in taking their prescribed medications. Clearly, there is a need for new antiviral agents, preferably with activity against viruses already resistant to currently approved drugs. Other important factors include improved safety and a more convenient dosing regimen than many of the currently approved drugs.

Compounds which inhibit HIV replication have been disclosed. See, for example, the following patent applications: WO2007131350, WO2009062285, WO2009062288, WO2009062289, WO2009062308, WO2010130034, WO2010130842, WO2011015641, WO2011076765, WO2012033735, WO2013123148, WO2013134113, WO2014164467, WO2014159959, WO2015126726, and WO2017025915.

What is now needed in the art are additional compounds which are novel and useful in the treatment of HIV. Additionally, these compounds may desireably provide advantages for pharmaceutical uses, for example, with regard to one or more of their mechanisms of action, binding, inhibition efficacy, target selectivity, solubility, safety profiles, or bioavailability. Also needed are new formulations and methods of treatment which utilize these compounds.

### BRIEF DESCRIPTION OF THE INVENTION

Briefly, in one aspect, the present invention discloses compounds of Formula I,

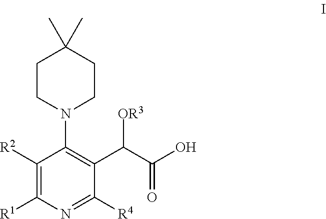

and pharmaceutically acceptable salts thereof, wherein:
R¹ is hydrogen, halo, cyano, C₁₋₁₀alkyl, C₁₋₁₀haloalkyl, —C₁₋₁₀alkyl-OH, HO—C₁₋₁₀alkyl-O—, Ar¹, —N(R⁵)(R⁶), —C(O)N(R⁷)(R⁸), or (R⁹)(R¹⁰)NC₁₋₁₀alkyl-;
provided R¹ and R⁴ are not both alkyl;
R² is benzodioxolyl, naphthalenyl, phenyl, pyrazinyl, pyridazinyl, pyridinyl, pyrimidinyl, quinolinyl, isoquinolinyl, tetrazinyl, or triazinyl, and is optionally substituted with 1-4 substituents independently selected from cyano, carbamoyl, carboxyl, halo, hydroxy, C₁₋₁₀alkyl, C₁₋₁₀haloalkyl, —N(R⁵)(R⁷), C₁₋₁₀alkyl-O—, Ar⁴, Ar⁴—C₁₋₁₀alkyl-O—, (R⁵)(Ar⁴—C₁₋₁₀alkyl)N—, Ar⁴—O—C₁₋₁₀alkyl-, or (Ar⁴)(R⁵)N—C₁₋₁₀alkyl-;
R³ is C₁₋₁₀alkyl;
R⁴ is hydrogen, cyano, halo, C₁₋₁₀haloalkyl, C₁₋₁₀alkyl, C₁₋₁₀alkyl-O—, C₁₋₁₀alkenyl, NH₂, hydroxy, —C₁₋₁₀alkyl-OH, carbamoyl, azetidinyl, pyrrolidinyl, piperidinyl, morpholinyl, or piperazinyl; provided R¹ and R⁴ are not both alkyl;
R⁵ is hydrogen, or C₁₋₁₀alkyl;
R⁶ is hydrogen, C₁₋₁₀alkyl, C₁₋₁₀alkyl-O—C₁₋₁₀alkyl-, C₁₋₁₀alkyl-O—C(O)—, C₃₋₉cycloalkyl, (C₃₋₉cycloalkyl)C₁₋₁₀alkyl-, 1-(C₁₋₁₀alkyl)piperidinyl-, tetrahydropyranyl, (tetrahydropyranyl)C₁₋₁₀alkyl-, morpholinoC₁₋₁₀alkyl-, (C₁₋₁₀alkyl)₂N—C₁₋₁₀alkyl-, piperidinylC₁₋₁₀alkyl-, 1-(C₁₋₁₀alkyl)piperidinylC₁₋₁₀alkyl-, 1-(C₁₋₁₀alkyl)piperazinylC₁₋₁₀alkyl-, Ar²—C₁₋₁₀alkyl-, Ar³, 1-(C₁₋₁₀alkylsulfonyl)piperidinyl-, or 1-(C₁₋₁₀alkylcarbonyl)piperidinyl-;
R⁷ is hydrogen, or C₁₋₁₀alkyl;
R⁸ is hydrogen, C₁₋₁₀alkyl, C₃₋₉cycloalkyl, (C₁₋₁₀alkyl)C₃₋₉cycloalkyl-, —SO₂(C₁₋₁₀alkyl), or —SO₂(C₃₋₉cycloalkyl);
R⁹ is hydrogen, or C₁₋₁₀alkyl;
R¹⁰ is hydrogen, C₁₋₁₀alkyl, (tetrahydropyranyl)C₁₋₁₀alkyl-, or C₁₋₁₀alkyl-O—C(O)—; or N(R⁷)(R⁸) taken together form an azetidinyl, pyrrolidinyl, piperidinyl, 1,1-dioxidothiomorpholinyl, or morpholinyl ring;
or N(R⁹)(R¹⁰) taken together form an azetidinyl, azocanyl, pyrrolidinyl, piperidinyl, or azaspirononanyl ring, and is optionally substituted with 1-3 C₁₋₁₀alkyl substitutents;
Ar¹ is imidazolyl, pyrazolyl, pyridinyl, pyrimidinyl, pyrrolyl, or dihydrocyclopentapyrazolyl and is optionally substituted with 1-3 substitutents independently selected from amino, C₁₋₁₀alkyl, or C₃₋₉cycloalkyl;
Ar² is imidazolyl, pyrazolyl, or pyridinyl, and is optionally substituted with 1-3 substitutents independently selected from C₁₋₁₀alkyl and halo substitutents;
Ar³ is phenyl, pyridinyl, pyrazolyl, pyridazinyl, or pyrimidinyl, and is optionally substituted with 1-3 substituents independently selected from C₁₋₁₀alkyl, halo, carboxy, and cyano; and
Ar⁴ is phenyl, benzofuropyrimidinyl, or pyridofuropyrimidinyl and is optionally substituted with 1-3 substituents independently selected from cyano, halo, C₁₋₁₀alkyl, and C₁₋₁₀alkyl-O—;
and wherein each reference to “haloalkyl includes all halogenated isomers from monohalo to perhalo.

The invention also provides a compound of Formula (I) or a pharmaceutically acceptable salt thereof for use in therapy.

The invention also provides a compound of Formula (I) or a pharmaceutically acceptable salt thereof for use in the treatment of HIV infection

The invention also provides the use of a compound of Formula (I) or a pharmaceutically acceptable salt thereof in the manufacture of a medicament for the treatment of HIV infection.

The invention also provides a pharmaceutical composition comprising a compound or salt of the invention.

In addition, the invention provides a method of treating HIV infection comprising administering a compound or salt of the invention to a patient.

In addition, the invention provides a method for inhibiting HIV integrase.

Also provided in accordance with the invention are methods for making the compounds and salts of the invention.

### DETAILED DESCRIPTION OF THE INVENTION

Preferably, R¹ is hydrogen, halo, cyano, C₁₋₁₀alkyl, C₁₋₁₀haloalkyl, —C₁₋₁₀alkyl-OH, Ar¹, —N(R⁵)(R⁶), or (R⁹)(R¹⁰)NC₁₋₁₀alkyl-; provided R¹ and R⁴ are not both alkyl; and wherein R⁵, R⁶, R⁹, and R¹⁰ are as defined above. More preferably, R¹ is hydrogen or (R⁹)(R¹⁰)NC₁₋₁₀alkyl-; provided R¹ and R⁴ are not both alkyl; and wherein R⁹ and R¹⁰ are as defined above. Most preferably R¹ is hydrogen.

Preferably, R² is phenyl, pyrazinyl, pyridazinyl, pyridinyl, pyrimidinyl, quinolinyl, or isoquinolinyl, and is optionally substituted with 1-4 substituents independently selected from cyano, carbamoyl, carboxyl, halo, hydroxy, C₁₋₁₀alkyl, C₁₋₁₀haloalkyl, —N(R⁵)(R⁷), C₁₋₁₀alkyl-O—, Ar⁴, Ar⁴—C₁₋₁₀alkyl-O—, (R⁵)(Ar⁴—C₁₋₁₀alkyl)N—, Ar⁴—O—C₁₋₁₀alkyl-, or (Ar⁴)(R⁵)N—C₁₋₁₀alkyl-; and wherein R⁵ and Ar⁴ are defined as above. More Preferably, R² is phenyl, pyrazinyl, pyridazinyl, pyridinyl, or pyrimidinyl, and is optionally substituted with 1-4 substituents independently selected from cyano, carbamoyl, carboxyl, halo, C₁₋₁₀alkyl, C₁₋₁₀haloalkyl, —N(C₁₋₁₀alkyl)₂, C₁₋₁₀alkyl-O—, Ar⁴, Ar⁴—C₁₋₁₀alkyl-O—, (R⁵)(Ar⁴—C₁₋₁₀alkyl)N—, Ar⁴—O—C₁₋₁₀alkyl-, or (Ar⁴)(R⁵)N—C₁₋₁₀alkyl-; and wherein R⁵ and Ar⁴ are defined as above. Most preferably, R² is pyridinyl substituted with one C₁₋₁₀alkyl-O—. Preferably, the C₁₋₁₀alkyl-O— is a C₄alkyl.

Preferably, the R³ is a C₄alkyl.

Preferably, R⁴ is hydrogen, cyano, halo, C₁₋₁₀haloalkyl, C₁₋₁₀alkyl, C₁₋₁₀alkyl-O—, C₁₋₁₀alkenyl, hydroxy, or —C₁₋₁₀alkyl-OH; provided R¹ and R⁴ are not both alkyl. More preferably, R⁴ is C₁₋₁₀alkyl, cyano, halo, or C₁₋₁₀haloalkyl; provided R¹ and R⁴ are not both alkyl. Most preferably R⁴ is methyl and R¹ is hydrogen.

In one embodiment, there is provided compounds of Formula IA,

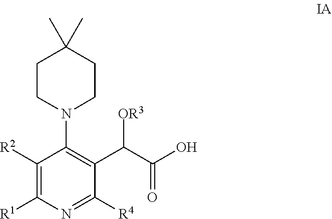

and pharmaceutically acceptable salts thereof, wherein:
R¹ is hydrogen, halo, cyano, C₁₋₆alkyl, C₁₋₆haloalkyl, —C₁₋₆alkyl-OH, HO—C₁₋₆alkyl-O—, Ar¹, —N(R⁵)(R⁶), —C(O)N(R⁷)(R⁸), or (R⁹)(R¹⁰)NC₁₋₆alkyl-;
provided R¹ and R⁴ are not both alkyl;
R² is benzodioxolyl, naphthalenyl, phenyl, pyrazinyl, pyridazinyl, pyridinyl, pyrimidinyl, quinolinyl, isoquinolinyl, tetrazinyl, or triazinyl, and is optionally substituted with 1-4 substituents independently selected from cyano, carbamoyl, carboxyl, halo, C₁₋₆alkyl, C₁₋₆haloalkyl, —N(C₁₋₆alkyl)₂, C₁₋₆alkyl-O—, Ar⁴, Ar⁴—C₁₋₆alkyl-O—, (R⁵)(Ar⁴—C₁₋₆alkyl)N—, Ar⁴—O—C₁₋₆alkyl-, or (Ar⁴)(R⁵)N—C₁₋₆alkyl-;
R³ is C₁₋₆alkyl;
R⁴ is hydrogen, cyano, halo, C₁₋₆haloalkyl, C₁₋₆alkyl, C₁₋₆alkyl-O—, C₁₋₆alkenyl, NH₂, hydroxy, —C₁₋₆alkyl-OH, carbamoyl, azetidinyl, pyrrolidinyl, piperidinyl, morpholinyl, or piperazinyl; provided R¹ and R⁴ are not both alkyl;
R⁵ is hydrogen, or C₁₋₆alkyl;
R⁶ is hydrogen, C₁₋₆alkyl, C₁₋₆alkyl-O—C₁₋₆alkyl-, C₁₋₆alkyl-O—C(O)—, C₃₋₆cycloalkyl, (C₃₋₆cycloalkyl)C₁₋₆alkyl-, 1-(C₁₋₆alkyl)piperidinyl-, tetrahydropyranyl, (tetrahydropyranyl)C₁₋₆alkyl-, morpholinoC₁₋₆alkyl-, (C₁₋₆alkyl)₂N—C₁₋₆alkyl-, piperidinylC₁₋₆alkyl-, 1-(C₁₋₆alkyl)piperidinylC₁₋₆alkyl-, 1-(C₁₋₆alkyl)piperazinylC₁₋₆alkyl-, Ar²—C₁₋₆alkyl-, Ar³, 1-(C₁₋₆alkylsulfonyl)piperidinyl-, or

### 1-(C₁₋₆alkylcarbonyl)piperidinyl-;

R⁷ is hydrogen, or C₁₋₆alkyl;
R⁸ is hydrogen, C₁₋₆alkyl, C₃₋₆cycloalkyl, (C₁₋₆alkyl)C₃₋₆cycloalkyl-, —SO₂(C₁₋₆alkyl), or —SO₂(C₃₋₆cycloalkyl);
R⁹ is hydrogen, or C₁₋₆alkyl;
R¹⁰ is hydrogen, C₁₋₆alkyl, (tetrahydropyranyl)C₁₋₆alkyl-, or C₁₋₆alkyl-O—C(O)—;
(R⁹)(R¹⁰)N taken together form an azetidinyl, pyrrolidinyl, piperidinyl, 1,1-dioxidothiomorpholinyl, or morpholinyl ring;
(R⁹)(R¹⁰)N taken together form an azetidinyl, azocanyl, pyrrolidinyl, piperidinyl, or azaspirononanyl ring, and is optionally substituted with 1-3 C₁₋₆alkyl substitutents;
Ar¹ is imidazolyl, pyrazolyl, pyridinyl, pyrimidinyl, pyrrolyl, or dihydrocyclopentapyrazolyl and is optionally substituted with 1-3 substitutents independently selected from amino, C₁₋₆alkyl, or C₃₋₆cycloalkyl;
Ar² is imidazolyl, pyrazolyl, or pyridinyl, and is optionally substituted with 1-3 substitutents independently selected from C₁₋₆alkyl and halo substitutents;
Ar³ is phenyl, pyridinyl, pyrazolyl, pyridazinyl, or pyrimidinyl, and is optionally substituted with 1-3 substituents independently selected from C₁₋₆alkyl, halo, carboxy, and cyano; and
Ar⁴ is phenyl, benzofuropyrimidinyl, or pyridofuropyrimidinyl and is optionally substituted with 1-3 substituents independently selected from cyano, halo, C₁₋₆alkyl, and C₁₋₆alkyl-O—;
and wherein each reference to “haloalkyl includes all halogenated isomers from monohalo to perhalo.

Preferably, R¹ is hydrogen, halo, cyano, C₁₋₆alkyl, C₁₋₆haloalkyl, —C₁₋₆alkyl-OH, Ar¹, —N(R⁵)(R⁶), or (R⁹)(R¹⁰)NC₁₋₆alkyl-; provided R¹ and R⁴ are not both alkyl; and wherein R⁵, R⁶, R⁹, and R¹⁰ are as defined above for the compounds of formula (IA). More preferably, R¹ is hydrogen or (R⁹)(R¹⁰)NC₁₋₆alkyl-; provided R¹ and R⁴ are not both alkyl; and wherein R⁹ and R¹⁰ are as defined above for the compounds of formula (IA).

Preferably, R² is phenyl, pyrazinyl, pyridazinyl, pyridinyl, pyrimidinyl, quinolinyl, or isoquinolinyl, and is optionally substituted with 1-4 substituents independently selected from cyano, carbamoyl, carboxyl, halo, C₁₋₆alkyl, C₁₋₆haloalkyl, —N(C₁₋₆alkyl)₂, C₁₋₆alkyl-O—, Ar⁴, Ar⁴—C₁₋₆alkyl-O—, (R⁵)(Ar⁴—C₁₋₆alkyl)N—, Ar⁴—O—C₁₋₆alkyl-, or (Ar⁴)(R⁵)N—C₁₋₆alkyl-; and wherein R⁵ and Ar⁴ are defined as above for the compounds of formula (IA). More Preferably, R² is phenyl, pyrazinyl, pyridazinyl, pyridinyl, or pyrimidinyl, and is optionally substituted with 1-4 substituents independently selected from cyano, carbamoyl, carboxyl, halo, C₁₋₆alkyl, C₁₋₆haloalkyl, —N(C₁₋₆alkyl)₂, C₁₋₆alkyl-O—, Ar⁴, Ar⁴—C₁₋₆alkyl-O—, (R⁵)(Ar⁴—C₁₋₆alkyl)N—, Ar⁴—O—C₁₋₆alkyl-, or (Ar⁴)(R⁵)N—C₁₋₆alkyl-; and wherein R⁵ and Ar⁴ are defined as above for the compounds of formula (IA).

Preferably, R⁴ is hydrogen, cyano, halo, C₁₋₆haloalkyl, C₁₋₆alkyl, C₁₋₆alkyl-O—, C₁₋₆alkenyl, hydroxy, or —C₁₋₆alkyl-OH; provided R¹ and R⁴ are not both alkyl. More preferably, R⁴ is C₁₋₆alkyl, cyano, halo, or C₁₋₆haloalkyl; provided R¹ and R⁴ are not both alkyl.

In one embodiment there is provided (2S)-2-(tert-butoxy)-2-[4′-(4,4-dimethylpiperidin-1-yl)-6′-methyl-5-(2-methylpropoxy)-[2,3′-bipyridine]-5′-yl]acetic acid of formula

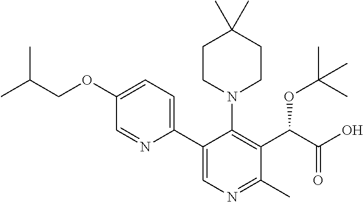 [SMILES: Cc1ncc(-c2ccc(OCC(C)C)cn2)c(N2CCC(C)(C)CC2)c1[C@H](OC(C)(C)C)C(=O)O]

or a pharmaceutically acceptable salt thereof.

In one embodiment there is provided (2S)-2-(tert-butoxy)-2-[4′-(4,4-dimethylpiperidin-1-yl)-6′-methyl-5-(2-methylpropoxy)-[2,3′-bipyridine]-5′-yl]acetic acid.

In one embodiment there is provided (2S)-2-(tert-butoxy)-2-[5-butoxy-4′-(4,4-dimethylpiperidin-1-yl)-6′-methyl-[2,3′-bipyridine]-5′-yl]acetic acid of formula

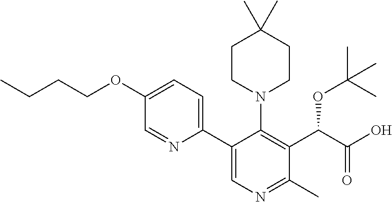 [SMILES: CCCCOc1ccc(-c2cnc(C)c([C@H](OC(C)(C)C)C(=O)O)c2N2CCC(C)(C)CC2)nc1]

or a pharmaceutically acceptable salt thereof.

In one embodiment there is provided (2S)-2-(tert-butoxy)-2-[5-butoxy-4′-(4,4-dimethylpiperidin-1-yl)-6′-methyl-[2,3′-bipyridine]-5′-yl]acetic acid

In one embodiment there is a provided a compound or pharmaceutically acceptable salt thereof selected from the group consisting of Examples 1-442 or pharmaceutically acceptable salts thereof.

The invention includes all pharmaceutically acceptable salt forms of the compounds. Pharmaceutically acceptable salts are those in which the counter ions do not contribute significantly to the physiological activity or toxicity of the compounds and as such function as pharmacological equivalents. These salts can be made according to common organic techniques employing commercially available reagents. Some anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate. Some cationic salt forms include ammonium, aluminum, benzathine, bismuth, calcium, choline, diethylamine, diethanolamine, lithium, magnesium, meglumine, 4-phenylcyclohexylamine, piperazine, potassium, sodium, tromethamine, and zinc.

Some of the compounds of the invention exist in stereoisomeric forms. The invention includes all stereoisomeric forms of the compounds including enantiomers and diastereromers. Methods of making and separating stereoisomers are known in the art. The invention includes all tautomeric forms of the compounds. The invention includes atropisomers and rotational isomers.

The invention is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. Such compounds may have a variety of potential uses, for example as standards and reagents in determining biological activity. In the case of stable isotopes, such compounds may have the potential to favorably modify biological, pharmacological, or pharmacokinetic properties.

In one embodiment, a method for treating or preventing an HIV infection in a patient having or at risk of having the infection is provided, comprising administering to the patient a therapeutically effective amount of a compound disclosed herein, or a pharmaceutically acceptable salt thereof, in combination with a therapeutically effective amount of one or more additional therapeutic agents.

In one embodiment, a method for treating or preventing an HIV infection in a human having or at risk of having the infection is provided, comprising administering to the human a therapeutically effective amount of (2S)-2-(tert-butoxy)-2-[4′-(4,4-dimethylpiperidin-1-yl)-6′-methyl-5-(2-methylpropoxy)-[2,3′-bipyridine]-5′-yl]acetic acid or a pharmaceutically acceptable salt thereof, in combination with a therapeutically effective amount of one or more additional therapeutic agents.

In one embodiment, a method for treating or preventing an HIV infection in a human having or at risk of having the infection is provided, comprising administering to the human a therapeutically effective amount of (2S)-2-(tert-butoxy)-2-[5-butoxy-4′-(4,4-dimethylpiperidin-1-yl)-6′-methyl-[2,3′-bipyridine]-5′-yl]acetic acid or a pharmaceutically acceptable salt thereof, in combination with a therapeutically effective amount of one or more additional therapeutic agents.

In one embodiment there is provided of (2S)-2-(tert-butoxy)-2-[4′-(4,4-dimethylpiperidin-1-yl)-6′-methyl-5-(2-methylpropoxy)-[2,3′-bipyridine]-5′-yl]acetic acid or a pharmaceutically acceptable salt thereof for use in the treatment of HIV infection.

In one embodiment there is provided (2S)-2-(tert-butoxy)-2-[5-butoxy-4′-(4,4-dimethylpiperidin-1-yl)-6′-methyl-[2,3′-bipyridine]-5′-yl]acetic acid or a pharmaceutically acceptable salt thereof for use in the treatment of HIV infection.

In one embodiment there is provided the use of (2S)-2-(tert-butoxy)-2-[4′-(4,4-dimethylpiperidin-1-yl)-6′-methyl-5-(2-methylpropoxy)-[2,3′-bipyridine]-5′-yl]acetic acid or a pharmaceutically acceptable salt thereof in the manufacture of a medicament for the treatment of HIV infection.

In one embodiment there is provided the use of (2S)-2-(tert-butoxy)-2-[5-butoxy-4′-(4,4-dimethylpiperidin-1-yl)-6′-methyl-[2,3′-bipyridine]-5′-yl]acetic acid or a pharmaceutically acceptable salt thereof in the manufacture of a medicament for the treatment of HIV infection.

In one embodiment, pharmaceutical compositions comprising a compound disclosed herein, or a pharmaceutically acceptable salt thereof, in combination with one or more additional therapeutic agents, and a pharmaceutically acceptable carrier, diluent or excipient are provided.

In one embodiment, pharmaceutical compositions comprising a compound disclosed herein, or a pharmaceutically acceptable salt thereof, in combination with one or more additional therapeutic agents, and a pharmaceutically acceptable carrier, diluent or excipient are provided.

In one embodiment, combination pharmaceutical agents comprising a compound disclosed herein, or a pharmaceutically acceptable salt thereof, in combination with one or more additional therapeutic agents are provided.

In the above embodiments, the additional therapeutic agent may be an anti-HIV agent. For example, in some embodiments, the additional therapeutic agent is selected from the group consisting of HIV protease inhibitors, HIV non-nucleoside inhibitors of reverse transcriptase, HIV nucleoside inhibitors of reverse transcriptase, HIV nucleotide inhibitors of reverse transcriptase, HIV integrase inhibitors, HIV non-catalytic site (or allosteric) integrase inhibitors, entry inhibitors (e.g., CCR5 inhibitors, gp41 inhibitors (i.e., fusion inhibitors) and CD4 attachment inhibitors), CXCR4 inhibitors, gp120 inhibitors, G6PD and NADH-oxidase inhibitors, compounds that target the HIV capsid (“capsid inhibitors”; e.g., capsid polymerization inhibitors or capsid disrupting compounds such as those disclosed in WO 2013/006738 (Gilead Sciences), US 2013/0165489 (University of Pennsylvania), and WO 2013/006792 (Pharma Resources), pharmacokinetic enhancers, and other drugs for treating HIV, and combinations thereof.

In further embodiments, the additional therapeutic agent is selected from one or more of:

(1) HIV protease inhibitors selected from the group consisting of amprenavir, atazanavir, fosamprenavir, indinavir, lopinavir, ritonavir, nelfinavir, saquinavir, tipranavir, brecanavir, darunavir, TMC-126, TMC-114, mozenavir (DMP-450), JE-2147 (AG1776), L-756423, RO0334649, KNI-272, DPC-681, DPC-684, GW640385X, DG17, PPL-100, DG35, and AG 1859;
(2) HIV non-nucleoside or non-nucleotide inhibitors of reverse transcriptase selected from the group consisting of capravirine, emivirine, delaviridine, efavirenz, nevirapine, (+) calanolide A, etravirine, GW5634, DPC-083, DPC-961, DPC-963, MIV-150, TMC-120, rilpivirene, BILR 355 BS, VRX 840773, lersivirine (UK-453061), RDEA806, KM023 and MK-1439;
(3) HIV nucleoside inhibitors of reverse transcriptase selected from the group consisting of zidovudine, emtricitabine, didanosine, stavudine, zalcitabine, lamivudine, abacavir, amdoxovir, elvucitabine, alovudine, MIV-210, .+−.-FTC, D-d4FC, emtricitabine, phosphazide, fozivudine tidoxil, apricitibine (AVX754), KP-1461, GS-9131 (Gilead Sciences) and fosalvudine tidoxil (formerly HDP 99.0003);
(4) HIV nucleotide inhibitors of reverse transcriptase selected from the group consisting of tenofovir, tenofovir disoproxil fumarate, tenofovir alafenamide fumarate (Gilead Sciences), GS-7340 (Gilead Sciences), GS-9148 (Gilead Sciences), adefovir, adefovir dipivoxil, CMX-001 (Chimerix) or CMX-157 (Chimerix);
(5) HIV integrase inhibitors selected from the group consisting of curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, S-1360, AR-177, L-870812, and L-870810, raltegravir, BMS-538158, GSK364735C, BMS-707035, NMK-2048, BA 011, elvitegravir, dolutegravir and GSK-744;
(6) HIV non-catalytic site, or allosteric, integrase inhibitors (NCINI) including, but not limited to, BI-224436, CX0516, CX05045, CX14442, compounds disclosed in WO 2009/062285 (Boehringer Ingelheim), WO 2010/130034 (Boehringer Ingelheim), WO 2013/159064 (Gilead Sciences), WO 2012/145728 (Gilead Sciences), WO 2012/003497 (Gilead Sciences), WO 2012/003498 (Gilead Sciences) each of which is incorporated by references in its entirety herein;
(7) gp41 inhibitors selected from the group consisting of enfuvirtide, sifuvirtide, albuvirtide, FB006M, and TRI-1144;
(8) the CXCR4 inhibitor AMD-070;
(9) the entry inhibitor SP01A;
(10) the gp120 inhibitor BMS-488043;
(11) the G6PD and NADH-oxidase inhibitor immunitin;
(12) CCR5 inhibitors selected from the group consisting of aplaviroc, vicriviroc, maraviroc, cenicriviroc, PRO-140, INCB 115050, PF-232798 (Pfizer), and CCR5 mAb004;
(13) CD4 attachment inhibitors selected from the group consisting of ibalizumab (TMB-355) and BMS-068 (BMS-663068);
(14) pharmacokinetic enhancers selected from the group consisting of cobicistat and SPI-452; and
(15) other drugs for treating HIV selected from the group consisting of BAS-100, SPI-452, REP 9, SP-01A, TNX-355, DES6, ODN-93, ODN-112, VGV-1, PA-457 (bevirimat), HRG214, VGX-410, KD-247, AMZ 0026, CYT 99007A-221 HIV, DEBIO-025, BAY 50-4798, MDX010 (ipilimumab), PBS 119, ALG 889, and PA-1050040 (PA-040),
and combinations thereof.

Unless specified otherwise, these terms have the following meanings.

“Combination,” “coadministration,” “concurrent” and similar terms referring to the administration of a compound of Formula I with at least one anti-HIV agent mean that the components are part of a combination antiretroviral therapy or highly active antiretroviral therapy (“HAART”) as understood by practitioners in the field of AIDS and HIV infection.

“Therapeutically effective” means the amount of agent required to provide a benefit to a patient as understood by practitioners in the field of AIDS and HIV infection. In general, the goals of treatment are suppression of viral load, restoration and preservation of immunologic function, improved quality of life, and reduction of HIV-related morbidity and mortality.

“Patient” means a person infected with the HIV virus.

“Treatment,” “therapy,” “regimen,” “HIV infection,” “ARC,” “AIDS” and related terms are used as understood by practitioners in the field of AIDS and HIV infection.

Those terms not specifically set forth herein shall have the meaning which is commonly understood and accepted in the art.

Solid compositions which are normally formulated in dosage units and compositions providing from about 1 to 1000 milligram (“mg”) of the active ingredient per dose are typical. Some examples of dosages are 1 mg, 10 mg, 100 mg, 250 mg, 500 mg, and 1000 mg. Generally, other antiretroviral agents will be present in a unit range similar to agents of that class used clinically. Typically, this is about 0.25-1000 mg/unit.

Liquid compositions are usually in dosage unit ranges. Generally, the liquid composition will be in a unit dosage range of about 1-100 milligram per milliliter (“mg/mL”). Some examples of dosages are 1 mg/mL, 10 mg/mL, 25 mg/mL, 50 mg/mL, and 100 mg/mL. Generally, other antiretroviral agents will be present in a unit range similar to agents of that class used clinically. Typically, this is about 1-100 mg/mL.

The invention encompasses all conventional modes of administration; oral and parenteral methods are preferred. Generally, the dosing regimen will be similar to other antiretroviral agents used clinically. Typically, the daily dose will be about 1-100 milligram per kilogram (“mg/kg”) body weight daily. Generally, more compound is required orally and less parenterally. The specific dosing regimen, however, will be determined by a physician using sound medical judgment.

### Methods of Synthesis

The compounds of this invention can be made by various methods known in the art including those of the following schemes and in the specific embodiments section. The structure numbering and variable numbering shown in the synthetic schemes are distinct from, and should not be confused with, the structure or variable numbering in the claims or the rest of the specification. The variables in the schemes are meant only to illustrate how to make some of the compounds of this invention. The disclosure is not limited to the foregoing illustrative examples and the examples should be considered in all respects as illustrative and not restrictive, reference being made to the appended claims, rather than to the foregoing examples, and all changes which come within the meaning and range of equivalency of the claims are therefore intended to be embraced.

Abbreviations used in the schemes and examples generally follow conventions used in the art. Chemical abbreviations used in the specification and examples are defined as follows: “KHMDS” for potasium bis(trimethylsilyl)amide; “DMF” for N,N-dimethylformamide; “HATU” for O-(t-Azabenzotriazol-1-yl)-N,N,N′,N′-tetramethyluronium hexafluorophosphate, “MeOH” for methanol; “Ar” for aryl; “TFA” for trifluoroacetic acid, “DMSO” for dimethylsulfoxide; “h” for hours; “rt” for room temperature or retention time (context will dictate); “min” for minutes; “EtOAc” for ethyl acetate; “THF” for tetrahydrofuran; “Et₂O” for diethyl ether; “DMAP” for 4-dimethylaminopyridine; “DCE” for 1,2-dichloroethane; “ACN” for acetonitrile; “DME” for 1,2-dimethoxyethane; “HOBt” for 1-hydroxybenzotriazole hydrate; and “DIEA” for diisopropylethylamine.

Certain other abbreviations as used herein, are defined as follows: “1×” for once, “2×” for twice, “3×” for thrice, “° C.” for degrees Celsius, “eq” for equivalent or equivalents, “g” for gram or grams, “mg” for milligram or milligrams, “L” for liter or liters, “mL” for milliliter or milliliters, “μL” for microliter or microliters, “N” for normal, “M” for molar, “mmol” for millimole or millimoles, “atm” for atmosphere, “psi” for pounds per square inch, “conc.” for concentrate, “sat” or “sat'd” for saturated, “MW” for molecular weight, “mp” for melting point, “ee” for enantiomeric excess, “MS” or “Mass Spec” for mass spectrometry, “ESI” for electrospray ionization mass spectroscopy, “HR” for high resolution, “HRMS” for high resolution mass spectrometry, “LCMS” for liquid chromatography mass spectrometry, “HPLC” for high pressure liquid chromatography, “RP HPLC” for reverse phase HPLC, “TLC” or “tlc” for thin layer chromatography, “NMR” for nuclear magnetic resonance spectroscopy, “¹H” for proton, “δ” for delta, “s” for singlet, “d” for doublet, “t” for triplet, “q” for quartet, “m” for multiplet, “br” for broad, “Hz” for hertz, and “α”, “β”, “R”, “S”, “E”, and “Z” are stereochemical designations familiar to one skilled in the art.

Some compounds can be synthesized from an appropriately substituted heterocycle I-1 according to Scheme I. Compounds I-1 and I-6 are commercially available or synthesized by reactions well known in the art. Treatment of compound I-1 with bromine provided the dibromo intermediates I-2 which was converted to the chloropyridine I-3 by reacting with POCl₃. Intermediate I-3 conveniently transformed to ketoester I-5 using conditions well-known to those skilled in the art, including reacting I-3 with Grignard reagent in the presence of catalytic copper(I) bromide dimethylsulfide complex followed by alkyl 2-chloro-2-oxoacetate. Coupling of amines 1-5 with intermediate 1-6 in the presence of an organic base such as Hunig's base provided intermediate I-7. Chiral Lewis acid such as I-8 mediated reduction of ketoester I-7 with catecholborane furnished the chiral alcohol I-9. Tertiary butylation of alcohol I-9 by well-known conditions, including but not limited to tertiary-butyl acetate and perchloric acid, gave intermediate I-10. Intermediates I-10 are conveniently transformed to intermediates I-11 using conditions well-known in the art, including but not limited to the Suzuki coupling between intermediates I-10 and R⁶B(OR)₂. The boronate or boronic acid coupling reagents, well-known in the art, are commercially available or are prepared by reactions well-known to those skilled in the art. Hydrolysis of intermediate I-11 by using conditions well-known to those skilled in the art furnished the carboxylic acid I-12.

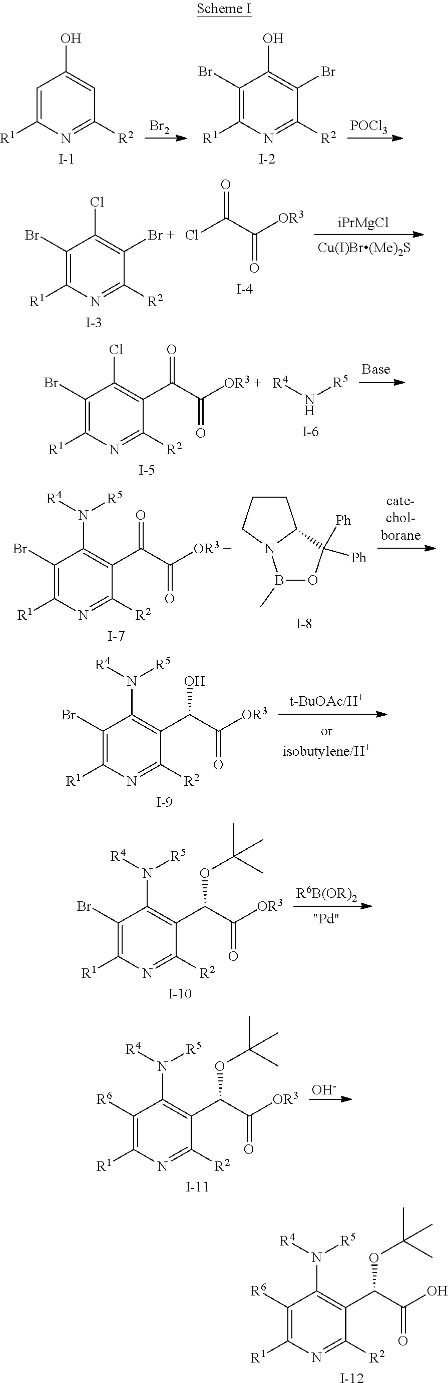

Intermediates I-10 are conveniently transformed to intermediates II-2 using conditions well-known in the art, including but not limited to the Suzuki coupling between intermediates I-10 and II-1. Cleavage of protecting group in II-2 provided phenol II-3. Alkylation of the phenol II-3 was achieved by using conditions well known to those skilled in the art, including but not limited to Mitshunobu reaction to provide the intermediate II-4. Hydrolysis of intermediate II-4 by using conditions well-known in the literature furnished carboxylic acid II-5.

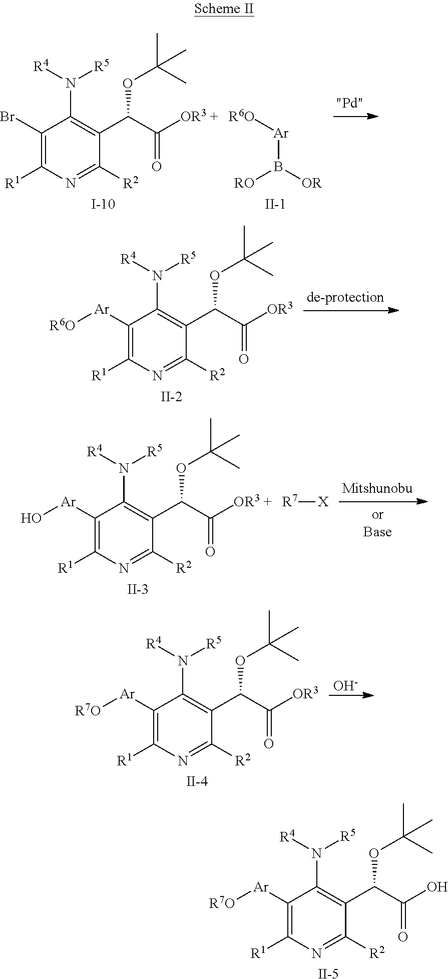

In yet another method, some compounds of this invention can be synthesized according to Scheme III. Pyridine III-1, can be produced using methods similar to those described in the previous schemes. This intermediate can be carried on to the final products by a variety of paths. In one, the C2 and C6 alkyl groups can be oxidized to furnish intermediates III-3 and/or III-4 which can be further transformed to final compounds III-9 or III-10 by methods well known in the art.

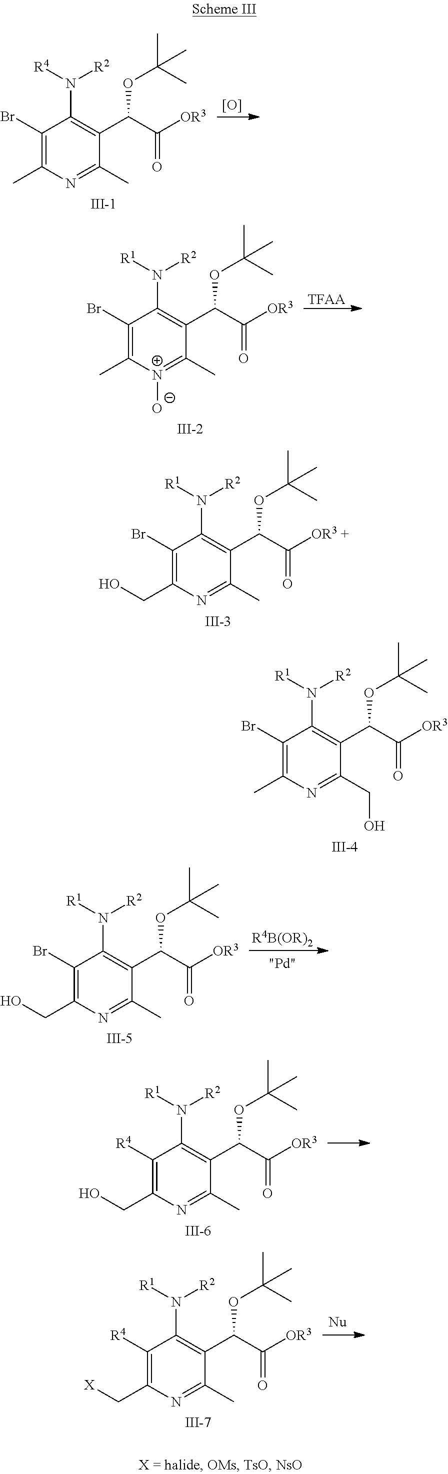

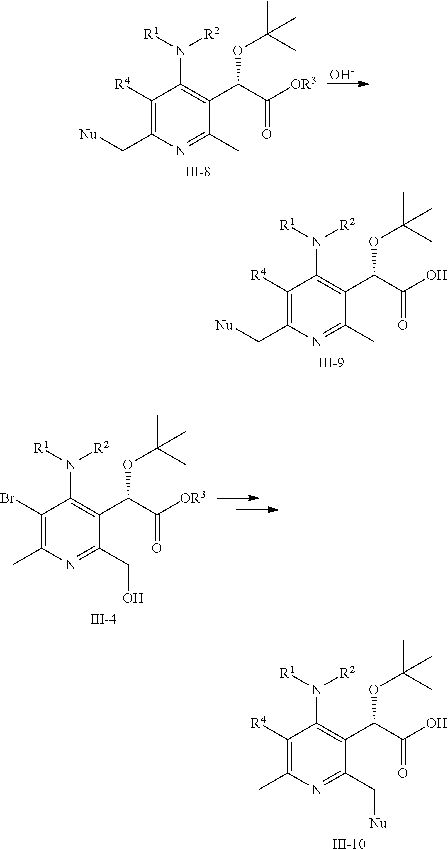

In yet another process, some compounds of this invention can be synthesized according to Scheme IV. Pyridine III-5 can be transformed to the final products by several paths. In one path, the C6 hydroxymethyl is oxidized to furnish carboxylic acid IV-1 which upon heating in the presence of acid provided C6-desmethyl analog IV-2. The “Pd” mediated coupling of boronate IV-2 with appropriate aryl halides or aryl triflate followed by hydrolysis furnished the target compounds. Alternatively, the target compounds could be synthesized by coupling intermediate IV-2 with aryl halides under Negishi coupling conditions followed by ester hydrolysis.

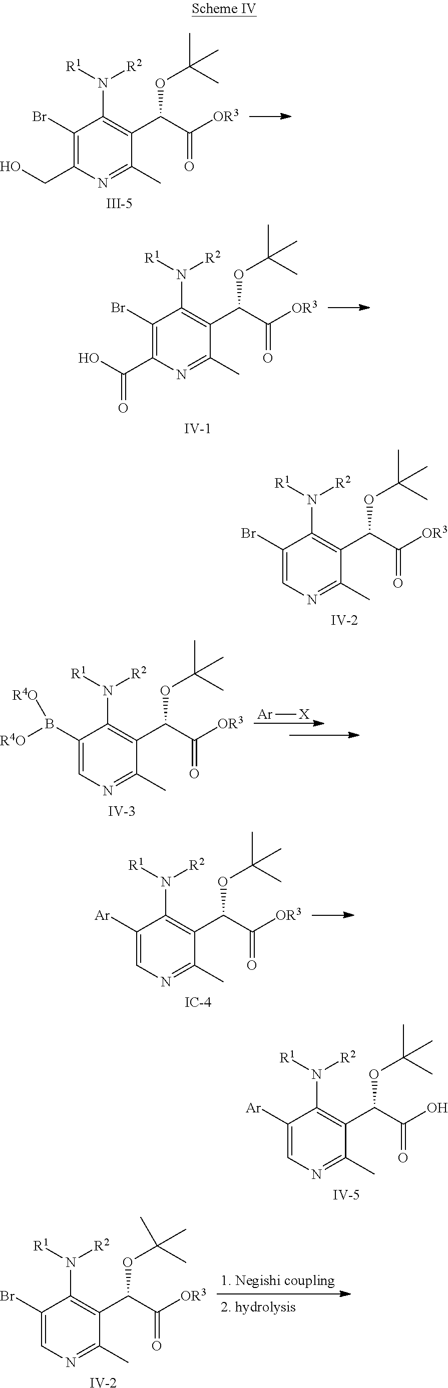

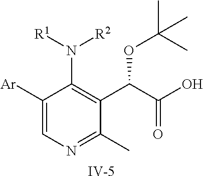

The compounds described herein were purified by the methods well known to those skilled in art by normal phase column chromatography on silica gel column using appropriate solvent system described. Preparative HPLC or preparative LC/MS purifications mentioned in this experimentation section were carried out gradient elution either on Sunfire Prep C18 ODB column (5 μm; 19 or 30×100 mm) or Waters Xbridge C18 column (5 μM; 19×200 or 30×100 mm) or Water Atlantis (5 μm; 19 or 30×100 mm) or Waters XBridge C18, 2.1 mm×50 mm, 1.7 μm particles using the following mobile phases. Mobile phase A: 9:1 H₂O/acetonitrile with 10 mM NH₄OAc and mobile phase B:A:9:1 acetonitrile/H₂O with 10 mM NH₄OAc; or mobile phase A: 9:1 H₂O/acetonitrile with 0.1% TFA and mobile phase B:A:9:1 acetonitrile/H₂O with 0.1% TFA; or mobile phase A: water/MeOH (9:1) with 20 mM NH₄OAc and mobile phase B: 95:5 MeOH/H₂O with 20 mM NH₄OAc or mobile phase A: water/MeOH (9:1) with 0.1% TFA and mobile phase B:95:5 MeOH/H₂O with 0.1% TFA or mobile Phase A: 5:95 acetonitrile:water with 10-mM ammonium acetate; Mobile Phase B: 95:5 acetonitrile:water with 10-mM ammonium acetate.

Compounds purified by preparative HPLC were diluted in methanol (1.2 mL) or DMF and purified using a Shimadzu LC-8A or LC-10A automated preparative HPLC system.

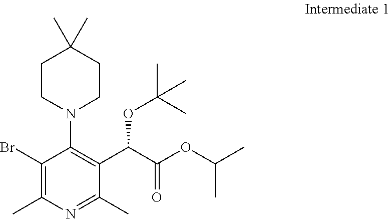 [SMILES: Cc1nc(C)c([C@H](OC(C)(C)C)C(=O)OC(C)C)c(N2CCC(C)(C)CC2)c1Br]

(S)-Isopropyl 2-(5-bromo-4-(4,4-dimethylpiperidin-1-yl)-2,6-dimethylpyridin-3-yl)-2-(tert-butoxy)acetate: was prepared according to the procedure described in WO2015126726.

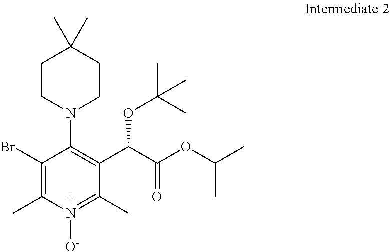

### (S)-3-Bromo-5-(1-(tert-butoxy)-2-isopropoxy-2-oxoethyl)-4-(4,4-dimethylpiperidin-1-yl)-2,6-dimethylpyridine 1-oxide

To a stirred solution of (S)-isopropyl 2-(5-bromo-4-(4,4-dimethylpiperidin-1-yl)-2,6-dimethylpyridin-3-yl)-2-(tert-butoxy)acetate (15 g, 32.0 mmol) in DCM (150 mL) was added 77% mCPBA (10.74 g, 47.9 mmol) at rt over 5 min. After 4 h, the reaction mixture was washed with 1M NaOH (2×100 mL), dried (MgSO₄), filtered and concentrated to give (S)-3-bromo-5-(1-(tert-butoxy)-2-isopropoxy-2-oxoethyl)-4-(4,4-dimethylpiperidin-1-yl)-2,6-dimethylpyridine 1-oxide (15.3 g, 31.5 mmol, 99% yield) which was used in the next step without purification. LCMS (M+1)=485.1 and 487.1.

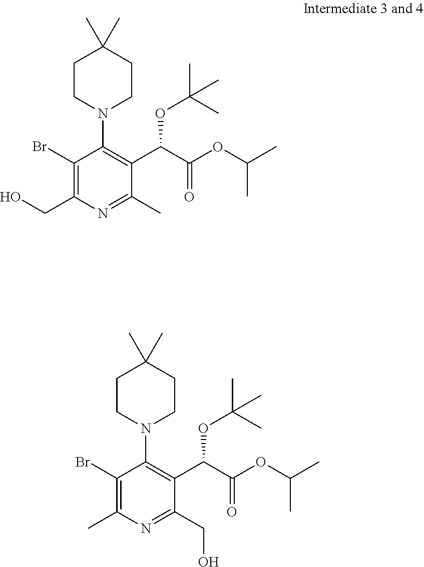 [SMILES: Cc1nc(CO)c(Br)c(N2CCC(C)(C)CC2)c1[C@H](OC(C)(C)C)C(=O)OC(C)C ; Cc1nc(CO)c([C@H](OC(C)(C)C)C(=O)OC(C)C)c(N2CCC(C)(C)CC2)c1Br]

### (S)-Isopropyl 2-(5-bromo-4-(4,4-dimethylpiperidin-1-yl)-6-(hydroxymethyl)-2-methylpyridin-3-yl)-2-(tert-butoxy)acetate (intermediate 3) and (S)-isopropyl 2-(5-bromo-4-(4,4-dimethylpiperidin-1-yl)-2-(hydroxymethyl)-6-methylpyridin-3-yl)-2-(tert-butoxy)acetate (Intermediate 4)

To a stirred solution of (S)-3-bromo-5-(1-(tert-butoxy)-2-isopropoxy-2-oxoethyl)-4-(4,4-dimethylpiperidin-1-yl)-2,6-dimethylpyridine 1-oxide (5.24 g, 10.79 mmol) anhydrous DCM (50 ml) was added trifluoroacetic anhydride (3.05 ml, 21.59 mmol) at RT. After 3 h, sat NaHCO₃ (50 mL) was added and stirred vigorously for 10 minutes. The solution phases were separated and organic phase collected and volatiles evaporated. The residue was taken up in EtOAc and washed with 50 mL of 1 M HCl followed by a wash with sat. sodium bicarbonate. The organic layer was then washed with brine, dried over MgSO₄, filtered and volatiles evaporated to afford the crude as an orange oil. The crude product was purified via silica gel (120 g column, 5-20% EtOAc:Hex) to give two products:

### (S)-Isopropyl 2-(5-bromo-4-(4,4-dimethylpiperidin-1-yl)-6-(hydroxymethyl)-2-methylpyridin-3-yl)-2-(tert-butoxy)acetate (intermediate 3)

Clear oil that later crystallized, 4.0 g (76%). ¹H NMR (500 MHz, CDCl₃) δ 6.25 (br. s., 1H), 5.06 (spt, J=6.3 Hz, 1H), 4.75-4.79 (m, 1H), 4.74-4.62 (m, 2H), 4.02-4.12 (br. s., 1H), 3.54-3.46 (m, 1H), 2.93 (d, J=11.5 Hz, 1H), 2.70-2.63 (m, 1H), 2.61 (s, 3H), 1.65-1.56 (m, 2H), 1.50-1.43 (m, 1H), 1.35-1.40 (m, 1H), 1.23 (d, J=6.2 Hz, 3H), (1.22 (s, 9H), 1.16 (d, J=6.3 Hz, 3H), 1.09 (s, 3H), 1.05 (s, 3H). LCMS (M+H)=485.35 and 487.2.

### (S)-Isopropyl 2-(5-bromo-4-(4,4-dimethylpiperidin-1-yl)-2-(hydroxymethyl)-6-methylpyridin-3-yl)-2-(tert-butoxy)acetate (intermediate 4)

Clear oil, 0.430 g (8.2%). ¹H NMR (500 MHz, CDCl₃) δ 6.21 (br. s., 1H), 5.03 (spt, J=6.3 Hz, 1H), 4.95 (d, J=15.1 Hz, 1H), 4.64 (dd, J=15.3, 5.0 Hz, 1H), 4.50 (br. s., 1H), 4.05-3.97 (m, 1H), 3.57 (td, J=12.1, 2.5 Hz, 1H), 2.84 (d, J=11.8 Hz, 1H), 2.69 (s, 3H), 2.62 (d, J=11.8 Hz, 1H), 1.66-1.55 (m, 2H), 1.47 (dd, J=13.2, 2.0 Hz, 1H), 1.40-1.34 (m, 1H), 1.23 (d, J=6.3 Hz, 3H), 1.22 (s, 9H), 1.16 (d, J=6.1 Hz, 3H), 1.09 (s, 3H), 1.05 (s, 3H). LCMS (M+H)=485.2 and 487.05.

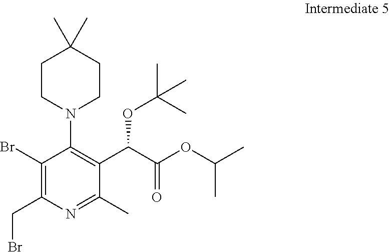 [SMILES: Cc1nc(CBr)c(Br)c(N2CCC(C)(C)CC2)c1[C@H](OC(C)(C)C)C(=O)OC(C)C]

### (S)-Isopropyl 2-(5-bromo-6-(bromomethyl)-4-(4,4-dimethylpiperidin-1-yl)-2-methylpyridin-3-yl)-2-(tert-butoxy)acetate

To a solution of (S)-isopropyl 2-(5-bromo-4-(4,4-dimethylpiperidin-1-yl)-6-(hydroxymethyl)-2-methylpyridin-3-yl)-2-(tert-butoxy)acetate (500 mg, 1.030 mmol) in CH₂Cl₂ (10 mL) was added CBr₄ (376 mg, 1.133 mmol) followed by Ph₃P (297 mg, 1.133 mmol) and the resulting mixture was stirred at room temp for 16 h. Water (2 mL) was then added and the mixture was extracted with dichloromethane (10 mL), dried (Na₂SO₄), filtered and concentrated. The residue was then purified by Biotage (5-30% EtOAc/hexane) to afford (S)-isopropyl 2-(5-bromo-6-(bromomethyl)-4-(4,4-dimethylpiperidin-1-yl)-2-methylpyridin-3-yl)-2-(tert-butoxy)acetate (350 mg, 0.638 mmol, 62.0% yield) as white solid. ¹H NMR (500 MHz, CDCl₃) δ 6.25 (br. s., 1H), 5.14-4.94 (m, 1H), 4.76 (d, J=9.6 Hz, 1H), 4.69 (d, J=9.6 Hz, 1H), 4.04 (br. s., 1H), 3.51 (t, J=11.9 Hz, 1H), 2.91 (d, J=11.5 Hz, 1H), 2.66 (d, J=12.1 Hz, 1H), 2.58 (s, 3H), 1.68-1.55 (m, 2H), 1.47 (d, J=12.5 Hz, 1H), 1.37 (d, J=12.8 Hz, 1H), 1.26-1.23 (m, 3H), 1.22 (s, 9H), 1.16 (d, J=6.1 Hz, 3H), 1.09 (s, 3H), 1.04 (s, 3H). LCMS (M+2H)=549.2.

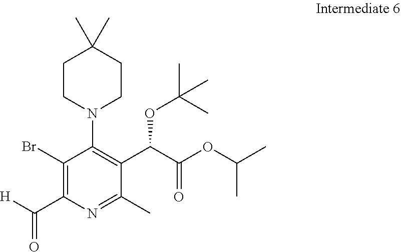 [SMILES: Cc1nc(C=O)c(Br)c(N2CCC(C)(C)CC2)c1[C@H](OC(C)(C)C)C(=O)OC(C)C]

### (S)-Isopropyl 2-(5-bromo-4-(4,4-dimethylpiperidin-1-yl)-6-formyl-2-methylpyridin-3-yl)-2-(tert-butoxy)acetate

To a stirred solution of (S)-isopropyl 2-(5-bromo-4-(4,4-dimethylpiperidin-1-yl)-6-(hydroxymethyl)-2-methylpyridin-3-yl)-2-(tert-butoxy)acetate (15.4 g, 31.7 mmol) in DCM (288 ml) and acetonitrile (28.8 ml) was added Dess-Martin Periodinane (16.15 g, 38.1 mmol) at once at rt. After 5 h, the reaction mixture was diluted with ether (250 mL), washed with 1M NaOH (2×100 ml), brine (200 mL), dried (MgSO₄), filtered and concentrated to afford (S)-isopropyl 2-(5-bromo-4-(4,4-dimethylpiperidin-1-yl)-6-formyl-2-methylpyridin-3-yl)-2-(tert-butoxy)acetate (15.3 g, 31.6 mmol, 99% yield) as yellow solid. ¹H NMR (500 MHz, CDCl₃) δ 10.30 (s, 1H), 6.27 (br s, 1H), 5.13-5.03 (m, 1H), 4.12 (br s, 1H), 3.61-3.52 (m, 1H), 2.96 (br d, J=9.8 Hz, 1H), 2.76-2.70 (m, 1H), 2.66 (s, 3H), 1.66-1.54 (m, 4H), 1.23-1.21 (m, 12H), 1.17 (d, J=6.1 Hz, 3H), 1.11 (s, 3H), 1.06 (s, 3H). LCMS (M+1)=483.1 and 485.1.

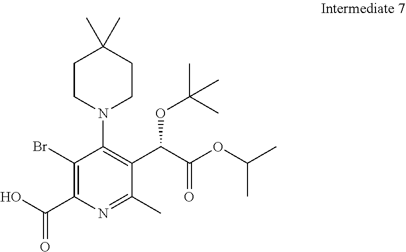 [SMILES: Cc1nc(C(=O)O)c(Br)c(N2CCC(C)(C)CC2)c1[C@H](OC(C)(C)C)C(=O)OC(C)C]

### (S)-3-Bromo-5-(1-(tert-butoxy)-2-isopropoxy-2-oxoethyl)-4-(4,4-dimethylpiperidin-1-yl)-6-methylpicolinic acid

To a solution of (S)-isopropyl 2-(5-bromo-4-(4,4-dimethylpiperidin-1-yl)-6-formyl-2-methylpyridin-3-yl)-2-(tert-butoxy)acetate (15.3 g, 31.6 mmol) in acetonitrile (127 ml) and water (31.6 ml) was added oxone (14.79 g, 24.05 mmol) and the mixture was stirred at RT for 1 hr. The reaction was diluted with water and EtOAc. The organic layer was washed with water (2×) and brine, dried (MgSO₄), filtered and concentrated to afford the product (S)-3-bromo-5-(1-(tert-butoxy)-2-isopropoxy-2-oxoethyl)-4-(4,4-dimethylpiperidin-1-yl)-6-methylpicolinic acid (15.6 g, 31.2 mmol, 99% yield) as a yellow crispy foam. ¹HNMR (500 MHz, CDCl₃) δ 6.33-6.17 (m, 1H), 5.09 (dt, J=12.5, 6.3 Hz, 1H), 4.26-4.14 (m, 1H), 3.67-3.49 (m, 1H), 3.03-2.83 (m, 1H), 2.77-2.65 (m, 1H), 2.62 (s, 3H), 1.64-1.35 (m, 4H), 1.24 (d, J=6.3 Hz, 3H), 1.22 (s, 9H), 1.18 (d, J=6.1 Hz, 3H), 1.10-1.06 (m, 6H). LCMS (M+1)=499.1 and 501.1.

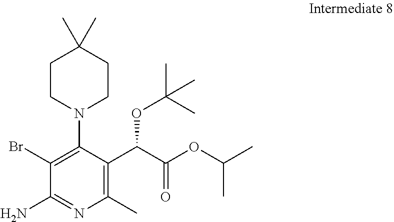 [SMILES: Cc1nc(N)c(Br)c(N2CCC(C)(C)CC2)c1[C@H](OC(C)(C)C)C(=O)OC(C)C]

### Isopropyl (S)-2-(6-amino-5-bromo-4-(4,4-dimethylpiperidin-1-yl)-2-methylpyridin-3-yl)-2-(tert-butoxy)acetate

To a solution of ((S)-3-bromo-5-(1-(tert-butoxy)-2-isopropoxy-2-oxoethyl)-4-(4,4-dimethylpiperidin-1-yl)-6-methylpicolinic acid (6.5 g, 13.01 mmol) and triethylamine (3.63 ml, 26.0 mmol) in toluene (130 mL) was added water (1.172 ml, 65.1 mmol) followed by diphenyl phosphorazidate (5.78 ml, 26.0 mmol). The resulting mixture was heated at 90° C. for 2 h. The reaction mixture was then cooled to ambient temperature, diluted with EtOAc (200 mL), and washed with saturated aqueous NaHCO₃, water, and brine. The organic layer was dried (Na₂SO₄), filtered, and concentrated in vacuo. The residue was then purified on silica gel (220 g column) using 5-80% EtOAc/hexane. The desired fractions were concentrated in vacuo to afford (S)-isopropyl 2-(6-amino-5-bromo-4-(4,4-dimethylpiperidin-1-yl)-2-methylpyridin-3-yl)-2-(tert-butoxy)acetate (5.6 g, 91%) as an off white solid. ¹H NMR (500 MHz, CDCl₃) δ 5.57 (s, 1H), 4.90 (spt, J=6.2 Hz, 1H), 3.04 (dd, J=12.9, 3.0 Hz, 1H), 3.04 (dd, J=12.9, 3.0 Hz, 1H), 3.04 (ddd, J=12.9, 11.7, 3.0 Hz, 1H), 3.04 (ddd, J=12.9, 11.7, 3.0 Hz, 1H), 2.57 (s, 3H), 1.45 (ddd, J=14.2, 3.0, 2.7 Hz, 1H), 1.45 (ddd, J=14.2, 3.0, 2.7 Hz, 1H), 1.45 (ddd, J=14.2, 11.7, 3.0 Hz, 1H), 1.45 (ddd, J=14.2, 11.7, 3.0 Hz, 1H), 1.10 (s, 9H), 1.09 (d, J=6.2 Hz, 6H), 0.98 (s, 3H), 0.91 (s, 3H). LCMS (M+1)=470.10.

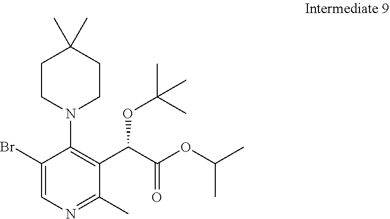 [SMILES: Cc1ncc(Br)c(N2CCC(C)(C)CC2)c1[C@H](OC(C)(C)C)C(=O)OC(C)C]

### (S)-Isopropyl 2-(5-bromo-4-(4,4-dimethylpiperidin-1-yl)-2-methylpyridin-3-yl)-2-(tert-butoxy)acetate

Water (2.81 ml, 156 mmol) followed by acetic acid (4.65 ml, 81 mmol) was added to a stirring solution of (S)-3-bromo-5-(1-(tert-butoxy)-2-isopropoxy-2-oxoethyl)-4-(4,4-dimethylpiperidin-1-yl)-6-methylpicolinic acid (15.6 g, 31.2 mmol) in toluene (156 ml) at rt. The reaction was stirred at 90° C. for 7 hrs. The reaction volatiles were evaporated and the crude material purified via silica gel (330 g column, 5-20% EtOAc:Hex) to afford the product (S)-isopropyl 2-(5-bromo-4-(4,4-dimethylpiperidin-1-yl)-2-methylpyridin-3-yl)-2-(tert-butoxy)acetate (12.8 g, 28.1 mmol, 90% yield) as a clear oil that later crystallized. ¹H NMR (500 MHz, CDCl₃) δ 8.46 (s, 1H), 6.35-6.17 (m, 1H), 5.06 (dt, J=12.5, 6.2 Hz, 1H), 4.10-3.96 (m, 1H), 3.45 (br s, 1H), 2.92 (br s, 1H), 2.72-2.62 (m, 1H), 2.57 (s, 3H), 1.58-1.28 (m, 4H), 1.22 (s, 12H), 1.15 (d, J=6.3 Hz, 3H), 1.07 (br s, 6H). LCMS (M+1)=455.3 and 457.3.

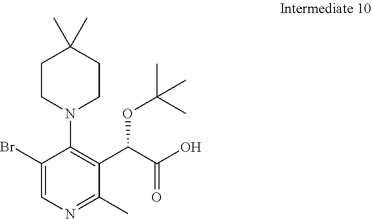 [SMILES: Cc1ncc(Br)c(N2CCC(C)(C)CC2)c1[C@H](OC(C)(C)C)C(=O)O]

### (S)-2-(5-Bromo-4-(4,4-dimethylpiperidin-1-yl)-2-methylpyridin-3-yl)-2-(tert-butoxy)acetic acid

To a solution of (S)-isopropyl 2-(5-bromo-4-(4,4-dimethylpiperidin-1-yl)-2-methylpyridin-3-yl)-2-(tert-butoxy)acetate (2 g, 4.39 mmol) in EtOH (13.17 ml) and water (1.464 ml) was added lithium hydroxide monohydrate (0.590 g, 14.05 mmol) and heated at 75° C. for 18 hrs. The reaction was cooled to RT, neutralized and made slightly acidic with the addition of HCl (15.37 ml, 15.37 mmol). The mixture was extracted with EtOAc. The organic layer was washed with brine, collected, dried over MgSO₄, filtered and the volatiles evaporated to afford the product (S)-2-(5-bromo-4-(4,4-dimethylpiperidin-1-yl)-2-methylpyridin-3-yl)-2-(tert-butoxy)acetic acid (1.82 g, 4.40 mmol, 100% yield) as a white solid. LCMS (M+1)=413.1 and 415.0.

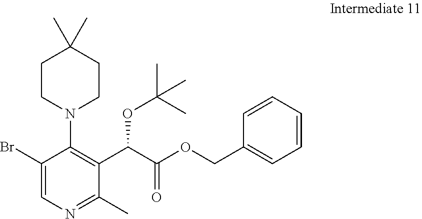 [SMILES: Cc1ncc(Br)c(N2CCC(C)(C)CC2)c1[C@H](OC(C)(C)C)C(=O)OCc1ccccc1]

### (S)-Benzyl 2-(5-bromo-4-(4,4-dimethylpiperidin-1-yl)-2-methylpyridin-3-yl)-2-(tert-butoxy)acetate

To a stirred suspension of (S)-2-(5-bromo-4-(4,4-dimethylpiperidin-1-yl)-2-methylpyridin-3-yl)-2-(tert-butoxy)acetic acid (1.83 g, 4.43 mmol) and cesium carbonate (1.442 g, 4.43 mmol) in anhydrous acetonitrile (14.76 ml) and DMF (7.38 ml) was added benzyl bromide (0.553 ml, 4.65 mmol). The reaction was stirred for 1 hr. After 1 hr, the LCMS indicated the reaction was complete. The reaction was diluted with water and EtOAc. The organic layer was washed with water (2×), followed by brine, collected, dried over MgSO₄, filtered and the volatiles evaporated to afford the crude product. The crude product was purified on silica gel (40 g column, 5-20% EtOAc:Hex) to afford the product (S)-benzyl 2-(5-bromo-4-(4,4-dimethylpiperidin-1-yl)-2-methylpyridin-3-yl)-2-(tert-butoxy)acetate (2.19 g, 4.35 mmol, 98% yield) as a clear oil that solidified upon sitting. ¹H NMR (500 MHz, CDCl₃) δ 8.46 (s, 1H), 7.38-7.31 (m, 3H), 7.27-7.22 (m, 2H), 6.25 (br s, 1H), 5.24-5.04 (m, 2H), 4.07-3.90 (m, 1H), 3.34 (br d, J=2.7 Hz, 1H), 2.76 (br d, J=2.8 Hz, 1H), 2.69-2.57 (m, 1H), 2.52 (s, 3H), 1.60-1.39 (m, 4H), 1.22 (s, 9H), 1.02 (br s, 6H). LCMS (M+1)=503.2 and 505.2.

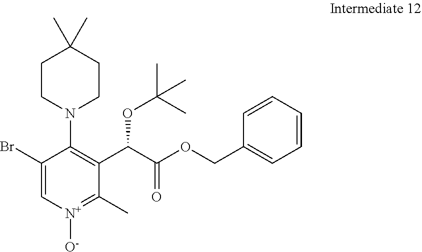 [SMILES: Cc1c([C@H](OC(C)(C)C)C(=O)OCc2ccccc2)c(N2CCC(C)(C)CC2)c(Br)c[n+]1[O-]]

### (S)-3-(2-(Benzyloxy)-1-(tert-butoxy)-2-oxoethyl)-5-bromo-4-(4,4-dimethylpiperidin-1-yl)-2-methylpyridine 1-oxide

To a stirred solution of (S)-benzyl 2-(5-bromo-4-(4,4-dimethylpiperidin-1-yl)-2-methylpyridin-3-yl)-2-(tert-butoxy)acetate (2.19 g, 4.35 mmol) in DCM (40 mL) was added 77% mCPBA (1.462 g, 6.52 mmol) at rt over 5 min. After 4 h, the reaction mixture was washed with 1M NaOH (2×25 mL), dried (MgSO₄), filtered and concentrated to afford the product (S)-3-(2-(benzyloxy)-1-(tert-butoxy)-2-oxoethyl)-5-bromo-4-(4,4-dimethylpiperidin-1-yl)-2-methylpyridine 1-oxide (2.12 g, 4.08 mmol, 94% yield). ¹H NMR (500 MHz, CDCl₃) δ 8.39 (s, 1H), 7.36-7.33 (m, 3H), 7.28-7.25 (m, 2H), 6.26 (br s, 1H), 5.25-5.05 (m, 2H), 3.97-3.88 (m, 1H), 3.33 (brt, J=11.7 Hz, 1H), 2.69 (br d, J=12.9 Hz, 1H), 2.60 (br d, J=9.9 Hz, 1H), 2.48 (s, 3H), 1.58-1.38 (m, 4H), 1.21 (s, 9H), 1.05-0.98 (m, 6H). LCMS (M+1)=519.1 and 521.1.

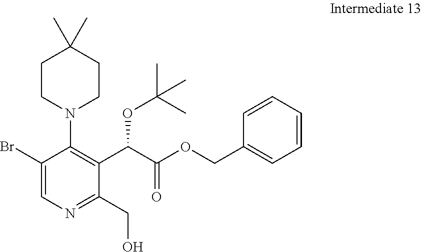 [SMILES: CC1(C)CCN(c2c(Br)cnc(CO)c2[C@H](OC(C)(C)C)C(=O)OCc2ccccc2)CC1]

### (S)-Benzyl 2-(5-bromo-4-(4,4-dimethylpiperidin-1-yl)-2-(hydroxymethyl)pyridin-3-yl)-2-(tert-butoxy)acetate

To a stirred solution of (S)-3-(2-(benzyloxy)-1-(tert-butoxy)-2-oxoethyl)-5-bromo-4-(4,4-dimethylpiperidin-1-yl)-2-methylpyridine 1-oxide (2.12 g, 4.08 mmol) in anhydrous DCM (18.55 ml) was added trifluoroacetic anhydride (0.865 ml, 6.12 mmol) at RT. After 3 h, sat Na₂CO₃ (100 mL) was added and stirred vigorously for 60 minutes. The solution phases were separated and organic phase collected and volatiles evaporated. The residue was taken up in EtOAc and washed with 50 mL of 1 M HCl followed by a wash with sat. sodium carbonate. The organic layer was then washed with brine, collected and volatiles evaporated to give the crude orange oil. The crude product was purified via silica gel (40 g column, 5-20% EtOAc:Hex) to afford the product (S)-benzyl 2-(5-bromo-4-(4,4-dimethylpiperidin-1-yl)-2-(hydroxymethyl)pyridin-3-yl)-2-(tert-butoxy)acetate (1.67 g, 3.21 mmol, 79% yield) as a yellow oil. ¹H NMR (500 MHz, CDCl₃) δ 8.55 (s, 1H), 7.35-7.31 (m, 3H), 7.27-7.23 (m, 2H), 6.27 (s, 1H), 5.23-5.03 (m, 2H), 4.98-4.61 (m, 2H), 4.00-3.86 (m, 1H), 3.45-3.32 (m, 1H), 2.75-2.52 (m, 2H), 1.55-1.36 (m, 4H), 1.22 (s, 9H), 1.01 (br s, 6H). LCMS (M+1)=519.1 and 521.1.

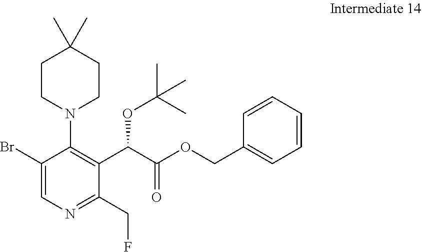 [SMILES: CC1(C)CCN(c2c(Br)cnc(CF)c2[C@H](OC(C)(C)C)C(=O)OCc2ccccc2)CC1]

### (S)-Benzyl 2-(5-bromo-4-(4,4-dimethylpiperidin-1-yl)-2-(fluoromethyl)pyridin-3-yl)-2-(tert-butoxy)acetate

To a stirred solution of (S)-benzyl 2-(5-bromo-4-(4,4-dimethylpiperidin-1-yl)-2-(hydroxymethyl)pyridin-3-yl)-2-(tert-butoxy)acetate (0.2 g, 0.385 mmol) in DCM (3.85 ml) was added Deoxofluor (0.284 ml, 1.540 mmol) dropwise at 0° C. and allowed to stir at 0° C. for 1.5 hr. After 1.5 h, the reaction mixture was diluted with ether (25 mL), washed with 1M potassium phosphate buffer (50 ml), brine (20 mL), dried (MgSO₄), filtered and concentrated to give a purple oil. The crude material was purified via silica gel (24 g column, 5-50% EtOAc:Hex) to afford the product (S)-benzyl 2-(5-bromo-4-(4,4-dimethylpiperidin-1-yl)-2-(fluoromethyl)pyridin-3-yl)-2-(tert-butoxy)acetate (84 mg, 0.161 mmol, 41.8% yield) as a clear thick oil. ¹H NMR (500 MHz, CDCl₃) δ 8.64 (s, 1H), 7.38-7.31 (m, 3H), 7.28-7.25 (m, 2H), 6.25 (s, 1H), 5.77-5.51 (m, 2H), 5.23-5.06 (m, 2H), 4.00-3.89 (m, 1H), 3.41-3.30 (m, 1H), 2.74-2.56 (m, 2H), 1.55-1.40 (m, 4H), 1.20 (s, 9H), 1.05-0.97 (m, 6H). LCMS (M+1)=521.1 and 523.1.

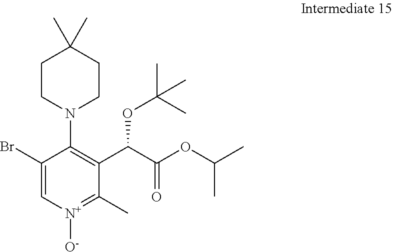 [SMILES: Cc1c([C@H](OC(C)(C)C)C(=O)OC(C)C)c(N2CCC(C)(C)CC2)c(Br)c[n+]1[O-]]

### (S)-5-Bromo-3-(1-(tert-butoxy)-2-isopropoxy-2-oxoethyl)-4-(4,4-dimethylpiperidin-1-yl)-2-methylpyridine 1-oxide

To a stirred solution of (S)-isopropyl 2-(5-bromo-4-(4,4-dimethylpiperidin-1-yl)-2-methylpyridin-3-yl)-2-(tert-butoxy)acetate (0.9 g, 1.976 mmol) in DCM (19.76 ml) was added 77% mCPBA (0.664 g, 2.96 mmol) at rt over 5 min. After 1 h, the reaction mixture was diluted with DCM and washed with sat. Na₂CO₃ (3×25 mL), dried (MgSO₄), filtered and concentrated to afford (S)-5-bromo-3-(1-(tert-butoxy)-2-isopropoxy-2-oxoethyl)-4-(4,4-dimethylpiperidin-1-yl)-2-methylpyridine 1-oxide (873 mg, 1.852 mmol, 94% yield) which was used in the next step without purification. LCMS (M+1)=471.1 and 473.1.

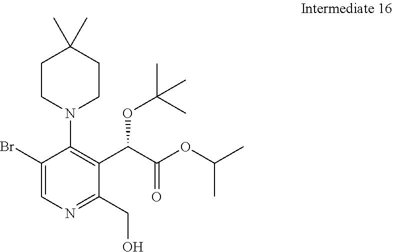 [SMILES: CC(C)OC(=O)[C@@H](OC(C)(C)C)c1c(CO)ncc(Br)c1N1CCC(C)(C)CC1]

### (S)-Isopropyl 2-(5-bromo-4-(4,4-dimethylpiperidin-1-yl)-2-(hydroxymethyl)pyridin-3-yl)-2-(tert-butoxy)acetate

To a stirred solution of (S)-5-bromo-3-(1-(tert-butoxy)-2-isopropoxy-2-oxoethyl)-4-(4,4-dimethylpiperidin-1-yl)-2-methylpyridine 1-oxide (0.873 g, 1.852 mmol) anhydrous DCM (18.52 ml) was added trifluoroacetic anhydride (0.785 ml, 5.56 mmol) at RT. After 3 h, sat. sodium carbonate (50 mL) was added and stirred vigorously for 10 minutes. The solution phases were separated and organic phase collected and volatiles evaporated. The residue was taken up in EtOAc and washed with 50 mL of 1 M HCl followed by a wash with sat. sodium carbonate. The organic layer was then washed with 2 M aq. sodium carbonate (50 mL) for 18 hrs. The crude product was purified via silica gel (40 g column, 5-30% EtOAc:Hex) to afford the product (S)-isopropyl 2-(5-bromo-4-(4,4-dimethylpiperidin-1-yl)-2-(hydroxymethyl)pyridin-3-yl)-2-(tert-butoxy)acetate (720 mg, 1.527 mmol, 82% yield). ¹H NMR (500 MHz, CDCl₃) δ 8.56 (s, 1H), 6.30-6.17 (m, 1H), 5.04 (dt, J=12.5, 6.3 Hz, 1H), 4.97 (d, J=15.3 Hz, 1H), 4.66 (d, J=15.3 Hz, 1H), 4.04-3.94 (m, 1H), 3.58-3.45 (m, 1H), 2.88 (br d, J=3.6 Hz, 1H), 2.71-2.59 (m, 1H), 1.57-1.36 (m, 4H), 1.25-1.22 (m, 12H), 1.16 (d, J=6.3 Hz, 3H), 1.07 (br d, J=12.0 Hz, 6H). LCMS (M+1)=471.1 and 473.1.

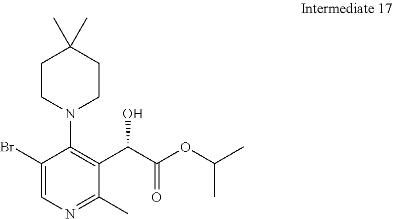 [SMILES: Cc1ncc(Br)c(N2CCC(C)(C)CC2)c1[C@H](O)C(=O)OC(C)C]

### (S)-Isopropyl 2-(5-bromo-4-(4,4-dimethylpiperidin-1-yl)-2-methylpyridin-3-yl)-2-hydroxyacetate

(S)-Isopropyl 2-(5-bromo-4-(4,4-dimethylpiperidin-1-yl)-2-methylpyridin-3-yl)-2-(tert-butoxy)acetate (500 mg, 0.621 mmol) was taken up in a 2:1 solution of TFA/DCE (0.15 M). The reaction was stirred for 2 days and then the mixture was diluted with EtOAc and washed with sat Na₂CO₃. The organic phase was dried over Na₂SO₄, filtered and concentrated, adsorbed onto celite and was purified on silica gel (Biotage, EtOAc/hexanes gradient, 0-100% over 10 CVs) to give the expected product (S)-isopropyl 2-(5-bromo-4-(4,4-dimethylpiperidin-1-yl)-2-methylpyridin-3-yl)-2-hydroxyacetate (450 mg, quant yield). LCMS (M+H)=399 and 401.

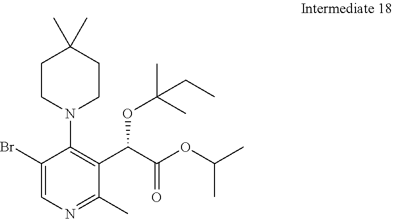 [SMILES: CCC(C)(C)O[C@H](C(=O)OC(C)C)c1c(C)ncc(Br)c1N1CCC(C)(C)CC1]

### (S)-Isopropyl 2-(5-bromo-4-(4,4-dimethylpiperidin-1-yl)-2-methylpyridin-3-yl)-2-(tert-pentyloxy)acetate

In a 20 mL microwave vial, (S)-isopropyl 2-(5-bromo-4-(4,4-dimethylpiperidin-1-yl)-2-methylpyridin-3-yl)-2-hydroxyacetate (440 mg, 1.10 mmol), CH₂Cl₂ (9.1 ml), and 2-methylbut-2-ene (3.7 ml, 44 mmol) were combined. Then, the vial was capped and perchloric acid (0.284 ml, 3.31 mmol) was added at once to the reaction (see ppt form and then go back into a clear solution). The reaction was stirred for 4 hrs. The mixture was diluted with EtOAc and washed with sat Na₂CO₃. The organic phase was dried over Na₂SO₄, filtered and concentrated, adsorbed onto celite and was purified on silica gel (Biotage, EtOAc/hexanes gradient, 0-100% over 10 CVs) to give 112 mg of (S)-isopropyl 2-(5-bromo-4-(4,4-dimethylpiperidin-1-yl)-2-methylpyridin-3-yl)-2-(tert-pentyloxy)acetate (21% yield). LCMS (M+H)=469, and 471. Also 297 mg of starting material was recovered.

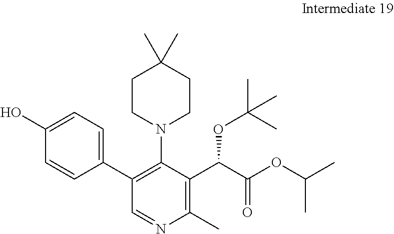 [SMILES: Cc1ncc(-c2ccc(O)cc2)c(N2CCC(C)(C)CC2)c1[C@H](OC(C)(C)C)C(=O)OC(C)C]

### (S)-Isopropyl 2-(tert-butoxy)-2-(4-(4,4-dimethylpiperidin-1-yl)-5-(4-hydroxyphenyl)-2-methylpyridin-3-yl)acetate

(S)-Isopropyl 2-(5-bromo-4-(4,4-dimethylpiperidin-1-yl)-2-methylpyridin-3-yl)-2-(tert-butoxy)acetate (100 mg, 0.220 mmol), (4-hydroxyphenyl)boronic acid (36 mg, 0.26 mmol), chloro(2-dicyclohexylphosphino-2′,6′-dimethoxy-1,1′-biphenyl) [2-(2′-amino-1,1′-biphenyl)]palladium(II) (16 mg, 0.022 mmol), potassium phosphate tribasic (140 mg, 0.659 mmol) were combined under N₂ (g). 1,4-Dioxane (3.7 ml) and water (0.7 ml) was added under N₂ (g). The reaction was stirred at 80° C. for 1 hr. The reaction was concentrated, adsorbed onto celite and was purified on silica gel (Biotage, EtOAc/hexanes gradient, 0-100% over 10 CVs) to give the expected product (S)-isopropyl 2-(tert-butoxy)-2-(4-(4,4-dimethylpiperidin-1-yl)-5-(4-hydroxyphenyl)-2-methylpyridin-3-yl)acetate (75 mg, 0.160 mmol, 72.9% yield). LCMS (M+H)=469.25.

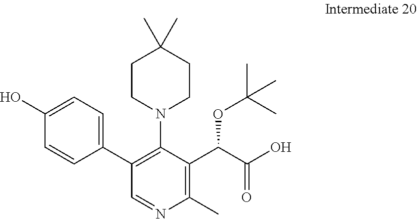 [SMILES: Cc1ncc(-c2ccc(O)cc2)c(N2CCC(C)(C)CC2)c1[C@H](OC(C)(C)C)C(=O)O]

### (S)-2-(tert-Butoxy)-2-(4-(4,4-dimethylpiperidin-1-yl)-5-(4-hydroxyphenyl)-2-methylpyridin-3-yl)acetic acid

5N NaOH (0.2 ml, 1 mmol) was added to a stirring solution of (S)-isopropyl 2-(tert-butoxy)-2-(4-(4,4-dimethylpiperidin-1-yl)-5-(4-hydroxyphenyl)-2-methylpyridin-3-yl)acetate (50 mg, 0.11 mmol) in EtOH (2 ml) at 80° C. The reaction was stirred overnight and then purified by preparative reverse phase HPLC to afford (S)-2-(tert-butoxy)-2-(4-(4,4-dimethylpiperidin-1-yl)-5-(4-hydroxyphenyl)-2-methylpyridin-3-yl)acetic acid (20 mg, 0.047 mmol, 43.9% yield). LCMS (M+H)=427.15.

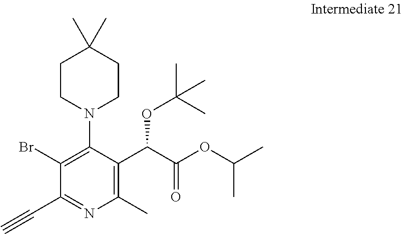 [SMILES: C#Cc1nc(C)c([C@H](OC(C)(C)C)C(=O)OC(C)C)c(N2CCC(C)(C)CC2)c1Br]

### (S)-Isopropyl 2-(5-bromo-4-(4,4-dimethylpiperidin-1-yl)-6-ethynyl-2-methylpyridin-3-yl)-2-(tert-butoxy)acetate

Dimethyl (2-diazo-3-oxobutanoyl)phosphonite (507 mg, 2.48 mmol) was added to a stirring solution of (S)-isopropyl 2-(5-bromo-4-(4,4-dimethylpiperidin-1-yl)-6-formyl-2-methylpyridin-3-yl)-2-(tert-butoxy)acetate (1.0 g, 2.1 mmol) and K₂CO₃ (1.3 g, 9.3 mmol) in MeOH (21 ml) at rt. The reaction was stirred for 1 hr. The reaction was then concentrated, adsorbed onto celite and was purified on silica gel (Biotage, EtOAc/hexanes gradient) to afford (S)-isopropyl 2-(5-bromo-4-(4,4-dimethylpiperidin-1-yl)-6-ethynyl-2-methylpyridin-3-yl)-2-(tert-butoxy)acetate (923 mg, 1.925 mmol, 93% yield) was isolated. LCMS (M+H)=479.10 and 481.05.

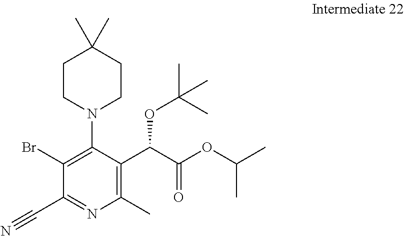 [SMILES: Cc1nc(C#N)c(Br)c(N2CCC(C)(C)CC2)c1[C@H](OC(C)(C)C)C(=O)OC(C)C]

### (S)-Isopropyl 2-(5-bromo-6-cyano-4-(4,4-dimethylpiperidin-1-yl)-2-methylpyridin-3-yl)-2-(tert-butoxy)acetate

Under N₂, tert-butyl nitrite (25 μl, 0.21 mmol) was added to a stirring solution of (S)-isopropyl 2-(5-bromo-4-(4,4-dimethylpiperidin-1-yl)-6-ethynyl-2-methylpyridin-3-yl)-2-(tert-butoxy)acetate (50 mg, 0.10 mmol) and 2-methylpyridine 1-oxide (23 mg, 0.209 mmol) in THF (1 ml) at 70° C. under N₂. The reaction was stirred overnight at 70° C. LCMS showed ˜1:1 mixture of sm and product. The reaction was concentrated, adsorbed onto celite and was purified on silica gel (Biotage, EtOAc/hexanes gradient) to afford (S)-isopropyl 2-(5-bromo-6-cyano-4-(4,4-dimethylpiperidin-1-yl)-2-methylpyridin-3-yl)-2-(tert-butoxy)acetate (18 mg, 0.037 mmol, 35.9% yield). LCMS (M+H)=480.05 and 482.10.

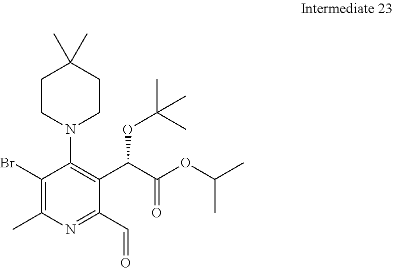 [SMILES: Cc1nc(C=O)c([C@H](OC(C)(C)C)C(=O)OC(C)C)c(N2CCC(C)(C)CC2)c1Br]

### (S)-Isopropyl 2-(5-bromo-4-(4,4-dimethylpiperidin-1-yl)-2-formyl-6-methylpyridin-3-yl)-2-(tert-butoxy)acetate

To a stirred solution of (S)-isopropyl 2-(5-bromo-4-(4,4-dimethylpiperidin-1-yl)-2-(hydroxymethyl)-6-methylpyridin-3-yl)-2-(tert-butoxy)acetate (300 mg, 0.618 mmol) in DCM (5618 μl) and acetonitrile (562 μl) was added Dess-Martin Periodinane (393 mg, 0.927 mmol) at once at rt. After 5 h, the reaction mixture was diluted with ether (25 mL), washed with 1M NaOH (2×20 ml), brine (5 mL), dried (MgSO₄), filtered and concentrated to afford the product (S)-isopropyl 2-(5-bromo-4-(4,4-dimethylpiperidin-1-yl)-2-formyl-6-methylpyridin-3-yl)-2-(tert-butoxy)acetate (285 mg, 0.590 mmol, 95% yield). ¹H NMR (500 MHz, CDCl₃) δ 10.57-10.25 (m, 1H), 6.39-6.19 (m, 1H), 5.10-5.02 (m, 1H), 3.96-3.84 (m, 1H), 3.51 (brt, J=10.2 Hz, 1H), 2.91 (br s, 1H), 2.78 (s, 3H), 2.68-2.63 (m, 1H), 1.49-1.37 (m, 2H), 1.36-1.27 (m, 2H), 1.26 (d, J=6.3 Hz, 3H), 1.20-1.18 (m, 12H), 1.07 (br d, J=14.3 Hz, 6H). LCMS (M+1) 483.1 and 485.1.

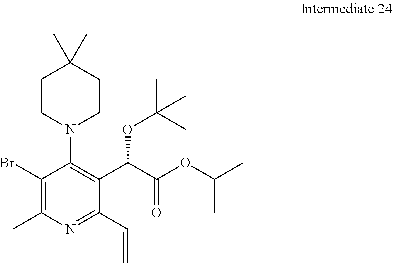 [SMILES: C=Cc1nc(C)c(Br)c(N2CCC(C)(C)CC2)c1[C@H](OC(C)(C)C)C(=O)OC(C)C]

### (S)-Isopropyl 2-(5-bromo-4-(4,4-dimethylpiperidin-1-yl)-6-methyl-2-vinylpyridin-3-yl)-2-(tert-butoxy)acetate

To a suspension of methyltriphenylphosphonium bromide (111 mg, 0.310 mmol) in THF (1 ml) at 0° C. was added sodium hydride (12.41 mg, 0.310 mmol) and the resulting mixture was stirred at rt for 45 min. (S)-Isopropyl 2-(5-bromo-4-(4,4-dimethylpiperidin-1-yl)-2-formyl-6-methylpyridin-3-yl)-2-(tert-butoxy)acetate (50 mg, 0.103 mmol) dissolved in THF (1 ml) was added dropwise and the mixture was stirred at 0° C. for 1 h then warmed to rt and stirred 18 h. The reaction was quenched with water and the product was extracted with EtOAc. The organic phase was washed with brine, dried (MgSO₄), filtered and concentrated. The residue was purified by silica gel chromatography (12 g column; 5-20% EtOAc/hexane) to afford (S)-isopropyl 2-(5-bromo-4-(4,4-dimethylpiperidin-1-yl)-6-methyl-2-vinylpyridin-3-yl)-2-(tert-butoxy)acetate (25 mg, 0.052 mmol, 50.2% yield). ¹H NMR (500 MHz, CDCl₃) δ 7.43-7.33 (m, 1H), 6.34 (br dd, J=16.8, 2.3 Hz, 2H), 5.46-5.38 (m, 1H), 5.07-4.98 (m, 1H), 4.07 (br s, 1H), 3.58-3.49 (m, 1H), 2.91 (br d, J=11.2 Hz, 1H), 2.69 (s, 3H), 2.63 (br d, J=12.6 Hz, 1H), 1.67-1.53 (m, 4H), 1.22 (s, 9H), 1.20 (d, J=6.3 Hz, 3H), 1.11-1.09 (m, 6H), 1.04 (s, 3H). LCMS (M+1)=481.2 and 483.1.

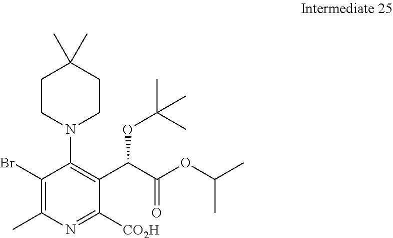 [SMILES: Cc1nc(C(=O)O)c([C@H](OC(C)(C)C)C(=O)OC(C)C)c(N2CCC(C)(C)CC2)c1Br]

### (S)-5-Bromo-3-(1-(tert-butoxy)-2-isopropoxy-2-oxoethyl)-4-(4,4-dimethylpiperidin-1-yl)-6-methylpicolinic acid

To a solution of (S)-isopropyl 2-(5-bromo-4-(4,4-dimethylpiperidin-1-yl)-2-formyl-6-methylpyridin-3-yl)-2-(tert-butoxy)acetate (0.350 g, 0.724 mmol) in DMSO (10 ml) was added potassium phosphate monobasic (0.296 g, 2.172 mmol) in water (0.5 mL) followed by sodium chlorite (0.196 g, 2.172 mmol) in water (1.0 mL) and the mixture was stirred for 3 hr. The reaction was diluted with water and EtOAc. The organic layer was washed with water (2×). The organic layer was then washed with brine dried (MgSO₄), filtered and concentrated to afford the product (S)-5-bromo-3-(1-(tert-butoxy)-2-isopropoxy-2-oxoethyl)-4-(4,4-dimethylpiperidin-1-yl)-6-methylpicolinic acid (319 mg, 0.639 mmol, 88% yield) as a sticky oil. ¹H NMR (500 MHz, CDCl₃) δ 7.18-6.63 (m, 1H), 5.12-4.99 (m, 1H), 3.58-3.48 (m, 1H), 2.99-2.91 (m, 1H), 2.74 (s, 3H), 2.64 (s, 1H), 1.48-1.33 (m, 4H), 1.25 (d, J=6.3 Hz, 3H), 1.21 (s, 9H), 1.18 (d, J=6.3 Hz, 3H), 1.03 (s, 6H). LCMS (M+1)=500.0 and 501.0.

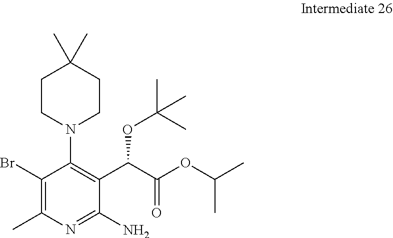 [SMILES: Cc1nc(N)c([C@H](OC(C)(C)C)C(=O)OC(C)C)c(N2CCC(C)(C)CC2)c1Br]

Water (57.5 μl, 3.19 mmol) followed by diphenylphosphoryl azide (276 μl, 1.277 mmol) was added to a stirring solution of (S)-5-bromo-3-(1-(tert-butoxy)-2-isopropoxy-2-oxoethyl)-4-(4,4-dimethylpiperidin-1-yl)-6-methylpicolinic acid (319 mg, 0.639 mmol) and TEA (178 μl, 1.277 mmol) in Toluene (6387 μl) at rt. The reaction was stirred at 90° C. for 2 hrs. The mixture was then cooled to room temp, diluted with EtOAc (100 mL) and washed with sat. NaHCO₃ solution. The organic layer was then washed with brine, collected, dried (MgSO₄), filtered and concentrated. The residue was then purified by silica gel (40 g column, 5-50% EtOAc:hexane) to afford (S)-isopropyl 2-(2-amino-5-bromo-4-(4,4-dimethylpiperidin-1-yl)-6-methylpyridin-3-yl)-2-(tert-butoxy)acetate (150 mg, 0.319 mmol, 49.9% yield) as a yellow solid. ¹H NMR (500 MHz, CDCl₃) δ 6.21 (s, 1H), 5.36-5.19 (m, 2H), 5.02 (dt, J=12.6, 6.2 Hz, 1H), 3.98 (td, J=12.1, 2.6 Hz, 1H), 3.60 (td, J=12.0, 2.4 Hz, 1H), 2.85-2.82 (m, 1H), 2.62-2.56 (m, 1H), 2.49 (s, 3H), 1.66-1.58 (m, 2H), 1.44 (br dd, J=12.8, 2.4 Hz, 1H), 1.39-1.35 (m, 1H), 1.25 (s, 9H), 1.23 (d, J=6.3 Hz, 3H), 1.14 (d, J=6.3 Hz, 3H), 1.09 (s, 3H), 1.04 (s, 3H). LCMS (M+1)=470.1 and 472.1.

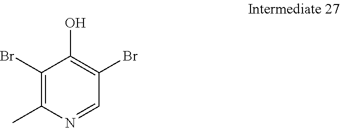

### 3,5-Dibromo-2-methylpyridin-4-ol

To a stirred solution of 2-methylpyridin-4-ol (5 g, 45.8 mmol) in DCM (56.4 ml) and MeOH (6.80 ml) was added tert-butylamine (9.81 ml, 93 mmol) and cooled to 0° C. Bromine (4.72 ml, 92 mmol) was added dropwise over 60 minutes. The reaction mixture was stirred at RT for 3 hours. The reaction mixture was filtered through a fine frit filter and the solid white material dried under vacuum for 18 hrs. ¹H NMR (500 MHz, DMSO-d₆) δ 12.32 (br. s., 1H), 8.21 (s, 1H), 2.40 (s, 3H). LCMS (M+1)=267.7.

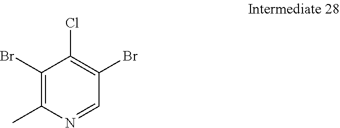

### 3,5-Dibromo-4-chloro-2-methylpyridine

To a solution of 3,5-dibromo-2-methylpyridin-4-ol (13.12 g, 49.2 mmol) in POCl₃ (13.74 ml, 147 mmol) was added triethylamine (6.85 ml, 49.2 mmol) at 0° C. slowly over 80 min. After addition ice bath was removed, and the reaction was heated to 80° C. and stirred for 3 h. The reaction mixture was then cooled to rt and slowly quenched by adding it to crushed ice. The resulting suspension was extracted with DCM (250 ml). The organic layer was washed with saturated NaHCO₃ solution (250 mL) followed by water (250 mL) and brine (250 mL). The organic layer was dried (MgSO₄), filtered and concentrated to get 3,5-dibromo-4-chloro-2-methylpyridine (14.7 g, 51.5 mmol, 105% yield) as a off white solid. ¹H NMR (500 MHz, CDCl₃) δ 8.55 (s, 1H), 2.72 (s, 3H). LCMS (M+1)=285.7.

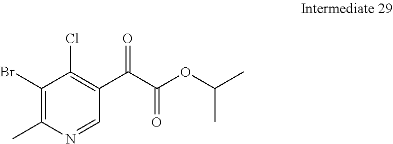 [SMILES: Cc1ncc(C(=O)C(=O)OC(C)C)c(Cl)c1Br]

### Isopropyl 2-(5-bromo-4-chloro-6-methylpyridin-3-yl)-2-oxoacetate

To a −78° C. solution of 3,5-dibromo-4-chloro-2-methylpyridine (9.42 g, 33.0 mmol) and copper(I) bromide-dimethyl sulfide complex (0.339 g, 1.651 mmol) in THF (75 mL) was added dropwise isopropylmagnesium chloride (17.33 mL, 34.7 mmol) over 20 min. The reaction was allowed to warm to −10° C. for 60 min. The reaction mixture was then transferred via cannula to another flask containing a solution of isopropyl 2-chloro-2-oxoacetate (4.97 g, 33.0 mmol) in THF (75 ml) at −60° C. and allowed to warm to −10° C. for 2.5 hr. The reaction was then quenched with 10% solution of ammonium chloride and diethyl ether. The organic layer was washed with brine, collected, dried (MgSO₄), filtered and volatiles evaporated to give the crude material. The crude material was purified via silica gel (330 g column, 10-40% EtOAc:Hex) to give the product isopropyl 2-(5-bromo-4-chloro-6-methylpyridin-3-yl)-2-oxoacetate (3.45 g, 9.15 mmol, 27.7% yield) as a yellow oil that later solidified. ¹H NMR (500 MHz, methanol-d₄) δ 8.79 (s, 1H), 5.09 (dt, J=12.6, 6.2 Hz, 1H), 2.76 (s, 3H), 1.24-1.22 (m, 3H), 1.20 (d, J=6.3 Hz, 3H). LCMS (M+1)=321.8.

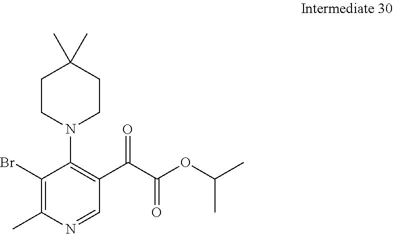 [SMILES: Cc1ncc(C(=O)C(=O)OC(C)C)c(N2CCC(C)(C)CC2)c1Br]

### Isopropyl 2-(5-bromo-4-(4,4-dimethylpiperidin-1-yl)-6-methylpyridin-3-yl)-2-oxoacetate

To a 40 mL vial equipped with a stir bar was added isopropyl 2-(5-bromo-4-chloro-6-methylpyridin-3-yl)-2-oxoacetate (5 g, 15.60 mmol), DIPEA (3.00 ml, 17.16 mmol) and acetonitrile (10.40 ml), then 4,4-dimethylpiperidine (1.942 g, 17.16 mmol). The vial was capped and then placed in a heating block at 85° C. with stirring. After 18 hrs the reaction mixture was dissolved in Et₂O (100 mL) and water (100 mL) and transferred to a 500 mL separatory funnel. The mixture was agitated; the phases were separated. The aq. phase was back extracted with Et₂O (100 mL). The combined organics were washed with brine (50 mL). The solution was dried over MgSO₄; filtered; then concentrated in vacuo. The crude product was purified via silica gel purification (120 g column, 0-30% EtOAc:Hex) to give the product isopropyl 2-(5-bromo-4-(4,4-dimethylpiperidin-1-yl)-6-methylpyridin-3-yl)-2-oxoacetate (4.56 g, 11.48 mmol, 73.6% yield) as a yellow oil that partially solidified. ¹H NMR (500 MHz, CDCl₃) δ 8.45 (s, 1H), 5.26 (dt, J=12.5, 6.3 Hz, 1H), 3.20-3.14 (m, 4H), 2.76 (s, 3H), 1.52-1.48 (m, 4H), 1.42 (d, J=6.3 Hz, 6H), 1.04 (s, 6H). LCMS (M+1)=399.0.

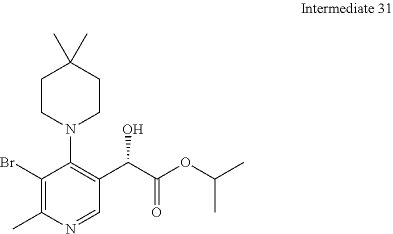 [SMILES: Cc1ncc([C@H](O)C(=O)OC(C)C)c(N2CCC(C)(C)CC2)c1Br]

### (S)-Isopropyl 2-(5-bromo-4-(4,4-dimethylpiperidin-1-yl)-6-methylpyridin-3-yl)-2-hydroxyacetate

To a 100 mL R B-flask equipped with a stir bar was added isopropyl 2-(5-bromo-4-(4,4-dimethylpiperidin-1-yl)-6-methylpyridin-3-yl)-2-oxoacetate (2.5 g, 6.29 mmol). The flask was fitted with a rubber septum and then placed under N₂ atm (vac/fill×3). To the flask was added toluene (17.98 ml). The flask was placed in a −35° C. bath (dichloroethane/dry ice). A thermometer was used to monitor the internal temperature. When the internal temp was −30° C., to the flask was added (R)-1-methyl-3,3-diphenylhexahydropyrrolo[1,2-c][1,3,2]oxazaborole (0.944 ml, 0.944 mmol). To the stirred solution was added 50% catecholborane/toluene (1.886 ml, 8.81 mmol) over 2 minutes. Within 5 minutes following the addition the temperature rose to −25° C. before falling to −30° C. The solution was stirred at −30° C. for 3 h. The flask was transferred to a −15 to −12° C. cold bath (chiller/circulator). The yellow solution was stirred for 1 day at −15 to −12° C. The reaction was quenched with 5 mL of 2M aq. sodium carbonate. The reaction was then diluted with 100 mL EtOAc and 100 mL 2M aq sodium carbonate and stirred vigorously for 2 hrs. The layers were separated and the organic layer collected and stirred vigorously for an additional 1 hr. The organic layer was washed with brine, dried over MgSO₄, filtered and evaporated to give the crude product. The crude product was purified on silica gel chromatography (80 g column, 10-40% EtOAc:Hex) to afford the product (S)-isopropyl 2-(5-bromo-4-(4,4-dimethylpiperidin-1-yl)-6-methylpyridin-3-yl)-2-hydroxyacetate (2 g, 5.01 mmol, 80% yield) as a yellow oil that solidified at RT. ¹H NMR (500 MHz, CDCl₃) δ 8.33 (s, 1H), 5.31 (d, J=6.9 Hz, 1H), 5.13-5.03 (m, 2H), 3.80 (br. s., 2H), 2.87-2.75 (m, 1H), 2.71 (s, 3H), 2.69-2.60 (m, 1H), 1.71-1.59 (m, 2H), 1.43 (d, J=14.8 Hz, 2H), 1.28 (d, J=6.1 Hz, 3H), 1.16 (d, J=6.3 Hz, 3H), 1.04 (s, 3H), 1.08 (s, 3H). LCMS (M+1)=399.0.

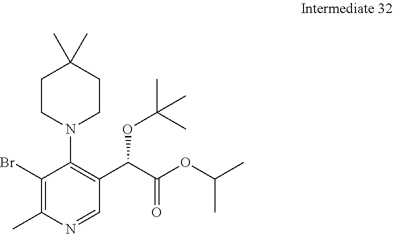 [SMILES: Cc1ncc([C@H](OC(C)(C)C)C(=O)OC(C)C)c(N2CCC(C)(C)CC2)c1Br]

### (S)-Isopropyl 2-(5-bromo-4-(4,4-dimethylpiperidin-1-yl)-6-methylpyridin-3-yl)-2-(tert-butoxy)acetate

In a 250 ml round bottom flask fitted with a shlenk adaptor with rubber septum (with empty balloon attached), isobutylene gas was vigorously bubbled for 30 minutes into a 0° C. solution of (S)-isopropyl 2-(5-bromo-4-(4,4-dimethylpiperidin-1-yl)-6-methylpyridin-3-yl)-2-hydroxyacetate (2 g, 5.01 mmol) and perchloric acid (0.861 mL, 10.02 mmol) in DCM (100 mL) until the volume doubled and the balloon filled to firmness. After 2 hrs, the isobutylene line was disconnected and needle pulled to just above the solution line then connected to a bubbler to monitor isobutylene gas exit. The ice bath was removed and warmed up to RT while monitoring for conversion. After 2 hrs the reaction appeared to go to full conversion according to LCMS. The reaction mixture was poured into a 1 L Erlenmeyer flask and made basic with 2M sodium carbonate while vigorously stirring. The organic layer was separated and washed with water, followed by brine, collected, dried (MgSO₄), filtered and volatiles evaporated to afford the crude product. The crude product was purified on silica gel (40 g column, 5-40% EtOAc:Hex) to afford the product (S)-isopropyl 2-(5-bromo-4-(4,4-dimethylpiperidin-1-yl)-6-methylpyridin-3-yl)-2-(tert-butoxy)acetate (1.95 g, 4.28 mmol, 85% yield) as a clear oil that later crystallized to a white solid. ¹H NMR (500 MHz, CDCl₃) δ 8.61 (s, 1H), 5.61 (s, 1H), 5.01 (dt, J=12.5, 6.3 Hz, 1H), 3.81 (t, J=10.9 Hz, 1H), 3.60 (t, J=11.0 Hz, 1H), 2.76 (d, J=11.5 Hz, 1H), 2.69 (s, 3H), 2.64 (d, J=12.1 Hz, 1H), 1.63-1.51 (m, 2H), 1.46 (d, J=11.2 Hz, 1H), 1.38 (d, J=14.2 Hz, 1H), 1.26-1.22 (m, 12H), 1.20 (d, J=6.1 Hz, 3H), 1.09-1.03 (m, 6H). LCMS (M+1)=457.1.

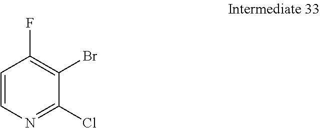

### 3-Bromo-2-chloro-4-fluoropyridine

To a dry 1000 mL round bottom flask under nitrogen was added 2-chloro-4-fluoropyridine (13 g, 99 mmol) and THF (250 mL). The reaction was flushed with argon, securely capped, cooled to −78° C. and slowly (over 20-25 min) treated with LDA, 1 M in THF:hexanes (100 ml, 100 mmol). After the addition was complete, the reaction was stirred at −78° C. for 85 min. The reaction was then treated (over 10 min) with a solution of 1,2-dibromo-1,1,2,2-tetrachloroethane (35 g, 107 mmol) in THF (150 mL). After the addition was complete, the reaction was stirred at −78° C. for 30 min, then the bath was removed and the reaction was allowed to warm to room temp. The reaction was diluted with dichloromethane (500 mL), extracted with water (1×150 mL), brine (1×100 mL), dried over Na₂SO₄, and concentrated. The crude material was purified via silica gel chromatography (330 g SiO₂ column, hexane:dichloromethane 100:0->65:35) to afford 3-bromo-2-chloro-4-fluoropyridine, 12 g (58%). ¹H NMR (500 MHz, CDCl₃) δ 8.33 (dd, J=7.4, 5.4 Hz, 1H), 7.07 (dd, J=7.1, 5.4 Hz, 1H).

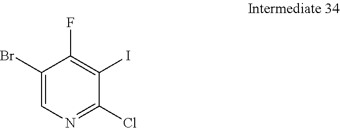

### 5-Bromo-2-chloro-4-fluoro-3-iodopyridine

To a flame dried 500 mL conical flask under nitrogen was added 2,2,6,6-tetramethylpiperidine (12.7 mL, 75 mmol) and THF (225 mL). The reaction was flushed with argon, cooled to −78° C. and treated with n-butyllithium, 1.6M in hexanes (39 mL, 62.4 mmol). The reaction was stirred at −78 C, then the bath was removed and the reaction was allowed to warm to 0° C. over 20 min. The reaction was recooled to −78° C. and transferred to a solution of 3-bromo-2-chloro-4-fluoropyridine (13.55 g, 64.4 mmol) in THF (225 mL) at −78° C. over 10 min. After the addition was complete, the reaction was stirred at −78° C. for 50 min, then treated with a solution of iodine (18.8 g, 74.1 mmol) in THF (225 mL) at −50 C. The reaction was packed in dry ice and allowed to stir while slowly warming to room temp over 18 h. The reaction was diluted with ethyl acetate (600 mL), extracted with aq Na₂S₂O₃ (5 g dissolved in water (250 mL)), water (1×60 mL), brine (1×60 mL), dried over Na₂SO₄, and concentrated. The crude material was purified via silica gel chromatography (330 g SiO₂ column, hexane:dichloromethane: 100:0->65:35) to afford 5-bromo-2-chloro-4-fluoro-3-iodopyridine, 15.7 g (72%). ¹H NMR (500 MHz, CDCl₃) δ 8.42 (d, J=8.4 Hz, 1H)

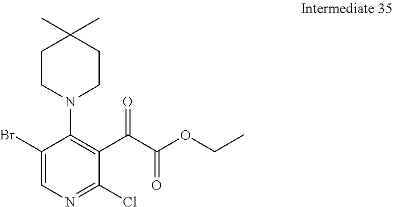 [SMILES: CCOC(=O)C(=O)c1c(Cl)ncc(Br)c1N1CCC(C)(C)CC1]

### Ethyl 2-(5-bromo-2-chloro-4-(4,4-dimethylpiperidin-1-yl)pyridin-3-yl)-2-oxoacetate

To a dry 500 mL conical flask under nitrogen was added 5-bromo-2-chloro-4-fluoro-3-iodopyridine (11.34 g, 33.7 mmol), copper(I) bromide-dimethyl sulfide (1.36 g, 6.62 mmol) and THF (400 mL). The resulting suspension was flushed very well with argon, cooled to −78 C. and treated with isopropylmagnesium chloride, 1.92 M in THF (17.56 mL, 33.7 mmol). After the addition was complete, the reaction was stirred at −78° C. for 33 min, then allowed to warm to −10° C. over 2 h. The reaction was recooled to −60° C. and transferred via cannulae to a solution of ethyl oxalyl chloride (6.34 mL, 56.8 mmol) in THF (100 mL) at −78 C. The reaction was stirred at −78° C. for 5 min, slowly allowed to warm to −12 C over 85 min then held at −12° C. for 2.5 h. The reaction was cooled to −35° C. and treated (via cannulae) with a solution of 4,4-dimethylpiperidine (16 g, 141 mmol) in N,N-diisopropylethylamine (19.2 mL, 110 mmol), followed by acetonitrile (100 mL) and allowed to stir while slowly warming to room temp over 18 h. The reaction was treated with diethanol amine (805 mg, 7.66 mmol), diluted with ethyl acetate (1100 mL), extracted with water (1×150 mL), dried over Na₂SO₄ and concentrated. The crude material was purified via silica gel chromatography (330 g SiO₂ column, hexane:dichloromethane: 100:0->0:100) to afford ethyl 2-(5-bromo-2-chloro-4-(4,4-dimethylpiperidin-1-yl)pyridin-3-yl)-2-oxoacetate, 10.81 g (79%). LCMS (M+1)=403.1 and 405.0.

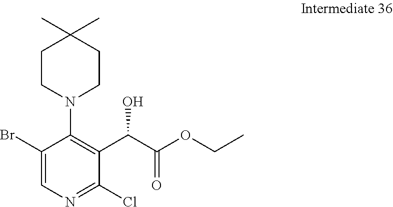 [SMILES: CCOC(=O)[C@@H](O)c1c(Cl)ncc(Br)c1N1CCC(C)(C)CC1]

### Ethyl (S)-2-(5-bromo-2-chloro-4-(4,4-dimethylpiperidin-1-yl)pyridin-3-yl)-2-hydroxyacetate

To a solution of ethyl 2-(5-bromo-2-chloro-4-(4,4-dimethylpiperidin-1-yl)pyridin-3-yl)-2-oxoacetate (11.6 g, 28.7 mmol) in anhydrous Toluene (500 mL) was added (R)-2-methyl-CBS-oxazaborolidine, 1.0M in toluene (35.5 mL, 35.5 mmol). The reaction was cooled to −78° C. and treated (over 33 min) with catecholborane, 50 wt % in toluene (29 mL, 135 mmol). After the addition was complete, the dry ice was removed from the bath and the reaction was allowed to warm to −5° C. over 5 h, then held at −5° C. for 40 min. The reaction was cooled to −55° C. and transferred (via cannulae) to an ice cold solution of aqueous saturated K₂CO₃ (200 mL) and EtOAc (400 mL) and the resulting two phase mixture was stirred at room temp for 18 h. The reaction was further diluted with ethyl acetate, extracted with water (1×30 mL), brine (1×30 mL), dried over Na₂SO₄ and concentrated. The crude material was purified via silica gel chromatography (330 g SiO₂ column, dichloromethane:EtOAc 100:0->50:50) to afford ethyl (S)-2-(5-bromo-2-chloro-4-(4,4-dimethylpiperidin-1-yl)pyridin-3-yl)-2-hydroxyacetate, 11.5 g (99%). LCMS (M+1)=405.0 and 407.0.

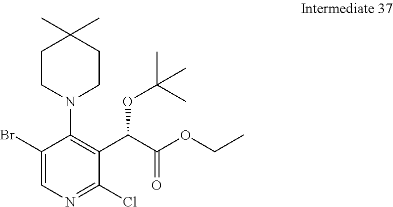 [SMILES: CCOC(=O)[C@@H](OC(C)(C)C)c1c(Cl)ncc(Br)c1N1CCC(C)(C)CC1]

### Ethyl-(S)-2-(5-bromo-2-chloro-4-(4,4-dimethylpiperidin-1-yl)pyridin-3-yl)-2-(tert-butoxy)acetate

To a dry 1000 mL pressure bottle under nitrogen was added (S)-ethyl 2-(5-bromo-2-chloro-4-(4,4-dimethylpiperidin-1-yl)pyridin-3-yl)-2-hydroxyacetate (5.23 g, 12.89 mmol), CH₂Cl₂ (300 mL) and 4 A mol sieves. The reaction was allowed to stir at room temp for 2 h, then cooled to −78° C. The reaction was then treated with perchloric acid (2.33 mL, 38.7 mmol), and isobutylene was bubbled into the reaction until the reaction volume roughly doubled. The flask was securely capped, removed from the bath and allowed to stir for 18 h while slowly warming to room temp. The reaction was cooled to −60° C. and quenched into an erlenmeyer flask containing a mixture of CH₂Cl₂ (200 mL) and NaHCO₃ (12.8 g, 152 mmol) dissolved in water (250 mL). The reaction was further diluted with dichloromethane (300 mL), extracted with water (1×75 mL), brine (1×75 mL), dried over Na₂SO₄ and concentrated. The crude material was purified via silica gel chromatography (330 g SiO₂ column, dichloromethane:EtOAc 100:0->55:45) to afford ethyl-(S)-2-(5-bromo-2-chloro-4-(4,4-dimethylpiperidin-1-yl)pyridin-3-yl)-2-(tert-butoxy)acetate, 4.62 g (78%). LCMS (M+1)=461.1, 463.1. ¹H NMR (500 MHz, CDCl₃) δ 8.37 (s, 1H), 5.60 (br s, 1H), 4.28-4.11 (m, 2H), 3.91-3.63 (m, 1H), 3.33 (br s, 1H), 3.07 (br d, J=2.3 Hz, 1H), 2.58 (br d, J=1.5 Hz, 1H), 1.81-1.63 (m, 1H), 1.49-1.35 (m, 2H), 1.27 (br d, J=5.0 Hz, 1H), 1.25 (s, 9H), 1.02 (s, 6H).

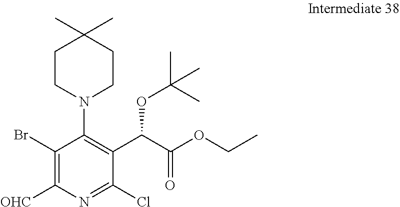 [SMILES: CCOC(=O)[C@@H](OC(C)(C)C)c1c(Cl)nc(C=O)c(Br)c1N1CCC(C)(C)CC1]

### Ethyl-(S)-2-(5-bromo-2-chloro-4-(4,4-dimethylpiperidin-1-yl)-6-formylpyridin-3-yl)-2-(tert-butoxy)acetate

To a dry 100 mL round bottom flask under argon was added ethyl-(S)-2-(5-bromo-2-chloro-4-(4,4-dimethylpiperidin-1-yl)pyridin-3-yl)-2-(tert-butoxy)acetate (1.05 g, 2.274 mmol) and THF (48 mL). The resulting solution was cooled to −25 C and then treated slowly (over 2 min) 2,2,6,6-tetramethylpiperidinylmagnesium chloride lithium chloride complex 1.0 M in THF/toluene (4.65 mL, 4.65 mmol). The reaction was stirred at −21° C. (+/−4 C) for 33 min, then recooled to −20° C. and treated with anhydrous DMF (1.00 mL, 12.91 mmol). The reaction was allowed to warm to −8° C. over 70 min, then recooled to −20° C. and quenched with aqueous saturated NH₄Cl. The crude reaction was diluted with ethyl acetate (150 mL), extracted with water (1×15 mL, brine (1×15 mL), dried over Na₂SO₄, and concentrated. The crude material was purified via silica gel chromatography (80 g SiO₂ column, dichloromethane:EtOAc 100:0->80:20) to afford ethyl (S)-2-(5-bromo-2-chloro-4-(4,4-dimethylpiperidin-1-yl)-6-formylpyridin-3-yl)-2-(tert-butoxy)acetate, 1.04 g (93%). LCMS (M+1)=489.1, 491.1.

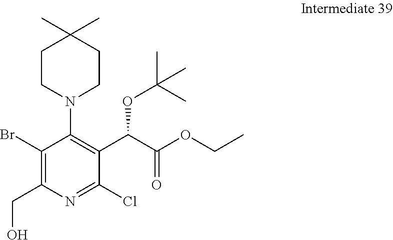 [SMILES: CCOC(=O)[C@@H](OC(C)(C)C)c1c(Cl)nc(CO)c(Br)c1N1CCC(C)(C)CC1]

### Ethyl (S)-2-(5-bromo-2-chloro-4-(4,4-dimethylpiperidin-1-yl)-6-(hydroxymethyl)pyridin-3-yl)-2-(tert-butoxy)acetate

To a dry reaction vial under nitrogen was added (S)-ethyl 2-(5-bromo-2-chloro-4-(4,4-dimethylpiperidin-1-yl)-6-formylpyridin-3-yl)-2-(tert-butoxy)acetate (20 mg, 0.041 mmol) and EtOH (1.5 mL). The resulting solution was treated with sodium borohydride (8.5 mg, 0.225 mmol), capped and stirred at room temp for 2 h. The reaction was diluted with ethyl acetate (75 mL), extracted with aq sat'd NH₄Cl (1 10 mL), water (1×10 mL), brine (1×10 mL), dried over Na₂SO₄ and concentrated to afford ethyl (S)-2-(5-bromo-2-chloro-4-(4,4-dimethylpiperidin-1-yl)-6-(hydroxymethyl)pyridin-3-yl)-2-(tert-butoxy)acetate (12 mg, 60%) was used “as is” without further purification. LCMS (M+1)=491.2, 493.1.

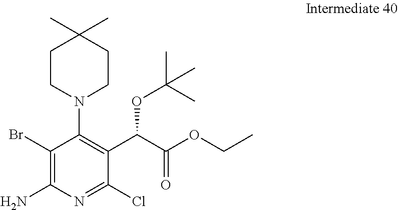 [SMILES: CCOC(=O)[C@@H](OC(C)(C)C)c1c(Cl)nc(N)c(Br)c1N1CCC(C)(C)CC1]

### Ethyl (S)-2-(6-amino-5-bromo-2-chloro-4-(4,4-dimethylpiperidin-1-yl)pyridin-3-yl)-2-(tert-butoxy)acetate

To a reaction vial under nitrogen was added (S)-ethyl 2-(5-bromo-2-chloro-4-(4,4-dimethylpiperidin-1-yl)-6-formylpyridin-3-yl)-2-(tert-butoxy)acetate (149 mg, 0.304 mmol) and acetonitrile (4 mL). The reaction was stirred at room temp, treated with a solution of oxone (380 mg, 0.618 mmol) in water (1.75 mL) and stirred at room temp for 20 h. The reaction was diluted with ethyl acetate (100 mL), extracted with water (1×8 mL) and evaporated to dryness. The crude residue was dissolved in toluene (4 mL) and treated with triethylamine (100.4 μL, 0.720 mmol), water (32.45 μL, 1.801 mmol), and diphenylphosphoryl azide (198 mg, 0.719 mmol). The reaction was flushed very briefly with nitrogen, capped and heated at 90° C. oil bath for 90 min. The reaction was diluted with ethyl acetate (100 mL), extracted with aq sat'd NaHCO₃ (1×10 mL), water (1×10 mL), brine (1×10 mL) dried over Na₂SO₄ and concentrated. The crude residue was purified via siliga gel chromotography (40 g SiO₂ column, hexane:EtOAc 100:0->70:30) to afford ethyl (S)-2-(6-amino-5-bromo-2-chloro-4-(4,4-dimethylpiperidin-1-yl)pyridin-3-yl)-2-(tert-butoxy)acetate, 100 mg (58%). LCMS (M+1)=476.1, 478.1.

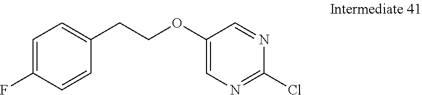 [SMILES: Fc1ccc(CCOc2cnc(Cl)nc2)cc1]

### 2-Chloro-5-(4-fluorophenethoxy)pyrimidine

To a 50 mL round bottom flask equipped with a stir bar was added 2-chloropyrimidin-5-ol (250 mg, 1.915 mmol), 2-(4-fluorophenyl)ethanol (268 mg, 1.915 mmol), triphenylphosphine (603 mg, 2.298 mmol) and THF (10 mL). To the stirred solution was added DIAD (0.447 mL, 2.298 mmol). The solution warmed to a mild exotherm, then cooled within 5 minutes. The solution was stirred at r.t. for 2 hrs. The reaction solution was concentrated in vacuo and the resulting oil was purified via silica gel chromatography (40 g column, 5-40% EtOAc:Hex) to afford the product 2-chloro-5-(4-fluorophenethoxy)pyrimidine (310 mg, 1.227 mmol, 64.1% yield) as a white solid. ¹H NMR (500 MHz, CDCl₃) δ 8.28 (s, 2H), 7.25 (dd, J=8.7, 5.4 Hz, 2H), 7.08-7.02 (m, 2H), 4.26 (t, J=6.7 Hz, 2H), 3.13 (t, J=6.7 Hz, 2H). LCMS (M+1)=252.9.

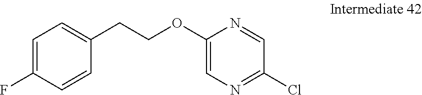 [SMILES: Fc1ccc(CCOc2cnc(Cl)cn2)cc1]

### 2-Chloro-5-(4-fluorophenethoxy)pyrazine

To a 50 mL round bottom flask equipped with a stir bar was added 5-chloropyrazin-2-ol (250 mg, 1.915 mmol), 2-(4-fluorophenyl)ethanol (268 mg, 1.915 mmol), triphenylphosphine (603 mg, 2.298 mmol) and THF (10 mL). To the stirred solution was added DIAD (0.447 mL, 2.298 mmol). The solution had a mild exotherm, then cooled within 5 minutes. The solution was stirred at RT for 2 hrs. The reaction solution was concentrated in vacuo and the resulting oil was purified via silica gel chromatography (40 g column, 5-40% EtOAc:Hex) to afford the product 2-chloro-5-(4-fluorophenethoxy)pyrazine (330 mg, 1.306 mmol, 68.2% yield) as a white solid. ¹H NMR (500 MHz, CDCl₃) δ 8.10 (d, J=1.3 Hz, 1H), 8.01 (d, J=1.3 Hz, 1H), 7.25 (dd, J=8.6, 5.4 Hz, 2H), 7.03 (t, J=8.7 Hz, 2H), 4.52 (t, J=6.9 Hz, 2H), 3.09 (t, J=6.9 Hz, 2H). LCMS (M+1)=253.0.

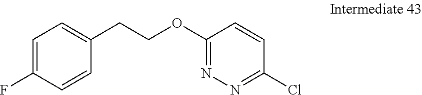 [SMILES: Fc1ccc(CCOc2ccc(Cl)nn2)cc1]

### 3-Chloro-6-(4-fluorophenethoxy)pyridazine

To a 50 mL round bottom flask equipped with a stir bar was added 6-chloropyridazin-3-ol (250 mg, 1.915 mmol), 2-(4-fluorophenyl)ethanol (268 mg, 1.915 mmol), triphenylphosphine (603 mg, 2.298 mmol) and THF (10 mL). To the stirred solution was added DIAD (0.447 mL, 2.298 mmol). The solution had a mild exotherm, then cooled within 5 minutes. The solution was stirred at RT for 2 hrs. The reaction solution was concentrated in vacuo and purified via silica gel chromatography (40 g column, 5-40% EtOAc:Hex) to afford the semi pure product. The material was further purified via reverse phase C18 chromatography (55 g column, 20-100% CH₃CN:Water with 0.1% TFA buffer). The desired fractions were isolated, diluted with sat. sodium bicarbonate solution (25 mL) and EtOAc. The organic layer was washed with brine, collected, dried over MgSO₄, and volatiles evaporated to afford the pure product 3-chloro-6-(4-fluorophenethoxy)pyridazine (450 mg, 1.781 mmol, 93% yield) as a white solid. 1H NMR (400 MHz, CDCl₃) δ 7.22 (dd, J=8.3, 5.4 Hz, 2H), 7.18 (d, J=9.8 Hz, 1H), 7.04-6.97 (m, 2H), 6.91 (d, J=9.5 Hz, 1H), 4.37-4.30 (m, 2H), 3.13-3.06 (m, 2H). LCMS (M+1)=253.0.

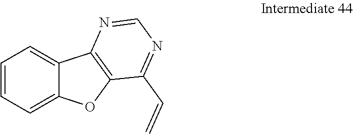

### 4-Vinylbenzofuro[3,2-d]pyrimidine

4-Chlorobenzofuro[3,2-d]pyrimidine (500 mg, 2.44 mmol), 6-methyl-2-vinyl-1,3,6,2-dioxazaborocane-4,8-dione (492 mg, 2.69 mmol), chloro(2-dicyclohexylphosphino-2′,6′-dimethoxy-1,1′-biphenyl)[2-(2′-amino-1,1′-biphenyl)]palladium(II) (176 mg, 0.244 mmol), potassium phosphate tribasic (3.89 g, 18.3 mmol) were combined under N₂ (g). 1,4-Dioxane (20 ml) and water (4.0 ml) was added under N₂ (g). The reaction was stirred at 80° C. for 1 hr. The reaction was concentrated, adsorbed onto celite and was purified on silica gel (Biotage, EtOAc/hexanes gradient, 0-100% over 10 CVs) to give the expected product 4-vinylbenzofuro[3,2-d]pyrimidine (254 mg, 1.30 mmol, 53.0% yield) LCMS (M+H)=196.80.

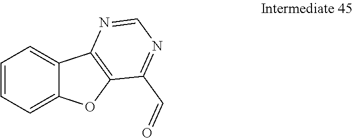 [SMILES: O=Cc1ncnc2c1oc1ccccc12]

### Benzofuro[3,2-d]pyrimidine-4-carbaldehyde

To a clear solution of 4-vinylbenzofuro[3,2-d]pyrimidine (254 mg, 1.30 mmol) in THF (5 ml) were sequentially added NMO (50% wt in H₂O) (607 mg, 2.59 mmol) and osmium tetroxide (4% in water) (198 μl, 0.032 mmol) at RT under nitrogen. The solution was vigorously stirred at 65° C. for 2 h. The reaction was cooled to room temperature and sodium periodate (1.1 g, 5.2 mmol) in H₂O (4 mL) was added [precipitate formed]. The mixture was stirred vigorously at RT under nitrogen for 1 hr. The mixture was diluted with EtOAc and washed with H₂O. The organic phase was concentrated, adsorbed onto celite and was purified on silica gel (Biotage, EtOAc/hexanes gradient) to give the expected product benzofuro[3,2-d]pyrimidine-4-carbaldehyde (84 mg, 0.424 mmol, 32.7% yield). LCMS (M+H)=198.93.

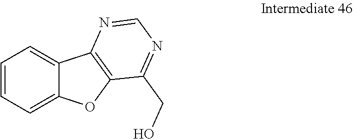 [SMILES: OCc1ncnc2c1oc1ccccc12]

### Benzofuro[3,2-d]pyrimidin-4-ylmethanol

The above crude benzofuro[3,2-d]pyrimidine-4-carbaldehyde (84 mg, 0.424 mmol) was taken up in MeOH and treated with 16 mg of NaBH₄ at rt. and stirred for 1 hr. The mixture was diluted with ethyl acetate and washed with sat NaHCO₃, and sat NaCl. The organic phase was dried over Na₂SO₄, filtered and concentrated, adsorbed onto celite and was purified on silica gel (Biotage, MeOH/DCM gradient, 0-100% over 10 CVs followed by 10 CVs of 10% MeOH/DCM) to give the expected product benzofuro[3,2-d]pyrimidin-4-ylmethanol (64 mg, 0.320 mmol, 24.69% yield). LCMS (M+H)=200.95.

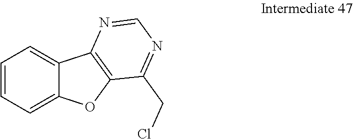 [SMILES: ClCc1ncnc2c1oc1ccccc12]

### 4-(Chloromethyl)benzofuro[3,2-d]pyrimidine

Ms-Cl (20 μL, 0.25 mmol) was added to a stirring solution of Hunig's base (44 μL, 0.25 mmol) and benzofuro[3,2-d]pyrimidin-4-ylmethanol (50 mg, 0.25 mmol) in DCM (2.5 ml) at 0° C., then stir at rt for 2 hrs. LCMS showed two peaks. The reaction was concentrated, adsorbed onto celite and was purified on silica gel (Biotage, EtOAc/hexanes gradient, 0-100% over 10 CVs). One of the products that was isolated was consistent with 4-(chloromethyl)benzofuro[3,2-d]pyrimidine (15 mg, 0.069 mmol, 27.5% yield). LCMS (M+H)=218.90.

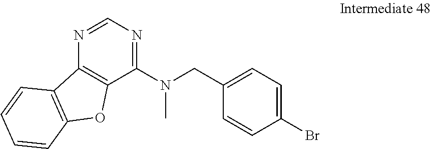 [SMILES: CN(Cc1ccc(Br)cc1)c1ncnc2c1oc1ccccc12]

### N-(4-Bromobenzyl)-N-methylbenzofuro[3,2-d]pyrimidin-4-amine

4-chlorobenzofuro[3,2-d]pyrimidine (102 mg, 0.500 mmol) was added to a stirring solution of 1-(4-bromophenyl)-N-methylmethanamine (100 mg, 0.500 mmol) in ACN (2.5 ml) at rt. The reaction was stirred at 90° C. overnight. The reaction was concentrated, adsorbed onto celite and was purified on silica gel (Biotage, EtOAc/hexanes gradient, 0-100% over 10 CVs) to give the expected product N-(4-bromobenzyl)-N-methylbenzofuro[3,2-d]pyrimidin-4-amine (174 mg, 0.473 mmol, 95% yield). LCMS (M+H)=367.90 and 369.90.

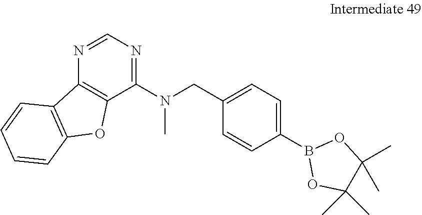 [SMILES: CN(Cc1ccc(B2OC(C)(C)C(C)(C)O2)cc1)c1ncnc2c1oc1ccccc12]

### N-Methyl-N-(4-(4,4,5,5-tetramethyl-1,3,2-dioxaborolan-2-yl)benzyl)benzofuro[3,2-d]pyrimidin-4-amine

N-(4-Bromobenzyl)-N-methylbenzofuro[3,2-d]pyrimidin-4-amine (174 mg, 0.473 mmol), 4,4,4′,4′,5,5,5′,5′-octamethyl-2,2′-bi(1,3,2-dioxaborolane) (180 mg, 0.709 mmol), PdCl₂dppf (35 mg, 0.047 mmol) and potassium acetate (139 mg, 1.42 mmol) were combined in dioxane (9.5 ml) in a sealed bottle. The mixture was degassed and heated at 85° C. for 2 hrs. The mixture was concentrated and the residue was purified by silica gel column (EtOAc/hexanes gradient 0-100% over 10 CVs) to give N-methyl-N-(4-(4,4,5,5-tetramethyl-1,3,2-dioxaborolan-2-yl)benzyl)benzofuro[3,2-d]pyrimidin-4-amine (quant yield). LCMS (M+H)=416.05.

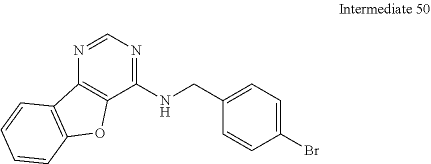 [SMILES: Brc1ccc(CNc2ncnc3c2oc2ccccc23)cc1]

### N-(4-Bromobenzyl)benzofuro[3,2-d]pyrimidin-4-amine

4-Chlorobenzofuro[3,2-d]pyrimidine (110 mg, 0.537 mmol) was added to a stirring solution of (4-bromophenyl)methanamine (100 mg, 0.537 mmol) in ACN (2.7 ml) at rt. The reaction was stirred at 90° C. overnight. The reaction was concentrated, adsorbed onto celite and was purified on silica gel (Biotage, EtOAc/hexanes gradient, 0-100% over 10 CVs) to afford N-(4-bromobenzyl)benzofuro[3,2-d]pyrimidin-4-amine (130 mg, 0.367 mmol, 68.3% yield). LCMS (M+H)=353.85 and 355.85.

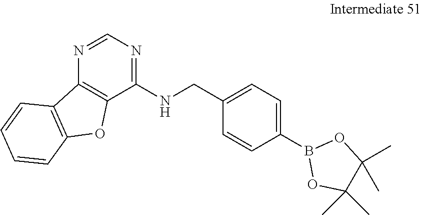 [SMILES: CC1(C)OB(c2ccc(CNc3ncnc4c3oc3ccccc34)cc2)OC1(C)C]

### N-(4-(4, 4, 5,5-Tetramethyl-1,3,2-dioxaborolan-2-yl)benzyl)benzofuro[3,2-d]pyrimidin-4-amine

N-(4-Bromobenzyl)benzofuro[3,2-d]pyrimidin-4-amine (65 mg, 0.18 mmol), 4,4,4′,4′,5,5,5′,5′-octamethyl-2,2′-bi(1,3,2-dioxaborolane) (70 mg, 0.28 mmol), PdCl2 (dppf) (13 mg, 0.018 mmol) and potassium acetate (54 mg, 0.55 mmol) were combined in dioxane (3.7 ml) in a sealed MW vial. The mixture was degassed and heated at 95° C. for 2 hrs. The mixture was concentrated and the residue was purified by silica gel column (EtOAc/hexanes gradient 0-100% over 10 CVs) to give N-(4-(4,4,5,5-tetramethyl-1,3,2-dioxaborolan-2-yl)benzyl)benzofuro[3,2-d]pyrimidin-4-amine (quant yield). LCMS (M+H)=402.05.

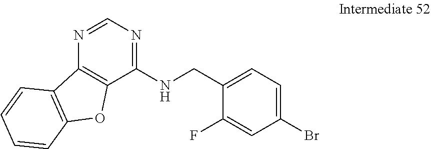 [SMILES: Fc1cc(Br)ccc1CNc1ncnc2c1oc1ccccc12]

### N-(4-Bromo-2-fluorobenzyl)benzofuro[3,2-d]pyrimidin-4-amine

A mixture of 4-chlorobenzofuro[3,2-d]pyrimidine (1.01 g, 4.94 mmol) and (4-bromo-2-fluorophenyl)methanamine (1.04 g, 5.10 mmol) in acetonitrile (50 mL) in a pressure bottle was treated with Hunig's base (1.6 mL, 9.16 mmol) and the resulting suspension stirred at RT for 10 min, then heated to 120° C. in an oil bath. After 5 hours the mixture went into solution and was then stirred for 16 hours overnight. A pale yellow solid had formed while at 120° C., then the reaction mixture was cooled to RT. The mixture was filtered and washed with CH₃CN, and was dried under vacuum to give 1.43 g (78%) of N-(4-bromo-2-fluorobenzyl)benzofuro[3,2-d]pyrimidin-4-amine as a pale yellow solid. LCMS (M+1)=371.6/373.6.

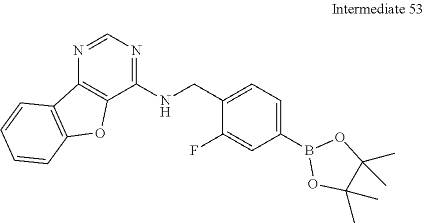 [SMILES: CC1(C)OB(c2ccc(CNc3ncnc4c3oc3ccccc34)c(F)c2)OC1(C)C]

### N-(2-Fluoro-4-(4,4,5,5-tetramethyl-1,3,2-dioxaborolan-2-yl)benzyl)benzofuro[3,2-d]pyrimidin-4-amine

In a pressure vessel equipped with a magnetic stirring bar was added N-(4-bromo-2-fluorobenzyl)benzofuro[3,2-d]pyrimidin-4-amine (1 g, 2.69 mmol), PdCl2(dppf)-CH2Cl2 adduct (0.219 g, 0.269 mmol), potassium acetate (0.791 g, 8.06 mmol) and 4,4,4′,4′,5,5,5′,5′-octamethyl-2,2′-bi(1,3,2-dioxaborolane) (1.023 g, 4.03 mmol). The solids were suspended in dioxane (15 mL). Argon was bubbled through the mixture for 5 minutes while sonicating. The vial was capped and heated to 80° C. within a preheated oil bath and allowed to continue for 16 hours. After 16 hours at 80° C., LC/MS showed the desired product as major. The reaction mixture was filtered, then concentrated down. The residue was purified by flash column chromatography to give 1.35 g (quant.) of N-(2-fluoro-4-(4,4,5,5-tetramethyl-1,3,2-dioxaborolan-2-yl)benzyl)benzofuro[3,2-d]pyrimidin-4-amine as an off-white sticky solid. LCMS (M+1, boronic acid fragment)=337.7.

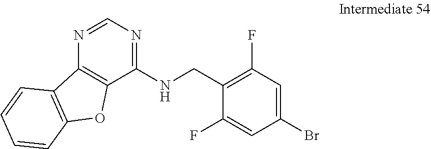 [SMILES: Fc1cc(Br)cc(F)c1CNc1ncnc2c1oc1ccccc12]

### N-(4-Bromo-2,6-difluorobenzyl)benzofuro[3,2-d]pyrimidin-4-amine

In a pressure vessel equipped with a magnetic stirring bar was added (4-bromo-2,6-difluorophenyl)methanamine.HCl (1 g, 3.87 mmol), and 4-chlorobenzofuro[3,2-d]pyrimidine (0.792 g, 3.87 mmol) in acetonitrile (20 mL). Hunig's base (2.70 mL, 15.47 mmol) was added and the mixture was heated to 80° C. in a preheated oil bath and allowed to stir for 48 hours overnight. Cool to RT and filter formed solids, wash with excess acetonitrile, filter and dry under vacuum to give 863 mg (57%) of N-(4-bromo-2,6-difluorobenzyl)benzofuro[3,2-d]pyrimidin-4-amine as a fluffy white solid. LCMS (M+1)=389.6 and 391.6.

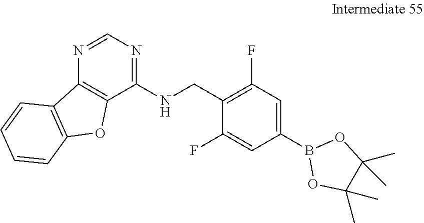 [SMILES: CC1(C)OB(c2cc(F)c(CNc3ncnc4c3oc3ccccc34)c(F)c2)OC1(C)C]

### N-(2,6-Bifluoro-4-(4, 4, 5,5-tetramethyl-1,3,2-dioxaborolan-2-yl)benzyl)benzofuro[3,2-d]pyrimidin-4-amine

In a pressure vessel equipped with a magnetic stirring bar was added recrystallized N-(4-bromo-2,6-difluorobenzyl)benzofuro[3,2-d]pyrimidin-4-amine (860 mg, 2.204 mmol), PdCl₂(dppf)*CH₂Cl₂ adduct (180 mg, 0.220 mmol), potassium acetate (649 mg, 6.61 mmol) and 4,4,4′,4′,5,5,5′,5′-octamethyl-2,2′-bi(1,3,2-dioxaborolane) (840 mg, 3.31 mmol). The solids were suspended in dioxane (15 mL). Argon was bubbled through the mixture for 5 minutes while sonicating. The flask was capped and heated to 80° C. within a preheated oil bath and allowed to continue for 16 hours. LC/MS showed the desired product (as the boronic acid) as major. The reaction mixture was filtered, then concentrated down. The residue was purified by flask column chromatography to give 1.28 g (quant.) of N-(2,6-difluoro-4-(4,4,5,5-tetramethyl-1,3,2-dioxaborolan-2-yl)benzyl)benzofuro[3,2-d]pyrimidin-4-amine as an off-white solid. LCMS (M+1, boronic acid fragment)=355.8.

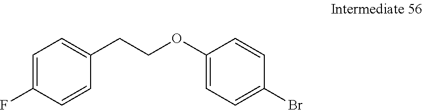 [SMILES: Fc1ccc(CCOc2ccc(Br)cc2)cc1]

### 1-Bromo-4-(4-fluorophenethoxy)benzene

To a stirred solution of 4-bromophenol (81.7 g, 472 mmol), 2-(4-fluorophenyl)ethanol (79 g, 567 mmol) and Ph₃P (149 g, 567 mmol) in THF (100 mL) cooled in an ice-water bath was added drop wise DEAD (93 ml, 590 mmol) over 20 min. Note: The reaction is exothermic and efficient cooling is highly recommended before initiating large scale reaction. After 1 h, cold bath was removed and stirred overnight (17 h) at rt. Then, the reaction mixture was concentrated, the resulting residue triturated with hexanes, filtered and the filter cake washed with 10% ether/hexanes (2-lit). The filtrate was concentrated and purified by flash chromatography (silica gel column 3″×11″) using 4-lit hexanes and 2-lit 2% EtOAc/Hex to afford 1-bromo-4-(4-fluorophenethoxy)benzene (142 g, 469 mmol, 99% yield) as colorless liquid (contaminated with −2.5% Ph₃P by ¹HNMR). ¹H NMR (500 MHz, CDCl₃) δ 7.41-7.36 (m, 2H), 7.28-7.22 (m, 2H), 7.05-6.99 (m, 2H), 6.82-6.76 (m, 2H), 4.14 (t, J=6.9 Hz, 2H), 3.08 (t, J=6.9 Hz, 2H).

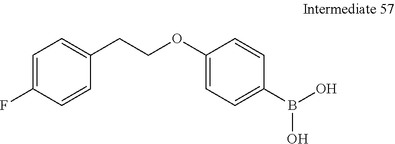 [SMILES: OB(O)c1ccc(OCCc2ccc(F)cc2)cc1]

### (4-(4-Fluorophenethoxy)phenyl)boronic acid

To a stirred solution of 1-bromo-4-(4-fluorophenethoxy)benzene (142 g, 469 mmol) in THF (1000 mL) was added 2M n-BuLi/cyclohexane (293 ml, 586 mmol) over 15 min at −78° C. After 1.5 h, triisopropyl borate (131 ml, 563 mmol) was added to the light pink reaction mixture over 5 min and stirred for 2 h at −78° C. Then, the reaction was quenched by careful addition of 3M HCl (375 mL), cold bath was replaced with water bath, stirred for 1 h, diluted with ether (500 mL), aq. layer separated and organic layer washed with water (2×200 mL). The combined aq. layers extracted with ether (200 mL) and combined ether layers washed with brine (100 mL), dried (MgSO₄), filtered and concentrated to 200 mL. To this was added 250 mL hexanes and concentrated to about 300 mL and allowed to stand at rt. The precipitated solid was triturated with hexanes and filtered to give white solid which was used in next step without purification. ¹H NMR (500 MHz, CDCl₃) δ 8.18-8.15 (m, 2H), 7.32-7.28 (m, 2H), 7.07-7.00 (m, 4H), 4.26 (t, J=6.9 Hz, 2H), 3.14 (t, J=6.9 Hz, 2H).

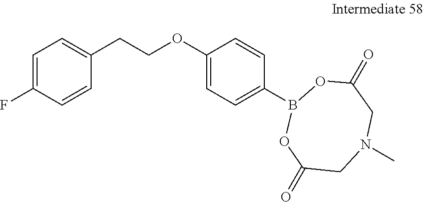 [SMILES: CN1CC(=O)OB(c2ccc(OCCc3ccc(F)cc3)cc2)OC(=O)C1]

### 2-(4-(4-Fluorophenethoxy)phenyl)-6-methyl-1, 3,6,2-dioxazaborocane-4,8-dione

A slurry of (4-(4-fluorophenethoxy)phenyl)boronic acid (122 g, 469 mmol) and 2,2′-(methylazanediyl)diacetic acid (76 g, 516 mmol) in anhydrous toluene (500 mL) and DMSO (200 mL) was refluxed for 4 h. Then, cooled, diluted with EtOAc (500 mL), washed with water (5×200 mL), brine (2×100 mL), dried (MgSO₄), filtered and concentrated to give light orange foam which was purified by flash chromatography using 5-40% acetone/CH₂Cl₂ (5% increment per 2-lit) to afford 2-(4-(4-fluorophenethoxy)phenyl)-6-methyl-1,3,6,2-dioxazaborocane-4,8-dione (131.38 g, 354 mmol, 75% yield) as white solid. ¹H NMR (500 MHz, CDCl₃) δ 7.43 (d, J=8.4 Hz, 2H), 7.28-7.24 (m, 2H), 7.04-6.99 (m, 2H), 6.92 (d, J=8.5 Hz, 2H), 4.17 (t, J=6.9 Hz, 2H), 4.00 (d, J=16.6 Hz, 2H), 3.76 (d, J=16.6 Hz, 2H), 3.08 (t, J=6.8 Hz, 2H), 2.54 (s, 3H). LCMS (M+H)=372.3.

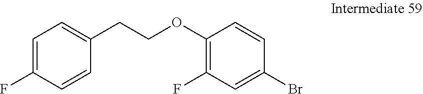 [SMILES: Fc1ccc(CCOc2ccc(Br)cc2F)cc1]

### 4-Bromo-2-fluoro-1-(4-fluorophenethoxy)benzene

To a 40 mL vial equipped with a stir bar was added 4-bromo-2-fluorophenol (954 mg, 5.00 mmol), 2-(4-fluorophenyl)ethanol (700 mg, 5.00 mmol), triphenylphosphine (2.62 g, 10 mmol) and THF (25 mL). To the stirred solution was added diisopropyl azodicarboxylate (“DIAD”, 1.9 mL, 10 mmol) upon which the resulting exotherm heated the solution to a mild reflux. The solution cooled to r.t. within 5 minutes, then was stirred at r.t. for 18 h. The solution was concentrated in vacuo and the resulting residue was diluted with a small amount of acetone, then was concentrated onto Celite in vacuo. The resulting powder was subjected to SiO₂ purification (80 g SiO₂ column, hexanes:EtOAc 100:0→90:10) to afford 4-bromo-2-fluoro-1-(4-fluorophenethoxy)benzene as a colorless oil, 1.31 g (84%). ¹H NMR (500 MHz, CDCl₃) δ 7.26-7.20 (m, 3H), 7.17-7.12 (m, 1H), 7.00 (t, J=8.7 Hz, 2H), 6.79 (t, J=8.7 Hz, 1H), 4.17 (t, J=6.9 Hz, 2H), 3.08 (t, J=6.9 Hz, 2H).

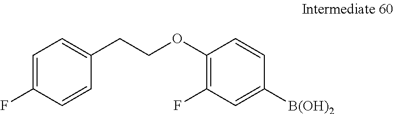 [SMILES: OB(O)c1ccc(OCCc2ccc(F)cc2)c(F)c1]

### (3-Fluoro-4-(4-fluorophenethoxy)phenyl)boronic acid

To a dry 25 mL Schlenk flask equipped with a stir bar was added a degassed (5 min N₂ bubbling) solution of 4-bromo-2-fluoro-1-(4-fluorophenethoxy)benzene (760 mg, 2.43 mmol) in THF (10 mL). The flask was cooled in a −78° C. bath. To the solution was added n-butyllithium (1.00 mL, 2.50 mmol). The solution was stirred for 5 minutes. A pale yellow color was noted. To the solution was added triisopropyl borate (0.845 mL, 3.64 mmol). The solution was stirred for 15 minutes. The cold bath was removed and the solution was allowed to warm to r.t. over 1 h. The turbid white solution was transferred to a 125 mL separatory funnel charged with aq HCl (1M, 10 mL). The mixture was extracted with Et₂O (30 mL). The organic phase was washed with brine (10 mL); dried over MgSO₄; filtered; then concentrated in vacuo to afford (3-fluoro-4-(4-fluorophenethoxy)phenyl)boronic acid as a golden oil.

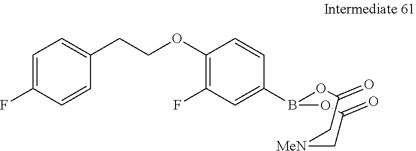 [SMILES: CN1CC(=O)OB(c2ccc(OCCc3ccc(F)cc3)c(F)c2)OC(=O)C1]

### 2-(3-Fluoro-4-(4-fluorophenethoxy)phenyl)-6-methyl-1, 3,6,2-dioxazaborocane-4,8-dione

To a 100 mL r.b. flask charged with the entirety of (3-fluoro-4-(4-fluorophenethoxy)phenyl)boronic acid prepared above was added N-methyliminodiacetic acid (500 mg, 3.40 mmol), DMSO (2 mL) and benzene (10 mL). The flask was fitted with a Dean-Stark trap pre-filled with benzene. The Dean-Stark trap was fitted with an air-cooled reflux condensor. The reaction flask was placed in a 125° C. oil bath with stirring. An active azeotrope was observed within 10 minutes. Heating and stirring was maintained for 30 min, then the mixture was cooled to r.t. The volatiles were removed in vacuo, then the reaction mixture was transferred to a 125 mL separatory funnel and was diluted with water (50 mL). The mixture was extracted with EtOAc (2×50 mL). The combined organics were washed with brine (15 mL); dried over MgSO₄; filtered; then concentrated in vacuo. The resulting residue was dissolved in a min. of acetone, then was concentrated onto Celite in vacuo. The resulting powder was subjected to SiO₂ purification (24 g SiO₂ column, hexanes:EtOAc 50:50-0:100, then 100% EtOAc) to 2-(3-fluoro-4-(4-fluorophenethoxy)phenyl)-6-methyl-1,3,6,2-dioxazaborocane-4,8-dione as a colorless solid foam, 777 mg (82% over two steps). ¹H NMR (500 MHz, acetone-d6) δ 7.40 (dd, J=8.7, 5.5 Hz, 2H), 7.28-7.19 (m, 2H), 7.13 (t, J=8.3 Hz, 1H), 7.10-7.03 (m, 2H), 4.33 (d, J=17.0 Hz, 2H), 4.29 (t, J=6.8 Hz, 2H), 4.14 (d, J=17.0 Hz, 2H), 3.12 (t, J=6.7 Hz, 2H), 2.76 (s, 3H).

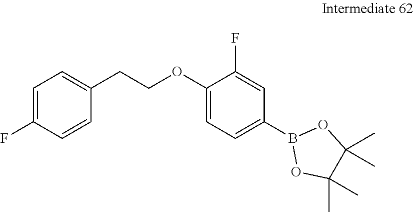 [SMILES: CC1(C)OB(c2ccc(OCCc3ccc(F)cc3)c(F)c2)OC1(C)C]

### 2-(3-Fluoro-4-(4-fluorophenethoxy)phenyl)-4,5,5-tetramethyl-1,3,2-dioxaborolane

To a dry 500 mL pressure bottle under nitrogen was added 4-bromo-2-fluoro-1-(4-fluorophenethoxy)benzene (6.3 g, 20.12 mmol), 4,4,4′,4′,5,5,5′,5′-octamethyl-2,2′-bi(1,3,2-dioxaborolane) (7.8 g, 30.7 mmol), potassium acetate (8 g, 82 mmol) and dioxane (175 mL). The reaction was flushed well with argon, treated with [1,1′-bis(diphenylphosphino)ferrocene]dichloropalladium(II) (800 mg, 1.093 mmol), capped and heated at 100° C. oil bath for 18 h. The crude reaction was diluted with ethyl acetate (450 mL), filtered through a pad of celite, extracted with water (1×150 mL), brine, dried over Na₂SO₄ and concentrated. The crude material was purified via silica gel chromatography (330 g SiO₂ column, hexane:dichloromethane 100:0->0:100) to afford 2-(3-fluoro-4-(4-fluorophenethoxy)phenyl)-4,4,5,5-tetramethyl-1,3,2-dioxaborolane, 3.95 g (55%). ¹H NMR (500 MHz, CDCl₃) δ 7.54-7.48 (m, 2H), 7.30-7.25 (m, 3H), 7.05-6.99 (m, 2H), 6.93 (t, J=8.0 Hz, 1H), 4.24 (t, J=6.9 Hz, 2H), 3.13 (t, J=6.9 Hz, 2H), 1.35 (s, 12H).

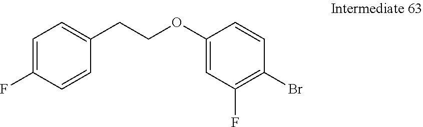 [SMILES: Fc1ccc(CCOc2ccc(Br)c(F)c2)cc1]

### 1-Bromo-2-fluoro-4-(4-fluorophenethoxy)benzene

To a 40 mL vial equipped with a stir bar was added 4-bromo-3-fluorophenol (954 mg, 5.00 mmol), 2-(4-fluorophenyl)ethanol (700 mg, 5.00 mmol), triphenylphosphine (1.57 g, 6.00 mmol) and THF (25 mL). To the stirred solution was added DIAD (1.17 mL, 6.00 mmol) upon which the resulting exotherm heated the solution to a mild reflux. The solution cooled to r.t. within 5 minutes. The solution was stirred at r.t. for 18 h. The solution was concentrated in vacuo and the residue was diluted with a small amount of acetone, then was concentrated onto Celite in vacuo. The resulting powder was subjected to SiO₂ purification (80 g SiO₂ column, hexanes:EtOAc 100:0→90:10) to afford 1-bromo-2-fluoro-4-(4-fluorophenethoxy)benzene as a yellow liquid, 1.28 g (82%). ¹H NMR (500 MHz, CDCl₃) δ 7.40 (dd, J=8.8, 8.0 Hz, 1H), 7.26-7.19 (m, 2H), 7.01 (t, J=8.7 Hz, 2H), 6.68 (dd, J=10.4, 2.8 Hz, 1H), 6.60 (ddd, J=8.8, 2.8, 1.0 Hz, 1H), 4.12 (t, J=6.8 Hz, 2H), 3.07 (t, J=6.8 Hz, 2H).

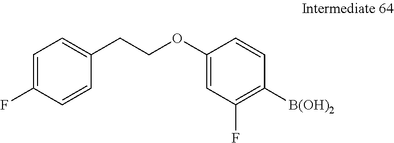 [SMILES: OB(O)c1ccc(OCCc2ccc(F)cc2)cc1F]

### (2-Fluoro-4-(4-fluorophenethoxy)phenyl)boronic acid

To a dry 25 mL Schlenk flask equipped with a stir bar was added a degassed (5 min N₂ bubbling) solution of 1-bromo-2-fluoro-4-(4-fluorophenethoxy)benzene (0.709 g, 2.27 mmol) in THF (10 mL). The flask was cooled in a −78° C. bath. To the solution was added n-butyllithium (1.00 mL, 2.50 mmol). The solution was stirred for 5 minutes. No color change was observed. To the solution was added triisopropyl borate (0.789 mL, 3.40 mmol). The solution was stirred for 15 minutes. The cold bath was removed and the solution was allowed to slowly warm to r.t. over 30 min., then the solution was stirred at r.t. for 18 h. The turbid white solution was transferred to a 125 mL separatory funnel charged with aq HCl (1M, 10 mL). The mixture was extracted with Et₂O (30 mL). The organic phase was washed with brine (10 mL); dried over MgSO₄; filtered; then concentrated in vacuo to afford (2-fluoro-4-(4-fluorophenethoxy)phenyl)boronic acid as a white solid.

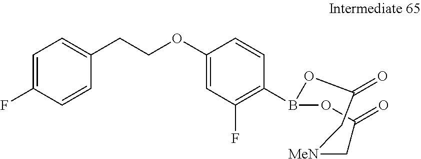 [SMILES: CN1CC(=O)OB(c2ccc(OCCc3ccc(F)cc3)cc2F)OC(=O)C1]

### 2-(2-Fluoro-4-(4-fluorophenethoxy)phenyl)-6-methyl-1, 3,6,2-dioxazaborocane-4,8-dione

To a 100 mL r.b. flask charged with the entirety of (2-fluoro-4-(4-fluorophenethoxy)phenyl)boronic acid prepared above was added N-methyliminodiacetic acid (500 mg, 3.40 mmol), DMSO (2 mL) and benzene (10 mL). The flask was fitted with a Dean-Stark trap pre-filled with benzene. The Dean-Stark trap was fitted with an air-cooled reflux condenser. The reaction flask was placed in a 125° C. oil bath with stirring. An active azeotrope was observed within 10 minutes. Heating and stirring was maintained for 30 min. The reaction mixture was cooled to r.t., then was transferred to a 125 mL separatory funnel and was diluted with water (50 mL). The mixture was extracted with EtOAc (2×50 mL). The combined organics were washed with brine (15 mL); dried over MgSO₄; filtered; then concentrated in vacuo. The resulting residue was dissolved in a min. of acetone, then was concentrated onto Celite in vacuo. The resulting powder was subjected to SiO₂ purification (80 g SiO₂ column, hexanes:EtOAc 100:0→90:10) to afford 2-(2-fluoro-4-(4-fluorophenethoxy)phenyl)-6-methyl-1,3,6,2-dioxazaborocane-4,8-dione as a colorless solid foam, 665 mg (75% over two steps). ¹H NMR (500 MHz, acetone-d6) δ 7.50-7.44 (m, 1H), 7.42-7.36 (m, 2H), 7.10-7.03 (m, 2H), 6.79 (dd, J=8.4, 2.2 Hz, 1H), 6.67 (dd, J=12.2, 2.3 Hz, 1H), 4.36 (dd, J=17.0, 1.1 Hz, 2H), 4.24 (t, J=6.7 Hz, 2H), 4.12 (d, J=17.0 Hz, 2H), 3.09 (t, J=6.7 Hz, 2H), 2.78 (s, 3H).

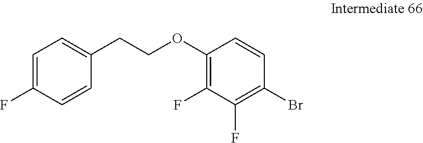 [SMILES: Fc1ccc(CCOc2ccc(Br)c(F)c2F)cc1]

To a 40 mL vial equipped with a stir bar was added 4-bromo-2,3-difluorophenol (1.04 g, 5.00 mmol), 2-(4-fluorophenyl)ethanol (700 mg, 5.00 mmol), triphenylphosphine (1.57 g, 6.00 mmol) and THF (25 mL). To the stirred solution was added DIAD (1.17 mL, 6.00 mmol) upon which the resulting exotherm heated the solution to a mild reflux. The solution cooled to r.t. within 5 minutes. The solution was stirred at r.t. for 18 h. The solution was concentrated in vacuo and the resulting oil was diluted with a min. of acetone, then concentrated onto Celite in vacuo. The resulting powder was subjected to SiO₂ purification (80 g SiO₂ column, hexanes:EtOAc 100:0→90:10) to afford 1-bromo-2,3-difluoro-4-(4-fluorophenethoxy)benzene as a colorless oil, 1.611 g (97%). ¹H NMR (500 MHz, CDCl₃) δ 7.26-7.21 (m, 2H), 7.18 (ddd, J=9.2, 7.0, 2.5 Hz, 1H), 7.04-6.97 (m, 2H), 6.62 (ddd, J=9.3, 7.4, 2.0 Hz, 1H), 4.19 (t, J=6.9 Hz, 2H), 3.09 (t, J=6.9 Hz, 2H).

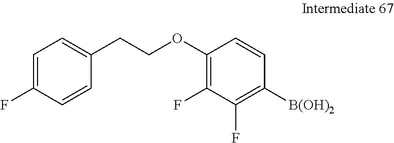 [SMILES: OB(O)c1ccc(OCCc2ccc(F)cc2)c(F)c1F]

To a dry 25 mL Schlenk flask equipped with a stir bar was added a degassed (5 min N₂ bubbling) solution of 1-bromo-2,3-difluoro-4-(4-fluorophenethoxy)benzene (750 mg, 2.27 mmol) in THF (10 mL). The flask was cooled in a −78° C. bath. To the solution was added n-butyllithium (1.00 mL, 2.50 mmol). The solution was stirred for 5 minutes. No color change was observed. To the solution was added triisopropyl borate (0.789 mL, 3.40 mmol). The solution was stirred for 15 minutes. The cold bath was removed and the solution was allowed to slowly warm to r.t. over 30 min, then the mixture was stirred at r.t. for 18 h. The turbid white solution was transferred to a 125 mL separatory funnel charged with aq. HCl (1M, 10 mL). The mixture was extracted with Et₂O (30 mL). The organic phase was washed with brine (10 mL); dried over MgSO₄; filtered; then concentrated in vacuo to afford (2,3-difluoro-4-(4-fluorophenethoxy)phenyl)boronic acid as a white solid.

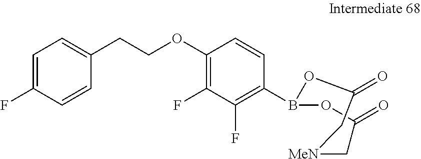 [SMILES: CN1CC(=O)OB(c2ccc(OCCc3ccc(F)cc3)c(F)c2F)OC(=O)C1]

To a 100 mL r.b. flask charged with the entirety of (2,3-difluoro-4-(4-fluorophenethoxy)phenyl)boronic acid prepared above was added N-methyliminodiacetic acid (500 mg, 3.40 mmol), DMSO (2 mL) and Benzene (10 mL). The flask was fitted with a Dean-Stark trap pre-filled with benzene. The Dean-Stark trap was fitted with an air-cooled reflux condenser. The reaction flask was placed in a 125° C. oil bath with stirring. An active azeotrope was observed within 10 minutes. Heating and stirring was maintained for 30 min. The reaction mixture was cooled to r.t., then was transferred to a 125 mL separatory funnel and was diluted with water (50 mL). The mixture was extracted with EtOAc (2×50 mL). The combined organics were washed with brine (15 mL); dried over MgSO₄; filtered; then concentrated in vacuo. The resulting residue was dissolved in a min. of acetone, then was concentrated onto Celite in vacuo. The resulting powder was subjected to SiO₂ purification (24 g SiO₂ column, hexanes:EtOAc 50:50→0:100, then 100% EtOAc) to afford 2-(2,3-difluoro-4-(4-fluorophenethoxy)phenyl)-6-methyl-1,3,6,2-dioxazaborocane-4,8-dione as a white crystalline solid, 732 mg (79% over two steps). ¹H NMR (500 MHz, acetone-d6) δ 2.88 (s, 3H) 3.13 (t, J=6.70 Hz, 2H) 4.16 (d, J=17.02 Hz, 2H) 4.33 (t, J=6.70 Hz, 2H) 4.37-4.43 (m, 2H) 6.99 (ddd, J=8.47, 7.05, 1.50 Hz, 1H) 7.04-7.11 (m, 2H) 7.24 (ddd, J=8.63, 6.82, 2.21 Hz, 1H) 7.37-7.43 (m, 1H).

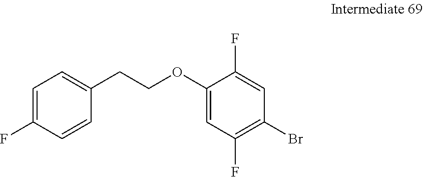 [SMILES: Fc1ccc(CCOc2cc(F)c(Br)cc2F)cc1]

### 1-Bromo-2,5-difluoro-4-(4-fluorophenethoxy)benzene

To a 40 mL vial equipped with a stir bar was added 4-bromo-2,5-difluorophenol (1.04 g, 5.00 mmol), 2-(4-fluorophenyl)ethanol (700 mg, 5.00 mmol), triphenylphosphine (1.57 g, 6.00 mmol) and THF (25 mL). To the stirred solution was added DIAD (1.17 mL, 6.00 mmol). The solution warmed to a mild reflux, then cooled within 5 minutes. The solution was stirred at r.t. for 18 h. The reaction solution was concentrated in vacuo and the resulting oil was diluted with a min. of acetone, then concentrated onto Celite in vacuo. The resulting powder was subjected to SiO₂ purification (80 g column, hexanes:EtOAc 100:0→90:10) to afford 1-bromo-2,5-difluoro-4-(4-fluorophenethoxy)benzene as a colorless oil (1.562 g, 94%). ¹H NMR (500 MHz, CDCl₃) δ 7.28-7.21 (m, 3H), 7.04-6.96 (m, 2H), 6.73 (dd, J=9.5, 7.2 Hz, 1H), 4.16 (t, J=6.9 Hz, 2H), 3.09 (t, J=6.9 Hz, 2H).

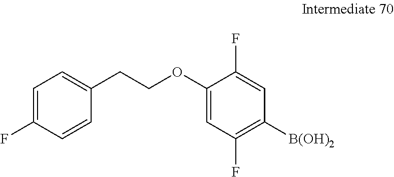 [SMILES: OB(O)c1cc(F)c(OCCc2ccc(F)cc2)cc1F]

### (2,5-Difluoro-4-(4-fluorophenethoxy)phenyl)boronic acid

To a dry 25 mL Schlenk flask equipped with a stir bar was added a degassed (5 min. N₂ bubbling) solution of 1-bromo-2,5-difluoro-4-(4-fluorophenethoxy)benzene (0.750 g, 2.27 mmol) in THF (10 mL). The flask was cooled in a −78° C. bath. To the solution was added n-butyllithium (1.00 mL, 2.50 mmol). The solution was stirred for 5 minutes. To the solution was added triisopropyl borate (0.789 mL, 3.40 mmol). The solution was stirred for 15 minutes. The cold bath was removed and the solution was allowed to slowly warm to r.t. After 18 h, the turbid, pale yellow reaction solution was transferred to a 125 mL separatory funnel charged with aq. HCl (1M, 10 mL). The mixture was extracted with Et₂O (30 mL). The organic phase was washed with brine (10 mL), then dried over MgSO₄, then filtered, then concentrated in vacuo to afford crude (2,5-difluoro-4-(4-fluorophenethoxy)phenyl)boronic acid as a pale yellow oil residue.

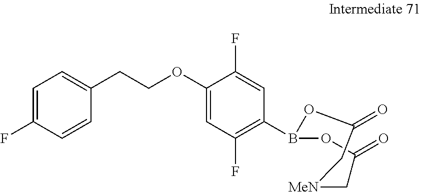 [SMILES: CN1CC(=O)OB(c2cc(F)c(OCCc3ccc(F)cc3)cc2F)OC(=O)C1]

### 2-(2,5-Difluoro-4-(4-fluorophenethoxy)phenyl)-6-methyl-1, 3,6,2-dioxazaborocane-4,8-dione

To a 100 mL r.b. flask charged with (2,5-difluoro-4-(4-fluorophenethoxy)phenyl)boronic acid prepared above was added N-methyliminodiacetic acid (500 mg, 3.40 mmol), DMSO (2 mL) and Benzene (10 mL). The flask was fitted with a Dean-Stark trap pre-filled with benzene. The Dean-Stark trap was fitted with an air-cooled reflux condenser. The reaction flask was placed in a 125° C. oil bath with stirring. An active azeotrope was observed within 10 minutes. Heating and stirring was maintained for 30 min. The reaction mixture was cooled to r.t., then was transferred to a 125 mL separatory funnel and was diluted with water (50 mL). The mixture was extracted with EtOAc (2×50 mL). The combined organics were washed with brine (15 mL); dried over MgSO₄; filtered; then concentrated in vacuo. The resulting residue was dissolved in a min. of acetone, then was concentrated onto Celite in vacuo. The resulting powder was subjected to SiO₂ purification (24 g SiO₂ column, hexanes:EtOAc 50:50→0:100, then 100% EtOAc) to afford 2-(2,5-difluoro-4-(4-fluorophenethoxy)phenyl)-6-methyl-1,3,6,2-dioxazaborocane-4,8-dione as a white solid (681 mg, 74% yield over two steps). ¹H NMR (500 MHz, acetone-d6) δ 7.43-7.37 (m, 2H), 7.22 (dd, J=11.7, 6.0 Hz, 1H), 7.11-7.03 (m, 2H), 6.93-6.92 (m, 1H), 6.93 (dd, J=11.0, 6.6 Hz, 1H), 4.39 (dd, J=17.0, 1.1 Hz, 2H), 4.31 (t, J=6.7 Hz, 2H), 4.14 (d, J=17.3 Hz, 2H), 3.13 (t, J=6.7 Hz, 2H), 2.89 (s, 3H), 2.80 (d, J=17.0 Hz, 2H).

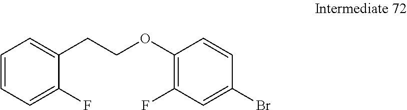 [SMILES: Fc1ccccc1CCOc1ccc(Br)cc1F]

### 4-Bromo-2-fluoro-1-(2-fluorophenethoxy)benzene

To a 100 mL r.b. flask equipped with a stir bar was added 2-(2-fluorophenyl)ethanol (2.00 g, 14.3 mmol), 4-bromo-2-fluorophenol (3.27 g, 17.1 mmol), triphenylphosphine (5.24 g, 20.0 mmol), and THF (28 mL). To the stirred solution was added dropwise DIAD (3.88 mL, 20.0 mmol). After stirring 1 h, the reaction solution was diluted with Et₂O and then washed with 10% K₂CO₃ in water. The organic phase was washed brine, then dried over MgSO₄, then filtered, then concentrated in vacuo. The resulting residue was subjected to SiO₂ purification (hexane:EtOAc 100:0→50:50) to afford 4-bromo-2-fluoro-1-(2-fluorophenethoxy)benzene as a colorless oil (4.16 g, 93%). ¹H NMR (500 MHz, CDCl₃) δ 7.33 (td, J=7.5, 1.8 Hz, 1H), 7.29-7.23 (m, 2H), 7.18 (dt, J=8.4, 2.1 Hz, 1H), 7.12 (td, J=7.5, 1.1 Hz, 1H), 7.07 (ddd, J=9.9, 8.4, 1.1 Hz, 1H), 6.85 (t, J=8.7 Hz, 1H), 4.24 (t, J=7.1 Hz, 2H), 3.19 (t, J=7.1 Hz, 2H).

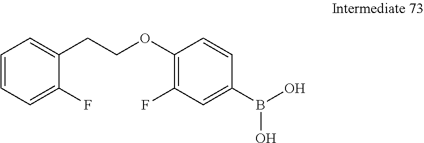 [SMILES: OB(O)c1ccc(OCCc2ccccc2F)c(F)c1]

### (3-Fluoro-4-(2-fluorophenethoxy)phenyl)boronic acid

To a dry 50 mL Schlenk flask equipped with a stir bar was added a degassed (5 min N₂ bubbling) solution of 4-bromo-2-fluoro-1-(2-fluorophenethoxy)benzene (1.57 g, 5.00 mmol) in THF (20 mL). The flask was cooled in a −78° C. bath. To the solution was added n-butyllithium (2.5M in hexane, 2.20 mL, 5.50 mmol). The solution was stirred for 5 minutes. To the solution was added triisopropyl borate (1.74 mL, 7.50 mmol). The solution was stirred for 15 minutes. The cold bath was removed and the solution was allowed to slowly warm to r.t. with stirring for 7 d (reaction time not optimized). The reaction mixture was transferred to a 125 mL separatory funnel charged with aq HCl (1M, 20 mL). The mixture was extracted with Et₂O (60 mL). The organic phase was washed with brine (20 mL), then dried over MgSO₄, then filtered, then concentrated in vacuo to afford (3-fluoro-4-(2-fluorophenethoxy)phenyl)boronic acid as an off-white solid (1.3463 g, 97%). ¹H NMR (500 MHz, methanol-d4) δ 7.49 (br d, J=8.0 Hz, 1H), 7.43 (br d, J=12.5 Hz, 1H), 7.38 (td, J=7.6, 1.6 Hz, 1H), 7.34 (br d, J=12.3 Hz, 1H), 7.27 (tdd, J=7.8, 5.4, 1.9 Hz, 1H), 7.13 (td, J=7.5, 1.2 Hz, 1H), 7.10-7.05 (m, 1H), 4.28 (br t, J=6.6 Hz, 2H), 3.16 (t, J=6.8 Hz, 2H)

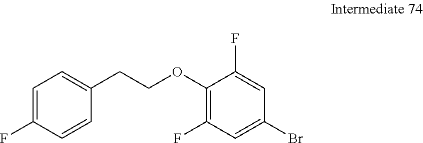 [SMILES: Fc1ccc(CCOc2c(F)cc(Br)cc2F)cc1]

### 5-Bromo-1,3-difluoro-2-(4-fluorophenethoxy)benzene

To a 40 mL vial equipped with a stir bar was added 4-bromo-2,6-difluorophenol (1.04 g, 5.00 mmol), 2-(4-fluorophenyl)ethanol (700 mg, 5.00 mmol), triphenylphosphine (1.57 g, 6.00 mmol) and THF (25 mL). To the stirred solution was added DIAD (1.17 mL, 6.00 mmol). The solution warmed to a mild reflux, then cooled within 5 minutes. The solution was stirred at r.t. for 18 h. The reaction solution was concentrated in vacuo. The resulting residue was dissolved in a min. of acetone, then was concentrated onto Celite in vacuo. The resulting powder was subjected to SiO₂ purification (80 g column, hexanes:EtOAc 100:0→80:20) to afford 5-bromo-1,3-difluoro-2-(4-fluorophenethoxy)benzene (1.552 g, 94%). ¹H NMR (500 MHz, CDCl₃) δ 7.26-7.21 (m, 2H), 7.11-7.04 (m, 2H), 7.03-6.97 (m, 2H), 4.30 (t, J=6.9 Hz, 2H), 3.06 (t, J=6.9 Hz, 2H).

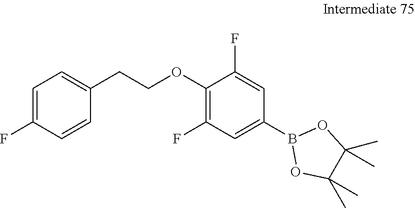 [SMILES: CC1(C)OB(c2cc(F)c(OCCc3ccc(F)cc3)c(F)c2)OC1(C)C]

### 2-(3,5-Difluoro-4-(4-fluorophenethoxy)phenyl)-4,4,5,5-tetramethyl-1,3,2-dioxaborolane

To a dry 10 mL Schlenk flask equipped with a stir bar was added potassium acetate (445 mg, 4.53 mmol), Pd(dppf)Cl:DCM complex (123 mg, 0.151 mmol), and bis(pinacolato)diboron (575 mg, 2.27 mmol). The flask was sealed with a rubber septum, then placed under N₂ atmosphere. To the flask was added a degassed (N₂ bubbling for 5 min) solution of 5-bromo-1,3-difluoro-2-(4-fluorophenethoxy)benzene (500 mg, 1.51 mmol) in dioxane (10 mL). The flask was placed in a 80° C. oil bath with stirring for Id. The reaction mixture was cooled to r.t., then was transferred to a 125 mL separatory funnel. The mixture was diluted with aq. NaOH (1M, 25 mL), then was extracted with Et₂O (50 mL). The organic phase was washed with brine (25 mL), then dried over MgSO₄, then filtered, then concentrated in vacuo. The resulting residue was dissolved in a min. of acetone, then was concentrated onto Celite in vacuo. The resulting powder was subjected to SiO₂ chromatogrpahy (40 g column, hexanes:EtOAc 100:04→90:10) to afford a mixture of 2-(3,5-difluoro-4-(4-fluorophenethoxy)phenyl)-4,4,5,5-tetramethyl-1,3,2-dioxaborolane and (3,5-difluoro-4-(4-fluorophenethoxy)phenyl)boronic acid as a solid (115 mg, 20%). ¹H NMR (500 MHz, CDCl₃, spectrum for pinacol ester reported) δ 7.32 (d, J=9.0 Hz, 2H), 7.28-7.24 (m, 2H), 7.08-6.98 (m, 2H), 4.38 (t, J=7.0 Hz, 2H), 3.08 (t, J=5.0 Hz, 2H), 1.35 (s, 12H).

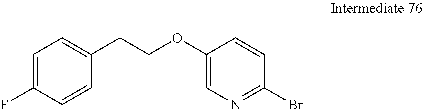 [SMILES: Fc1ccc(CCOc2ccc(Br)nc2)cc1]

### 2-Bromo-5-(4-fluorophenethoxy)pyridine

To a 40 mL vial equipped with a stir bar was added 1-(2-bromoethyl)-4-fluorobenzene (2.334 g, 11.49 mmol) and 6-bromopyridin-3-ol (1.00 g, 5.75 mmol), then DMSO (30 ml) and potassium carbonate (1.589 g, 11.49 mmol). The vial was vented to a N₂ stream (bubbler), then placed in a 50° C. oil bath with stirring for 18 h. (t=0). To the reaction solution was added 1-(2-bromoethyl)-4-fluorobenzene (2.334 g, 11.49 mmol). The mixture was stirred at 50° C. for 3 h. To the reaction mixture was added potassium carbonate (1.589 g, 11.49 mmol). The reaction mixture was stirred for 18 h. The reaction mixture was cooled to r.t., then was transferred to a 500 mL separatory funnel and was diluted with water (150 mL). The mixture was extracted with Et₂O (100 mL). The organic phase was washed with brine (50 mL), then dried over MgSO₄, filtered, then concentrated in vacuo. The resulting residue was dissolved in a min. of acetone and then was concentrated onto Celite in vacuo. The resulting powder was subjected to SiO₂ purification (80 g SiO₂, hexanes:EtOAc 100:0→80:20) to afford a colorless oil that crystallized upon standing to afford 2-bromo-5-(4-fluorophenethoxy)pyridine (1.5337 g, 90%) as a colorless, crystalline solid. ¹H NMR (500 MHz, CDCl₃) δ 8.04 (d, J=3.0 Hz, 1H), 7.36 (dd, J=8.7, 0.5 Hz, 1H), 7.26-7.21 (m, 2H), 7.08 (dd, J=8.8, 3.2 Hz, 1H), 7.05-6.99 (m, 2H), 4.18 (t, J=6.8 Hz, 2H), 3.08 (t, J=6.8 Hz, 2H).

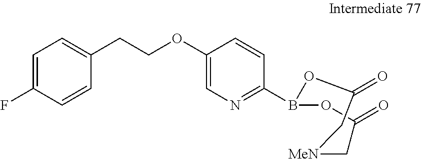 [SMILES: CN1CC(=O)OB(c2ccc(OCCc3ccc(F)cc3)cn2)OC(=O)C1]

### 2-(5-(4-Fluorophenethoxy)pyridin-2-yl)-6-methyl-1, 3,6,2-dioxazaborocane-4,8-dione

To a dry 50 mL Schlenk flask equipped with a large stir bar was added 2-bromo-5-(4-fluorophenethoxy)pyridine (6.103 g, 20.61 mmol). The flask was placed under nitrogen atmosphere. To the flask was added THF (36 ml) and triisopropyl borate (4.83 ml, 20.8 mmol). The flask was sealed with a septum, then the solution was sparged with N₂ for 5 minutes. The flask was cooled in a −78° C. bath. To the solution was added dropwise n-butyllithium in hexanes (8.33 ml, 20.8 mmol) at a rate necessary to avoid build-up of any localized dark discoloration. The rate was approximately 0.25-0.50 mL/min. At the completion of the addition the solution slowly began to turn a deep amber color. The mixture was stirred for 1 h up which the solution was observed to be a deep amber color. The bath was removed and the solution was allowed to warm to r.t. with stirring for 3 h. Separately, a distillation apparatus was assembled as follows: a 3-neck 250 mL flask equipped with a large stir bar was charged with methyliminodiacetic acid (6.06 g, 41.2 mmol) and DMSO (36.2 ml); the center neck was fitted with a pressure-equalizing addition funnel vented to positive N₂ pressure; another neck was fitted with a rubber septum through which a thermocouple was inserted to monitor internal temperature; and the final neck was fitted with a short-path distillation apparatus collecting into a 250 mL r.b. flask and vented to a bubbler. The reaction solution containing the boronate was transferred to the addition funnel. The 3-neck flask was heated with an oil bath (160° C.). Once the internal temperature had reached 115-120° C., the boronate solution was added dropwise at a rate necessary to maintain an internal temp of 115-120° C. The addition took approximately 20 min. The receiver flask containing the THF was exchanged for an empty 200 mL r.b. flask. The bubbler line connected to the vacuum arm of the distillation apparatus was exchanged for a tube running vacuum. The N₂ source was closed. The system was placed under vacuum, slowly ramping to 30 Torr upon which the DMSO distilled. The distillation was maintained at 30 Torr until only trace DMSO remained. The resulting residue, a solid, was dissolved in MeCN upon which only a white powder did not dissolve. The mixture was concentrated onto Celite in vacuo. The resulting powder was subjected to SiO₂ purification (120 g SiO₂ column, EtOAc:MeCN 100:0→0:100) on the Biotage to afford 2-(5-(4-fluorophenethoxy)pyridin-2-yl)-6-methyl-1,3,6,2-dioxazaborocane-4,8-dione (2.067 g, 27.0%) as a colorless solid foam. ¹H NMR (500 MHz, acetone-d6) δ 8.40-8.36 (m, 1H), 7.57-7.52 (m, 1H), 7.44-7.35 (m, 2H), 7.29 (dd, J=8.4, 2.8 Hz, 1H), 7.12-7.02 (m, 2H), 4.31 (d, J=16.6 Hz, 2H), 4.30 (t, J=6.7 Hz, 2H), 4.14 (d, J=16.6 Hz, 2H), 3.11 (t, J=6.7 Hz, 2H), 2.75 (s, 3H).

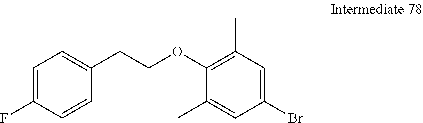 [SMILES: Cc1cc(Br)cc(C)c1OCCc1ccc(F)cc1]

### 5-Bromo-2-(4-fluorophenethoxy)-1,3-dimethylbenzene

To a solution of 4-bromo-2,6-dimethylphenol (1.0 g, 4.99 mmol), 2-(4-fluorophenyl)ethanol (0.70 g, 4.99 mmol), and triphenylphosphine (2.6 g, 9.99 mmol) in THF (25 mL) was added DIAD (1.94 mL, 9.99 mmol). The solution warmed to a mild reflux, then cooled to r.t. within 5 min. After stirring 18 h, the reaction solution was concentrated in vacuo. The resulting residue was purified by silica gel flash column chromatography (0-10% EtOAc/hexane) to provide the product as a colorless oil (1.50 g, 93%). ¹H NMR (500 MHz, CDCl₃) δ 7.28-7.22 (m, 2H), 7.12 (s, 2H), 7.04-6.98 (m, 2H), 3.91 (t, J=6.7 Hz, 2H), 3.06 (t, J=6.7 Hz, 2H), 2.13 (s, 6H).

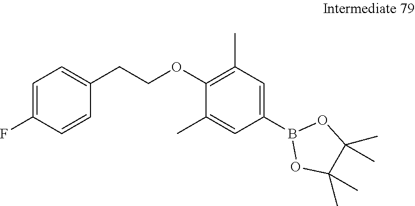 [SMILES: Cc1cc(B2OC(C)(C)C(C)(C)O2)cc(C)c1OCCc1ccc(F)cc1]

### 2-(4-(4-Fluorophenethoxy)-3,5-dimethylphenyl)-4,4,5,5-tetramethyl-1,3,2-dioxaborolane

A solution of potassium acetate (455 mg, 4.64 mmol), Pd(dppf)Cl₂ DCM complex (126 mg, 0.155 mmol), 4,4,4′,4′,5,5,5′,5′-octamethyl-2,2′-bi(1,3,2-dioxaborolane) (589 mg, 2.321 mmol), 5-bromo-2-(4-fluorophenethoxy)-1,3-dimethylbenzene (500 mg, 1.547 mmol) in dioxane (10 mL) was heated at 80° C. for 18 h. The reaction mixture was diluted with 1 M NaOH and extracted with ether. The organic phase was washed with brine (25 mL), dried (MgSO₄), and concentrated in vacuo. The resulting residue was purified by silica gel column chromatography (0-10% EtOAc/hexane) to give a white solid (0.44 g, 77%). ¹H NMR (500 MHz, CDCl₃) δ 7.48 (s, 2H), 7.31-7.26 (m, 2H), 7.02 (t, J=8.7 Hz, 2H), 3.97 (t, J=6.9 Hz, 2H), 3.09 (t, J=6.8 Hz, 2H), 2.19 (s, 6H), 1.35 (s, 12H).

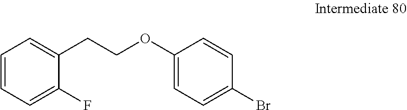 [SMILES: Fc1ccccc1CCOc1ccc(Br)cc1]

### 1-(2-(4-Bromophenoxy)ethyl)-2-fluorobenzene

To a solution of 2-(2-fluorophenyl)ethanol (2.85 g, 20.3 mmol, 1 equiv), 4-bromophenol (4.22 g, 24.4 mmol, 1.2 equiv), and triphenylphosphine (7.5 g, 28.5 mmol, 1.4 equiv) in THF (41 mL) was added DIAD (5.5 mL, 28.5 mmol, 1.4 equiv) slowly with a syringe. After stirring 1 h, the reaction was diluted with ether and washed with 10% aqueous potassium carbonate and brine. The ether layer was dried (MgSO₄) and concentrated in vacuo. The crude product was purified by silica gel flash chromatography to provide the product (4.80 g, 80%) as a colorless oil. ¹H NMR (500 MHz, CDCl₃) δ 7.40-7.36 (m, 2H), 7.33-7.30 (m, 1H), 7.27-7.23 (m, 1H), 7.11 (br td, J=7.5, 1.1 Hz, 1H), 7.07 (br ddd, J=9.9, 8.4, 1.0 Hz, 1H), 6.82-6.77 (m, 2H), 4.17 (t, J=7.0 Hz, 2H), 3.15 (t, J=6.9 Hz, 2H).

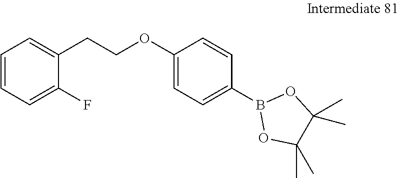 [SMILES: CC1(C)OB(c2ccc(OCCc3ccccc3F)cc2)OC1(C)C]

### 2-(4-(2-Fluorophenethoxy)phenyl)-4,4,5,5-tetramethyl-1,3,2-dioxaborolane

A solution of 1-(2-(4-bromophenoxy)ethyl)-3-fluorobenzene (0.82 g, 2.78 mmol, 1 equiv), 4,4,4′,4′,5,5,5′,5′-octamethyl-2,2′-bi(1,3,2-dioxaborolane) (1.06 g, 4.17 mmol, 1.5 equiv), Pd(dppf)Cl₂ (0.20 g, 0.278 mmol, 0.1 equiv), and KOAc (0.82 g, 8.33 mmol, 3 equiv) in dioxane (14 mL) was heated at 80° C. for 18 h. After cooling to ambient temperature, the reaction was diluted with EtOAc and washed with brine. The organic layer was dried (Na₂SO₄) and concentrated in vacuo. The crude product was purified by silica gel flash chromatography (0-60% EtOAc in hexane) to provide the product (0.63 g, 66%) as a waxy yellow solid. ¹H NMR (500 MHz, CDCl₃) δ 7.76 (d, J=8.5 Hz, 2H), 7.33-7.29 (m, 1H), 7.08 (d, J=7.6 Hz, 1H), 7.02 (dd, J=9.9, 2.0 Hz, 1H), 6.95 (td, J=8.6, 2.5 Hz, 1H), 6.91 (d, J=8.7 Hz, 2H), 4.22 (t, J=6.9 Hz, 2H), 3.12 (t, J=6.9 Hz, 2H), 1.36 (s, 12H). LCMS (M+1): 343.30.

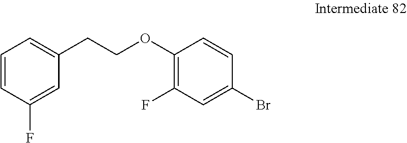 [SMILES: Fc1cccc(CCOc2ccc(Br)cc2F)c1]

### 4-Bromo-2-fluoro-1-(3-fluorophenethoxy)benzene

To a solution of 2-(3-fluorophenyl)ethanol (2.5 g, 17.8 mmol, 1 equiv), 4-bromo-2-fluorophenol (4.09 g, 21.4 mmol, 1.2 equiv), and triphenylphosphine (6.55 g, 25.0 mmol, 1.4 equiv) in THF (36 mL) was added DIAD (4.9 mL, 25.0 mmol, 1.4 equiv) slowly with a syringe. After stirring 1 h, the reaction was diluted with ether and washed with 10% aqueous potassium carbonate and brine. The ether layer was dried (MgSO₄) and concentrated in vacuo. The crude product was purified by silica gel flash chromatography to provide the product (5.1 g, 92%) as a colorless oil. ¹H NMR (500 MHz, CDCl₃) δ 7.34-7.28 (m, 1H), 7.26 (dd, J=10.6, 2.4 Hz, 1H), 7.20-7.15 (m, 1H), 7.08 (d, J=7.6 Hz, 1H), 7.03 (dt, J=9.9, 1.9 Hz, 1H), 6.99-6.93 (m, 1H), 6.83 (t, J=8.7 Hz, 1H), 4.23 (t, J=6.9 Hz, 2H), 3.14 (t, J=6.9 Hz, 2H).

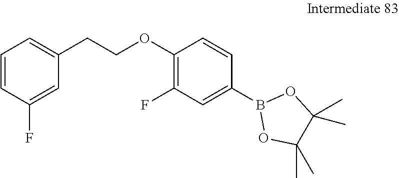 [SMILES: CC1(C)OB(c2ccc(OCCc3cccc(F)c3)c(F)c2)OC1(C)C]

### 2-(3-Fluoro-4-(3-fluorophenethoxy)phenyl)-5,4,5,5-tetramethyl-1,3,2-dioxaborolane

A solution of 4-bromo-2-fluoro-1-(3-fluorophenethoxy)benzene (1.28 g, 4.09 mmol, 1 equiv), 4,4,4′,4′,5,5,5′,5′-octamethyl-2,2′-bi(1,3,2-dioxaborolane) (1.56 g, 6.13 mmol, 1.5 equiv), Pd(dppf)Cl₂ (0.30 g, 0.409 mmol, 0.1 equiv), and KOAc (1.20 g, 12.3 mmol, 3 equiv) in dioxane (14 mL) was heated at 80° C. for 18 h. After cooling to ambient temperature, the reaction was diluted with EtOAc and washed with brine. The organic layer was dried (Na₂SO₄) and concentrated in vacuo. The crude product was purified by silica gel flash chromatography (0-60% EtOAc in hexane) to provide the product (0.59 g, 40%) as a waxy yellow solid. ¹H NMR (500 MHz, CDCl₃) δ 7.58-7.42 (m, 2H), 7.33-7.29 (m, 1H), 7.09 (d, J=7.6 Hz, 1H), 7.03 (dt, J=9.8, 2.0 Hz, 1H), 6.98-6.91 (m, 2H), 4.27 (t, J=6.9 Hz, 2H), 3.16 (t, J=6.9 Hz, 2H), 1.35 (s, 12H). LCMS (M+1): 361.10.

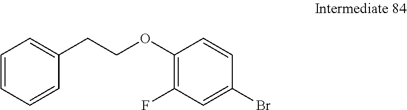 [SMILES: Fc1cc(Br)ccc1OCCc1ccccc1]

### 4-Bromo-2-fluoro-1-phenethoxybenzene

To a solution of 2-phenylethanol (3.0 g, 24.6 mmol, 1 equiv), 4-bromo-2-fluorophenol (5.6 g, 29.5 mmol, 1.2 equiv), and triphenylphosphine (9.0 g, 34.4 mmol, 1.4 equiv) in THF (49 mL) was added DIAD (6.7 mL, 34.4 mmol, 1.4 equiv) slowly with a syringe. After stirring 1 h, the reaction was diluted with ether and washed with 10% aqueous potassium carbonate and brine. The ether layer was dried (MgSO₄) and concentrated in vacuo. The crude product was purified by silica gel flash chromatography to provide the product (6.2 g, 86%) as a pale orange oil. ¹H NMR (500 MHz, CDCl₃) δ 7.38-7.24 (m, 6H), 7.18 (dt, J=8.7, 2.0 Hz, 1H), 6.83 (t, J=8.7 Hz, 1H), 4.23 (t, J=7.1 Hz, 2H), 3.15 (t, J=7.1 Hz, 2H).

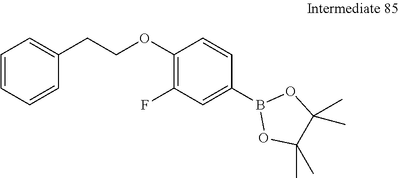 [SMILES: CC1(C)OB(c2ccc(OCCc3ccccc3)c(F)c2)OC1(C)C]

### 2-(3-Fluoro-4-phenethoxyphenyl)-4,4,5,5-tetramethyl-1,3,2-dioxaborolane

A solution of 4-bromo-2-fluoro-1-phenethoxybenzene (1.46 g, 4.95 mmol, 1 equiv), 4,4,4′,4′,5,5,5′,5′-octamethyl-2,2′-bi(1,3,2-dioxaborolane) (1.88 g, 7.42 mmol, 1.5 equiv), Pd(dppf)Cl₂ (0.36 g, 0.495 mmol, 0.1 equiv), and KOAc (1.46 g, 14.8 mmol, 3 equiv) in dioxane (25 mL) was heated at 80° C. for 18 h. After cooling to ambient temperature, the reaction was diluted with EtOAc and washed with brine. The organic layer was dried (Na₂SO₄) and concentrated in vacuo. The crude product was purified by silica gel flash chromatography (0-60% EtOAc in hexane) to provide the product (1.20 g, 71%) as a viscous yellow solid. ¹H NMR (500 MHz, CDCl₃) δ 7.54-7.48 (m, 2H), 7.39-7.29 (m, 5H), 6.94 (t, J=8.0 Hz, 1H), 4.28 (t, J=7.3 Hz, 2H), 3.17 (t, J=7.2 Hz, 2H), 1.35 (s, 12H). LCMS (M+1): 343.05.

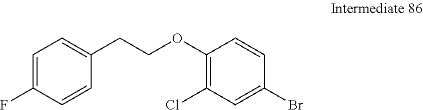 [SMILES: Fc1ccc(CCOc2ccc(Br)cc2Cl)cc1]

### 4-Bromo-2-chloro-1-(4-fluorophenethoxy)benzene

To a solution of 2-(4-fluorophenyl)ethanol (1.0 g, 7.13 mmol, 1 equiv) in DMF (24 mL) was added KOtBu (1.12 g, 9.99 mmol, 1.4 equiv) and 4-bromo-2-chloro-1-fluorobenzene (1.04 mL, 8.56 mmol, 1.2 equiv). After stirring 3 h, the reaction was dilute with ether. The ether solution was washed with water, brine, dried (Na₂SO₄), and concentrated in vacuo. The crude product was purified by silica gel flash chromatography (0-30% EtOAc in hexane) to provide the product (0.93 g, 40%) as a colorless oil. ¹H NMR (500 MHz, CDCl₃) δ 7.37-7.22 (m, 4H), 7.03 (t, J=8.7 Hz, 2H), 6.83-6.68 (m, 1H), 4.18 (t, J=6.6 Hz, 2H), 3.13 (t, J=6.6 Hz, 2H).

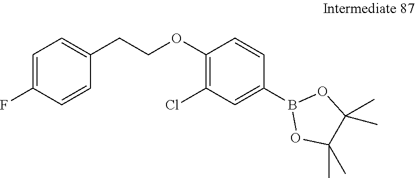 [SMILES: CC1(C)OB(c2ccc(OCCc3ccc(F)cc3)c(Cl)c2)OC1(C)C]

### 2-(3-Chloro-4-(4-fluorophenethoxy)phenyl)-4,5,5-tetramethyl-1,3,2-dioxaborolane

A solution of 4-bromo-2-chloro-1-(4-fluorophenethoxy)benzene (0.93 g, 2.82 mmol, 1 equiv), 4,4,4′,4′,5,5,5′,5′-octamethyl-2,2′-bi(1,3,2-dioxaborolane) (1.08 g, 4.23 mmol, 1.5 equiv), Pd(dppf)Cl₂ (0.21 g, 0.282 mmol, 0.1 equiv), and KOAc (0.83 g, 8.46 mmol, 3 equiv) in dioxane (14 mL) was heated at 80° C. for 18 h. After cooling to ambient temperature, the reaction was diluted with EtOAc and washed with brine. The organic layer was dried (Na₂SO₄) and concentrated in vacuo. The crude product was purified by silica gel flash chromatography (0-60% EtOAc in hexane) to provide the product (0.70 g, 66%) as a white solid. ¹H NMR (500 MHz, CDCl₃) δ 7.82 (d, J=1.3 Hz, 1H), 7.65 (dd, J=8.2, 1.4 Hz, 1H), 7.32 (dd, J=8.5, 5.5 Hz, 2H), 7.02 (t, J=8.7 Hz, 2H), 6.89 (d, J=8.2 Hz, 1H), 4.24 (t, J=6.8 Hz, 2H), 3.15 (t, J=6.8 Hz, 2H), 1.35 (s, 12H). LCMS (M+1): 377.0.

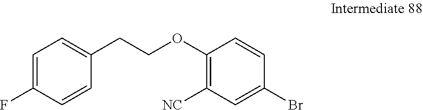 [SMILES: N#Cc1cc(Br)ccc1OCCc1ccc(F)cc1]

### 5-Bromo-2-(4-fluorophenethoxy)benzonitrile

To a solution of 2-(4-fluorophenyl)ethanol (2.57 g, 12.8 mmol, 1 equiv) in DMF (24 mL) was added KOtBu (2.02 g, 18.0 mmol, 1.4 equiv) and 5-bromo-2-fluorobenzonitrile (1.80 g, 12.8 mmol, 1 equiv). After stirring 3 h, the reaction was dilute with ether. The ether solution was washed with water, brine, dried (Na₂SO₄), and concentrated in vacuo. The crude product was purified by silica gel flash chromatography (0-30% EtOAc in hexane) to provide the product (2.24 g, 55%) as a pale yellow oil. ¹H NMR (500 MHz, CDCl₃) δ 7.67 (d, J=2.4 Hz, 1H), 7.61 (dd, J=9.0, 2.5 Hz, 1H), 7.32 (t, J=6.7 Hz, 2H), 7.04 (t, J=8.7 Hz, 2H), 6.82 (d, J=9.0 Hz, 1H), 4.23 (t, J=6.5 Hz, 2H), 3.15 (t, J=6.5 Hz, 2H), 1.60-1.50 (m, 5H).

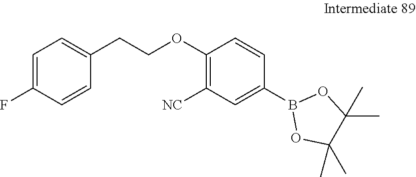 [SMILES: CC1(C)OB(c2ccc(OCCc3ccc(F)cc3)c(C#N)c2)OC1(C)C]

### 2-(4-Fluorophenethoxy)-5-(4,4,5,5-tetramethyl-1,3,2-dioxaborolan-2-yl)benzonitrile

A solution of 5-bromo-2-(4-fluorophenethoxy)benzonitrile (2.0 g, 6.25 mmol, 1 equiv), 4,4,4′,4′,5,5,5′,5′-octamethyl-2,2′-bi(1,3,2-dioxaborolane) (2.4 g, 9.37 mmol, 1.5 equiv), Pd(dppf)Cl₂ (0.48 g, 0.625 mmol, 0.1 equiv), and KOAc (1.84 g, 18.7 mmol, 3 equiv) in dioxane (31 mL) was heated at 80° C. for 18 h. After cooling to ambient temperature, the reaction was diluted with EtOAc and washed with brine. The organic layer was dried (Na₂SO₄) and concentrated in vacuo. The crude product was purified by silica gel flash chromatography (0-60% EtOAc in hexane) to provide the product (2.1 g, 92%) as a viscous yellow oil. ¹H NMR (500 MHz, CDCl₃) δ 8.02 (d, J=1.4 Hz, 1H), 7.92 (dd, J=8.4, 1.7 Hz, 1H), 7.33 (dd, J=8.7, 5.4 Hz, 2H), 7.04 (t, J=8.7 Hz, 2H), 6.91 (d, J=8.5 Hz, 1H), 4.27 (t, J=6.7 Hz, 2H), 3.16 (t, J=6.6 Hz, 2H), 1.35 (s, 12H). LCMS (M+1): 368.05.

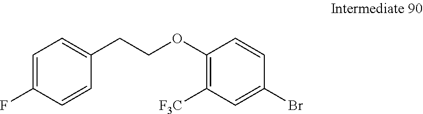 [SMILES: Fc1ccc(CCOc2ccc(Br)cc2C(F)(F)F)cc1]

### 4-Bromo-1-(4-fluorophenethoxy)-2-(trifluoromethyl)benzene

To a solution of 2-(4-fluorophenyl)ethanol (1.02 g, 7.31 mmol, 1.2 equiv) in DMF (20 mL) was added KOtBu (0.96 g, 5.99 mmol, 1.4 equiv) and 4-bromo-1-fluoro-2-(trifluoromethyl)benzene (1.48 g, 6.09 mmol, 1.2 equiv). After stirring 2 h, the reaction was dilute with ether. The ether solution was washed with water, brine, dried (Na₂SO₄), and concentrated in vacuo. The crude product was purified by silica gel flash chromatography (0-30% EtOAc in hexane) to provide the product (1.36 g, 62%) as a white solid. ¹H NMR (500 MHz, CDCl₃) δ 7.69 (d, J=2.5 Hz, 1H), 7.57 (dd, J=8.8, 2.5 Hz, 1H), 7.28-7.25 (m, 2H), 7.02 (t, J=8.3 Hz, 2H), 6.85 (d, J=8.8 Hz, 1H), 4.21 (t, J=6.5 Hz, 2H), 3.12 (t, J=6.5 Hz, 2H).

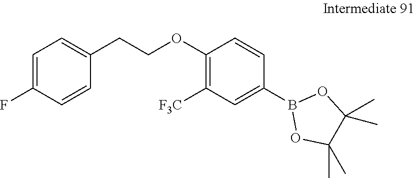 [SMILES: CC1(C)OB(c2ccc(OCCc3ccc(F)cc3)c(C(F)(F)F)c2)OC1(C)C]

### 2-(4-(4-Fluorophenethoxy)-3-(trifluoromethyl)phenyl)-4,5,5-tetramethyl-1,3,2-dioxaborolane

A solution of 4-bromo-1-(4-fluorophenethoxy)-2-(trifluoromethyl)benzene (1.36 g, 3.75 mmol, 1 equiv), 4,4,4′,4′,5,5,5′,5′-octamethyl-2,2′-bi(1,3,2-dioxaborolane) (1.43 g, 5.62 mmol, 1.5 equiv), Pd(dppf)Cl₂ (0.274 g, 0.375 mmol, 0.1 equiv), and KOAc (1.10 g, 11.2 mmol, 3 equiv) in dioxane (19 mL) was heated at 80° C. for 18 h. After cooling to ambient temperature, the reaction was diluted with EtOAc and washed with brine. The organic layer was dried (Na₂SO₄) and concentrated in vacuo. The crude product was purified by silica gel flash chromatography (0-60% EtOAc in hexane) to provide the product (1.20 g, 78%) as a viscous yellow oil. ¹H NMR (500 MHz, CDCl₃) δ 8.02 (s, 1H), 7.91 (d, J=8.4 Hz, 1H), 7.31-7.27 (m, 2H), 7.02 (t, J=8.7 Hz, 2H), 6.95 (d, J=8.4 Hz, 1H), 4.26 (t, J=6.6 Hz, 2H), 3.13 (t, J=6.5 Hz, 2H), 1.36 (s, 12H). ¹⁹F NMR (471 MHz, CDCl₃) δ −62.17 (s, 3F), −116.70 (br s, 1F). LCMS (M+1): 411.0.

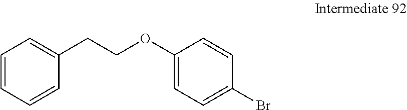 [SMILES: Brc1ccc(OCCc2ccccc2)cc1]

### 1-Bromo-4-phenethoxybenzene

To a 100 mL round bottom flask equipped with a stir bar was added 4-bromophenol (1.00 g, 5.78 mmol), 2-phenylethan-1-ol (706 mg, 5.78 mmol), triphenylphosphine (1.82 g, 6.94 mmol) and THF (30 ml). To the stirring solution was added diethyl (E)-diazene-1,2-dicarboxylate (1.09 ml, 6.94 mmol) dropwise. The solution was stirred at r.t. for 2 hrs. The reaction solution was concentrated in vacuo and the resulting oil was diluted with a min of acetone, then concentrated onto Celite in vacuo. The resulting powder was subjected to silica gel chromatography (80 g column, 0-50% EtOAc:Hex over 10 CVs) to afford the product 1-bromo-4-phenethoxybenzene (1.05 g, 3.79 mmol, 65.5% yield) as a clear oil. ¹H NMR (500 MHz, CHLOROFORM-d) δ 7.43-7.30 (m, 7H), 6.80 (d, J=8.1 Hz, 2H), 4.17 (t, J=7.1 Hz, 2H), 3.11 (t, J=7.1 Hz, 2H)

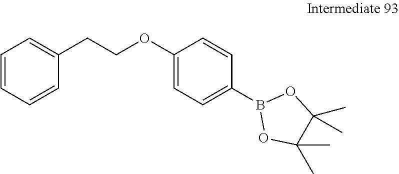 [SMILES: CC1(C)OB(c2ccc(OCCc3ccccc3)cc2)OC1(C)C]

### 4,4,5,5-Tetramethyl-2-(4-phenethoxyphenyl)-1,3,2-dioxaborolane

1-Bromo-4-phenethoxybenzene (1.0 g, 3.6 mmol), BISPIN (1.0 g, 3.9 mmol), PdCl₂(dppf) (0.132 g, 0.180 mmol), potassium acetate (1.06 g, 10.8 mmol) were combined in 1,4 dioxane (18 ml) at rt, degassed and backfilled with N₂, and warmed to 90° C. The reaction was allowed to stir overnight. The reaction was concentrated, adsorbed onto celite and was purified on silica gel (Biotage, EtOAc/hexanes gradient, 0-100% over 10 CVs) to afford 4,4,5,5-tetramethyl-2-(4-phenethoxyphenyl)-1,3,2-dioxaborolane (1.0 g, 3.08 mmol, 85% yield). LCMS Method 4: retention time=1.78 min.; observed ion=325.05. ¹H NMR (500 MHz, CHLOROFORM-d) δ 7.76 (d, J=7.9 Hz, 2H), 7.41-7.30 (m, 4H), 7.28-7.24 (m, 1H), 6.91 (d, J=7.9 Hz, 2H), 4.23 (t, J=7.2 Hz, 2H), 3.13 (t, J=7.2 Hz, 2H), 1.35 (s, 12H).

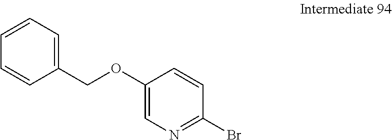 [SMILES: Brc1ccc(OCc2ccccc2)cn1]

### 5-(Benzyloxy)-2-bromopyridine

To a 1 L round bottom flask equipped with a large stir bar was added 6-bromopyridin-3-ol (24.69 g, 142 mmol), benzyl alcohol (15.42 mL, 149 mmol), triphenylphosphine (39.1 g, 149 mmol) and THF (600 mL). The flask was placed in a r.t. water bath. To the stirred solution was added in six portions DIAD (29.0 mL, 149 mmol). The internal temperature increased from 20 deg to 35 deg C., and was 35 deg C. at the completion of the addition. After stirring for 18 h the reaction solution was concentrated in vacuo to afford a liquid residue. The material was diluted with hexane:Et₂O (1:1, 850 mL). A precipitate was immediately formed. The mixture was stirred for 5 minutes, then the liquid was decanted and reserved. The solids were treated with Et₂O (200 mL), and the mixture was stirred for 5 minutes. The solution was diluted with hexanes (200 mL), and the mixture was then stirred for 5 minutes. The mixture and the reserved solution were combined and filtered through a fine-fritted vacuum funnel. The filtrate was concentrated in vacuo. The resulting residue was diluted with a small amount of acetone and then concentrated onto Celite in vacuo. The resulting powder was subjected to SiO₂ chromatography (330 g SiO₂ column, hexanes:EtOAc 100:0→80:20) to afford 5-(benzyloxy)-2-bromopyridine as a colorless, crystalline solid (24.84 g, 66%). ¹H NMR (400 MHz, CHLOROFORM-d) δ 8.16 (d, J=3.2 Hz, 1H), 7.45-7.36 (m, 6H), 7.18 (dd, J=8.6, 3.2 Hz, 1H), 5.12 (s, 2H).

Alternative Procedure:

To a stirred solution of 6-bromopyridin-3-ol (100 g, 575 mmol), K₂CO₃ (119 g, 862 mmol) in acetone (1 L) was added benzyl bromide (0.075 L, 632 mmol). The mixture was stirred for 3 h at 80° C. LCMS showed completion of reaction. Then, the mixture was cooled to 20° C. and poured into water (250 mL). The precipitate was filtered, taken up in in DCM and washed with sat. NaHCO₃ (50 mL), water (50 mL), brine (50 mL) and concentrated to afford 5-(benzyloxy)-2-bromopyridine (120 g, 454 mmol, 79% yield) as a off-white solid.

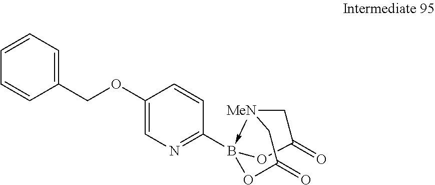

### (5-(Benzyloxy)pyridin-2-yl)boronic acid MIDA ester

To a dry 250 mL round bottom flask equipped with a large stir bar and charged with 5-(benzyloxy)-2-bromopyridine (21.8527 g, 83 mmol) was added THF (150 ml) and triisopropyl borate (19.40 ml, 84 mmol). The flask was sealed with a rubber septum, then the solution was sparged with N₂ for 5 min. The flask was cooled in a −78° C. bath. To the solution was added dropwise over 30 min n-butyllithium in hexanes (33.4 ml, 84 mmol). The cold bath was removed and the solution was allowed to slowly warm to r.t. with stirring. After 2 h the solution was transferred to a pressure-equalizing addition funnel. The addition funnel was fitted onto the center neck of a 3-neck 250 mL flask equipped with a large stir bar was charged with N-methyliminodiacetic acid (24.35 g, 165 mmol) and DMSO (150 ml). A side neck was fitted with a thermocouple. The other side neck was fitted with a water-cooled short-path distillation apparatus collecting into a 250 mL round bottom flask and vented to a bubbler. The addition funnel was capped with a gas adapter connected to a low-volume stream of N₂ gas. The 3-neck flask was heated with an oil bath (150° C.). Once the DMSO solution had reached 115-120° C. the boronate solution was added dropwise at a rate necessary to maintain an internal temp of 115-120° C. The blue boronate solution immediately becomes a red/amber color upon contacting the DMSO solution. The reaction mixture is a deep amber solution. Upon completion of the addition the receiver flask containing THF was exchanged for an empty 200 mL round bottom flask. The bubbler line connected to the vacuum arm of the distillation apparatus was exchanged for a controlled vacuum source. The N₂ source feeding into the addition funnel was closed. The system was placed under vacuum, slowly ramping to 30 Torr. The receiver flask was emptied, then the vacuum was slowly ramped to 2 Torr. The bath temperature was set to 125° C. and the pressure was maintained at 2 Torr. When the majority of DMSO had been removed the flask was opened to ambient atmosphere. To the flask was added MeCN (100 mL). Heating was maintained until the solvent had reached reflux, then heating was stopped. To the hot mixture was added Celite. The mixture was concentrated in vacuo to afford a clumpy solid which was subjected to SiO₂ chromatography (EtOAc:MeCN 100:0→0:100) to afford the desired product as a colorless solid. This material was dissolved/suspended in MeCN (100 mL), then was diluted with Et₂O (400 mL). The crystalline solid was collected via vacuum filtration. The solids were dried under high vacuum to afford (5-(benzyloxy)pyridin-2-yl)boronic acid MIDA ester as a colorless, fine crystalline solid (5.81 g, 21%). ¹H NMR (500 MHz, ACETONITRILE-d₃) δ 8.50-8.42 (m, 1H), 7.56 (d, J=8.4 Hz, 1H), 7.49-7.44 (m, 2H), 7.44-7.38 (m, 2H), 7.38-7.33 (m, 1H), 7.31 (dd, J=8.4, 2.9 Hz, 1H), 5.16 (s, 2H), 4.10-4.04 (m, 2H), 3.98-3.92 (m, 2H), 2.54 (s, 3H).

Simplified Procedure:

To a solution of 5-(benzyloxy)-2-bromopyridine (98 g, 371 mmol) and triisopropyl borate (77 g, 408 mmol) in THF (800 mL) was added n-butyllithium (193 mL, 482 mmol) at −78° C. and stirred for 20 min at −78° C. Then, the reaction mixture was stirred at rt for 3 h. The reaction mixture was added to a solution of 2,2′-(2,2′-(methylazanediyl)diacetic acid (109 g, 742 mmol) in DMSO (800 mL) at 115-120° C. (internal temperature). Then, the THF and DMSO was distilled off over 2 h to remove as much solvent as possible. The reaction flask was cooled, diluted with 3000 mL of ethyl acetate and washed with water (2000 mL×2), dried and concentrated. The residue was purified by silica gel chromatography to give 2-(5-(benzyloxy)pyridin-2-yl)-6-methyl-1,3,6,2-dioxazaborocane-4,8-dione (41 g, 120 mmol, 32.2% yield) as pale solid.

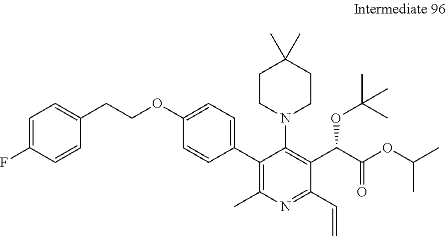 [SMILES: C=Cc1nc(C)c(-c2ccc(OCCc3ccc(F)cc3)cc2)c(N2CCC(C)(C)CC2)c1[C@H](OC(C)(C)C)C(=O)OC(C)C]

### (S)-Isopropyl 2-(tert-butoxy)-2-(4-(4,4-dimethylpiperidin-1-yl)-5-(4-(4-fluorophenethoxy)phenyl)-6-methyl-2-vinylpyridin-3-yl)acetate

A mixture of (S)-isopropyl 2-(5-bromo-4-(4,4-dimethylpiperidin-1-yl)-6-methyl-2-vinylpyridin-3-yl)-2-(tert-butoxy)acetate (18 mg, 0.037 mmol), (4-(4-fluorophenethoxy)phenyl)boronic acid (14.58 mg, 0.056 mmol), 2-dicyclohexylphosphino-2′,6′-dimethoxybiphenyl (4.60 mg, 0.011 mmol), and potassium phosphate tribasic (59.5 mg, 0.280 mmol) in 1,4-dioxane (623 μl) and water (125 μl) was bubbled with N₂ for 10 minutes. Pd(OAc)₂ (1.259 mg, 5.61 μmol) was then added and the reaction vessel sealed under positive pressure of N₂. The reaction was heated at 80° C. for 2 h. The reaction was diluted with water and extracted with EtOAc. The organic layer was washed with brine, collected, dried over MgSO₄, filtered and volatiles evaporated to afford the crude product. The crude product was purified on silica gel (4 g column, 5-25% EtOAc:Hex) to afford the product (S)-isopropyl 2-(tert-butoxy)-2-(4-(4,4-dimethylpiperidin-1-yl)-5-(4-(4-fluorophenethoxy)phenyl)-6-methyl-2-vinylpyridin-3-yl)acetate (11 mg, 0.018 mmol, 47.7% yield) as a lightly colored oil. LCMS (M+1)=617.7.

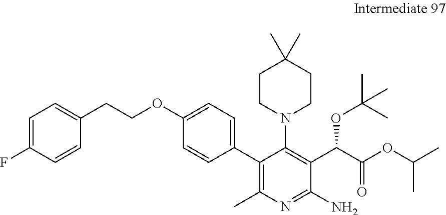 [SMILES: Cc1nc(N)c([C@H](OC(C)(C)C)C(=O)OC(C)C)c(N2CCC(C)(C)CC2)c1-c1ccc(OCCc2ccc(F)cc2)cc1]

### (S)-Isopropyl 2-(2-amino-4-(4,4-dimethylpiperidin-1-yl)-5-(4-(4-fluorophenethoxy)phenyl)-6-methylpyridin-3-yl)-2-(tert-butoxy)acetate

A mixture of (S)-isopropyl 2-(2-amino-5-bromo-4-(4,4-dimethylpiperidin-1-yl)-6-methylpyridin-3-yl)-2-(tert-butoxy)acetate (200 mg, 0.425 mmol), (4-(4-fluorophenethoxy)phenyl)boronic acid (166 mg, 0.638 mmol), 2-dicyclohexylphosphino-2′,6′-dimethoxybiphenyl (34.9 mg, 0.085 mmol) and potassium phosphate tribasic (677 mg, 3.19 mmol) in 1,4-dioxane (7086 μl) and water (1417 μl), then bubbled with nitrogen for 10 minutes. Pd(OAc)₂ (9.54 mg, 0.043 mmol) was added to the reaction and heated at 80° C. for 2 h. The reaction was diluted with water and extracted with EtOAc. The organic layer was washed with brine, collected, dried over MgSO₄, filtered and volatiles evaporated to afford the crude product. The crude product was purified on silica gel (40 g column, 5-50% EtOAc:Hex) to afford the product (S)-isopropyl 2-(2-amino-4-(4,4-dimethylpiperidin-1-yl)-5-(4-(4-fluorophenethoxy)phenyl)-6-methylpyridin-3-yl)-2-(tert-butoxy)acetate (150 mg, 0.248 mmol, 58.2% yield) as a yellow solid. ¹H NMR (500 MHz, CDCl₃) δ 7.30 (br d, J=2.4 Hz, 2H), 7.13 (dd, J=8.7, 2.0 Hz, 1H), 7.10-7.06 (m, 1H), 7.04 (t, J=8.7 Hz, 2H), 6.97-6.91 (m, 2H), 6.02 (s, 1H), 5.19 (br s, 2H), 5.07 (dt, J=12.5, 6.3 Hz, 1H), 4.23 (td, J=6.9, 3.3 Hz, 2H), 3.12 (t, J=6.9 Hz, 1H), 3.17 (br s, 1H), 2.85 (br t, J=11.3 Hz, 1H), 2.36-2.28 (m, 1H), 2.15 (br t, J=11.2 Hz, 1H), 2.04 (s, 3H), 1.40-1.28 (m, 2H), 1.26 (d, J=6.3 Hz, 3H), 1.25-1.25 (m, 1H), 1.24 (s, 9H), 1.21 (d, J=6.1 Hz, 3H), 1.19-1.09 (m, 2H), 0.90 (s, 3H), 0.64 (s, 3H). LCMS (M+1)=606.7.

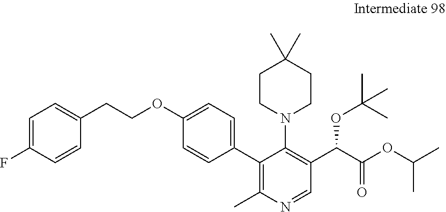 [SMILES: Cc1ncc([C@H](OC(C)(C)C)C(=O)OC(C)C)c(N2CCC(C)(C)CC2)c1-c1ccc(OCCc2ccc(F)cc2)cc1]

### (S)-Isopropyl 2-(tert-butoxy)-2-(4-(4,4-dimethylpiperidin-1-yl)-5-(4-(4-fluorophenethoxy)phenyl)-6-methylpyridin-3-yl)acetate

To a solution of (S)-isopropyl 2-(5-bromo-4-(4,4-dimethylpiperidin-1-yl)-6-methylpyridin-3-yl)-2-(tert-butoxy)acetate (1 g, 2.196 mmol), (4-(4-fluorophenethoxy)phenyl)boronic acid (0.857 g, 3.29 mmol), 2-dicyclohexylphosphino-2′,6′-dimethoxybiphenyl (0.180 g, 0.439 mmol), and potassium phosphate tribasic (3.50 g, 16.47 mmol) in 1,4-dioxane (36.6 ml) and water (7.32 ml) under N₂ was added Pd(OAc)₂ (0.049 g, 0.220 mmol). The reaction was heated at 80° C. for 2 h. The reaction was cooled to RT and diluted with water and extracted with EtOAc. The organic layer was washed with brine, collected, dried over MgSO₄, filtered and volatiles evaporated to afford the crude product. The crude product was purified on silica gel (40 g column, 5-50% EtOAc:Hex) to give the product (S)-isopropyl 2-(tert-butoxy)-2-(4-(4,4-dimethylpiperidin-1-yl)-5-(4-(4-fluorophenethoxy)phenyl)-6-methylpyridin-3-yl)acetate (1.15 g, 1.947 mmol, 89% yield) as a brown oil. ¹H NMR (400 MHz, CDCl₃) δ 8.68 (s, 1H), 7.30 (d, J=5.4 Hz, 1H), 7.27 (br. s., 1H), 7.18-7.11 (m, 1H), 7.09-7.01 (m, 3H), 6.98 (d, J=8.8 Hz, 2H), 5.52 (s, 1H), 5.04 (dt, J=12.6, 6.2 Hz, 1H), 4.24 (t, J=7.0 Hz, 2H), 3.13 (t, J=7.0 Hz, 2H), 2.93 (br. s., 1H), 2.65 (br. s., 1H), 2.51 (d, J=7.1 Hz, 1H), 2.39 (br. s., 1H), 2.23 (s, 3H), 1.41-1.31 (m, 1H), 1.27 (d, J=6.1 Hz, 4H), 1.25 (s, 9H), 1.23 (d, J=6.1 Hz, 4H), 1.16-1.02 (m, 1H), 0.86 (br. s., 3H), 0.72 (br. s., 3H). LCMS (M+1)=591.4.

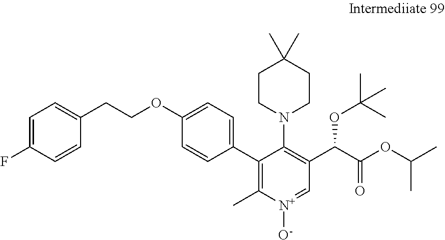 [SMILES: Cc1c(-c2ccc(OCCc3ccc(F)cc3)cc2)c(N2CCC(C)(C)CC2)c([C@H](OC(C)(C)C)C(=O)OC(C)C)c[n+]1[O-]]

### (S)-5-(1-(tert-Butoxy)-2-isopropoxy-2-oxoethyl)-4-(4,4-dimethylpiperidin-1-yl)-3-(4-(4-fluorophenethoxy)phenyl)-2-methylpyridine 1-oxide

To a stirred solution of (S)-isopropyl 2-(tert-butoxy)-2-(4-(4,4-dimethylpiperidin-1-yl)-5-(4-(4-fluorophenethoxy)phenyl)-6-methylpyridin-3-yl)acetate (1.15 g, 1.947 mmol) in DCM (10 ml) was added 77% mCPBA (0.654 g, 2.92 mmol) at rt over 5 min. After 4 h, the reaction mixture was washed with sat. Na₂CO₃ (3×25 mL), dried (MgSO₄), filtered and concentrated to give (S)-5-(1-(tert-butoxy)-2-isopropoxy-2-oxoethyl)-4-(4,4-dimethylpiperidin-1-yl)-3-(4-(4-fluorophenethoxy)phenyl)-2-methylpyridine 1-oxide (1.1 g, 1.813 mmol, 93% yield) which was used in the next step without purification. ¹H NMR (500 MHz, CDCl₃) δ 8.55 (s, 1H), 7.31-7.29 (m, 1H), 7.28-7.27 (m, 1H), 7.13-6.97 (m, 6H), 5.40 (s, 1H), 5.06-4.98 (m, 1H), 4.24 (t, J=6.9 Hz, 2H), 3.13 (t, J=6.9 Hz, 2H), 2.70 (br. s., 1H), 2.58-2.44 (m, 2H), 2.39-2.29 (m, 1H), 2.22 (s, 3H), 1.53-1.35 (m, 2H), 1.27 (s, 3H), 1.25 (s, 9H), 1.24 (s, 3H), 1.19-1.05 (m, 2H), 0.91 (br. s., 3H), 0.64 (br. s., 3H). LCMS (M+1)=607.4.

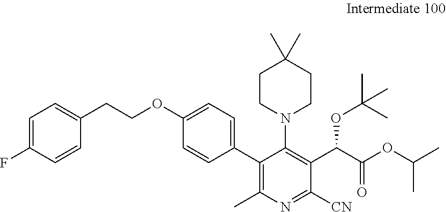 [SMILES: Cc1nc(C#N)c([C@H](OC(C)(C)C)C(=O)OC(C)C)c(N2CCC(C)(C)CC2)c1-c1ccc(OCCc2ccc(F)cc2)cc1]

### (S)-Isopropyl 2-(tert-butoxy)-2-(2-cyano-4-(4,4-dimethylpiperidin-1-yl)-5-(4-(4-fluorophenethoxy)phenyl)-6-methylpyridin-3-yl)acetate

To a solution of (S)-5-(1-(tert-butoxy)-2-isopropoxy-2-oxoethyl)-4-(4,4-dimethylpiperidin-1-yl)-3-(4-(4-fluorophenethoxy)phenyl)-2-methylpyridine 1-oxide (25 mg, 0.041 mmol) and trimethylsilanecarbonitrile (5.67 μl, 0.045 mmol) in acetonitrile (0.5 mL) in a 1 dram vial was added dimethylcarbamoyl chloride (4.17 μl, 0.045 mmol) and allowed to stir at RT for 18 hr. The reaction was monitored via LCMS. The reaction was heated to 40° C. for 1 hr then to 55° C. for another hr to try to push to full conversion. The reaction material was transferred to a separatory funnel and washed with sat. sodium hydrogen carbonate solution and extracted with EtOAc. The organic layer was washed with brine, collected, dried over MgSO₄, filtered and evaporated to afford the crude product (S)-isopropyl 2-(tert-butoxy)-2-(2-cyano-4-(4,4-dimethylpiperidin-1-yl)-5-(4-(4-fluorophenethoxy)phenyl)-6-methylpyridin-3-yl)acetate (20 mg, 0.032 mmol, 79% yield). LCMS (M+1)=616.4.

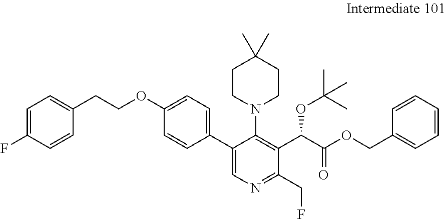 [SMILES: CC1(C)CCN(c2c(-c3ccc(OCCc4ccc(F)cc4)cc3)cnc(CF)c2[C@H](OC(C)(C)C)C(=O)OCc2ccccc2)CC1]

### (S)-Benzyl 2-(tert-butoxy)-2-(4-(4, 4-dimethylpiperidin-1-yl)-2-(fluoromethyl)-5-(4-(4-fluorophenethoxy)phenyl)pyridin-3-yl)acetate

A mixture of (S)-benzyl 2-(5-bromo-4-(4,4-dimethylpiperidin-1-yl)-2-(fluoromethyl)pyridin-3-yl)-2-(tert-butoxy)acetate (83 mg, 0.159 mmol), (4-(4-fluorophenethoxy)phenyl)boronic acid (62.1 mg, 0.239 mmol), 2-dicyclohexylphosphino-2′,6′-dimethoxybiphenyl (13.07 mg, 0.032 mmol), and potassium phosphate tribasic (253 mg, 1.194 mmol) in 1,4-dioxane (2653 μl) and water (531 μl) was bubbled with N₂ for 10 minutes. Pd(OAc)₂ (3.57 mg, 0.016 mmol) was added and the reaction was kept under positive pressure of N₂ for the duration of the reaction. The reaction was heated at 80° C. for 1 h. The reaction was cooled to RT and diluted with water and EtOAc. The organic layer was washed with brine, collected, dried over MgSO₄, filtered and volatiles evaporated to afford the crude product. The crude product was purified via silica gel (24 g column, 5-40% EtOAc:Hex) to afford the product (S)-benzyl 2-(tert-butoxy)-2-(4-(4,4-dimethylpiperidin-1-yl)-2-(fluoromethyl)-5-(4-(4-fluorophenethoxy)phenyl)pyridin-3-yl)acetate (43 mg, 0.065 mmol, 41.1% yield) as a light colored thick oil. LCMS (M+1)=657.3.

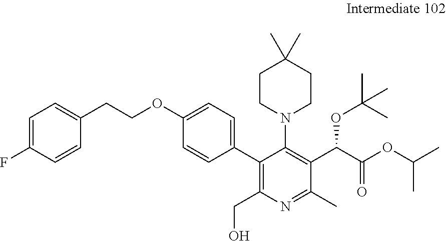 [SMILES: Cc1nc(CO)c(-c2ccc(OCCc3ccc(F)cc3)cc2)c(N2CCC(C)(C)CC2)c1[C@H](OC(C)(C)C)C(=O)OC(C)C]

### (S)-Isopropyl 2-(tert-butoxy)-2-(4-(4, 4-dimethylpiperidin-1-yl)-5-(4-(4-fluorophenethoxy)phenyl)-6-(hydroxymethyl)-2-methylpyridin-3-yl)acetate

A mixture of (S)-isopropyl 2-(5-bromo-4-(4,4-dimethylpiperidin-1-yl)-6-(hydroxymethyl)-2-methylpyridin-3-yl)-2-(tert-butoxy)acetate (1 g, 2.060 mmol), (4-(4-fluorophenethoxy)phenyl)boronic acid (0.804 g, 3.09 mmol), 2-dicyclohexylphosphino-2′,6′-dimethoxybiphenyl (0.169 g, 0.412 mmol), and potassium phosphate tribasic (3.28 g, 15.45 mmol) in 1,4-dioxane (34.3 ml) and water (6.87 ml) was bubbled with N₂ for 10 minutes. Pd(OAc)₂ (0.046 g, 0.206 mmol) was added and the reaction vessel capped under positive pressure of N₂. The reaction was heated at 80° C. for 2 h. The reaction was diluted with water and extracted with EtOAc. The organic layer was washed with brine, collected, dried over MgSO₄, filtered and volatiles evaporated to afford the crude product. The crude product was purified on silica gel (40 g column, 5-50% EtOAc:Hex) to afford the product (S)-isopropyl 2-(tert-butoxy)-2-(4-(4,4-dimethylpiperidin-1-yl)-5-(4-(4-fluorophenethoxy)phenyl)-6-(hydroxymethyl)-2-methylpyridin-3-yl)acetate (1.15 g, 1.852 mmol, 90% yield) as a brown oil. ¹H NMR (500 MHz, CDCl₃) δ 7.31-7.29 (m, 2H), 7.13 (dd, J=8.3, 2.0 Hz, 1H), 7.09-7.02 (m, 3H), 7.00-6.93 (m, 2H), 6.04 (br s, 1H), 5.11 (dt, J=12.5, 6.2 Hz, 1H), 4.42 (d, J=15.4 Hz, 1H), 4.26-4.20 (m, 2H), 4.08 (d, J=15.4 Hz, 1H), 3.30-3.18 (m, 1H), 3.14 (t, J=6.9 Hz, 2H), 2.95-2.83 (m, 1H), 2.65 (s, 3H), 2.31 (br s, 1H), 2.19-2.09 (m, 1H), 1.58 (br s, 4H), 1.26 (d, J=6.3 Hz, 3H), 1.23 (d, J=6.3 Hz, 3H), 1.20 (s, 9H), 0.95-0.85 (m, 3H), 0.68 (br s, 3H). LCMS (M+1)=621.7.

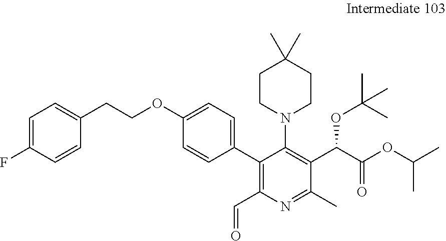 [SMILES: Cc1nc(C=O)c(-c2ccc(OCCc3ccc(F)cc3)cc2)c(N2CCC(C)(C)CC2)c1[C@H](OC(C)(C)C)C(=O)OC(C)C]

### (S)-Isopropyl 2-(tert-butoxy)-2-(4-(4, 4-dimethylpiperidin-1-yl)-5-(4-(4-fluorophenethoxy)phenyl)-6-formyl-2-methylpyridin-3-yl)acetate

To a stirred solution of (S)-isopropyl 2-(tert-butoxy)-2-(4-(4,4-dimethylpiperidin-1-yl)-5-(4-(4-fluorophenethoxy)phenyl)-6-(hydroxymethyl)-2-methylpyridin-3-yl)acetate (0.960 g, 1.546 mmol) in DCM (14.06 ml) and acetonitrile (1.406 ml) was added Dess-Martin Periodinane (0.984 g, 2.320 mmol) at once at rt. After 4 h, the reaction mixture was diluted with ether (25 mL), washed with 1M NaOH (2×25 ml), brine (25 mL), dried (MgSO₄), filtered and concentrated to afford the product (S)-isopropyl 2-(tert-butoxy)-2-(4-(4,4-dimethylpiperidin-1-yl)-5-(4-(4-fluorophenethoxy)phenyl)-6-formyl-2-methylpyridin-3-yl)acetate (829 mg, 1.340 mmol, 87% yield). ¹H NMR (500 MHz, CDCl₃) δ 9.84 (s, 1H), 7.32-7.29 (m, 2H), 7.22 (ddd, J=11.0, 8.6, 2.1 Hz, 2H), 7.08-7.03 (m, 2H), 7.00 (ddd, J=16.3, 8.3, 2.5 Hz, 2H), 6.14-6.03 (m, 1H), 5.13 (dt, J=12.5, 6.3 Hz, 1H), 4.30-4.21 (m, 2H), 3.30-3.20 (m, 1H), 3.14 (t, J=6.9 Hz, 2H), 2.98 (br t, J=11.0 Hz, 1H), 2.72 (s, 3H), 2.31 (br d, J=8.7 Hz, 1H), 2.14 (br d, J=11.0 Hz, 1H), 1.59-1.27 (m, 4H), 1.26 (d, J=6.1 Hz, 3H), 1.24 (d, J=6.1 Hz, 3H), 1.19 (s, 9H), 0.93 (br s, 3H), 0.71 (br s, 3H). LCMS (M+1)=619.3.

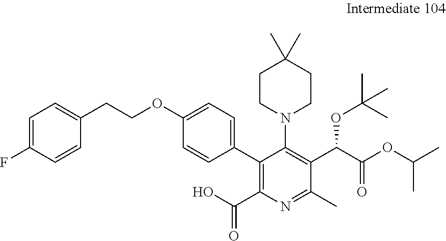 [SMILES: Cc1nc(C(=O)O)c(-c2ccc(OCCc3ccc(F)cc3)cc2)c(N2CCC(C)(C)CC2)c1[C@H](OC(C)(C)C)C(=O)OC(C)C]

### (S)-5-(1-(tert-Butoxy)-2-isopropoxy-2-oxoethyl)-4-(4, 4-dimethylpiperidin-1-yl)-3-(4-(4-fluorophenethoxy)phenyl)-6-methylpicolinic acid

To a solution of (S)-isopropyl 2-(tert-butoxy)-2-(4-(4,4-dimethylpiperidin-1-yl)-5-(4-(4-fluorophenethoxy)phenyl)-6-formyl-2-methylpyridin-3-yl)acetate (529 mg, 0.855 mmol) in DMSO (15 ml) was added potassium phosphate monobasic (349 mg, 2.56 mmol) in water (0.75 mL) followed by sodium chlorite (232 mg, 2.56 mmol) in water (1.5 mL) and the mixture was stirred for 3 hr. The reaction was diluted with water and EtOAc. The organic layer was washed with water (2×). The organic layer was then washed with brine dried (MgSO₄), filtered and concentrated to afford the product (S)-5-(1-(tert-butoxy)-2-isopropoxy-2-oxoethyl)-4-(4,4-dimethylpiperidin-1-yl)-3-(4-(4-fluorophenethoxy)phenyl)-6-methylpicolinic acid (536 mg, 0.844 mmol, 99% yield) as a yellow crispy foam. ¹H NMR (500 MHz, CDCl₃) δ 7.30 (br s, 1H), 7.29-7.24 (m, 2H), 7.09-7.00 (m, 3H), 6.99-6.94 (m, 2H), 6.05 (br s, 1H), 5.17-5.08 (m, 1H), 4.24 (td, J=7.0, 1.8 Hz, 2H), 3.29-2.69 (m, 4H), 2.67 (s, 3H), 2.61-2.11 (m, 2H), 1.34-1.15 (m, 19H), 1.00-0.68 (m, 6H). LCMS (M+1)=635.3.

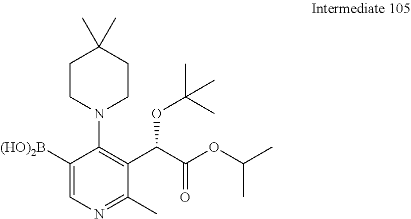 [SMILES: Cc1ncc(B(O)O)c(N2CCC(C)(C)CC2)c1[C@H](OC(C)(C)C)C(=O)OC(C)C]

### (S)-(5-(1-(tert-Butoxy)-2-isopropoxy-2-oxoethyl)-4-(4, 4-dimethylpiperidin-1-yl)-6-methylpyridin-3-yl)boronic acid

To a dry 10 mL Schlenk flask equipped with a stir bar and placed under N₂ was added (S)-isopropyl 2-(5-bromo-4-(4,4-dimethylpiperidin-1-yl)-2-methylpyridin-3-yl)-2-(tert-butoxy)acetate (0.114 g, 0.250 mmol), then placed under vacuum and back filled with N₂ (3 times) with finally being left under positive pressure of N₂. THF (3 mL) was then added followed by pyridine (0.040 mL, 0.501 mmol). The solution was cooled to −78° C. To the solution was added butyllithium (0.100 mL, 0.250 mmol) dropwise upon which the solution turned a light brown color. The solution was stirred for 5 minutes. To the solution was added trimethyl borate (0.112 mL, 1.001 mmol). Stirring was maintained as the −78° C. for 5 minutes and then the bath was removed and slowly warmed to RT and stirred for 18 hr. The reaction mixture was transferred to a 125 mL separatory funnel and was diluted with water (25 mL), then extracted with EtOAc (50 mL). The organic phase was washed with brine, dried over MgSO₄, filtered and volatiles evaporated to afford the product (S)-(5-(1-(tert-butoxy)-2-isopropoxy-2-oxoethyl)-4-(4,4-dimethylpiperidin-1-yl)-6-methylpyridin-3-yl)boronic acid which was used in the subsequent step without purification. LCMS (M+1)=421.1.

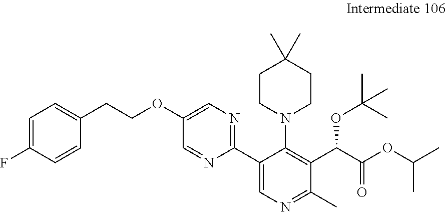 [SMILES: Cc1ncc(-c2ncc(OCCc3ccc(F)cc3)cn2)c(N2CCC(C)(C)CC2)c1[C@H](OC(C)(C)C)C(=O)OC(C)C]

### Isopropyl (S)-2-(tert-butoxy)-2-(4-(4, 4-dimethylpiperidin-1-yl)-5-(5-(4-fluorophenethoxy)pyrimidin-2-yl)-2-methylpyridin-3-yl)acetate

To a 14 mL test tube equipped with a stir and charged with crude (S)-(5-(1-(tert-butoxy)-2-isopropoxy-2-oxoethyl)-4-(4,4-dimethylpiperidin-1-yl)-6-methylpyridin-3-yl)boronic acid (113 mg, 0.269 mmol) and SPhos-Pd-G3 (10.47 mg, 0.013 mmol) was added tribasic potassium phosphate (514 mg, 2.419 mmol) and 2-chloro-5-(4-fluorophenethoxy)pyrimidine (67.9 mg, 0.269 mmol). The flask was sealed with a rubber septum, then was placed under N₂ atm (vac/fill×3). To the flask was added degassed (N₂ bubbling for 5 min) dioxane (1 mL)+water (0.4 mL). The test tube was placed in a 60° C. heating block with stirring. The reaction was stirred for 3 hours at this temperature. The reaction was then cooled to RT and then diluted with water and EtOAc. The organic layer was washed with brine, collected, dried over MgSO₄, filtered and volatiles evaporated to afford the crude material. The crude material was purified via silica gel chromatography (24 g column, 25-100% EtOAc:Hex) to afford the product isopropyl (S)-2-(tert-butoxy)-2-(4-(4,4-dimethylpiperidin-1-yl)-5-(5-(4-fluorophenethoxy)pyrimidin-2-yl)-2-methylpyridin-3-yl)acetate (20 mg, 0.034 mmol, 12.55% yield over 2 steps) as a brown oil. LCMS (M+1)=593.4.

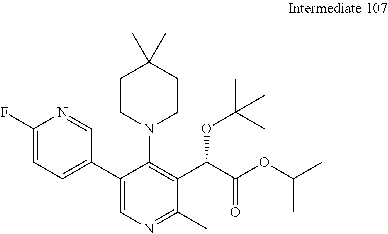 [SMILES: Cc1ncc(-c2ccc(F)nc2)c(N2CCC(C)(C)CC2)c1[C@H](OC(C)(C)C)C(=O)OC(C)C]

### Isopropyl (S)-2-(tert-butoxy)-2-(4-(4, 4-dimethylpiperidin-1-yl)-6′-fluoro-6-methyl-[3, 3′-bipyridin]-5-yl)acetate

A mixture of isopropyl (S)-2-(5-bromo-4-(4,4-dimethylpiperidin-1-yl)-2-methylpyridin-3-yl)-2-(tert-butoxy)acetate (0.15 g, 0.329 mmol, 1 equiv), (6-fluoropyridin-3-yl)boronic acid (0.070 g, 0.494 mmol, 1.5 equiv), SPhos (0.027 g, 0.066 mmol, 0.2 equiv), palladium(II) acetate (7.4 mg, 0.033 mmol, 0.1 equiv) and 2 M K₃PO₄ (0.494 ml, 0.988 mmol, 3 equiv) in dioxane (3 mL) was heated at 90° C. for 4 h. Upon cooling to ambient temperature, the reaction mixture was filtered through celite/Na₂SO₄, diluted with ethyl acetate, concentrated in vacuo, and purified by silica gel flash chromatography (0-50% ethyl acetate in hexanes) to afford the product (84 mg, 54%) as a colorless viscous oil. LCMS (M+1): 472.20.

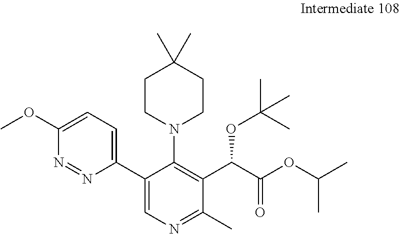 [SMILES: COc1ccc(-c2cnc(C)c([C@H](OC(C)(C)C)C(=O)OC(C)C)c2N2CCC(C)(C)CC2)nn1]

### (S)-Isopropyl 2-(tert-butoxy)-2-(4-(4, 4-dimethylpiperidin-1-yl)-5-(6-methoxypyridazin-3-yl)-2-methylpyridin-3-yl)acetate

The product was prepared according to procedure for the preparation of isopropyl (S)-2-(tert-butoxy)-2-(4-(4,4-dimethylpiperidin-1-yl)-5-(5-(4-fluorophenethoxy)pyrimidin-2-yl)-2-methylpyridin-3-yl)acetate by using (S)-(5-(1-(tert-butoxy)-2-isopropoxy-2-oxoethyl)-4-(4,4-dimethylpiperidin-1-yl)-6-methylpyridin-3-yl)boronic acid (113 mg, 0.269 mmol) and SPhos-Pd-G3 (10.47 mg, 0.013 mmol) was added tribasic potassium phosphate (514 mg, 2.419 mmol) and 3-chloro-6-methoxypyridazine (0.269 mmol) to afford the product (S)-isopropyl 2-(tert-butoxy)-2-(4-(4,4-dimethylpiperidin-1-yl)-5-(6-methoxypyridazin-3-yl)-2-methylpyridin-3-yl)acetate (22 mg, 0.045 mmol, 16.89% yield over 2 steps). LCMS (M+1)=485.3.

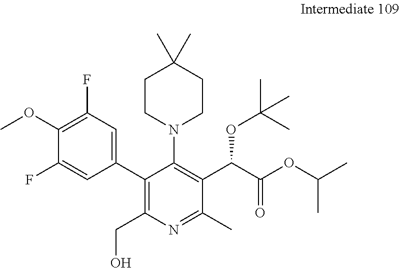 [SMILES: COc1c(F)cc(-c2c(CO)nc(C)c([C@H](OC(C)(C)C)C(=O)OC(C)C)c2N2CCC(C)(C)CC2)cc1F]

### (S)-Isopropyl 2-(tert-butoxy)-2-(5-(3, 5-difluoro-4-methoxyphenyl)-4-(4, 4-dimethylpiperidin-1-yl)-6-(hydroxymethyl)-2-methylpyridin-3-yl)acetate

To a 10 mL Schlenk flask tube equipped with a stir was added tribasic potassium phosphate (590 mg, 2.78 mmol), (3,5-difluoro-4-methoxyphenyl)boronic acid (174 mg, 0.927 mmol), and SPhos-Pd-G3 (24 mg, 0.031 mmol). The flask was sealed with a rubber septum and then placed under N₂ atmosphere. To the flask was added a degassed (N₂ sparging for 5 min) solution of (S)-isopropyl 2-(5-bromo-4-(4,4-dimethylpiperidin-1-yl)-6-(hydroxymethyl)-2-methylpyridin-3-yl)-2-(tert-butoxy)acetate (300 mg, 0.618 mmol) in dioxane (3 mL) and water (1 mL). The flask was placed in a 60° C. oil bath with stirring for 18 h. The reaction mixture was diluted with sat. aq. NaCl (“brine”, 6 mL) and Et₂O (15 mL) and was transferred to a 24 mL test tube. The organic phase was isolated and then dried over MgSO₄, then filtered, then concentrated in vacuo. The resulting residue was dissolved in a min. of acetone and then concentrated onto Celite in vacuo. The resulting powder was subjected to SiO₂ purification (40 g SiO₂ column, hexanes:EtOAc 100:0→60:40) to afford (S)-isopropyl 2-(tert-butoxy)-2-(5-(3,5-difluoro-4-methoxyphenyl)-4-(4,4-dimethylpiperidin-1-yl)-6-(hydroxymethyl)-2-methylpyridin-3-yl)acetate as a white solid foam (223 mg, 66%). ¹H NMR (500 MHz, CDCl₃) δ 7.30 (dd, J=5.2, 1.7 Hz, 1H), 6.87-6.80 (m, 1H), 6.78-6.71 (m, 1H), 5.95 (br s, 1H), 5.14-5.05 (m, 1H), 4.44 (d, J=15.3 Hz, 1H), 4.12 (d, J=15.3 Hz, 1H), 4.09 (s, 3H), 3.35-3.11 (m, 1H), 2.98-2.73 (m, 1H), 2.64 (s, 3H), 2.47-2.27 (m, 1H), 2.24-2.00 (m, 1H), 1.26-1.24 (m, 4H), 1.22 (d, J=6.3 Hz, 4H), 1.18 (s, 11H), 0.98-0.84 (m, 4H), 0.81-0.65 (m, 3H).

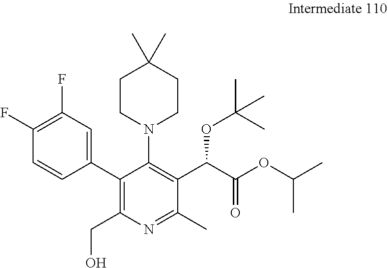 [SMILES: Cc1nc(CO)c(-c2ccc(F)c(F)c2)c(N2CCC(C)(C)CC2)c1[C@H](OC(C)(C)C)C(=O)OC(C)C]

### (S)-Isopropyl 2-(tert-butoxy)-2-(5-(3, 4-difluorophenyl)-4-(4, 4-dimethylpiperidin-1-yl)-6-(hydroxymethyl)-2-methylpyridin-3-yl)acetate

To a 14 mL test tube equipped with a stir was added tribasic potassium phosphate (197 mg, 0.927 mmol), (3,4-difluorophenyl)boronic acid (48.8 mg, 0.309 mmol), (S)-isopropyl 2-(5-bromo-4-(4,4-dimethylpiperidin-1-yl)-6-(hydroxymethyl)-2-methylpyridin-3-yl)-2-(tert-butoxy)acetate (100 mg, 0.206 mmol), and SPhos-Pd-G3 (8 mg, 10 μmol). The test tube was sealed with a rubber septum and then placed under N₂ atmosphere. To the test tube was added a degassed (5 min. N₂ sparging) solution of dioxane (0.75 mL) and water (0.25 mL). The test tube was placed in a 60° C. heating block with for 18 h. The reaction mixture was cooled to r.t., then was diluted with brine (2 mL) and Et₂O (5 mL). The isolated organic phase was dried over MgSO₄, then filtered, then concentrated in vacuo. The resulting residue was dissolved in a min. of acetone and then was concentrated onto Celite in vacuo. The resulting powder was subjected to SiO₂ purification (24 g SiO₂ column, hexanes:EtOAc 100:0→hexanes:EtOAc 60:40) to afford (S)-isopropyl 2-(tert-butoxy)-2-(5-(3,4-difluorophenyl)-4-(4,4-dimethylpiperidin-1-yl)-6-(hydroxymethyl)-2-methylpyridin-3-yl)acetate as a colorless solid foam (61.5 mg, 58%).

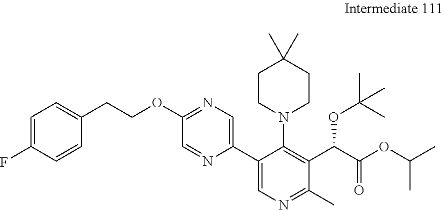 [SMILES: Cc1ncc(-c2cnc(OCCc3ccc(F)cc3)cn2)c(N2CCC(C)(C)CC2)c1[C@H](OC(C)(C)C)C(=O)OC(C)C]

### (S)-Isopropyl 2-(tert-butoxy)-2-(4-(4, 4-dimethylpiperidin-1-yl)-5-(5-(4-fluorophenethoxy)pyrazin-2-yl)-2-methylpyridin-3-yl)acetate

The product was prepared according to procedure for the preparation of isopropyl (S)-2-(tert-butoxy)-2-(4-(4,4-dimethylpiperidin-1-yl)-5-(5-(4-fluorophenethoxy)pyrimidin-2-yl)-2-methylpyridin-3-yl)acetate by using (S)-(5-(1-(tert-butoxy)-2-isopropoxy-2-oxoethyl)-4-(4,4-dimethylpiperidin-1-yl)-6-methylpyridin-3-yl)boronic acid (113 mg, 0.269 mmol) and SPhos-Pd-G3 (10.47 mg, 0.013 mmol) was added tribasic potassium phosphate (514 mg, 2.419 mmol) and 3-chloro-6-methoxypyridazine (0.269 mmol) to afford the product (S)-isopropyl 2-(tert-butoxy)-2-(4-(4,4-dimethylpiperidin-1-yl)-5-(5-(4-fluorophenethoxy)pyrazin-2-yl)-2-methylpyridin-3-yl)acetate (30 mg, 0.051 mmol, 18.83% yield). LCMS (M+1)=593.3.

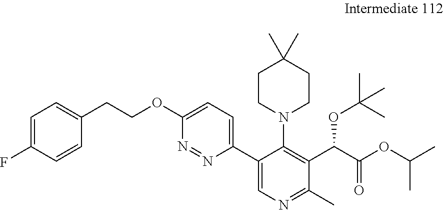 [SMILES: Cc1ncc(-c2ccc(OCCc3ccc(F)cc3)nn2)c(N2CCC(C)(C)CC2)c1[C@H](OC(C)(C)C)C(=O)OC(C)C]

### (S)-Isopropyl 2-(tert-butoxy)-2-(4-(4, 4-dimethylpiperidin-1-yl)-5-(6-(4-fluorophenethoxy)pyridazin-3-yl)-2-methylpyridin-3-yl)acetate

The product was prepared according to procedure for the preparation of isopropyl (S)-2-(tert-butoxy)-2-(4-(4,4-dimethylpiperidin-1-yl)-5-(5-(4-fluorophenethoxy)pyrimidin-2-yl)-2-methylpyridin-3-yl)acetate by using (S)-(5-(1-(tert-butoxy)-2-isopropoxy-2-oxoethyl)-4-(4,4-dimethylpiperidin-1-yl)-6-methylpyridin-3-yl)boronic acid (113 mg, 0.269 mmol) and SPhos-Pd-G3 (10.47 mg, 0.013 mmol) was added tribasic potassium phosphate (514 mg, 2.419 mmol) and 3-chloro-6-(4-fluorophenethoxy)pyridazine (0.269 mmol) to afford the product (S)-isopropyl 2-(tert-butoxy)-2-(4-(4,4-dimethylpiperidin-1-yl)-5-(6-(4-fluorophenethoxy)pyridazin-3-yl)-2-methylpyridin-3-yl)acetate (47 mg, 0.079 mmol, 29.5% yield). LCMS (M+1)=593.3.

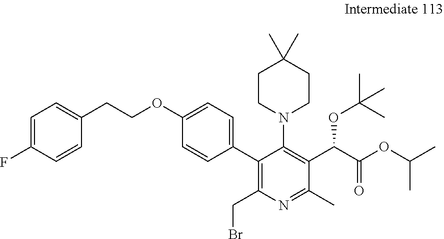 [SMILES: Cc1nc(CBr)c(-c2ccc(OCCc3ccc(F)cc3)cc2)c(N2CCC(C)(C)CC2)c1[C@H](OC(C)(C)C)C(=O)OC(C)C]

### (S)-Isopropyl 2-(6-(bromomethyl)-4-(4, 4-dimethylpiperidin-1-yl)-5-(4-(4-fluorophenethoxy)phenyl)-2-methylpyridin-3-yl)-2-(tert-butoxy)acetate

To a solution of (S)-isopropyl 2-(tert-butoxy)-2-(4-(4,4-dimethylpiperidin-1-yl)-5-(4-(4-fluorophenethoxy)phenyl)-6-(hydroxymethyl)-2-methylpyridin-3-yl)acetate (930 mg, 1.498 mmol) in CH₂Cl₂ (20 mL) was added CBr₄ (546 mg, 1.648 mmol) followed by Ph₃P (432 mg, 1.648 mmol) and the resulting mixture was stirred at room temp for 16 h. Water (10 mL) was then added and the mixture was extracted with dichloromethane (10 mL), dried (Na₂SO₄), filtered and concentrated. The residue was then purified by Biotage (5-30% EtOAc/hexane) to afford(S)-isopropyl 2-(6-(bromomethyl)-4-(4,4-dimethylpiperidin-1-yl)-5-(4-(4-fluorophenethoxy)phenyl)-2-methylpyridin-3-yl)-2-(tert-butoxy)acetate (850 mg, 1.243 mmol, 83% yield) as white solid. ¹H NMR (500 MHz, CDCl₃) δ 7.38 (d, J=8.2 Hz, 1H), 7.31 (br. s., 1H), 7.13 (d, J=7.7 Hz, 2H), 7.05 (t, J=8.4 Hz, 2H), 6.99 (t, J=7.2 Hz, 2H), 6.07 (br. s., 1H), 5.11 (dt, J=12.5, 6.3 Hz, 1H), 4.34 (d, J=9.3 Hz, 1H), 4.25 (br. s., 2H), 4.18 (d, J=9.3 Hz, 1H), 3.20 (d, J=11.7 Hz, 1H), 3.14 (t, J=6.9 Hz, 2H), 2.87 (t, J=12.7 Hz, 1H), 2.63 (s, 3H), 2.30 (d, J=9.6 Hz, 1H), 2.11-1.96 (m, 1H), 1.51 (br. s., 1H), 1.37 (br. s., 1H), 1.24 (d, J=6.3 Hz, 3H), 1.26 (d, J=6.5 Hz, 3H), 1.20 (s, 9H), 1.09 (d, J=14.5 Hz, 1H), 0.91 (br. s., 3H), 0.66 (br. s., 3H). LCMS (M+2H)=685.4.

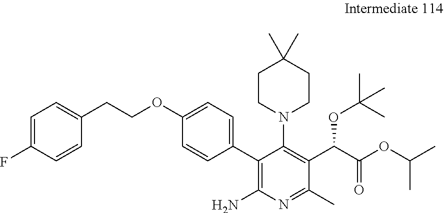 [SMILES: Cc1nc(N)c(-c2ccc(OCCc3ccc(F)cc3)cc2)c(N2CCC(C)(C)CC2)c1[C@H](OC(C)(C)C)C(=O)OC(C)C]

### Isopropyl (S)-2-(6-amino-4-(4, 4-dimethylpiperidin-1-yl)-5-(4-(4-fluorophenethoxy)phenyl)-2-methylpyridin-3-yl)-2-(tert-butoxy)acetate

A mixture of (S)-isopropyl 2-(6-amino-5-bromo-4-(4,4-dimethylpiperidin-1-yl)-2-methylpyridin-3-yl)-2-(tert-butoxy)acetate (1.7 g, 3.61 mmol), (4-(4-fluorophenethoxy)phenyl)boronic acid (1.880 g, 7.23 mmol), 2-dicyclohexylphosphino-2′,6′-dimethoxybiphenyl (0.297 g, 0.723 mmol), palladium(II) acetate (0.081 g, 0.361 mmol) and 2 M K₃PO₄ (5.42 ml, 10.84 mmol) in dioxane (36 mL) was heated at 80° C. for 1 h. After cooling to ambient temperature, the reaction mixture was filtered through celite, diluted with ethyl acetate, washed with brine, dried (Na₂SO₄), and concentrated in vacuo. The crude product was purified on silica gel (220 g column) using 5-80% ethyl acetate in hexanes to give a light orange solid (1.55 g, 71%). LCMS (M+1)=606.35.

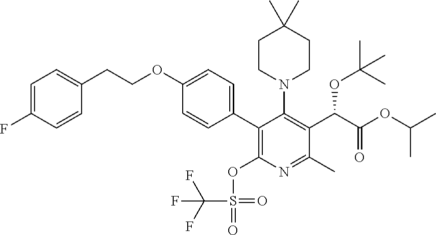 [SMILES: Cc1nc(OS(=O)(=O)C(F)(F)F)c(-c2ccc(OCCc3ccc(F)cc3)cc2)c(N2CCC(C)(C)CC2)c1[C@H](OC(C)(C)C)C(=O)OC(C)C]

### (S)-Isopropyl 2-(tert-butoxy)-2-(4-(4, 4-dimethylpiperidin-1-yl)-5-(4-(4-fluorophenethoxy)phenyl)-2-methyl-6-(((trifluoromethyl)sulfonyl)oxy)pyridin-3-yl)acetate

(S)-Isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.6 g, 0.842 mmol) and sodium nitrite (0.145 g, 2.105 mmol) were mixed together and added in portions to a mixture of hexanes (2.1 mL), DMSO (0.21 mL) and trifluoromethanesulfonic acid (0.229 ml, 2.53 mmol) at 5° C. The mixture was stirred for 10 min at 5° C., then stirred at ambient temperature overnight. Water was added to the reaction mixture and it was extracted with DCM, dried (Na₂SO₄) and concentrated in vacuo. The residue was purified on silica gel (80 g column) using 0-40% ethyl acetate in hexanes, then 40-100% ethyl acetate in hexanes. The desired fractions were concentrated in vacuo give desired compound as pale yellow glass (0.25 g, 40%). LCMS (M+1)=739.3.

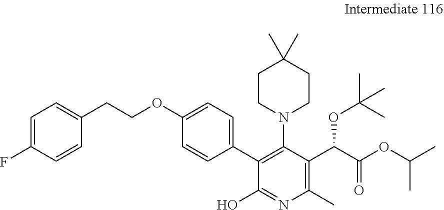 [SMILES: Cc1nc(O)c(-c2ccc(OCCc3ccc(F)cc3)cc2)c(N2CCC(C)(C)CC2)c1[C@H](OC(C)(C)C)C(=O)OC(C)C]

### (S)-Isopropyl 2-(tert-butoxy)-2-(4-(4, 4-dimethylpiperidin-1-yl)-5-(4-(4-fluorophenethoxy)phenyl)-6-hydroxy-2-methylpyridin-3-yl)acetate

To a solution of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.21 g, 0.350 mmol, 1 equiv) in AcOH (3.5 mL) was added NaNO₂ (0.15 g, 2.10 mmol, 6 equiv) portionwise. After 1 h, the orange solution was concentrated under a stream of nitrogen. The crude intermediate was taken up in MeOH (3.5 mL) and THF (1 mL). Potassium carbonate (0.39 g, 2.80 mmol, 8 equiv) was added. After 1 h, the reaction was added to 10% citric acid solution and extracted with DCM (×3). The combined DCM layers were dried (Na₂SO₄) and concentrated in vacuo. The crude product was purified by silica gel flash column chromatography (50-100% EtOAc/hex) to provide the product (0.20 g, 93%) as a yellow foam. ¹H NMR (500 MHz, CDCl₃) δ 7.28-7.24 (m, 3H), 7.07-6.91 (m, 5H), 5.58 (s, 1H), 5.09 (dt, J=12.5, 6.2 Hz, 1H), 4.21 (td, J=7.0, 1.3 Hz, 2H), 3.34-3.20 (m, 1H), 3.11 (t, J=6.9 Hz, 2H), 2.83 (br s, 1H), 2.38 (s, 3H), 2.29-2.21 (m, 1H), 2.08 (d, J=8.5 Hz, 1H), 1.42-1.28 (m, 3H), 1.28 (d, J=6.3 Hz, 3H), 1.25 (d, J=6.3 Hz, 3H), 1.21 (s, 9H), 1.16-1.09 (m, 1H), 0.91 (br d, J=3.8 Hz, 3H), 0.73 (br s, 3H). LCMS (M+1)=607.3.

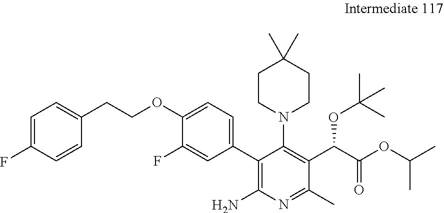 [SMILES: Cc1nc(N)c(-c2ccc(OCCc3ccc(F)cc3)c(F)c2)c(N2CCC(C)(C)CC2)c1[C@H](OC(C)(C)C)C(=O)OC(C)C]

### Isopropyl (S)-2-(6-amino-4-(4, 4-dimethylpiperidin-1-yl)-5-(3-fluoro-4-(4-fluorophenethoxy)phenyl)-2-methylpyridin-3-yl)-2-(tert-butoxy)acetate

A mixture of 2-(3-fluoro-4-(4-fluorophenethoxy)phenyl)-6-methyl-1,3,6,2-dioxazaborocane-4,8-dione (0.27 g, 0.701 mmol), (S)-isopropyl 2-(6-amino-5-bromo-4-(4,4-dimethylpiperidin-1-yl)-2-methylpyridin-3-yl)-2-(tert-butoxy)acetate (0.22 g, 0.468 mmol), 2-dicyclohexylphosphino-2′,6′-dimethoxybiphenyl (0.058 g, 0.140 mmol), palladium(II) acetate (0.016 g, 0.070 mmol) and 2 M K₃PO₄ (2.81 ml, 5.61 mmol) in dioxane (5 mL) was heated at 80° C. for 2 h. The reaction mixture was filtered through celite, diluted with ethyl acetate, washed with brine, dried (Na₂SO₄), and concentrated in vacuo. The crude product was purified on silica gel (80 g column) using 0-70% ethyl acetate in hexanes to give an orange solid (0.29 g, 99%). LCMS (M+1)=624.45.

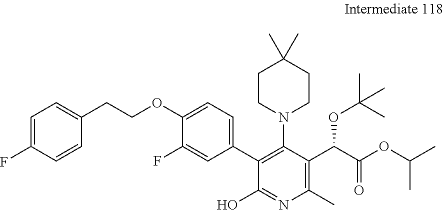 [SMILES: Cc1nc(O)c(-c2ccc(OCCc3ccc(F)cc3)c(F)c2)c(N2CCC(C)(C)CC2)c1[C@H](OC(C)(C)C)C(=O)OC(C)C]

### (S)-Isopropyl 2-(tert-butoxy)-2-(4-(4, 4-dimethylpiperidin-1-yl)-5-(3-fluoro-4-(4-fluorophenethoxy)phenyl)-6-hydroxy-2-methylpyridin-3-yl)acetate

Sodium nitrite (0.119 g, 1.731 mmol) was added to a solution of (S)-isopropyl 2-(6-amino-4-(4,4-dimethylpiperidin-1-yl)-5-(3-fluoro-4-(4-fluorophenethoxy)phenyl)-2-methylpyridin-3-yl)-2-(tert-butoxy)acetate (0.18 g, 0.289 mmol) in acetic acid (2.9 mL). The mixture was stirred at ambient temperature for 1 h and concentrated. The residue was taken up in methanol (5 mL) and THF (1 mL). K₂CO₃ (0.399 g, 2.89 mmol) was added and the mixture was stirred at ambient temperature for 1 h. 10% citric acid was added and the mixture was extracted with DCM (×3). The combined organic extracts were dried (Na₂SO₄) and concentrated in vacuo. The residue was purified on silica (40 g column) using 40-100% ethyl acetate in hexanes to give a brown solid (0.15 g, 83%). LCMS (M+1)=625.35.

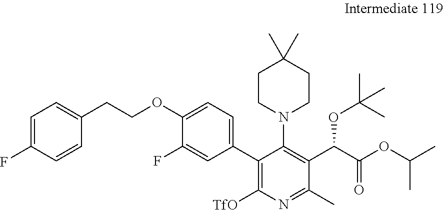 [SMILES: Cc1nc(OS(=O)(=O)C(F)(F)F)c(-c2ccc(OCCc3ccc(F)cc3)c(F)c2)c(N2CCC(C)(C)CC2)c1[C@H](OC(C)(C)C)C(=O)OC(C)C]

### Isopropyl (S)-2-(tert-butoxy)-2-(4-(4, 4-dimethylpiperidin-1-yl)-5-(3-fluoro-4-(4-fluorophenethoxy)phenyl)-2-methyl-6-(((trifluoromethyl)sulfonyl)oxy)pyridin-3-yl)acetate

To a solution of (S)-isopropyl 2-(tert-butoxy)-2-(4-(4,4-dimethylpiperidin-1-yl)-5-(3-fluoro-4-(4-fluorophenethoxy)phenyl)-6-hydroxy-2-methylpyridin-3-yl)acetate (0.07 g, 0.112 mmol) and pyridine (0.091 ml, 1.120 mmol) in DCM (3 mL) at 0° C. was added triflic anhydride (0.026 ml, 0.153 mmol). The mixture was stirred at 0° C. for 3 h and then quenched with saturated aqueous NaHCO₃. The mixture was extracted with ethyl acetate. The organic layer was washed with brine, dried (Na₂SO₄), and concentrated in vacuo. The residue was purified on silica (40 g column) using 0-40% ethyl acetate in hexanes to give a white foamy solid (55 mg, 65%). LCMS (M+1)=757.25.

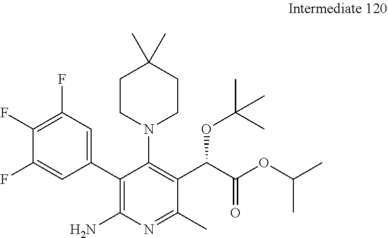 [SMILES: Cc1nc(N)c(-c2cc(F)c(F)c(F)c2)c(N2CCC(C)(C)CC2)c1[C@H](OC(C)(C)C)C(=O)OC(C)C]

### (S)-Isopropyl 2-(6-amino-4-(4, 4-dimethylpiperidin-1-yl)-2-methyl-5-(3,4, 5-trifluorophenyl)pyridin-3-yl)-2-(tert-butoxy)acetate

A mixture of (S)-isopropyl 2-(6-amino-5-bromo-4-(4,4-dimethylpiperidin-1-yl)-2-methylpyridin-3-yl)-2-(tert-butoxy)acetate (0.2 g, 0.425 mmol), (3,4,5-trifluorophenyl)boronic acid (0.075 g, 0.425 mmol), 2-dicyclohexylphosphino-2′,6′-dimethoxybiphenyl (0.013 g, 0.033 mmol), palladium(II) acetate (9.54 mg, 0.043 mmol), and 2 M K₃PO₄ (0.850 mL, 1.701 mmol) in dioxane (4.25 mL) was degassed and heated at 100° C. for 2 h in a microwave. The reaction mixture was then diluted with ethyl acetate, washed with brine, dried (Na₂SO₄), and concentrated in vacuo. The residue was purified by silica gel column chromatography (10-100% ethyl acetate in hexanes). The desired fractions were concentrated in vacuo to give the product as a white solid (0.19 g, 86%). ¹H NMR (500 MHz, CDCl₃) δ 7.12-6.99 (m, 1H), 6.95-6.85 (m, 1H), 5.82 (br. s., 1H), 5.09 (dt, J=12.5, 6.3 Hz, 1H), 4.18 (s, 2H), 3.32 (d, J=12.0 Hz, 1H), 2.92-2.74 (m, 1H), 2.48 (s, 3H), 2.36 (d, J=10.9 Hz, 1H), 2.14-1.98 (m, 1H), 1.57-1.54 (m, 2H), 1.45-1.37 (m, 2H), 1.26 (d, J=6.3 Hz, 3H), 1.24 (d, J=6.3 Hz, 3H), 1.20 (s, 9H), 0.94 (s, 3H), 0.74 (s, 3H). LCMS (M+1)=522.40.

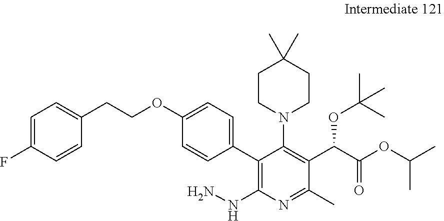 [SMILES: Cc1nc(NN)c(-c2ccc(OCCc3ccc(F)cc3)cc2)c(N2CCC(C)(C)CC2)c1[C@H](OC(C)(C)C)C(=O)OC(C)C]

### (S)-Isopropyl 2-(tert-butoxy)-2-(4-(4, 4-dimethylpiperidin-1-yl)-5-(4-(4-fluorophenethoxy)phenyl)-6-hydrazinyl-2-methylpyridin-3-yl)acetate

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.075 g, 0.102 mmol) and hydrazine (0.325 g, 10.15 mmol) in toluene (5.1 mL) was heated at 150° C. for 1 h in the microwave. The reaction mixture was diluted with ethyl acetate, washed with brine, dried (Na₂SO₄), and concentrated in vacuo give a pale yellow foam which was used as is for further reactions. LCMS (M+1)=621.25.

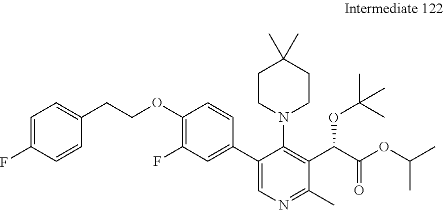 [SMILES: Cc1ncc(-c2ccc(OCCc3ccc(F)cc3)c(F)c2)c(N2CCC(C)(C)CC2)c1[C@H](OC(C)(C)C)C(=O)OC(C)C]

### Isopropyl (S)-2-(tert-butoxy)-2-(4-(4, 4-dimethylpiperidin-1-yl)-5-(3-fluoro-4-(4-fluorophenethoxy)phenyl)-2-methylpyridin-3-yl)acetate

To a 14 mL test tube equipped with a stir was added tribasic potassium phosphate (419 mg, 1.98 mmol), 2-(3-fluoro-4-(4-fluorophenethoxy)phenyl)-6-methyl-1,3,6,2-dioxazaborocane-4,8-dione (103 mg, 0.263 mmol), (S)-isopropyl 2-(5-bromo-4-(4,4-dimethylpiperidin-1-yl)-2-methylpyridin-3-yl)-2-(tert-butoxy)acetate (100 mg, 0.220 mmol), and SPhos-Pd-G3 (8.6 mg, 11 μmol). The test tube was sealed with a rubber septum and then placed under nitrogen atmosphere. To the test tube was added a degassed (5 minutes of nitrogen sparging) mixture of dioxane (1.5 mL) and water (0.5 mL). The test tube was placed in a 60° C. heating block with stirring for 18 h. The reaction mixture was transferred to 125 mL separatory funnel and was diluted with Et₂O (25 mL). The mixture was washed with water (25 mL), then dried over MgSO₄; filtered; then concentrated in vacuo. The resulting residue was dissolved in a minimum of acetone, then was concentrated onto Celite in vacuo. The resulting powder was subjected to SiO₂ purification (25 g SiO₂ column, hexanes:EtOAc 100:0→50:50) to afford a solid residue, isopropyl (S)-2-(tert-butoxy)-2-(4-(4,4-dimethylpiperidin-1-yl)-5-(3-fluoro-4-(4-fluorophenethoxy)phenyl)-2-methylpyridin-3-yl)acetate. ESI-MS(+) m/z=609.3 (M+1).

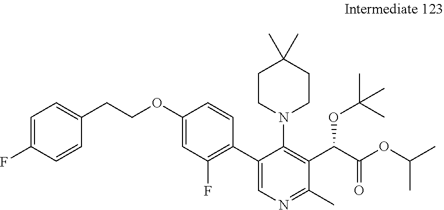 [SMILES: Cc1ncc(-c2ccc(OCCc3ccc(F)cc3)cc2F)c(N2CCC(C)(C)CC2)c1[C@H](OC(C)(C)C)C(=O)OC(C)C]

### Isopropyl (S)-2-(tert-butoxy)-2-(4-(4, 4-dimethylpiperidin-1-yl)-5-(2-fluoro-4-(4-fluorophenethoxy)phenyl)-2-methylpyridin-3-yl)acetate

To a 14 mL test tube equipped with a stir was added tribasic potassium phosphate (419 mg, 1.98 mmol), 2-(2-fluoro-4-(4-fluorophenethoxy)phenyl)-6-methyl-1,3,6,2-dioxazaborocane-4,8-dione (103 mg, 0.263 mmol), (S)-isopropyl 2-(5-bromo-4-(4,4-dimethylpiperidin-1-yl)-2-methylpyridin-3-yl)-2-(tert-butoxy)acetate (100 mg, 0.220 mmol), and SPhos-Pd-G3 (8.6 mg, 11 μmol). The test tube was sealed with a rubber septum and then placed under N₂ atm. To the flask was added a degassed (5 min N₂ sparging) mixture of dioxane (1.5 mL) and water (0.5 mL). The test tube was placed in a 60° C. heating block with stirring for 18 h. The reaction mixture was transferred to 125 mL separatory funnel and was diluted with Et₂O (25 mL). The mixture was washed with water (25 mL), then dried over MgSO₄; filtered; then concentrated in vacuo. The resulting residue was dissolved in a min. of acetone, then was concentrated onto Celite in vacuo. The resulting powder was subjected to SiO₂ purification (24 g SiO₂ column, hexanes:EtOAc 100:0→50:50) to afford isopropyl (S)-2-(tert-butoxy)-2-(4-(4,4-dimethylpiperidin-1-yl)-5-(2-fluoro-4-(4-fluorophenethoxy)phenyl)-2-methylpyridin-3-yl)acetate as a solid. ESI-MS(+) m/z=609.3 (M+1).

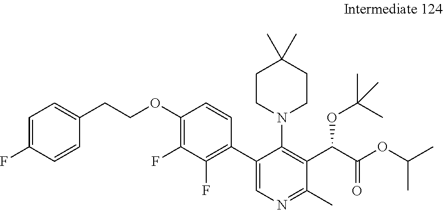 [SMILES: Cc1ncc(-c2ccc(OCCc3ccc(F)cc3)c(F)c2F)c(N2CCC(C)(C)CC2)c1[C@H](OC(C)(C)C)C(=O)OC(C)C]

### (S)-Isopropyl 2-(tert-butoxy)-2-(5-(2, 3-difluoro-4-(4-fluorophenethoxy)phenyl)-4-(4, 4-dimethylpiperidin-1-yl)-2-methylpyridin-3-yl)acetate

To a 14 mL test tube equipped with a stir was added tribasic potassium phosphate (419 mg, 1.97 mmol), 2-(2,3-difluoro-4-(4-fluorophenethoxy)phenyl)-6-methyl-1,3,6,2-dioxazaborocane-4,8-dione (107 mg, 0.263 mmol), (S)-isopropyl 2-(5-bromo-4-(4,4-dimethylpiperidin-1-yl)-2-methylpyridin-3-yl)-2-(tert-butoxy)acetate (100 mg, 0.220 mmol), and SPhos-Pd-G3 (8.6 mg, 11 μmol). The test tube was sealed with a rubber septum and then placed under N₂ atm. To the flask was added a degassed (N₂ sparging for 5 min.) solution of dioxane (1.5 mL) and water (0.5 mL). The test tube was placed in a 60° C. heating block with stirring for 18 h. The reaction mixture was transferred to 125 mL separatory funnel and was diluted with Et₂O (25 mL). The mixture was washed with water (25 mL), then dried over MgSO₄; filtered; then concentrated in vacuo. The resulting residue was dissolved in a min. of acetone, then was concentrated onto Celite in vacuo. The resulting powder was subjected to SiO₂ purification (24 g SiO₂ column, hexanes:EtOAc 100:0→50:50) to afford (S)-isopropyl 2-(tert-butoxy)-2-(5-(2,3-difluoro-4-(4-fluorophenethoxy)phenyl)-4-(4,4-dimethylpiperidin-1-yl)-2-methylpyridin-3-yl)acetate as a solid. ESI-MS(+) m/z=627.3 (M+1).

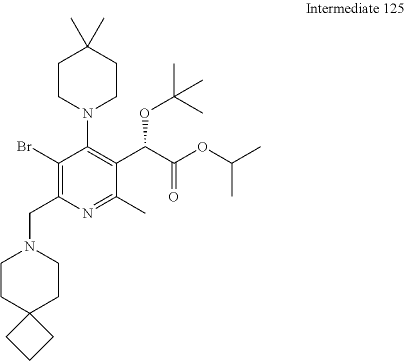 [SMILES: Cc1nc(CN2CCC3(CCC3)CC2)c(Br)c(N2CCC(C)(C)CC2)c1[C@H](OC(C)(C)C)C(=O)OC(C)C]

### (S)-Isopropyl 2-(6-(7-azaspiro[3.5]nonan-7-ylmethyl)-5-bromo-4-(4, 4-dimethylpiperidin-1-yl)-2-methylpyridin-3-yl)-2-(tert-butoxy)acetate

To a 25 mL r.b. flask equipped with a stir bar and charged with (S)-isopropyl 2-(5-bromo-4-(4,4-dimethylpiperidin-1-yl)-6-formyl-2-methylpyridin-3-yl)-2-(tert-butoxy)acetate (644.7 mg, 1.33 mmol) was added 7-azaspiro[3.5]nonane (334 mg, 2.67 mmol) and DCM (10 mL). To the solution was added acetic acid (0.191 mL, 3.33 mmol). To the solution was added sodium triacetoxyborohydride (565 mg, 2.67 mmol) and MeOH (5 mL) to afford a homogeneous orange solution. The solution was stirred at r.t. for 18 h. The reaction solution was concentrated in vacuo and the resulting residue was dissolved in EtOAc (25 mL), then transferred to a 125 mL separatory funnel. The solution was washed with aq. NaOH (1M, 25 mL), then brine (15 mL). The organic phase was dried over MgSO₄, filtered and concentrated in vacuo. The resulting residue was dissolved in a min. of acetone, then was concentrated onto Celite in vacuo. The resulting powder was subjected to SiO₂ purification (40 g column, hexanes:EtOAc 100:0→70:30) to afford (S)-isopropyl 2-(6-(7-azaspiro[3.5]nonan-7-ylmethyl)-5-bromo-4-(4,4-dimethylpiperidin-1-yl)-2-methylpyridin-3-yl)-2-(tert-butoxy)acetate as a solid foam (577 mg, 73%). ¹H NMR (500 MHz, CDCl₃) δ 6.25 (br s, 1H), 5.03 (quin, J=6.2 Hz, 1H), 4.04 (br s, 1H), 3.78-3.63 (m, 2H), 3.57-3.45 (m, 1H), 2.88 (br d, J=11.3 Hz, 1H), 2.62 (br d, J=11.8 Hz, 1H), 2.56 (s, 3H), 2.49 (br s, 4H), 1.90-1.80 (m, 2H), 1.76-1.70 (m, 4H), 1.70-1.66 (m, 1H), 1.62 (brt, J=5.4 Hz, 4H), 1.60-1.52 (m, 2H), 1.43 (br d, J=12.1 Hz, 1H), 1.37-1.30 (m, 1H), 1.21-1.17 (m, 9H), 1.19 (d, J=6.1 Hz, 3H), 1.11 (d, J=6.1 Hz, 3H), 1.07 (s, 3H), 1.02 (s, 3H).

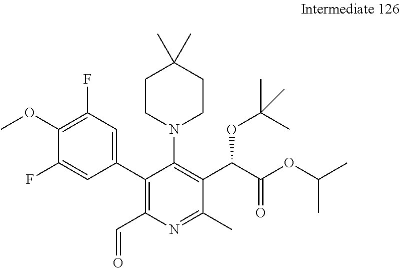 [SMILES: COc1c(F)cc(-c2c(C=O)nc(C)c([C@H](OC(C)(C)C)C(=O)OC(C)C)c2N2CCC(C)(C)CC2)cc1F]

### (S)-Isopropyl 2-(tert-butoxy)-2-(5-(3, 5-difluoro-4-methoxyphenyl)-4-(4, 4-dimethylpiperidin-1-yl)-6-formyl-2-methylpyridin-3-yl)acetate

To a 25 mL r.b. flask equipped with a stir bar and charged with (S)-isopropyl 2-(tert-butoxy)-2-(5-(3,5-difluoro-4-methoxyphenyl)-4-(4,4-dimethylpiperidin-1-yl)-6-(hydroxymethyl)-2-methylpyridin-3-yl)acetate (213 mg, 0.388 mmol) in DCM (3.5 mL) and MeCN (0.5 mL) was added Dess-Martin periodinane (247 mg, 0.582 mmol). The solution was stirred at r.t. for 7 h. The reaction mixture was transferred to a 125 mL separatory funnel and was diluted with Et₂O (25 mL). The solution was washed with aq. NaOH (1M, 15 mL). The aq. phase was extracted with Et₂O (25 mL). The combined organics were washed with brine (15 mL), then dried over MgSO₄, then filtered, then concentrated in vacuo. The resulting residue was dissolved in a min. of acetone, the was concentrated onto Celite in vacuo. The resulting powder was subjected to SiO₂ purification (24 g column, hexanes:EtOAc 100:0480:20) to afford (S)-isopropyl 2-(tert-butoxy)-2-(5-(3,5-difluoro-4-methoxyphenyl)-4-(4,4-dimethylpiperidin-1-yl)-6-formyl-2-methylpyridin-3-yl)acetate as a white solid foam (172.3 mg, 81%). ¹H NMR (500 MHz, CDCl₃) δ 9.90 (s, 1H), 6.89 (br d, J=10.7 Hz, 1H), 6.83 (br d, J=10.7 Hz, 1H), 6.03 (br s, 1H), 5.11 (dt, J=12.5, 6.3 Hz, 1H), 4.10 (s, 3H), 3.26-3.18 (m, 1H), 2.99-2.89 (m, 1H), 2.71 (s, 3H), 2.43-2.33 (m, 1H), 2.17-2.09 (m, 1H), 1.46-1.26 (m, 4H), 1.25 (d, J=6.3 Hz, 3H), 1.23 (d, J=6.1 Hz, 3H), 1.18 (s, 9H), 0.94 (br s, 3H), 0.74 (br s, 3H).

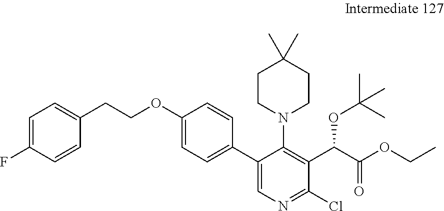 [SMILES: CCOC(=O)[C@@H](OC(C)(C)C)c1c(Cl)ncc(-c2ccc(OCCc3ccc(F)cc3)cc2)c1N1CCC(C)(C)CC1]

### Ethyl-(S)-2-(tert-butoxy)-2-(2-chloro-4-(4,4-dimethylpiperidin-1-yl)-5-(4-(4-fluorophenethoxy)phenyl)pyridin-3-yl)acetate

To a dry reaction vial under nitrogen was added ethyl-(S)-2-(5-bromo-2-chloro-4-(4,4-dimethylpiperidin-1-yl)pyridin-3-yl)-2-(tert-butoxy)acetate (230 mg, 0.498 mmol), (4-(4-fluorophenethoxy)phenyl)boronic acid (190 mg, 0.731 mmol) and THF (25 mL). The reaction was flushed with argon, treated with 0.5 M potassium phosphate tribasic (3 mL, 1.500 mmol), followed 2^{nd} generation X-phos precatalyst (30 mg, 0.038 mmol), capped and stirred at room temp for 18 h. The crude reaction was dissolved in EtOAc, extracted and purified via silica gel chromatography (40 g SiO₂ column, hexane:EtOAc 100:0->0:100) to afford ethyl (S)-2-(tert-butoxy)-2-(2-chloro-4-(4,4-dimethylpiperidin-1-yl)-5-(4-(4-fluorophenethoxy)phenyl)pyridin-3-yl)acetate, 260 mg (87%). LCMS (M+1)=597.3, 599.3.

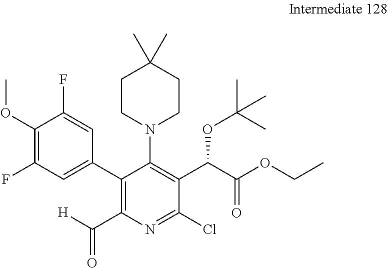 [SMILES: CCOC(=O)[C@@H](OC(C)(C)C)c1c(Cl)nc(C=O)c(-c2cc(F)c(OC)c(F)c2)c1N1CCC(C)(C)CC1]

### Ethyl-(S)-2-(tert-butoxy)-2-(2-chloro-5-(3, 5-difluoro-4-methoxyphenyl)-4-(4, 4-dimethylpiperidin-1-yl)-6-formylpyridin-3-yl)acetate

To a dry pressure vial under nitrogen was added ethyl-(S)-2-(5-bromo-2-chloro-4-(4,4-dimethylpiperidin-1-yl)-6-formylpyridin-3-yl)-2-(tert-butoxy)acetate (180 mg, 0.367 mmol), 3,5-difluoro-4-methoxy-phenylboronic acid (105 mg, 0.559 mmol) and THF (17 mL). The reaction was flushed with argon, treated with 0.5 M potassium phosphate tribasic (2.60 mL, 1.300 mmol), followed by 2^{nd} generation X-phos precatalyst (32 mg, 0.041 mmol), capped and stirred at room temp for 18 h. The crude material was dissolved in EtOAc (200 mL), extracted with water (1×6 mL), brine (1×10 mL), dried over Na₂SO₄, and concentrated. The crude material was purified via silica gel chromatography (40 g SiO₂ column, hexane:EtOAc 100:0->70:30) to afford ethyl (S)-2-(tert-butoxy)-2-(2-chloro-5-(3,5-difluoro-4-methoxyphenyl)-4-(4,4-dimethylpiperidin-1-yl)-6-formylpyridin-3-yl)acetate, 60.2 mg, (30%). LCMS (M+1)=553.3 and 555.3.

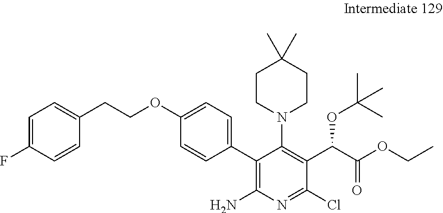 [SMILES: CCOC(=O)[C@@H](OC(C)(C)C)c1c(Cl)nc(N)c(-c2ccc(OCCc3ccc(F)cc3)cc2)c1N1CCC(C)(C)CC1]

### Ethyl (S)-2-(6-amino-2-chloro-4-(4, 4-dimethylpiperidin-1-yl)-5-(4-(4-fluorophenethoxy)phenyl)pyridin-3-yl)-2-(tert-butoxy)acetate

To a dry pressure vial under nitrogen was added (32.2 mg, 0.068 mmol, (4-(4-fluorophenethoxy)phenyl) ethyl-(S)-2-(6-amino-5-bromo-2-chloro-4-(4,4-dimethylpiperidin-1-yl)pyridin-3-yl)-2-(tert-butoxy)acetate boronic acid (27 mg, 0.104 mmol) and THF (4 mL). The reaction was flushed with argon, treated with 0.5 M potassium phosphate tribasic (500 μL, 0.250 mmol) followed by 2^{nd} generation X-phos precatalyst (6.6 mg, 8.39 μmol) and stirred at room temp for 18 h. The reaction was diluted with ethyl acetate (75 mL), extracted with water (1×5 mL), brine (1×5 mL), dried over Na₂SO₄, and concentrated. The crude material was purified via siliga gel chromatography (12 g SiO₂ column, dichloromethane:EtOAc 100:0->0:100) to afford ethyl (S)-2-(6-amino-2-chloro-4-(4,4-dimethylpiperidin-1-yl)-5-(4-(4-fluorophenethoxy)phenyl)pyridin-3-yl)-2-(tert-butoxy)acetate, 34.9 mg (84%). LCMS (M+1)=612.5.

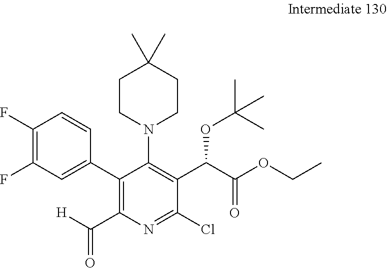 [SMILES: CCOC(=O)[C@@H](OC(C)(C)C)c1c(Cl)nc(C=O)c(-c2ccc(F)c(F)c2)c1N1CCC(C)(C)CC1]

### Ethyl (S)-2-(tert-butoxy)-2-(2-chloro-5-(3, 4-difluorophenyl)-4-(4, 4-dimethylpiperidin-1-yl)-6-formylpyridin-3-yl)acetate

To a reaction vial under nitrogen was added (S)-ethyl 2-(5-bromo-2-chloro-4-(4,4-dimethylpiperidin-1-yl)-6-formylpyridin-3-yl)-2-(tert-butoxy)acetate (97 mg, 0.198 mmol), (3,4-difluorophenyl)boronic acid (60 mg, 0.380 mmol) and THF (8 mL). The reaction was flushed with argon, treated with 0.5 M potassium phosphate tribasic (1.30 mL, 0.650 mmol), followed by 2^{nd} generation X-phos precatalyst (15 mg, 0.019 mmol), capped and stirred at room temp for 18 h. The crude material was dissolved in EtOAc, extracted with brine and purified via silica gel chromatography (12 g SiO₂ column, dichloromethane:EtOAc 100:0->0:100) to afford ethyl (S)-2-(tert-butoxy)-2-(2-chloro-5-(3,4-difluorophenyl)-4-(4,4-dimethylpiperidin-1-yl)-6-formylpyridin-3-yl)acetate, 60.8 mg, (58%). LCMS (M+1)=523.2, 525.2.

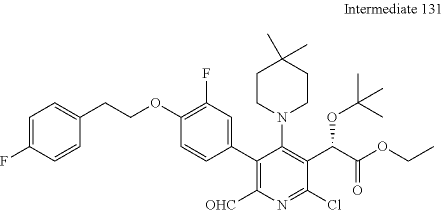 [SMILES: CCOC(=O)[C@@H](OC(C)(C)C)c1c(Cl)nc(C=O)c(-c2ccc(OCCc3ccc(F)cc3)c(F)c2)c1N1CCC(C)(C)CC1]

### Ethyl (S)-2-(tert-butoxy)-2-(2-chloro-4-(4, 4-dimethylpiperidin-1-yl)-5-(3-fluoro-4-(4-fluorophenethoxy)phenyl)-6-formylpyridin-3-yl)acetate

To a dry reaction vial under nitrogen was added ethyl-(S)-2-(5-bromo-2-chloro-4-(4,4-dimethylpiperidin-1-yl)-6-formylpyridin-3-yl)-2-(tert-butoxy)acetate (110.5 mg, 0.226 mmol), 2-(3-fluoro-4-(4-fluorophenethoxy)phenyl)-4,4,5,5-tetramethyl-1,3,2-dioxaborolane (104.7 mg, 0.291 mmol) and THF (9 mL). The reaction was flushed with argon, treated with 0.5 M potassium phosphate tribasic (1.6 mL, 0.800 mmol), followed by 2^{nd} generation X-phos precatalyst (13.6 mg, 0.017 mmol), capped and stirred at room temp for 18 h. The crude reaction was dissolved in EtOAc (110 mL), extracted with water (1×5 mL), brine (1×5 mL), dried over Na₂SO₄ and concentrated. The crude material was purified via silica gel chromatography (24 g SiO₂ column, dichloromethane:EtOAc 100:0->10:90) to afford ethyl (S)-2-(tert-butoxy)-2-(2-chloro-4-(4,4-dimethylpiperidin-1-yl)-5-(3-fluoro-4-(4-fluorophenethoxy)phenyl)-6-formylpyridin-3-yl)acetate, 67.8 mg (47%). LCMS (M+1)=643.3.

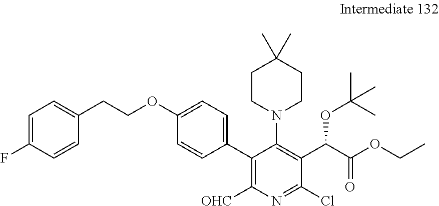 [SMILES: CCOC(=O)[C@@H](OC(C)(C)C)c1c(Cl)nc(C=O)c(-c2ccc(OCCc3ccc(F)cc3)cc2)c1N1CCC(C)(C)CC1]

### Ethyl (S)-2-(tert-butoxy)-2-(2-chloro-4-(4, 4-dimethylpiperidin-1-yl)-5-(4-(4-fluorophenethoxy)phenyl)-6-formylpyridin-3-yl)acetate

To a dry reaction vial under argon was added ethyl-(S)-2-(5-bromo-2-chloro-4-(4,4-dimethylpiperidin-1-yl)-6-formylpyridin-3-yl)-2-(tert-butoxy)acetate (117.5 mg, 0.240 mmol), (4-(4-fluorophenethoxy)phenyl)boronic acid (95 mg, 0.365 mmol) and THF (9 mL). The reaction was flushed with argon, treated with 0.5 M potassium phosphate tribasic (1.65 mL, 0.825 mmol), followed by 2^{nd} generation X-phos precatalyst (15 mg, 0.019 mmol), capped and stirred at room temp for 18 h. The crude material was dissolved in EtOAc, extracted and purified via silica gel chromatography (40 g SiO₂ column, hexane:EtOAc 100:0->0:100) to afford ethyl (S)-2-(tert-butoxy)-2-(2-chloro-4-(4,4-dimethylpiperidin-1-yl)-5-(4-(4-fluorophenethoxy)phenyl)-6-formylpyridin-3-yl)acetate, 80.0 mg (53%). LCMS (M+1)=625.4.

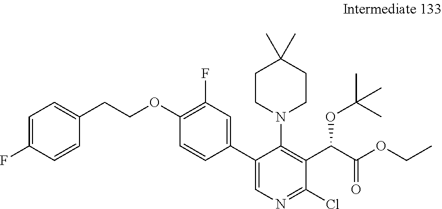 [SMILES: CCOC(=O)[C@@H](OC(C)(C)C)c1c(Cl)ncc(-c2ccc(OCCc3ccc(F)cc3)c(F)c2)c1N1CCC(C)(C)CC1]

### Ethyl (S)-2-(tert-butoxy)-2-(2-chloro-4-(4, 4-dimethylpiperidin-1-yl)-5-(3-fluoro-4-(4-fluorophenethoxy)phenyl)pyridin-3-yl)acetate

To a dry reaction vial under argon was added (S)-ethyl 2-(5-bromo-2-chloro-4-(4,4-dimethylpiperidin-1-yl)pyridin-3-yl)-2-(tert-butoxy)acetate (105 mg, 0.227 mmol), 2-(3-fluoro-4-(4-fluorophenethoxy)phenyl)-4,4,5,5-tetramethyl-1,3,2-dioxaborolane (150 mg, 0.416 mmol) and THF (9 mL). The reaction was flushed with argon, treated with 0.5 M potassium phosphate tribasic (1.70 mL, 0.850 mmol), followed by 2^{nd} generation X-phos precatalyst (9.7 mg, 0.012 mmol), capped and stirred at room temp for 18 h. The reaction was diluted with ethyl acetate, extracted and the crude material was purified via silica gel chromatography (24 g SiO₂ column, dichloromethane:EtOAc 100:0->0:100) to afford ethyl (S)-2-(tert-butoxy)-2-(2-chloro-4-(4,4-dimethylpiperidin-1-yl)-5-(3-fluoro-4-(4-fluorophenethoxy)phenyl)pyridin-3-yl)acetate, 143 mg (82%). LCMS (M+1)=615.3, 617.3.

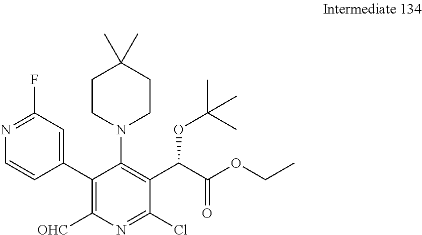 [SMILES: CCOC(=O)[C@@H](OC(C)(C)C)c1c(Cl)nc(C=O)c(-c2ccnc(F)c2)c1N1CCC(C)(C)CC1]

### Ethyl (S)-2-(tert-butoxy)-2-(6-chloro-4-(4,4-dimethylpiperidin-1-yl)-5′-fluoro-2-formyl-[3,3′-bipyridin]-5-yl)acetate

To a dry reaction vial under argon was added ethyl-(S)-2-(5-bromo-2-chloro-4-(4,4-dimethylpiperidin-1-yl)-6-formylpyridin-3-yl)-2-(tert-butoxy)acetate (104 mg, 0.212 mmol), (5-fluoropyridin-3-yl)boronic acid (66 mg, 0.468 mmol) and THF (9 mL). The reaction was flushed with argon, treated with 0.5 M potassium phosphate tribasic (1.80 mL, 0.900 mmol), followed by 2^{nd} generation X-phos precatalyst (18 mg, 0.023 mmol), capped and stirred at room temp for 48 h. The reaction was diluted with ethyl acetate, extracted and the crude residue was purified via silica gel chromatography (24 g SiO₂ column, dichloromethane:EtOAc 100:0->30:70) to afford ethyl (S)-2-(tert-butoxy)-2-(6-chloro-4-(4,4-dimethylpiperidin-1-yl)-5′-fluoro-2-formyl-[3,3′-bipyridin]-5-yl)acetate, 14 mg (13%). LCMS (M+1)=506.3.

Also present within the above intermediate was

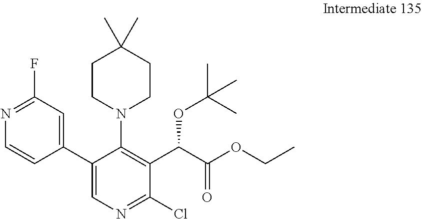 [SMILES: CCOC(=O)[C@@H](OC(C)(C)C)c1c(Cl)ncc(-c2ccnc(F)c2)c1N1CCC(C)(C)CC1]

### Ethyl (S)-2-(tert-butoxy)-2-(6-chloro-4-(4, 4-dimethylpiperidin-1-yl)-5′-fluoro-[3, 3′-bipyridin]-5-yl)acetate

LCMS (M+1)=478.2 and 480.2.

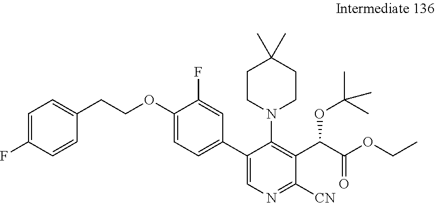 [SMILES: CCOC(=O)[C@@H](OC(C)(C)C)c1c(C#N)ncc(-c2ccc(OCCc3ccc(F)cc3)c(F)c2)c1N1CCC(C)(C)CC1]

### Ethyl (S)-2-(tert-butoxy)-2-(2-cyano-4-(4, 4-dimethylpiperidin-1-yl)-5-(3-fluoro-4-(4-fluorophenethoxy)phenyl)pyridin-3-yl)acetate

To a dry microwave vial under nitrogen was added ethyl-(S)-2-(tert-butoxy)-2-(2-chloro-4-(4,4-dimethylpiperidin-1-yl)-5-(3-fluoro-4-(4-fluorophenethoxy)phenyl)pyridin-3-yl)acetate (113.7 mg, 0.185 mmol), zinc (3 mg, 0.046 mmol), zinc cyanide (45 mg, 0.383 mmol) and DMF (1.0 mL). The reaction was flushed with argon, treated with [1,1′-bis(diphenylphosphino)ferrocene]dichloropalladium(II) (13 mg, 0.018 mmol), capped and heated in a microwave reactor at 145 C for 19 h. The reaction was diluted with ethyl acetate (100 mL), extracted with water (3×15 mL), brine (1×50 mL), dried over Na₂SO₄ and concentrated. The crude material was purified via silica gel chromatography (40 g SiO₂ column, hexane:EtOAc 100:0->60:40) to afford ethyl (S)-2-(tert-butoxy)-2-(2-cyano-4-(4,4-dimethylpiperidin-1-yl)-5-(3-fluoro-4-(4-fluorophenethoxy)phenyl)pyridin-3-yl)acetate, 63.1 mg (56%). LCMS (M+1)=606.4.

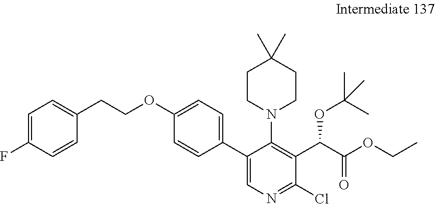 [SMILES: CCOC(=O)[C@@H](OC(C)(C)C)c1c(Cl)ncc(-c2ccc(OCCc3ccc(F)cc3)cc2)c1N1CCC(C)(C)CC1]

### (S)-Ethyl 2-(tert-butoxy)-2-(2-chloro-4-(4, 4-dimethylpiperidin-1-yl)-5-(4-(4-fluorophenethoxy)phenyl)pyridin-3-yl)acetate

A mixture of (S)-ethyl 2-(5-bromo-2-chloro-4-(4,4-dimethylpiperidin-1-yl)pyridin-3-yl)-2-(tert-butoxy)acetate (230 mg, 0.498 mmol) and (4-(4-fluorophenethoxy)phenyl)boronic acid in THF (25 mL) is flushed well with argon, then added degassed 0.5 M potassium phosphate tribasic (3 mL, 1.500 mmol) then 2nd generation Xphos precatalyst (30 mg, 0.038 mmol), sealed vial and stirred at RT for 16 hours overnight. LC/MS showed formation of the ester. The organic layer was decanted from the water layer, removed the THF under nitrogen, take up in EtOAc, dry over MgSO₄, filter, and concentrate under vacuum. Purified residue by flash column chromatography to give 260 mg (93%) of (S)-ethyl 2-(tert-butoxy)-2-(2-chloro-4-(4,4-dimethylpiperidin-1-yl)-5-(4-(4-fluorophenethoxy)phenyl)pyridin-3-yl)acetate as a colorless solid. LCMS (M+1)=596.7.

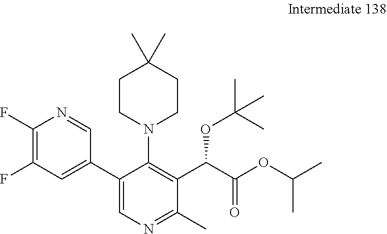 [SMILES: Cc1ncc(-c2cnc(F)c(F)c2)c(N2CCC(C)(C)CC2)c1[C@H](OC(C)(C)C)C(=O)OC(C)C]

### Isopropyl (S)-2-(tert-butoxy)-2-(4-(4, 4-dimethylpiperidin-1-yl)-5′,6′-difluoro-6-methyl-[3,3′-bipyridin]-5-yl)acetate

To a 100 mL Schlenk flask equipped with a stir bar was added dioxane (40 ml) and water (10.00 ml). The flask was sealed with a septum, then the solution was degassed via N₂ sparging for 10 min. To the solution was added (5,6-difluoropyridin-3-yl)boronic acid (2.00 g, 12.6 mmol), isopropyl (S)-2-(5-bromo-4-(4,4-dimethylpiperidin-1-yl)-2-methylpyridin-3-yl)-2-(tert-butoxy)acetate (3.82 g, 8.39 mmol), tribasic potassium phosphate (8.02 g, 37.8 mmol), and SPhos-Pd-G3 (0.327 g, 0.420 mmol). The flask was placed in a 60° C. heating bath with stirring for 4 h. The reaction mixture was cooled to r.t., then was transferred to a 500 mL separatory funnel and was diluted with water (200 mL), then was extracted with Et₂O (200 mL). The organic phase was dried over MgSO₄; filtered; then concentrated in vacuo. The residue was dissolved in a min of acetone, then was concentrated onto Celite in vacuo. The resulting powder was subjected to SiO₂ chromatography (80 g SiO₂ column, hexanes:EtOAc 100:0460:40) to afford isopropyl (S)-2-(tert-butoxy)-2-(4-(4,4-dimethylpiperidin-1-yl)-5′,6′-difluoro-6-methyl-[3,3′-bipyridin]-5-yl)acetate as a solid yellow foam (3.5305 g, 86%). ¹H NMR (500 MHz, CHLOROFORM-d) δ 8.14 (s, 1H), 7.95 (t, J=1.8 Hz, 1H), 7.54 (t, J=8.4 Hz, 1H), 5.91 (br s, 1H), 5.12 (spt, J=6.3 Hz, 1H), 3.43 (br s, 1H), 2.96 (br s, 1H), 2.64 (s, 3H), 2.43-2.22 (m, 2H), 1.26 (d, J=6.3 Hz, 3H), 1.24 (d, J=6.1 Hz, 3H), 1.18 (s, 9H), 1.50-1.12 (m, 4H), 0.94 (br s, 3H), 0.85 (br s, 3H).

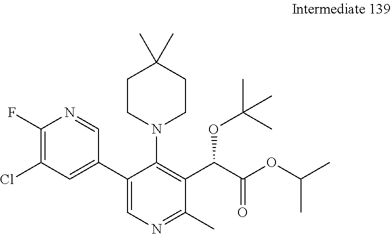 [SMILES: Cc1ncc(-c2cnc(F)c(Cl)c2)c(N2CCC(C)(C)CC2)c1[C@H](OC(C)(C)C)C(=O)OC(C)C]

### Isopropyl (S)-2-(tert-butoxy)-2-(5′-chloro-4-(4, 4-dimethylpiperidin-1-yl)-6′-fluoro-6-methyl-[3, 3′-bipyridin]-5-yl)acetate

A mixture of Isopropyl (S)-2-(5-bromo-4-(4,4-dimethylpiperidin-1-yl)-2-methylpyridin-3-yl)-2-(tert-butoxy)acetate (0.100 g, 0.220 mmol, 1 equiv), 3-chloro-2-fluoro-5-(4,4,5,5-tetramethyl-1,3,2-dioxaborolan-2-yl)pyridine (0.057 g, 0.220 mmol, 1 equiv), Cs₂CO₃ (0.300 g, 0.922 mmol, 4.2 equiv), and Pd(dppf)Cl₂ CH₂Cl₂ adduct (0.018 g, 0.022 mmol, 0.1 equiv) in dioxane was heated at 110° C. for 2 h. After cooling to ambient temperature, the reaction mixture was filtered through celite/Na₂SO₄ eluting with ethyl acetate. The filtrate was concentrated in vacuo and purified by silica gel flash chromatography (0-50% ethyl acetate in hexanes) to give the product (0.069 g, 62%) as a white foamy solid. LCMS (M+1): 506.35.

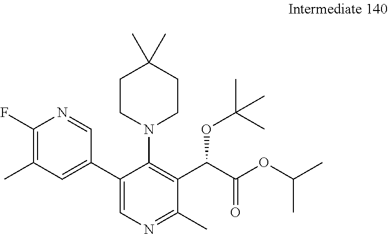 [SMILES: Cc1cc(-c2cnc(C)c([C@H](OC(C)(C)C)C(=O)OC(C)C)c2N2CCC(C)(C)CC2)cnc1F]

### Isopropyl (S)-2-(tert-butoxy)-2-(4-(4, 4-dimethylpiperidin-1-yl)-6′-fluoro-5′,6-dimethyl-[3,3′-bipyridin]-5-yl)acetate

A solution of isopropyl (S)-2-(5-bromo-4-(4,4-dimethylpiperidin-1-yl)-2-methylpyridin-3-yl)-2-(tert-butoxy)acetate (0.030 g, 0.066 mmol, 1 equiv), (6-fluoro-5-methylpyridin-3-yl)boronic acid (10.21 mg, 0.066 mmol, 1 equiv), Cs₂CO₃ (0.090 g, 0.277 mmol, 4.2 equiv), and Pd(dppf)Cl₂ CH₂Cl₂ adduct (5.38 mg, 6.59 μmol, 0.1 equiv) in dioxane was heated at 110° C. for 2 h. After cooling to ambient temperature, the reaction was filtered through a plug of celite and concentrated in vacuo. The crude product was carried on without further purification. LCMS (M+1): 486.4.

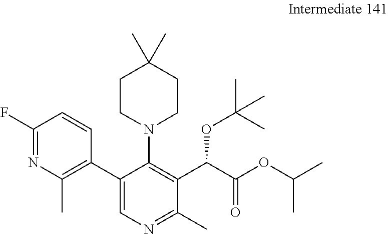 [SMILES: Cc1nc(F)ccc1-c1cnc(C)c([C@H](OC(C)(C)C)C(=O)OC(C)C)c1N1CCC(C)(C)CC1]

### Isopropyl (S)-2-(tert-butoxy)-2-(4-(4, 4-dimethylpiperidin-1-yl)-6′-fluoro-2′,6-dimethyl-[3, 3′-bipyridin]-5-yl)acetate

A mixture of isopropyl (S)-2-(5-bromo-4-(4,4-dimethylpiperidin-1-yl)-2-methylpyridin-3-yl)-2-(tert-butoxy)acetate (0.15 g, 0.329 mmol, 1 equiv), (6-fluoro-2-methylpyridin-3-yl)boronic acid (0.102 g, 0.659 mmol, 2 equiv), Cs₂CO₃ (0.451 g, 1.383 mmol, 4.2 equiv), and Pd(dppf)Cl₂ CH₂Cl₂ adduct (0.027 g, 0.033 mmol, 0.1 equiv) in dioxane (5 mL) was heated at 110° C. for 2 h. After cooling to ambient temperature, the reaction was concentrated in vacuo and purified silica gel flash chromatography (0-100% ethyl acetate/hexanes) to give the product (42 mg, 26%) as a pale yellow viscous oil. LCMS (M+1): 486.40.

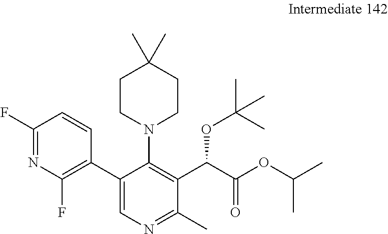 [SMILES: Cc1ncc(-c2ccc(F)nc2F)c(N2CCC(C)(C)CC2)c1[C@H](OC(C)(C)C)C(=O)OC(C)C]

### Isopropyl (S)-2-(tert-butoxy)-2-(4-(4, 4-dimethylpiperidin-1-yl)-2′,6′-difluoro-6-methyl-[3,3′-bipyridin]-5-yl)acetate

A mixture of isopropyl (S)-2-(5-bromo-4-(4,4-dimethylpiperidin-1-yl)-2-methylpyridin-3-yl)-2-(tert-butoxy)acetate (0.15 g, 0.329 mmol, 1 equiv), (2,6-difluoropyridin-3-yl)boronic acid (0.105 g, 0.659 mmol, 2 equiv), Cs₂CO₃ (0.451 g, 1.383 mmol, 4.2 equiv), and Pd(dppf)Cl₂ CH₂Cl₂ adduct (0.027 g, 0.033 mmol, 0.1 equiv) in dioxane (5 mL) was heated at 110° C. for 2 h. After cooling to ambient temperature, the reaction was concentrated in vacuo and purified by silica gel flash chromatography (0-100% ethyl acetate/hexanes to give the product (22 mg, 14%) as a pale yellow viscous oil. LCMS (M+1): 490.4.

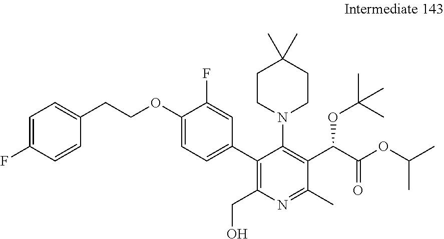 [SMILES: Cc1nc(CO)c(-c2ccc(OCCc3ccc(F)cc3)c(F)c2)c(N2CCC(C)(C)CC2)c1[C@H](OC(C)(C)C)C(=O)OC(C)C]

### Isopropyl (S)-2-(tert-butoxy)-2-(4-(4, 4-dimethylpiperidin-1-yl)-5-(3-fluoro-4-(4-fluorophenethoxy)phenyl)-6-(hydroxymethyl)-2-methylpyridin-3-yl)acetate

To a dry 150 mL pressure bottle under nitrogen was added isopropyl (S)-2-(5-bromo-4-(4,4-dimethylpiperidin-1-yl)-6-(hydroxymethyl)-2-methylpyridin-3-yl)-2-(tert-butoxy)acetate (630 mg, 1.298 mmol), 2-(3-fluoro-4-(4-fluorophenethoxy)phenyl)-4,4,5,5-tetramethyl-1,3,2-dioxaborolane (700 mg, 1.943 mmol) and THF (60 mL). The reaction was flushed well with argon, treated with potassium phosphate tribasic, 0.5M in water (9.75 mL, 4.88 mmol), followed by 2^{nd} generation X-phos precatalyst (71.1 mg, 0.090 mmol), capped and stirred at room temp for 18 h. The crude reaction was diluted with ethyl acetate (300 mL), extracted with water (1×10 mL), brine (1×10 mL), dried over Na₂SO₄ and concentrated. The crude material was purified via silica gel chromatography (80 g SiO₂ column, hexane:EtOAc 100:0->50:50) to afford isopropyl (S)-2-(tert-butoxy)-2-(4-(4,4-dimethylpiperidin-1-yl)-5-(3-fluoro-4-(4-fluorophenethoxy)phenyl)-6-(hydroxymethyl)-2-methylpyridin-3-yl)acetate, 560 mg (54%). LCMS=639.4 (M+H).

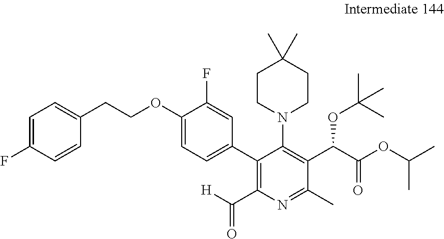 [SMILES: Cc1nc(C=O)c(-c2ccc(OCCc3ccc(F)cc3)c(F)c2)c(N2CCC(C)(C)CC2)c1[C@H](OC(C)(C)C)C(=O)OC(C)C]

### Isopropyl (S)-2-(tert-butoxy)-2-(4-(4, 4-dimethylpiperidin-1-yl)-5-(3-fluoro-4-(4-fluorophenethoxy)phenyl)-6-formyl-2-methylpyridin-3-yl)acetate

To a solution of isopropyl (S)-2-(tert-butoxy)-2-(4-(4,4-dimethylpiperidin-1-yl)-5-(3-fluoro-4-(4-fluorophenethoxy)phenyl)-6-(hydroxymethyl)-2-methylpyridin-3-yl)acetate (477.3 mg, 0.747 mmol) in a mixture of CH₂Cl₂ (20 mL) and acetonitrile (2.0 mL) was added Dess-Martin periodinane (514 mg, 1.212 mmol). The reaction was flushed briefly with N₂, capped and stirred at room temp for 4.5 h. The crude reaction was diluted with Et₂O (400 mL), extracted with 1.0M NaOH (2×40 mL), brine (1×20 mL), dried over Na₂SO₄, filtered and concentrated. The crude material was purified via silica gel chromatography (40 g SiO₂ column, CH₂Cl₂:EtOAc 100:0->20:80) to afford isopropyl (S)-2-(tert-butoxy)-2-(4-(4,4-dimethylpiperidin-1-yl)-5-(3-fluoro-4-(4-fluorophenethoxy)phenyl)-6-formyl-2-methylpyridin-3-yl)acetate, 458 mg (96%). LCMS=668.7 (M+32).

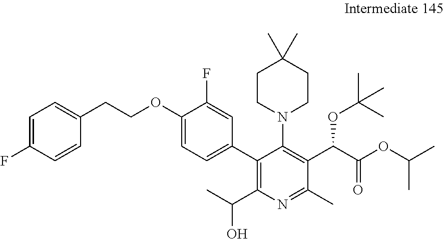 [SMILES: Cc1nc(C(C)O)c(-c2ccc(OCCc3ccc(F)cc3)c(F)c2)c(N2CCC(C)(C)CC2)c1[C@H](OC(C)(C)C)C(=O)OC(C)C]

### Isopropyl (2S)-2-(tert-butoxy)-2-(4-(4, 4-dimethylpiperidin-1-yl)-5-(3-fluoro-4-(4-fluorophenethoxy)phenyl)-6-(1-hydroxyethyl)-2-methylpyridin-3-yl)acetate (Atrop Isomer 1)

To a dry reaction vial under N₂ was added isopropyl (S)-2-(tert-butoxy)-2-(4-(4,4-dimethylpiperidin-1-yl)-5-(3-fluoro-4-(4-fluorophenethoxy)phenyl)-6-formyl-2-methylpyridin-3-yl)acetate (40 mg, 0.063 mmol) and THF (1.5 mL). The reaction was flushed very well with argon, cooled to 0° C. and treated with methylmagnesium chloride, 3.0 M in THF (25 μL, 0.075 mmol) over 30 seconds. The reaction was stirred at 0° C. for 2 min, then the bath was removed and the reaction was allowed to slowly warm to room temp over 10 min. The reaction was recooled to 0° C. and treated with additional methylmagnesium chloride, 3.0 M in THF (25 μL, 0.075 mmol). The cooling bath was removed and the reaction was allowed to warm to room temp over 15 min. The crude material was purified via silica gel chromatography (40 g SiO₂ column, CH₂Cl₂:EtOAc 100:0->50:50) to afford isopropyl (2S)-2-(tert-butoxy)-2-(4-(4,4-dimethylpiperidin-1-yl)-5-(3-fluoro-4-(4-fluorophenethoxy)phenyl)-6-(1-hydroxyethyl)-2-methylpyridin-3-yl)acetate (diastereomer 1), 15.3 mg (37%). LCMS=652.7 (M+H).

Also isolated from this reaction was

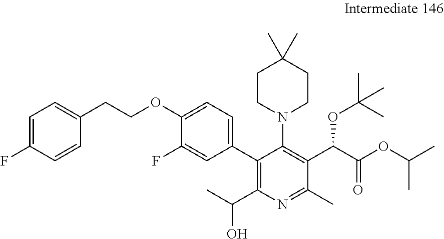 [SMILES: Cc1nc(C(C)O)c(-c2ccc(OCCc3ccc(F)cc3)c(F)c2)c(N2CCC(C)(C)CC2)c1[C@H](OC(C)(C)C)C(=O)OC(C)C]

Isopropyl (2S)-2-(tert-butoxy)-2-(4-(4,4-dimethylpiperidin-1-yl)-5-(3-fluoro-4-(4-fluorophenethoxy)phenyl)-6-(1-hydroxyethyl)-2-methylpyridin-3-yl)acetate (diastereomer 2), 9.7 mg (24%). LCMS=652.7 (M+H).

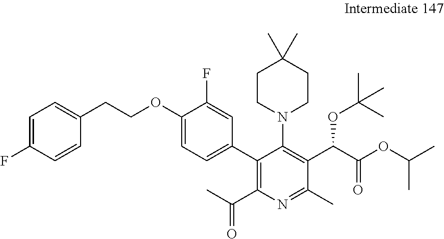 [SMILES: CC(=O)c1nc(C)c([C@H](OC(C)(C)C)C(=O)OC(C)C)c(N2CCC(C)(C)CC2)c1-c1ccc(OCCc2ccc(F)cc2)c(F)c1]

### Isopropyl (S)-2-(6-acetyl-4-(4, 4-dimethylpiperidin-1-yl)-5-(3-fluoro-4-(4-fluorophenethoxy)phenyl)-2-methylpyridin-3-yl)-2-(tert-butoxy)acetate

To a dry reaction vial under N₂ was added isopropyl (2S)-2-(tert-butoxy)-2-(4-(4,4-dimethylpiperidin-1-yl)-5-(3-fluoro-4-(4-fluorophenethoxy)phenyl)-6-(1-hydroxyethyl)-2-methylpyridin-3-yl)acetate (111.5 mg, 0.171 mmol), CH₂Cl₂ (4 mL), acetonitrile (0.4 mL) and several pieces of 4 A° molecular sieves. The reaction was then treated with Dess-Martin periodinane (129.3 mg, 0.305 mmol), capped and stirred at room temp for 18 h. The reaction was diluted with EtOAc (150 mL), extracted with aq 1M NaOH (1×5 mL), water (1×5 mL), brine (1×5 mL), dried over Na₂SO₄ and concentrated. The crude material was purified via silica gel chromatography (24 g SiO₂ column, CH₂Cl₂:EtOAc 100:0->85:15) to afford isopropyl (S)-2-(6-acetyl-4-(4,4-dimethylpiperidin-1-yl)-5-(3-fluoro-4-(4-fluorophenethoxy)phenyl)-2-methylpyridin-3-yl)-2-(tert-butoxy)acetate, 100 mg (90%). LC/MS=651.4 (M+H).

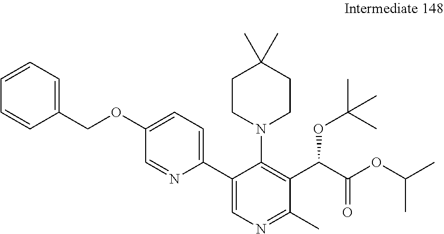 [SMILES: Cc1ncc(-c2ccc(OCc3ccccc3)cn2)c(N2CCC(C)(C)CC2)c1[C@H](OC(C)(C)C)C(=O)OC(C)C]

### Isopropyl (S)-2-(5-(benzyloxy)-4′-(4, 4-dimethylpiperidin-1-yl)-6′-methyl-[2, 3′-bipyridin]-5′-yl)-2-(tert-butoxy)acetate

To a 100 mL Schlenk flask equipped with a stir bar was added 2-(5-(benzyloxy)pyridin-2-yl)-6-methyl-1,3,6,2-dioxazaborocane-4,8-dione (3.50 g, 10.29 mmol), isopropyl (S)-2-(5-bromo-4-(4,4-dimethylpiperidin-1-yl)-2-methylpyridin-3-yl)-2-(tert-butoxy)acetate (3.12 g, 6.86 mmol), palladium tetrakis (1.585 g, 1.372 mmol), diacetoxycopper (0.623 g, 3.43 mmol) and anhydrous tribasic potassium phosphate, finely ground (7.28 g, 34.3 mmol). The flask was sealed with a rubber septum, then placed under N₂ atm. To the flask was added a degassed (N₂ sparging for 5 min) solution of diethanolamine (0.721 g, 6.86 mmol) in DMF (60 mL). The flask was placed in a 100° C. oil bath with stirring for 18 h. The reaction mixture was transferred to a 1 L separatory funnel. The mixture was diluted with water:brine and extracted with EtOAc. However, an emulsion made this process extremely problematic. The volumes of each solvent were increased incrementally to finally achieve water (175 mL):brine (175 mL):EtOAc (250 mL). However, the emulsion persisted. The entire mixture was filtered through Celite. The filtrate was transferred back to the separatory funnel and the mixture was shaken, upon which the emulsion re-formed and persisted. The mixture was filtered through Celite and the mixture was immediately partitioned in the separatory funnel without further mixing. The aq. phase was mixed with EtOAc (250 mL). The emulsion was filtered through the same Celite pad as before, and the mixture was partitioned in the separatory funnel without further mixing. The combined organics were washed with water:brine (175 mL: 175 mL) (no problematic emulsion), then brine (150 mL) (no problematic emulsion). The combined organics were dried over MgSO₄; were filtered; then were concentrated in vacuo. The resulting amber solution/solid residue was dissolved in a min. of acetone, then was concentrated onto Celite in vacuo. The resulting powder was subjected to SiO₂ chromatography (220 g SiO₂ column, hexanes:EtOAc 100:0→40:60) to afford isopropyl (S)-2-(5-(benzyloxy)-4′-(4,4-dimethylpiperidin-1-yl)-6′-methyl-[2,3′-bipyridin]-5′-yl)-2-(tert-butoxy)acetate as a colorless solid (1.88 g, 49%). ¹H NMR (500 MHz, CHLOROFORM-d) δ 8.51 (d, J=2.4 Hz, 1H), 8.28 (s, 1H), 7.51-7.36 (m, 7H), 7.32 (d, J=8.5 Hz, 1H), 6.06 (br s, 1H), 5.24-5.18 (m, 2H), 5.16-5.09 (m, 1H), 3.31 (br d, J=11.3 Hz, 2H), 2.64 (s, 3H), 2.34-2.07 (m, 1H), 1.48-1.27 (m, 4H), 1.25 (d, J=6.3 Hz, 3H), 1.22 (d, J=6.1 Hz, 3H), 1.19 (s, 9H), 0.98-0.89 (m, 3H), 0.82 (br s, 3H).

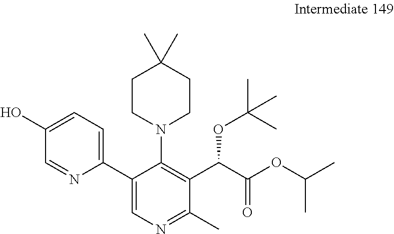 [SMILES: Cc1ncc(-c2ccc(O)cn2)c(N2CCC(C)(C)CC2)c1[C@H](OC(C)(C)C)C(=O)OC(C)C]

### Isopropyl (S)-2-(tert-butoxy)-2-(4′-(4, 4-dimethylpiperidin-1-yl)-5-hydroxy-6′-methyl-[2, 3′-bipyridin]-5′-yl)acetate

To a 250 mL round bottom flask equipped with a stir bar and charged with isopropyl (S)-2-(5-(benzyloxy)-4′-(4,4-dimethylpiperidin-1-yl)-6′-methyl-[2,3′-bipyridin]-5′-yl)-2-(tert-butoxy)acetate (1.8796 g, 3.36 mmol) was added palladium on carbon (Degussa type E101 NE/W, 10% dry basis Pd, 50% wt water, 0.179 g, 0.168 mmol) and MeOH (20 mL). The flask was sealed with a rubber septum, then was sparged with N₂ for 5 min. The mixture was then sparged with H₂ for 3 minutes, then was placed under a static balloon-pressure H₂ atm. with stirring for 1 h. The mixture was sparged with N₂ for 10 minutes, then the reaction mixture was directly concentrated onto Celite in vacuo. The resulting powder was subjected to SiO₂ chromatography (DCM:EtOH 100:0→97.5:2.5→90:10) to a colorless solid. The material was co-evaporated twice with PhMe to afford isopropyl (S)-2-(tert-butoxy)-2-(4′-(4,4-dimethylpiperidin-1-yl)-5-hydroxy-6′-methyl-[2,3′-bipyridin]-5′-yl)acetate as a colorless solid foam (1.41 g, 80% yield). ¹H NMR (500 MHz, CHLOROFORM-d) δ 8.40 (d, J=2.7 Hz, 1H), 8.22 (s, 1H), 7.31-7.26 (m, 1H), 7.22-7.16 (m, 1H), 6.03 (br s, 1H), 5.14 (quin, J=6.3 Hz, 1H), 3.51-3.09 (m, 2H), 2.71 (s, 3H), 2.44-2.18 (m, 2H), 1.77-1.42 (m, 2H), 1.41-1.31 (m, 4H), 1.26 (d, J=6.1 Hz, 3H), 1.23 (d, J=6.3 Hz, 3H), 1.20 (s, 9H), 0.90 (br s, 6H).

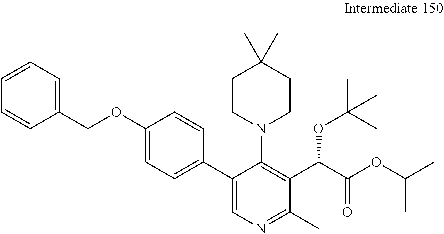 [SMILES: Cc1ncc(-c2ccc(OCc3ccccc3)cc2)c(N2CCC(C)(C)CC2)c1[C@H](OC(C)(C)C)C(=O)OC(C)C]

### Isopropyl (S)-2-(5-(4-(benzyloxy)phenyl)-4-(4, 4-dimethylpiperidin-1-yl)-2-methylpyridin-3-yl)-2-(tert-butoxy)acetate

To a 40 mL vial equipped with a stir was added tribasic potassium phosphate (anhydrous, 4.19 g, 19.76 mmol), (4-(benzyloxy)phenyl)boronic acid (0.751 g, 3.29 mmol), isopropyl (S)-2-(5-bromo-4-(4,4-dimethylpiperidin-1-yl)-2-methylpyridin-3-yl)-2-(tert-butoxy)acetate (1.0 g, 2.196 mmol), and SPhos-Pd-G3 (0.086 g, 0.110 mmol). The vial was sealed with a screw cap rubber septum and then placed under N₂ atm. To the vial was added a degassed (N₂ bubbling for 5 minutes) solution of dioxane:water (15 mL:5 mL) water (5 mL). The vial was placed in a 65° C. heating block with stirring for 2 h. The reaction mixture was transferred to 500 mL separatory funnel and was diluted with Et₂O (250 mL). The mixture was washed with water (250 mL), then dried over MgSO₄; filtered; then concentrated in vacuo. The resulting residue was dissolved in a min. of acetone, then was concentrated onto Celite in vacuo. The resulting powder was subjected to SiO₂ purification (hexanes:EtOAc 90:10-40:60) to afford a colorless solid foam, isopropyl (S)-2-(5-(4-(benzyloxy)phenyl)-4-(4,4-dimethylpiperidin-1-yl)-2-methylpyridin-3-yl)-2-(tert-butoxy)acetate (1.0882 g, 89% yield). ¹H NMR (500 MHz, CHLOROFORM-d) δ 8.18 (s, 1H), 7.49 (d, J=7.4 Hz, 2H), 7.43 (t, J=7.6 Hz, 2H), 7.40-7.35 (m, 1H), 7.23 (d, J=8.4 Hz, 2H), 7.06 (d, J=8.5 Hz, 2H), 6.02 (br s, 1H), 5.16 (s, 2H), 5.14-5.08 (m, 1H), 3.37 (br s, 1H), 3.02 (br s, 1H), 2.63 (s, 3H), 2.31 (br s, 1H), 2.22 (br s, 1H), 1.25 (dd, J=11.6, 6.2 Hz, 6H), 1.19 (s, 9H), 0.93 (br s, 3H), 0.77 (br s, 3H); 4 piperidine protons appear as very broad resonances between 0.7 and 1.7 ppm.

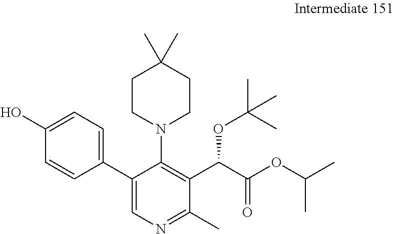 [SMILES: Cc1ncc(-c2ccc(O)cc2)c(N2CCC(C)(C)CC2)c1[C@H](OC(C)(C)C)C(=O)OC(C)C]

### Isopropyl (S)-2-(tert-butoxy)-2-(4-(4, 4-dimethylpiperidin-1-yl)-5-(4-hydroxyphenyl)-2-methylpyridin-3-yl)acetate

To a 100 mL round bottom flask equipped with a stir bar and charged with isopropyl (S)-2-(5-(4-(benzyloxy)phenyl)-4-(4,4-dimethylpiperidin-1-yl)-2-methylpyridin-3-yl)-2-(tert-butoxy)acetate (1.0882 g, 1.948 mmol) was added 10% Pd—C (0.104 g, 0.097 mmol) and MeOH (20 mL). The flask was sealed with a rubber septum, then the mixture was sparged via N₂ bubbling through the solvent for 5 min. The mixture was then sparged with H₂ for 3 minutes. The mixture was placed under a static balloon-pressure H₂ atm. with stirring for 30 min. The atmosphere was exchanged to N₂ (5 min bubbling). The reaction mixture was filtered through a pad of Celite and the filtrate was concentrated onto Celite in vacuo. The resulting powder was subjected to SiO₂ purification (hexanes:EtOAc 100:0-0:100) to afford a colorless solid, isopropyl (S)-2-(tert-butoxy)-2-(4-(4,4-dimethylpiperidin-1-yl)-5-(4-hydroxyphenyl)-2-methylpyridin-3-yl)acetate (784 mg, 85% yield). ¹H NMR (500 MHz, CHLOROFORM-d) δ 8.15 (s, 1H), 7.15-7.11 (m, 2H), 6.96-6.91 (m, 2H), 6.00 (br s, 1H), 5.11 (spt, J=6.3 Hz, 1H), 3.36 (br s, 1H), 3.01 (br s, 1H), 2.64 (s, 3H), 2.39 (br s, 1H), 2.23 (br s, 1H), 1.62 (br s, 4H), 1.24 (dd, J=11.1, 6.2 Hz, 6H), 1.18 (s, 9H), 0.96-0.74 (m, 6H).

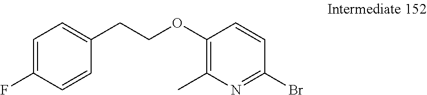 [SMILES: Cc1nc(Br)ccc1OCCc1ccc(F)cc1]

### 6-Bromo-3-(4-fluorophenethoxy)-2-methylpyridine

To a 50 mL round bottom flask equipped with a stir bar was added 6-bromo-2-methylpyridin-3-ol (250 mg, 1.330 mmol), 2-(4-fluorophenyl)ethan-1-ol (186 mg, 1.330 mmol), triphenylphosphine (418 mg, 1.596 mmol) and THF (10 mL). To the stirred solution was added DIAD (0.310 mL, 1.596 mmol). The solution warmed to a mild reflux, then cooled within 5 minutes. The solution was stirred at r.t. for 2 hrs. The reaction solution was concentrated in vacuo and the resulting oil was diluted with a min of acetone, then concentrated onto Celite in vacuo. The resulting powder was subjected to silica gel chromatography (40 g column, 5-40% EtOAc:Hex) to afford the product 6-bromo-3-(4-fluorophenethoxy)-2-methylpyridine (386 mg, 1.244 mmol, 94% yield) as a white solid. ¹H NMR (500 MHz, chloroform-d) δ 7.29-7.21 (m, 3H), 7.03 (t, J=8.7 Hz, 2H), 6.95 (d, J=8.5 Hz, 1H), 4.14 (t, J=6.6 Hz, 2H), 3.11 (t, J=6.5 Hz, 2H), 2.42 (s, 3H). ESI-MS(+) m/z=309.9 (M+1).

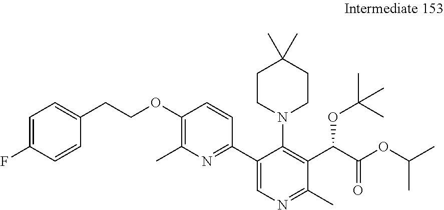 [SMILES: Cc1nc(-c2cnc(C)c([C@H](OC(C)(C)C)C(=O)OC(C)C)c2N2CCC(C)(C)CC2)ccc1OCCc1ccc(F)cc1]

### Isopropyl (S)-2-(tert-butoxy)-2-(4′-(4, 4-dimethylpiperidin-1-yl)-5-(4-fluorophenethoxy)-6, 6′-dimethyl-[2, 3′-bipyridin]-5′-yl)acetate

To a 14 mL test tube equipped with a stir bar was added (S)-(5-(1-(tert-butoxy)-2-isopropoxy-2-oxoethyl)-4-(4,4-dimethylpiperidin-1-yl)-6-methylpyridin-3-yl)boronic acid (100 mg, 0.238 mmol), SPhos-Pd-G3 (9.27 mg, 0.012 mmol), tribasic potassium phosphate (454 mg, 2.141 mmol) and 6-bromo-3-(4-fluorophenethoxy)-2-methylpyridine (73.8 mg, 0.238 mmol). The flask was sealed with a rubber septum, then was placed under N₂ atm (vac/fill×3). To the flask was added degassed (N₂ bubbling for 5 min) Dioxane (892 μl) and Water (297 μl). The test tube was placed in a 60° C. heating block with stirring (t=0). LCMS analysis at t=3 h found a large product mass peak and the disappearance of the starting boronic acid. The reaction was cooled to RT and diluted with EtOAc and water. The organic layer was washed with brine, collected, dried over MgSO₄, filtered and volatiles evaporated to afford the crude product. The crude product was purified via silica gel chromatography (24 g column, 20-100% EtOAc:Hex) to afford the product isopropyl (S)-2-(tert-butoxy)-2-(4′-(4,4-dimethylpiperidin-1-yl)-5-(4-fluorophenethoxy)-6,6′-dimethyl-[2,3′-bipyridin]-5′-yl)acetate (20 mg, 0.033 mmol, 13.88% yield) as a brown oil. ESI-MS(+) m/z=606.8 (M+1).

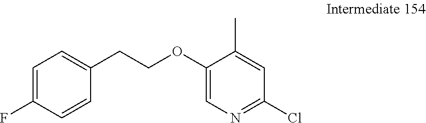 [SMILES: Cc1cc(Cl)ncc1OCCc1ccc(F)cc1]

### 2-Chloro-5-(4-fluorophenethoxy)-4-methylpyridine

To a 40 mL vial equipped with a stir bar was added 6-chloro-4-methylpyridin-3-ol (1.0 g, 6.97 mmol), 2-(4-fluorophenyl)ethan-1-ol (0.976 g, 6.97 mmol), triphenylphosphine (2.192 g, 8.36 mmol) and THF (25 mL). To the stirred solution was added DIAD (1.625 mL, 8.36 mmol). The solution warmed to a mild reflux, then cooled within 5 minutes. The solution was stirred at r.t. (t=0). LCMS analysis at t=18 h found a major peak corresponding to the desired product. The reaction solution (blue color) was transferred to a 100 mL r.b. flask and then concentrated in vacuo. The resulting residue was dissolved in a min of acetone, then was concentrated onto Celite in vacuo. The resulting powder was subjected to SiO₂ purification (80 g column, 0-20% EtOAc:Hex) to afford the product 2-chloro-5-(4-fluorophenethoxy)-4-methylpyridine (1.404 g, 5.28 mmol, 76% yield) as a colorless oil. ¹H NMR (500 MHz, chloroform-d) δ 7.85 (s, 1H), 7.25-7.21 (m, 2H), 7.07 (s, 1H), 7.04-6.98 (m, 2H), 4.21 (t, J=6.5 Hz, 2H), 3.09 (t, J=6.5 Hz, 2H), 2.16 (s, 3H).

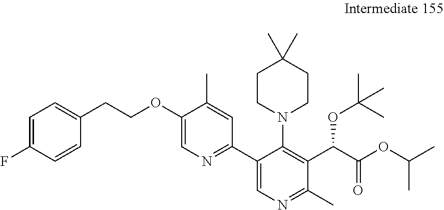 [SMILES: Cc1cc(-c2cnc(C)c([C@H](OC(C)(C)C)C(=O)OC(C)C)c2N2CCC(C)(C)CC2)ncc1OCCc1ccc(F)cc1]

### Isopropyl (S)-2-(tert-butoxy)-2-(4′-(4, 4-dimethylpiperidin-1-yl)-5-(4-fluorophenethoxy)-4, 6′-dimethyl-[2, 3′-bipyridin]-5′-yl)acetate

To a 14 mL test tube equipped with a stir bar and (S)-(5-(1-(tert-butoxy)-2-isopropoxy-2-oxoethyl)-4-(4,4-dimethylpiperidin-1-yl)-6-methylpyridin-3-yl)boronic acid (100 mg, 0.238 mmol) was added SPhos-Pd-G3 (9.27 mg, 0.012 mmol), tribasic potassium phosphate (454 mg, 2.141 mmol) and 2-chloro-5-(4-fluorophenethoxy)-4-methylpyridine (63.2 mg, 0.238 mmol). The flask was sealed with a rubber septum, then was placed under N₂ atm (vac/fill×3). To the flask was added degassed (N₂ bubbling for 5 min) dioxane (892 μl) and water (297 μl). The test tube was placed in a 60° C. heating block with stirring (t=0). LCMS analysis at t=3 h found a large product mass peak and the disappearance of the starting boronic acid. The reaction was cooled to RT and diluted with EtOAc and water. The organic layer was washed with brine, collected, dried over MgSO₄, filtered and volatiles evaporated to afford the crude product. The crude product was purified via silica gel chromatography (24 g column, 20-100% EtOAc:Hex) to afford the product isopropyl (S)-2-(tert-butoxy)-2-(4′-(4,4-dimethylpiperidin-1-yl)-5-(4-fluorophenethoxy)-4,6′-dimethyl-[2,3′-bipyridin]-5′-yl)acetate (12 mg, 0.020 mmol, 8.33% yield) as a red/brown oil. ESI-MS(+) m/z=606.4 (M+1).

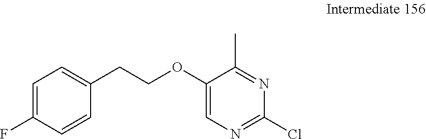 [SMILES: Cc1nc(Cl)ncc1OCCc1ccc(F)cc1]

### 2-Chloro-5-(4-fluorophenethoxy)-4-methylpyrimidine

To a 50 mL round bottom flask equipped with a stir bar was added 2-chloro-4-methylpyrimidin-5-ol (250 mg, 1.729 mmol), 2-(4-fluorophenyl)ethan-1-ol (242 mg, 1.729 mmol), triphenylphosphine (544 mg, 2.075 mmol) and THF (10 mL). To the stirred solution was added DIAD (0.404 mL, 2.075 mmol). The solution warmed to a mild reflux, then cooled within 5 minutes. The solution was stirred at r.t. for 2 hrs. The reaction solution was concentrated in vacuo and the resulting oil was diluted with a min of acetone, then concentrated onto Celite in vacuo. The resulting powder was subjected to silica gel chromatography (40 g column, 5-40% EtOAc:Hex) to afford the product 2-chloro-5-(4-fluorophenethoxy)-4-methylpyrimidine (448 mg, 1.680 mmol, 97% yield) as a white solid. ¹H NMR (500 MHz, chloroform-d) δ 8.04 (s, 1H), 7.26 (dd, J=8.7, 5.4 Hz, 2H), 7.07-6.97 (m, 2H), 4.25 (t, J=6.5 Hz, 2H), 3.14 (t, J=6.5 Hz, 2H). ESI-MS(+) m/z=267 (M+1).

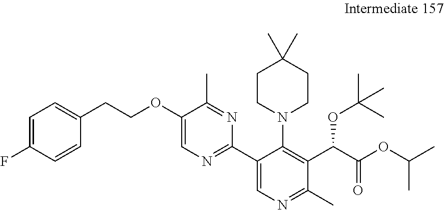 [SMILES: Cc1nc(-c2cnc(C)c([C@H](OC(C)(C)C)C(=O)OC(C)C)c2N2CCC(C)(C)CC2)ncc1OCCc1ccc(F)cc1]

### Isopropyl (S)-2-(tert-butoxy)-2-(4-(4, 4-dimethylpiperidin-1-yl)-5-(5-(4-fluorophenethoxy)-4-methylpyrimidin-2-yl)-2-methylpyridin-3-yl)acetate

To a 14 mL test tube equipped with a stir bar and added (S)-(5-(1-(tert-butoxy)-2-isopropoxy-2-oxoethyl)-4-(4,4-dimethylpiperidin-1-yl)-6-methylpyridin-3-yl)boronic acid (105 mg, 0.250 mmol) and SPhos-Pd-G3 (9.73 mg, 0.012 mmol), tribasic potassium phosphate (477 mg, 2.248 mmol) and 2-chloro-5-(4-fluorophenethoxy)-4-methylpyrimidine (66.6 mg, 0.250 mmol). The flask was sealed with a rubber septum, then was placed under N₂ atm (vac/fill×3). To the flask was added degassed (N₂ bubbling for 5 min) dioxane (937 μl) and water (312 μl). The test tube was placed in a 60° C. heating block with stirring (t=0). The reaction was stirred for 3 hrs. LCMS analysis at t=3 h found a large peak that corresponded to the expected product mass. The LCMS did not show any more boronic acid. The reaction was cooled to RT and diluted with EtOAc and water. The organic layer was washed with brine, collected, dried over MgSO₄, filtered and the volatiles evaporated to afford the crude product. The crude product was purified via silica gel (24 g column, 20-100% EtOAc:Hex), desired fractions collected and the volatiles evaporated to afford the product isopropyl (S)-2-(tert-butoxy)-2-(4-(4,4-dimethylpiperidin-1-yl)-5-(5-(4-fluorophenethoxy)-4-methylpyrimidin-2-yl)-2-methylpyridin-3-yl)acetate (26 mg, 0.043 mmol, 17.15% yield) as a brown oil. ESI-MS(+) m/z=607.7 (M+1).

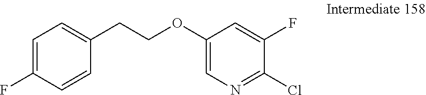 [SMILES: Fc1ccc(CCOc2cnc(Cl)c(F)c2)cc1]

### 2-Chloro-3-fluoro-5-(4-fluorophenethoxy)pyridine

To a 50 mL round bottom flask equipped with a stir bar was added 6-chloro-5-fluoropyridin-3-ol (250 mg, 1.695 mmol), 2-(4-fluorophenyl)ethan-1-ol (238 mg, 1.695 mmol), triphenylphosphine (533 mg, 2.033 mmol) and THF (10 mL). To the stirred solution was added DIAD (0.395 mL, 2.033 mmol). The solution warmed to a mild reflux, then cooled within 5 minutes. The solution was stirred at RT for 2 hrs. The reaction solution was concentrated in vacuo and the resulting oil was diluted with a min of acetone, then concentrated onto Celite in vacuo. The resulting powder was subjected to silica gel chromatography (40 g column, 5-40% EtOAc:Hex) to afford the product 2-chloro-3-fluoro-5-(4-fluorophenethoxy)pyridine (443 mg, 1.643 mmol, 97% yield) as a white solid. ¹H NMR (500 MHz, chloroform-d) δ 7.93 (d, J=2.5 Hz, 1H), 7.25 (dd, J=8.5, 5.4 Hz, 2H), 7.08-7.01 (m, 3H), 4.21 (t, J=6.7 Hz, 2H), 3.11 (t, J=6.8 Hz, 2H). ESI-MS(+) m/z=270 (M+1).

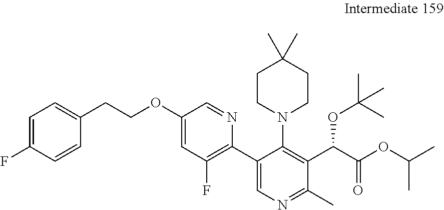 [SMILES: Cc1ncc(-c2ncc(OCCc3ccc(F)cc3)cc2F)c(N2CCC(C)(C)CC2)c1[C@H](OC(C)(C)C)C(=O)OC(C)C]

### Isopropyl (S)-2-(tert-butoxy)-2-(4′-(4, 4-dimethylpiperidin-1-yl)-3-fluoro-5-(4-fluorophenethoxy)-6′-methyl-[2, 3′-bipyridin]-5′-yl)acetate

To a 14 mL test tube equipped with a stir bar and (S)-(5-(1-(tert-butoxy)-2-isopropoxy-2-oxoethyl)-4-(4,4-dimethylpiperidin-1-yl)-6-methylpyridin-3-yl)boronic acid (100 mg, 0.238 mmol) was added SPhos-Pd-G3 (9.27 mg, 0.012 mmol), tribasic potassium phosphate (454 mg, 2.141 mmol) and 2-chloro-3-fluoro-5-(4-fluorophenethoxy)pyridine (64.2 mg, 0.238 mmol). The flask was sealed with a rubber septum, then was placed under N₂ atm (vac/fill×3). To the flask was added degassed (N₂ bubbling for 5 min) dioxane (892 μl) and water (297 μl). The test tube was placed in a 60° C. heating block with stirring (t=0). LCMS analysis at t=3 h found a large product mass peak and the disappearance of the starting boronic acid. The reaction was cooled to RT and diluted with EtOAc and water. The organic layer was washed with brine, collected, dried over MgSO₄, filtered and volatiles evaporated to afford the crude product. The crude product was purified via silica gel chromatography (24 g column, 20-100% EtOAc:Hex) to afford the product isopropyl (S)-2-(tert-butoxy)-2-(4′-(4,4-dimethylpiperidin-1-yl)-3-fluoro-5-(4-fluorophenethoxy)-6′-methyl-[2,3′-bipyridin]-5′-yl)acetate (24 mg, 0.039 mmol, 16.55% yield) as a red/brown oil. ESI-MS(+) m/z=610.3 (M+1).

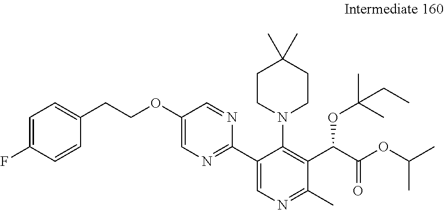 [SMILES: CCC(C)(C)O[C@H](C(=O)OC(C)C)c1c(C)ncc(-c2ncc(OCCc3ccc(F)cc3)cn2)c1N1CCC(C)(C)CC1]

### Isopropyl (S)-2-(4-(4, 4-dimethylpiperidin-1-yl)-5-(5-(4-fluorophenethoxy)pyrimidin-2-yl)-2-methylpyridin-3-yl)-2-(tert-pentyloxy)acetate

To a 14 mL test tube equipped with a stir bar and added (S)-(4-(4,4-dimethylpiperidin-1-yl)-5-(2-isopropoxy-2-oxo-1-(tert-pentyloxy)ethyl)-6-methylpyridin-3-yl)boronic acid (105 mg, 0.242 mmol) and SPhos-Pd-G3 (9.42 mg, 0.012 mmol), tribasic potassium phosphate (462 mg, 2.175 mmol) and 2-chloro-5-(4-fluorophenethoxy)pyrimidine (61.1 mg, 0.242 mmol). The flask was sealed with a rubber septum, then was placed under N₂ atm (vac/fill×3). To the flask was added degassed (N₂ bubbling for 5 min) dioxane (906 μl) and water (302 μl). The test tube was placed in a 60° C. heating block with stirring (t=0). The reaction was stirred for 3 hrs. The reaction was cooled to RT and diluted with EtOAc and water. The organic layer was washed with brine, collected, dried over MgSO₄, filtered and the volatiles evaporated to afford the crude product. The crude product was purified via silica gel (24 g column, 20-100% EtOAc:Hex) to afford the product isopropyl (S)-2-(4-(4,4-dimethylpiperidin-1-yl)-5-(5-(4-fluorophenethoxy)pyrimidin-2-yl)-2-methylpyridin-3-yl)-2-(tert-pentyloxy)acetate (21 mg, 0.035 mmol, 14.32% yield) as a brown oil. ESI-MS(+) m/z=607.7 (M+1).

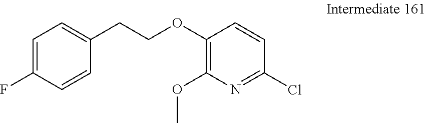 [SMILES: COc1nc(Cl)ccc1OCCc1ccc(F)cc1]

### 6-chloro-3-(4-fluorophenethoxy)-2-methoxypyridine

To a 50 mL round bottom flask equipped with a stir bar was added 6-chloro-2-methoxypyridin-3-ol (250 mg, 1.567 mmol), 2-(4-fluorophenyl)ethan-1-ol (220 mg, 1.567 mmol), triphenylphosphine (493 mg, 1.880 mmol) and THF (10 mL). To the stirred solution was added DIAD (0.366 mL, 1.880 mmol). The solution warmed to a mild reflux, then cooled within 5 minutes. The solution was stirred at r.t. for 2 hrs. The reaction solution was concentrated in vacuo and the resulting oil was diluted with a min of acetone, then concentrated onto Celite in vacuo. The resulting residue was subjected to silica gel chromatography (40 g column, 5-40% EtOAc:Hex) to afford the product 6-chloro-3-(4-fluorophenethoxy)-2-methoxypyridine (378 mg, 1.342 mmol, 86% yield) as a white solid. ¹H NMR (500 MHz, chloroform-d) δ 7.26 (dd, J=8.5, 5.5 Hz, 2H), 7.06-6.97 (m, 3H), 6.83 (d, J=8.2 Hz, 1H), 4.17 (t, J=7.2 Hz, 2H), 3.13 (t, J=7.2 Hz, 2H). ESI-MS(+) m/z=282 (M+1).

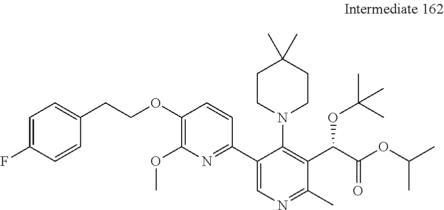 [SMILES: COc1nc(-c2cnc(C)c([C@H](OC(C)(C)C)C(=O)OC(C)C)c2N2CCC(C)(C)CC2)ccc1OCCc1ccc(F)cc1]

### (S)-2-(tert-Butoxy)-2-(4′-(4, 4-dimethylpiperidin-1-yl)-5-(4-fluorophenethoxy)-6-methoxy-6′-methyl-[2, 3′-bipyridin]-5′-yl)acetate

To a 14 mL test tube equipped with a stir bar was added (S)-(5-(1-(tert-butoxy)-2-isopropoxy-2-oxoethyl)-4-(4,4-dimethylpiperidin-1-yl)-6-methylpyridin-3-yl)boronic acid (105 mg, 0.250 mmol), SPhos-Pd-G3 (9.73 mg, 0.012 mmol), tribasic potassium phosphate (477 mg, 2.248 mmol) and 6-chloro-3-(4-fluorophenethoxy)-2-methoxypyridine (70.4 mg, 0.250 mmol). The flask was sealed with a rubber septum, then was placed under N₂ atm (vac/fill×3). To the flask was added degassed (N₂ bubbling for 5 min) dioxane (937 μl) and water (312 μl). The test tube was placed in a 60° C. heating block with stirring (t=0). LCMS analysis at t=3 h found a large product mass peak and the disappearance of the starting boronic acid. The reaction was cooled to RT and diluted with EtOAc and water. The organic layer was washed with brine, collected, dried over MgSO₄, filtered and volatiles evaporated to afford the crude product. The crude product was purified via silica gel chromatography (24 g column, 20-100% EtOAc:Hex) to afford the product isopropyl (S)-2-(tert-butoxy)-2-(4′-(4,4-dimethylpiperidin-1-yl)-5-(4-fluorophenethoxy)-6-methoxy-6′-methyl-[2,3′-bipyridin]-5′-yl)acetate (77 mg, 0.124 mmol, 49.6% yield) as a brown oil. ESI-MS(+) m/z=622.3 (M+1).

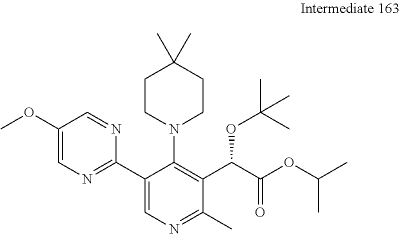 [SMILES: COc1cnc(-c2cnc(C)c([C@H](OC(C)(C)C)C(=O)OC(C)C)c2N2CCC(C)(C)CC2)nc1]

### (S)-Isopropyl 2-(tert-butoxy)-2-(4-(4, 4-dimethylpiperidin-1-yl)-5-(5-methoxypyrimidin-2-yl)-2-methylpyridin-3-yl)acetate

To a 14 mL test tube equipped with a stir bar was added (S)-(5-(1-(tert-butoxy)-2-isopropoxy-2-oxoethyl)-4-(4,4-dimethylpiperidin-1-yl)-6-methylpyridin-3-yl)boronic acid (113 mg, 0.269 mmol) and SPhos-Pd-G3 (10.47 mg, 0.013 mmol), tribasic potassium phosphate (514 mg, 2.419 mmol) and 2-chloro-5-methoxypyrimidine (38.9 mg, 0.269 mmol). The flask was sealed with a rubber septum, then was placed under N₂ atm (vac/fill×3). To the flask was added degassed (N₂ bubbling for 5 min) dioxane (1008 μl) and water (336 μl). The test tube was placed in a 60° C. heating block with stirring (t=0). The reaction was stirred for 3 hrs. The reaction was cooled to RT and diluted with EtOAc and water. The organic layer was washed with brine, collected, dried over MgSO₄, filtered and the volatiles evaporated to afford the crude product. The crude product was purified on silica gel (24 g column, 20-100% EtOAc:Hex) to afford the product (S)-isopropyl 2-(tert-butoxy)-2-(4-(4,4-dimethylpiperidin-1-yl)-5-(5-methoxypyrimidin-2-yl)-2-methylpyridin-3-yl)acetate (26 mg, 0.054 mmol, 19.96% yield) as a brown oil. ESI-MS(+) m/z=485.3 (M+1).

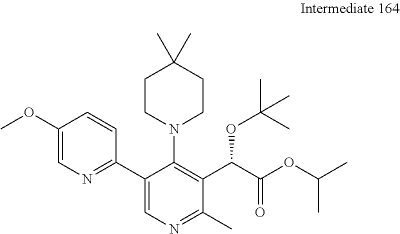 [SMILES: COc1ccc(-c2cnc(C)c([C@H](OC(C)(C)C)C(=O)OC(C)C)c2N2CCC(C)(C)CC2)nc1]

### (S)-2-(tert-Butoxy)-2-(4′-(4, 4-dimethylpiperidin-1-yl)-5-methoxy-6′-methyl-[2, 3′-bipyridin]-5′-yl)acetate

To a 14 mL test tube equipped with a stir bar was added (S)-(5-(1-(tert-butoxy)-2-isopropoxy-2-oxoethyl)-4-(4,4-dimethylpiperidin-1-yl)-6-methylpyridin-3-yl)boronic acid (105 mg, 0.250 mmol), SPhos-Pd-G3 (9.73 mg, 0.012 mmol), tribasic potassium phosphate (477 mg, 2.248 mmol) and 2-chloro-5-methoxypyridine (35.9 mg, 0.250 mmol). The flask was sealed with a rubber septum, then was placed under N₂ atm (vac/fill×3). To the flask was added degassed (N₂ bubbling for 5 min) dioxane (937 μl) and water (312 μl). The test tube was placed in a 60° C. heating block with stirring (t=0). The reaction was stirred for 3 hours. The reaction was cooled to RT and diluted with water and EtOAc. The organic layer was washed with brine, collected, dried over MgSO₄, filtered and the volatiles evaporated to afford the crude product. The crude product was purified on silica gel (24 g column, 20-100% EtOAc:Hex) to afford the product isopropyl (S)-2-(tert-butoxy)-2-(4′-(4,4-dimethylpiperidin-1-yl)-5-methoxy-6′-methyl-[2,3′-bipyridin]-5′-yl)acetate (22 mg, 0.045 mmol, 18.21% yield) as a brown oil. ESI-MS(+) m/z=484.3 (M+1).

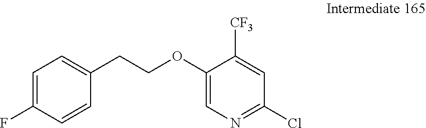 [SMILES: Fc1ccc(CCOc2cnc(Cl)cc2C(F)(F)F)cc1]

### 2-Chloro-5-(4-fluorophenethoxy)-4-(trifluoromethyl)pyridine

To a 40 mL vial equipped with a stir bar was added 6-chloro-4-(trifluoromethyl)pyridin-3-ol (1.00 g, 5.06 mmol), 2-(4-fluorophenyl)ethan-1-ol (0.710 g, 5.06 mmol), triphenylphosphine (1.593 g, 6.07 mmol) and THF (25 mL). To the stirred solution was added DIAD (1.181 mL, 6.07 mmol). The solution warmed to a mild reflux, then cooled within 5 minutes. The solution was stirred at r.t. (t=0). LCMS analysis at t=24 h found a major peak corresponding to the desired product mass. The reaction solution was concentrated in vacuo. The resulting residue was dissolved in a min of acetone, then was concentrated onto Celite in vacuo. The resulting powder was subjected to SiO2 purification on the Interchim system using ESI detection. The column conditions were 80 g SiO₂ column, hexanes:EtOAc 100:0->80:20 over 16 CV to afford the product 2-chloro-5-(4-fluorophenethoxy)-4-(trifluoromethyl)pyridine (1.3675 g, 4.28 mmol, 85% yield) as a colorless liquid. ¹H NMR (500 MHz, chloroform-d) δ 8.14 (s, 1H), 7.46 (s, 1H), 7.26-7.21 (m, 2H), 7.04-6.96 (m, 2H), 4.31 (t, J=6.4 Hz, 2H).

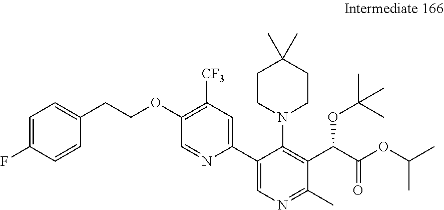 [SMILES: Cc1ncc(-c2cc(C(F)(F)F)c(OCCc3ccc(F)cc3)cn2)c(N2CCC(C)(C)CC2)c1[C@H](OC(C)(C)C)C(=O)OC(C)C]

### Isopropyl (S)-2-(tert-butoxy)-2-(4′-(4, 4-dimethylpiperidin-1-yl)-5-(4-fluorophenethoxy)-6′-methyl-4-(trifluoromethyl)-[2, 3′-bipyridin]-5′-yl)acetate

To a 14 mL test tube equipped with a stir bar and (S)-(5-(1-(tert-butoxy)-2-isopropoxy-2-oxoethyl)-4-(4,4-dimethylpiperidin-1-yl)-6-methylpyridin-3-yl)boronic acid (100 mg, 0.238 mmol) was added SPhos-Pd-G3 (9.27 mg, 0.012 mmol), tribasic potassium phosphate (454 mg, 2.141 mmol) and 2-chloro-5-(4-fluorophenethoxy)-4-(trifluoromethyl)pyridine (76 mg, 0.238 mmol). The flask was sealed with a rubber septum, then was placed under N₂ atm (vac/fill×3). To the flask was added degassed (N₂ bubbling for 5 min) dioxane (892 μl) and water (297 μl). The test tube was placed in a 60° C. heating block with stirring for t=3 h. The reaction was cooled to RT and diluted with EtOAc and water. The organic layer was washed with brine, collected, dried over MgSO₄, filtered and volatiles evaporated to afford the crude product. The crude product was purified via silica gel chromatography (24 g column, 20-100% EtOAc:Hex) to afford the product isopropyl (S)-2-(tert-butoxy)-2-(4′-(4,4-dimethylpiperidin-1-yl)-5-(4-fluorophenethoxy)-6′-methyl-4-(trifluoromethyl)-[2,3′-bipyridin]-5′-yl)acetate (36 mg, 0.055 mmol, 22.94% yield) as a red/brown oil. ESI-MS(+) m/z=660.3 (M+1).

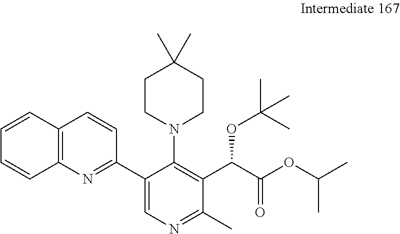 [SMILES: Cc1ncc(-c2ccc3ccccc3n2)c(N2CCC(C)(C)CC2)c1[C@H](OC(C)(C)C)C(=O)OC(C)C]

### Isopropyl (S)-2-(tert-butoxy)-2-(4-(4, 4-dimethylpiperidin-1-yl)-2-methyl-5-(quinolin-2-yl)pyridin-3-yl)acetate

To a 14 mL test tube equipped with a stir bar and added (S)-(5-(1-(tert-butoxy)-2-isopropoxy-2-oxoethyl)-4-(4,4-dimethylpiperidin-1-yl)-6-methylpyridin-3-yl)boronic acid (105 mg, 0.250 mmol) and SPhos-Pd-G3 (9.73 mg, 0.012 mmol), tribasic potassium phosphate (477 mg, 2.248 mmol) and 2-chloroquinoline (40.9 mg, 0.250 mmol). The flask was sealed with a rubber septum, then was placed under N₂ atm (vac/fill×3). To the flask was added degassed (N₂ bubbling for 5 min) dioxane (937 μl) and water (312 μl). The test tube was placed in a 60° C. heating block with stirring (t=0). The reaction was stirred for 3 hrs. The reaction was cooled to RT and diluted with EtOAc and water. The organic layer was washed with brine, collected, dried over MgSO₄, filtered and the volatiles evaporated to afford the crude product. The crude product was purified silica gel chromatography (24 g column, 20-100% EtOAc:Hex) to afford the product isopropyl (S)-2-(tert-butoxy)-2-(4-(4,4-dimethylpiperidin-1-yl)-2-methyl-5-(quinolin-2-yl)pyridin-3-yl)acetate (74 mg, 0.147 mmol, 58.8% yield) as a brown oil. ESI-MS(+) m/z=504.3 (M+1).

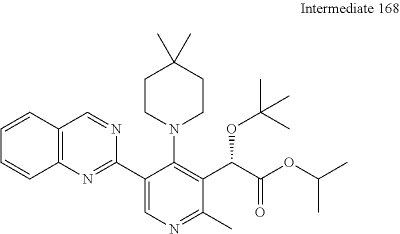 [SMILES: Cc1ncc(-c2ncc3ccccc3n2)c(N2CCC(C)(C)CC2)c1[C@H](OC(C)(C)C)C(=O)OC(C)C]

### Isopropyl (S)-2-(tert-butoxy)-2-(4-(4, 4-dimethylpiperidin-1-yl)-2-methyl-5-(quinazolin-2-yl)pyridin-3-yl)acetate

To a 14 mL test tube equipped with a stir bar and added (S)-(5-(1-(tert-butoxy)-2-isopropoxy-2-oxoethyl)-4-(4,4-dimethylpiperidin-1-yl)-6-methylpyridin-3-yl)boronic acid (105 mg, 0.250 mmol) and SPhos-Pd-G3 (9.73 mg, 0.012 mmol), tribasic potassium phosphate (477 mg, 2.248 mmol) and 2-chloroquinazoline (41.1 mg, 0.250 mmol). The flask was sealed with a rubber septum, then was placed under N₂ atm (vac/fill×3). To the flask was added degassed (N₂ bubbling for 5 min) dioxane (937 μl) and water (312.l).

The test tube was placed in a 60° C. heating block with stirring (t=0). The reaction was stirred for 3 hrs. The reaction was cooled to RT and diluted with EtOAc and water. The organic layer was washed with brine, collected, dried over MgSO₄, filtered and the volatiles evaporated to afford the crude product. The crude product was purified silica gel chromatography (24 g column, 20-100% EtOAc:Hex) to afford the product isopropyl (S)-2-(tert-butoxy)-2-(4-(4,4-dimethylpiperidin-1-yl)-2-methyl-5-(quinazolin-2-yl)pyridin-3-yl)acetate (13 mg, 0.026 mmol, 10.31% yield) as a brown oil. ESI-MS(+) m/z=505.3 (M+1).

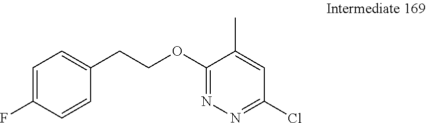 [SMILES: Cc1cc(Cl)nnc1OCCc1ccc(F)cc1]

### 6-Chloro-3-(4-fluorophenethoxy)-4-methylpyridazine

To a 50 mL round bottom flask equipped with a stir bar was added 6-chloro-4-methylpyridazin-3-ol (250 mg, 1.729 mmol), 2-(4-fluorophenyl)ethan-1-ol (0.216 mL, 1.729 mmol), triphenylphosphine (544 mg, 2.075 mmol) and THF (10 mL). To the stirred solution was added DIAD (0.404 mL, 2.075 mmol). The solution warmed to a mild reflux, then cooled within 5 minutes. The solution was stirred at r.t. for 2 hrs. The reaction solution was concentrated in vacuo and the resulting oil was diluted with a min of acetone, then concentrated onto Celite in vacuo. The resulting powder was subjected to silica gel chromatography (40 g column, 5-40% EtOAc:Hex) to afford the product 6-chloro-3-(4-fluorophenethoxy)-4-methylpyridazine (429 mg, 1.609 mmol, 93% yield) as a white solid. ¹H NMR (500 MHz, chloroform-d) δ 7.25-7.20 (m, 2H), 7.08 (d, J=1.3 Hz, 1H), 7.01 (t, J=8.7 Hz, 2H), 4.35-4.30 (m, 2H), 3.12-3.06 (m, 2H), 2.23 (d, J=1.3 Hz, 3H). ESI-MS(+) m/z=267 (M+1).

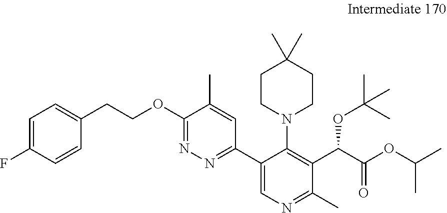 [SMILES: Cc1cc(-c2cnc(C)c([C@H](OC(C)(C)C)C(=O)OC(C)C)c2N2CCC(C)(C)CC2)nnc1OCCc1ccc(F)cc1]

### Isopropyl (S)-2-(tert-butoxy)-2-(4-(4, 4-dimethylpiperidin-1-yl)-5-(6-(4-fluorophenethoxy)-5-methylpyridazin-3-yl)-2-methylpyridin-3-yl)acetate

To a 14 mL test tube equipped with a stir bar and added (S)-(5-(1-(tert-butoxy)-2-isopropoxy-2-oxoethyl)-4-(4,4-dimethylpiperidin-1-yl)-6-methylpyridin-3-yl)boronic acid (105 mg, 0.250 mmol) and SPhos-Pd-G3 (9.73 mg, 0.012 mmol), tribasic potassium phosphate (477 mg, 2.248 mmol) and 6-chloro-3-(4-fluorophenethoxy)-4-methylpyridazine (66.6 mg, 0.250 mmol). The flask was sealed with a rubber septum, then was placed under N₂ atm (vac/fill×3). To the flask was added degassed (N₂ bubbling for 5 min) dioxane (937 μl) and water (312 μl). The test tube was placed in a 60° C. heating block with stirring (t=0). The reaction was stirred for 3 hrs. The reaction was cooled to RT and diluted with EtOAc and water. The organic layer was washed with brine, collected, dried over MgSO₄, filtered and the volatiles evaporated to afford the crude product. The crude product was purified via silica gel (24 g column, 20-100% EtOAc:Hex), desired fractions collected and the volatiles evaporated to afford the product isopropyl (S)-2-(tert-butoxy)-2-(4-(4,4-dimethylpiperidin-1-yl)-5-(6-(4-fluorophenethoxy)-5-methylpyridazin-3-yl)-2-methylpyridin-3-yl)acetate (56 mg, 0.092 mmol, 36.9% yield) as a brown oil. ESI-MS(+) m/z=607.4 (M+1).

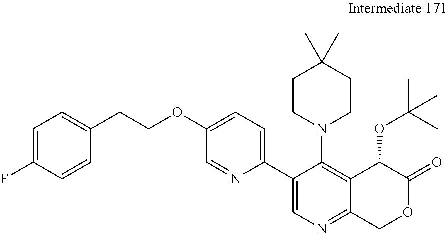 [SMILES: CC1(C)CCN(c2c(-c3ccc(OCCc4ccc(F)cc4)cn3)cnc3c2[C@H](OC(C)(C)C)C(=O)OC3)CC1]

### (S)-5-(tert-Butoxy)-4-(4, 4-dimethylpiperidin-1-yl)-3-(5-(4-fluorophenethoxy)pyridin-2-yl)-5, 8-dihydro-6H-pyrano[3, 4-b]pyridin-6-one

To a dry 10 mL Schlenk flask equipped with a stir bar was added 2-(5-(4-fluorophenethoxy)pyridin-2-yl)-6-methyl-1,3,6,2-dioxazaborocane-4,8-dione (53.7 mg, 0.144 mmol), benzyl (S)-2-(5-bromo-4-(4,4-dimethylpiperidin-1-yl)-2-(hydroxymethyl)pyridin-3-yl)-2-(tert-butoxy)acetate (50 mg, 0.096 mmol), palladium tetrakis (22.25 mg, 0.019 mmol), diacetoxycopper (8.74 mg, 0.048 mmol) and anhydrous tribasic potassium phosphate, finely ground (102 mg, 0.481 mmol). The flask was sealed with a rubber septum, then placed under N₂ atm (vac/fill×3). To the flask was added a degassed (N₂ sparging for 5 min) solution of DMF+diethanolamine (10.12 mg, 0.096 mmol). The flask was placed in a 100° C. oil bath with stirring for 18 hrs. The reaction mixture was transferred to a 125 mL separatory funnel and was diluted with water:brine (1:1, 50 mL). The mixture was extracted with EtOAc (3×50 mL). The combined organics were washed with water:brine (1:1, 50 mL), then brine (50 mL). The organics were dried over MgSO₄; filtered; then concentrated in vacuo to afford an amber oil. This material was dissolved in a min of acetone, then was concentrated onto Celite in vacuo. The resulting powder was subjected to SiO2 purification (24 g column, 5-100% EtOAc:Hex) to afford the exclusive product (S)-5-(tert-butoxy)-4-(4,4-dimethylpiperidin-1-yl)-3-(5-(4-fluorophenethoxy)pyridin-2-yl)-5,8-dihydro-6H-pyrano[3,4-b]pyridin-6-one (10.1 mg, 0.018 mmol, 19.16% yield) as a light yellow oil. ¹H NMR (500 MHz, methanol-d4) δ 8.13 (d, J=2.4 Hz, 1H), 8.04 (s, 1H), 7.37-7.31 (m, 1H), 7.29-7.23 (m, 1H), 7.16 (dd, J=8.1, 5.6 Hz, 2H), 6.85 (t, J=8.7 Hz, 2H), 5.70 (d, J=14.0 Hz, 1H), 5.17 (s, 1H), 4.94 (d, J=14.0 Hz, 1H), 4.15 (t, J=6.6 Hz, 2H), 2.94 (t, J=6.6 Hz, 2H), 2.74-2.66 (m, 2H), 2.61-2.50 (m, 2H), 1.20 (br t, J=5.2 Hz, 4H), 1.16 (s, 9H), 0.72 (s, 6H). ESI-MS(+) m/z=548.3 (M+1).

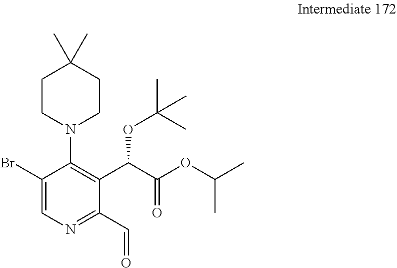 [SMILES: CC(C)OC(=O)[C@@H](OC(C)(C)C)c1c(C=O)ncc(Br)c1N1CCC(C)(C)CC1]

### (S)-Isopropyl 2-(5-bromo-4-(4, 4-dimethylpiperidin-1-yl)-2-formylpyridin-3-yl)-2-(tert-butoxy)acetate

To a stirred solution of (S)-isopropyl 2-(5-bromo-4-(4,4-dimethylpiperidin-1-yl)-2-(hydroxymethyl)pyridin-3-yl)-2-(tert-butoxy)acetate (0.443 g, 0.940 mmol) in DCM (8.54 ml) and Acetonitrile (0.854 ml) was added Dess-MartinPeriodinane (0.598 g, 1.410 mmol) at once at rt. After 6 h, the reaction mixture was diluted with ether (25 mL), washed with 1M NaOH (2×25 ml), brine (25 mL), dried (MgSO₄), filtered and concentrated to afford the product (S)-isopropyl 2-(5-bromo-4-(4,4-dimethylpiperidin-1-yl)-2-formylpyridin-3-yl)-2-(tert-butoxy)acetate (425 mg, 0.905 mmol, 96% yield) as a yellow oil. ESI-MS(+) m/z=469.1 (M+1).

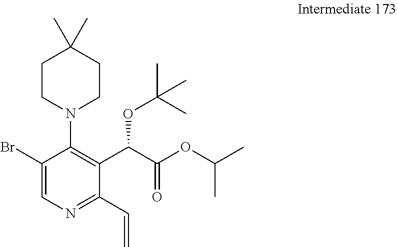 [SMILES: C=Cc1ncc(Br)c(N2CCC(C)(C)CC2)c1[C@H](OC(C)(C)C)C(=O)OC(C)C]

### (S)-Isopropyl 2-(5-bromo-4-(4,4-dimethylpiperidin-1-yl)-2-vinylpyridin-3-yl)-2-(tert-butoxy)acetate

To a suspension of methyltriphenylphosphonium bromide (753 mg, 2.109 mmol) in THF (8 ml) at 0° C. was added sodium hydride (84 mg, 2.109 mmol) and the resulting mixture was stirred at rt for 45 min. (S)-isopropyl 2-(5-bromo-4-(4,4-dimethylpiperidin-1-yl)-2-formylpyridin-3-yl)-2-(tert-butoxy)acetate (330 mg, 0.703 mmol) dissolved in THF (0.5 mL) THF (8 ml) was added dropwise and the mixture was stirred at 0° C. for 1 h then warmed to rt and stirred 18 h. The reaction was quenched with water and the product was extracted with EtOAc. The organic phase was washed with brine, dried (MgSO₄), filtered and concentrated. The residue was purified by silica gel chromatography (40 g column; 5-20% EtOAc/hexane) to afford (S)-isopropyl 2-(5-bromo-4-(4,4-dimethylpiperidin-1-yl)-2-vinylpyridin-3-yl)-2-(tert-butoxy)acetate (184 mg, 0.394 mmol, 56.0% yield). ESI-MS(+) m/z=467.3 (M+1).

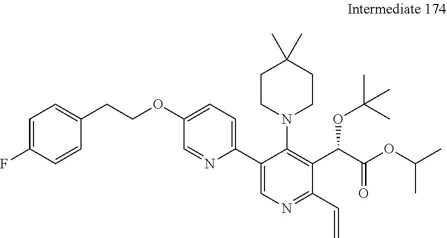 [SMILES: C=Cc1ncc(-c2ccc(OCCc3ccc(F)cc3)cn2)c(N2CCC(C)(C)CC2)c1[C@H](OC(C)(C)C)C(=O)OC(C)C]

### Isopropyl (S)-2-(tert-butoxy)-2-(4′-(4, 4-dimethylpiperidin-1-yl)-5-(4-fluorophenethoxy)-6′-vinyl-[2, 3′-bipyridin]-5′-yl)acetate

To a dry 50 mL Schlenk flask equipped with a stir bar was added 2-(5-(4-fluorophenethoxy)pyridin-2-yl)-6-methyl-1,3,6,2-dioxazaborocane-4,8-dione (119 mg, 0.321 mmol), isopropyl (S)-2-(5-bromo-4-(4,4-dimethylpiperidin-1-yl)-2-vinylpyridin-3-yl)-2-(tert-butoxy)acetate (100 mg, 0.214 mmol), palladium tetrakis (49.4 mg, 0.043 mmol), diacetoxycopper (19.43 mg, 0.107 mmol) and anhydrous tribasic potassium phosphate, finely ground (227 mg, 1.070 mmol). The flask was sealed with a rubber septum, then placed under N₂ atm (vac/fill×3). To the flask was added a degassed (N₂ sparging for 5 min) solution of DMF+diethanolamine (22.49 mg, 0.214 mmol). The flask was placed in a 100° C. oil bath with stirring (t=0) and stirred for 18 hrs. The reaction mixture was transferred to a 125 mL separatory funnel and was diluted with water:brine (1:1, 50 mL). The mixture was extracted with EtOAc (3×50 mL). The combined organics were washed with water:brine (1:1, 50 mL), then brine (50 mL). The organics were dried over MgSO₄; filtered; then concentrated in vacuo to afford an amber oil. This material was dissolved in a min of acetone, then was concentrated onto Celite in vacuo. The resulting powder was subjected to SiO2 purification to afford the the desired product isopropyl (S)-2-(tert-butoxy)-2-(4′-(4,4-dimethylpiperidin-1-yl)-5-(4-fluorophenethoxy)-6′-vinyl-[2,3′-bipyridin]-5′-yl)acetate (63 mg, 0.104 mmol, 48.8% yield). ¹H NMR (500 MHz, methanol-d4) δ 8.36 (d, J=2.5 Hz, 1H), 8.26 (s, 1H), 7.58-7.53 (m, 1H), 7.51-7.46 (m, 1H), 7.41-7.29 (m, 3H), 7.10-7.02 (m, 2H), 6.20 (dd, J=17.0, 2.2 Hz, 1H), 6.11 (br s, 1H), 5.42 (br d, J=12.1 Hz, 1H), 5.09 (dt, J=12.5, 6.3 Hz, 1H), 4.42-4.32 (m, 2H), 3.16 (t, J=6.5 Hz, 2H), 2.41-2.21 (m, 1H), 2.18-2.08 (m, 1H), 1.74-1.57 (m, 1H), 1.55-1.29 (m, 3H), 1.27-1.25 (m, 3H), 1.20 (s, 9H), 1.18 (d, J=6.1 Hz, 3H), 0.99-0.80 (m, 6H) 2 protons on the piperidine (closest to N) were not observed in HNMR. ESI-MS(+) m/z=604.4 (M+1).

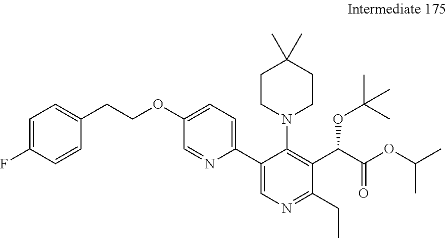 [SMILES: CCc1ncc(-c2ccc(OCCc3ccc(F)cc3)cn2)c(N2CCC(C)(C)CC2)c1[C@H](OC(C)(C)C)C(=O)OC(C)C]

### Isopropyl (S)-2-(tert-butoxy)-2-(4′-(4, 4-dimethylpiperidin-1-yl)-6′-ethyl-5-(4-fluorophenethoxy)-[2, 3′-bipyridin]-5′-yl)acetate

To an N₂ sparged solution of isopropyl (S)-2-(tert-butoxy)-2-(4′-(4,4-dimethylpiperidin-1-yl)-5-(4-fluorophenethoxy)-6′-vinyl-[2,3′-bipyridin]-5′-yl)acetate (63 mg, 0.104 mmol) in MeOH (2.5 mL) was added Pd/C (11.10 mg, 10.43 μmol) and capped with a rubber septum. H₂ was then bubbled through the solution for 10 minutes. The reaction was left under positive pressure of H₂ for 1 hr. The LCMS indicated the reaction was complete. The reaction was filtered through a 0.45 t nylon frit filter and the volatiles evaporated to afford the product isopropyl (S)-2-(tert-butoxy)-2-(4′-(4,4-dimethylpiperidin-1-yl)-6′-ethyl-5-(4-fluorophenethoxy)-[2,3′-bipyridin]-5′-yl)acetate (63 mg, 0.104 mmol, 100% yield) as a clear oil. ESI-MS(+) m/z=606.7 (M+1).

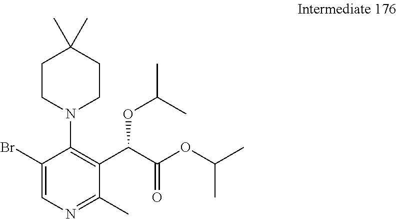 [SMILES: Cc1ncc(Br)c(N2CCC(C)(C)CC2)c1[C@H](OC(C)C)C(=O)OC(C)C]

### Isopropyl (S)-2-(5-bromo-4-(4, 4-dimethylpiperidin-1-yl)-2-methylpyridin-3-yl)-2-isopropoxyacetate

To a solution of isopropyl (S)-2-(5-bromo-4-(4,4-dimethylpiperidin-1-yl)-2-methylpyridin-3-yl)-2-hydroxyacetate (50 mg, 0.125 mmol) and 2-iodopropane (0.025 mL, 0.250 mmol) in DMF (1.5 mL) was added sodium hydride (10.02 mg, 0.250 mmol) and stirred at RT. The reaction was cooled to RT and 2-iodopropane (0.025 mL, 0.250 mmol) was added followed by sodium hydride (10.02 mg, 0.250 mmol) and heated to 60° C. This process was repeated 6 more times over 2 days. At this point the material appeared to show ester hydrolysis. The reaction was quenched with water and diluted with EtOAc. The organic layer was washed with water (2×), followed by brine, dried over MgSO₄, filtered and volatiles evaporated to afford the crude product. The crude product was purified on silica gel (12 g column, 5-40% EtOAc:Hex) to afford the product isopropyl (S)-2-(5-bromo-4-(4,4-dimethylpiperidin-1-yl)-2-methylpyridin-3-yl)-2-isopropoxyacetate (28 mg, 0.063 mmol, 50.7% yield) as a clear oil. ESI-MS(+) m/z=441.3 and 443.3.

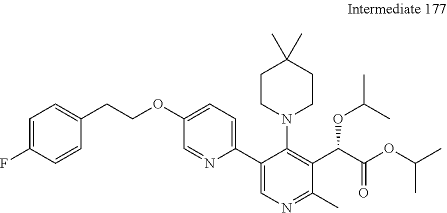 [SMILES: Cc1ncc(-c2ccc(OCCc3ccc(F)cc3)cn2)c(N2CCC(C)(C)CC2)c1[C@H](OC(C)C)C(=O)OC(C)C]

### Isopropyl (S)-2-(4′-(4, 4-dimethylpiperidin-1-yl)-5-(4-fluorophenethoxy)-6′-methyl-[2, 3′-bipyridin]-5′-yl)-2-isopropoxyacetate

To a dry 10 mL Schlenk flask equipped with a stir bar was added 2-(5-(4-fluorophenethoxy)pyridin-2-yl)-6-methyl-1,3,6,2-dioxazaborocane-4,8-dione (35.4 mg, 0.095 mmol), isopropyl (S)-2-(5-bromo-4-(4,4-dimethylpiperidin-1-yl)-2-methylpyridin-3-yl)-2-isopropoxyacetate (28 mg, 0.063 mmol), palladium tetrakis (14.66 mg, 0.013 mmol), diacetoxycopper (5.76 mg, 0.032 mmol) and anhydrous tribasic potassium phosphate, finely ground (67.3 mg, 0.317 mmol). The flask was sealed with a rubber septum, then placed under N₂ atm (vac/fill×3). To the flask was added a degassed (N₂ sparging for 5 min) solution of DMF+diethanolamine (6.67 mg, 0.063 mmol). The flask was placed in a 100° C. oil bath with stirring for t=18 h. The reaction mixture was transferred to a 125 mL separatory funnel and was diluted with water:brine (1:1, 50 mL). The mixture was extracted with EtOAc (3×50 mL). The combined organics were washed with water:brine (1:1, 50 mL), then brine (50 mL). The organics were dried over MgSO₄; filtered; then concentrated in vacuo to afford an amber oil. This material was dissolved in a min of acetone, then was concentrated onto Celite in vacuo. The resulting powder was subjected to SiO2 purification. The pure fractions were pooled and concentrated in vacuo to afford the product isopropyl (S)-2-(4′-(4,4-dimethylpiperidin-1-yl)-5-(4-fluorophenethoxy)-6′-methyl-[2,3′-bipyridin]-5′-yl)-2-isopropoxyacetate (15 mg, 0.026 mmol, 40.9% yield). ESI-MS(+) m/z=578.5 (M+1).

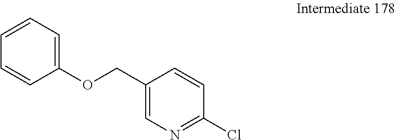 [SMILES: Clc1ccc(COc2ccccc2)cn1]

### 2-Chloro-5-(phenoxymethyl)pyridine

To a 50 mL round bottom flask equipped with a stir bar was added (6-chloropyridin-3-yl)methanol (250 mg, 1.741 mmol), phenol (164 mg, 1.741 mmol), triphenylphosphine (548 mg, 2.090 mmol) and THF (10 mL). To the stirred solution was added DIAD (0.406 mL, 2.090 mmol). The solution warmed to a mild reflux, then cooled within 5 minutes. The solution was stirred at r.t. for 2 hrs. The reaction solution was concentrated in vacuo and the resulting oil was diluted with a min of acetone, then concentrated onto Celite in vacuo. The resulting powder was subjected to silica gel chromatography (40 g column, 5-40% EtOAc:Hex) to afford the product 2-chloro-5-(phenoxymethyl)pyridine (305 mg, 1.388 mmol, 80% yield) as a white solid. ¹H NMR (500 MHz, chloroform-d) δ 8.48 (d, J=1.9 Hz, 1H), 7.78 (dd, J=8.2, 2.4 Hz, 1H), 7.39 (d, J=8.0 Hz, 1H), 7.36-7.31 (m, 2H), 7.29-7.24 (m, 1H), 7.03 (t, J=7.3 Hz, 1H), 6.89-6.84 (m, 1H), 5.09 (s, 2H).

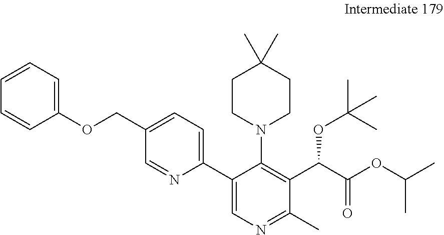 [SMILES: Cc1ncc(-c2ccc(COc3ccccc3)cn2)c(N2CCC(C)(C)CC2)c1[C@H](OC(C)(C)C)C(=O)OC(C)C]

### Isopropyl (S)-2-(tert-butoxy)-2-(4′-(4, 4-dimethylpiperidin-1-yl)-6′-methyl-5-(phenoxymethyl)-[2, 3′-bipyridin]-5′-yl)acetate

To a 14 mL test tube equipped with a stir bar and (S)-(5-(1-(tert-butoxy)-2-isopropoxy-2-oxoethyl)-4-(4,4-dimethylpiperidin-1-yl)-6-methylpyridin-3-yl)boronic acid (210 mg, 0.500 mmol) was added 2-chloro-5-(phenoxymethyl)pyridine (27.4 mg, 0.125 mmol), potassium phosphate tribasic (954 mg, 4.50 mmol) and SPhos-Pd-G3 (19.46 mg, 0.025 mmol). The flask was sealed with a rubber septum, then was placed under N₂ atm (vac/fill×3). To the flask was added degassed (N₂ bubbling for 5 min) dioxane (1873 μl) and water (624 μl). The test tube was placed in a 60° C. heating block with stirring t=18 h. The reaction was cooled to RT and diluted with EtOAc and water. The organic layer was washed with brine, collected, dried over MgSO₄, filtered and volatiles evaporated to afford the crude product. The crude product was purified via silica gel chromatography (24 g column, 20-100% EtOAc:Hex) to afford the product isopropyl (S)-2-(tert-butoxy)-2-(4′-(4,4-dimethylpiperidin-1-yl)-6′-methyl-5-(phenoxymethyl)-[2,3′-bipyridin]-5′-yl)acetate (39 mg, 0.070 mmol, 55.8% yield) as a red/brown oil. ESI-MS(+) m/z=560.5 (M+1).

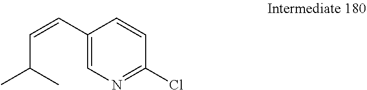

### (Z)-2-Chloro-5-(3-methylbut-1-en-1-yl)pyridine

To a 0° C. solution of isobutyltriphenylphosphonium bromide (705 mg, 1.766 mmol) in THF (10 mL) was added BuLi (0.706 mL, 1.766 mmol) dropwise and allowed to stir at 0° C. for 30 minutes. The reaction was then added 6-chloronicotinaldehyde (250 mg, 1.766 mmol) (in 2 mL THF) dropwise at 0° C. The reaction was then allowed to warm up to RT and stir for 2 hrs. The reaction was diluted with sat aq. ammonium chloride and extracted with EtOAc. The organic layer was washed with brine, collected, dried over MgSO₄, filtered and volatiles evaporated to afford the crude product. The crude product was purified on silica gel (24 g column, 5-20% EtOAc:Hex) to afford the product (Z)-2-chloro-5-(3-methylbut-1-en-1-yl)pyridine (240 mg, 1.321 mmol, 74.8% yield). ¹H NMR (500 MHz, chloroform-d) δ 8.30 (d, J=2.4 Hz, 1H), 7.54 (dd, J=8.2, 2.4 Hz, 1H), 7.30 (d, J=8.2 Hz, 1H), 6.22 (d, J=11.7 Hz, 1H), 5.66 (dd, J=11.5, 10.4 Hz, 1H), 2.78 (ddtd, J=13.1, 10.4, 6.6, 0.7 Hz, 1H), 1.07 (d, J=6.6 Hz, 6H).

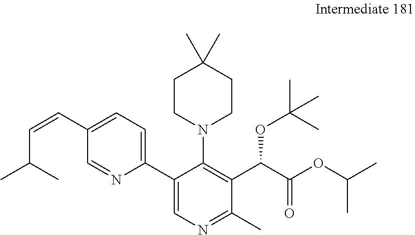 [SMILES: Cc1ncc(-c2ccc(/C=C\C(C)C)cn2)c(N2CCC(C)(C)CC2)c1[C@H](OC(C)(C)C)C(=O)OC(C)C]

### Isopropyl (S,Z)-2-(tert-butoxy)-2-(4′-(4, 4-dimethylpiperidin-1-yl)-6′-methyl-5-(3-methylbut-1-en-1-yl)-[2, 3′-bipyridin]-5′-yl)acetate

To a 14 mL test tube equipped with a stir bar and (S)-(5-(1-(tert-butoxy)-2-isopropoxy-2-oxoethyl)-4-(4,4-dimethylpiperidin-1-yl)-6-methylpyridin-3-yl)boronic acid (210 mg, 0.500 mmol) was added (Z)-2-chloro-5-(3-methylbut-1-en-1-yl)pyridine (22.69 mg, 0.125 mmol), potassium phosphate tribasic (954 mg, 4.50 mmol) and SPhos-Pd-G3 (19.46 mg, 0.025 mmol). The flask was sealed with a rubber septum, then was placed under N₂ atm (vac/fill×3). To the flask was added degassed (N₂ bubbling for 5 min) dioxane (1873 μl) and water (624 μl). The test tube was placed in a 60° C. heating block with stirring for t=18 h. The reaction was cooled to RT and diluted with EtOAc and water. The organic layer was washed with brine, collected, dried over MgSO₄, filtered and volatiles evaporated to afford the crude product. The crude product was purified via silica gel chromatography (24 g column, 20-100% EtOAc:Hex) to afford the impure product isopropyl (S,Z)-2-(tert-butoxy)-2-(4′-(4,4-dimethylpiperidin-1-yl)-6′-methyl-5-(3-methylbut-1-en-1-yl)-[2,3′-bipyridin]-5′-yl)acetate (164 mg, 0.314 mmol, 62.9% yield) as a red/brown oil. ESI-MS(+) m/z=522.3 (M+1).

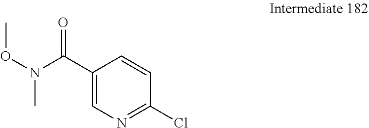

### 6-Chloro-N-methoxy-N-methylnicotinamide

To a solution of 6-chloronicotinic acid (2 g, 12.69 mmol) and N,O-dimethylhydroxylamine, HCl (1.362 g, 13.96 mmol) in DCM (50 mL) was added HATU (5.31 g, 13.96 mmol) followed by Hunig's Base (7.09 mL, 40.6 mmol) and stirred at RT for 18 hrs. The reaction was monitored via LCMS. After 18 hrs, the LCMS indicated that the reaction was complete. The organic phase was washed with water, followed by brine, dried over MgSO₄, filtered and volatiles evaporated to afford the crude product. The crude product was purified via silica gel (80 g column, 10-60% EtOAc:Hex) to afford the product 6-chloro-N-methoxy-N-methylnicotinamide (2.2 g, 10.97 mmol, 86% yield) as a clear thin oil. ¹H NMR (500 MHz, chloroform-d) δ 8.80 (d, J=1.9 Hz, 1H), 8.04 (dd, J=8.4, 2.4 Hz, 1H), 7.41 (dd, J=8.2, 0.6 Hz, 1H), 3.58 (s, 3H), 3.41 (s, 3H). ESI-MS(+) m/z=201.1 (M+1).

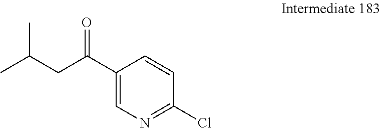

### 1-(6-Chloropyridin-3-yl)-3-methylbutan-1-one

To a 0° C. solution of 6-chloro-N-methoxy-N-methylnicotinamide (500 mg, 2.492 mmol) in THF (10 mL) was added dropwise isobutylmagnesium bromide (2.492 mL, 4.98 mmol) and allowed to stir for 15 min at 0° C. before allowing to warm up to RT. The reaction was then stirred for 1 hr. After stirring for 1 hr, the reaction was quenched with sat. aq. ammonium chloride and then extracted with EtOAc (50 mL). The organic layer was washed with brine, collected, dried over MgSO₄, filtered and volatiles evaporated to afford a yellow solid. The crude material was purified on silica gel (24 g column, 5-40% EtOAc:Hex) to afford the product 1-(6-chloropyridin-3-yl)-3-methylbutan-1-one (386 mg, 1.953 mmol, 78% yield) as a white solid. ¹H NMR (500 MHz, chloroform-d) δ 8.95 (d, J=2.4 Hz, 1H), 8.21 (dd, J=8.4, 2.5 Hz, 1H), 7.46 (dd, J=8.4, 0.6 Hz, 1H), 2.85 (d, J=6.9 Hz, 2H), 2.32 (dquin, J=13.4, 6.7 Hz, 1H), 1.03 (d, J=6.6 Hz, 6H).

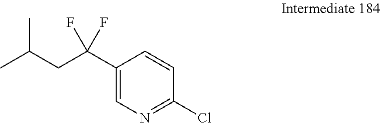

### 2-Chloro-5-(1,1-difluoro-3-methylbutyl)pyridine

Deoxofluor (3 mL, 8.14 mmol) (50% in toluene) was added dropwise to a solution of 1-(6-chloropyridin-3-yl)-3-methylbutan-1-one (386 mg, 1.953 mmol) in toluene (0.5 mL). The reaction was then heated to 60° C. and stirred for 18 hrs. The LCMS after 18 hrs, showed some product conversion. The reaction was cooled to RT and reverse added to an aq. 1.5 M K₃PO₄ solution and extracted with EtOAc. The organic layer was washed with brine, collected, dried over MgSO₄, filtered and volatiles evaporated to afford the crude product. The crude product was purified on silica gel (24 g column, 5-20% EtOAc:Hex) to afford the product as a clear oil. ¹H NMR (500 MHz, chloroform-d) δ 8.54 (d, J=1.7 Hz, 1H), 7.76 (dd, J=8.4, 2.5 Hz, 1H), 7.42 (d, J=8.4 Hz, 1H), 2.06 (td, J=17.6, 6.5 Hz, 2H), 1.88 (dt, J=13.2, 6.6 Hz, 1H), 1.00 (d, J=6.6 Hz, 6H).

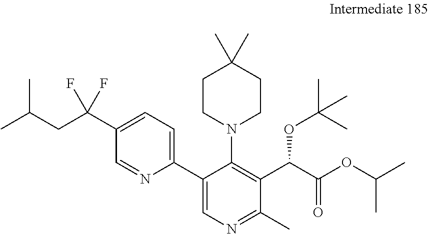 [SMILES: Cc1ncc(-c2ccc(C(F)(F)CC(C)C)cn2)c(N2CCC(C)(C)CC2)c1[C@H](OC(C)(C)C)C(=O)OC(C)C]

### Isopropyl (S)-2-(tert-butoxy)-2-(5-(1,1-difluoro-3-methylbutyl)-4′-(4, 4-dimethylpiperidin-1-yl)-6′-methyl-[2, 3′-bipyridin]-5′-yl)acetate

To a 14 mL test tube equipped with a stir bar and (S)-(5-(1-(tert-butoxy)-2-isopropoxy-2-oxoethyl)-4-(4,4-dimethylpiperidin-1-yl)-6-methylpyridin-3-yl)boronic acid (210 mg, 0.500 mmol) was added 2-chloro-5-(1,1-difluoro-3-methylbutyl)pyridine (27.4 mg, 0.125 mmol), potassium phosphate tribasic (954 mg, 4.50 mmol) and SPhos-Pd-G3 (19.46 mg, 0.025 mmol). The flask was sealed with a rubber septum, then was placed under N₂ atm (vac/fill×3). To the flask was added degassed (N₂ bubbling for 5 min) dioxane (1873 μl) and water (624 μl). The test tube was placed in a 60° C. heating block with stirring for t=18 h. The reaction was cooled to RT and diluted with EtOAc and water. The organic layer was washed with brine, collected, dried over MgSO₄, filtered and volatiles evaporated to afford the crude product. The crude product was purified via silica gel chromatography (24 g column, 20-100% EtOAc:Hex) to afford the product isopropyl (S)-2-(tert-butoxy)-2-(5-(1,1-difluoro-3-methylbutyl)-4′-(4,4-dimethylpiperidin-1-yl)-6′-methyl-[2,3′-bipyridin]-5′-yl)acetate (152 mg, 0.272 mmol, 54.4% yield) as a red/brown oil. ESI-MS(+) m/z=560.6 (M+1).

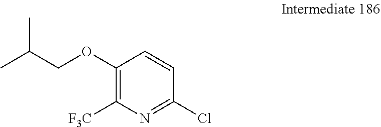 [SMILES: CC(C)COc1ccc(Cl)nc1C(F)(F)F]

### 6-Chloro-3-isobutoxy-2-(trifluoromethyl)pyridine

To a 50 mL round bottom flask equipped with a stir bar was added 6-chloro-2-(trifluoromethyl)pyridin-3-ol (250 mg, 1.266 mmol), 2-methylpropan-1-ol (0.234 mL, 1.266 mmol), triphenylphosphine (398 mg, 1.519 mmol) and THF (10 mL). To the stirred solution was added DIAD (0.295 mL, 1.519 mmol) dropwise. The solution was stirred at r.t. for 18 hrs. The reaction solution was concentrated in vacuo and the resulting oil was diluted with a min of acetone, then concentrated onto Celite in vacuo. The resulting powder was subjected to silica gel chromatography (24 g column, 0-20% EtOAc:Hex) to afford the product 6-chloro-3-isobutoxy-2-(trifluoromethyl)pyridine (225 mg, 0.887 mmol, 70.1% yield) as a clear oil. ¹H NMR (500 MHz, chloroform-d) δ 7.47 (d, J=8.8 Hz, 1H), 7.36 (d, J=8.8 Hz, 1H), 3.84 (d, J=6.3 Hz, 2H), 2.22-2.14 (m, 1H), 1.08 (d, J=6.6 Hz, 6H). ESI-MS(+) m/z=254.1 (M+1).

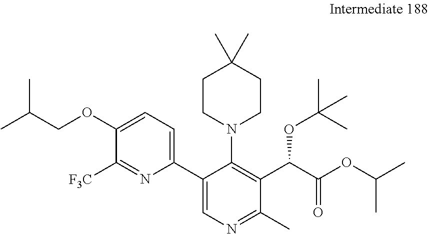 [SMILES: Cc1ncc(-c2ccc(OCC(C)C)c(C(F)(F)F)n2)c(N2CCC(C)(C)CC2)c1[C@H](OC(C)(C)C)C(=O)OC(C)C]

### Isopropyl (S)-2-(tert-butoxy)-2-(4′-(4, 4-dimethylpiperidin-1-yl)-5-isobutoxy-6′-methyl-6-(trifluoromethyl)-[2, 3′-bipyridin]-5′-yl)acetate

To a 14 mL test tube equipped with a stir bar and (S)-(5-(1-(tert-butoxy)-2-isopropoxy-2-oxoethyl)-4-(4,4-dimethylpiperidin-1-yl)-6-methylpyridin-3-yl)boronic acid (210 mg, 0.500 mmol) was added 6-chloro-3-isobutoxy-2-(trifluoromethyl)pyridine (31.7 mg, 0.125 mmol), potassium phosphate tribasic (954 mg, 4.50 mmol) and SPhos-Pd-G3 (19.46 mg, 0.025 mmol). The flask was sealed with a rubber septum, then was placed under N₂ atm (vac/fill×3). To the flask was added degassed (N₂ bubbling for 5 min) dioxane (1873 μl) and water (624 μl). The test tube was placed in a 60° C. heating block with stirring for t=18 h. The reaction was cooled to RT and diluted with EtOAc and water. The organic layer was washed with brine, collected, dried over MgSO₄, filtered and volatiles evaporated to afford the crude product. The crude product was purified via silica gel chromatography (24 g column, 20-100% EtOAc:Hex) to afford the product isopropyl (S)-2-(tert-butoxy)-2-(4′-(4,4-dimethylpiperidin-1-yl)-5-isobutoxy-6′-methyl-6-(trifluoromethyl)-[2,3′-bipyridin]-5′-yl)acetate (99 mg, 0.167 mmol, 33.4% yield) as a red/brown oil. ESI-MS(+) m/z=594.6 (M+1).

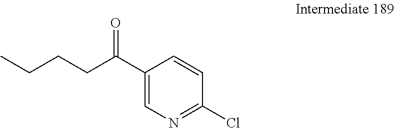

### 1-(6-Chloropyridin-3-yl)pentan-1-one

To a 0° C. solution of 6-chloro-N-methoxy-N-methylnicotinamide (500 mg, 2.492 mmol) in THF (10 mL) was added dropwise butylmagnesium bromide (2.492 mL, 4.98 mmol) and allowed to stir for 15 min at 0° C. before allowing to warm up to RT. The reaction was then stirred for 1 hr. After stirring for 1 hr, the reaction was quenched with sat. aq. ammonium chloride and then extracted with EtOAc (50 mL). The organic layer was washed with brine, collected, dried over MgSO₄, filtered and volatiles evaporated to afford a yellow solid. The crude material was purified on silica gel (24 g column, 5-40% EtOAc:Hex) to afford the product 1-(6-chloropyridin-3-yl)pentan-1-one (425 mg, 2.150 mmol, 86% yield) as a white solid. ¹H NMR (500 MHz, chloroform-d) δ 8.96 (dd, J=2.4, 0.6 Hz, 1H), 8.21 (dd, J=8.4, 2.4 Hz, 1H), 7.46 (dd, J=8.4, 0.6 Hz, 1H), 3.03-2.92 (m, 2H), 1.80-1.68 (m, 2H), 1.49-1.38 (m, 2H), 0.99 (t, J=7.4 Hz, 3H).

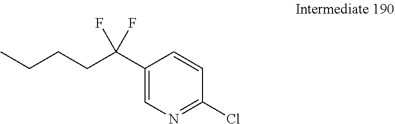

### 2-Chloro-5-(1,1-difluoropentyl)pyridine

Deoxofluor (3 mL, 8.14 mmol) (50% in toluene) was added to a solution of 1-(6-chloropyridin-3-yl)pentan-1-one (425 mg, 2.150 mmol) in toluene (0.5 mL). The reaction was warmed up to 60° C. for 18 hrs. The reaction was cooled to RT and diluted with 1.5 M K₃PO₄ and extracted with EtOAc. The organic layer was washed with brine, collected, dried over MgSO₄, filtered and volatiles evaporated to afford the crude product. The crude product was purified on silica gel (24 g column, 0-20% EtOAc:Hex) to afford the product 2-chloro-5-(1,1-difluoropentyl)pyridine (243 mg, 1.106 mmol, 51.5% yield) as a clear oil. ¹H NMR (500 MHz, chloroform-d) δ 8.56-8.49 (m, 1H), 7.76 (dd, J=8.2, 2.5 Hz, 1H), 7.42 (dd, J=8.4, 0.6 Hz, 1H), 2.22-2.07 (m, 2H), 1.48-1.34 (m, 4H), 0.95-0.90 (m, 3H).

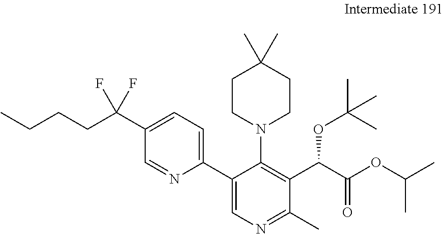 [SMILES: CCCCC(F)(F)c1ccc(-c2cnc(C)c([C@H](OC(C)(C)C)C(=O)OC(C)C)c2N2CCC(C)(C)CC2)nc1]

### Isopropyl (S)-2-(tert-butoxy)-2-(5-(1,1-difluoropentyl)-4′-(4, 4-dimethylpiperidin-1-yl)-6′-methyl-[2, 3′-bipyridin]-5′-yl)acetate

To a 14 mL test tube equipped with a stir bar and (S)-(5-(1-(tert-butoxy)-2-isopropoxy-2-oxoethyl)-4-(4,4-dimethylpiperidin-1-yl)-6-methylpyridin-3-yl)boronic acid (210 mg, 0.500 mmol) was added 2-chloro-5-(1,1-difluoropentyl)pyridine (27.4 mg, 0.125 mmol), potassium phosphate tribasic (954 mg, 4.50 mmol) and SPhos-Pd-G3 (19.46 mg, 0.025 mmol). The flask was sealed with a rubber septum, then was placed under N₂ atm (vac/fill×3). To the flask was added degassed (N₂ bubbling for 5 min) dioxane (1873 μl) and water (624 μl). The test tube was placed in a 60° C. heating block with stirring for t=18 h. The reaction was cooled to RT and diluted with EtOAc and water. The organic layer was washed with brine, collected, dried over MgSO₄, filtered and volatiles evaporated to afford the crude product. The crude product was purified via silica gel chromatography (24 g column, 20-100% EtOAc:Hex) to afford the product isopropyl (S)-2-(tert-butoxy)-2-(5-(1,1-difluoropentyl)-4′-(4,4-dimethylpiperidin-1-yl)-6′-methyl-[2,3′-bipyridin]-5′-yl)acetate (130 mg, 0.232 mmol, 46.5% yield) as a red/brown oil. ESI-MS(+) m/z=560.6 (M+1).

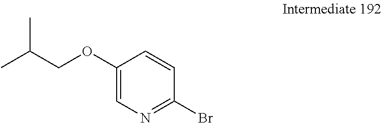

### 2-Bromo-5-isobutoxypyridine

To a 500 mL r.b. flask equipped with a large stir bar was added 6-bromopyridin-3-ol (10.0 g, 57.5 mmol), 2-methylpropan-1-ol (4.26 g, 57.5 mmol), triphenylphosphine (15.8 g, 60.3 mmol) and THF (192 ml). The flask was placed at r.t. To the stirring solution was added DIAD (11.73 ml, 60.3 mmol). At t=15 min the reaction solution was concentrated in vacuo. To the crude reaction product was added hexanes:Et₂O (1:1, 250 mL). The mixture was stirred. A significant amount of crystalline material (OPPh₃) ppt'd and was removed via filtration. The filtrate was concentrated in vacuo and the residue was dissolved in a minimal of acetone and then concentrated onto Celite. The resulting powder was subjected to SiO2 purification on the Biotage (120 g column) 0-100% 10 CVs to afford 2-bromo-5-isobutoxypyridine (12.7 g, 96%) as a clear oil. LCMS Method 4: retention time=1.36 min.; observed ion=230.0, 232.0. ¹H NMR (500 MHz, CHLOROFORM-d) δ 8.07 (d, J=3.0 Hz, 1H), 7.44-7.25 (m, 1H), 7.11 (dd, J=8.8, 3.2 Hz, 1H), 3.76 (d, J=6.5 Hz, 2H), 2.17-2.06 (m, 1H), 1.05 (d, J=6.8 Hz, 6H).

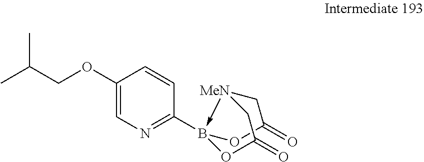

### 2-(5-Isobutoxypyridin-2-yl)-6-methyl-1,3,6, 2-dioxazaborocane-4, 8-dione

To a dry 250 mL r.b. flask equipped with a large stir bar was added 2-bromo-5-isobutoxypyridine (12.7 g, 55.2 mmol). The flask was placed under N₂ atm (vac/fill×3), then to the flask was added THF (97 ml) and triisopropyl borate (12.9 ml, 55.7 mmol). The solution was degassed with N₂ 2×. The flask was cooled in a −78° C. bath. To the solution was added dropwise n-butyllithium in hexanes (22.3 ml, 55.7 mmol) at a rate necessary to avoid build-up of any localized dark discoloration (usually visible in the central vortex with fast stirring). The rate was approximately 0.25-0.50 mL/min. At the completion of the addition the solution slowly began to turn dark yellow. Stirring was maintained for 20 min upon which the solution was observed to be a light amber. The bath was removed and the solution was allowed to warm to r.t. with stirring (t=0). As the solution warmed, the color deepened even more significantly. At t=3 h the reaction solution was carried forward into the distillation step.

The distillation is setup:

A 3-neck 250 mL flask equipped with a large stir bar was charged with methyliminodiacetic acid (16.24 g, 110 mmol) and DMSO (97 ml). The center neck was fitted with a pressure-equalizing addition funnel vented to positive N₂ pressure. Another neck was fitted with a rubber septum through which a thermocouple was inserted to monitor internal temperature. The final neck was fitted with a short-path distillation apparatus collecting in a 100 mL r.b. flask and vented to a bubbler. This provides a slow stream of N₂ carrier gas through the setup and out through the bubbler so that THF vapor does not accumulate.

Distillation:

The reaction solution containing the boronate was transferred to the addition funnel. The 3-neck flask was heated with an oil bath (160° C.). Once the internal temperature had reached 115-120° C. the boronate solution was added dropwise at a rate necessary to maintain an internal temp of 115-120° C. The addition took approximately 20 min. The receiver flask containing the THF was exchanged for an empty 100 mL r.b. flask. The bubbler line connected to the vacuum arm of the distillation apparatus was exchanged for a tube running to the rotovap. The N₂ source was closed. The system was placed under vacuum, slowly ramping upon which the DMSO distilled. The distillation was maintained until only trace DMSO remained. The residue was dissolved in MeCN upon which only the MIDA (white powder) did not dissolve. The mixture was concentrated onto Celite in vacuo. The resulting powder was subjected to SiO₂ purification on the Biotage (0-100% EtOAc/ACN gradient over 10 CVs). TLC was performed with 1:1 EA/ACN looking at it with both UV and then KMnO4 staining (to observe biproduct). The fractions containing the product were collected and concentrated to afford the desired product: 2-(5-isobutoxypyridin-2-yl)-6-methyl-1,3,6,2-dioxazaborocane-4,8-dione (7.3 g, 23.85 mmol, 43.2% yield) as a white solid. LCMS Method 5: retention time=1.35 min.; observed ion=307.2. ¹H NMR (500 MHz, Acetone) δ 8.45-8.38 (m, 1H), 7.60-7.54 (m, 1H), 7.29 (dd, J=8.4, 2.9 Hz, 1H), 4.34 (d, J=16.6 Hz, 2H), 4.17 (d, J=16.6 Hz, 2H), 3.90-3.84 (m, 2H), 2.78 (s, 3H), 2.15-2.09 (m, 1H), 1.05 (d, J=6.8 Hz, 6H).

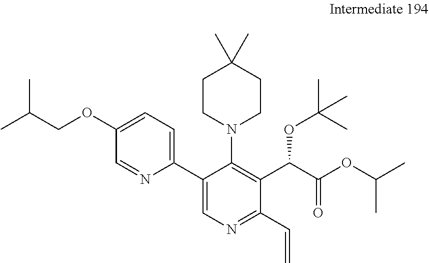 [SMILES: C=Cc1ncc(-c2ccc(OCC(C)C)cn2)c(N2CCC(C)(C)CC2)c1[C@H](OC(C)(C)C)C(=O)OC(C)C]

### Isopropyl (S)-2-(tert-butoxy)-2-(4′-(4, 4-dimethylpiperidin-1-yl)-5-isobutoxy-6′-vinyl-[2, 3′-bipyridin]-5′-yl)acetate

To a dry 10 mL Schlenk flask equipped with a stir bar was added 2-(5-isobutoxypyridin-2-yl)-6-methyl-1,3,6,2-dioxazaborocane-4,8-dione compound with 2-butyl-6-methyl-1,3,6,2-dioxazaborocane-4,8-dione (41.6 mg, 0.080 mmol), isopropyl (S)-2-(5-bromo-4-(4,4-dimethylpiperidin-1-yl)-2-vinylpyridin-3-yl)-2-(tert-butoxy)acetate (25 mg, 0.053 mmol), palladium tetrakis (12.36 mg, 10.70 μmol), diacetoxycopper (4.86 mg, 0.027 mmol) and anhydrous tribasic potassium phosphate, finely ground (56.8 mg, 0.267 mmol). The flask was sealed with a rubber septum, then placed under N₂ atm (vac/fill×3). To the flask was added a degassed (N₂ sparging for 5 min) solution of DMF+diethanolamine (5.62 mg, 0.053 mmol). The flask was placed in a 100° C. oil bath with stirring for t=18 hrs. The reaction mixture was transferred to a 125 mL separatory funnel and was diluted with water:brine (1:1, 50 mL). The mixture was extracted with EtOAc (3×50 mL). The combined organics were washed with water:brine (1:1, 50 mL), then brine (50 mL). The organics were dried over MgSO₄; filtered; then concentrated in vacuo to afford an amber oil. This material was dissolved in a min of acetone, then was concentrated onto Celite in vacuo. The resulting powder was subjected to SiO2 purification to afford the product isopropyl (S)-2-(tert-butoxy)-2-(4′-(4,4-dimethylpiperidin-1-yl)-5-isobutoxy-6′-vinyl-[2,3′-bipyridin]-5′-yl)acetate (18 mg, 0.033 mmol, 62.6% yield) as a dark amber oil. ESI-MS(+) m/z=538.5 (M+1).

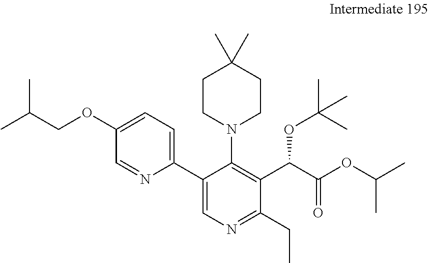 [SMILES: CCc1ncc(-c2ccc(OCC(C)C)cn2)c(N2CCC(C)(C)CC2)c1[C@H](OC(C)(C)C)C(=O)OC(C)C]

### Isopropyl (S)-2-(tert-butoxy)-2-(4′-(4, 4-dimethylpiperidin-1-yl)-6′-ethyl-5-isobutoxy-[2, 3′-bipyridin]-5′-yl)acetate

To an N₂ sparged solution of isopropyl (S)-2-(tert-butoxy)-2-(4′-(4,4-dimethylpiperidin-1-yl)-5-isobutoxy-6′-vinyl-[2,3′-bipyridin]-5′-yl)acetate (18 mg, 0.033 mmol) in MeOH (2 mL) was added Pd—C (3.56 mg, 3.35 μmol) and capped with a rubber septum. H₂ was then bubbled through the solution for 10 minutes. The reaction was left under positive pressure of H₂ for 1 hr. The LCMS indicated the reaction was complete. The reaction was filtered through a 0.45 t nylon frit filter and volatiles evaporated to afford the crude oil. The reaction afforded the expected product isopropyl (S)-2-(tert-butoxy)-2-(4′-(4,4-dimethylpiperidin-1-yl)-6′-ethyl-5-isobutoxy-[2,3′-bipyridin]-5′-yl)acetate (18 mg, 0.033 mmol, 100% yield). ESI-MS(+) m/z=540.6 (M+1).

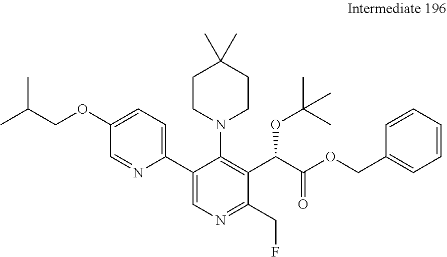 [SMILES: CC(C)COc1ccc(-c2cnc(CF)c([C@H](OC(C)(C)C)C(=O)OCc3ccccc3)c2N2CCC(C)(C)CC2)nc1]

### Benzyl (S)-2-(tert-butoxy)-2-(4′-(4, 4-dimethylpiperidin-1-yl)-6′-(fluoromethyl)-5-isobutoxy-[2, 3′-bipyridin]-5′-yl)acetate

To a dry 10 mL Schlenk flask equipped with a stir bar was added 2-(5-isobutoxypyridin-2-yl)-6-methyl-1,3,6,2-dioxazaborocane-4,8-dione compound with 2-butyl-6-methyl-1,3,6,2-dioxazaborocane-4,8-dione (87 mg, 0.168 mmol), isopropyl (S)-2-(5-bromo-4-(4,4-dimethylpiperidin-1-yl)-2-(fluoromethyl)pyridin-3-yl)-2-(tert-butoxy)acetate (53 mg, 0.112 mmol), palladium tetrakis (25.9 mg, 0.022 mmol), diacetoxycopper (10.17 mg, 0.056 mmol) and anhydrous tribasic potassium phosphate, finely ground (119 mg, 0.560 mmol). The flask was sealed with a rubber septum, then placed under N₂ atm (vac/fill×3). To the flask was added a degassed (N₂ sparging for 5 min) solution of DMF+diethanolamine (11.77 mg, 0.112 mmol). The flask was placed in a 100° C. oil bath with stirring for t=18 h. The reaction mixture was transferred to a 125 mL separatory funnel and was diluted with water:brine (1:1, 50 mL). The mixture was extracted with EtOAc (3×50 mL). The combined organics were washed with water:brine (1:1, 50 mL), then brine (50 mL). The organics were dried over MgSO₄; filtered; then concentrated in vacuo to afford an amber oil. This material was dissolved in a min of acetone, then was concentrated onto Celite in vacuo. The resulting powder was subjected to SiO2 purification to afford the product benzyl (S)-2-(tert-butoxy)-2-(4′-(4,4-dimethylpiperidin-1-yl)-6′-(fluoromethyl)-5-isobutoxy-[2,3′-bipyridin]-5′-yl)acetate (23 mg, 0.039 mmol, 34.7% yield) as an amber oil. ESI-MS(+) m/z=592.4 (M+1).

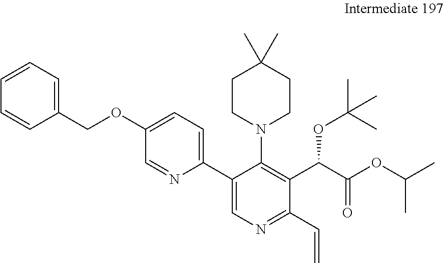 [SMILES: C=Cc1ncc(-c2ccc(OCc3ccccc3)cn2)c(N2CCC(C)(C)CC2)c1[C@H](OC(C)(C)C)C(=O)OC(C)C]

### Isopropyl (S)-2-(5-(benzyloxy)-4′-(4, 4-dimethylpiperidin-1-yl)-6′-vinyl-[2, 3′-bipyridin]-5′-yl)-2-(tert-butoxy)acetate

To a dry 10 mL Schlenk flask equipped with a stir bar was added 2-(5-(benzyloxy)pyridin-2-yl)-6-methyl-1,3,6,2-dioxazaborocane-4,8-dione (1.091 g, 3.21 mmol), isopropyl (S)-2-(5-bromo-4-(4,4-dimethylpiperidin-1-yl)-2-vinylpyridin-3-yl)-2-(tert-butoxy)acetate (1 g, 2.139 mmol), palladium tetrakis (0.494 g, 0.428 mmol), diacetoxycopper (0.194 g, 1.070 mmol) and anhydrous tribasic potassium phosphate, finely ground (2.270 g, 10.70 mmol). The flask was sealed with a rubber septum, then placed under N₂ atm (vac/fill×3). To the flask was added a degassed (N₂ sparging for 5 min) solution of DMF+diethanolamine (0.225 g, 2.139 mmol). The flask was placed in a 100° C. oil bath with stirring for t=18 h. The reaction mixture was transferred to a 125 mL separatory funnel and was diluted with water:brine (1:1, 50 mL). The mixture was extracted with EtOAc (3×50 mL). The combined organics were washed with water:brine (1:1, 50 mL), then brine (50 mL). The organics were dried over MgSO₄; filtered; then concentrated in vacuo to afford an amber oil. This material was dissolved in a min of acetone, then was concentrated onto Celite in vacuo. The resulting powder was subjected to SiO2 purification to afford the product isopropyl (S)-2-(5-(benzyloxy)-4′-(4,4-dimethylpiperidin-1-yl)-6′-vinyl-[2,3′-bipyridin]-5′-yl)-2-(tert-butoxy)acetate (670 mg, 1.172 mmol, 54.8% yield). ¹H NMR (400 MHz, chloroform-d) δ 8.52 (d, J=2.7 Hz, 1H), 8.37 (s, 1H), 7.53-7.48 (m, 2H), 7.45 (t, J=7.5 Hz, 2H), 7.42-7.32 (m, 4H), 6.33 (dd, J=16.8, 2.1 Hz, 1H), 6.10 (br s, 1H), 5.43 (dd, J=10.6, 2.1 Hz, 1H), 5.21 (s, 2H), 5.10 (dt, J=12.6, 6.2 Hz, 1H), 3.44-3.22 (m, 2H), 1.23 (d, J=6.1 Hz, 3H), 1.19 (s, 9H), 1.17 (d, J=6.4 Hz, 3H), 0.98-0.80 (m, 6H). 6 protons of the methylenes on the piperidine were not observed in the HNMR spectrum. ESI-MS(+) m/z=572.6 (M+1).

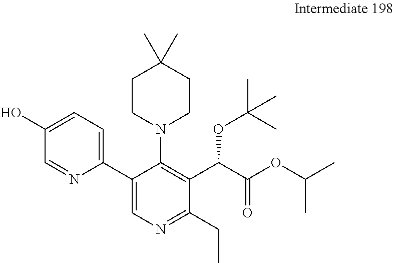 [SMILES: CCc1ncc(-c2ccc(O)cn2)c(N2CCC(C)(C)CC2)c1[C@H](OC(C)(C)C)C(=O)OC(C)C]

### (S)-2-(tert-Butoxy)-2-(4′-(4, 4-dimethylpiperidin-1-yl)-6′-ethyl-5-hydroxy-[2, 3′-bipyridin]-5′-yl)acetate

To an N₂ sparged solution of isopropyl (S)-2-(5-(benzyloxy)-4′-(4,4-dimethylpiperidin-1-yl)-6′-vinyl-[2,3′-bipyridin]-5′-yl)-2-(tert-butoxy)acetate (670 mg, 1.172 mmol) in MeOH (15 mL) was added Pd—C (125 mg, 0.117 mmol) and capped with a rubber septum. H₂ was then bubbled through the solution for 10 minutes. The reaction was left under positive pressure of H₂ for 1 hr. The LCMS indicated the reaction was complete. The reaction was filtered through a 0.45 g nylon frit filter and volatiles evaporated to afford the product isopropyl (S)-2-(tert-butoxy)-2-(4′-(4,4-dimethylpiperidin-1-yl)-6′-ethyl-5-hydroxy-[2,3′-bipyridin]-5′-yl)acetate (447 mg, 0.924 mmol, 79% yield). ¹H NMR (400 MHz, chloroform-d) δ 8.39 (d, J=2.4 Hz, 1H), 8.26 (s, 1H), 7.31 (br d, J=2.7 Hz, 1H), 7.24-7.19 (m, 1H), 6.05 (br s, 1H), 5.17-5.08 (m, 1H), 3.05 (dq, J=13.9, 7.1 Hz, 1H), 2.97-2.88 (m, 1H), 1.38-1.35 (m, 3H), 1.25 (d, J=6.4 Hz, 3H), 1.23-1.19 (m, 12H), 0.89 (br s, 6H). 8 protons on the methylenes on the piperidine ring were not observed in the HNMR spectrum. ESI-MS(+) m/z=484.5 (M+1).

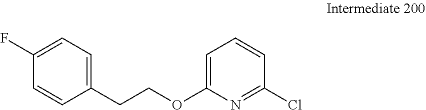 [SMILES: Fc1ccc(CCOc2cccc(Cl)n2)cc1]

### 2-Chloro-6-(4-fluorophenethoxy)pyridine

To a 50 mL round bottom flask equipped with a stir bar was added 6-chloropyridin-2-ol (250 mg, 1.930 mmol), 2-(4-fluorophenyl)ethan-1-ol (0.242 mL, 1.930 mmol), triphenylphosphine (607 mg, 2.316 mmol) and THF (10 mL). To the stirred solution was added DIAD (0.450 mL, 2.316 mmol). The solution warmed to a mild reflux, then cooled within 5 minutes. The solution was stirred at r.t. for 2 hrs. The reaction solution was concentrated in vacuo and the resulting oil was diluted with a min of acetone, then concentrated onto Celite in vacuo. The resulting powder was subjected to silica gel chromatography (40 g column, 5-40% EtOAc:Hex) to afford the product 2-chloro-6-(4-fluorophenethoxy)pyridine (443 mg, 1.760 mmol, 91% yield) as a white solid. ¹H NMR (500 MHz, chloroform-d) δ 7.52 (dd, J=8.2, 7.6 Hz, 1H), 7.26 (dd, J=8.7, 5.4 Hz, 2H), 7.04-6.99 (m, 2H), 6.91 (dd, J=7.6, 0.6 Hz, 1H), 6.65 (dd, J=8.2, 0.6 Hz, 1H), 4.51 (t, J=6.9 Hz, 2H), 3.07 (t, J=6.9 Hz, 2H). ESI-MS(+) m/z=252.1 (M+1).

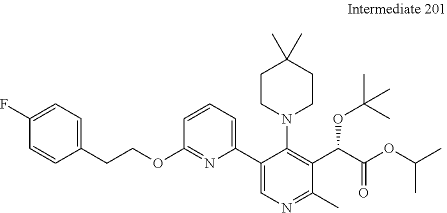 [SMILES: Cc1ncc(-c2cccc(OCCc3ccc(F)cc3)n2)c(N2CCC(C)(C)CC2)c1[C@H](OC(C)(C)C)C(=O)OC(C)C]

### Isopropyl (S)-2-(tert-butoxy)-2-(4′-(4, 4-dimethylpiperidin-1-yl)-6-(4-fluorophenethoxy)-6′-methyl-[2, 3′-bipyridin]-5′-yl)acetate

To a 14 mL test tube equipped with a stir bar and (S)-(5-(1-(tert-butoxy)-2-isopropoxy-2-oxoethyl)-4-(4,4-dimethylpiperidin-1-yl)-6-methylpyridin-3-yl)boronic acid (210 mg, 0.500 mmol) was added 2-chloro-6-(4-fluorophenethoxy)pyridine (31.4 mg, 0.125 mmol), potassium phosphate tribasic (954 mg, 4.50 mmol) and SPhos-Pd-G3 (19.46 mg, 0.025 mmol). The flask was sealed with a rubber septum, then was placed under N₂ atm (vac/fill×3). To the flask was added degassed (N₂ bubbling for 5 min) dioxane (1873 μl) and water (624 μl). The test tube was placed in a 60° C. heating block with stirring for t=18 h. The reaction was cooled to RT and diluted with EtOAc and water. The organic layer was washed with brine, collected, dried over MgSO₄, filtered and volatiles evaporated to afford the crude product. The crude product was purified via silica gel chromatography (24 g column, 20-100% EtOAc:Hex) to afford the product isopropyl (S)-2-(tert-butoxy)-2-(4′-(4,4-dimethylpiperidin-1-yl)-6-(4-fluorophenethoxy)-6′-methyl-[2,3′-bipyridin]-5′-yl)acetate (132 mg, 0.223 mmol, 44.6% yield) as a red/brown oil. ESI-MS(+) m/z=592.4 (M+1).

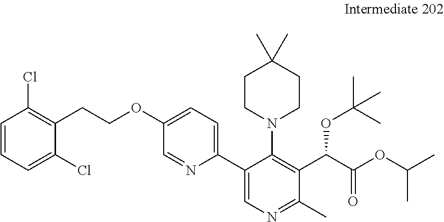 [SMILES: Cc1ncc(-c2ccc(OCCc3c(Cl)cccc3Cl)cn2)c(N2CCC(C)(C)CC2)c1[C@H](OC(C)(C)C)C(=O)OC(C)C]

### (S)-Isopropyl 2-(tert-butoxy)-2-(5-(2, 6-dichlorophenethoxy)-4′-(4, 4-dimethylpiperidin-1-yl)-6′-methyl-[2, 3′-bipyridin]-5′-yl)acetate

A mixture of (S)-isopropyl 2-(tert-butoxy)-2-(4′-(4,4-dimethylpiperidin-1-yl)-5-hydroxy-6′-methyl-[2,3′-bipyridin]-5′-yl)acetate (300 mg, 0.639 mmol) and sodium carbonate (508 mg, 4.79 mmol) in dichloromethane (15 mL), 2,6-dichlorophenethyl methanesulfonate (860 mg, 3.19 mmol) was added and stirred for 16 hour at 25° C. Then, the reaction mixture was concentrated under vacuo and the reissue was treated with aq. HCl (1N, 0.5 ml) and purified by Prep-HPLC to give desired product (S)-isopropyl 2-(tert-butoxy)-2-(5-(2,6-dichlorophenethoxy)-4′-(4,4-dimethylpiperidin-1-yl)-6′-methyl-[2,3′-bipyridin]-5′-yl)acetate (300 mg, 0.387 mmol, 60.7% yield) as white solid. LCMS [M+H]=642.2.

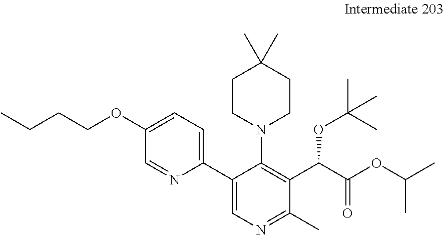 [SMILES: CCCCOc1ccc(-c2cnc(C)c([C@H](OC(C)(C)C)C(=O)OC(C)C)c2N2CCC(C)(C)CC2)nc1]

### (S)-Isopropyl 2-(tert-butoxy)-2-(5-butoxy-4′-(4, 4-dimethylpiperidin-1-yl)-6′-methyl-[2, 3′-bipyridin]-5′-yl)acetate

A mixture of (S)-isopropyl 2-(tert-butoxy)-2-(4′-(4,4-dimethylpiperidin-1-yl)-5-hydroxy-6′-methyl-[2,3′-bipyridin]-5′-yl)acetate (250 mg, 0.532 mmol), 1-bromopropane (524 mg, 4.26 mmol) and K₂CO₃ (441 mg, 3.19 mmol) in DMF (4 mL) stirred for 16 h at rt. The suspension was filtered and filtrate was purified by preparative-HPLC to give the desired product (S)-isopropyl 2-(tert-butoxy)-2-(5-butoxy-4′-(4,4-dimethylpiperidin-1-yl)-6′-methyl-[2,3′-bipyridin]-5′-yl)acetate (240 mg, 0.457 mmol, 86% yield) as white solid. LCMS [M+H]=526.3.

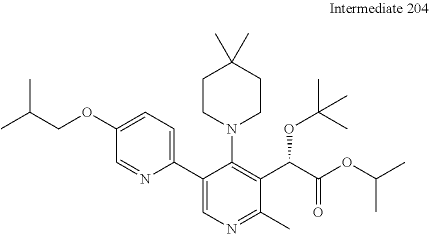 [SMILES: Cc1ncc(-c2ccc(OCC(C)C)cn2)c(N2CCC(C)(C)CC2)c1[C@H](OC(C)(C)C)C(=O)OC(C)C]

### (S)-Isopropyl 2-(tert-butoxy)-2-(4′-(4, 4-dimethylpiperidin-1-yl)-5-isobutoxy-6′-methyl-[2, 3′-bipyridin]-5′-yl)acetate

A mixture of (S)-isopropyl 2-(tert-butoxy)-2-(4′-(4,4-dimethylpiperidin-1-yl)-5-hydroxy-6′-methyl-[2,3′-bipyridin]-5′-yl)acetate (500 mg, 1.065 mmol) in acetonitrile (20 mL), K₂CO₃ (736 mg, 5.32 mmol) and 1-bromo-2-methylpropane (438 mg, 3.19 mmol) was stirred for 20 hours at 70° C. The reaction mixture was filtered, concentrated and purified by silica gel chromatography eluting with EtOAc/Pet. ether (from 10:1 to 1:1) to afford (S)-isopropyl 2-(tert-butoxy)-2-(4′-(4,4-dimethylpiperidin-1-yl)-5-isobutoxy-6′-methyl-[2,3′-bipyridin]-5′-yl)acetate (500 mg, 0.951 mmol, 89% yield) as white yellow oil. LCMS [M+H]=526.3.

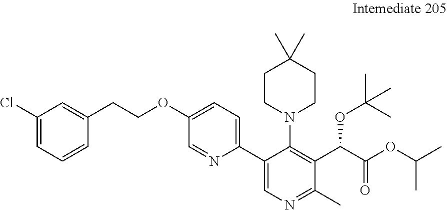 [SMILES: Cc1ncc(-c2ccc(OCCc3cccc(Cl)c3)cn2)c(N2CCC(C)(C)CC2)c1[C@H](OC(C)(C)C)C(=O)OC(C)C]

### (S)-Isopropyl 2-(tert-butoxy)-2-(5-(3-chlorophenethoxy)-4′-(4, 4-dimethylpiperidin-1-yl)-6′-methyl-[2, 3′-bipyridin]-5′-yl)acetate

To a stirred mixture of 3-chlorophenethyl methanesulfonate (750 mg, 3.19 mmol), (S)-isopropyl 2-(tert-butoxy)-2-(4′-(4,4-dimethylpiperidin-1-yl)-5-hydroxy-6′-methyl-[2,3′-bipyridin]-5′-yl)acetate (300 mg, 0.639 mmol) in DMF (15 mL) was added K₂CO₃ (662 mg, 4.79 mmol) and heated at 60° C. for 2 hours. Then, reaction mixture was diluted by DCM 20 ml and water (5 ml), organic layer separated and aqueous layer extracted by DCM (20 ml). The combined organic layers were dried over Na₂SO₄ and concentrated. The residue was purified by silica gel column (Pet. ether/EtOAc=10:1) to afford desired product (S)-isopropyl 2-(tert-butoxy)-2-(5-(3-chlorophenethoxy)-4′-(4,4-dimethylpiperidin-1-yl)-6′-methyl-[2,3′-bipyridin]-5′-yl)acetate (300 mg, 0.486 mmol, 76% yield) as a white solid. LCMS [M+H]=608.3.

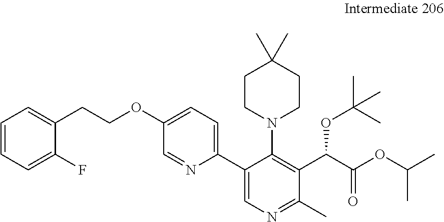 [SMILES: Cc1ncc(-c2ccc(OCCc3ccccc3F)cn2)c(N2CCC(C)(C)CC2)c1[C@H](OC(C)(C)C)C(=O)OC(C)C]

### (S)-Isopropyl 2-(tert-butoxy)-2-(4′-(4, 4-dimethylpiperidin-1-yl)-5-(2-fluorophenethoxy)-6′-methyl-[2, 3′-bipyridin]-5′-yl)acetate

To a solution of (S)-isopropyl 2-(tert-butoxy)-2-(4′-(4,4-dimethylpiperidin-1-yl)-5-hydroxy-6′-methyl-[2,3′-bipyridin]-5′-yl)acetate (300 mg, 0.639 mmol) in acetonitrile (15 mL) was added cesium carbonate (624 mg, 1.916 mmol) and the mixture was stirred overnight at 75° C. The reaction mixture was filtered and the filtrate was concentrated. The residue was purified by silica gel column (Pet. ether/EtOAc, 8:1) to afford the desired product (S)-isopropyl 2-(tert-butoxy)-2-(4′-(4,4-dimethylpiperidin-1-yl)-5-(2-fluorophenethoxy)-6′-methyl-[2,3′-bipyridin]-5′-yl)acetate (210 mg, 0.341 mmol, 53.3% yield) as yellow oil. LCMS [M+H]=592.3.

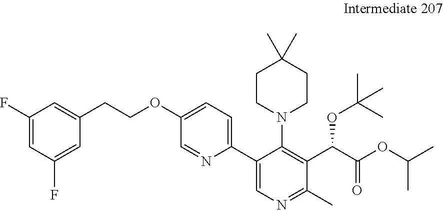 [SMILES: Cc1ncc(-c2ccc(OCCc3cc(F)cc(F)c3)cn2)c(N2CCC(C)(C)CC2)c1[C@H](OC(C)(C)C)C(=O)OC(C)C]

### (S)-Isopropyl 2-(tert-butoxy)-2-(5-(3, 5-difluorophenethoxy)-4′-(4, 4-dimethylpiperidin-1-yl)-6′-methyl-[2, 3′-bipyridin]-5′-yl)acetate

To a solution of (S)-isopropyl 2-(tert-butoxy)-2-(4′-(4,4-dimethylpiperidin-1-yl)-5-hydroxy-6′-methyl-[2,3′-bipyridin]-5′-yl)acetate (200 mg, 0.426 mmol) in acetonitrile (20 mL) was added potassium carbonate (294 mg, 2.129 mmol) and stirred overnight at 75° C. The mixture was filtered and the filtrate was concentrated. The residue was purified by silica gel column (Pet. ether/EtOAc 1:1) to afford the desired product (S)-isopropyl 2-(tert-butoxy)-2-(5-(3,5-difluorophenethoxy)-4′-(4,4-dimethylpiperidin-1-yl)-6′-methyl-[2,3′-bipyridin]-5′-yl)acetate (210 mg, 0.313 mmol, 73.6% yield) as yellow oil. LCMS [M+H]=610.2.

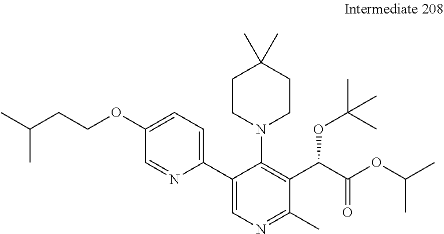 [SMILES: Cc1ncc(-c2ccc(OCCC(C)C)cn2)c(N2CCC(C)(C)CC2)c1[C@H](OC(C)(C)C)C(=O)OC(C)C]

### (S)-Isopropyl 2-(tert-butoxy)-2-(4′-(4, 4-dimethylpiperidin-1-yl)-5-(isopentyloxy)-6′-methyl-[2, 3′-bipyridin]-5′-yl)acetate

A mixture of (S)-isopropyl 2-(tert-butoxy)-2-(4′-(4,4-dimethylpiperidin-1-yl)-5-hydroxy-6′-methyl-[2,3′-bipyridin]-5′-yl)acetate (240 mg, 0.511 mmol), 1-bromo-3-methylbutane (116 mg, 0.767 mmol) and K₂CO₃ (212 mg, 1.533 mmol) in DMF (8 mL) was heated at 80° C. overnight. Then, diluted with EtOAc (20 ml) and water (15 ml), aqueous phase separated and the organic phase was washed with water (10 ml×2), dried with Na₂SO₄ and concentrated. The residue was purified by Prep-TLC (EtOAc:Pet. ether; 1:2) to afford (S)-isopropyl 2-(tert-butoxy)-2-(4′-(4,4-dimethylpiperidin-1-yl)-5-(isopentyloxy)-6′-methyl-[2,3′-bipyridin]-5′-yl)acetate (240 mg, 0.445 mmol, 87% yield). LCMS [M+H]=540.

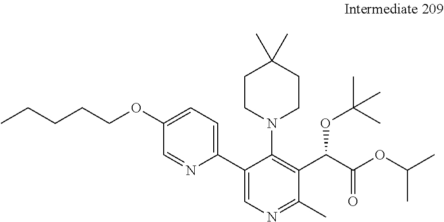 [SMILES: CCCCCOc1ccc(-c2cnc(C)c([C@H](OC(C)(C)C)C(=O)OC(C)C)c2N2CCC(C)(C)CC2)nc1]

### (S)-Isopropyl 2-(tert-butoxy)-2-(4′-(4, 4-dimethylpiperidin-1-yl)-6′-methyl-5-(pentyloxy)-[2, 3′-bipyridin]-5′-yl)acetate

A mixture of (S)-isopropyl 2-(tert-butoxy)-2-(4′-(4,4-dimethylpiperidin-1-yl)-5-hydroxy-6′-methyl-[2,3′-bipyridin]-5′-yl)acetate (200 mg, 0.426 mmol), 1-bromopentane (96 mg, 0.639 mmol) and K₂CO₃ (294 mg, 2.129 mmol) in DMF (8 mL) was heated at 80° C. overnight. Then, the reaction mixture was diluted with EtOAc (20 ml) and water (15 ml). The aqueous phase separated and organic phase was washed with water (10 ml×2), dried with Na₂SO₄ and concentrated. The residue was purified by Prep-TLC (EtOAc:Pet. ether; 1:2) to afford (S)-isopropyl 2-(tert-butoxy)-2-(4′-(4,4-dimethylpiperidin-1-yl)-6′-methyl-5-(pentyloxy)-[2,3′-bipyridin]-5′-yl)acetate (220 mg, 0.408 mmol, 96% yield). LCMS [M+H]=540.

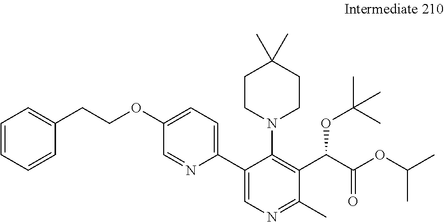 [SMILES: Cc1ncc(-c2ccc(OCCc3ccccc3)cn2)c(N2CCC(C)(C)CC2)c1[C@H](OC(C)(C)C)C(=O)OC(C)C]

### (S)-Isopropyl 2-(tert-butoxy)-2-(4′-(4, 4-dimethylpiperidin-1-yl)-6′-methyl-5-phenethoxy-[2, 3′-bipyridin]-5′-yl)acetate

To a mixture of (S)-isopropyl 2-(tert-butoxy)-2-(4′-(4,4-dimethylpiperidin-1-yl)-5-hydroxy-6′-methyl-[2,3′-bipyridin]-5′-yl)acetate (200 mg, 0.426 mmol), (2-bromoethyl)benzene (394 mg, 2.129 mmol) in DMF (2 mL) was added K₂CO₃ (589 mg, 4.26 mmol) and stirred at rt for 16 hr. Then, diluted with water (20 ml) and EtOAc (10 ml), organic phase separated, washed with water (20 ml×2), brine (20 ml), dried with Na₂SO₄ and concentrated. The residue was purified by silica gel column (Pet. ether/EtOAc; 2:1) to afford the desired product (S)-isopropyl 2-(tert-butoxy)-2-(4′-(4,4-dimethylpiperidin-1-yl)-6′-methyl-5-phenethoxy-[2,3′-bipyridin]-5′-yl)acetate (200 mg, 0.349 mmol, 82% yield) as a white solid. LCMS [M+H]=574.0.

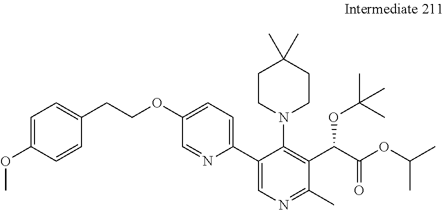 [SMILES: COc1ccc(CCOc2ccc(-c3cnc(C)c([C@H](OC(C)(C)C)C(=O)OC(C)C)c3N3CCC(C)(C)CC3)nc2)cc1]

### (S)-Isopropyl 2-(tert-butoxy)-2-(4′-(4, 4-dimethylpiperidin-1-yl)-5-(4-methoxyphenethoxy)-6′-methyl-[2, 3′-bipyridin]-5′-yl)acetate

To a solution of (S)-isopropyl 2-(tert-butoxy)-2-(4′-(4,4-dimethylpiperidin-1-yl)-5-hydroxy-6′-methyl-[2,3′-bipyridin]-5′-yl)acetate (200 mg, 0.426 mmol) in DMF (5 mL) was added 1-(2-bromoethyl)-4-methoxybenzene (733 mg, 3.41 mmol), K2CO3 (353 mg, 2.56 mmol). The resulting mixture was stirred at 25° C. for about 8 hours and diluted with Et₂O (100 ml). The organic layer was washed with water (40 ml), dried over Na₂SO₄, filtered and concentrated to give crude product. The crude product was purified by prep-HPLC to give the desired product (S)-isopropyl 2-(tert-butoxy)-2-(4′-(4,4-dimethylpiperidin-1-yl)-5-(4-methoxyphenethoxy)-6′-methyl-[2,3′-bipyridin]-5′-yl)acetate (250 mg, 0.414 mmol, 97% yield) as a yellow oil. LCMS (M+H)=604.3.

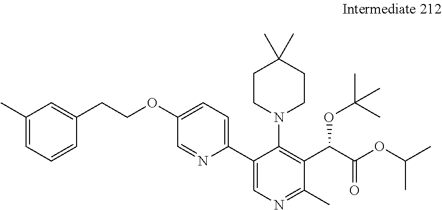 [SMILES: Cc1cccc(CCOc2ccc(-c3cnc(C)c([C@H](OC(C)(C)C)C(=O)OC(C)C)c3N3CCC(C)(C)CC3)nc2)c1]

### (S)-Isopropyl 2-(tert-butoxy)-2-(4′-(4, 4-dimethylpiperidin-1-yl)-6′-methyl-5-(3-methylphenethoxy)-[2, 3′-bipyridin]-5′-yl)acetate

A mixture of (S)-isopropyl 2-(tert-butoxy)-2-(4′-(4,4-dimethylpiperidin-1-yl)-5-hydroxy-6′-methyl-[2,3′-bipyridin]-5′-yl)acetate (300 mg, 0.639 mmol), 1-(2-bromoethyl)-3-methylbenzene (191 mg, 0.958 mmol) and K2CO3 (441 mg, 3.19 mmol) in acetonitrile (8 mL) was heated at 80° C. for 1 h. Then, 1-(2-bromoethyl)-3-methylbenzene (191 mg, 0.958 mmol) was added and the mixture was stirred for 2 h at 80° C. The reaction mixture was diluted with EtOAc (20 ml) and water (15 ml), organic phase separated and washed with water (10 ml×2), dried over Na₂SO₄, concentrated. The residue was purified by Prep-TLC (EtOAc:Pet. ether; 1:2) to afford (S)-isopropyl 2-(tert-butoxy)-2-(4′-(4,4-dimethylpiperidin-1-yl)-6′-methyl-5-(3-methylphenethoxy)-[2,3′-bipyridin]-5′-yl)acetate (280 mg, 0.476 mmol, 74.6% yield). LCMS [M+H]=589.

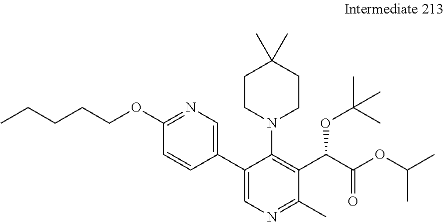 [SMILES: CCCCCOc1ccc(-c2cnc(C)c([C@H](OC(C)(C)C)C(=O)OC(C)C)c2N2CCC(C)(C)CC2)cn1]

### (S)-Isopropyl 2-(tert-butoxy)-2-(4-(4, 4-dimethylpiperidin-1-yl)-6-methyl-6′-(pentyloxy)-[3, 3′-bipyridin]-5-yl)acetate

A mixture of pentan-1-ol (65 mg, 0.737 mmol) and sodium hydride (30 mg, 1.250 mmol) in THF (10 mL) was stirred 30 mins. To the reaction mixture was added (S)-isopropyl 2-(tert-butoxy)-2-(4-(4,4-dimethylpiperidin-1-yl)-6′-fluoro-6-methyl-[3,3′-bipyridin]-5-yl)acetate (70 mg, 0.148 mmol) and stirred at 25° C. After 4 h, the reaction mixture was diluted with H₂O (100 mL) and extracted with EtOAc (150 mL×3). The organic phase was dried over Na₂SO₄ and concentrated. The residue was purified on silica gel column (Pet. ether/EtOAc 4:1) to afford the desired product (S)-isopropyl 2-(tert-butoxy)-2-(4-(4,4-dimethylpiperidin-1-yl)-6-methyl-6′-(pentyloxy)-[3,3′-bipyridin]-5-yl)acetate (60 mg, 65.2% yield). LCMS [M+H]=540.3.

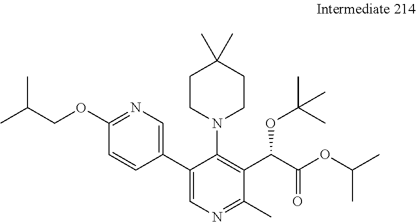 [SMILES: Cc1ncc(-c2ccc(OCC(C)C)nc2)c(N2CCC(C)(C)CC2)c1[C@H](OC(C)(C)C)C(=O)OC(C)C]

### (S)-Isopropyl 2-(tert-butoxy)-2-(4-(4, 4-dimethylpiperidin-1-yl)-6′-isobutoxy-6-methyl-[3,3′-bipyridin]-5-yl)acetate

A mixture of 2-methylpropan-1-ol (11.00 mg, 0.148 mmol) and sodium hydride (3.56 mg, 0.148 mmol) in THF (15 mL) was stirred 30 mins. Then, (S)-isopropyl 2-(tert-butoxy)-2-(4-(4,4-dimethylpiperidin-1-yl)-6′-fluoro-6-methyl-[3,3′-bipyridin]-5-yl)acetate (70 mg, 0.148 mmol) was added and stirred at 25° C. After 4 h, the reaction mixture was diluted with H₂O (100 mL), extracted with EtOAc (150 mL×3) and dried over Na₂SO₄ and concentrated. The residue was purified on silica gel column (Pet. ether/EtOAc 4:1) to afford the desired product (S)-isopropyl 2-(tert-butoxy)-2-(4-(4,4-dimethylpiperidin-1-yl)-6′-isobutoxy-6-methyl-[3,3′-bipyridin]-5-yl)acetate (75 mg, 79% yield) as a white solid. LCMS [M+H]=526.2.

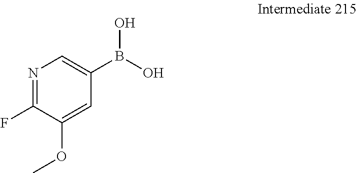

### (6-Fluoro-5-methoxypyridin-3-yl)boronic acid

To a mixture of 4,4,4′,4′,5,5,5′,5′-octamethyl-2,2′-bi(1,3,2-dioxaborolane) (2.465 g, 9.71 mmol), potassium acetate (1.429 g, 14.56 mmol) and 5-bromo-2-fluoro-3-methoxypyridine (1 g, 4.85 mmol) in 1,4-dioxane (15 mL) was added 1,1′-bis(diphenylphosphino)ferrocenedichloro palladium(II) dichloromethane complex (0.355 g, 0.485 mmol). The mixture was stirred overnight at 83° C. under the nitrogen. Then, the reaction mixture was taken up in. EtOAc (300 ml) and H₂O (150 mL). Organic layer was separated and washed with H₂O (100 mL×3), dried with Na₂SO₄ and concentrated. The residue was purified by silica gel column (Pet. ether/EtOAc 3:1) to afford the desired product (6-fluoro-5-methoxypyridin-3-yl)boronic acid (0.67 g, 76% yield). LCMS [M+H]=172.1.

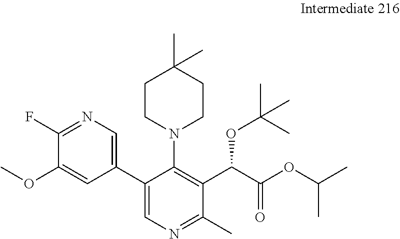 [SMILES: COc1cc(-c2cnc(C)c([C@H](OC(C)(C)C)C(=O)OC(C)C)c2N2CCC(C)(C)CC2)cnc1F]

### (S)-Isopropyl 2-(tert-butoxy)-2-(4-(4, 4-dimethylpiperidin-1-yl)-6′-fluoro-5′-methoxy-6-methyl-[3, 3′-bipyridin]-5-yl)acetate

To a mixture of (S)-isopropyl 2-(5-bromo-4-(4,4-dimethylpiperidin-1-yl)-2-methylpyridin-3-yl)-2-(tert-butoxy)acetate (500 mg, 1.098 mmol), sodium carbonate (233 mg, 2.196 mmol), (6-fluoro-5-methoxypyridin-3-yl)boronic acid (281 mg, 1.647 mmol) in 1,4-dioxane (30 mL) and water (5.0 mL) was added (Ph₃P)₄Pd (254 mg, 0.220 mmol). The flask was placed under N₂ atm (vac/fill×3) and stirred at 84° C. overnight. Then, taken up in EtOAc (400 mL) and H₂O (150 mL), organic layer was separated, washed with H₂O (200 mL×3), dried with Na₂SO₄ and concentrated. The residue was purified by silica gel column (Pet. ether/EtOAC 3:1) to afford the desired product (S)-isopropyl 2-(tert-butoxy)-2-(4-(4,4-dimethylpiperidin-1-yl)-6′-fluoro-5′-methoxy-6-methyl-[3,3′-bipyridin]-5-yl)acetate (100 mg, 16.52% yield). LCMS [M+H]=502.2.

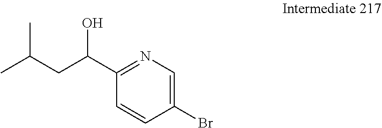

### 1-(5-Bromopyridin-2-yl)-3-methylbutan-1-ol

To a pre-cooled solution of 5-bromopicolinaldehyde (8 g, 43.0 mmol) in anhydrous THF (100 ml) was added dropwise the isobutylmagnesium bromide (86 mL, 86 mmol). The mixture was stirred at rt. for 3 hours and carefully quenched with ice-water. The solvent was removed and water (100 ml) was added to residue and extracted with EtOAc (100 mL×3). The combined organic phases were dried over NaSO4 and concentrated. The crude was purified by column chromatography to give 1-(5-bromopyridin-2-yl)-3-methylbutan-1-ol (4.3 g, 36%). LCMS [M+H]=244.1.

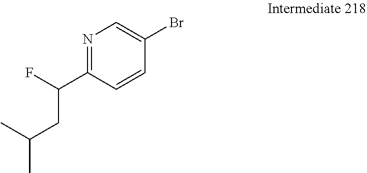

### 5-Bromo-2-(1-fluoro-3-methylbutyl) pyridine

To a solution of 1-(5-bromopyridin-2-yl)-3-methylbutan-1-ol (800 mg, 3.28 mmol) in dichloromethane (15 mL) was added diethylaminosulfur trifluoride (2113 mg, 13.11 mmol) below 0° C. The mixture was stirred overnight at 35° C. under nitrogen. Then, solvent was removed, and EtOAc (200 mL) and saturate solution of NaHCO₃ (100 mL) were added. The organic phase was separated and washed with saturate solution of NaHCO₃ (100 mL×3), dried over Na₂SO₄ and concentrated. The residue was purified by Prep-HPLC to afford the desired product 5-bromo-2-(1-fluoro-3-methylbutyl) pyridine (320 mg, 39.7% yield). LCMS [M+H]=242.0.

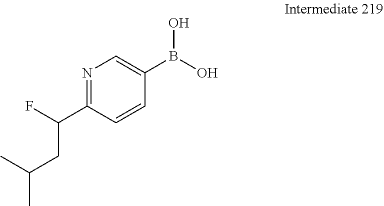

### (6-(1-Fluoro-3-methylbutyl)pyridin-3-yl)boronic acid

To a solution of 5-bromo-2-(1-fluoro-3-methylbutyl)pyridine (220 mg, 0.894 mmol), 1,1′-bis(diphenylphosphino)ferro-cenedichloro palladium(II) dichloromethane complex (65.4 mg, 0.089 mmol) in 1,4-dioxane (15 mL) was added potassium acetate (263 mg, 2.68 mmol) and stirred overnight at 83° C. under nitrogen. Then, the reaction mixture was diluted with EtOAc (300 mL) and H₂O (150 mL), organic layer was separated, washed with H₂O (100 mL×3), dried over Na₂SO₄ and concentrated. The residue was purified on silica gel column (Pet. ether/EtOAc 20:1) to afford the desired product (6-(1-fluoro-3-methylbutyl)pyridin-3-yl)boronic acid (300 mg, 135% yield). LCMS [M+H]=212.2.

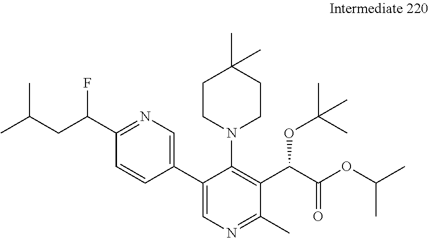 [SMILES: Cc1ncc(-c2ccc(C(F)CC(C)C)nc2)c(N2CCC(C)(C)CC2)c1[C@H](OC(C)(C)C)C(=O)OC(C)C]

### (2S)-Isopropyl 2-(tert-butoxy)-2-(4-(4, 4-dimethylpiperidin-1-yl)-6′-(1-fluoro-3-methylbutyl)-6-methyl-[3, 3′-bipyridin]-5-yl)acetate

To a mixture of (S)-isopropyl 2-(5-bromo-4-(4,4-dimethylpiperidin-1-yl)-2-methylpyridin-3-yl)-2-(tert-butoxy)acetate (600 mg, 1.317 mmol), sodium carbonate (279 mg, 2.63 mmol), (6-(1-fluoro-3-methylbutyl)pyridin-3-yl)boronic acid (278 mg, 1.317 mmol) in 1,4-dioxane (30 mL) and water (5.0 mL) was added (Ph₃P)₄Pd (304 mg, 0.263 mmol). The flask was placed under N₂ atm (vac/fill×3) and stirred at 84° C. overnight. Then, EtOAc (400 mL) and H₂O (150 mL) were added, organic layer was separated and washed with H₂O (200 mL×3), dried over Na₂SO₄ and concentrated. The residue was purified on silica gel column (Pet. ether/EtOAc 4:1) to afford the desired product (2S)-isopropyl 2-(tert-butoxy)-2-(4-(4,4-dimethylpiperidin-1-yl)-6′-(1-fluoro-3-methylbutyl)-6-methyl-[3,3′-bipyridin]-5-yl)acetate (400 mg, 35.9% yield). LCMS [M+H]=542.3.

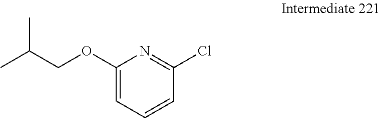

### 2-Chloro-6-isobutoxypyridine

To a solution of 2-methylpropan-1-ol (3.38 g, 45.6 mmol) in DMF (20 mL) was added sodium hydride (1.976 g, 49.4 mmol) and stirred for 20 min. Then, 2-chloro-6-fluoropyridine (5.0 g, 38.0 mmol) was added and stirred at 60° C. overnight. The solution was cooled to room temperature, diluted with 50 mL of EtOAc and the mixture was washed with water (50 mL×2). The organic layer was dried, filtered and concentrated. The crude product was purified by chromatography on silica gel eluting with Pet. ether:EtOAc (10:1) to give 2-chloro-6-isobutoxypyridine (3.96 g, 12.12 mmol, 31.9% yield). LCMS [M+H]=186.

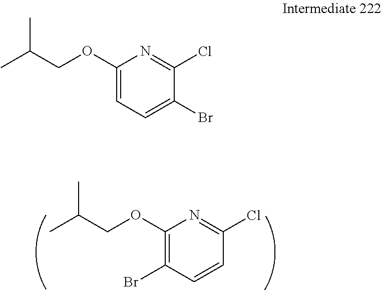

### 3-Bromo-2-chloro-6-isobutoxypyridine

A mixture of 2-chloro-6-isobutoxypyridine (2 g, 10.77 mmol) and 1-bromopyrrolidine-2, 5-dione (2.88 g, 16.16 mmol) in acetonitrile (20 mL) was heated at 85° C. overnight. The solution was evaporated and the residue dissolved in EtOAc (20 m), washed with brine and dried over Na₂SO₄, concentrated in vacuum to afford crude product. The crude product was purified by chromatography (Pet. ether:EtOAc; 100:1) to 3-bromo-2-chloro-6-isobutoxypyridine and 5-bromo-2-chloro-6-isobutoxypyridine mixture (2.6 g, 8.78 mmol, 81% yield). LCMS [M+H]=264.

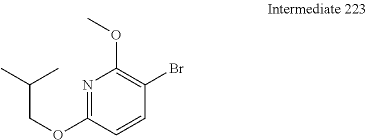

### 3-Bromo-6-isobutoxy-2-methoxypyridine

To a solution of 3-bromo-2-chloro-6-isobutoxypyridine and 5-bromo-2-chloro-6-isobutoxypyridine mixture (2.0 g, 7.56 mmol) in methanol (8 mL) was added sodium methoxide (2.042 g, 37.8 mmol) under an atmosphere of N₂. The mixture was stirred at 100° C. for 4 hr in microwave. The solution was evaporated and the residue dissolved in EtOAc (50 mL), washed with brine solution and dried over Na₂SO₄, concentrated in vacuum to afford crude product. The crude product was purified by silica gel chromatography (Pet. ether:EtOAc; 100:1) to give 3-bromo-6-isobutoxy-2-methoxypyridine (1.1 g, 3.85 mmol, 51% yield). LCMS [M+H]=260 and 261.

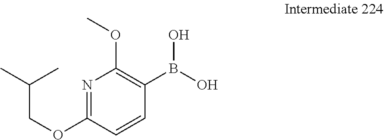 [SMILES: COc1nc(OCC(C)C)ccc1B(O)O]

### (6-Isobutoxy-2-methoxypyridin-3-yl) boronic acid

To a solution of 3-bromo-6-isobutoxy-2-methoxypyridine (500 mg, 1.922 mmol) in THF (20 mL) was added n-butyllithium (1.153 mL, 2.88 mmol) at −78° C. and stirred for 20 min. Then, triisopropyl borate (398 mg, 2.114 mmol) was added and the reaction mixture was stirred at rt for 1 h. The reaction mixture was then diluted with 20 mL of water and neutralized with aq. HCl to pH=6-7. Then, the mixture was extracted with EtOAc (20 mL×2), dried and concentrated. The residue was purified by Prep-TLC (EtOAc:Pet. ether; 1:4) to give (6-isobutoxy-2-methoxypyridin-3-yl) boronic acid (70 mg, 0.229 mmol, 11.91% yield). LCMS [M+H]=226.

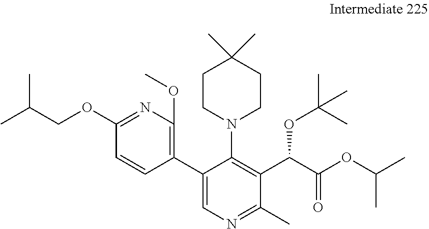 [SMILES: COc1nc(OCC(C)C)ccc1-c1cnc(C)c([C@H](OC(C)(C)C)C(=O)OC(C)C)c1N1CCC(C)(C)CC1]

### (S)-Isopropyl 2-(tert-butoxy)-2-(4-(4, 4-dimethylpiperidin-1-yl)-6′-isobutoxy-2′-methoxy-6-methyl-[3, 3′-bipyridin]-5-yl)acetate

A mixture of (S)-isopropyl 2-(5-bromo-4-(4,4-dimethylpiperidin-1-yl)-2-methylpyridin-3-yl)-2-(tert-butoxy)acetate (100 mg, 0.220 mmol), (6-isobutoxy-2-methoxypyridin-3-yl) boronic acid (59.3 mg, 0.263 mmol) and sodium carbonate (69.8 mg, 0.659 mmol) in 1,4-dioxane (1.5 mL) and water (0.500 mL) was degassed by N₂. Then, the Pd(PPh₃)₄ (50.7 mg, 0.044 mmol) was added and the mixture was heated for 1 h at 110° C. with microwave. Then, the reaction mixture was diluted with 20 mL of water and extracted with EtOAc (20 mL×2). The combined organic layers were dried, concentrated and the resulting residue was purified by Prep-TLC (EtOAc:Pet. ether; 1:3) to give (S)-isopropyl 2-(tert-butoxy)-2-(4-(4,4-dimethylpiperidin-1-yl)-6′-isobutoxy-2′-methoxy-6-methyl-[3,3′-bipyridin]-5-yl)acetate (60 mg, 0.108 mmol, 49.2% yield). LCMS [M+H]=556.4.

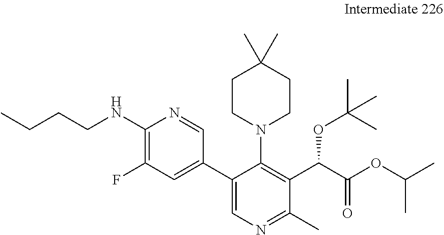 [SMILES: CCCCNc1ncc(-c2cnc(C)c([C@H](OC(C)(C)C)C(=O)OC(C)C)c2N2CCC(C)(C)CC2)cc1F]

### (S)-Isopropyl 2-(tert-butoxy)-2-(6′-(butylamino)-4-(4, 4-dimethylpiperidin-1-yl)-5′-fluoro-6-methyl-[3, 3′-bipyridin]-5-yl)acetate

A mixture of butan-1-amine (1 mL, 10.12 mmol) and (S)-isopropyl 2-(tert-butoxy)-2-(4-(4,4-dimethylpiperidin-1-yl)-5′,6′-difluoro-6-methyl-[3,3′-bipyridin]-5-yl)acetate (70 mg, 0.143 mmol) was sealed in microwave tube and heated at 100° C. for 1 h. Then, the solvent was removed and the residue was purified by Prep-TLC (Pet. ether:EtOAc; 1:1) to afford (S)-isopropyl 2-(tert-butoxy)-2-(6′-(butylamino)-4-(4,4-dimethylpiperidin-1-yl)-5′-fluoro-6-methyl-[3,3′-bipyridin]-5-yl)acetate (70 mg, 0.129 mmol, 90% yield. LCMS=543 [M+H].

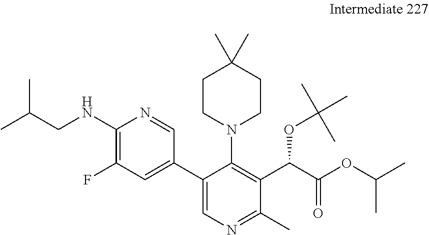 [SMILES: Cc1ncc(-c2cnc(NCC(C)C)c(F)c2)c(N2CCC(C)(C)CC2)c1[C@H](OC(C)(C)C)C(=O)OC(C)C]

### (S)-Isopropyl 2-(tert-butoxy)-2-(4-(4, 4-dimethylpiperidin-1-yl)-5′-fluoro-6′-(isobutyl-amino)-6-methyl-[3, 3′-bipyridin]-5-yl)acetate

A mixture of 2-methylpropan-1-amine (1 mL, 10.12 mmol) and (S)-isopropyl 2-(tert-butoxy)-2-(4-(4,4-dimethylpiperidin-1-yl)-5′,6′-difluoro-6-methyl-[3,3′-bipyridin]-5-yl)acetate (80 mg, 0.163 mmol) was sealed in microwave tube and heated at 100° C. for 1 h. The solvent was removed and the residue was purified by Prep-TLC (Pet. ether:EtOAc; 1:1) to afford (S)-isopropyl 2-(tert-butoxy)-2-(4-(4,4-dimethylpiperidin-1-yl)-5′-fluoro-6′-(isobutylamino)-6-methyl-[3,3′-bipyridin]-5-yl)acetate (70 mg, 0.129 mmol, 79% yield). LCMS m/z=543 [M+H].

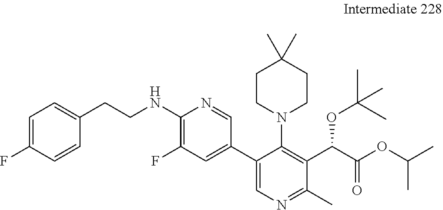 [SMILES: Cc1ncc(-c2cnc(NCCc3ccc(F)cc3)c(F)c2)c(N2CCC(C)(C)CC2)c1[C@H](OC(C)(C)C)C(=O)OC(C)C]

### (S)-Isopropyl 2-(tert-butoxy)-2-(4-(4, 4-dimethylpiperidin-1-yl)-5′-fluoro-6′-((4-fluorophenethyl)amino)-6-methyl-[3, 3′-bipyridin]-5-yl)acetate

A mixture of 2-(4-fluorophenyl)ethanamine (1 mL, 7.62 mmol) and (S)-isopropyl 2-(tert-butoxy)-2-(4-(4,4-dimethylpiperidin-1-yl)-5′,6′-difluoro-6-methyl-[3,3′-bipyridin]-5-yl)acetate (80 mg, 0.163 mmol) was sealed in microwave tube and heated at 100° C. for 1 h. Then, the solvent was removed and the residue was purified by Prep-TLC(Pet. ether:EtOAc; 1:1) afford (S)-isopropyl 2-(tert-butoxy)-2-(4-(4,4-dimethylpiperidin-1-yl)-5′-fluoro-6′-((4-fluorophenethyl)amino)-6-methyl-[3,3′-bipyridin]-5-yl)acetate (80 mg, 0.131 mmol, 80% yield). LCMS=609 [M+H].

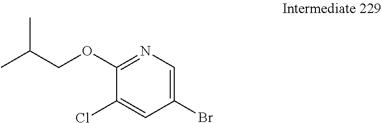

### 5-Bromo-3-chloro-2-isobutoxypyridine

A mixture of 2-methylpropan-1-ol (1.240 g, 16.73 mmol), Cs₂CO₃ (5.95 g, 18.25 mmol) and 5-bromo-3-chloro-2-fluoropyridine (3.2 g, 15.21 mmol) in DMSO (20 mL) was heated at 90° C. overnight. Then, the reaction mixture was taken up in 50 mL of EtOAc and washed with water (50 mL×2). The organic layer was dried, concentrated and the residue was purified by silica gel chromatography to give 5-bromo-3-chloro-2-isobutoxypyridine (3.0 g, 11.34 mmol, 74.6% yield). LCMS=264 [M+H].

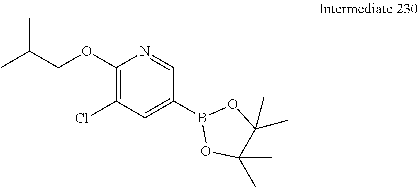 [SMILES: CC(C)COc1ncc(B2OC(C)(C)C(C)(C)O2)cc1Cl]

### 3-Chloro-2-isobutoxy-5-(4,4,5,5-tetramethyl-1,3, 2-dioxaborolan-2-yl)pyridine

A mixture of 1,1′-bis(diphenylphosphino)ferrocenedichloro palladium(II) dichloromethane complex (277 mg, 0.378 mmol), 5-bromo-3-chloro-2-isobutoxypyridine (1000 mg, 3.78 mmol), 4,4,4′,4′,5,5,5′,5′-octamethyl-2,2′-bi(1,3,2-dioxaborolane) (1152 mg, 4.54 mmol) and potassium acetate (1113 mg, 11.34 mmol) in 1,4-dioxane (20 mL) was heated at 90° C. under N₂ atm (wac/fill×3). Then, the reaction mixture was diluted with 50 mL of water and extracted with EtOAc (50 mL×2). The organic layers were dried over Na2SO4, concentrated and the residue was purified by Prep-TLC to give 3-chloro-2-isobutoxy-5-(4,4,5,5-tetramethyl-1,3,2-dioxaborolan-2-yl)pyridine (630 mg, 1.820 mmol, 48.1% yield). LCMS=312 [M+H].

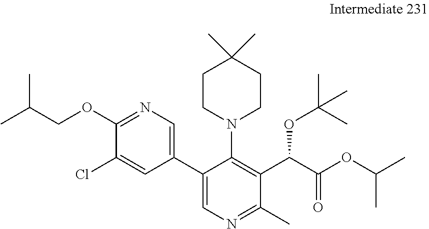 [SMILES: Cc1ncc(-c2cnc(OCC(C)C)c(Cl)c2)c(N2CCC(C)(C)CC2)c1[C@H](OC(C)(C)C)C(=O)OC(C)C]

### (S)-Isopropyl 2-(tert-butoxy)-2-(5′-chloro-4-(4, 4-dimethylpiperidin-1-yl)-6′-isobutoxy-6-methyl-[3, 3′-bipyridin]-5-yl)acetate

A mixture of (S)-isopropyl 2-(5-bromo-4-(4,4-dimethylpiperidin-1-yl)-2-methylpyridin-3-yl)-2-(tert-butoxy)acetate (250 mg, 0.549 mmol), 3-chloro-2-isobutoxy-5-(4,4,5,5-tetramethyl-1,3,2-dioxaborolan-2-yl)pyridine (257 mg, 0.823 mmol), Pd(Ph3P)4 (127 mg, 0.110 mmol) and Na₂CO₃ (175 mg, 1.647 mmol), water (3 mL) and 1,4-dioxane (20 mL) was degassed with N₂ (wac/fill×3) and heated at 90° C. overnight under N2. Then, cooled, diluted with EtOAc (20 ml) and H₂O (20 ml), organic phase separated and aqueous layer extracted with EtOAc (20 ml×3). The combined organic layers were dried with Na₂SO₄ and concentrated. The residue was purified by silica gel column (EtOAc:Pet. ether; 1:5) to afford (S)-isopropyl 2-(tert-butoxy)-2-(5′-chloro-4-(4,4-dimethylpiperidin-1-yl)-6′-isobutoxy-6-methyl-[3,3′-bipyridin]-5-yl)acetate (65 mg, 0.079 mmol, 14.37% yield). LCMS=560 [M+H].

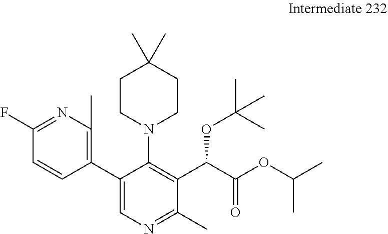 [SMILES: Cc1nc(F)ccc1-c1cnc(C)c([C@H](OC(C)(C)C)C(=O)OC(C)C)c1N1CCC(C)(C)CC1]

### (S)-Isopropyl 2-(tert-butoxy)-2-(4-(4, 4-dimethylpiperidin-1-yl)-6′-fluoro-2′,6-dimethyl-[3, 3′-bipyridin]-5-yl)acetate

A mixture of (S)-isopropyl 2-(5-bromo-4-(4,4-dimethylpiperidin-1-yl)-2-methylpyridin-3-yl)-2-(tert-butoxy)acetate (300 mg, 0.659 mmol), (6-fluoro-2-methylpyridin-3-yl)boronic acid (153 mg, 0.988 mmol), Pd(Ph3P)4 (114 mg, 0.099 mmol) and Na2CO3 (209 mg, 1.976 mmol), water (3 mL) and 1,4-dioxane (20 mL) was degassed with N₂, sealed in microwave tube and heated at 110° C. for 40 min. Then, cooled, diluted with EtOAc (20 ml) and H₂O (50 ml), the organic phase separated and aqueous phase extracted with EtOAc (20 ml×3). The combined organic layers were dried with Na₂SO₄ and concentrated. The residue was purified by silica gel column (EtOAc:Pet. ether; 1:5) to afford (S)-isopropyl 2-(tert-butoxy)-2-(4-(4,4-dimethylpiperidin-1-yl)-6′-fluoro-2′,6-dimethyl-[3,3′-bipyridin]-5-yl)acetate (240 mg, 0.257 mmol, 39.0% yield). LCMS=486 [M+H].

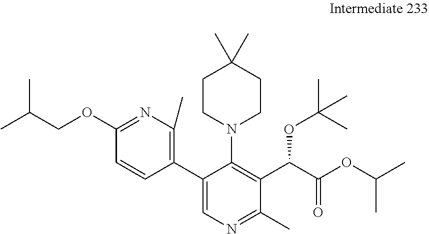 [SMILES: Cc1nc(OCC(C)C)ccc1-c1cnc(C)c([C@H](OC(C)(C)C)C(=O)OC(C)C)c1N1CCC(C)(C)CC1]

### (S)-Isopropyl 2-(tert-butoxy)-2-(4-(4, 4-dimethylpiperidin-1-yl)-6′-isobutoxy-2′,6-dimethyl-[3, 3′-bipyridin]-5-yl)acetate

A mixture of 2-methylpropan-1-ol (145 mg, 1.956 mmol), (S)-isopropyl 2-(tert-butoxy)-2-(4-(4,4-dimethylpiperidin-1-yl)-6′-fluoro-2′,6-dimethyl-[3,3′-bipyridin]-5-yl)acetate (190 mg, 0.391 mmol) and Cs2CO3 (637 mg, 1.956 mmol) in DMF (5 mL) was sealed in microwave tube and heated at 150° C. for 4 h. Then, cooled, diluted with 20 mL of EtOAc and the mixture was washed with water. The organic layer was concentrated and the residue was purified by Prep-TLC to give (S)-isopropyl 2-(tert-butoxy)-2-(4-(4,4-dimethylpiperidin-1-yl)-6′-isobutoxy-2′,6-dimethyl-[3,3′-bipyridin]-5-yl)acetate (37 mg, 0.065 mmol, 16.65% yield). LCMS=540 [M+H].

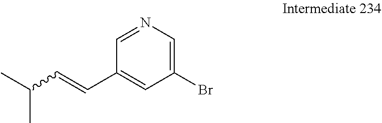

### 3-Bromo-5-(3-methylbut-1-en-1-yl) pyridine

To a mixture of bromo (isobutyl) triphenylphosphorane (8.37 g, 20.97 mmol) in toluene (40 mL) was added potassium 2-methylpropan-2-olate (20.97 mL, 20.97 mmol) at −78° C. and stirred at rt for 4 h. Then, 5-bromonicotinaldehyde (3.0 g, 16.13 mmol) in 10 mL of toluene was added and stirred at rt overnight. Then, the reaction mixture was diluted with 50 ml of water and the mixture was extracted with EtOAc. The combined organic layers were dried, filtered and concentrated. The residue was purified by silica gel chromatography to give 3-bromo-5-(3-methylbut-1-en-1-yl) pyridine (2.85 g, 10.34 mmol, 64.1% yield). LCMS=226 [M+H].

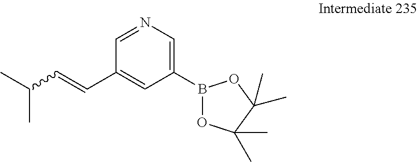 [SMILES: CC(C)C=Cc1cncc(B2OC(C)(C)C(C)(C)O2)c1]

### 3-(3-Methylbut-1-en-1-yl)-5-(4, 4,5,5-tetramethyl-1,3,2-dioxaborolan-2-yl)pyridine

A mixture of 1,1′-bis(diphenylphosphino)ferrocenedichloro palladium(II) dichloromethane complex (324 mg, 0.442 mmol), 3-bromo-5-(3-methylbut-1-en-1-yl)pyridine (1000 mg, 4.42 mmol), 4,4,4′,4′,5,5,5′,5′-octamethyl-2,2′-bi(1,3,2-dioxaborolane) (1685 mg, 6.63 mmol) and KOAc (1302 mg, 13.27 mmol) in 1,4-dioxane (20 mL) was heated at 90° C. under N₂ overnight. Then, cooled, diluted with 30 ml of water and the mixture was extracted with EtOAc (30 mL×3). The combined organic layers were dried, filtered and concentrated. The residue was purified by Prep-TLC (Pet. ether:EtOAc=3:1) to give 3-(3-methylbut-1-en-1-yl)-5-(4,4,5,5-tetramethyl-1,3,2-dioxaborolan-2-yl)pyridine (950 mg, 2.71 mmol, 61.3% yield). LCMS=192 [M-81].

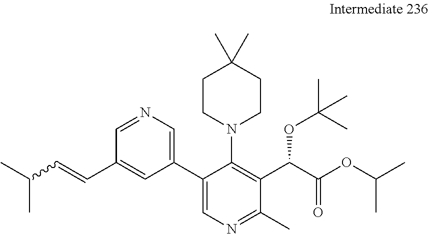 [SMILES: Cc1ncc(-c2cncc(C=CC(C)C)c2)c(N2CCC(C)(C)CC2)c1[C@H](OC(C)(C)C)C(=O)OC(C)C]

### (S)-Isopropyl 2-(tert-butoxy)-2-(4-(4, 4-dimethylpiperidin-1-yl)-6-methyl-5′-(3-methylbut-1-en-1-yl)-[3, 3′-bipyridin]-5-yl)acetate

A stirred mixture of (S)-isopropyl 2-(5-bromo-4-(4,4-dimethylpiperidin-1-yl)-2-methylpyridin-3-yl)-2-(tert-butoxy)acetate (200 mg, 0.439 mmol), 3-(3-methylbut-1-en-1-yl)-5-(4,4,5,5-tetramethyl-1,3,2-dioxaborolan-2-yl)pyridine (180 mg, 0.659 mmol), Pd(Ph3P)4 (507 mg, 0.439 mmol) and Na₂CO₃ (140 mg, 1.317 mmol), water (3 mL) and 1,4-dioxane (10 mL) was placed under N₂ atm (wac/fill×3). The mixture was heated at 90° C. overnight under N₂ and cooled, diluted with EtOAc (50 ml) and H₂O (50 ml). Organic phase separated and aqueous phase extracted with EtOAc (50 ml×3). The combined organic layers were dried with Na₂SO₄ and concentrated. The residue was purified by Prep-TLC (EtOAc:Pet. ether; 1:5) to afford (S)-isopropyl 2-(tert-butoxy)-2-(4-(4,4-dimethylpiperidin-1-yl)-6-methyl-5′-(3-methylbut-1-en-1-yl)-[3,3′-bipyridin]-5-yl)acetate (160 mg, 0.264 mmol, 60.1% yield). LCMS=522 [M+H].

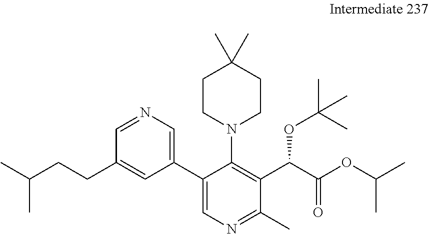 [SMILES: Cc1ncc(-c2cncc(CCC(C)C)c2)c(N2CCC(C)(C)CC2)c1[C@H](OC(C)(C)C)C(=O)OC(C)C]

### (S)-Isopropyl 2-(tert-butoxy)-2-(4-(4, 4-dimethylpiperidin-1-yl)-5′-isopentyl-6-methyl-[3,3′-bipyridin]-5-yl)acetate

A mixture of (S)-isopropyl 2-(tert-butoxy)-2-(4-(4,4-dimethylpiperidin-1-yl)-6-methyl-5′-(3-methylbut-1-en-1-yl)-[3,3′-bipyridin]-5-yl)acetate (80 mg, 0.153 mmol) and Pd/C (15 mg) in methanol (5 mL) was stirred at rt for 1 h under H₂. The mixture was filtered and concentrated to give (S)-isopropyl 2-(tert-butoxy)-2-(4-(4,4-dimethylpiperidin-1-yl)-5′-isopentyl-6-methyl-[3,3′-bipyridin]-5-yl)acetate (70 mg, 0.116 mmol, 76% yield). LCMS=524 [M+H].

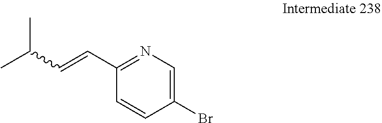

### 5-Bromo-2-(3-methylbut-1-en-1-yl)pyridine

To a mixture of bromo(isobutyl)triphenyl-phosphorane (8.37 g, 20.97 mmol) in toluene (40 mL) was added potassium 2-methylpropan-2-olate (20.97 mL, 20.97 mmol) at −78° C. and stirred at rt for 4 h. Then, 5-bromopicolinaldehyde (3.0 g, 16.13 mmol) in 10 mL of toluene was added and stirred at rt overnight. The reaction mixture was diluted with 50 ml of water and extracted with EtOAc. the organic layer was dried, filtered and concentrated. The residue was purified by silica gel chromatography to give 5-bromo-2-(3-methylbut-1-en-1-yl)pyridine (3.0 g, 8.89 mmol, 55.1% yield. LCMS=226 [M+H].

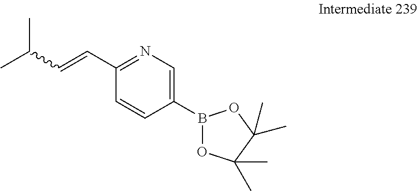 [SMILES: CC(C)C=Cc1ccc(B2OC(C)(C)C(C)(C)O2)cn1]

### 2-(3-Methylbut-1-en-1-yl)-5-(4, 4,5,5-tetramethyl-1,3,2-dioxaborolan-2-yl)pyridine

A mixture of 1,1′-bis(diphenylphosphino)ferrocenedichloro palladium(II) dichloromethane complex (324 mg, 0.442 mmol), (E)-5-bromo-2-(3-methylbut-1-en-1-yl)pyridine (1000 mg, 4.42 mmol), 4,4,4′,4′,5,5,5′,5′-octamethyl-2,2′-bi(1,3,2-dioxaborolane) (1685 mg, 6.63 mmol) and KOAc (1302 mg, 13.27 mmol) in 1,4-dioxane (20 mL) was heated at 90° C. under N₂ overnight. Then, diluted with 30 ml of water and the mixture was extracted with EtOAc (30 mL×3), the combined organic layers were dried, filtered and concentrated. The residue was purified by Prep-TLC (Pet. ether: EtOAc; 3:1) to give 2-(3-methylbut-1-en-1-yl)-5-(4,4,5,5-tetramethyl-1,3,2-dioxaborolan-2-yl)pyridine (950 mg, 1.426 mmol, 32.2% yield). LCMS=192 [M-81].

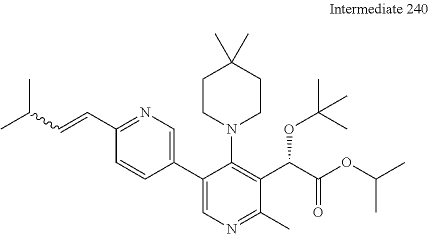 [SMILES: Cc1ncc(-c2ccc(C=CC(C)C)nc2)c(N2CCC(C)(C)CC2)c1[C@H](OC(C)(C)C)C(=O)OC(C)C]

### (S)-Isopropyl 2-(tert-butoxy)-2-(4-(4, 4-dimethylpiperidin-1-yl)-6-methyl-6′-(3-methylbut-1-en-1-yl)-[3, 3′-bipyridin]-5-yl)acetate

A stirred mixture of (S)-isopropyl 2-(5-bromo-4-(4,4-dimethylpiperidin-1-yl)-2-methylpyridin-3-yl)-2-(tert-butoxy)acetate (500 mg, 1.098 mmol), 2-(3-methylbut-1-en-1-yl)-5-(4,4,5,5-tetramethyl-1,3,2-dioxaborolan-2-yl)pyridine (450 mg, 1.647 mmol), Pd(Ph3P)4 (127 mg, 0.110 mmol), Na₂CO₃ (349 mg, 3.29 mmol) water (3 mL) and 1,4-dioxane (10 mL) was placed under N₂ atm (wac/fill×3). Then, the mixture was heated at 90° C. overnight under N2 and, cooled, diluted with EtOAc (50 ml) and H₂O (50 ml). The organic phase separated and aqueous extracted with EtOAc (50 ml×3). The combined organic layers were dried with Na₂SO₄, concentrated and the residue was purified by Prep-TLC (EtOAc:Pet. ether; 1:2) to afford (S)-isopropyl 2-(tert-butoxy)-2-(4-(4,4-dimethylpiperidin-1-yl)-6-methyl-6′-(3-methylbut-1-en-1-yl)-[3,3′-bipyridin]-5-yl)acetate (250 mg, 0.457 mmol, 41.6% yield. LCMS=522.2 [M+H].

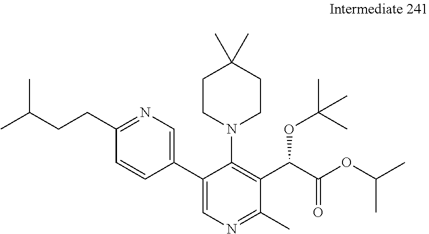 [SMILES: Cc1ncc(-c2ccc(CCC(C)C)nc2)c(N2CCC(C)(C)CC2)c1[C@H](OC(C)(C)C)C(=O)OC(C)C]

### (S)-Isopropyl 2-(tert-butoxy)-2-(4-(4, 4-dimethylpiperidin-1-yl)-6′-isopentyl-6-methyl-[3,3′-bipyridin]-5-yl)acetate

A mixture of (S)-isopropyl 2-(tert-butoxy)-2-(4-(4,4-dimethylpiperidin-1-yl)-6-methyl-6′-(3-methylbut-1-en-1-yl)-[3,3′-bipyridin]-5-yl)acetate (80 mg, 0.153 mmol) and Pd/C (30 mg) in methanol (5 mL) was stirred at rt for 1 h under H2. The mixture was filtered and concentrated to give (S)-isopropyl 2-(tert-butoxy)-2-(4-(4,4-dimethylpiperidin-1-yl)-6′-isopentyl-6-methyl-[3,3′-bipyridin]-5-yl)acetate (75 mg, 0.121 mmol, 79% yield). LCMS=524 [M+H].

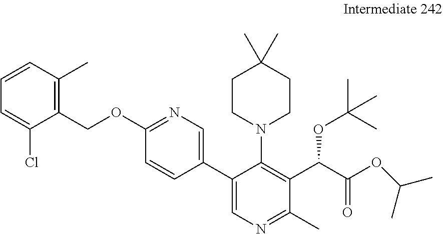 [SMILES: Cc1cccc(Cl)c1COc1ccc(-c2cnc(C)c([C@H](OC(C)(C)C)C(=O)OC(C)C)c2N2CCC(C)(C)CC2)cn1]

### (S)-Isopropyl 2-(tert-butoxy)-2-(6′-((2-chloro-6-methylbenzyl)oxy)-4-(4, 4-dimethylpiperidin-1-yl)-6-methyl-[3, 3′-bipyridin]-5-yl)acetate

To a pre-cooled solution of (2-chloro-6-methylphenyl)methanol (166 mg, 1.060 mmol) in DMF (1 mL) was added the NaH (42.4 mg, 1.060 mmol) in portions. Then, (S)-isopropyl 2-(tert-butoxy)-2-(4-(4,4-dimethylpiperidin-1-yl)-6′-fluoro-6-methyl-[3,3′-bipyridin]-5-yl)acetate (100 mg, 0.212 mmol) was added and the mixture was stirred at room temperature for additional 16 h. The reaction mixture was poured into the ice-water (3 ml). The aqueously layer was extracted with EtOAc (30 ml×3), the combined organic layers were dried over Na₂SO₄ and concentrated to give (S)-isopropyl 2-(tert-butoxy)-2-(6′-((2-chloro-6-methylbenzyl)oxy)-4-(4,4-dimethylpiperidin-1-yl)-6-methyl-[3,3′-bipyridin]-5-yl)acetate as a yellow oil (80 mg, 0.092 mmol, 43.4% yield) which was used in the next step without purification. LCMS [M+H]=609.0.

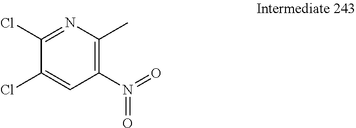

### 2, 3-Dichloro-6-methyl-5-nitropyridine

To a solution of quinoline (1.370 g, 10.61 mmol) was added POCl₃ (1.977 ml, 21.21 mmol) dropwise at 0° C. under N₂ and stirred at 0° C. for 30 min. Then, 3-chloro-6-methyl-5-nitropyridin-2-ol (4 g, 21.21 mmol) was added at 0° C. and the mixture was heated at 120° C. for 2 hr under N₂. Ice water (30 ml) and EtOAc (30 ml) were added to the reaction mixture with stirring. The pH was adjusted to 8 by sat NaHCO₃. The organic layer was washed with brine, dried over N₂SO₄, concentrated to give crude product. The product was purified by flash chromatography (Pet. ether:EtOAc; 20:1) to give 2,3-dichloro-6-methyl-5-nitropyridine (1.7 g, 8.21 mmol, 38.7% yield) as a white solid. LCMS [M+H]=207.0.

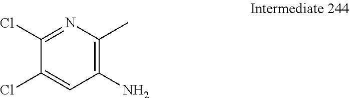

To a solution of 2,3-dichloro-6-methyl-5-nitropyridine (1.7 g, 8.21 mmol) and ammonium chloride (4.39 g, 82 mmol) in ethanol (10 mL) was added iron (2.293 g, 41.1 mmol). The mixture was stirred at 90° C. for 6 hr. Ice water and EtOAc (20 ml each) was added to the reaction mixture with stirring. The organic layer was washed with brine, dried by Na₂SO₄, concentrated to give crude product. The product was purified by Prep-TLC (Pet. ether:EtOAc; 6:1) to give aim product 5,6-dichloro-2-methylpyridin-3-amine (800 mg, 4.52 mmol, 55.0% yield) as a white solid. LCMS [M+H]=177.

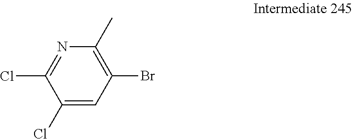

To a solution of 5,6-dichloro-2-methylpyridin-3-amine (850 mg, 4.80 mmol) in HBr (48%, 25 ml) was added dropwise a solution of sodium nitrite (358 mg, 5.19 mmol) in water (8 mL) at 0-5° C. The mixture was stirred at 0-5° C. for 10 min and copper(I) bromide (758 mg, 5.28 mmol) was added at 0° C. Then, the reaction mixture was stirred at rt for 1 h and ice water and EtOAc (30 ml each) were added to the reaction mixture with stirring. The organic layer was washed with brine, dried over Na₂SO₄ and concentrated to give crude product. The product was purified by Prep-TLC (Pet. ether:EtOAc; 20:1) to give desired product 3-bromo-5,6-dichloro-2-methylpyridine (0.6 g, 2.491 mmol, 51.9% yield) as a white solid. LCMS [M+H]=240.0.

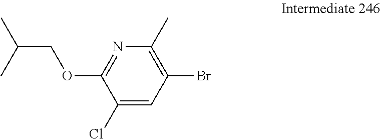

To a solution of 2-methylpropan-1-ol (0.923 g, 12.45 mmol) in DMF (10 mL) was added NaH (0.548 g, 13.70 mmol) at 0-5° C. and stirred at rt for 10 min. Then, 3-bromo-5,6-dichloro-2-methylpyridine (0.6 g, 2.491 mmol) in DMF (2 mL) was added and the mixture was stirred at rt for 16 h. Ice water (20 ml) and EtOAc (20 ml) were added the reaction mixture with stirring. The organic layer was washed with water (20 ml×3), dried over N2SO4, concentrated to give crude product. The product was purified by Pre-TLC (Pet. ether:EtOAc; 20:1) to give desired product 3-bromo-5-chloro-6-isobutoxy-2-methylpyridine (0.4 g, 1.350 mmol, 54.2% yield) as an oil. LCMS [M+H]=278.

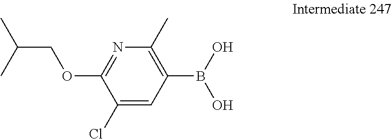 [SMILES: Cc1nc(OCC(C)C)c(Cl)cc1B(O)O]

To a solution of 3-bromo-5-chloro-6-isobutoxy-2-methylpyridine (200 mg, 0.718 mmol) in THF (3 mL) was added n-butyllithium (0.373 mL, 0.933 mmol) at −78° C. and stirred for 20 min at −78° C. Then, triisopropyl borate (270 mg, 1.436 mmol) in THF (2 ml) was added and the reaction mixture was stirred at rt for 1 h. 10 mL of water was added and the mixture was neutralized with aq. HCl to pH=6-7. Then, the mixture was extracted with EtOAc (20 mL×2), dried and concentrated. The residue was purified by Prep TLC (EtOAc:Pet. ether; 1:4) to give (5-chloro-6-isobutoxy-2-methylpyridin-3-yl)boronic acid (80 mg, 0.245 mmol, 34.1% yield) as a white solid. LCMS [M+H]=244.

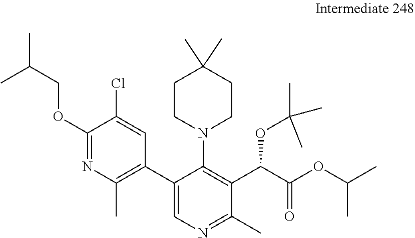 [SMILES: Cc1nc(OCC(C)C)c(Cl)cc1-c1cnc(C)c([C@H](OC(C)(C)C)C(=O)OC(C)C)c1N1CCC(C)(C)CC1]

A mixture of (S)-isopropyl 2-(5-bromo-4-(4,4-dimethylpiperidin-1-yl)-2-methylpyridin-3-yl)-2-(tert-butoxy)acetate (122 mg, 0.267 mmol), (5-chloro-6-isobutoxy-2-methylpyridin-3-yl)boronic acid (65 mg, 0.267 mmol) and Na₂CO₃ (85 mg, 0.801 mmol) in 1,4-dioxane (1.5 mL) and water (0.500 mL) was degassed by N₂. Then, Pd(PPh₃)₄ (61.7 mg, 0.053 mmol) was added and heated for 1 hour at 110° C. with microwave. 20 mL of water was added and the mixture was extracted with EtOAc (20 mL×2), the combined organic layers were dried and concentrated. The residue was purified by Prep-TLC (EtOAc:Pet. ether; 1:3) to give aim product (S)-isopropyl 2-(tert-butoxy)-2-(5′-chloro-4-(4,4-dimethylpiperidin-1-yl)-6′-isobutoxy-2′,6-dimethyl-[3,3′-bipyridin]-5-yl)acetate (20 mg, 0.033 mmol, 12.40% yield) as a white solid. LCMS [M+H]=575.

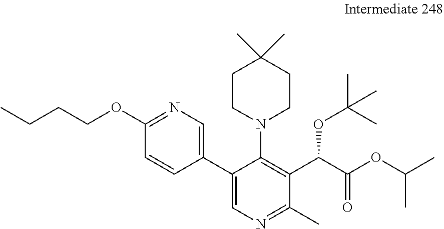 [SMILES: CCCCOc1ccc(-c2cnc(C)c([C@H](OC(C)(C)C)C(=O)OC(C)C)c2N2CCC(C)(C)CC2)cn1]

### (S)-Isopropyl 2-tert-butoxy-2-(6′-butoxy-4-(4, 4-dimethylpiperidin-1-yl)-6-methyl-3, 3′-bipyridin-5-yl)acetate

To a ice-cold solution of butan-1-ol (55.0 mg, 0.742 mmol) in anhydrous THF (3 mL) was added the sodium hydride (29.7 mg, 0.742 mmol). The mixture was stirred at rt for 20 min. Then, (S)-isopropyl 2-tert-butoxy-2-(4-(4,4-dimethylpiperidin-1-yl)-6′-fluoro-6-methyl-3,3′-bipyridin-5-yl)acetate (70 mg, 0.148 mmol) was added and stirred at rt for 2 h. The mixture was poured into the ice-water, extracted with the EtOcAc (25 ml×3), the combined organic layers dried over Na₂SO₄, filtered and concentrated. The crude was purified by Prep-TLC (Pet. ether:EtOAc; 4:1) to give (S)-isopropyl 2-tert-butoxy-2-(6′-butoxy-4-(4,4-dimethylpiperidin-1-yl)-6-methyl-3,3′-bipyridin-5-yl)acetate (55 mg, 58%). LCMS [M+H]=526.4.

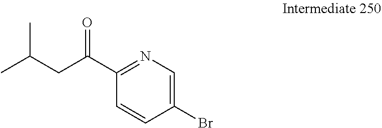

### 1-(5-Bromopyridin-2-yl)-3-methylbutan-1-one

To a ice-cold solution of 1-(5-bromopyridin-2-yl)-3-methylbutan-1-ol (3.35 g, 13.72 mmol) in dichloromethane (70 mL) was added Dess-martinperiodinane (11.64 g, 27.4 mmol). The mixture was stirred for 4 h at 25° C. Then, NaHCO₃ sat. (100 ml) was added, extracted with EA (200 ml×3). The combined organic phase were dried over Na₂SO₄ and concentrated. The residue was purified on silica gel column (PE/EA 20:1) to afford desired product 1-(5-bromopyridin-2-yl)-3-methylbutan-1-one (3.3 g, 13.49 mmol, 98% yield) yellow oil. LCMS [M+H]=242.1.

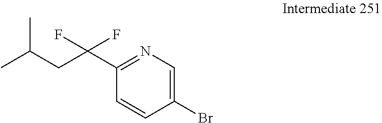

### 5-Bromo-2-(1,1-difluoro-3-methylbutyl)pyridine

To a ice-cold 1-(5-bromopyridin-2-yl)-3-methylbutan-1-one (3.3 g, 13.63 mmol) was added Bast (10.05 ml, 54.5 mmol). The mixture was stirred overnight at 60° C. Then, cooled, diluted with 5 M K₃PO₄ (100 ml) and extracted with EA (150 ml×3). The combined organic layers were dried over Na₂SO₄ and concentrated. The residue was purified by Prep-HPLC to afford desired product 5-bromo-2-(1,1-difluoro-3-methylbutyl)pyridine (600 mg, 2.272 mmol, 16.67% yield) as yellow oil. LCMS [M+H]=264.2.

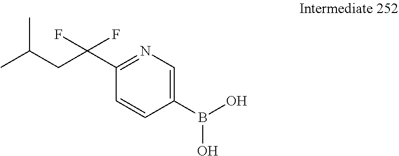 [SMILES: CC(C)CC(F)(F)c1ccc(B(O)O)cn1]

### 6-(1,1-Difluoro-3-methylbutyl)pyridin-3-ylboronic acid

A mixture of 5-bromo-2-(1,1-difluoro-3-methylbutyl)pyridine (300 mg, 1.136 mmol), 4,4,4′,4′,5,5,5′,5′-octamethyl-2,2′-bi(1,3,2-dioxaborolane) (433 mg, 1.704 mmol), potassium acetate (334 mg, 3.41 mmol) and PdCl2(dppf) (166 mg, 0.227 mmol) in 1,4-dioxane(10 ml) was heated at 100° C. for 16 h under N₂ atmosphere. The mixture was then filtered, concentrated, the residue taken up in water and extracted with the EtOAc (70 ml×3). The combined organic layers were dried over NaSO4, concentrated and purified by prep-TLC(Pet. ether:EtOAc; 5:1) to give 6-(1,1-difluoro-3-methylbutyl)pyridin-3-ylboronic acid (160 mg, 29%). LCMS [M+H]=230.1.

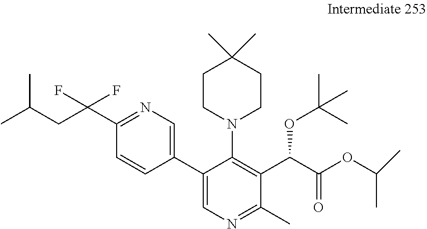 [SMILES: Cc1ncc(-c2ccc(C(F)(F)CC(C)C)nc2)c(N2CCC(C)(C)CC2)c1[C@H](OC(C)(C)C)C(=O)OC(C)C]

### (S)-Isopropyl 2-tert-butoxy-2-(6′-(1,1-difluoro-3-methylbutyl)-4-(4, 4-dimethylpiperidin-1-yl)-6-methyl-3, 3′-bipyridin-5-yl)acetate

A mixture of (S)-isopropyl 2-(5-bromo-4-(4,4-dimethylpiperidin-1-yl)-2-methylpyridin-3-yl)-2-(tert-butoxy)acetate (100 mg, 0.220 mmol), (6-(1,1-difluoro-3-methylbutyl)pyridin-3-yl)boronic acid (101 mg, 0.439 mmol), Na2CO3 (69.8 mg, 0.659 mmol) in 1,4-dioxane (3 ml) and water (1 ml) was degassed with N2. Then, Pd(PPh3)4(50.7 mg, 0.044 mmol) was added and heated at 110° C. for 16 h under N2 atmosphere. The mixture was then filtered, concentrated, the residue was taken up into water and extracted with the EA (50 ml×3). The combined organic layers were dried over NaSO4, concentrated and purified by prep-TLC (Pet. ether:EtOAc; 6:1) to give (S)-isopropyl 2-tert-butoxy-2-(6′-(1,1-difluoro-3-methylbutyl)-4-(4,4-dimethylpiperidin-1-yl)-6-methyl-3,3′-bipyridin-5-yl)acetate (70 mg, 57%). LCMS [M+H]=560.3.

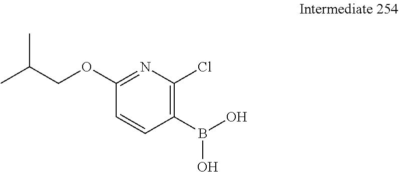

### (2-Chloro-6-isobutoxypyridin-3-yl)boronic acid

To a solution of 3-bromo-2-chloro-6-isobutoxypyridine (1.4 g, 5.29 mmol) in THF (20 mL) was added n-butyllithium (3.18 mL, 7.94 mmol) at −78° C. and stirred for 20 min at −78°. Then, triisopropyl borate (1.095 g, 5.82 mmol) was added and the reaction mixture was stirred at rt for 1 h. The reaction mixture was diluted with water (20 mL), neutralized with aq. HCl to pH=6-7 and extracted with EtOAc (20 mL×2). The combined organic layers were dried over Na2SO— and concentrated. The residue was purified by Prep_TLC (EtOAc:Pet. Ether=1:3) to give (2-chloro-6-isobutoxypyridin-3-yl)boronic acid (450 mg, 1.902 mmol, 35.9% yield) as white solid. LCMS [M+H]=230.

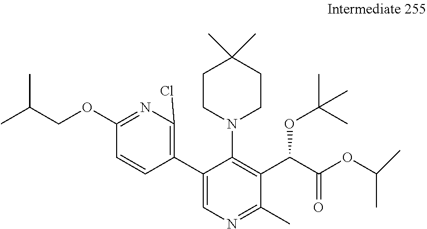 [SMILES: Cc1ncc(-c2ccc(OCC(C)C)nc2Cl)c(N2CCC(C)(C)CC2)c1[C@H](OC(C)(C)C)C(=O)OC(C)C]

### (2S)-Isopropyl 2-tert-butoxy-2-(2′-chloro-4-(4, 4-dimethylpiperidin-1-yl)-6′-isobutoxy-6-methyl-3, 3′-bipyridin-5-yl)acetate

The solution of (S)-isopropyl 2-(5-bromo-4-(4,4-dimethylpiperidin-1-yl)-2-methylpyridin-3-yl)-2-tert-butoxyacetate (200 mg, 0.439 mmol), 2-chloro-6-isobutoxypyridin-3-ylboronic acid (202 mg, 0.878 mmol) and sodium carbonate (140 mg, 1.317 mmol) in 1,4-dioxane (3 ml) and water (1 ml) was degassed by N2. Then, Pd(PPh3)4 (101 mg, 0.088 mmol) was added and heated at 110° C. for 1 h in microwave. The mixture was filtered, concentrated, taken up into water and extracted with the EtOAc (50 ml×3). The combined organic layers were dried over NaSO4, concentrated and the crude was purified by prep-TLC (Pet. ether:EtOAc; 3:1) to give (2S)-isopropyl 2-tert-butoxy-2-(2′-chloro-4-(4,4-dimethylpiperidin-1-yl)-6′-isobutoxy-6-methyl-3,3′-bipyridin-5-yl)acetate (100 mg, 27%). LCMS [M+H]=560.3.

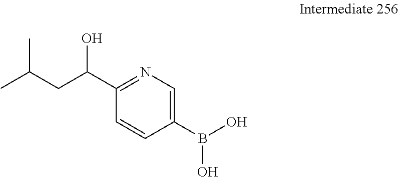

### 6-(1-Hydroxy-3-methylbutyl)pyridin-3-ylboronic acid

A mixture of 1-(5-bromopyridin-2-yl)-3-methylbutan-1-ol (800 mg, 3.28 mmol), 4,4,4′,4′,5,5,5′,5′-octamethyl-2,2′-bi(1,3,2-dioxaborolane) (1248 mg, 4.92 mmol), potassium acetate (965 mg, 9.83 mmol) and PdCl2(dppf) (480 mg, 0.655 mmol) in 1,4-dioxane (10 ml) was heated at 100° C. for 16 h under N2 atmosphere. The mixture was used in the next step without purification. LCMS [M+H]=210.0.

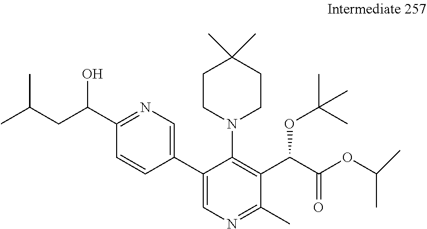 [SMILES: Cc1ncc(-c2ccc(C(O)CC(C)C)nc2)c(N2CCC(C)(C)CC2)c1[C@H](OC(C)(C)C)C(=O)OC(C)C]

### (2S)-Isopropyl 2-tert-butoxy-2-(4-(4, 4-dimethylpiperidin-1-yl)-6′-(1-hydroxy-3-methylbutyl)-6-methyl-3, 3′-bipyridin-5-yl)acetate

The mixture of (S)-isopropyl 2-(5-bromo-4-(4,4-dimethylpiperidin-1-yl)-2-methylpyridin-3-yl)-2-(tert-butoxy)acetate (600 mg, 1.32 mmol), 6-(1-hydroxy-3-methylbutyl)pyridin-3-ylboronic acid (crude from previous reaction, 3.83 mmol), Na2CO3 (1217 mg, 11.48 mmol) in 1,4-dioxane (5 ml) and water (1.5 ml) was degassed with N2. Then, Pd(Ph3P)4 (884 mg, 0.765 mmol) was added and heated at 110° C. for 16 h under N2 atmosphere. The mixture filtered, concentrated and the residue was taken up into water and extracted with EtOAc (80 ml×3). The combined organic layers were dried over NaSO4, concentrated and purified by prep-TLC (Pet. ether:EtOAc; 1:1) to give (2S)-isopropyl 2-tert-butoxy-2-(4-(4,4-dimethylpiperidin-1-yl)-6′-(1-hydroxy-3-methylbutyl)-6-methyl-3,3′-bipyridin-5-yl)acetate (250 mg, 35%). LCMS [M+H]=540.3.

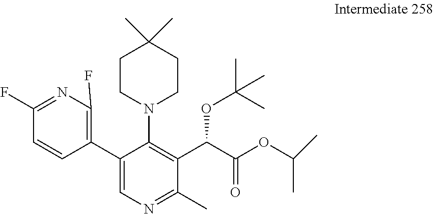 [SMILES: Cc1ncc(-c2ccc(F)nc2F)c(N2CCC(C)(C)CC2)c1[C@H](OC(C)(C)C)C(=O)OC(C)C]

A mixture of (S)-isopropyl 2-(5-bromo-4-(4,4-dimethylpiperidin-1-yl)-2-methylpyridin-3-yl)-2-tert-butoxyacetate (400 mg, 0.878 mmol), 2,6-difluoropyridin-3-ylboronic acid (279 mg, 1.757 mmol) and sodium carbonate (279 mg, 2.63 mmol) in 1,4-dioxane (6 ml) and water (2 ml) was degassed by bubbling N₂ through the reaction mixture. Then, the Pd(PPh₃)₄ (203 mg, 0.176 mmol) was added and the mixture was heated at 110° C. for 1 h in microwave. Then, the mixture was filtered and the filtrate was concentrate. The residue was suspended in water and extracted with the EtOAc (3×50 mL). The combined organic layers were dried over Na₂SO₄ and concentrated. The crude product was purified by prep-TLC (Pet. ether:EtOAc=2:1), to give (2S)-isopropyl 2-tert-butoxy-2-(4-(4,4-dimethylpiperidin-1-yl)-2′,6′-difluoro-6-methyl-3,3′-bipyridin-5-yl)acetate (210 mg, 24%). LCMS [M+H]=490.2.

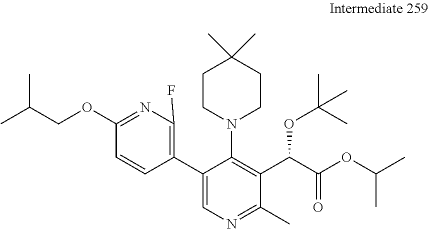 [SMILES: Cc1ncc(-c2ccc(OCC(C)C)nc2F)c(N2CCC(C)(C)CC2)c1[C@H](OC(C)(C)C)C(=O)OC(C)C]

### (2S)-Isopropyl 2-tert-butoxy-2-(4-(4, 4-dimethylpiperidin-1-yl)-2′-fluoro-6′-isobutoxy-6-methyl-3, 3′-bipyridin-5-yl)acetate

To a pre-cooled solution of 2-methylpropan-1-ol (32 mg, 0.43 mmol) in anhydrous DMF (3 mL) was added the NaH (17 mg, 0.43 mmol) and the mixture was stirred at this temperature for 30 min. Then, (2S)-isopropyl 2-tert-butoxy-2-(4-(4,4-dimethylpiperidin-1-yl)-2′,6′-difluoro-6-methyl-3,3′-bipyridin-5-yl)acetate (210 mg, 0.43 mmol) was added. The mixture was stirred at rt overnight and poured into ice-water, extracted with the EtOAc (50 ml×3), dried over Na2SO4 and concentrated. The crude was purified by prep-HPLC to give (2S)-isopropyl 2-tert-butoxy-2-(4-(4,4-dimethylpiperidin-1-yl)-2′-fluoro-6′-isobutoxy-6-methyl-3,3′-bipyridin-5-yl)acetate (51 mg, 17%). LCMS [M+H]=544.4.

Also isolated

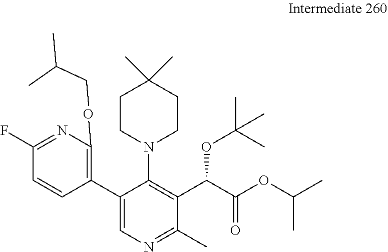 [SMILES: Cc1ncc(-c2ccc(F)nc2OCC(C)C)c(N2CCC(C)(C)CC2)c1[C@H](OC(C)(C)C)C(=O)OC(C)C]

(2S)-Isopropyl 2-tert-butoxy-2-(4-(4, 4-dimethylpiperidin-1-yl)-6′-fluoro-2′-isobutoxy-6-methyl-3,3′-bipyridin-5-yl)acetate: (30 mg, 38%). LCMS [M+H]=544.4.

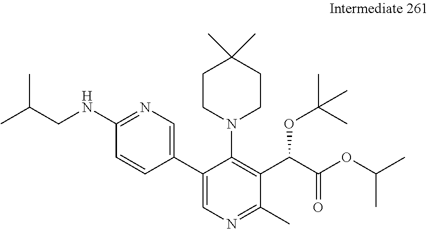 [SMILES: Cc1ncc(-c2ccc(NCC(C)C)nc2)c(N2CCC(C)(C)CC2)c1[C@H](OC(C)(C)C)C(=O)OC(C)C]

### (S)-Isopropyl 2-tert-butoxy-2-(4-(4, 4-dimethylpiperidin-1-yl)-6′-(isobutylamino)-6-methyl-3, 3′-bipyridin-5-yl)acetate

A mixture of (S)-isopropyl 2-(tert-butoxy)-2-(4-(4,4-dimethylpiperidin-1-yl)-6′-fluoro-6-methyl-[3,3′-bipyridin]-5-yl)acetate (100 mg, 0.212 mmol) and 2-methylpropan-1-amine (736 mg, 10.06 mmol) in THF (5 mL) was stirred at 100° C. for 16 hours. Then, the reaction was diluted with water (20 ml) and extracted with EtOAc (2×40 mL). The combined organic layers were washed with brine (20 mL), dried over Na₂SO₄, concentrated to afford a crude product as an oil. The crude product was purified on silica gel column (Pet. ether:EtOAc=4:1) to afford (S)-isopropyl 2-tert-butoxy-2-(4-(4,4-dimethylpiperidin-1-yl)-6′-(isobutylamino)-6-methyl-3,3′-bipyridin-5-yl)acetate (55 mg, 41.48%). LCMS [M+H]=525.4.

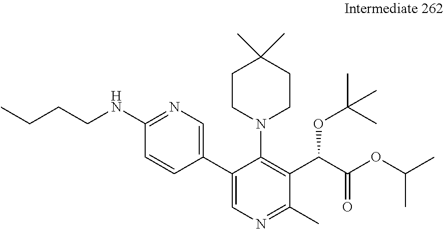 [SMILES: CCCCNc1ccc(-c2cnc(C)c([C@H](OC(C)(C)C)C(=O)OC(C)C)c2N2CCC(C)(C)CC2)cn1]

### (S)-Isopropyl 2-tert-butoxy-2-(6′-(butylamino)-4-(4, 4-dimethylpiperidin-1-yl)-6-methyl-3,3′-bipyridin-5-yl)acetate

A mixture of (S)-isopropyl 2-(tert-butoxy)-2-(4-(4,4-dimethylpiperidin-1-yl)-6′-fluoro-6-methyl-[3,3′-bipyridin]-5-yl)acetate (70 mg, 0.148 mmol) and butan-1-amine (217 mg, 2.97 mmol) in THF (5 mL) was stirred at 100° C. for 16 hours. Then, the mixture was diluted with water (20 mL) and extracted with EtOAc (2×40 mL). The combined organic layers were washed with brine (20 mL), dried over Na₂SO₄, and concentrated to afford (S)-isopropyl 2-tert-butoxy-2-(6′-(butylamino)-4-(4,4-dimethylpiperidin-1-yl)-6-methyl-3,3′-bipyridin-5-yl)acetate (55 mg, 29.3%). LCMS [M+H]=525.0.

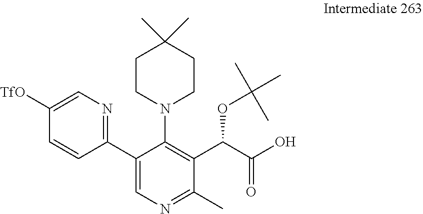 [SMILES: Cc1ncc(-c2ccc(OS(=O)(=O)C(F)(F)F)cn2)c(N2CCC(C)(C)CC2)c1[C@H](OC(C)(C)C)C(=O)O]

### (S)-Isopropyl 2-(tert-butoxy)-2-(4′-(4, 4-dimethylpiperidin-1-yl)-6′-methyl-5-(((trifluoromethyl)sulfonyl)oxy)-[2, 3′-bipyridin]-5′-yl)acetate

To a mixture of (S)-isopropyl 2-(tert-butoxy)-2-(4′-(4,4-dimethylpiperidin-1-yl)-5-hydroxy-6′-methyl-[2,3′-bipyridin]-5′-yl)acetate (500 mg, 1.065 mmol) and triethylamine (215 mg, 2.129 mmol) in dichloromethane (8 mL) was added 1,1,1-trifluoro-N-(pyridin-2-yl)-N-((trifluoromethyl)-sulfonyl)methanesulfonamide (496 mg, 1.384 mmol) at 0° C. and stirred at rt for 2 h. Then, the reaction mixture was diluted with water (20 mL) and extracted with DCM (20 mL×2). The combined organic layers were dried over Na2SO4, filtered and concentrated. The residue was purified by Prep-TLC (Pet. ether:EtOAc=5:1) to afford (S)-isopropyl 2-(tert-butoxy)-2-(4′-(4,4-dimethylpiperidin-1-yl)-6′-methyl-5-(((trifluoromethyl)sulfonyl)-oxy)-[2,3′-bipyridin]-5′-yl)acetate (580 mg, 0.954 mmol, 90% yield as syrup. LCMS [M+H]=602.

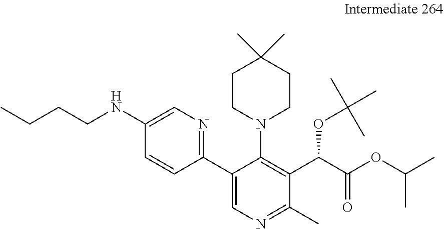 [SMILES: CCCCNc1ccc(-c2cnc(C)c([C@H](OC(C)(C)C)C(=O)OC(C)C)c2N2CCC(C)(C)CC2)nc1]

### (S)-Isopropyl 2-(tert-butoxy)-2-(5-(butylamino)-4′-(4, 4-dimethylpiperidin-1-yl)-6′-methyl-[2, 3′-bipyridin]-5′-yl)acetate

A stirred mixture of tris(dibenzylideneacetone)di-palladium(0) (22.83 mg, 0.025 mmol), (S)-isopropyl 2-(tert-butoxy)-2-(4′-(4,4-dimethylpiperidin-1-yl)-6′-methyl-5-(((trifluoromethyl)sulfonyl)oxy)-[2,3′-bipyridin]-5′-yl)acetate (150 mg, 0.249 mmol), (9,9-dimethyl-9H-xanthene-4,5-diyl)bis(diphenyl-phosphine) (28.9 mg, 0.050 mmol), butan-1-amine (36.5 mg, 0.499 mmol) and Cs2CO3 (244 mg, 0.748 mmol) in toluene (10 mL) was placed under N₂ atm (wac/fill×3). The mixture was heated at 100° C. overnight under N2. Then, diluted with EtOAc (50 ml) and H₂O (20 ml) and extracted with EtOAc (50 ml×3). The combined organic layers were dried over Na₂SO₄ and concentrated. The residue was purified by Prep-TLC (Pet. ether:EtOAc=1:1) to give (S)-isopropyl 2-(tert-butoxy)-2-(5-(butylamino)-4′-(4,4-dimethylpiperidin-1-yl)-6′-methyl-[2,3′-bipyridin]-5′-yl)acetate (65 mg, 0.105 mmol, 42.2% yield). LCMS [M+H]=525.

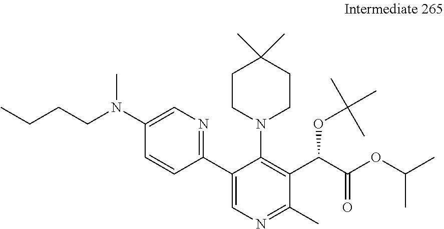 [SMILES: CCCCN(C)c1ccc(-c2cnc(C)c([C@H](OC(C)(C)C)C(=O)OC(C)C)c2N2CCC(C)(C)CC2)nc1]

### (S)-Isopropyl 2-(tert-butoxy)-2-(5-(butylamino)-4′-(4, 4-dimethylpiperidin-1-yl)-6′-methyl-[2, 3′-bipyridin]-5′-yl)acetate

A stirred mixture of tris(dibenzylideneacetone)-dipalladium(0) (22.83 mg, 0.025 mmol), (S)-isopropyl 2-(tert-butoxy)-2-(4′-(4,4-dimethyl-piperidin-1-yl)-6′-methyl-5-(((trifluoromethyl)sulfonyl)oxy)-[2,3′-bipyridin]-5′-yl)acetate (150 mg, 0.249 mmol), (9,9-dimethyl-9H-xanthene-4,5-diyl)bis(diphenylphosphine) (28.9 mg, 0.050 mmol), N-methylbutan-1-amine (43.5 mg, 0.499 mmol) and Cs2CO3 (244 mg, 0.748 mmol) in toluene (10 mL) was placed under N₂ atm (wac/fill×3). The mixture was heated at 100° C. overnight under N2. Then, diluted EtOAc (50 ml) and H₂O (20 ml) and extracted with EtOAc (50 ml×3). The combined organic layers were dried with Na₂SO₄ and concentrated. The residue was purified by Prep-TLC (Pet. ether:EtOAc=1:1) to give (S)-isopropyl 2-(tert-butoxy)-2-(5-(butylamino)-4′-(4,4-dimethylpiperidin-1-yl)-6′-methyl-[2,3′-bipyridin]-5′-yl)acetate (50 mg, 0.094 mmol, 37.6% yield). LCMS [M+H]=539.

### Example 1

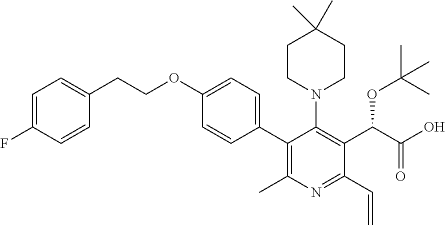 [SMILES: C=Cc1nc(C)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-6-methyl-2-vinylpyridin-3-yl)acetic acid

To a solution of (S)-isopropyl 2-(tert-butoxy)-2-(4-(4,4-dimethylpiperidin-1-yl)-5-(4-(4-fluorophenethoxy)phenyl)-6-methyl-2-vinylpyridin-3-yl)acetate (11 mg, 0.018 mmol) in EtOH (1 mL) and water (0.111 mL) was added lithium hydroxide monohydrate (7.48 mg, 0.178 mmol) and heated at 75° C. for 48 hrs. The reaction was cooled to RT, filtered through a nylon 0.45 g frit filter and purified via preparative LC/MS to afford the product (S)-2-(tert-butoxy)-2-(4-(4,4-dimethylpiperidin-1-yl)-5-(4-(4-fluorophenethoxy)phenyl)-6-methyl-2-vinylpyridin-3-yl)acetic acid (7.3 mg, 0.013 mmol, 71.2% yield). ¹H NMR (500 MHz, DMSO-d6) δ 7.38 (dd, J=8.2, 5.8 Hz, 2H), 7.27-7.20 (m, 2H), 7.18-7.12 (m, 2H), 7.08-7.01 (m, 2H), 6.26 (dd, J=16.9, 2.9 Hz, 1H), 5.92 (br s, 1H), 5.30 (dd, J=10.8, 2.6 Hz, 1H), 4.29-4.19 (m, 2H), 3.06 (t, J=6.7 Hz, 2H), 2.23-2.16 (m, 1H), 2.11 (s, 3H), 1.96 (br t, J=11.6 Hz, 1H), 1.54-1.44 (m, 1H), 1.34-1.17 (m, 1H), 1.12 (s, 9H), 1.07-0.99 (m, 2H), 0.85 (s, 3H), 0.60 (s, 3H) 3 protons on the piperidine ring and the 2 protons of the methylene on the 4-fluorophenethoxy were not resolved via ¹H NMR due to water in the experiment. LCMS (M+1)=575.2.

### Example 2

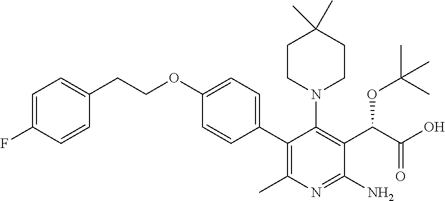 [SMILES: Cc1nc(N)c([C@H](OC(C)(C)C)C(=O)O)c(N2CCC(C)(C)CC2)c1-c1ccc(OCCc2ccc(F)cc2)cc1]

### (S)-2-(2-Amino-4-(4, 4-dimethylpiperidin-1-yl)-5-(4-(4-fluorophenethoxy)phenyl)-6-methylpyridin-3-yl)-2-(tert-butoxy)acetic acid

To a solution of (S)-isopropyl 2-(2-amino-4-(4,4-dimethylpiperidin-1-yl)-5-(4-(4-fluorophenethoxy)phenyl)-6-methylpyridin-3-yl)-2-(tert-butoxy)acetate (20 mg, 0.033 mmol) in EtOH (1 mL) and water (0.111 mL) was added lithium hydroxide monohydrate (13.85 mg, 0.330 mmol) and heated at 75° C. for 2 hrs. The reaction was cooled to RT, filtered through a nylon 0.45 g frit filter and purified via preparative LC/MS to afford the product (S)-2-(2-amino-4-(4,4-dimethylpiperidin-1-yl)-5-(4-(4-fluorophenethoxy)phenyl)-6-methylpyridin-3-yl)-2-(tert-butoxy)acetic acid (1.8 mg, 2.94 mol, 8.90% yield). ¹H NMR (500 MHz, DMSO-d₆) δ 7.37 (dd, J=8.4, 5.9 Hz, 2H), 7.19-7.11 (m, 3H), 7.08-6.96 (m, 3H), 5.81-5.70 (m, 1H), 4.27-4.15 (m, 2H), 3.50-3.34 (m, 1H), 3.04 (t, J=7.0 Hz, 2H), 1.96-1.87 (m, 3H), 1.29-1.18 (m, 2H), 1.16 (s, 9H), 0.90-0.54 (m, 6H). 5 protons on the piperidine ring were not resolved via ¹H NMR due to water suppression in the experiment. LCMS (M+1)=564.2.

### Example 3 Example 4

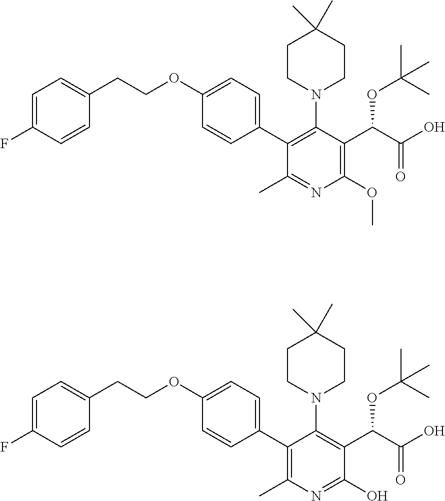 [SMILES: COc1nc(C)c(-c2ccc(OCCc3ccc(F)cc3)cc2)c(N2CCC(C)(C)CC2)c1[C@H](OC(C)(C)C)C(=O)O ; Cc1nc(O)c([C@H](OC(C)(C)C)C(=O)O)c(N2CCC(C)(C)CC2)c1-c1ccc(OCCc2ccc(F)cc2)cc1]

### (S)-2-(tert-Butoxy)-2-(4-(4, 4-dimethylpiperidin-1-yl)-5-(4-(4-fluorophenethoxy)phenyl)-2-methoxy-6-methylpyridin-3-yl)acetic acid and (S)-2-(tert-butoxy)-2-(4-(4, 4-dimethylpiperidin-1-yl)-5-(4-(4-fluorophenethoxy)phenyl)-6-methyl-2-oxo-1, 2-dihydropyridin-3-yl)acetic acid

(S)-Isopropyl 2-(2-amino-4-(4,4-dimethylpiperidin-1-yl)-5-(4-(4-fluorophenethoxy)phenyl)-6-methylpyridin-3-yl)-2-(tert-butoxy)acetate (50 mg, 0.083 mmol) and copper(II) chloride (33.3 mg, 0.248 mmol) were combined in 37% HCl (413 μl) and MeOH (413 μl) in a 7 ml vial. The mixture was then cooled to 0° C. and a solution of sodium nitrite (17.08 mg, 0.248 mmol) in 0.1 mL H₂O was added. The vial was sealed and the mixture was warmed to room temp and stirred for 5 h. The mixture was diluted with EtOAc and washed with water, brine, dried (MgSO₄), filtered and concentrated. The residue was taken up in EtOH (1 mL) and water (0.1 mL) and added lithium hydroxide monohydrate (34.6 mg, 0.825 mmol). The reaction was heated to 75° C. for 18 hrs. The reaction was cooled to RT and filtered through a 0.45 g nylon frit filter and purified via preparative LC/MS to afford the products (S)-2-(tert-butoxy)-2-(4-(4,4-dimethylpiperidin-1-yl)-5-(4-(4-fluorophenethoxy)phenyl)-2-methoxy-6-methylpyridin-3-yl)acetic acid (2.6 mg, 4.49 μmol, 5.44% yield) and (S)-2-(tert-butoxy)-2-(4-(4,4-dimethylpiperidin-1-yl)-5-(4-(4-fluorophenethoxy)phenyl)-6-methyl-2-oxo-1,2-dihydropyridin-3-yl)acetic acid (6.8 mg, 0.012 mmol, 14.59% yield). (S)-2-(tert-Butoxy)-2-(4-(4,4-dimethylpiperidin-1-yl)-5-(4-(4-fluorophenethoxy)phenyl)-2-methoxy-6-methylpyridin-3-yl)acetic acid ¹H NMR (500 MHz, DMSO-d6) δ 7.36 (dd, J=8.4, 5.5 Hz, 2H), 7.17-7.10 (m, 3H), 7.07-7.03 (m, 1H), 7.02-6.96 (m, 2H), 5.35 (s, 1H), 4.27-4.16 (m, 2H), 3.82 (s, 3H), 3.62-3.45 (m, 1H), 3.03 (t, J=6.6 Hz, 2H), 2.86-2.68 (m, 1H), 2.24-2.07 (m, 1H), 2.01 (s, 3H), 1.71-1.54 (m, 1H), 1.14 (s, 9H), 0.82-0.75 (m, 3H), 0.54 (br s, 3H). 4 protons on the piperidine ring were not resolved via ¹H NMR due to water suppression in the experiment. LCMS (M+1)=579.1.

(S)-2-(tert-Butoxy)-2-(4-(4,4-dimethylpiperidin-1-yl)-5-(4-(4-fluorophenethoxy)phenyl)-6-methyl-2-oxo-1,2-dihydropyridin-3-yl)acetic acid ¹H NMR (500 MHz, DMSO-d6) δ 7.35 (dd, J=8.3, 5.7 Hz, 1H), 7.19-7.15 (m, 1H), 7.12 (t, J=8.8 Hz, 2H), 7.07-7.01 (m, 1H), 6.97 (brt, J=7.3 Hz, 2H), 5.36-5.25 (m, 1H), 4.25-4.14 (m, 2H), 3.59-3.49 (m, 1H), 3.02 (t, J=6.6 Hz, 2H), 2.55 (s, 3H), 1.92-1.82 (m, 4H), 1.14 (s, 9H), 0.78-0.45 (m, 6H). 4 protons on the piperidine ring were not resolved via ¹H NMR due to water suppression in the experiment. LCMS (M+1)=565.2.

### Example 5

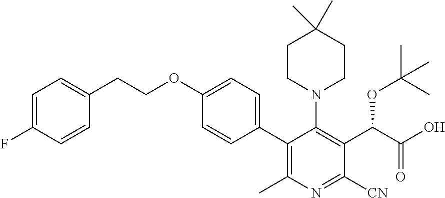 [SMILES: Cc1nc(C#N)c([C@H](OC(C)(C)C)C(=O)O)c(N2CCC(C)(C)CC2)c1-c1ccc(OCCc2ccc(F)cc2)cc1]

### (S)-2-(tert-Butoxy)-2-(2-cyano-4-(4, 4-dimethylpiperidin-1-yl)-5-(4-(4-fluorophenethoxy)phenyl)-6-methylpyridin-3-yl)acetic acid

To a solution of (S)-isopropyl 2-(tert-butoxy)-2-(2-cyano-4-(4,4-dimethylpiperidin-1-yl)-5-(4-(4-fluorophenethoxy)phenyl)-6-methylpyridin-3-yl)acetate (20 mg, 0.032 mmol) in EtOH (1 ml) and water (0.111 ml) was added lithium hydroxide monohydrate (2.73 mg, 0.065 mmol) and heated at 75° C. for 30 min. After 30 minutes, the LCMS indicated the reaction was complete. The reaction was cooled to room temp, filtered through a 0.45 g frit filter and purified via preparative LC/MS to afford the product (S)-2-(tert-butoxy)-2-(2-cyano-4-(4,4-dimethylpiperidin-1-yl)-5-(4-(4-fluorophenethoxy)phenyl)-6-methylpyridin-3-yl)acetic acid (2.4 mg, 4.10 μmol, 12.62% yield). ¹H NMR (500 MHz, DMSO-d6) δ 7.36 (dd, J=8.4, 5.9 Hz, 2H), 7.27 (br d, J=7.3 Hz, 1H), 7.15-7.03 (m, 5H), 5.47 (s, 1H), 4.31-4.22 (m, 2H), 3.89 (s, 1H), 3.06 (t, J=6.6 Hz, 2H), 2.17 (s, 3H), 1.24 (br d, J=12.1 Hz, 2H), 1.18 (s, 9H), 0.80-0.50 (m, 6H). 6 protons on the piperidine ring were not resolved via ¹H NMR due to water suppression in the experiment. LCMS (M+1)=574.2.

### Example 6

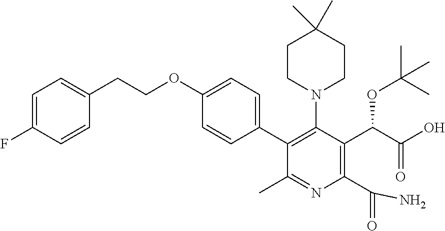 [SMILES: Cc1nc(C(N)=O)c([C@H](OC(C)(C)C)C(=O)O)c(N2CCC(C)(C)CC2)c1-c1ccc(OCCc2ccc(F)cc2)cc1]

### (S)-2-(tert-Butoxy)-2-(2-carbamoyl-4-(4, 4-dimethylpiperidin-1-yl)-5-(4-(4-fluorophenethoxy)phenyl)-6-methylpyridin-3-yl)acetic acid

To a solution of (S)-isopropyl 2-(tert-butoxy)-2-(2-cyano-4-(4,4-dimethylpiperidin-1-yl)-5-(4-(4-fluorophenethoxy)phenyl)-6-methylpyridin-3-yl)acetate (50 mg, 0.081 mmol) in EtOH (1 mL) and water (0.111 mL) was added lithium hydroxide monohydrate (34.1 mg, 0.812 mmol) and heated at 75° C. for 30 min. After 30 minutes, the LCMS indicated the reaction was complete. The reaction was cooled to room temp, filtered through a 0.45 g frit filter and purified via preparative LC/MS to afford the product (S)-2-(tert-butoxy)-2-(2-carbamoyl-4-(4,4-dimethylpiperidin-1-yl)-5-(4-(4-fluorophenethoxy)phenyl)-6-methylpyridin-3-yl)acetic acid (1.5 mg, 2.459 μmol, 3.03% yield). ¹H NMR (500 MHz, DMSO-d6) δ 7.38-7.33 (m, 2H), 7.26-7.16 (m, 1H), 7.15-7.05 (m, 5H), 5.64 (s, 1H), 4.27 (q, J=6.0 Hz, 2H), 3.06 (br t, J=6.4 Hz, 2H), 2.13 (s, 3H), 1.28-1.25 (m, 1H), 1.24-1.16 (m, 4H), 1.10 (s, 9H), 0.72 (br s, 6H). 4 protons of methylenes on the 4-fluorophenethoxy were not resolved via ¹H NMR due to water suppression in the experiment. LCMS (M+1)=592.1.

### Example 7

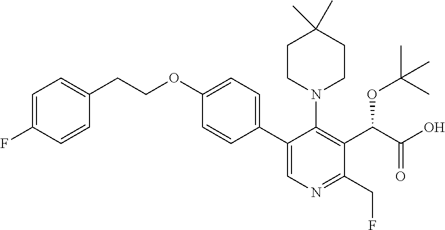 [SMILES: CC1(C)CCN(c2c(-c3ccc(OCCc4ccc(F)cc4)cc3)cnc(CF)c2[C@H](OC(C)(C)C)C(=O)O)CC1]

### (S)-2-(tert-Butoxy)-2-(4-(4, 4-dimethylpiperidin-1-yl)-2-(fluoromethyl)-5-(4-(4-fluorophenethoxy)phenyl)pyridin-3-yl)acetic acid

To an N₂ sparged solution of (S)-benzyl 2-(tert-butoxy)-2-(4-(4,4-dimethylpiperidin-1-yl)-2-(fluoromethyl)-5-(4-(4-fluorophenethoxy)phenyl)pyridin-3-yl)acetate (43 mg, 0.065 mmol) in MeOH (2 mL) was added Pd/C (6.97 mg, 6.55 μmol) and capped with a rubber septum. Hydrogen was then bubbled through the solution for 10 minutes. The reaction was then left under positive pressure of hydrogen for 1 hr. The LCMS indicated the reaction was complete. The reaction was filtered through a 0.45μ nylon frit filter and the volatiles evaporated. The crude material was taken up in MeOH and purified via preparative LC/MS to afford the product (S)-2-(tert-butoxy)-2-(4-(4,4-dimethylpiperidin-1-yl)-2-(fluoromethyl)-5-(4-(4-fluorophenethoxy)phenyl)pyridin-3-yl)acetic acid (30 mg, 0.052 mmol, 80% yield) as an off-white fluffy solid. ¹H NMR (500 MHz, methanol-d4) δ 8.18 (s, 1H), 7.36 (dd, J=8.7, 5.4 Hz, 2H), 7.27 (d, J=8.7 Hz, 2H), 7.08-7.03 (m, 4H), 5.84 (s, 1H), 5.70 (s, 1H), 5.61 (s, 1H), 4.27 (t, J=6.7 Hz, 2H), 3.12 (t, J=6.7 Hz, 2H), 1.45-1.37 (m, 3H), 1.18 (s, 9H), 0.88 (br s, 6H). 5 protons on the piperidine ring were not resolved via ¹H NMR due to water in the experiment. LCMS (M+1)=567.3.

### Example 8

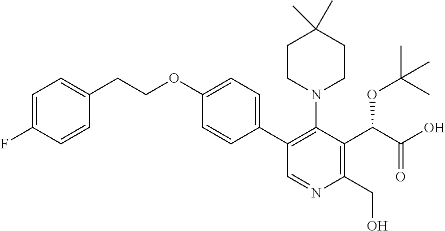 [SMILES: CC1(C)CCN(c2c(-c3ccc(OCCc4ccc(F)cc4)cc3)cnc(CO)c2[C@H](OC(C)(C)C)C(=O)O)CC1]

### (S)-2-(tert-Butoxy)-2-(4-(4, 4-dimethylpiperidin-1-yl)-5-(4-(4-fluorophenethoxy)phenyl)-2-(hydroxymethyl)pyridin-3-yl)acetic acid

A mixture of (S)-isopropyl 2-(5-bromo-4-(4,4-dimethylpiperidin-1-yl)-2-(hydroxymethyl)pyridin-3-yl)-2-(tert-butoxy)acetate (50 mg, 0.106 mmol), (4-(4-fluorophenethoxy)phenyl)boronic acid (41.4 mg, 0.159 mmol), 2-dicyclohexylphosphino-2′,6′-dimethoxybiphenyl (8.71 mg, 0.021 mmol), and potassium phosphate tribasic (169 mg, 0.795 mmol) in 1,4-dioxane (1768 μl) and water (354 μl) was bubbled with N₂ for 10 minutes. Pd(OAc)₂ (2.381 mg, 10.61 μmol) was added and the reaction was kept under positive pressure of N₂ for the duration of the reaction. The reaction was heated at 80° C. for 18 h. The reaction was cooled to RT and diluted with water and EtOAc. The organic layer was washed with brine, collected, dried over MgSO₄, filtered and volatiles evaporated to give the crude product. The crude product was purified via preparative LC/MS to afford the product (S)-2-(tert-butoxy)-2-(4-(4,4-dimethylpiperidin-1-yl)-5-(4-(4-fluorophenethoxy)phenyl)-2-(hydroxymethyl)pyridin-3-yl)acetic acid (11.3 mg, 0.018 mmol, 17.2% yield). ¹H NMR (500 MHz, methanol-d4) δ 8.20-8.00 (m, 1H), 7.40-7.35 (m, 2H), 7.30 (br s, 1H), 7.10-6.99 (m, 5H), 5.62 (br s, 1H), 4.30-4.24 (m, 2H), 3.12 (t, J=6.8 Hz, 2H), 3.05-2.87 (m, 1H), 2.84-2.70 (m, 2H), 2.68 (s, 3H), 1.49-1.39 (m, 4H), 1.20 (s, 9H), 0.93-0.89 (m, 6H). LCMS (M+1)=565.2.

### Example 9

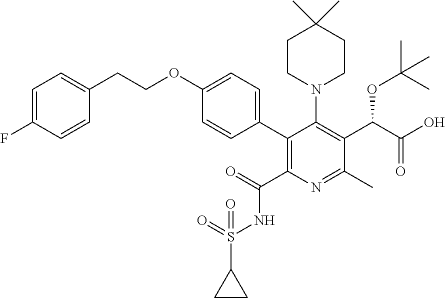 [SMILES: Cc1nc(C(=O)NS(=O)(=O)C2CC2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(6-((cyclopropylsulfonyl)carbamoyl)-4-(4, 4-dimethylpiperidin-1-yl)-5-(4-(4-fluorophenethoxy)phenyl)-2-methylpyridin-3-yl)acetic acid

To a solution of (S)-5-(1-(tert-butoxy)-2-isopropoxy-2-oxoethyl)-4-(4,4-dimethylpiperidin-1-yl)-3-(4-(4-fluorophenethoxy)phenyl)-6-methylpicolinic acid (25 mg, 0.039 mmol) in DCM (1 mL) was added DMAP (0.962 mg, 7.88 μmol) followed by cyclopropanesulfonamide (5.25 mg, 0.043 mmol) and N1-((ethylimino)methylene)-N3,N3-dimethylpropane-1,3-diamine*HCl salt (9.06 mg, 0.047 mmol). The reaction was stirred at RT for 2 hrs. The reaction volatiles were evaporated under a stream of nitrogen to afford the crude product. The crude residue was taken up in EtOH (1 mL) and water (0.1 mL) and added lithium hydroxide monohydrate (16.53 mg, 0.394 mmol). The reaction was heated at 75° C. and stirred for 18 hrs. The reaction was then cooled to RT, filtered through a 0.45 t nylon frit filter and purified via preparative LC/MS to afford the product (S)-2-(tert-butoxy)-2-(6-((cyclopropylsulfonyl)carbamoyl)-4-(4,4-dimethylpiperidin-1-yl)-5-(4-(4-fluorophenethoxy)phenyl)-2-methylpyridin-3-yl)acetic acid (13.6 mg, 0.020 mmol, 49.6% yield). ¹H NMR (500 MHz, DMSO-d6) δ 7.36 (dd, J=8.4, 5.6 Hz, 2H), 7.27 (br d, J=8.2 Hz, 1H), 7.13 (t, J=8.9 Hz, 2H), 7.04 (br d, J=7.6 Hz, 1H), 6.94 (br d, J=7.9 Hz, 1H), 6.88 (br d, J=6.1 Hz, 1H), 5.89-5.83 (m, 1H), 4.25-4.12 (m, 2H), 3.03 (brt, J=6.7 Hz, 2H), 2.94-2.84 (m, 1H), 2.44 (s, 3H), 2.41-2.35 (m, 1H), 2.16-2.06 (m, 1H), 2.03-1.92 (m, 1H), 1.56-1.45 (m, 1H), 1.34-1.19 (m, 2H), 1.14 (s, 9H), 1.08-0.97 (m, 1H), 0.88-0.59 (m, 6H), 0.55-0.32 (m, 2H). 4 protons (piperidine protons) were not resolved via ¹H NMR due to water suppression in the experiment. LCMS (M+1)=696.2.

### Example 10

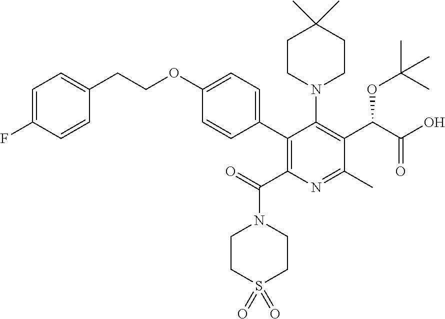 [SMILES: Cc1nc(C(=O)N2CCS(=O)(=O)CC2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-6-(1,1-dioxidothiomorpholine-4-carbonyl)-5-(4-(4-fluorophenethoxy)phenyl)-2-methylpyridin-3-yl)acetic acid

To a solution of (S)-5-(1-(tert-butoxy)-2-isopropoxy-2-oxoethyl)-4-(4,4-dimethylpiperidin-1-yl)-3-(4-(4-fluorophenethoxy)phenyl)-6-methylpicolinic acid (25 mg, 0.039 mmol), Hunig's Base (0.015 mL, 0.087 mmol), and thiomorpholine 1,1-dioxide (5.32 mg, 0.039 mmol) in DCM (1 mL) was added HATU (16.47 mg, 0.043 mmol) and stirred at RT for 1 hr. The reaction volatiles were removed via nitrogen stream to afford the crude product. The crude product was taken up in EtOH (1 mL) and water (0.1 mL) and added lithium hydroxide monohydrate (16.53 mg, 0.394 mmol) and heated to 75° C. for 18 hrs. The reaction was cooled to RT and filtered through a 0.45 g nylon frit filter and purified via preparative LC/MS to afford the product (S)-2-(tert-butoxy)-2-(4-(4,4-dimethylpiperidin-1-yl)-6-(1,1-dioxidothiomorpholine-4-carbonyl)-5-(4-(4-fluorophenethoxy)phenyl)-2-methylpyridin-3-yl)acetic acid (17.5 mg, 0.025 mmol, 62.6% yield). ¹H NMR (500 MHz, methanol-d4) δ 7.35 (dd, J=8.5, 5.5 Hz, 2H), 7.32-7.29 (m, 1H), 7.23 (dd, J=8.5, 2.1 Hz, 1H), 7.09-6.99 (m, 4H), 5.84 (s, 1H), 4.26 (t, J=6.7 Hz, 2H), 4.04-3.92 (m, 1H), 3.87-3.62 (m, 2H), 3.48-3.36 (m, 1H), 3.21-3.13 (m, 1H), 3.11 (t, J=6.7 Hz, 2H), 3.07-2.99 (m, 2H), 2.83-2.73 (m, 1H), 2.61 (s, 3H), 2.58-2.49 (m, 1H), 2.37-2.05 (m, 2H), 1.79-1.58 (m, 1H), 1.51-1.26 (m, 2H), 1.21 (s, 9H), 1.17-1.02 (m, 1H), 1.00-0.69 (m, 6H). LCMS (M+1)=710.1.

### Example 11

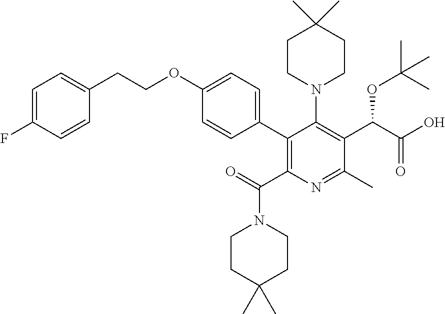 [SMILES: Cc1nc(C(=O)N2CCC(C)(C)CC2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-6-(4, 4-dimethylpiperidine-1-carbonyl)-5-(4-(4-fluorophenethoxy)phenyl)-2-methylpyridin-3-yl)acetic acid

To a solution of (S)-5-(1-(tert-butoxy)-2-isopropoxy-2-oxoethyl)-4-(4,4-dimethylpiperidin-1-yl)-3-(4-(4-fluorophenethoxy)phenyl)-6-methylpicolinic acid (25 mg, 0.039 mmol), Hunig's Base (0.015 mL, 0.087 mmol), and 4,4-dimethylpiperidine (4.46 mg, 0.039 mmol) in DCM (1 mL) was added HATU (16.47 mg, 0.043 mmol) and stirred at RT for 1 hr. The reaction was then diluted with DCM and washed with water. The organic layer was washed with brine, collected, dried over MgSO₄, filtered and volatiles evaporated to afford the crude product. The crude product was taken up in EtOH (2 mL) and water (0.2 mL) and added lithium hydroxide monohydrate (16.53 mg, 0.394 mmol) and heated to 75° C. After 18 hrs, the reaction was filtered through a 0.45p nylon frit filter and purified via preparative LC/MS to afford the product (S)-2-(tert-butoxy)-2-(4-(4,4-dimethylpiperidin-1-yl)-6-(4,4-dimethylpiperidine-1-carbonyl)-5-(4-(4-fluorophenethoxy)phenyl)-2-methylpyridin-3-yl)acetic acid (11.5 mg, 0.017 mmol, 42.4% yield). ¹H NMR (500 MHz, DMSO-d6) δ 7.36 (dd, J=8.4, 5.6 Hz, 2H), 7.20 (br d, J=7.9 Hz, 1H), 7.13 (t, J=8.9 Hz, 2H), 7.07-7.00 (m, 2H), 6.97 (dd, J=8.4, 2.3 Hz, 1H), 5.60 (s, 1H), 4.25-4.16 (m, 2H), 3.03 (br t, J=6.6 Hz, 3H), 2.99-2.76 (m, 5H), 2.44 (s, 3H), 2.18-1.95 (m, 2H), 1.62-1.48 (m, 1H), 1.37-1.20 (m, 2H), 1.10 (s, 9H), 0.98 (br d, J=7.9 Hz, 2H), 0.89-0.82 (m, 6H), 0.66 (br d, J=15.6 Hz, 6H). 4 protons of the piperidine methylene protons were not observed via ¹H NMR due to water in the experiment. LCMS (M+1)=688.3.

### Example 12

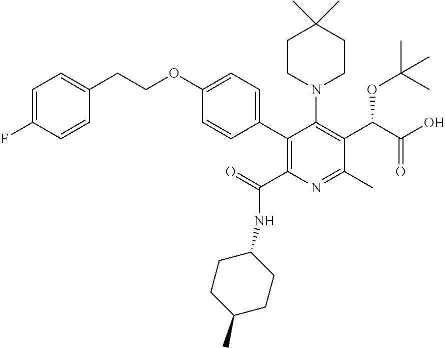 [SMILES: Cc1nc(C(=O)N[C@H]2CC[C@H](C)CC2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-(((1 r, 4S)-4-methylcyclohexyl)carbamoyl)pyridin-3-yl)acetic acid

To a solution of (S)-5-(1-(tert-butoxy)-2-isopropoxy-2-oxoethyl)-4-(4,4-dimethylpiperidin-1-yl)-3-(4-(4-fluorophenethoxy)phenyl)-6-methylpicolinic acid (25 mg, 0.039 mmol), Hunig's Base (0.015 mL, 0.087 mmol), and (1r,4r)-4-methylcyclohexanamine (4.46 mg, 0.039 mmol) in DCM (1 mL) was added HATU (16.47 mg, 0.043 mmol) and stirred at RT for 1 hr. The reaction was then diluted with DCM and washed with water. The organic layer was washed with brine, collected, dried over MgSO₄, filtered and volatiles evaporated to afford the crude product. The crude product was taken up in EtOH (2 mL) and water (0.2 mL) and added lithium hydroxide monohydrate (16.53 mg, 0.394 mmol) and heated to 75° C. for 18 hrs. The reaction was then cooled to RT and filtered through a 0.45 g nylon frit filter and purified via preparative LC/MS to afford the product (S)-2-(tert-butoxy)-2-(4-(4,4-dimethylpiperidin-1-yl)-5-(4-(4-fluorophenethoxy)phenyl)-2-methyl-6-(((1r,4S)-4-methylcyclohexyl)carbamoyl)pyridin-3-yl)acetic acid (8.4 mg, 0.012 mmol, 31.0% yield). ¹H NMR (500 MHz, DMSO-d6) δ 7.88 (d, J=8.2 Hz, 1H), 7.35 (dd, J=8.4, 5.6 Hz, 2H), 7.18-7.09 (m, 4H), 7.00-6.88 (m, 2H), 5.79 (br s, 1H), 4.28-4.15 (m, 2H), 3.02 (t, J=6.7 Hz, 2H), 2.91-2.82 (m, 1H), 2.47 (s, 3H), 2.18-2.11 (m, 1H), 2.00 (br t, J=11.4 Hz, 1H), 1.59-1.39 (m, 4H), 1.37-1.19 (m, 3H), 1.13 (s, 9H), 1.08-0.97 (m, 2H), 0.88-0.71 (m, 8H), 0.63 (s, 3H). 4 protons of the piperidine methylene protons were not observed via ¹H NMR due to water in the experiment. LCMS (M+1)=688.3.

### Example 13

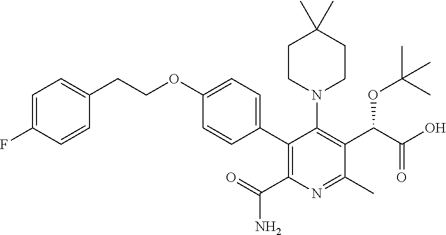 [SMILES: Cc1nc(C(N)=O)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(6-carbamoyl-4-(4, 4-dimethylpiperidin-1-yl)-5-(4-(4-fluorophenethoxy)phenyl)-2-methylpyridin-3-yl)acetic acid

To a solution of (S)-5-(1-(tert-butoxy)-2-isopropoxy-2-oxoethyl)-4-(4,4-dimethylpiperidin-1-yl)-3-(4-(4-fluorophenethoxy)phenyl)-6-methylpicolinic acid (25 mg, 0.039 mmol), Hunig's Base (0.015 mL, 0.087 mmol), and ammonium chloride (10.53 mg, 0.197 mmol) in DCM (1 mL) was added HATU (16.47 mg, 0.043 mmol) and stirred at RT for 1 hr. The reaction was then diluted with DCM and washed with water. The organic layer was washed with brine, collected, dried over MgSO₄, filtered and volatiles evaporated to afford the crude product. The crude product was taken up in EtOH (1 mL) and water (0.1 mL) and added lithium hydroxide monohydrate (16.53 mg, 0.394 mmol) and heated to 75° C. for 18 hrs. The reaction was then cooled to RT and filtered through a 0.45 g nylon frit filter and purified via preparative LC/MS to afford the product (S)-2-(tert-butoxy)-2-(6-carbamoyl-4-(4,4-dimethylpiperidin-1-yl)-5-(4-(4-fluorophenethoxy)phenyl)-2-methylpyridin-3-yl)acetic acid (9.7 mg, 0.016 mmol, 41.2% yield). ¹H NMR (500 MHz, DMSO-d6) δ 7.55 (s, 1H), 7.37 (dd, J=8.5, 5.8 Hz, 2H), 7.20 (br d, J=8.5 Hz, 1H), 7.14 (t, J=8.9 Hz, 2H), 7.08 (br d, J=10.1 Hz, 1H), 7.01 (s, 1H), 6.99-6.95 (m, 1H), 6.91 (dd, J=8.4, 2.6 Hz, 1H), 5.66 (s, 1H), 4.25-4.16 (m, 2H), 3.06-3.03 (m, 2H), 2.85-2.78 (m, 1H), 2.47 (s, 3H), 2.16-2.09 (m, 1H), 1.57-1.47 (m, 1H), 1.35-1.14 (m, 2H), 1.11 (s, 9H), 1.05-0.98 (m, 1H), 0.85 (s, 3H), 0.63 (s, 3H). 3 protons of the piperidine methylene protons were not observed via ¹H NMR due to water in the experiment. LCMS (M+1)=592.2.

### Example 14

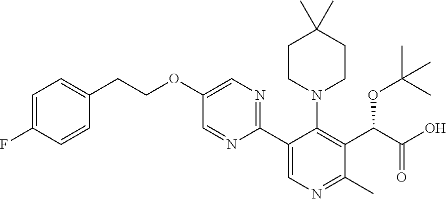 [SMILES: Cc1ncc(-c2ncc(OCCc3ccc(F)cc3)cn2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(5-(4-fluorophenethoxy)pyrimidin-2-yl)-2-methylpyridin-3-yl)acetic acid

To a solution of (S)-isopropyl 2-(tert-butoxy)-2-(4-(4,4-dimethylpiperidin-1-yl)-5-(5-(4-fluorophenethoxy)pyrimidin-2-yl)-2-methylpyridin-3-yl)acetate (20 mg, 0.034 mmol) in EtOH (1 mL) and water (0.111 mL) was added lithium hydroxide monohydrate (14.16 mg, 0.337 mmol) and heated at 75° C. for 24 hrs. The reaction was cooled to RT, filtered through a nylon 0.45 t frit filter and purified via preparative LC/MS to afford the product (S)-2-(tert-butoxy)-2-(4-(4,4-dimethylpiperidin-1-yl)-5-(5-(4-fluorophenethoxy)pyrimidin-2-yl)-2-methylpyridin-3-yl)acetic acid (8.4 mg, 0.015 mmol, 45.2% yield). ¹H NMR (500 MHz, methanol-d4) δ 8.61 (s, 2H), 8.29 (s, 1H), 7.42-7.29 (m, 2H), 7.04 (t, J=8.8 Hz, 2H), 5.96 (s, 1H), 4.50-4.42 (m, 2H), 3.16 (t, J=6.6 Hz, 2H), 2.99-2.74 (m, 4H), 2.64 (s, 3H), 1.44 (br d, J=4.4 Hz, 4H), 1.21 (s, 9H), 0.92 (s, 6H) LCMS (M+1)=551.3.

### Example 15

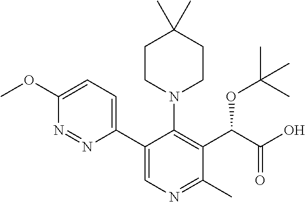 [SMILES: COc1ccc(-c2cnc(C)c([C@H](OC(C)(C)C)C(=O)O)c2N2CCC(C)(C)CC2)nn1]

### (S)-2-(tert-Butoxy)-2-(4-(4, 4-dimethylpiperidin-1-yl)-5-(6-methoxypyridazin-3-yl)-2-methylpyridin-3-yl)acetic acid

To a solution of (S)-isopropyl 2-(tert-butoxy)-2-(4-(4,4-dimethylpiperidin-1-yl)-5-(6-methoxypyridazin-3-yl)-2-methylpyridin-3-yl)acetate (22 mg, 0.045 mmol) in EtOH (1 mL) and water (0.111 mL) was added lithium hydroxide monohydrate (19.05 mg, 0.454 mmol) and heated at 75° C. for 24 hrs. The reaction was cooled to RT, filtered through a nylon 0.45μ frit filter and purified via preparative LC/MS to afford the product (S)-2-(tert-butoxy)-2-(4-(4,4-dimethylpiperidin-1-yl)-5-(6-methoxypyridazin-3-yl)-2-methylpyridin-3-yl)acetic acid (16.1 mg, 0.036 mmol, 80% yield). ¹H NMR (500 MHz, methanol-d₄) δ 8.20 (s, 1H), 7.70 (d, J=9.2 Hz, 1H), 7.31 (d, J=8.8 Hz, 1H), 5.96 (s, 1H), 4.18 (s, 3H), 3.03-2.68 (m, 4H), 2.66 (s, 3H), 1.50-1.39 (m, 4H), 1.25-1.20 (m, 9H), 0.90 (s, 6H). LCMS (M+1)=443.2.

### Example 16

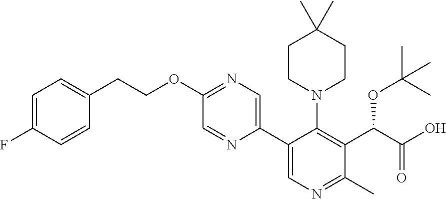 [SMILES: Cc1ncc(-c2cnc(OCCc3ccc(F)cc3)cn2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(5-(4-fluorophenethoxy)pyrazin-2-yl)-2-methylpyridin-3-yl)acetic acid

To a solution of (S)-isopropyl 2-(tert-butoxy)-2-(4-(4,4-dimethylpiperidin-1-yl)-5-(5-(4-fluorophenethoxy)pyrazin-2-yl)-2-methylpyridin-3-yl)acetate (30 mg, 0.051 mmol) in EtOH (1 mL) and water (0.111 mL) was added lithium hydroxide monohydrate (21.24 mg, 0.506 mmol) and heated at 75° C. for 24 hrs. The reaction was cooled to RT, filtered through a nylon 0.45 g frit filter and purified via preparative LC/MS to afford the product (S)-2-(tert-butoxy)-2-(4-(4,4-dimethylpiperidin-1-yl)-5-(5-(4-fluorophenethoxy)pyrazin-2-yl)-2-methylpyridin-3-yl)acetic acid (7.5 mg, 0.014 mmol, 26.9% yield). ¹H NMR (500 MHz, methanol-d₄) δ 8.29 (d, J=1.5 Hz, 1H), 8.24 (d, J=1.1 Hz, 1H), 8.14 (s, 1H), 7.35-7.30 (m, 2H), 7.05-7.00 (m, 2H), 5.88 (s, 1H), 4.66 (t, J=6.8 Hz, 2H), 3.14 (t, J=6.8 Hz, 2H), 3.07-2.89 (m, 2H), 2.83-2.74 (m, 2H), 2.65 (s, 3H), 1.43 (br s, 4H), 1.20 (s, 9H), 0.90 (s, 6H). LCMS (M+1)=551.3.

### Example 17

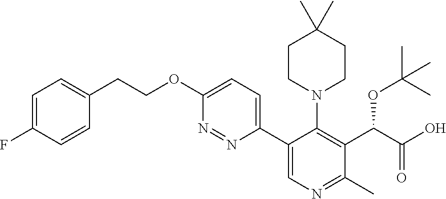 [SMILES: Cc1ncc(-c2ccc(OCCc3ccc(F)cc3)nn2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(6-(4-fluorophenethoxy)pyridazin-3-yl)-2-methylpyridin-3-yl)acetic acid

To a solution of (S)-isopropyl 2-(tert-butoxy)-2-(4-(4,4-dimethylpiperidin-1-yl)-5-(6-(4-fluorophenethoxy)pyridazin-3-yl)-2-methylpyridin-3-yl)acetate (47 mg, 0.079 mmol) in EtOH (1 mL) and water (0.111 mL) was added lithium hydroxide monohydrate (33.3 mg, 0.793 mmol) and heated at 75° C. for 24 hrs. The reaction was cooled to RT, filtered through a nylon 0.45 g frit filter and purified via preparative LC/MS to afford the product (S)-2-(tert-butoxy)-2-(4-(4,4-dimethylpiperidin-1-yl)-5-(6-(4-fluorophenethoxy)pyridazin-3-yl)-2-methylpyridin-3-yl)acetic acid (25.6 mg, 0.046 mmol, 58.0% yield). 1H NMR (500 MHz, methanol-d4) δ 8.08 (s, 1H), 7.55 (d, J=9.5 Hz, 1H), 7.23 (dd, J=8.6, 5.4 Hz, 2H), 7.09 (d, J=9.5 Hz, 1H), 6.98 (t, J=8.8 Hz, 2H), 5.80 (s, 1H), 4.66 (dt, J=13.0, 7.2 Hz, 1H), 4.37 (dt, J=13.0, 7.2 Hz, 1H), 3.18 (t, J=7.2 Hz, 2H), 2.89-2.70 (m, 2H), 2.63 (s, 3H), 1.56-1.40 (m, 4H), 1.20 (s, 9H), 0.97 (s, 6H). LCMS (M+1)=551.2.

### Example 18

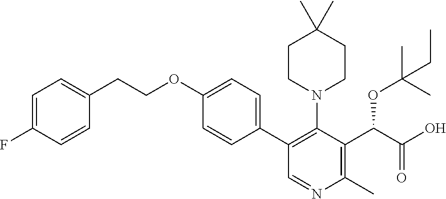 [SMILES: CCC(C)(C)O[C@H](C(=O)O)c1c(C)ncc(-c2ccc(OCCc3ccc(F)cc3)cc2)c1N1CCC(C)(C)CC1]

### (2S)-2-[4-(4, 4-Dimethylpiperidin-1-yl)-5-{4-[2-(4-fluorophenyl)ethoxy]phenyl}-2-methylpyridin-3-yl]-2-[(2-methylbutan-2-yl)oxy]acetic acid

(S)-Isopropyl 2-(5-bromo-4-(4,4-dimethylpiperidin-1-yl)-2-methylpyridin-3-yl)-2-(tert-pentyloxy)acetate (45 mg, 0.096 mmol), (4-(4-fluorophenethoxy)phenyl)boronic acid (37 mg, 0.14 mmol), (2-dicyclohexylphosphino-2′,6′-dimethoxybiphenyl) [2-(2′-amino-1,1′-biphenyl)]palladium(II) methanesulfonate (7 mg, 10 μmol), potassium phosphate tribasic (61 mg, 0.29 mmol) were combined under N₂ (g). 1,4-Dioxane (1.6 ml) and water (0.32 ml) was added under N₂ (g). The reaction was stirred at 80° C. for 1 hr. The reaction was concentrated, adsorbed onto celite and was purified on silica gel (Biotage, EtOAc/hexanes gradient, 0-100% over 10 CVs). The major product was isolated and taken up in EtOH and 0.1 mL of 5 N NaOH was added and the reaction was stirred at 100 C for 5 hrs. Then the crude material was purified via preparative LC/MS to afford (2S)-2-[4-(4,4-dimethylpiperidin-1-yl)-5-{4-[2-(4-fluorophenyl)ethoxy]phenyl}-2-methylpyridin-3-yl]-2-[(2-methylbutan-2-yl)oxy]acetic acid (15.3 mg). LCMS (M+H)=563.24. ¹H NMR (500 MHz, DMSO-d₆) δ 8.02 (s, 1H), 7.36 (t, J=6.6 Hz, 2H), 7.20 (d, J=8.8 Hz, 2H), 7.14-7.09 (m, 2H), 7.02 (d, J=7.5 Hz, 2H), 5.83 (s, 1H), 4.28-4.20 (m, 2H), 3.05 (t, J=6.8 Hz, 1H), 1.51-1.35 (m, 3H), 1.30 (br s, 4H), 1.11 (s, 3H), 1.03 (s, 3H), 0.80 (br s, 7H), 0.71 (t, J=7.3 Hz, 4H). Not all of the piperidine protons were well resolved.

### Example 19

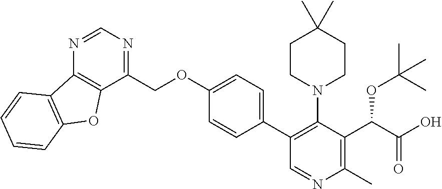 [SMILES: Cc1ncc(-c2ccc(OCc3ncnc4c3oc3ccccc34)cc2)c(N2CCC(C)(C)CC2)c1[C@H](OC(C)(C)C)C(=O)O]

### (2S)-2-(tert-Butoxy)-2-[4-(4, 4-dimethylpiperidin-1-yl)-2-methyl-5-[4-({8-oxa-3, 5-diazatricyclo[7.4.0.0^{2,7}]trideca-1(9), 2(7), 3, 5,10,12-hexaen-6-yl}methoxy)phenyl]pyridin-3-yl]acetic acid

4-(Chloromethyl)benzofuro[3,2-d]pyrimidine (12 mg, 0.056 mmol) was added to a stirring solution of (S)-2-(tert-butoxy)-2-(4-(4,4-dimethylpiperidin-1-yl)-5-(4-hydroxyphenyl)-2-methylpyridin-3-yl)acetic acid (20 mg, 0.047 mmol) and K₂CO₃ (32 mg, 0.23 mmol) and KI (8 mg, 0.05 mmol) in ACN (1 ml) at rt. The reaction was stirred at 80° C. for 12 hrs and then the crude material was purified via preparative LC/MS to afford (2S)-2-(tert-butoxy)-2-[4-(4,4-dimethylpiperidin-1-yl)-2-methyl-5-[4-({8-oxa-3,5-diazatricyclo[7.4.0.0^{2,7}]trideca-1(9),2(7),3,5,10,12-hexaen-6-yl}methoxy)phenyl]pyridin-3-yl]acetic acid (10.0 mg). LCMS (M+H)=609.28. ¹H NMR (500 MHz, DMSO-d6) δ 9.14 (s, 1H), 8.28 (d, J=7.7 Hz, 1H), 8.01 (s, 1H), 7.88-7.79 (m, 3H), 7.61 (t, J=7.3 Hz, 1H), 7.02 (d, J=8.4 Hz, 3H), 6.80 (d, J=8.4 Hz, 3H), 6.13 (br s, 1H), 5.76 (d, J=13.9 Hz, 1H), 5.56 (d, J=13.9 Hz, 1H), 3.17 (s, 1H), 2.54-2.51 (m, 4H), 1.17 (s, 15H), 0.67 (br s, 8H). Not all of the piperidine protons were well resolved.

### Example 20

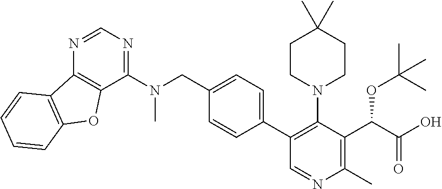 [SMILES: Cc1ncc(-c2ccc(CN(C)c3ncnc4c3oc3ccccc34)cc2)c(N2CCC(C)(C)CC2)c1[C@H](OC(C)(C)C)C(=O)O]

### (2S)-2-(tert-Butoxy)-2-[4-(4, 4-dimethylpiperidin-1-yl)-2-methyl-5-(4-{[methyl({8-oxa-3, 5-diazatricyclo[7.4.0.0^{2,7}]trideca-1(9),2(7),3,5,10,12-hexaen-6-yl})amino]methyl}phenyl)pyridin-3-yl]acetic acid

(S)-Isopropyl 2-(5-bromo-4-(4,4-dimethylpiperidin-1-yl)-2-methylpyridin-3-yl)-2-(tert-butoxy)acetate (25 mg, 0.055 mmol), N-methyl-N-(4-(4,4,5,5-tetramethyl-1,3,2-dioxaborolan-2-yl)benzyl)benzofuro[3,2-d]pyrimidin-4-amine (27 mg, 0.066 mmol), chloro(2-dicyclohexylphosphino-2′,6′-dimethoxy-1,1′-biphenyl) [2-(2′-amino-1,1′-biphenyl)]palladium(II) (3.95 mg, 5.49 μmol), potassium phosphate tribasic (35 mg, 0.17 mmol) were combined under N₂ (g). 1,4-Dioxane (1 ml) and water (0.2 ml) was added under N₂ (g). The reaction was stirred at 80° C. for 1 hr. The reaction was concentrated and subjected to hydrolysis conditions (0.1 mL 5 N NaOH in 1.5 mL of EtOH) stirring at 100° C. for 5 hrs. The sample was then the crude material was purified via preparative LC/MS to afford (2S)-2-(tert-butoxy)-2-[4-(4,4-dimethylpiperidin-1-yl)-2-methyl-5-(4-{[methyl({8-oxa-3,5-diazatricyclo[7.4.0.0^{2,7}]trideca-1 (9),2(7),3,5,10,12-hexaen-6-yl})amino]methyl}phenyl)pyridin-3-yl]acetic acid (12.0 mg) LCMS (M+H)=622.13. ¹H NMR (500 MHz, DMSO-d6) δ 8.54 (s, 1H), 8.11 (d, J=8.1 Hz, 1H), 8.03 (s, 1H), 7.78-7.74 (m, 1H), 7.68 (t, J=7.7 Hz, 1H), 7.50 (t, J=7.5 Hz, 1H), 7.41 (br d, J=7.3 Hz, 2H), 7.29 (br d, J=7.7 Hz, 2H), 5.78 (s, 1H), 5.21 (br s, 2H), 2.55-2.52 (m, 1H), 2.48-2.44 (m, 3H), 1.91 (s, 1H), 1.21 (br s, 3H), 1.09 (s, 11H), 0.80 (br s, 4H), 0.48 (br s, 3H). Not all of the piperidine protons were well resolved.

### Example 21

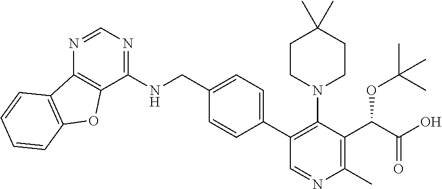 [SMILES: Cc1ncc(-c2ccc(CNc3ncnc4c3oc3ccccc34)cc2)c(N2CCC(C)(C)CC2)c1[C@H](OC(C)(C)C)C(=O)O]

### (2S)-2-(tert-Butoxy)-2-[4-(4, 4-dimethylpiperidin-1-yl)-2-methyl-5-{4-[({8-oxa-3, 5-diazatricyclo[7.4.0.0^{2,7}]trideca-1(9), 2(7), 3,5,10, 12-hexaen-6-yl}amino)methyl]phenyl}pyridin-3-yl]acetic acid

(S)-Isopropyl 2-(5-bromo-4-(4,4-dimethylpiperidin-1-yl)-2-methylpyridin-3-yl)-2-(tert-butoxy)acetate (25 mg, 0.055 mmol), N-(4-(4,4,5,5-tetramethyl-1,3,2-dioxaborolan-2-yl)benzyl)benzofuro[3,2-d]pyrimidin-4-amine (26 mg, 0.066 mmol), chloro(2-dicyclohexylphosphino-2′,6′-dimethoxy-1,1′-biphenyl) [2-(2′-amino-1,1′-biphenyl)]palladium(II) (4 mg, 5 μmol), potassium phosphate tribasic (35 mg, 0.17 mmol) were combined under N₂ (g). 1,4-Dioxane (1 ml) and water (0.2 ml) was added under N₂ (g). The reaction was stirred at 80° C. for 1 hr. The reaction was concentrated taken up in 1.5 mL of EtOH and treated with 0.1 mL of 5 N NaOH and stirred at 100° C. for 5 hrs. Then the crude material was purified via preparative LC/MS to afford (2S)-2-(tert-butoxy)-2-[4-(4,4-dimethylpiperidin-1-yl)-2-methyl-5-{4-[({8-oxa-3,5-diazatricyclo[7.4.0.0^{2,7}]trideca-1(9),2(7),3,5,10,12-hexaen-6-yl}amino)methyl]phenyl}pyridin-3-yl]acetic acid (11.3 mg). LCMS (M+H)=608.25. ¹H NMR (500 MHz, DMSO-d6) δ 8.78 (br s, 1H), 8.48 (s, 1H), 8.10 (d, J=8.1 Hz, 1H), 8.02 (s, 1H), 7.82 (d, J=8.4 Hz, 1H), 7.71 (t, J=7.7 Hz, 1H), 7.53-7.47 (m, 3H), 7.27 (d, J=7.7 Hz, 2H), 5.81 (s, 1H), 4.84 (br d, J=5.1 Hz, 2H), 2.55 (s, 1H), 2.49-2.45 (m, 3H), 1.26 (br s, 2H), 1.11 (s, 10H), 0.84 (br s, 4H), 0.60 (br s, 3H). Not all of the piperidine protons were well resolved.

### Example 22

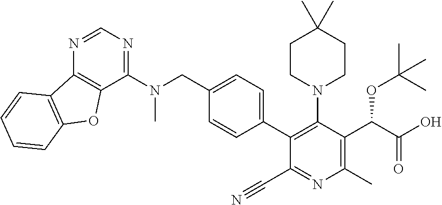 [SMILES: Cc1nc(C#N)c(-c2ccc(CN(C)c3ncnc4c3oc3ccccc34)cc2)c(N2CCC(C)(C)CC2)c1[C@H](OC(C)(C)C)C(=O)O]

### (2S)-2-(tert-Butoxy)-2-[6-cyano-4-(4, 4-dimethylpiperidin-1-yl)-2-methyl-5-(4-{[methyl({8-oxa-3, 5-diazatricyclo[7.4.0.0^{2,7}]trideca-1 (9), 2(7), 3,5,10, 12-hexaen-6-yl})amino]methyl}phenyl)pyridin-3-yl]acetic acid

(S)-Isopropyl 2-(5-bromo-6-cyano-4-(4,4-dimethylpiperidin-1-yl)-2-methylpyridin-3-yl)-2-(tert-butoxy)acetate (30 mg, 0.062 mmol), N-methyl-N-(4-(4,4,5,5-tetramethyl-1,3,2-dioxaborolan-2-yl)benzyl)benzofuro[3,2-d]pyrimidin-4-amine (29 mg, 0.069 mmol), chloro(2-dicyclohexylphosphino-2′,6′-dimethoxy-1,1′-biphenyl) [2-(2′-amino-1,1′-biphenyl)]palladium(II) (4 mg, 5 μmol) and potassium phosphate tribasic (40 mg, 0.19 mmol) were combined under N₂. 1,4-Dioxane (1 ml) and water (0.2 ml) was added under N₂. The reaction was heated at 80° C. for 1 h. The reaction was concentrated, adsorbed onto celite and was purified on silica gel (Biotage, EtOAc/hexanes gradient, 0-100% over 10 CVs) isolated (S)-isopropyl 2-(5-(4-((benzofuro[3,2-d]pyrimidin-4-yl(methyl)amino)methyl)phenyl)-6-cyano-4-(4,4-dimethylpiperidin-1-yl)-2-methylpyridin-3-yl)-2-(tert-butoxy)acetate consistent by LCMS. This material was taken up in 3 mL of EtOH and it was added lithium hydroxide hydrate (5 mg, 0.1 mmol) dissolved in 0.3 mL of H₂O. The reaction was stirred at 70° C. overnight. Additional lithium hydroxide hydrate (5 mg, 0.1 mmol) dissolved in 0.3 mL of H₂O. was added and the reaction was stirred at 80° C. for 6 hrs. LCMS showed a mixture of products, one being the expected product. The crude material was purified via preparative LC/MS to afford desired compound (6.2 mg). LCMS (M+H)=647.28. ¹H NMR (500 MHz, DMSO-d₆) δ 8.55 (s, 1H), 8.11 (d, J=7.7 Hz, 1H), 7.76 (d, J=8.4 Hz, 1H), 7.69 (t, J=7.7 Hz, 1H), 7.57-7.43 (m, 5H), 7.26 (br d, J=7.3 Hz, 1H), 5.70 (s, 1H), 5.29-5.21 (m, 3H), 3.45 (s, 1H), 2.55-2.53 (m, 4H), 1.92 (s, 1H), 1.17 (br s, 3H), 1.11 (s, 12H), 0.58 (br s, 6H).

### Example 23

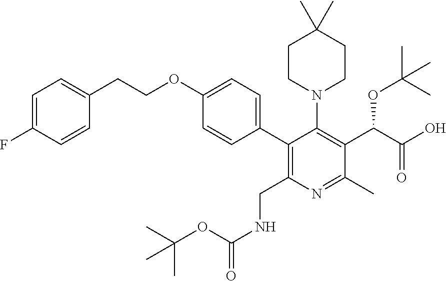 [SMILES: Cc1nc(CNC(=O)OC(C)(C)C)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(6-(((tert-butoxycarbonyl)amino)methyl)-4-(4, 4-dimethylpiperidin-1-yl)-5-(4-(4-fluorophenethoxy)phenyl)-2-methylpyridin-3-yl)acetic acid

NaH (60% dispersion on oil) (2.6 mg, 0.064 mmol) was added to a stirring solution of (S)-isopropyl 2-(6-(bromomethyl)-4-(4,4-dimethylpiperidin-1-yl)-5-(4-(4-fluorophenethoxy)phenyl)-2-methylpyridin-3-yl)-2-(tert-butoxy)acetate (22 mg, 0.032 mmol) and tert-butyl carbamate (5.7 mg, 0.048 mmol) in DMF (1 mL) at rt. The mixture was allowed to stir at this temp for 4 h. LCMS shows quant conversion to the desired product. The mixture was concentrated to a brown oil. This oil was then taken up in EtOH (1 mL) and 5M NaOH (0.097 mL, 0.483 mmol) was added. The mixture was heated to 80° C. and stirred at this temp for 2 h. The mixture was filtered and then purified via preparative LC/MS to give (S)-2-(tert-butoxy)-2-(6-(((tert-butoxycarbonyl)amino)methyl)-4-(4,4-dimethylpiperidin-1-yl)-5-(4-(4-fluorophenethoxy)phenyl)-2-methylpyridin-3-yl)acetic acid (4.8 mg, 22% yield). LCMS (M+H)=678.31.

### Example 24

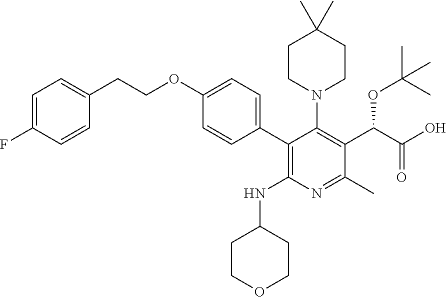 [SMILES: Cc1nc(NC2CCOCC2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-((tetrahydro-2H-pyran-4-yl)amino)pyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol) and tetrahydro-2H-pyran-4-amine (0.011 mL, 0.108 mmol) in NMP (0.63 mL) was heated at 180° C. for 3 h. Ethanol (0.5 mL) and 5 M NaOH (0.054 mL, 0.271 mmol) were added and the mixture was heated at 80° C. for 7 h, cooled to ambient temperature, and filtered. The crude mixture was purified via preparative HPLC to afford desired product (9.6 mg, 55%). LCMS (M+1)=648.35.

### Example 25

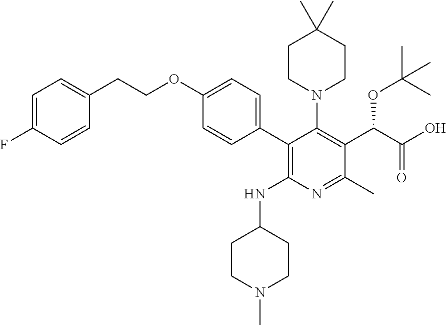 [SMILES: Cc1nc(NC2CCN(C)CC2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-((1-methylpiperidin-4-yl)amino)pyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.06 g, 0.081 mmol) and 1-methylpiperidin-4-amine (0.041 mL, 0.325 mmol) in NMP (2 mL) was heated at 180° C. for 3 h and cooled to ambient temperature. The reaction mixture was taken up in water and the resultant suspension was filtered. The filter cake was washed with water, suction dried. The solid was taken up in ethyl acetate, dried over (Na₂SO₄), and concentrated. The residue was taken up in ethanol (1 mL) and 5 M NaOH (0.162 mL, 0.812 mmol) was added. The mixture was heated at 80° C. for 3 h, cooled to ambient temperature, and filtered. The crude mixture was purified via preparative HPLC to afford the desired product (15.9 mg, 30%). ¹H NMR (500 MHz, DMSO-d₆) δ 7.37 (dd, J=8.4, 5.5 Hz, 2H), 7.23-7.01 (m, 6H), 5.71 (br. s., 1H), 4.33-4.18 (m, 2H), 3.96 (d, J=7.7 Hz, 1H), 3.77 (br. s., 1H), 3.05 (t, J=6.6 Hz, 2H), 2.74 (t, J=11.2 Hz, 1H), 2.32 (s, 3H), 2.24-1.98 (m, 5H), 1.96-1.70 (m, 3H), 1.48 (d, J=14.3 Hz, 1H), 1.37-1.09 (m, 16H), 1.00 (d, J=11.0 Hz, 1H), 0.89-0.81 (m, 3H), 0.58 (s, 3H). LCMS (M+1)=661.4.

### Example 26

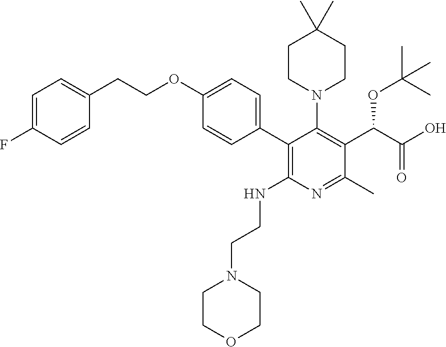 [SMILES: Cc1nc(NCCN2CCOCC2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-((2-morpholinoethyl)amino)pyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol) and 2-morpholinoethanamine (0.014 g, 0.108 mmol) in NMP (0.63 mL) was heated at 180° C. for 3 h. Ethanol (0.5 mL) and 5 M NaOH (0.054 mL, 0.271 mmol) were added and the mixture was heated at 80° C. for 7 h, cooled to ambient temperature, and filtered. The crude mixture was purified via preparative HPLC to afford the desired product (12.3 mg, 67%). ¹H NMR (500 MHz, DMSO-d₆) δ 7.37 (dd, J=8.4, 5.9 Hz, 2H), 7.24-7.00 (m, 6H), 5.72 (br. s., 1H), 4.82 (t, J=4.6 Hz, 1H), 4.31-4.17 (m, 2H), 3.91-3.79 (m, 1H), 3.32-3.17 (m, 1H), 3.06 (t, J=6.6 Hz, 2H), 2.84-2.71 (m, 1H), 2.42-2.11 (m, 10H), 2.08-1.96 (m, 1H), 1.54-1.39 (m, 1H), 1.33-1.22 (m, 4H), 1.21-1.09 (m, 10H), 1.01 (d, J=11.7 Hz, 1H), 0.90-0.80 (m, 3H), 0.59 (s, 3H). LCMS (M+1)=677.3.

### Example 27

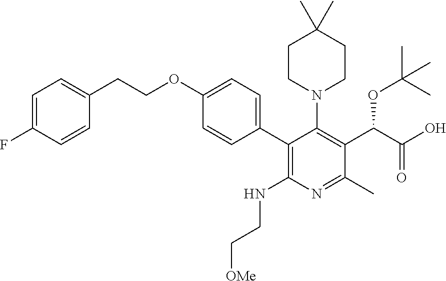 [SMILES: COCCNc1nc(C)c([C@H](OC(C)(C)C)C(=O)O)c(N2CCC(C)(C)CC2)c1-c1ccc(OCCc2ccc(F)cc2)cc1]

### (S)-2-(tert-Butoxy)-2-(4-(4, 4-dimethylpiperidin-1-yl)-5-(4-(4-fluorophenethoxy)phenyl)-6-((2-methoxyethyl)amino)-2-methylpyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol) and 2-methoxyethanamine (8.13 mg, 0.108 mmol) in NMP (0.63 mL) was heated at 180° C. for 3 h (LCMS showed the desired product peak as the major peak). Ethanol (0.5 mL) and 5 M NaOH (0.054 mL, 0.271 mmol) were added and the mixture was heated at 80° C. for 4.5 h, cooled to ambient temperature, and filtered. The crude mixture was purified via preparative HPLC to afford the desired product (8.4 mg, 50%). LCMS (M+1)=622.3.

### Example 28

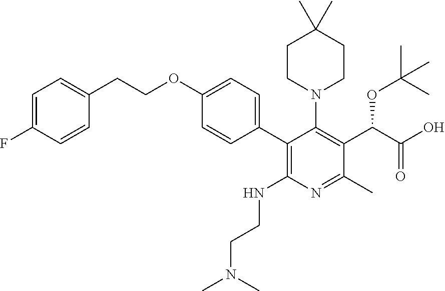 [SMILES: Cc1nc(NCCN(C)C)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(6-((2-(dimethylamino)ethyl)amino)-4-(4, 4-dimethylpiperidin-1-yl)-5-(4-(4-fluorophenethoxy)phenyl)-2-methylpyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol) and N1,N1-dimethylethane-1,2-diamine (9.54 mg, 0.108 mmol) in NMP (0.63 mL) was heated at 180° C. for 3 h. Ethanol (0.5 mL) and 5 M NaOH (0.054 mL, 0.271 mmol) were added and the mixture was heated at 80° C. for 4.5 h, cooled to ambient temperature, and filtered. The crude mixture was purified via preparative HPLC to afford the desired product (7.3 mg, 43%). ¹H NMR (500 MHz, DMSO-d₆) δ 7.37 (dd, J=8.4, 5.5 Hz, 2H), 7.20-6.97 (m, 6H), 5.71 (br. s., 1H), 4.65 (t, J=5.3 Hz, 1H), 4.31-4.15 (m, 2H), 3.26 (br. s., 1H), 3.05 (t, J=6.6 Hz, 2H), 2.75 (t, J=11.9 Hz, 1H), 2.55 (s, 5H), 2.35-2.24 (m, 5H), 2.16 (d, J=11.0 Hz, 1H), 2.07 (s, 6H), 1.47 (br. s., 1H), 1.32-1.26 (m, 1H), 1.13 (s, 9H), 1.00 (d, J=12.5 Hz, 1H), 0.89-0.81 (m, 3H), 0.59 (s, 3H). LCMS (M+1)=635.3.

### Example 29

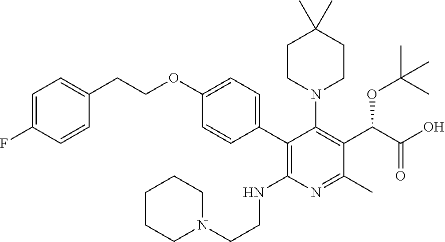 [SMILES: Cc1nc(NCCN2CCCCC2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-((2-(piperidin-1-yl)ethyl)amino)pyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol) and 2-(piperidin-1-yl)ethanamine (0.014 g, 0.108 mmol) in NMP (0.63 mL) was heated at 180° C. for 3 h. Ethanol (0.5 mL) and 5 M NaOH (0.054 mL, 0.27 mmol). The crude mixture was purified via preparative HPLC to afford the desired product (7.6 mg, 40%). LCMS (M+1)=675.4.

### Example 30

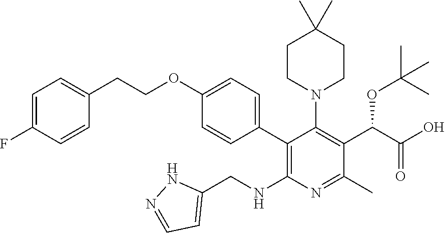 [SMILES: Cc1nc(NCc2ccn[nH]2)c(-c2ccc(OCCc3ccc(F)cc3)cc2)c(N2CCC(C)(C)CC2)c1[C@H](OC(C)(C)C)C(=O)O]

### (S)-2-(6-(((1H-Pyrazol-5-yl)methyl)amino)-4-(4, 4-dimethylpiperidin-1-yl)-5-(4-(4-fluorophenethoxy)phenyl)-2-methylpyridin-3-yl)-2-(tert-butoxy)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol) and (1H-pyrazol-5-yl)methanamine (10.52 mg, 0.108 mmol) in NMP (0.63 mL) was heated at 180° C. for 3 h. Ethanol (0.5 mL) and 5 M NaOH (0.054 mL, 0.271 mmol) were added and the mixture was heated at 80° C. for 4.5 h, cooled to ambient temperature, and filtered. The crude mixture was purified via preparative HPLC to afford the desired product (2.3 mg, 13%). LCMS (M+1)=675.4.

### Example 31

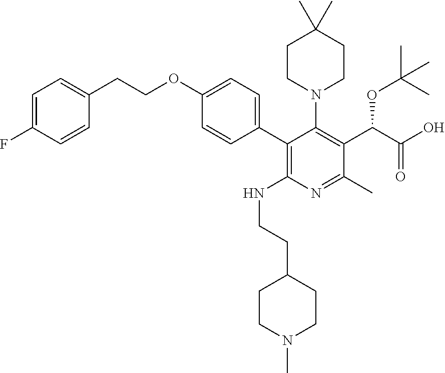 [SMILES: Cc1nc(NCCC2CCN(C)CC2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-((2-(1-methylpiperidin-4-yl)ethyl)amino)pyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol) and 2-(1-methylpiperidin-4-yl)ethanamine (0.015 g, 0.108 mmol) in NMP (0.63 mL) was heated at 180° C. for 2 h. Ethanol (0.5 mL) and 5 M NaOH (0.054 mL, 0.271 mmol) were added and the mixture was heated at 80° C. for 4.5 h, cooled to ambient temperature, and filtered. The crude mixture was purified via preparative HPLC to afford the desired product (3.1 mg, 16%). ¹H NMR (500 MHz, DMSO-d₆) δ 7.37 (dd, J=8.3, 5.7 Hz, 2H), 7.22-6.99 (m, 6H), 5.62 (s, 1H), 4.30-4.14 (m, 2H), 3.25-3.14 (m, 1H), 3.05 (t, J=6.6 Hz, 2H), 2.77-2.64 (m, 3H), 2.31 (s, 3H), 2.21-2.08 (m, 4H), 1.94-1.82 (m, 2H), 1.76 (br. s., 2H), 1.59-1.41 (m, 3H), 1.28 (br. s., 3H), 1.20-0.92 (m, 14H), 0.82 (s, 3H), 0.58 (s, 3H). LCMS (M+1)=689.3.

### Example 32

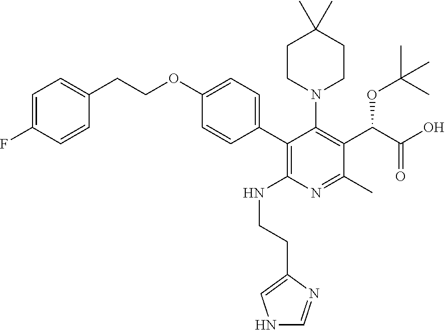 [SMILES: Cc1nc(NCCc2c[nH]cn2)c(-c2ccc(OCCc3ccc(F)cc3)cc2)c(N2CCC(C)(C)CC2)c1[C@H](OC(C)(C)C)C(=O)O]

### (S)-2-(6-((2-(H-Imidazol-4-yl)ethyl)amino)-4-(4, 4-dimethylpiperidin-1-yl)-5-(4-(4-fluorophenethoxy)phenyl)-2-methylpyridin-3-yl)-2-(tert-butoxy)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol) and 2-(1H-imidazol-4-yl)ethanamine (0.012 g, 0.108 mmol) in NMP (1 mL) was heated at 180° C. for 2 h. Ethanol (0.5 mL) and 5 M NaOH (0.054 mL, 0.271 mmol) were added and the mixture was heated at 80° C. for 4.5 h, cooled to ambient temperature, and filtered. The crude mixture was purified via preparative HPLC to afford the desired product (4.8 mg, 26%). LCMS (M+1)=658.3.

### Example 33

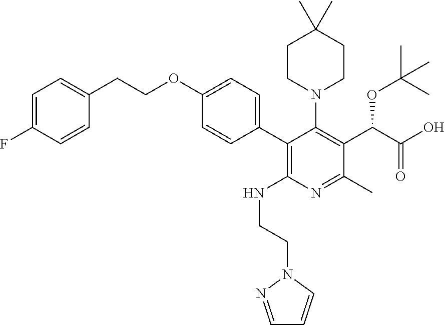 [SMILES: Cc1nc(NCCn2cccn2)c(-c2ccc(OCCc3ccc(F)cc3)cc2)c(N2CCC(C)(C)CC2)c1[C@H](OC(C)(C)C)C(=O)O]

### (S)-2-(6-((2-(1H-Pyrazol-1-yl)ethyl)amino)-4-(4, 4-dimethylpiperidin-1-yl)-5-(4-(4-fluorophenethoxy)phenyl)-2-methylpyridin-3-yl)-2-(tert-butoxy)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol) and 2-(1H-pyrazol-1-yl)ethanamine (0.012 g, 0.108 mmol) in NMP (1 mL) was heated at 180° C. for 2 h. Ethanol (0.5 mL) and 5 M NaOH (0.054 mL, 0.271 mmol) were added and the mixture was heated at 80° C. for 4.5 h, cooled to ambient temperature, and filtered. The crude mixture was purified via preparative HPLC to afford the desired product (6.7 mg, 38%). LCMS (M+1)=658.3.

### Example 34

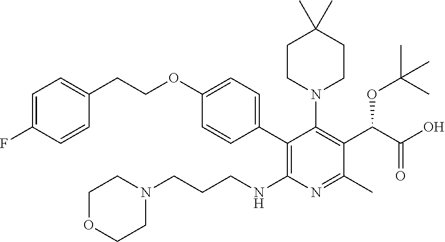 [SMILES: Cc1nc(NCCCN2CCOCC2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-((3-morpholinopropyl)amino)pyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol) and 3-morpholinopropan-1-amine (0.016 g, 0.108 mmol) in NMP (1 mL) was heated at 180° C. for 2 h. Ethanol (0.5 mL) and 5 M NaOH (0.054 mL, 0.271 mmol) were added and the mixture was heated at 80° C. for 4.5 h, cooled to ambient temperature, and filtered. The crude mixture was purified via preparative HPLC to afford the desired product (4.1 mg, 22%). LCMS (M+1)=691.4.

### Example 35

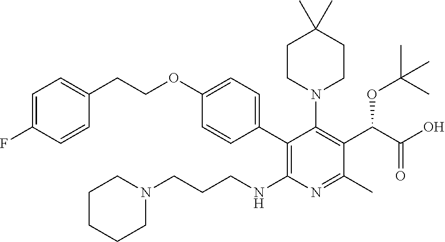 [SMILES: Cc1nc(NCCCN2CCCCC2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-((3-(piperidin-1-yl)propyl)amino)pyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol) and 3-(piperidin-1-yl)propan-1-amine (0.015 g, 0.108 mmol) in NMP (1 mL) was heated at 180° C. for 2 h. Ethanol (0.5 mL) and 5 M NaOH (0.054 mL, 0.271 mmol) were added and the mixture was heated at 80° C. for 4.5 h, cooled to ambient temperature, and filtered. The crude mixture was purified via preparative HPLC to afford the desired product (4.2 mg, 21%). LCMS (M+1)=689.4.

### Example 36

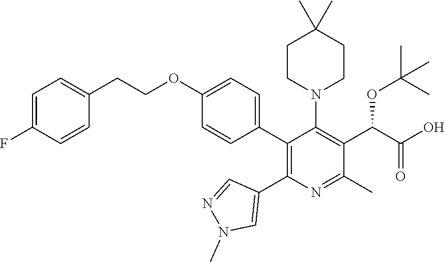 [SMILES: Cc1nc(-c2cnn(C)c2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-(1-methyl-H-pyrazol-4-yl)pyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.03 g, 0.041 mmol) 1-methyl-4-(4,4,5,5-tetramethyl-1,3,2-dioxaborolan-2-yl)-1H-pyrazole (0.017 g, 0.081 mmol), Pd(Ph₃P)₄ (9.38 mg, 8.12 μmol), and 2 M Na₂CO₃ (0.051 ml, 0.102 mmol) in toluene (1 mL) and ethanol (1 mL) was degassed. The reaction was then heated at 90° C. in a sealed tube for 2 h. After cooling to ambient temperature, the reaction mixture was filtered through a pad of celite and concentrated in vacuo. The residue was taken up in ethanol (1 mL) and 5 M NaOH (0.081 mL, 0.406 mmol). The mixture was heated at 80° C. for 3 h, cooled to ambient temperature, and filtered. The crude mixture was purified via preparative HPLC to afford the desired product (15.7 mg, 62%). ¹H NMR (500 MHz, DMSO-d₆) δ 7.39 (dd, J=8.3, 5.7 Hz, 2H), 7.28 (s, 1H), 7.22-7.07 (m, 5H), 7.00 (dd, J=8.4, 2.6 Hz, 1H), 6.57 (s, 1H), 5.89 (br. s., 1H), 4.33-4.22 (m, 2H), 3.69 (s, 2H), 3.08 (t, J=6.8 Hz, 2H), 2.84 (t, J=12.5 Hz, 1H), 2.55 (s, 3H), 2.26 (d, J=11.0 Hz, 1H), 2.03 (t, J=11.4 Hz, 1H), 1.55-1.45 (m, 1H), 1.37-1.27 (m, 1H), 1.20-1.13 (m, 10H), 1.03 (d, J=11.4 Hz, 1H), 0.86 (s, 3H), 0.60 (s, 3H). LCMS (M+1)=629.3.

### Example 37

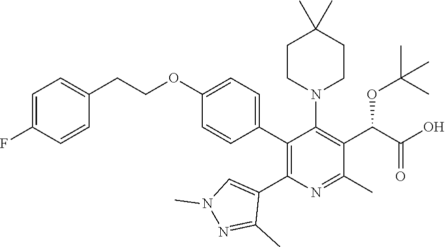 [SMILES: Cc1nn(C)cc1-c1nc(C)c([C@H](OC(C)(C)C)C(=O)O)c(N2CCC(C)(C)CC2)c1-c1ccc(OCCc2ccc(F)cc2)cc1]

### (S)-2-(tert-Butoxy)-2-(6-(1, 3-dimethyl-1H-pyrazol-4-yl)-4-(4, 4-dimethylpiperidin-1-yl)-5-(4-(4-fluorophenethoxy)phenyl)-2-methylpyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.025 g, 0.034 mmol), 1,3-dimethyl-4-(4,4,5,5-tetramethyl-1,3,2-dioxaborolan-2-yl)-1H-pyrazole (0.015 g, 0.068 mmol), Pd(Ph₃P)₄ (7.82 mg, 6.77 μmol), and 2 M Na₂CO₃ (0.042 ml, 0.085 mmol) in toluene (1 mL) and ethanol (1 mL) was degassed. The reaction was heated at 90° C. in a sealed tube for 2 h. After cooling to ambient temperature, the reaction reaction mixture was filtered through a pad of celite and concentrated in vacuo. The residue was taken up in ethanol (1 mL). 5 M NaOH (0.068 mL, 0.338 mmol) was added and the mixture was heated at 80° C. for 3 h, cooled to ambient temperature, and filtered. The crude mixture was purified via preparative HPLC to afford the desired product (14.7 mg, 68%). ¹H NMR (500 MHz, DMSO-d₆) δ 7.37 (dd, J=8.4, 5.5 Hz, 2H), 7.22-7.11 (m, 3H), 7.05 (d, J=8.1 Hz, 1H), 6.99-6.94 (m, 1H), 6.92-6.87 (m, 1H), 6.47 (s, 1H), 5.88 (br. s., 1H), 4.29-4.18 (m, 2H), 3.51 (s, 1H), 3.05 (t, J=6.8 Hz, 2H), 2.86 (t, J=12.7 Hz, 1H), 2.56 (s, 5H), 2.23-2.13 (m, 4H), 1.98-1.90 (m, 3H), 1.52 (br. s., 1H), 1.37-1.26 (m, 1H), 1.16 (s, 10H), 1.02 (d, J=11.7 Hz, 1H), 0.86 (s, 3H), 0.61 (s, 3H). LCMS (M+1)=643.4.

### Example 38

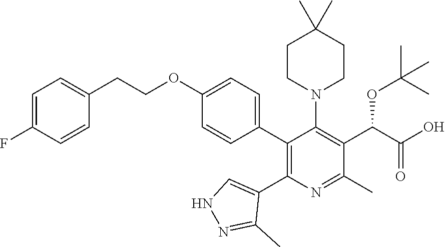 [SMILES: Cc1n[nH]cc1-c1nc(C)c([C@H](OC(C)(C)C)C(=O)O)c(N2CCC(C)(C)CC2)c1-c1ccc(OCCc2ccc(F)cc2)cc1]

### (S)-2-(tert-Butoxy)-2-(4-(4, 4-dimethylpiperidin-1-yl)-5-(4-(4-fluorophenethoxy)phenyl)-2-methyl-6-(3-methyl-1H-pyrazol-4-yl)pyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.025 g, 0.034 mmol), 3-methyl-4-(4,4,5,5-tetramethyl-1,3,2-dioxaborolan-2-yl)-1H-pyrazole (0.014 g, 0.068 mmol), Pd(Ph₃P)₄ (7.82 mg, 6.77 μmol), and 2 M Na₂CO₃ (0.042 ml, 0.085 mmol) in toluene (1 mL) and ethanol (1 mL) was degassed. The reaction was heated at 90° C. in a sealed tube for 2 h. The reaction mixture was filtered through a pad of celite and concentrated in vacuo. The residue was taken up in ethanol (1 mL). 5 M NaOH (0.068 mL, 0.338 mmol) was added and the mixture was heated at 80° C. for 3 h, cooled to ambient temperature, and filtered. The crude mixture was then purified via preparative HPLC to afford the desired product (3.1 mg, 15%). ¹H NMR (500 MHz, DMSO-d₆) δ 7.37 (dd, J=8.4, 5.9 Hz, 2H), 7.15 (q, J=8.9 Hz, 3H), 7.08-7.01 (m, 1H), 6.95 (d, J=8.4 Hz, 1H), 6.90-6.84 (m, 1H), 6.32 (s, 1H), 5.79 (br. s., 1H), 4.27-4.16 (m, 2H), 3.05 (t, J=6.8 Hz, 2H), 2.88-2.79 (m, 1H), 2.55 (s, 6H), 2.28 (s, 3H), 1.91 (s, 2H), 1.53 (br. s., 1H), 1.31 (br. s., 1H), 1.20-1.11 (m, 10H), 1.01 (d, J=12.5 Hz, 1H), 0.85 (s, 3H), 0.61 (s, 3H). LCMS (M+1)=629.3.

### Example 39

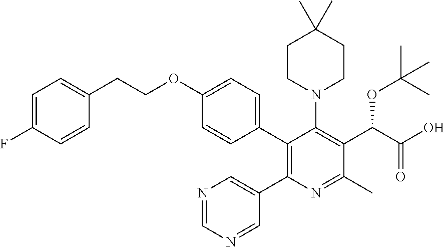 [SMILES: Cc1nc(-c2cncnc2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-(pyrimidin-5-yl)pyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.025 g, 0.034 mmol), pyrimidin-5-ylboronic acid (8.39 mg, 0.068 mmol), Pd(Ph₃P)₄ (7.82 mg, 6.77 μmol), and 2 M Na₂CO₃ (0.042 ml, 0.085 mmol) in toluene (1 mL) and ethanol (1 mL) was degassed. The reaction was heated at 90° C. in a sealed tube for 2 h. The reaction mixture was filtered through a pad of celite and concentrated in vacuo. The residue was taken up in ethanol (1 mL). 5 M NaOH (0.068 ml, 0.338 mmol) was added and the mixture was heated at 80° C. for 3 h, cooled to ambient temperature, and filtered. The crude mixture was purified via preparative HPLC to afford the desired product (1.2 mg, 5%). LCMS (M+1)=627.3.

### Example 40

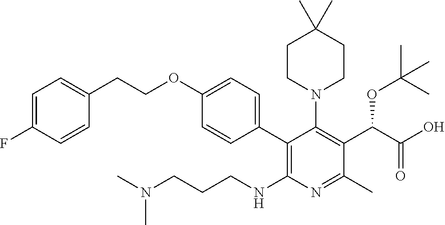 [SMILES: Cc1nc(NCCCN(C)C)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(6-((3-(dimethylamino)propyl)amino)-4-(4, 4-dimethylpiperidin-1-yl)-5-(4-(4-fluorophenethoxy)phenyl)-2-methylpyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol) and N1,N1-dimethylpropane-1,3-diamine (0.011 g, 0.108 mmol) in NMP (1 mL) was heated at 180° C. for 2 h. Ethanol (0.5 mL) and 5 M NaOH (0.054 mL, 0.271 mmol) were added and the mixture was heated at 80° C. for 4.5 h, cooled to ambient temperature, and filtered. The crude mixture was purified via preparative HPLC to afford the desired product (1.4 mg, 8%). LCMS (M+1)=649.4.

### Example 41

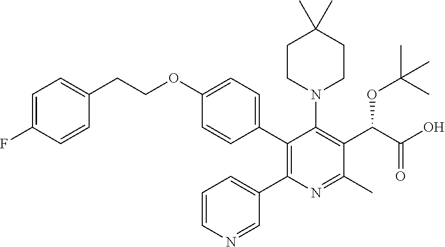 [SMILES: Cc1nc(-c2cccnc2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-3-(4-(4-fluorophenethoxy)phenyl)-6-methyl-[2, 3′-bipyridin]-5-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.03 g, 0.041 mmol), pyridin-3-ylboronic acid (9.98 mg, 0.081 mmol), Pd(Ph₃P)₄ (9.38 mg, 8.12 μmol), and 2 M Na₂CO₃ (0.051 ml, 0.102 mmol) in toluene (1 mL) and ethanol (1 mL) was degassed. The reaction tube was heated at 90° C. in a sealed tube for 2 h. The reaction mixture was filtered through a pad of celite and concentrated in vacuo. The residue was taken up in ethanol (1 mL). 5 M NaOH (0.081 ml, 0.406 mmol) was added and the mixture was heated at 80° C. for 3 h, cooled to ambient temperature and filtered. The crude mixture was purified via preparative HPLC to afford the desired product (6.4 mg, 25%). ¹H NMR (500 MHz, DMSO-d₆) δ 8.67-8.32 (m, 2H), 7.77 (br d, J=7.7 Hz, 1H), 7.51-6.66 (m, 10H), 5.80 (br s, 1H), 4.21-4.05 (m, 2H), 3.00 (brt, J=6.6 Hz, 2H), 2.64 (s, 3H), 2.55 (s, 3H), 1.19 (s, 12H), 0.78 (br s, 6H). LCMS (M+1)=626.3.

### Example 42

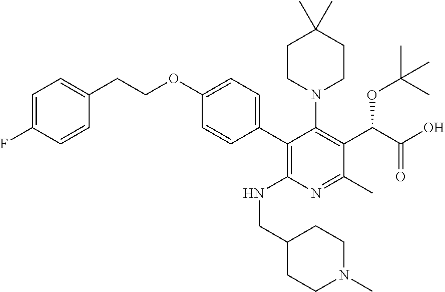 [SMILES: Cc1nc(NCC2CCN(C)CC2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-(((1-methylpiperidin-4-yl)methyl)amino)pyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol) and (1-methylpiperidin-4-yl)methanamine (0.014 g, 0.108 mmol) in NMP (1 mL) was heated at 180° C. for 2 h. Ethanol (0.5 mL) and 5 M NaOH (0.054 mL, 0.271 mmol) were added and the mixture was heated at 80° C. for 4.5 h, cooled to ambient temperature, and filtered. The crude mixture was purified via preparative HPLC to afford the desired product (13.6 mg, 73%). LCMS (M+1)=649.4.

### Example 43

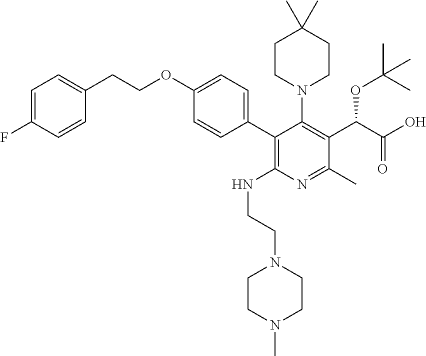 [SMILES: Cc1nc(NCCN2CCN(C)CC2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-((2-(4-methylpiperazin-1-yl)ethyl)amino)pyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol) and 2-(4-methylpiperazin-1-yl)ethanamine (0.016 g, 0.108 mmol) in NMP (1 mL) was heated at 180° C. for 2 h. Ethanol (0.5 mL) and 5 M NaOH (0.054 mL, 0.271 mmol) were added and the mixture was heated at 80° C. for 4.5 h, cooled to ambient temperature, and filtered. The crude mixture was purified via preparative HPLC to afford the desired product (11.8 mg, 62%). LCMS (M+1)=690.2.

### Example 44

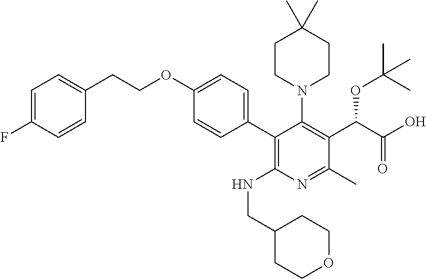 [SMILES: Cc1nc(NCC2CCOCC2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-(((tetrahydro-2H-pyran-4-yl)methyl)amino)pyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol) and (tetrahydro-2H-pyran-4-yl)methanamine (0.012 g, 0.108 mmol) in NMP (1 mL) was heated at 180° C. for 2 h. Ethanol (0.5 mL) and 5 M NaOH (0.054 mL, 0.271 mmol) were added and the mixture was heated at 80° C. for 4.5 h, cooled to ambient temperature, and filtered. The crude mixture was purified via preparative HPLC to afford the desired product (11.6 mg, 65%). ¹H NMR (500 MHz, DMSO-d₆) δ 7.38 (dd, J=8.4, 5.9 Hz, 2H), 7.23-6.98 (m, 6H), 5.73 (br. s., 1H), 4.45 (br. s., 1H), 4.32-4.18 (m, 2H), 3.84-3.72 (m, 2H), 3.26-3.01 (m, 4H), 2.75 (t, J=12.7 Hz, 1H), 2.55 (s, 2H), 2.37-2.28 (m, 3H), 2.16 (br. s., 1H), 1.97-1.87 (m, 2H), 1.72 (br. s., 1H), 1.56-1.24 (m, 4H), 1.23-0.95 (m, 12H), 0.88-0.79 (m, 3H), 0.59 (s, 3H). LCMS (M+1)=662.4.

### Example 45

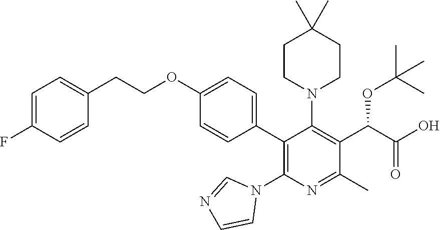 [SMILES: Cc1nc(-n2ccnc2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-6-(1H-imidazol-1-yl)-2-methylpyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol) and 1H-imidazole (0.018 g, 0.271 mmol) in NMP (1 mL) was heated at 180° C. for 2 h. Ethanol (0.5 mL) and 5 M NaOH (0.054 mL, 0.271 mmol) were added and the mixture was heated at 80° C. for 4.5 h, cooled to ambient temperature, and filtered. The crude mixture was purified via preparative HPLC to afford the desired product (5.7 mg, 34%). ¹H NMR (500 MHz, DMSO-d₆) δ 7.49 (s, 1H), 7.36 (dd, J=8.4, 5.9 Hz, 2H), 7.26 (d, J=8.4 Hz, 1H), 7.14 (t, J=8.8 Hz, 2H), 7.05-6.98 (m, 3H), 6.87-6.82 (m, 1H), 6.76 (s, 1H), 5.86 (br. s., 1H), 4.25-4.12 (m, 2H), 3.03 (t, J=6.8 Hz, 2H), 2.91 (br. s., 1H), 2.55 (s, 2H), 2.21 (br. s., 1H), 1.92 (s, 2H), 1.55 (br. s., 1H), 1.36-1.21 (m, 2H), 1.18 (s, 9H), 1.05 (d, J=6.2 Hz, 1H), 0.87 (br. s., 3H), 0.64 (br. s., 3H). LCMS (M+1)=615.3.

### Example 46

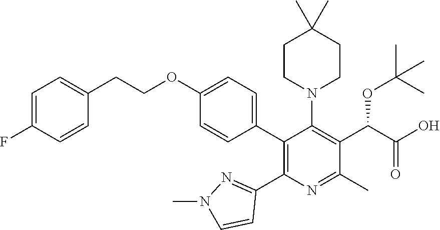 [SMILES: Cc1nc(-c2ccn(C)n2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-(1-methyl-1H-pyrazol-3-yl)pyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.025 g, 0.034 mmol), 1-methyl-3-(4,4,5,5-tetramethyl-1,3,2-dioxaborolan-2-yl)-1H-pyrazole (0.014 g, 0.068 mmol), Pd(Ph₃P)₄ (7.82 mg, 6.77 μmol), and 2 M Na₂CO₃ (0.042 ml, 0.085 mmol) in toluene (1 mL) and ethanol (1 mL) was degassed. The reaction was heated at 90° C. for 2 h. After cooling to ambient temperature, the reaction mixture was filtered through a pad of celite and concentrated in vacuo. The residue was taken up in ethanol (1 mL). 5 M NaOH (0.068 mL, 0.338 mmol) was added and the mixture was heated at 80° C. for 3 h, cooled to ambient temperature and filtered. The crude mixture was purified via preparative HPLC to afford the desired product (14.6 mg, 68%). ¹H NMR (500 MHz, DMSO-d₆) δ 7.40-7.33 (m, 3H), 7.14 (t, J=8.6 Hz, 3H), 7.01-6.94 (m, 2H), 6.83 (dd, J=8.6, 2.4 Hz, 1H), 5.95 (br. s., 1H), 5.51 (d, J=2.2 Hz, 1H), 4.25-4.13 (m, 2H), 3.72 (s, 3H), 3.23 (br. s., 1H), 3.03 (t, J=6.6 Hz, 2H), 2.89 (t, J=11.9 Hz, 1H), 2.55 (s, 4H), 2.20 (d, J=11.4 Hz, 1H), 1.99-1.90 (m, 2H), 1.51 (br. s., 1H), 1.30 (d, J=16.1 Hz, 1H), 1.24-1.13 (m, 11H), 1.03 (d, J=12.1 Hz, 1H), 0.86 (s, 3H), 0.61 (s, 3H). LCMS (M+1)=629.3.

### Example 47

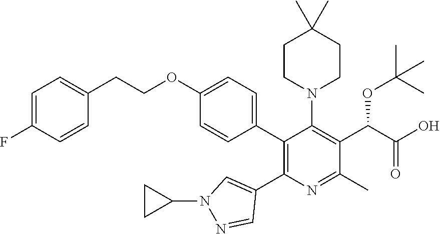 [SMILES: Cc1nc(-c2cnn(C3CC3)c2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(6-(1-cyclopropyl-H-pyrazol-4-yl)-4-(4, 4-dimethylpiperidin-1-yl)-5-(4-(4-fluorophenethoxy)phenyl)-2-methylpyridin-3-yl)acetic acid

A degassed mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol), 1-cyclopropyl-4-(4,4,5,5-tetramethyl-1,3,2-dioxaborolan-2-yl)-1H-pyrazole (0.013 g, 0.054 mmol), Pd(Ph₃P)₄ (6.26 mg, 5.41 μmol), and 2 M Na₂CO₃ (0.034 ml, 0.068 mmol) in toluene and ethanol was heated at 90° C. for 2 h. The reaction mixture was allowed to cool to ambient temperature and then filtered through a pad of celite and concentrated in vacuo. The residue was taken up in ethanol (1 mL). 5 M NaOH (0.054 ml, 0.271 mmol) was added and the mixture was heated at 80° C. for 3 h, cooled to ambient temperature, and filtered. The crude mixture was purified via preparative HPLC to afford the desired product (9.6 mg, 53%). LCMS (M+1)=655.3.

### Example 48

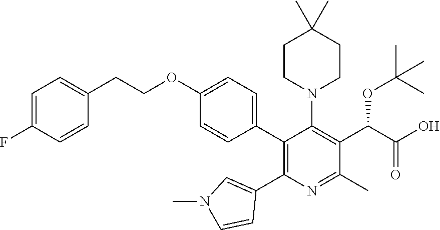 [SMILES: Cc1nc(-c2ccn(C)c2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-(1-methyl-1H-pyrrol-3-yl)pyridin-3-yl)acetic acid

A degassed mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol), 1-methyl-3-(4,4,5,5-tetramethyl-1,3,2-dioxaborolan-2-yl)-1H-pyrrole (0.011 g, 0.054 mmol), Pd(Ph₃P)₄ (6.26 mg, 5.41 μmol), and 2 M Na₂CO₃ (0.034 ml, 0.068 mmol) in toluene and ethanol was heated at 90° C. for 2 h. The reaction mixture was filtered through a pad of celite and concentrated in vacuo. The residue was taken up in ethanol (1 mL), 5 M NaOH (0.054 ml, 0.271 mmol) was added, and the mixture was heated at 80° C. for 3 h. After cooling to ambient temperature, the reaction was filtered. The crude mixture was purified via preparative HPLC to afford the desired product (7.5 mg, 43%). LCMS (M+1)=628.2.

### Example 49

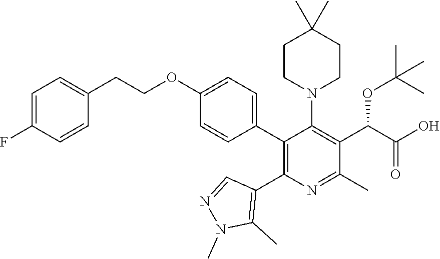 [SMILES: Cc1nc(-c2cnn(C)c2C)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(6-(1,5-dimethyl-1H-pyrazol-4-yl)-4-(4, 4-dimethylpiperidin-1-yl)-5-(4-(4-fluorophenethoxy)phenyl)-2-methylpyridin-3-yl)acetic acid

A degassed mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol), 1,5-dimethyl-4-(4,4,5,5-tetramethyl-1,3,2-dioxaborolan-2-yl)-1H-pyrazole (0.012 g, 0.054 mmol), Pd(Ph₃P)₄ (6.26 mg, 5.41 μmol), and 2 M Na₂CO₃ (0.034 ml, 0.068 mmol) in toluene (1 mL) and ethanol (1 mL) was heated at 90° C. for 2 h. The reaction mixture was filtered through a pad of celite and concentrated in vacuo. The residue was taken up in ethanol (1 mL) and 5 M NaOH (0.054 ml, 0.271 mmol) was added. The mixture was heated at 80° C. for 3 h, cooled to ambient temperature, and filtered. The crude mixture was purified via preparative HPLC to afford the desired product (6.1 mg, 35%). LCMS (M+1)=643.3.

### Example 50

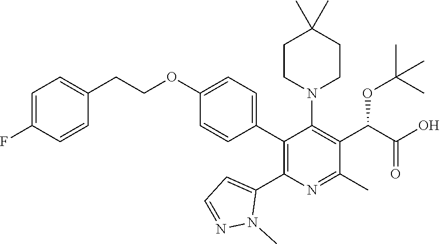 [SMILES: Cc1nc(-c2ccnn2C)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-(1-methyl-1H-pyrazol-5-yl)pyridin-3-yl)acetic acid

A degassed mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol), 1-methyl-5-(4,4,5,5-tetramethyl-1,3,2-dioxaborolan-2-yl)-1H-pyrazole (0.011 g, 0.054 mmol), Pd(Ph₃P)₄ (6.26 mg, 5.41 μmol), and 2 M Na₂CO₃ (0.034 ml, 0.068 mmol) in toluene (1 mL) and ethanol (1 mL) was heated at 90° C. for 2 h. The reaction mixture was filtered through a plug of celite and concentrated in vacuo. The residue was taken up in ethanol (1 mL) and 5 M NaOH (0.054 mL, 0.271 mmol) was added. The mixture was heated at 80° C. for 3 h, cooled to ambient temperature and filtered. The crude mixture was purified via preparative HPLC to afford the desired product (8.8 mg, 52%). ¹H NMR (500 MHz, DMSO-d₆) δ 7.35 (dd, J=8.4, 5.9 Hz, 1H), 7.21-7.10 (m, 2H), 7.01-6.90 (m, 1H), 6.80 (dd, J=8.4, 2.6 Hz, 1H), 5.91 (br. s., 1H), 5.57 (d, J=1.8 Hz, 1H), 4.24-4.09 (m, 2H), 3.70 (s, 2H), 3.02 (t, J=6.6 Hz, 2H), 2.56-2.54 (m, 8H), 2.19 (br. s., 1H), 1.54 (br. s., 1H), 1.38-1.20 (m, 2H), 1.17 (s, 9H), 1.05 (d, J=5.9 Hz, 1H), 0.87 (br. s., 3H), 0.63 (br. s., 3H). LCMS (M+1)=629.3.

### Example 51

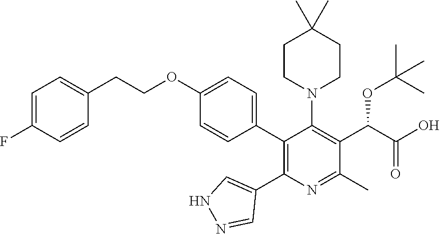 [SMILES: Cc1nc(-c2cn[nH]c2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-(1H-pyrazol-4-yl)pyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.025 g, 0.034 mmol), 4-(4,4,5,5-tetramethyl-1,3,2-dioxaborolan-2-yl)-1-trityl-1H-pyrazole (0.030 g, 0.068 mmol), Pd(Ph₃P)₄ (7.82 mg, 6.77 μmol), and 2 M Na₂CO₃ (0.042 ml, 0.085 mmol) in toluene (1 mL) and ethanol (1 mL) was heated at 90° C. for 2 h. The reaction mixture was filtered through a pad of celite and concentrated in vacuo. The residue was taken up in ethanol (1 mL) and 5 M NaOH (0.068 ml, 0.338 mmol) was added. The mixture was heated at 80° C. for 3 h, cooled to ambient temperature, and concentrated in vacuo. The residue was taken up in methanol (1 mL) and DCM (1 mL), 2 drops of conc. HCl was added and the mixture was stirred at 40° C. for 3 d. The reaction mixture was concentrated, taken up in ethanol, and filtered. The crude mixture was purified via preparative HPLC to afford the desired product (9.1 mg, 44%). ¹H NMR (500 MHz, DMSO-d₆) δ 7.39 (dd, J=8.4, 5.9 Hz, 1H), 7.22-7.07 (m, 2H), 7.03-6.95 (m, 1H), 5.90 (br. s., 1H), 4.32-4.20 (m, 2H), 3.08 (t, J=6.8 Hz, 2H), 2.84 (t, J=12.1 Hz, 1H), 2.55 (s, 4H), 2.27 (d, J=10.3 Hz, 1H), 2.08-2.00 (m, 1H), 1.91 (s, 2H), 1.50 (br. s., 1H), 1.36-1.27 (m, 1H), 1.21-1.12 (m, 10H), 1.04 (d, J=11.7 Hz, 1H), 0.86 (s, 3H), 0.60 (s, 3H). LCMS (M+1)=615.3.

### Example 52

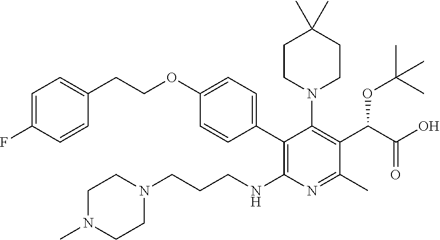 [SMILES: Cc1nc(NCCCN2CCN(C)CC2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-((3-(4-methylpiperazin-1-yl)propyl)amino)pyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol) and 3-(4-methylpiperazin-1-yl)propan-1-amine (0.017 g, 0.108 mmol) in NMP (1 mL) was heated at 180° C. for 3 h. Ethanol (1 mL) and 5 M NaOH (0.054 mL, 0.271 mmol) were added. The mixture was heated at 80° C. for 3 h, cooled to ambient temperature, and filtered. The crude mixture was purified via preparative HPLC to afford the desired product (10.5 mg, 55%). ¹H NMR (500 MHz, DMSO-d₆) δ 7.45-7.33 (m, 1H), 7.21-7.01 (m, 3H), 5.71 (br. s., 1H), 4.32-4.15 (m, 3H), 3.34-3.11 (m, 3H), 3.05 (t, J=6.6 Hz, 3H), 2.74 (t, J=11.2 Hz, 1H), 2.39-1.82 (m, 23H), 1.61-1.39 (m, 4H), 1.27 (d, J=16.5 Hz, 1H), 1.19-1.10 (m, 9H), 0.99 (d, J=12.5 Hz, 1H), 0.83 (s, 3H), 0.59 (s, 3H). LCMS (M+1)=704.4.

### Example 53

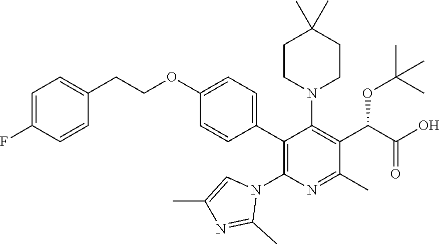 [SMILES: Cc1cn(-c2nc(C)c([C@H](OC(C)(C)C)C(=O)O)c(N3CCC(C)(C)CC3)c2-c2ccc(OCCc3ccc(F)cc3)cc2)c(C)n1]

### (S)-2-(tert-Butoxy)-2-(6-(2, 4-dimethyl-1H-imidazol-1-yl)-4-(4, 4-dimethylpiperidin-1-yl)-5-(4-(4-fluorophenethoxy)phenyl)-2-methylpyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol) and 2,4-dimethyl-1H-imidazole (0.026 g, 0.271 mmol) in NMP (1 mL) was heated at 180° C. for 2 h. Ethanol (0.5 mL) and 5 M NaOH (0.054 mL, 0.271 mmol) were added. The mixture was heated at 80° C. for 3 h, cooled to ambient temperature, and filtered. The crude mixture was purified via preparative HPLC to afford the desired product (7.0 mg, 40%). LCMS (M+1)=643.3.

### Example 54

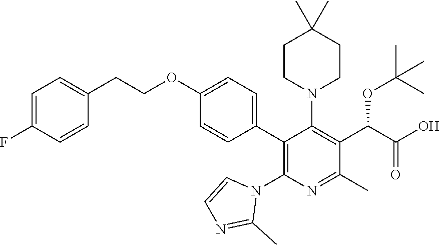 [SMILES: Cc1nc(-n2ccnc2C)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-(2-methyl-1H-imidazol-1-yl)pyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol) and 2-methyl-1H-imidazole (0.022 g, 0.271 mmol) in NMP (1 mL) was heated at 180° C. for 2 h. Ethanol (0.5 mL) and 5 M NaOH (0.054 mL, 0.271 mmol) were added. The mixture was heated at 80° C. for 4.5 h, cooled to ambient temperature, and filtered. The crude mixture was purified via preparative HPLC to afford the desired product (7.2 mg, 42%). ¹H NMR (500 MHz, DMSO-d₆) δ 7.35 (dd, J=8.6, 5.7 Hz, 1H), 7.18-7.10 (m, 1H), 6.98-6.86 (m, 1H), 6.76 (d, J=6.6 Hz, 1H), 6.56 (s, 1H), 5.74 (br. s., 1H), 4.15 (dt, J=13.5, 6.6 Hz, 3H), 3.01 (t, J=6.8 Hz, 3H), 2.89 (br. s., 1H), 2.55 (s, 1H), 2.19 (br. s., 1H), 2.01 (s, 5H), 1.92 (s, 3H), 1.56 (br. s., 1H), 1.25 (br. s., 2H), 1.15 (s, 9H), 1.06 (br. s., 1H), 0.87 (br. s., 3H), 0.65 (br. s., 3H). LCMS (M+1)=629.2.

### Example 55 Example 56

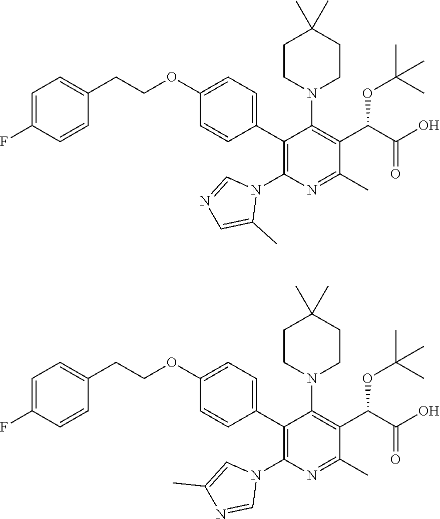 [SMILES: Cc1nc(-n2cncc2C)c(-c2ccc(OCCc3ccc(F)cc3)cc2)c(N2CCC(C)(C)CC2)c1[C@H](OC(C)(C)C)C(=O)O ; Cc1cn(-c2nc(C)c([C@H](OC(C)(C)C)C(=O)O)c(N3CCC(C)(C)CC3)c2-c2ccc(OCCc3ccc(F)cc3)cc2)cn1]

### (S)-2-(tert-Butoxy)-2-(4-(4, 4-dimethylpiperidin-1-yl)-5-(4-(4-fluorophenethoxy)phenyl)-2-methyl-6-(5-methyl-1H-imidazol-1-yl)pyridin-3-yl)acetic acid and (S)-2-(tert-butoxy)-2-(4-(4, 4-dimethylpiperidin-1-yl)-5-(4-(4-fluorophenethoxy)phenyl)-2-methyl-6-(4-methyl-1H-imidazol-1-yl)pyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol) and 4-methyl-1H-imidazole (0.022 g, 0.271 mmol) in NMP (1 mL) was heated at 180° C. for 2 h. Ethanol (0.5 mL) and 5 M NaOH (0.054 mL, 0.271 mmol) were added. The mixture was heated at 80° C. for 4.5 h, cooled to ambient temperature, and filtered. The crude mixture was purified via preparative HPLC to afford the desired products (2.7 mg, 16%) and (6.4 mg, 38%). Isomer regiochemistry was not assigned. First eluting isomer, LCMS (M+1)=629.2. Second eluting isomer, LCMS (M+1)=629.2.

### Example 57

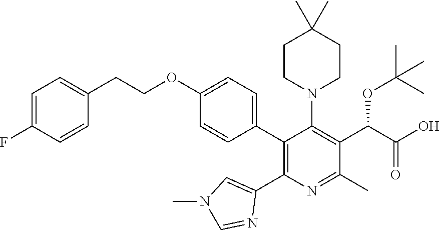 [SMILES: Cc1nc(-c2cn(C)cn2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-(1-methyl-1H-imidazol-4-yl)pyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.03 g, 0.041 mmol), 1-methyl-4-(tributylstannyl)-1H-imidazole (0.022 mL, 0.063 mmol), Pd(Ph₃P)₄ (9.38 mg, 8.12 μmol), and a catalytic amount of lithium chloride in DME (1 mL) was degassed. The reaction was heated at 110° C. for 1 h. Upon cooling to ambient temperature, the reaction mixture was filtered through a pad of celite and concentrated in vacuo. The residue was taken up in ethanol (1 mL) and 5 M NaOH (0.081 mL, 0.406 mmol) was added. The mixture was heated at 80° C. for 3 h, cooled to ambient temperature, and filtered. The crude mixture was purified via preparative HPLC to afford the desired product (11.6 mg, 45%). ¹H NMR (500 MHz, DMSO-d₆) δ 7.40-7.31 (m, 3H), 7.19-6.96 (m, 5H), 6.87 (dd, J=8.4, 2.6 Hz, 1H), 6.41 (s, 1H), 5.91 (br s, 1H), 4.29-4.14 (m, 2H), 3.05 (t, J=6.6 Hz, 2H), 2.89-2.80 (m, 1H), 2.56-2.56 (m, 1H), 2.56 (s, 1H), 2.49 (s, 3H), 2.24-2.17 (m, 1H), 1.96-1.87 (m, 2H), 1.55-1.46 (m, 1H), 1.36-1.26 (m, 1H), 1.22-1.11 (m, 11H), 1.05-0.99 (m, 1H), 0.86 (s, 3H), 0.61 (s, 3H). LCMS (M+1)=629.4.

### Example 58

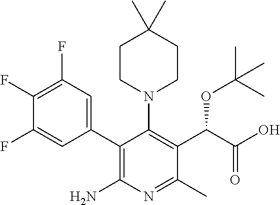 [SMILES: Cc1nc(N)c(-c2cc(F)c(F)c(F)c2)c(N2CCC(C)(C)CC2)c1[C@H](OC(C)(C)C)C(=O)O]

### (S)-2-(6-Amino-4-(4, 4-dimethylpiperidin-1-yl)-2-methyl-5-(3,4, 5-trifluorophenyl)pyridin-3-yl)-2-(tert-butoxy)acetic acid

10 N NaOH (0.1 mL, 1.0 mmol) was added to a solution of (S)-isopropyl 2-(6-amino-4-(4,4-dimethylpiperidin-1-yl)-2-methyl-5-(3,4,5-trifluorophenyl)pyridin-3-yl)-2-(tert-butoxy)acetate (0.02 g, 0.038 mmol) in ethanol (1 mL). The mixture was heated at 60° C. for 18 h, cooled to ambient temperature, and filtered. The crude mixture was purified via preparative HPLC to afford the desired product (10.3 mg, 52%). ¹H NMR (500 MHz, DMSO-d₆) δ 7.38-7.25 (m, 1H), 7.21-7.12 (m, 1H), 5.67 (br. s., 1H), 5.29 (s, 2H), 3.27 (br. s., 1H), 2.76 (t, J=12.1 Hz, 1H), 2.33 (br. s., 1H), 2.29 (s, 3H), 1.98-1.85 (m, 1H), 1.52-1.43 (m, 1H), 1.36-1.26 (m, 1H), 1.20 (d, J=12.8 Hz, 1H), 1.14 (s, 9H), 1.09 (d, J=11.4 Hz, 1H), 0.87 (s, 3H), 0.66 (s, 3H). LCMS (M+1)=480.1.

### Example 59

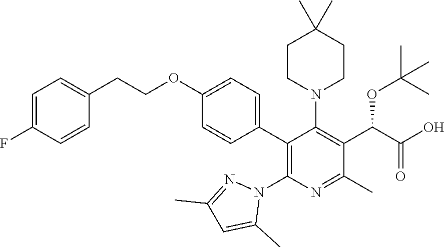 [SMILES: Cc1cc(C)n(-c2nc(C)c([C@H](OC(C)(C)C)C(=O)O)c(N3CCC(C)(C)CC3)c2-c2ccc(OCCc3ccc(F)cc3)cc2)n1]

### (S)-2-(tert-Butoxy)-2-(6-(3,5-dimethyl-1H-pyrazol-1-yl)-4-(4, 4-dimethylpiperidin-1-yl)-5-(4-(4-fluorophenethoxy)phenyl)-2-methylpyridin-3-yl)acetic acid

Acetylacetone (0.015 g, 0.150 mmol) was added to a solution of (S)-isopropyl 2-(tert-butoxy)-2-(4-(4,4-dimethylpiperidin-1-yl)-5-(4-(4-fluorophenethoxy)phenyl)-6-hydrazinyl-2-methylpyridin-3-yl)acetate (0.031 g, 0.050 mmol) in AcOH (0.5 mL) and heated at 80° C. for 1 h. The reaction mixture was diluted with ethyl acetate, washed with 10% K₂CO₃, dried (Na₂SO₄), and concentrated in vacuo. The residue was taken up in MeOH (1.5 mL) and 10 N NaOH, heated at 70° C. for 18 h, cooled to ambient temperature, and filtered. The crude mixture was purified via preparative HPLC to afford the desired product (18.2 mg, 57%). ¹H NMR (500 MHz, DMSO-d₆) δ 7.34 (dd, J=8.4, 5.9 Hz, 2H), 7.13 (t, J=9.0 Hz, 2H), 7.07 (d, J=9.2 Hz, 1H), 6.91 (d, J=6.6 Hz, 2H), 6.73 (d, J=8.4 Hz, 1H), 5.78 (br. s., 1H), 5.67 (s, 1H), 4.22-4.06 (m, 2H), 3.52-3.26 (m, 2H), 3.00 (t, J=6.6 Hz, 2H), 2.91 (br. s., 1H), 2.49 (s, 3H), 2.15 (br. s., 1H), 2.01 (s, 3H), 1.81 (s, 3H), 1.55 (br. s., 1H), 1.34-1.21 (m, 2H), 1.14 (s, 9H), 1.05 (br. s., 1H), 0.87 (br. s., 3H), 0.65 (br. s., 3H). LCMS(M+1)=643.3

### Example 60

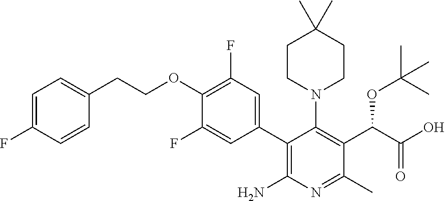 [SMILES: Cc1nc(N)c(-c2cc(F)c(OCCc3ccc(F)cc3)c(F)c2)c(N2CCC(C)(C)CC2)c1[C@H](OC(C)(C)C)C(=O)O]

### (S)-2-(6-Amino-5-(3, 5-difluoro-4-(4-fluorophenethoxy)phenyl)-4-(4, 4-dimethylpiperidin-1-yl)-2-methylpyridin-3-yl)-2-(tert-butoxy)acetic acid

A mixture of (S)-isopropyl 2-(6-amino-5-bromo-4-(4,4-dimethylpiperidin-1-yl)-2-methylpyridin-3-yl)-2-(tert-butoxy)acetate (0.027 g, 0.057 mmol), 2-(3,5-difluoro-4-(4-fluorophenethoxy)phenyl)-4,4,5,5-tetramethyl-1,3,2-dioxaborolane (0.028 g, 0.075 mmol), 2-dicyclohexylphosphino-2′,6′-dimethoxybiphenyl (4.71 mg, 0.011 mmol), palladium(II) acetate (1.289 mg, 5.74 μmol) and 2 M K₃PO₄ (0.086 mL, 0.172 mmol) in dioxane (1 mL) was purged with nitrogen. The reaction was heated at 80° C. for 1 h. The reaction mixture was then filtered through celite, diluted with ethyl acetate, washed with brine, dried (Na₂SO₄), concentrated in vacuo, and purified on silica gel (220 g column) using 0-70% ethyl acetate in hexanes. The desired fractions were concentrated in vacuo give a light orange solid. The solid was taken up in ethanol (1 mL) and 5 M NaOH (0.115 mL, 0.574 mmol) was added. The mixture was heated at 80° C. for 2 h, cooled to ambient temperature, and filtered. The crude mixture was purified via preparative HPLC to afford the desired product (10.2 mg, 29%). ¹H NMR (500 MHz, DMSO-d6) δ 7.38-7.33 (m, 2H), 7.18-7.11 (m, 2H), 7.08 (br d, J=11.4 Hz, 1H), 6.96-6.91 (m, 1H), 5.68-5.63 (m, 1H), 5.21 (s, 2H), 4.37-4.31 (m, 2H), 3.03 (t, J=6.8 Hz, 2H), 2.80-2.72 (m, 1H), 2.28 (s, 4H), 1.98-1.90 (m, 1H), 1.52-1.42 (m, 1H), 1.35-1.26 (m, 1H), 1.22-1.02 (m, 12H), 0.86 (s, 3H), 0.64 (s, 3H). LCMS (M+1)=600.22.

### Example 61

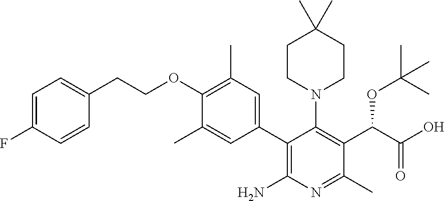 [SMILES: Cc1cc(-c2c(N)nc(C)c([C@H](OC(C)(C)C)C(=O)O)c2N2CCC(C)(C)CC2)cc(C)c1OCCc1ccc(F)cc1]

### (S)-2-(6-Amino-4-(4, 4-dimethylpiperidin-1-yl)-5-(4-(4-fluorophenethoxy)-3, 5-dimethylphenyl)-2-methylpyridin-3-yl)-2-(tert-butoxy)acetic acid

A mixture of (S)-isopropyl 2-(6-amino-5-bromo-4-(4,4-dimethylpiperidin-1-yl)-2-methylpyridin-3-yl)-2-(tert-butoxy)acetate (0.035 g, 0.074 mmol), 2-(4-(4-fluorophenethoxy)-3,5-dimethylphenyl)-4,4,5,5-tetramethyl-1,3,2-dioxaborolane (0.036 g, 0.097 mmol), 2-dicyclohexylphosphino-2′,6′-dimethoxybiphenyl (6.11 mg, 0.015 mmol), palladium(II) acetate (1.670 mg, 7.44 μmol), and 2 M K₃PO₄ (0.112 ml, 0.223 mmol) in dioxane (1 mL) was purged with nitrogen and then heated at 80° C. for 1 h. The reaction mixture was filtered through a pad of celite, diluted with ethyl acetate, washed with brine, dried (Na₂SO₄), concentrated in vacuo, and purified on silica gel (220 g column) using 0-70% ethyl acetate in hexanes. The desired fractions were concentrated in vacuo give a light orange solid. The solid was taken up in ethanol (1 mL) and 5 M NaOH (0.115 mL, 0.574 mmol) was added. The mixture was heated at 80° C. for 2 h, cooled to ambient temperature, and filtered. The crude mixture was purified via preparative HPLC to afford the desired product (14.5 mg, 33%). ¹H NMR (500 MHz, DMSO-d6) δ 7.47-7.32 (m, 2H), 7.23-7.09 (m, 2H), 6.93 (s, 1H), 6.81-6.80 (m, 1H), 6.79 (s, 1H), 5.80-5.64 (m, 1H), 5.02-4.86 (m, 2H), 4.07-3.91 (m, 2H), 3.06 (br t, J=6.4 Hz, 2H), 2.85-2.75 (m, 1H), 2.56 (s, 1H), 2.31-2.22 (m, 3H), 2.20-2.08 (m, 6H), 1.91-1.83 (m, 1H), 1.52-1.40 (m, 1H), 1.33-1.23 (m, 1H), 1.21-1.08 (m, 10H), 0.99 (br d, J=11.0 Hz, 1H), 0.88-0.78 (m, 3H), 0.59 (s, 3H). LCMS (M+1)=592.29.

### Example 62

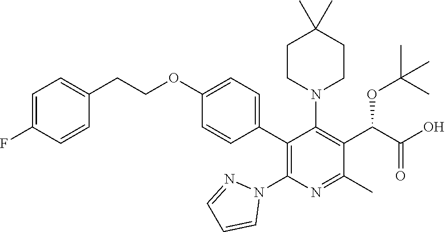 [SMILES: Cc1nc(-n2cccn2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-(1H-pyrazol-1-yl)pyridin-3-yl)acetic acid

1,1,3,3-Tetraethoxypropane (0.024 g, 0.100 mmol) was added to a solution of (S)-isopropyl 2-(tert-butoxy)-2-(4-(4,4-dimethylpiperidin-1-yl)-5-(4-(4-fluorophenethoxy)phenyl)-6-hydrazinyl-2-methylpyridin-3-yl)acetate (0.031 g, 0.050 mmol) in AcOH (0.5 mL). The reaction heated to 80° C. for 1 h. The reaction mixture was diluted with ethyl acetate, washed with 10% K₂CO₃, dried (Na₂SO₄), and concentrated in vacuo. The residue was taken up in MeOH (1.5 mL) and 10 N NaOH (0.15 mL). The reaction heated at 70° C. for 18 h, cooled to ambient temperature, and filtered. The crude mixture was purified via preparative HPLC to afford the desired product (1.4 mg, 4%). LCMS (M+1)=615.1.

### Example 63

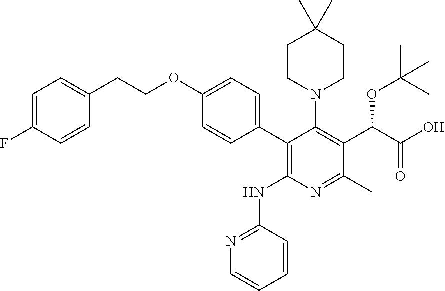 [SMILES: Cc1nc(Nc2ccccn2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-(pyridin-2-ylamino)pyridin-3-yl)acetic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.05 g, 0.083 mmol), 2-chloropyridine (0.011 g, 0.099 mmol), Xantphos (2.87 mg, 3 4.95 μmol), tris(dibenzylideneacetone)dipalladium(0) (1.512 mg, 1.651 μmol), and cesium carbonate (0.038 g, 0.116 mmol) in dioxane (0.825 mL) was degassed and heated at 100° C. for 18 h. The reaction mixture was cooled, filtered through a pad of celite, and concentrated in vacuo. The residue was taken up in methanol (1 mL) and 5 M NaOH (0.132 mL, 0.660 mmol) was added. The mixture was heated at 60° C. for 18 h, cooled to ambient temperature and filtered. The crude mixture was purified via preparative HPLC to afford the desired product (27.1 mg, 50%). ¹H NMR (500 MHz, DMSO-d₆) δ 8.48 (d, J=8.4 Hz, 1H), 8.05 (d, J=4.8 Hz, 1H), 7.70 (t, J=7.9 Hz, 1H), 7.44-7.34 (m, 3H), 7.23-7.13 (m, 5H), 6.91-6.83 (m, 2H), 5.80 (br. s., 1H), 4.29 (q, J=6.6 Hz, 2H), 3.34-3.27 (m, 1H), 3.18 (br. s., 1H), 3.09 (t, J=6.6 Hz, 2H), 2.81 (t, J=12.1 Hz, 1H), 2.49 (s, 3H), 2.26 (d, J=11.0 Hz, 1H), 1.51 (br. s., 1H), 1.37-1.28 (m, 1H), 1.20 (d, J=11.7 Hz, 1H), 1.16 (s, 9H), 1.05 (d, J=12.8 Hz, 1H), 0.86 (s, 3H), 0.62 (s, 3H). LCMS (M+1)=641.1.

### Example 64

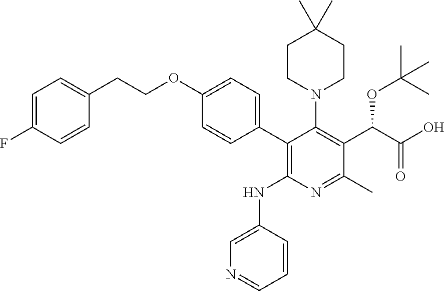 [SMILES: Cc1nc(Nc2cccnc2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-(pyridin-3-ylamino)pyridin-3-yl)acetic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.032 g, 0.053 mmol), 3-bromopyridine (0.012 g, 0.074 mmol), Xantphos (1.834 mg, 3.17 μmol), tris(dibenzylideneacetone)dipalladium(0) (0.967 mg, 1.056 μmol), and cesium carbonate (0.024 g, 0.074 mmol) in dioxane (0.528 mL) was degassed and heated at 100° C. for 4 h. The reaction mixture was cooled, filtered through a pad of celite, and concentrated in vacuo. The residue was taken up in methanol (1 mL) and 5 M NaOH (0.132 mL, 0.660 mmol) was added. The mixture was heated at 60° C. for 18 h, cooled to ambient temperature, and filtered. The crude mixture was purified via preparative HPLC to afford the desired product (4.2 mg, 12%). ¹H NMR (500 MHz, DMSO-d₆) δ 8.63 (br. s., 1H), 8.09-7.98 (m, 2H), 7.44-7.37 (m, 2H), 7.33 (d, J=8.4 Hz, 1H), 7.23-7.06 (m, 6H), 6.73 (s, 1H), 5.73 (br. s., 1H), 4.27 (d, J=11.0 Hz, 2H), 3.12-3.02 (m, 2H), 2.87-2.75 (m, 1H), 2.41 (s, 3H), 2.20 (d, J=11.7 Hz, 1H), 1.96-1.82 (m, 1H), 1.51 (br. s., 1H), 1.31 (br. s., 1H), 1.20 (br. s., 1H), 1.15 (s, 9H), 1.03 (d, J=10.6 Hz, 1H), 0.86 (br. s., 3H), 0.62 (br. s., 3H) [note: 1H of piperidine does not show likely due to water suppression]. LCMS (M+1)=641.1.

### Example 65

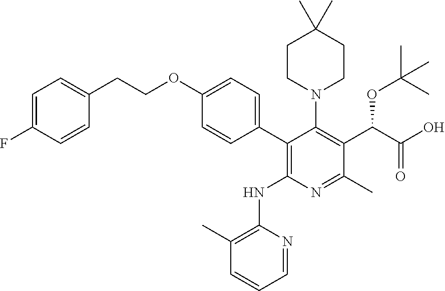 [SMILES: Cc1cccnc1Nc1nc(C)c([C@H](OC(C)(C)C)C(=O)O)c(N2CCC(C)(C)CC2)c1-c1ccc(OCCc2ccc(F)cc2)cc1]

### (S)-2-(tert-Butoxy)-2-(4-(4, 4-dimethylpiperidin-1-yl)-5-(4-(4-fluorophenethoxy)phenyl)-2-methyl-6-((3-methylpyridin-2-yl)amino)pyridin-3-yl)acetic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.04 g, 0.066 mmol), 2-bromo-3-methylpyridine (0.014 g, 0.079 mmol), Xantphos (2.292 mg, 3.96.mol), tris(dibenzylideneacetone)dipalladium(0) (1.209 mg, 1.321 μmol), and cesium carbonate (0.030 g, 0.092 mmol) in dioxane (1.1 mL) was degassed and heated at 100° C. for 5 h. The reaction mixture was cooled, filtered through a pad of celite, and concentrated in vacuo. The residue was taken up in ethanol (1 mL) and 5 M NaOH (0.132 mL, 0.660 mmol) was added. The mixture was heated at 100° C. for 1 h, cooled to ambient temperature, and filtered. The crude mixture was purified via preparative HPLC to afford the desired product (17.0 mg, 39%). ¹H NMR (500 MHz, DMSO-d₆) δ 7.97-7.91 (m, 1H), 7.43-7.27 (m, 4H), 7.16-7.08 (m, 3H), 7.04-6.96 (m, 2H), 6.85 (br dd, J=6.1, 5.3 Hz, 1H), 5.79 (br s, 1H), 4.26-4.13 (m, 2H), 3.02 (t, J=6.8 Hz, 2H), 2.78 (s, 1H), 2.55 (s, 1H), 2.35-2.28 (m, 3H), 2.21 (br d, J=11.7 Hz, 1H), 2.01 (s, 4H), 1.59-1.47 (m, 1H), 1.38-1.28 (m, 1H), 1.24-1.09 (m, 10H), 1.03 (br d, J=14.3 Hz, 1H), 0.90-0.80 (m, 3H), 0.67-0.57 (m, 3H). LCMS (M+1)=655.11.

### Example 66

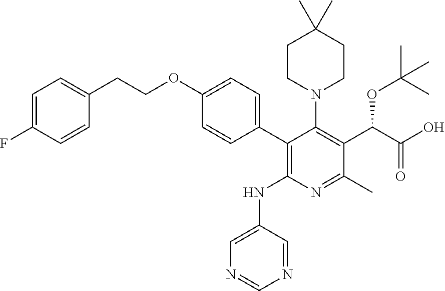 [SMILES: Cc1nc(Nc2cncnc2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-(pyrimidin-5-ylamino)pyridin-3-yl)acetic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.03 g, 0.050 mmol), 5-bromopyrimidine (0.011 g, 0.069 mmol, Xantphos (1.719 mg, 2.97 μmol), tris(dibenzylideneacetone)dipalladium(0) (0.907 mg, 0.990 μmol) and cesium carbonate (0.023 g, 0.069 mmol) in dioxane (0.495 mL) was degassed and heated at 100° C. for 3 h. The reaction mixture was cooled, filtered through a pad of celite, and concentrated in vacuo. The residue was taken up in methanol (1 mL) and 5 M NaOH (0.132 mL, 0.660 mmol) was added. The mixture was heated at 70° C. for 18 h, cooled to ambient temperature, and filtered. The crude mixture was purified via preparative HPLC to afford the desired product (19.5 mg, 59%). ¹H NMR (500 MHz, DMSO-d₆) δ 8.98 (s, 1H), 8.97 (br. s., 1H), 8.63 (s, 1H), 7.38 (dd, J=8.6, 5.7 Hz, 2H), 7.30 (d, J=8.1 Hz, 1H), 7.18-7.01 (m, 6H), 5.72 (br. s., 1H), 4.33-4.15 (m, 2H), 3.49 (br. s., 1H), 3.37 (br. s., 1H), 3.07 (t, J=6.6 Hz, 2H), 2.80 (t, J=11.7 Hz, 1H), 2.41 (s, 3H), 2.18 (d, J=9.2 Hz, 1H), 1.89-1.81 (m, 1H), 1.50 (d, J=11.0 Hz, 1H), 1.30 (d, J=8.4 Hz, 1H), 1.14 (s, 9H), 1.01 (d, J=11.4 Hz, 1H), 0.85 (s, 3H), 0.62 (s, 3H). LCMS (M+1)=642.1.

### Example 67

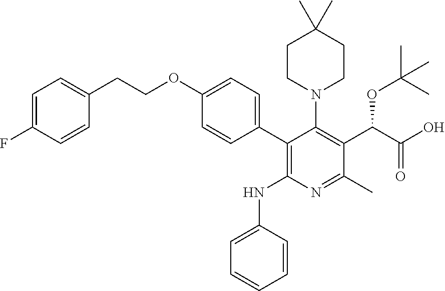 [SMILES: Cc1nc(Nc2ccccc2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-(phenylamino)pyridin-3-yl)acetic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.023 g, 0.038 mmol), bromobenzene (8.35 mg, 0.053 mmol), Xantphos (1.318 mg, 2.278 μmol), tris(dibenzylideneacetone)dipalladium(0) (0.695 mg, 0.759 μmol), and cesium carbonate (0.017 g, 0.053 mmol) in dioxane (0.380 mL) was degassed and heated at 100° C. for 18 h. The reaction mixture was cooled, filtered through a pad of celite, and concentrated in vacuo. The residue was taken up in methanol (1 mL) and 5 M NaOH (0.132 mL, 0.660 mmol) was added. The mixture was heated at 70° C. for 18 h, cooled to ambient temperature, and filtered. The crude mixture was purified via preparative HPLC to afford the desired product (10.0 mg, 41%). ¹H NMR (500 MHz, DMSO-d₆) δ 7.51-7.02 (m, 13H), 6.92 (br. s., 1H), 5.74 (br. s., 1H), 4.41-4.16 (m, 2H), 3.08 (t, J=6.4 Hz, 2H), 2.43 (s, 3H), 1.32-1.22 (m, 3H), 1.16 (s, 9H), 0.92-0.60 (m, 6H)[note: piperidine protons are very broad and poorly resolved]. LCMS (M+1)=640.1.

### Example 68

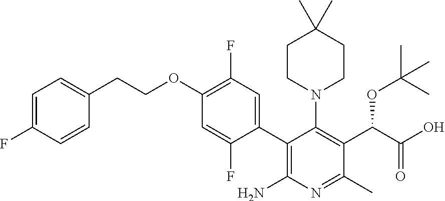 [SMILES: Cc1nc(N)c(-c2cc(F)c(OCCc3ccc(F)cc3)cc2F)c(N2CCC(C)(C)CC2)c1[C@H](OC(C)(C)C)C(=O)O]

### (2S)-2-(6-Amino-5-(2, 5-difluoro-4-(4-fluorophenethoxy)phenyl)-4-(4, 4-dimethylpiperidin-1-yl)-2-methylpyridin-3-yl)-2-(tert-butoxy)acetic acid

A mixture of (S)-isopropyl 2-(6-amino-5-bromo-4-(4,4-dimethylpiperidin-1-yl)-2-methylpyridin-3-yl)-2-(tert-butoxy)acetate (0.036 g, 0.077 mmol), 2-(2,5-difluoro-4-(4-fluorophenethoxy)phenyl)-6-methyl-1,3,6,2-dioxazaborocane-4,8-dione (0.047 g, 0.115 mmol), 2-dicyclohexylphosphino-2′,6′-dimethoxybiphenyl (9.42 mg, 0.023 mmol), palladium(II) acetate (2.58 mg, 0.011 mmol), and 2 M K₃PO₄ (0.459 mL, 0.918 mmol) in dioxane (1.53 mL) was purged with nitrogen and was heated at 80° C. for 2 h. The reaction mixture was diluted with ethyl acetate, washed with brine, dried (Na₂SO₄), and concentrated in vacuo. The residue was taken up in methanol (1 mL) and 10 N NaOH (0.1 mL, 1.0 mmol). The reaction was then heated to 70° C. for 6 h, cooled to ambient temperature, and filtered. The crude mixture was purified via preparative HPLC to afford the desired product (4.4 mg, 9%). ¹H NMR (500 MHz, DMSO-d6) δ 7.38 (dd, J=8.4, 5.5 Hz, 2H), 7.27 (dd, J=10.6, 7.3 Hz, 1H), 7.15 (t, J=9.0 Hz, 2H), 7.09 (dd, J=11.2, 7.2 Hz, 1H), 5.69 (br. s., 1H), 5.19 (s, 2H), 4.42-4.34 (m, 1H), 4.31-4.22 (m, 1H), 3.08 (t, J=6.6 Hz, 2H), 2.73 (t, J=12.8 Hz, 1H), 2.32-2.30 (m, 1H), 2.29 (s, 3H), 2.06 (t, J=11.9 Hz, 1H), 1.48 (br. s., 1H), 1.39-1.30 (m, 1H), 1.20 (br. s., 1H), 1.13 (s, 9H), 1.07 (br. s., 1H), 0.87 (s, 3H), 0.63 (s, 3H) [notes: piperidine protons poorly resolved and not all visible, may be rotamers present]. LCMS (M+1)=600.1.

### Example 69

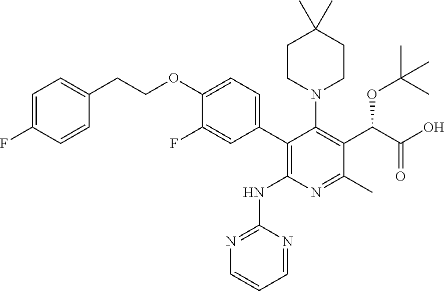 [SMILES: Cc1nc(Nc2ncccn2)c(-c2ccc(OCCc3ccc(F)cc3)c(F)c2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(3-fluoro-4-(4-fluorophenethoxy)phenyl)-2-methyl-6-(pyrimidin-2-ylamino)pyridin-3-yl)acetic acid

A mixture of (S)-isopropyl 2-(6-amino-4-(4,4-dimethylpiperidin-1-yl)-5-(3-fluoro-4-(4-fluorophenethoxy)phenyl)-2-methylpyridin-3-yl)-2-(tert-butoxy)acetate (0.031 g, 0.051 mmol), 2-bromopyrimidine (11 mg, 0.072 mmol), Xantphos (1.8 mg, 3 μmol), tris(dibenzylideneacetone)dipalladium(0) (1 mg, 1.0 μmol), and cesium carbonate (0.023 g, 0.072 mmol) in dioxane was degassed and heated at 100° C. for 3 h. The reaction mixture was cooled, filtered through a pad of celite, and concentrated in vacuo. The residue was taken up in methanol (1 mL) and 10 N NaOH (0.1 mL) was added. The mixture was heated at 70° C. for 18 h, cooled to ambient temperature, and filtered. The crude mixture was purified via preparative HPLC to afford the desired product (21.4 mg, 81%). LCMS (M+1)=660.3.

### Example 70 Example 71

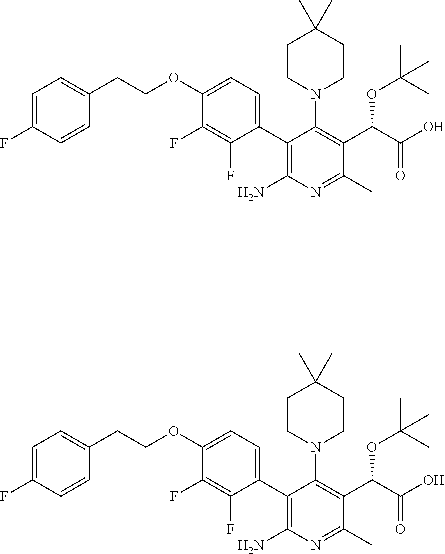 [SMILES: Cc1nc(N)c(-c2ccc(OCCc3ccc(F)cc3)c(F)c2F)c(N2CCC(C)(C)CC2)c1[C@H](OC(C)(C)C)C(=O)O ; Cc1nc(N)c(-c2ccc(OCCc3ccc(F)cc3)c(F)c2F)c(N2CCC(C)(C)CC2)c1[C@H](OC(C)(C)C)C(=O)O]

### (2S)-2-(6-Amino-5-(2, 3-difluoro-4-(4-fluorophenethoxy)phenyl)-4-(4, 4-dimethylpiperidin-1-yl)-2-methylpyridin-3-yl)-2-(tert-butoxy)acetic acid and atropisomer

A solution of (S)-isopropyl 2-(6-amino-5-bromo-4-(4,4-dimethylpiperidin-1-yl)-2-methylpyridin-3-yl)-2-(tert-butoxy)acetate (0.036 g, 0.077 mmol, 1 equiv), 2-(2,3-difluoro-4-(4-fluorophenethoxy)phenyl)-6-methyl-1,3,6,2-dioxazaborocane-4,8-dione (0.047 g, 0.115 mmol, 1.5 equiv), Pd(OAc)₂ (0.003 g, 0.12 mmol, 0.15 equiv), and SPhos (0.009 g, 0.23 mmol, 0.3 equiv) in degassed dioxane (1.5 mL) and 2 M K₃PO₄ (0.5 mL) was heated at 80° C. for 2 h. After cooling to ambient temperature, the reaction mixture was diluted with ethyl acetate, washed with brine, dried (Na₂SO₄), and concentrated in vacuo. The residue was taken up in methanol (1 mL) and 10 N NaOH (0.1 mL, 1.0 mmol). The reaction was then heated to 70° C. for 18 h, cooled to ambient temperature, and filtered. The crude mixture was purified via preparative HPLC to afford the product (3.6 mg, 8%) and its atropisomer (5.5 mg, 12%). 1st eluting isomer: ¹H NMR (500 MHz, DMSO-d₆) δ 7.42-7.35 (m, 2H), 7.26-7.13 (m, 5H), 7.03 (d, J=8.1 Hz, 1H), 5.43 (br. s., 1H), 4.50-4.22 (m, 2H), 3.15-2.99 (m, 2H), 2.45 (s, 3H), 1.26 (br. s., 4H), 1.16 (s, 9H), 0.78 (br. s., 6H) [note: piperidine protons are broad, poorly resolved, and not all visible; appears to be two rotamers]; LCMS (M+1)=600.1. 2^{nd} eluting isomer: ¹H NMR (500 MHz, DMSO-d6) δ 7.38 (dd, J=8.3, 5.7 Hz, 3H), 7.25-7.13 (m, 4H), 7.01 (t, J=7.7 Hz, 1H), 5.45 (br. s., 1H), 4.47-4.26 (m, 2H), 3.09 (t, J=6.6 Hz, 2H), 2.43 (s, 3H), 1.47 (br. s., 1H), 1.27 (br. s., 3H), 1.14 (s, 9H), 0.77 (br. s., 6H) [note: piperidine protons are broad, poorly resolved, and not all visible; appears to be two rotamers]; LCMS (M+1)=600.3.

### Example 72

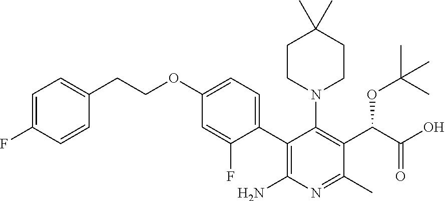 [SMILES: Cc1nc(N)c(-c2ccc(OCCc3ccc(F)cc3)cc2F)c(N2CCC(C)(C)CC2)c1[C@H](OC(C)(C)C)C(=O)O]

### (2S)-2-(6-Amino-4-(4, 4-dimethylpiperidin-1-yl)-5-(2-fluoro-4-(4-fluorophenethoxy)phenyl)-2-methylpyridin-3-yl)-2-(tert-butoxy)acetic acid

A solution of (S)-isopropyl 2-(6-amino-5-bromo-4-(4,4-dimethylpiperidin-1-yl)-2-methylpyridin-3-yl)-2-(tert-butoxy)acetate (0.042 g, 0.089 mmol, 1 equiv), 2-(2-fluoro-4-(4-fluorophenethoxy)phenyl)-6-methyl-1,3,6,2-dioxazaborocane-4,8-dione (0.052 g, 0.134 mmol, 1.5 equiv), Pd(OAc)₂ (0.003 g, 0.13 mmol, 0.15 equiv), and SPhos (0.011 g, 0.027 mmol, 0.3 equiv) in degassed dioxane (1.8 mL) and 2 M K₃PO₄ (0.5 mL) was heated at 80° C. for 2 h. After cooling to ambient temperature, the reaction mixture was diluted with ethyl acetate, washed with brine, dried (Na₂SO₄), and concentrated in vacuo. The residue was taken up in methanol (1 mL) and 10 N NaOH (0.1 mL). The reaction was then heated to 70° C. for 18 h, cooled to ambient temperature, and filtered. The crude mixture was purified via preparative HPLC to afford the product (20.2 mg, 37%). ¹H NMR (500 MHz, DMSO-d₆) δ 7.41-7.34 (m, 3H), 7.18-7.12 (m, 3H), 7.08 (t, J=8.6 Hz, 1H), 6.92 (br s, 2H), 5.68 (br s, 1H), 5.01 (s, 1H), 4.30-4.16 (m, 2H), 3.05 (t, J=6.4 Hz, 2H), 2.76-2.66 (m, 1H), 2.28-2.21 (m, 1H), 1.51-1.41 (m, 1H), 1.31 (br d, J=9.2 Hz, 1H), 1.19-1.15 (m, 1H), 1.13 (s, 9H), 1.09-1.00 (m, 2H), 0.85 (s, 3H), 0.60 (s, 3H) [note: piperidine protons are poorly resolved and not all visible; appears to be mixture of two rotamers]; LCMS (M+1)=582.3.

### Example 73

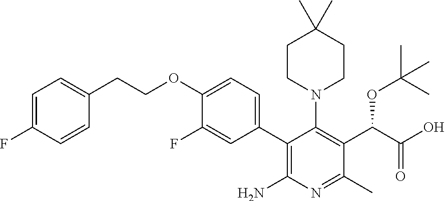 [SMILES: Cc1nc(N)c(-c2ccc(OCCc3ccc(F)cc3)c(F)c2)c(N2CCC(C)(C)CC2)c1[C@H](OC(C)(C)C)C(=O)O]

### (S)-2-(6-Amino-4-(4, 4-dimethylpiperidin-1-yl)-5-(3-fluoro-4-(4-fluorophenethoxy)phenyl)-2-methylpyridin-3-yl)-2-(tert-butoxy)acetic acid

A mixture of (S)-isopropyl 2-(6-amino-5-bromo-4-(4,4-dimethylpiperidin-1-yl)-2-methylpyridin-3-yl)-2-(tert-butoxy)acetate (0.033 g, 0.07 mmol), 2-(3-fluoro-4-(4-fluorophenethoxy)phenyl)-6-methyl-1,3,6,2-dioxazaborocane-4,8-dione (0.041 g, 0.105 mmol), 2-dicyclohexylphosphino-2′,6′-dimethoxybiphenyl (8.64 mg, 0.021 mmol), palladium(II) acetate (2.362 mg, 10.52 μmol), and 2 M K₃PO₄ (0.421 mL, 0.842 mmol) in dioxane (1.40 mL) was purged with nitrogen. The reaction was heated at 80° C. for 2 h. The reaction mixture was diluted with ethyl acetate, washed with brine, dried (Na₂SO₄), and concentrated in vacuo. The residue was taken up in methanol (1 mL) and 10 N NaOH (0.1 mL, 1.0 mmol), heated to 70° C., cooled to ambient temperature, and filtered. The crude mixture was purified via preparative HPLC to afford the desired product (13.2 mg, 32%). ¹H NMR (500 MHz, DMSO-d₆) δ 7.42-7.25 (m, 7H), 7.07-6.96 (m, 2H), 6.89 (br d, J=7.7 Hz, 1H), 7.45-6.84 (m, 7H), 5.70 (br s, 1H), 5.03 (br d, J=18.7 Hz, 2H), 4.44-4.19 (m, 2H), 3.53-3.19 (m, 1H), 3.14-3.01 (m, 2H), 2.85-2.70 (m, 1H), 2.28 (s, 3H), 2.25-2.17 (m, 1H), 2.01-1.83 (m, 1H), 1.53-1.40 (m, 1H), 1.35-1.25 (m, 1H), 1.23-1.15 (m, 1H), 1.13 (s, 9H), 1.10-0.96 (m, 1H), 0.85 (br s, 3H), 0.62 (br d, J=4.8 Hz, 3H) [note: appears to be two rotamers]. LCMS (M+1)=582.3.

### Example 74

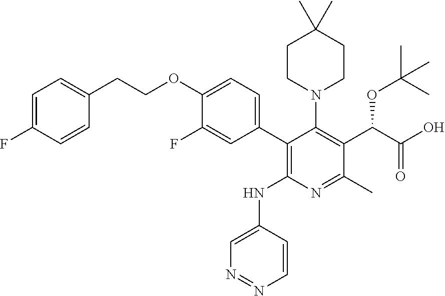 [SMILES: Cc1nc(Nc2ccnnc2)c(-c2ccc(OCCc3ccc(F)cc3)c(F)c2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(3-fluoro-4-(4-fluorophenethoxy)phenyl)-2-methyl-6-(pyridazin-4-ylamino)pyridin-3-yl)acetic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.02 g, 0.043 mmol), 4-bromopyridazine, hydrobromide (0.014 mg, 0.060 mmol), Xantphos (3.73 mg, 6.44 μmol), tris(dibenzylideneacetone)dipalladium(0) (1.965 mg, 2.146 μmol), and sodium tert-butoxide (12 mg, 0.129 mmol) in dioxane (0.858 mL) was degassed and heated at 100° C. for 2 h. The reaction mixture was cooled, filtered through a pad of celite, and concentrated in vacuo. The residue was taken up in methanol (1 mL) and 10 N NaOH (0.1 mL, 01.0 mmol) was added. The mixture was heated at 70° C. for 18 h, cooled to ambient temperature and filtered. The crude mixture was purified via preparative HPLC to afford the desired product (14.0 mg, 51%). ¹H NMR (500 MHz, DMSO-d6) δ 9.28-9.07 (m, 1H), 8.75 (br d, J=5.9 Hz, 1H), 7.94 (dd, J=5.3, 3.1 Hz, 1H), 7.53 (s, 1H), 7.40 (br dd, J=8.3, 5.7 Hz, 2H), 7.28 (br d, J=8.4 Hz, 1H), 7.20-7.11 (m, 4H), 7.07 (br s, 1H), 5.78 (br s, 1H), 4.40-4.15 (m, 2H), 3.37-3.23 (m, 1H), 3.14-3.02 (m, 2H), 2.87-2.76 (m, 1H), 2.48 (s, 3H), 2.23-2.08 (m, 1H), 1.91-1.76 (m, 1H), 1.58-1.43 (m, 1H), 1.37-1.26 (m, 1H), 1.23-1.18 (m, 1H), 1.16 (s, 9H), 1.02 (br d, J=14.7 Hz, 1H), 0.86 (br s, 3H), 0.63 (br s, 3H). LCMS (M+1)=642.3.

### Example 75

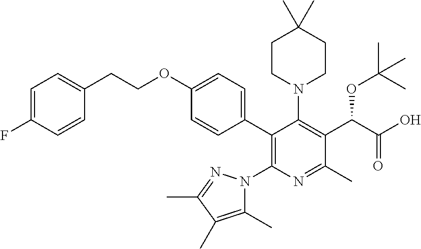 [SMILES: Cc1nn(-c2nc(C)c([C@H](OC(C)(C)C)C(=O)O)c(N3CCC(C)(C)CC3)c2-c2ccc(OCCc3ccc(F)cc3)cc2)c(C)c1C]

### (S)-2-(tert-Butoxy)-2-(4-(4, 4-dimethylpiperidin-1-yl)-5-(4-(4-fluorophenethoxy)phenyl)-2-methyl-6-(3,4, 5-trimethyl-H-pyrazol-1-yl)pyridin-3-yl)acetic acid

3-Methylpentane-2,4-dione (8.09 mg, 0.071 mmol) and 1 M HCl (0.039 ml, 0.039 mmol) were added to a solution of (S)-isopropyl 2-(tert-butoxy)-2-(4-(4,4-dimethylpiperidin-1-yl)-5-(4-(4-fluorophenethoxy)phenyl)-6-hydrazinyl-2-methylpyridin-3-yl)acetate (0.022 g, 0.035 mmol) in ethanol (0.5 mL) and heated to 80° C. for 2 h. 10 N NaOH (0.1 mL, 1.0 mmol) was then added and heating was continued at 80° C. for 2 h. After cooling to ambient temperature, the reaction was filtered. The crude mixture was purified via preparative HPLC to afford the desired product (12.3 mg, 52%). ¹H NMR (500 MHz, DMSO-d₆) δ 7.33 (dd, J=8.6, 5.7 Hz, 2H), 7.12 (br d, J=18.0 Hz, 2H), 7.06 (br d, J=8.8 Hz, 1H), 6.93 (brt, J=10.1 Hz, 2H), 6.74 (br d, J=7.0 Hz, 1H), 5.76 (br s, 1H), 4.20-4.07 (m, 2H), 3.58-3.46 (m, 1H), 2.99 (t, J=6.6 Hz, 2H), 2.94-2.82 (m, 1H), 2.47 (s, 3H), 2.23-2.10 (m, 1H), 1.96 (s, 3H), 1.89-1.82 (m, 1H), 1.76 (s, 3H), 1.73 (s, 3H), 1.62-1.51 (m, 1H), 1.39-1.20 (m, 2H), 1.14 (s, 9H), 1.09-0.98 (m, 1H), 0.87 (br s, 3H), 0.65 (br s, 3H). LCMS (M+1)=657.

### Example 76

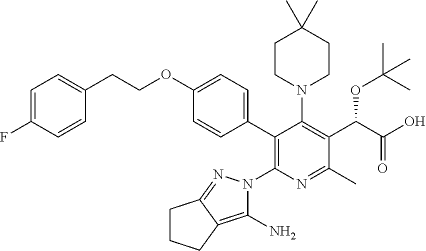 [SMILES: Cc1nc(-n2nc3c(c2N)CCC3)c(-c2ccc(OCCc3ccc(F)cc3)cc2)c(N2CCC(C)(C)CC2)c1[C@H](OC(C)(C)C)C(=O)O]

### (S)-2-(6-(3-Amino-5, 6-dihydrocyclopenta[c]pyrazol-2(4H)-yl)-4-(4, 4-dimethylpiperidin-1-yl)-5-(4-(4-fluorophenethoxy)phenyl)-2-methylpyridin-3-yl)-2-(tert-butoxy)acetic acid

2-Oxocyclopentanecarbonitrile (0.011 g, 0.103 mmol) and 1 M HCl (0.057 ml, 0.057 mmol) were added to a solution of (S)-isopropyl 2-(tert-butoxy)-2-(4-(4,4-dimethylpiperidin-1-yl)-5-(4-(4-fluorophenethoxy)phenyl)-6-hydrazinyl-2-methylpyridin-3-yl)acetate (0.032 g, 0.052 mmol) in ethanol (0.5 mL) and heated to 80° C. for 2 h. 10 N NaOH (0.1 mL, 1.0 mmol) was added and heating was continued at 80° C. for 2 h. The reaction was then cooled to ambient temperature and filtered. The crude mixture was purified via preparative HPLC to afford the desired product (10.8 mg, 31%). ¹H NMR (500 MHz, DMSO-d₆) δ 7.35 (dd, J=8.4, 5.9 Hz, 2H), 7.22-7.16 (m, 1H), 7.13 (brt, J=9.0 Hz, 3H), 7.10-7.05 (m, 1H), 6.96-6.88 (m, 1H), 6.85-6.77 (m, 1H), 5.77 (br s, 1H), 4.24-4.12 (m, 2H), 3.55-3.42 (m, 1H), 3.01 (t, J=6.6 Hz, 2H), 2.91-2.79 (m, 1H), 2.48 (s, 3H), 2.35-2.28 (m, 4H), 2.20-2.09 (m, 3H), 1.57-1.49 (m, 1H), 1.34-1.26 (m, 1H), 1.25-1.18 (m, 1H), 1.15 (s, 9H), 1.08-0.98 (m, 1H), 0.87 (br s, 3H), 0.63 (br s, 3H). LCMS (M+1)=670.4.

### Example 77

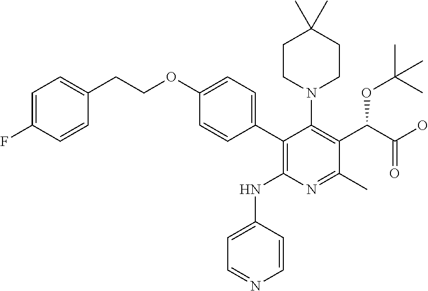 [SMILES: Cc1nc(Nc2ccncc2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-(pyridin-4-ylamino)pyridin-3-yl)acetic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.025 g, 0.041 mmol), 4-bromopyridine hydrochloride (8.03 mg, 0.041 mmol), Xantphos (1.433 mg, 2.476 μmol), tris(dibenzylideneacetone)dipalladium(0) (0.756 mg, 0.825 μmol), and sodium tert-butoxide (9.52 mg, 0.099 mmol) in dioxane (1 mL) was degassed and heated at 100° C. for 5 h. The reaction mixture was cooled to ambient temperature, filtered through a pad of celite, and concentrated in vacuo. The residue was taken up in ethanol (1 mL) and 5 M NaOH (0.083 mL, 0.413 mmol) was added. The mixture was heated at 100° C. for 1 h, cooled to ambient temperature, and filtered. The crude mixture was purified via preparative HPLC to afford the desired product (6.9 mg, 26%). ¹H NMR (500 MHz, DMSO-d₆) δ 8.19 (br d, J=5.9 Hz, 2H), 7.48 (d, J=5.9 Hz, 2H), 7.39 (dd, J=8.3, 5.7 Hz, 2H), 7.32-7.27 (m, 1H), 7.20-7.03 (m, 6H), 5.79-5.73 (m, 1H), 4.33-4.21 (m, 2H), 3.11-3.04 (m, 2H), 2.85-2.78 (m, 1H), 2.47 (s, 3H), 2.19 (br d, J=11.7 Hz, 1H), 1.90-1.90 (m, 1H), 1.90-1.81 (m, 1H), 1.55-1.46 (m, 1H), 1.36-1.27 (m, 1H), 1.22-1.13 (m, 10H), 1.03 (br d, J=12.1 Hz, 1H), 0.86 (s, 3H), 0.62 (s, 3H). LCMS (M+1)=641.3.

### Example 78

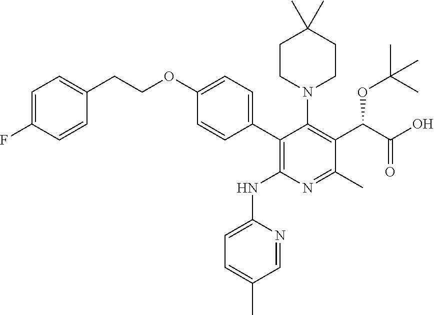 [SMILES: Cc1ccc(Nc2nc(C)c([C@H](OC(C)(C)C)C(=O)O)c(N3CCC(C)(C)CC3)c2-c2ccc(OCCc3ccc(F)cc3)cc2)nc1]

### (S)-2-(tert-Butoxy)-2-(4-(4, 4-dimethylpiperidin-1-yl)-5-(4-(4-fluorophenethoxy)phenyl)-2-methyl-6-((5-methylpyridin-2-yl)amino)pyridin-3-yl)acetic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.025 g, 0.041 mmol), 2-bromo-5-methylpyridine (8.52 mg, 0.050 mmol), Xantphos (1.433 mg, 2.476 μmol), tris(dibenzylideneacetone)dipalladium(0) (0.756 mg, 0.825 μmol), and cesium carbonate (0.019 g, 0.058 mmol) in dioxane (1 mL) was degassed and heated at 100° C. for 5 h. The reaction mixture was cooled to ambient temperature, filtered through a pad of celite, and concentrated in vacuo. The residue was taken up in ethanol (1 mL) and 5 M NaOH (0.083 mL, 0.413 mmol) was added. The mixture was heated at 100° C. for 1 h, cooled to ambient temperature, and filtered. The crude mixture was purified via preparative HPLC to afford the desired product (11.4 mg, 42%). ¹H NMR (500 MHz, DMSO-d₆) δ 8.42-8.38 (m, 1H), 7.85 (s, 1H), 7.52 (br d, J=8.4 Hz, 1H), 7.44-7.32 (m, 3H), 7.22-7.12 (m, 5H), 6.76 (s, 1H), 5.75 (br s, 1H), 4.35-4.23 (m, 2H), 3.09 (t, J=6.6 Hz, 2H), 2.79 (brt, J=11.7 Hz, 1H), 2.47 (s, 3H), 2.25 (br d, J=12.5 Hz, 1H), 2.18 (s, 3H), 1.99-1.88 (m, 3H), 1.58-1.46 (m, 1H), 1.37-1.27 (m, 1H), 1.22-1.12 (m, 10H), 1.09-1.00 (m, 1H), 0.86 (s, 3H), 0.62 (s, 3H). LCMS (M+1)=655.3.

### Example 79

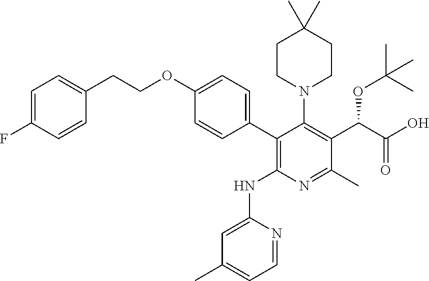 [SMILES: Cc1ccnc(Nc2nc(C)c([C@H](OC(C)(C)C)C(=O)O)c(N3CCC(C)(C)CC3)c2-c2ccc(OCCc3ccc(F)cc3)cc2)c1]

### (S)-2-(tert-Butoxy)-2-(4-(4, 4-dimethylpiperidin-1-yl)-5-(4-(4-fluorophenethoxy)phenyl)-2-methyl-6-((4-methylpyridin-2-yl)amino)pyridin-3-yl)acetic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.025 g, 0.041 mmol), 2-bromo-4-methylpyridine (8.52 mg, 0.050 mmol), Xantphos (1.433 mg, 2.476 mol), tris(dibenzylideneacetone)dipalladium(0) (0.756 mg, 0.825 μmol), and cesium carbonate (0.019 g, 0.058 mmol) in dioxane (1 mL) was degassed and heated at 100° C. for 5 h. The reaction mixture was cooled to ambient temperature, filtered through a pad of celite, and concentrated in vacuo. The residue was taken up in ethanol (1 mL) and 5 M NaOH (0.083 mL, 0.413 mmol) was added. The mixture was heated at 100° C. for 1 h, cooled to ambient temperature, and filtered. The crude mixture was purified via preparative HPLC to afford the desired product (9.8 mg, 36%). ¹H NMR (500 MHz, DMSO-d₆) δ 8.36-8.32 (m, 1H), 7.90 (d, J=5.1 Hz, 1H), 7.43-7.32 (m, 3H), 7.22-7.11 (m, 5H), 6.80 (s, 1H), 6.71 (br d, J=4.8 Hz, 1H), 5.83-5.74 (m, 1H), 4.34-4.23 (m, 2H), 3.09 (br t, J=6.8 Hz, 2H), 2.84 (br d, J=2.9 Hz, 1H), 2.50-2.46 (m, 3H), 2.33-2.22 (m, 4H), 2.00-1.89 (m, 2H), 1.57-1.47 (m, 1H), 1.37-1.26 (m, 1H), 1.21-1.13 (m, 10H), 1.04 (br d, J=13.2 Hz, 1H), 0.86 (s, 3H), 0.62 (s, 3H). LCMS (M+1)=655.33.

### Example 80

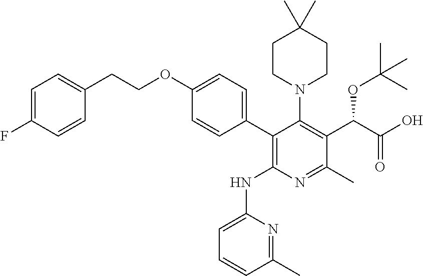 [SMILES: Cc1cccc(Nc2nc(C)c([C@H](OC(C)(C)C)C(=O)O)c(N3CCC(C)(C)CC3)c2-c2ccc(OCCc3ccc(F)cc3)cc2)n1]

### (S)-2-(tert-Butoxy)-2-(4-(4, 4-dimethylpiperidin-1-yl)-5-(4-(4-fluorophenethoxy)phenyl)-2-methyl-6-((6-methylpyridin-2-yl)amino)pyridin-3-yl)acetic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.025 g, 0.041 mmol), 2-bromo-6-methylpyridine (8.52 mg, 0.050 mmol), Xantphos (1.433 mg, 2.476 μmol), tris(dibenzylideneacetone)dipalladium(0) (0.756 mg, 0.825 μmol), and cesium carbonate (0.019 g, 0.058 mmol) in dioxane (1 mL) was degassed and heated at 100° C. for 5 h. The reaction mixture was cooled, filtered through a pad of celite, and concentrated in vacuo. The residue was taken up in ethanol (1 mL) and 5 M NaOH (0.083 mL, 0.413 mmol) was added. The mixture was heated at 100° C. for 1 h, cooled to ambient temperature, and filtered. The crude mixture was purified via preparative HPLC to afford the desired product (13.9 mg, 51%). ¹H NMR (500 MHz, DMSO-d6) δ 8.34-8.28 (m, 1H), 7.61-7.54 (m, 1H), 7.43-7.31 (m, 3H), 7.21-7.10 (m, 5H), 6.81-6.71 (m, 1H), 5.76 (br s, 1H), 4.34-4.21 (m, 2H), 3.35-3.27 (m, 1H), 3.11-3.04 (m, 2H), 2.23 (s, 4H), 1.95-1.85 (m, 2H), 1.54-1.46 (m, 1H), 1.34-1.25 (m, 1H), 1.21-1.10 (m, 11H), 1.05-1.00 (m, 1H), 0.85 (s, 3H), 0.60 (s, 3H). LCMS (M+1)=655.28.

### Example 81

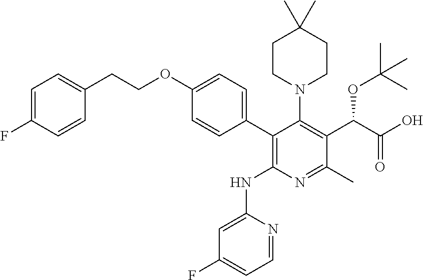 [SMILES: Cc1nc(Nc2cc(F)ccn2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-6-((4-fluoropyridin-2-yl)amino)-2-methylpyridin-3-yl)acetic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.025 g, 0.041 mmol), 2-bromo-4-fluoropyridine (8.72 mg, 0.050 mmol), Xantphos (1.433 mg, 2.476 mol), tris(dibenzylideneacetone)dipalladium(0) (0.756 mg, 0.825 μmol), and cesium carbonate (0.019 g, 0.058 mmol) in dioxane (1 mL) was degassed and heated at 100° C. for 5 h. The reaction mixture was cooled, filtered through a pad of celite, and concentrated. The residue was taken up in ethanol (1 mL) and 5 M NaOH (0.083 mL, 0.413 mmol) was added. The mixture was heated at 100° C. for 1 h, cooled to ambient temperature, and filtered. The crude mixture was purified via preparative HPLC to afford the desired product (5.7 mg, 20%) and its estimated purity by LCMS analysis was. ¹H NMR (500 MHz, DMSO-d₆) δ 8.36-8.29 (m, 1H), 8.07 (dd, J=9.2, 5.9 Hz, 1H), 7.44-7.33 (m, 3H), 7.21-7.11 (m, 5H), 7.05 (s, 1H), 6.82-6.76 (m, 1H), 5.76 (br s, 1H), 4.37-4.20 (m, 2H), 3.62-3.54 (m, 1H), 3.38-3.29 (m, 1H), 3.20-3.14 (m, 1H), 3.07 (brt, J=6.4 Hz, 2H), 2.79 (br t, J=11.9 Hz, 1H), 2.49 (s, 3H), 2.28-2.18 (m, 1H), 1.93 (br t, J=11.6 Hz, 1H), 1.55-1.45 (m, 1H), 1.37-1.26 (m, 1H), 1.23-1.10 (m, 10H), 1.08-1.00 (m, 1H), 0.89-0.81 (m, 3H), 0.61 (s, 3H). LCMS (M+1)=659.3.

### Example 82

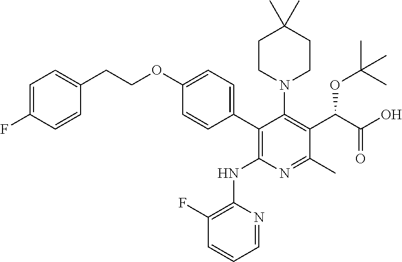 [SMILES: Cc1nc(Nc2ncccc2F)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-6-((3-fluoropyridin-2-yl)amino)-2-methylpyridin-3-yl)acetic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.025 g, 0.041 mmol), 2-bromo-3-fluoropyridine (8.72 mg, 0.050 mmol), Xantphos (1.433 mg, 2.476 mol), tris(dibenzylideneacetone)dipalladium(0) (0.756 mg, 0.825 μmol), and cesium carbonate (0.019 g, 0.058 mmol) in dioxane (1 mL) was degassed and heated at 100° C. for 5 h. The reaction mixture was cooled, filtered through a pad of celite, and concentrated in vacuo. The residue was taken up in ethanol (1 mL) and 5 M NaOH (0.083 mL, 0.413 mmol) was added. The mixture was heated at 100° C. for 1 h, cooled to ambient temperature, and filtered. The crude mixture was purified via preparative HPLC to afford the desired product (20.1 mg, 74%). ¹H NMR (500 MHz, DMSO-d₆) δ 7.93-7.88 (m, 1H), 7.47 (dd, J=10.3, 8.8 Hz, 1H), 7.38-7.26 (m, 3H), 7.16-7.07 (m, 3H), 7.04-6.96 (m, 2H), 6.92 (dt, J=8.1, 4.0 Hz, 1H), 5.76 (br s, 1H), 4.27-4.13 (m, 2H), 3.40-3.32 (m, 1H), 3.02 (brt, J=6.6 Hz, 2H), 2.86-2.78 (m, 1H), 2.55-2.53 (m, 1H), 2.31 (s, 3H), 2.19 (br d, J=8.8 Hz, 1H), 1.98-1.92 (m, 1H), 1.55-1.46 (m, 1H), 1.35-1.25 (m, 1H), 1.23-1.17 (m, 1H), 1.13 (s, 9H), 1.06-1.00 (m, 1H), 0.85 (s, 3H), 0.61 (s, 3H). LCMS (M+1)=659.26.

### Example 83

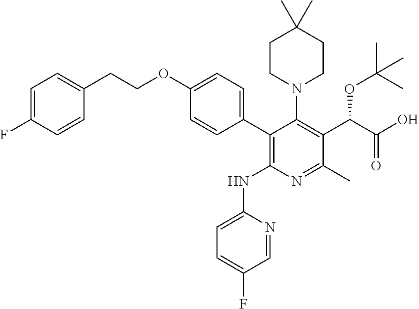 [SMILES: Cc1nc(Nc2ccc(F)cn2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-6-((5-fluoropyridin-2-yl)amino)-2-methylpyridin-3-yl)acetic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.025 g, 0.041 mmol), 2-bromo-5-fluoropyridine (8.72 mg, 0.050 mmol), Xantphos (1.433 mg, 2.476 μmol), tris(dibenzylideneacetone)dipalladium(0) (0.756 mg, 0.825 μmol), and cesium carbonate (0.019 g, 0.058 mmol) in dioxane (1 mL) was degassed and heated at 100° C. for 5 h. The reaction mixture was cooled to ambient temperature, filtered through a pad of celite, and concentrated in vacuo. The residue was taken up in ethanol (1 mL) and 5 M NaOH (0.083 mL, 0.413 mmol) was added. The mixture was heated at 100° C. for 1 h, cooled to ambient temperature, and filtered. The crude mixture was purified via preparative HPLC to afford the desired product (10.2 mg, 38%). ¹H NMR (500 MHz, DMSO-d₆) δ 8.55-8.47 (m, 1H), 8.06-8.01 (m, 1H), 7.70-7.61 (m, 1H), 7.44-7.31 (m, 3H), 7.22-7.09 (m, 5H), 6.87 (s, 1H), 5.83-5.73 (m, 1H), 4.32-4.22 (m, 2H), 3.30 (br d, J=11.4 Hz, 1H), 3.07 (brt, J=6.6 Hz, 2H), 2.83-2.75 (m, 1H), 2.47 (s, 3H), 2.27-2.20 (m, 1H), 1.98-1.89 (m, 1H), 1.54-1.45 (m, 1H), 1.36-1.25 (m, 1H), 1.22-1.09 (m, 10H), 1.03 (br d, J=11.4 Hz, 1H), 0.85 (s, 3H), 0.60 (s, 3H). LCMS (M+1)=659.31.

### Example 84

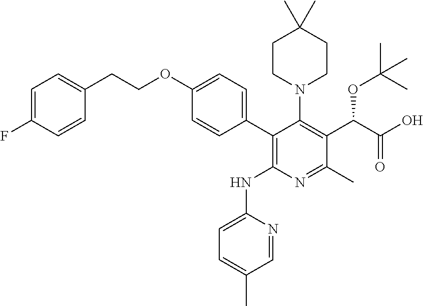 [SMILES: Cc1ccc(Nc2nc(C)c([C@H](OC(C)(C)C)C(=O)O)c(N3CCC(C)(C)CC3)c2-c2ccc(OCCc3ccc(F)cc3)cc2)nc1]

### (S)-2-(tert-Butoxy)-2-(4-(4, 4-dimethylpiperidin-1-yl)-5-(4-(4-fluorophenethoxy)phenyl)-2-methyl-6-((6-methylpyridin-3-yl)amino)pyridin-3-yl)acetic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.025 g, 0.041 mmol), 5-bromo-2-methylpyridine (8.52 mg, 0.050 mmol), Xantphos (1.433 mg, 2.476 μmol), tris(dibenzylideneacetone)dipalladium(0) (0.756 mg, 0.825 μmol), and cesium carbonate (0.019 g, 0.058 mmol) in dioxane (1 mL) was degassed and heated at 100° C. for 5 h. The reaction mixture was cooled to ambient temperature, filtered through a pad of celite, and concentrated. The residue was taken up in ethanol (1 mL) and 5 M NaOH (0.083 mL, 0.413 mmol) was added. The mixture was heated at 100° C. for 1 h, cooled to ambient temperature, and filtered. The crude mixture was purified via preparative HPLC to afford the desired product (6.5 mg, 24%). ¹H NMR (500 MHz, DMSO-d6) δ 8.52-8.46 (m, 1H), 7.84 (dd, J=8.3, 2.8 Hz, 1H), 7.38 (dd, J=8.3, 5.7 Hz, 2H), 7.33-7.27 (m, 1H), 7.19-7.03 (m, 6H), 6.61 (s, 1H), 5.76 (br s, 1H), 4.32-4.19 (m, 2H), 3.31-3.23 (m, 1H), 3.06 (br t, J=6.6 Hz, 2H), 2.83-2.75 (m, 1H), 2.36 (d, J=9.9 Hz, 6H), 2.23-2.16 (m, 1H), 1.93-1.83 (m, 1H), 1.54-1.44 (m, 1H), 1.30 (td, J=13.0, 4.4 Hz, 1H), 1.21-1.10 (m, 10H), 1.05-0.98 (m, 1H), 0.84 (s, 3H), 0.60 (s, 3H). LCMS (M+1)=655.33.

### Example 85

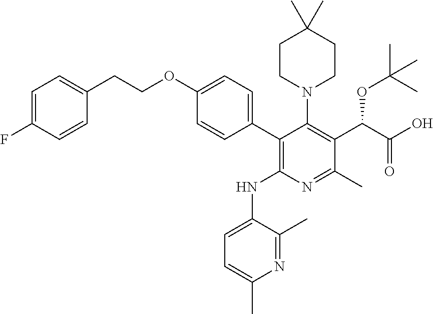 [SMILES: Cc1ccc(Nc2nc(C)c([C@H](OC(C)(C)C)C(=O)O)c(N3CCC(C)(C)CC3)c2-c2ccc(OCCc3ccc(F)cc3)cc2)c(C)n1]

### (S)-2-(tert-Butoxy)-2-(4-(4, 4-dimethylpiperidin-1-yl)-6-((2, 6-dimethylpyridin-3-yl)amino)-5-(4-(4-fluorophenethoxy)phenyl)-2-methylpyridin-3-yl)acetic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.025 g, 0.041 mmol), 3-bromo-2,6-dimethylpyridine (9.21 mg, 0.050 mmol, Xantphos (1.433 mg, 2.476 μmol), tris(dibenzylideneacetone)dipalladium(0) (0.756 mg, 0.825 μmol), and cesium carbonate (0.019 g, 0.058 mmol) in dioxane (1 mL) was degassed and heated at 100° C. for 5 h. The reaction mixture was cooled to ambient temperature, filtered through a pad of celite, and concentrated in vacuo. The residue was taken up in ethanol (1 mL) and 5 M NaOH (0.083 mL, 0.413 mmol) was added. The mixture was heated at 100° C. for 1 h, cooled to ambient temperature, and filtered. The crude mixture was purified via preparative HPLC to afford the desired product (10.5 mg, 38%). ¹H NMR (500 MHz, DMSO-d₆) δ 8.40-8.32 (m, 1H), 7.46-7.34 (m, 3H), 7.25-7.10 (m, 5H), 7.00 (d, J=8.1 Hz, 1H), 6.17-6.10 (m, 1H), 5.77 (br s, 1H), 4.34-4.20 (m, 2H), 3.07 (br t, J=6.8 Hz, 2H), 2.81 (brt, J=11.6 Hz, 1H), 2.35 (d, J=18.0 Hz, 7H), 2.04-1.92 (m, 4H), 1.56-1.45 (m, 1H), 1.36-1.27 (m, 1H), 1.23-1.10 (m, 10H), 1.08-1.01 (m, 1H), 0.86 (s, 3H), 0.62 (s, 3H). LCMS (M+1)=669.3.

### Example 86

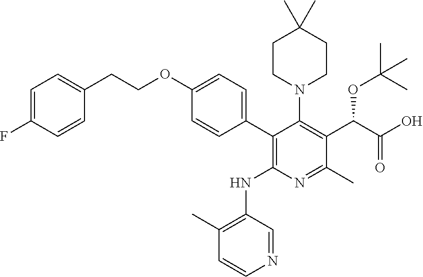 [SMILES: Cc1ccncc1Nc1nc(C)c([C@H](OC(C)(C)C)C(=O)O)c(N2CCC(C)(C)CC2)c1-c1ccc(OCCc2ccc(F)cc2)cc1]

### (S)-2-(tert-Butoxy)-2-(4-(4, 4-dimethylpiperidin-1-yl)-5-(4-(4-fluorophenethoxy)phenyl)-2-methyl-6-((4-methylpyridin-3-yl)amino)pyridin-3-yl)acetic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.025 g, 0.041 mmol), 3-bromo-4-methylpyridine (8.52 mg, 0.050 mmol), Xantphos (1.433 mg, 2.476 μmol), tris(dibenzylideneacetone)dipalladium(0) (0.756 mg, 0.825 μmol), and cesium carbonate (0.019 g, 0.058 mmol) in dioxane (1 mL) was degassed and heated at 100° C. for 5 h. The reaction mixture was cooled to ambient temperature, filtered through a pad of celite, and concentrated in vacuo. The residue was taken up in ethanol (1 mL) and 5 M NaOH (0.083 mL, 0.413 mmol) was added. The mixture was heated at 100° C. for 1 h, cooled to ambient temperature, and filtered. The crude mixture was purified via preparative HPLC to afford the desired product (7.4 mg, 26%). ¹H NMR (500 MHz, DMSO-d₆) δ 9.26-9.16 (m, 1H), 8.06 (d, J=4.8 Hz, 1H), 7.48-7.34 (m, 3H), 7.25-7.07 (m, 6H), 6.26 (s, 1H), 5.84-5.76 (m, 1H), 4.35-4.18 (m, 2H), 3.12-3.01 (m, 2H), 2.88-2.79 (m, 1H), 2.57 (br d, J=2.6 Hz, 2H), 2.37 (s, 3H), 2.31-2.23 (m, 1H), 1.87 (s, 3H), 1.57-1.46 (m, 1H), 1.37-1.28 (m, 1H), 1.25-1.12 (m, 11H), 1.10-1.02 (m, 1H), 0.86 (s, 3H), 0.65-0.59 (m, 3H). LCMS (M+1)=655.33.

### Example 87

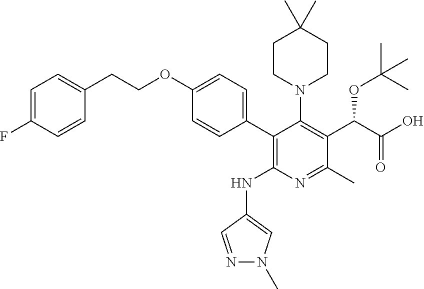 [SMILES: Cc1nc(Nc2cnn(C)c2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-((1-methyl-H-pyrazol-4-yl)amino)pyridin-3-yl)acetic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.036 g, 0.06 mmol), 4-bromo-1-methyl-1H-pyrazole (0.012 g, 0.072 mmol), BrettPhos (0.966 mg, 1.800 μmol), BrettPhos precatalyst (1.438 mg, 1.800 μmol), and sodium tert-butoxide (5.77 mg, 0.060 mmol) in dioxane (1 mL) heated at 110° C., heated for 23 h, cooled to ambient temperature, filtered through a pad of celite, and concentrated in vacuo. The residue was taken up in ethanol (1 mL) and 5 M NaOH (0.120 ml, 0.600 mmol) was added. The mixture was heated at 80° C. for 2 h, cooled to ambient temperature, and filtered. The crude mixture was purified via preparative HPLC to afford the desired product (3.4 mg, 9%). ¹H NMR (500 MHz, DMSO-d₆) δ 8.02-7.97 (m, 1H), 7.45-7.38 (m, 3H), 7.26-7.06 (m, 6H), 6.55 (s, 1H), 5.74 (br s, 1H), 4.32-4.21 (m, 2H), 3.75 (s, 3H), 3.08 (t, J=6.8 Hz, 2H), 2.77 (br t, J=12.1 Hz, 1H), 2.43 (s, 3H), 2.18 (br d, J=9.9 Hz, 1H), 1.82 (s, 1H), 1.55-1.45 (m, 1H), 1.36-1.26 (m, 1H), 1.20-1.12 (m, 10H), 1.05-0.98 (m, 1H), 0.85 (s, 3H), 0.61 (s, 3H). LCMS (M+1)=644.42.

### Example 88

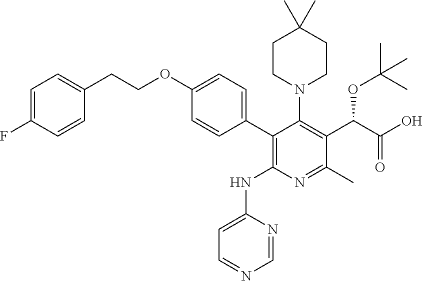 [SMILES: Cc1nc(Nc2ccncn2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-(pyrimidin-4-ylamino)pyridin-3-yl)acetic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.025 g, 0.041 mmol), 4-chloropyrimidine (5.67 mg, 0.050 mmol), Xantphos (1.433 mg, 2.476 μmol), tris(dibenzylideneacetone)dipalladium(0) (0.756 mg, 0.825 μmol), and cesium carbonate (0.019 g, 0.058 mmol) in dioxane (1 mL) was degassed and heated at 100° C. for 5 h. The reaction mixture was cooled to ambient temperature, filtered through a pad of celite, and concentrated in vacuo. The residue was taken up in ethanol (1 mL) and 5 M NaOH (0.083 mL, 0.413 mmol) was added. The mixture was heated at 100° C. for 1 h, cooled to ambient temperature, and filtered. The crude mixture was purified via preparative HPLC to afford the desired product (3.1 mg, 12%). LCMS (M+1)=642.28

### Example 89

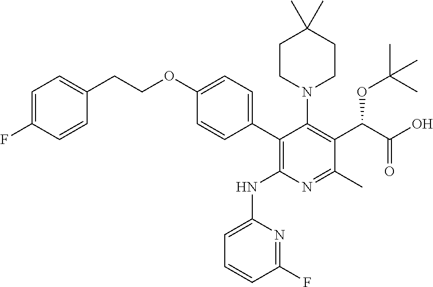 [SMILES: Cc1nc(Nc2cccc(F)n2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-6-((6-fluoropyridin-2-yl)amino)-2-methylpyridin-3-yl)acetic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.025 g, 0.041 mmol), 2-bromo-6-fluoropyridine (8.72 mg, 0.050 mmol), Xantphos (1.433 mg, 2.476 μmol), tris(dibenzylideneacetone)dipalladium(0) (0.756 mg, 0.825 μmol), and cesium carbonate (0.019 g, 0.058 mmol) in dioxane (1 mL) was degassed and heated at 100° C. for 5 h. The reaction mixture was cooled to ambient temperature, filtered through a pad of celite, and concentrated. The residue was taken up in ethanol (1 mL) and 5 M NaOH (0.083 mL, 0.413 mmol) was added. The mixture was heated at 100° C. for 1 h, cooled to ambient temperature, and filtered. The crude mixture was purified via preparative HPLC to afford the desired product (10.3 mg, 35%). LCMS (M+1)=659.31.

### Example 90

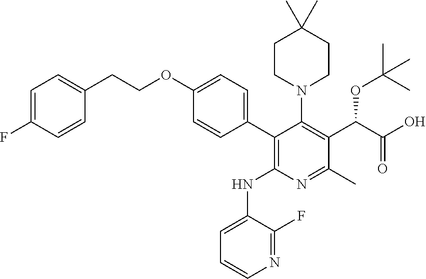 [SMILES: Cc1nc(Nc2cccnc2F)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-6-((2-fluoropyridin-3-yl)amino)-2-methylpyridin-3-yl)acetic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.025 g, 0.041 mmol), 3-bromo-2-fluoropyridine (8.72 mg, 0.050 mmol), Xantphos (1.433 mg, 2.476 mol), tris(dibenzylideneacetone)dipalladium(0) (0.756 mg, 0.825 μmol), and cesium carbonate (0.019 g, 0.058 mmol) in dioxane (1 mL) was degassed and heated at 100° C. for 5 h. The reaction mixture was cooled to ambient temperature, filtered through a plug of celite, and concentrated in vacuo. The residue was taken up in ethanol (1 mL) and 5 M NaOH (0.083 mL, 0.413 mmol) was added. The mixture was heated at 100° C. for 1 h, cooled to ambient temperature, and filtered. The crude mixture was purified via preparative HPLC to afford the desired product (9.7 mg, 36%). ¹H NMR (500 MHz, DMSO-d₆) δ 9.07-8.77 (m, 1H), 7.65 (br d, J=4.4 Hz, 1H), 7.49-6.99 (m, 9H), 6.52 (d, J=3.3 Hz, 1H), 5.69 (s, 1H), 4.37-4.15 (m, 2H), 3.12-3.03 (m, 2H), 2.86-2.74 (m, 1H), 2.55 (s, 3H), 2.45 (s, 3H), 2.31-2.21 (m, 1H), 1.56-1.47 (m, 1H), 1.38-1.27 (m, 1H), 1.23-1.09 (m, 10H), 1.08-1.00 (m, 1H), 0.86 (s, 3H), 0.65-0.57 (m, 3H). LCMS (M+1)=659.31.

### Example 91

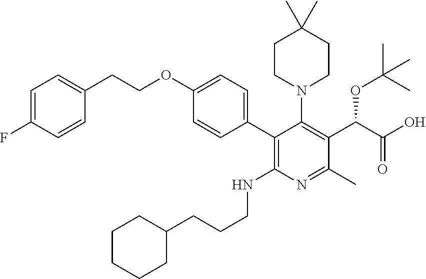 [SMILES: Cc1nc(NCCCC2CCCCC2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(6-((3-cyclohexylpropyl)amino)-4-(4, 4-dimethylpiperidin-1-yl)-5-(4-(4-fluorophenethoxy)phenyl)-2-methylpyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol) and 3-cyclohexylpropan-1-amine (0.015 g, 0.108 mmol) in NMP (1 mL) was heated at 180° C. for 2 h. Ethanol (0.5 mL) and 5 M NaOH (0.054 mL, 0.271 mmol) were added and the mixture was heated at 80° C. for 4.5 h, cooled to ambient temperature, and filtered. The crude mixture was purified via preparative HPLC to afford the desired product (5.3 mg, 28%). LCMS (M+1)=688.41.

### Example 92

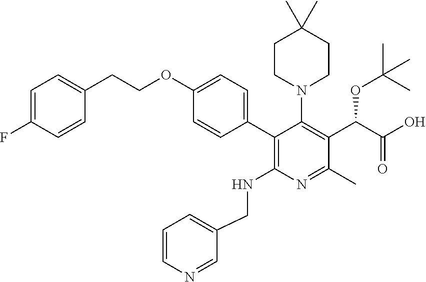 [SMILES: Cc1nc(NCc2cccnc2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-((pyridin-3-ylmethyl)amino)pyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol) and pyridin-3-ylmethanamine (0.012 g, 0.108 mmol) in NMP (1 mL) was heated at 180° C. for 2 h. Ethanol (0.5 mL) and 5 M NaOH (0.054 mL, 0.271 mmol) were added and the mixture was heated at 80° C. for 4.5 h, cooled to ambient temperature, and filtered. The crude mixture was purified via preparative HPLC to afford the desired product (2.2 mg, 12%). ¹H NMR (500 MHz, DMSO-d6) δ 8.52-8.43 (m, 1H), 8.36 (br d, J=3.7 Hz, 1H), 7.69-7.59 (m, 1H), 7.36 (dd, J=8.4, 5.5 Hz, 2H), 7.29-7.24 (m, 1H), 7.21-7.16 (m, 1H), 7.14-7.02 (m, 6H), 5.74 (s, 1H), 5.15 (t, J=6.2 Hz, 1H), 4.44 (d, J=5.9 Hz, 2H), 4.28-4.21 (m, 2H), 3.05 (t, J=6.6 Hz, 2H), 2.55 (s, 2H), 2.32 (s, 3H), 1.53-1.44 (m, 1H), 1.36-1.25 (m, 1H), 1.19-1.09 (m, 11H), 1.04-0.97 (m, 1H), 0.87-0.79 (m, 3H), 0.63-0.54 (m, 3H). LCMS (M+1)=655.33.

### Example 93

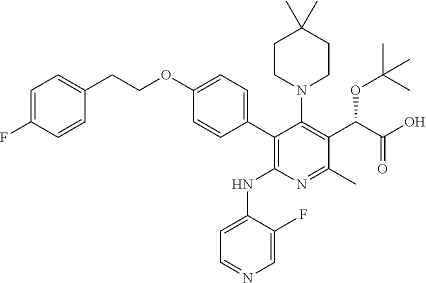 [SMILES: Cc1nc(Nc2ccncc2F)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-6-((3-fluoropyridin-4-yl)amino)-2-methylpyridin-3-yl)acetic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.025 g, 0.041 mmol), 4-bromo-3-fluoropyridine (8.72 mg, 0.050 mmol), Xantphos (1.433 mg, 2.476 mol, tris(dibenzylideneacetone)dipalladium(0) (0.756 mg, 0.825 μmol), and cesium carbonate (0.019 g, 0.058 mmol) in dioxane (1 mL) was degassed and heated at 100° C. for 5 h. The reaction mixture was cooled to ambient temperature, filtered through a pad of celite, and concentrated in vacuo. The residue was taken up in ethanol (1 mL) and 5 M NaOH (0.083 mL, 0.413 mmol) was added. The mixture was heated at 100° C. for 1 h, cooled to ambient temperature, and filtered. The crude mixture was purified via preparative HPLC to afford the desired product (8.2 mg, 30%). LCMS (M+1)=659.29.

### Example 94

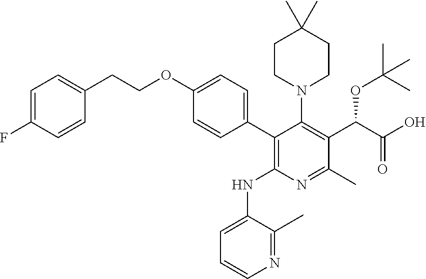 [SMILES: Cc1ncccc1Nc1nc(C)c([C@H](OC(C)(C)C)C(=O)O)c(N2CCC(C)(C)CC2)c1-c1ccc(OCCc2ccc(F)cc2)cc1]

### (S)-2-(tert-Butoxy)-2-(4-(4, 4-dimethylpiperidin-1-yl)-5-(4-(4-fluorophenethoxy)phenyl)-2-methyl-6-((2-methylpyridin-3-yl)amino)pyridin-3-yl)acetic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.025 g, 0.041 mmol), 3-bromo-2-methylpyridine (8.52 mg, 0.050 mmol), Xantphos (1.433 mg, 2.476 μmol), tris(dibenzylideneacetone)dipalladium(0) (0.756 mg, 0.825 μmol), and cesium carbonate (0.019 g, 0.058 mmol) in dioxane (1 mL) was degassed and heated at 100° C. for 5 h. The reaction mixture was cooled to ambient temperature, filtered through a pad of celite, and concentrated in vacuo. The residue was taken up in ethanol (1 mL) and 5 M NaOH (0.083 mL, 0.413 mmol) was added. The mixture was heated at 100° C. for 1 h, cooled to ambient temperature, and filtered. The crude mixture was purified via preparative HPLC to afford the desired product (18.2 mg, 67%). LCMS (M+1)=655.4.

### Example 95

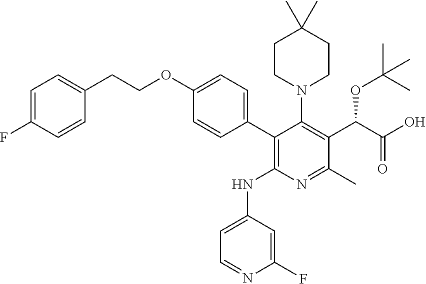 [SMILES: Cc1nc(Nc2ccnc(F)c2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-6-((2-fluoropyridin-4-yl)amino)-2-methylpyridin-3-yl)acetic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.025 g, 0.041 mmol), 4-bromo-2-fluoropyridine (8.72 mg, 0.050 mmol), Xantphos (1.433 mg, 2.476 μmol), tris(dibenzylideneacetone)dipalladium(0) (0.756 mg, 0.825 μmol) and cesium carbonate (0.019 g, 0.058 mmol) in dioxane (1 mL) was degassed and heated at 100° C. for 5 h. The reaction mixture was cooled to ambient temperature, filtered through a pad of celite, and concentrated in vacuo. The residue was taken up in ethanol (1 mL) and 5 M NaOH (0.083 mL, 0.413 mmol) was added. The mixture was heated at 100° C. for 1 h, cooled to ambient temperature, and filtered. The crude mixture was purified via preparative HPLC to afford the desired product (9.5 mg, 35%). LCMS (M+1)=659.29.

### Example 96

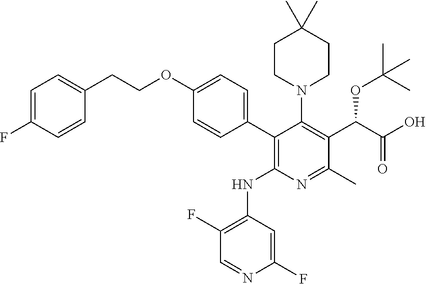 [SMILES: Cc1nc(Nc2cc(F)ncc2F)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-6-((2-fluoropyridin-4-yl)amino)-2-methylpyridin-3-yl)acetic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.025 g, 0.041 mmol), 4-bromo-2-fluoropyridine (8.72 mg, 0.050 mmol), Xantphos (1.433 mg, 2.476 μmol), tris(dibenzylideneacetone)dipalladium(0) (0.756 mg, 0.825 μmol), and cesium carbonate (0.019 g, 0.058 mmol) in dioxane (1 mL) was degassed and heated at 100° C. for 5 h. The reaction mixture was cooled to ambient temperature, filtered through a pad of celite, and concentrated in vacuo. The residue was taken up in ethanol (1 mL) and 5 M NaOH (0.083 mL, 0.413 mmol) was added. The mixture was heated at 100° C. for 1 h, cooled to ambient temperature, and filtered. The crude mixture was purified via preparative HPLC to afford the desired product (9.5 mg, 35%). LCMS (M+1)=659.4.

### Example 97

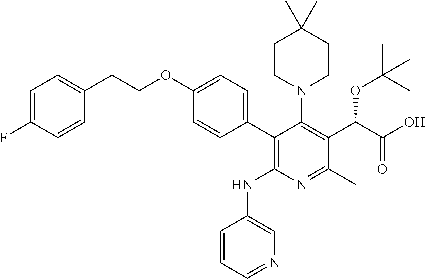 [SMILES: Cc1nc(Nc2cccnc2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-((pyridin-2-ylmethyl)amino)pyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol), and pyridin-2-ylmethanamine (0.012 g, 0.108 mmol) in NMP (1 mL) was heated at 180° C. for 5 h. Ethanol and 5 M NaOH (0.054 mL, 0.271 mmol) were added and the mixture was heated at 80° C. for 4.5 h, cooled to ambient temperature, and filtered. The crude mixture was purified via preparative HPLC to afford the desired product (9.8 mg, 55%). ¹H NMR (500 MHz, DMSO-d₆) δ 8.45-8.28 (m, 1H), 7.77-7.61 (m, 1H), 7.44-6.88 (m, 11H), 5.75 (s, 1H), 5.36-5.20 (m, 1H), 4.59-4.44 (m, 2H), 4.34-4.18 (m, 2H), 3.10-3.00 (m, 2H), 2.84-2.75 (m, 1H), 2.35-2.21 (m, 4H), 1.55-1.45 (m, 1H), 1.37-1.25 (m, 1H), 1.20-1.08 (m, 10H), 1.05-0.98 (m, 1H), 0.88-0.79 (m, 3H), 0.68-0.55 (m, 3H). LCMS (M+1)=655.4.

### Example 98

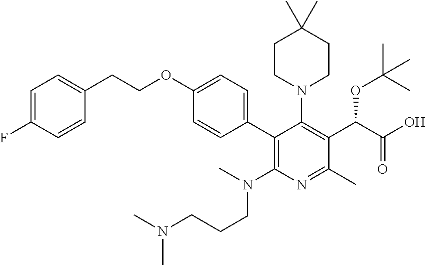 [SMILES: Cc1nc(N(C)CCCN(C)C)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(6-((3-(dimethylamino)propyl)(methyl)amino)-4-(4, 4-dimethylpiperidin-1-yl)-5-(4-(4-fluorophenethoxy)phenyl)-2-methylpyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol) and N1,N1,N3-trimethylpropane-1,3-diamine (3.15 mg, 0.027 mmol) in NMP (1 mL) was heated at 180° C. for 5 h. Ethanol (mL) and 5 M NaOH (0.054 mL, 0.271 mmol) were added and the mixture was heated at 80° C. for 4.5 h, cooled to ambient temperature, and filtered. The crude mixture was purified via preparative HPLC to afford the desired product (4.5 mg, 25%). LCMS (M+1)=663.4.

### Example 99

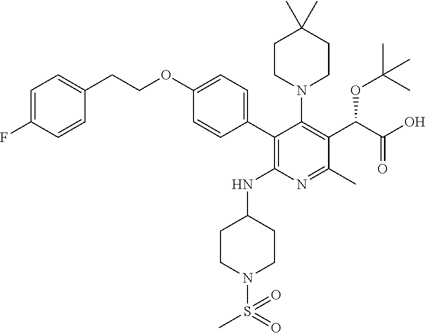 [SMILES: Cc1nc(NC2CCN(S(C)(=O)=O)CC2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-2-methyl-6-((1-(methylsulfonyl)piperidin-4-yl)amino)pyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol), 1-(methylsulfonyl)piperidin-4-amine (0.019 g, 0.108 mmol), and sodium tert-butoxide (5.20 mg, 0.054 mmol) in NMP (1 mL) was heated at 180° C. for 5 h and cooled to ambient temperature. Ethanol (0.5 mL) and 5 M NaOH (0.054 mL, 0.271 mmol) were added to the reaction mixture and heated at 800 C for 2 h, cooled to ambient temperature, and filtered. The crude mixture was purified via preparative HPLC to afford the desired product (2.9 mg, 15%). LCMS (M+1)=725.29.

### Example 100

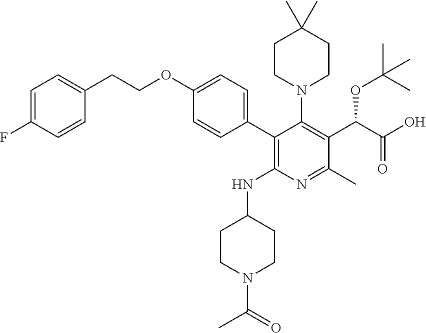 [SMILES: CC(=O)N1CCC(Nc2nc(C)c([C@H](OC(C)(C)C)C(=O)O)c(N3CCC(C)(C)CC3)c2-c2ccc(OCCc3ccc(F)cc3)cc2)CC1]

### (S)-2-(6-((1-Acetylpiperidin-4-yl)amino)-4-(4, 4-dimethylpiperidin-1-yl)-5-(4-(4-fluorophenethoxy)phenyl)-2-methylpyridin-3-yl)-2-(tert-butoxy)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.02 g, 0.027 mmol), 1-(4-aminopiperidin-1-yl)ethanone (0.015 g, 0.108 mmol and sodium tert-butoxide (5.20 mg, 0.054 mmol) in NMP (1 mL) was heated at 180° C. for 5 h. The reaction was then cooled to ambient temperature. Ethanol (0.5 mL) and 5 M NaOH (0.054 mL, 0.271 mmol) were added to the reaction mixture and heated at 800 C for 2 h, cooled to ambient temperature, and filtered. The crude mixture was purified via preparative HPLC to afford the desired product (2.7 mg, 14%). LCMS (M+1)=689.3.

### Example 101

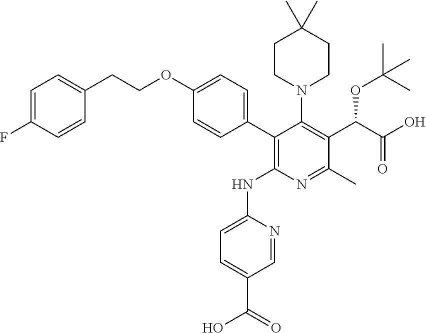 [SMILES: Cc1nc(Nc2ccc(C(=O)O)cn2)c(-c2ccc(OCCc3ccc(F)cc3)cc2)c(N2CCC(C)(C)CC2)c1[C@H](OC(C)(C)C)C(=O)O]

### (S)-6-((5-(tert-Butoxy(carboxy)methyl)-4-(4, 4-dimethylpiperidin-1-yl)-3-(4-(4-fluorophenethoxy)phenyl)-6-methylpyridin-2-yl)amino)nicotinic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.025 g, 0.041 mmol), 2-bromo-5-cyanopyridine (9.06 mg, 0.050 mmol), Xantphos (1.433 mg, 2.476 μmol), tris(dibenzylideneacetone)dipalladium(0) (0.756 mg, 0.825 μmol), and cesium carbonate (0.019 g, 0.058 mmol) in dioxane (1 mL) was degassed and heated at 100° C. for 3 h. The reaction mixture was cooled to ambient temperature, filtered through a pad of celite, and concentrated in vacuo. The residue was taken up in ethanol (1 mL) and 5 M NaOH (0.083 ml, 0.413 mmol) was added. The mixture was heated at 80° C. for 2.5 h, cooled to ambient temperature, and filtered. The crude mixture was purified via preparative HPLC to afford the desired product (9.2 mg, 32%). LCMS (M+1)=685.2.

### Example 102

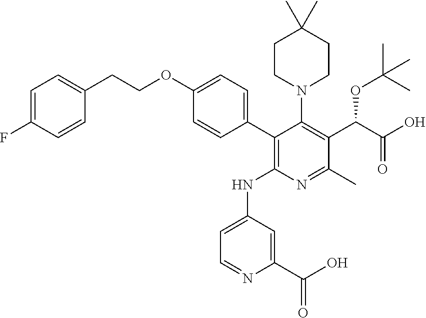 [SMILES: Cc1nc(Nc2ccnc(C(=O)O)c2)c(-c2ccc(OCCc3ccc(F)cc3)cc2)c(N2CCC(C)(C)CC2)c1[C@H](OC(C)(C)C)C(=O)O]

### (S)-4-((5-(tert-Butoxy(carboxy)methyl)-4-(4, 4-dimethylpiperidin-1-yl)-3-(4-(4-fluorophenethoxy)phenyl)-6-methylpyridin-2-yl)amino)picolinic acid

A mixture of (S)-isopropyl 2-(6-amino-4-(4,4-dimethylpiperidin-1-yl)-5-(4-(4-fluorophenethoxy)phenyl)-2-methylpyridin-3-yl)-2-(tert-butoxy)acetate (0.025 g, 0.041 mmol), 4-bromo-2-cyanopyridine (9.06 mg, 0.050 mmol), Xantphos (1.433 mg, 2.476 μmol), tris(dibenzylideneacetone)dipalladium(0) (0.756 mg, 0.825 μmol), and cesium carbonate (0.019 g, 0.058 mmol) in dioxane (1 mL) was degassed and heated at 100° C. for 3 h. The reaction mixture was cooled to ambient temperature, filtered through a pad of celite, and concentrated in vacuo. The residue was taken up in ethanol (1 mL) and 5 M NaOH (0.083 ml, 0.413 mmol) was added. The mixture was heated at 80° C. for 2.5 h, cooled to ambient temperature, and filtered. The crude mixture was purified via preparative HPLC to afford the desired product (20.1 mg, 70%). LCMS (M+1)=685.2.

### Example 103

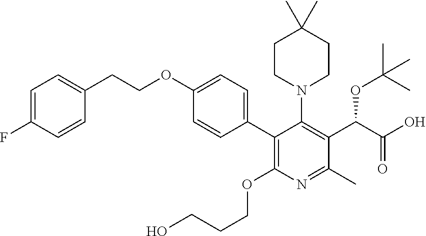 [SMILES: Cc1nc(OCCCO)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)phenyl)-6-(2-hydroxyethoxy)-2-methylpyridin-3-yl)acetic acid

NaH (0.012 g, 0.297 mmol) was added to a solution of (S)-isopropyl 2-(tert-butoxy)-2-(4-(4,4-dimethylpiperidin-1-yl)-5-(4-(4-fluorophenethoxy)phenyl)-6-hydroxy-2-methylpyridin-3-yl)acetate (0.03 g, 0.049 mmol) in DMF (1 mL). The mixture was stirred for 15 min and 2-bromoethanol (0.019 g, 0.148 mmol) was added. Stirring was continued at ambient temperature for 18 h. The reaction mixture was heated at 80° C. for 1.5 h and cooled to ambient temperature. Ethanol (1 mL) and 5 M NaOH (0.099 mL, 0.494 mmol) were added and the mixture was heated at 80° C. for 2 h. After cooling to ambient temperature, the reaction was filtered. The crude mixture was purified via preparative HPLC to afford the desired product (5.8 mg, 19%). LCMS (M+1)=609.4.

### Example 104

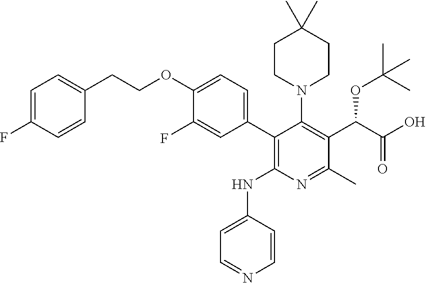 [SMILES: Cc1nc(Nc2ccncc2)c(-c2ccc(OCCc3ccc(F)cc3)c(F)c2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(3-fluoro-4-(4-fluorophenethoxy)phenyl)-2-methyl-6-(pyridin-4-ylamino)pyridin-3-yl)acetic acid

A mixture of (S)-isopropyl 2-(6-amino-4-(4,4-dimethylpiperidin-1-yl)-5-(3-fluoro-4-(4-fluorophenethoxy)phenyl)-2-methylpyridin-3-yl)-2-(tert-butoxy)acetate (0.025 g, 0.040 mmol), 4-bromopyridine hydrochloride (9.35 mg, 0.048 mmol), Xantphos (1.391 mg, 2.405 μmol), tris(dibenzylideneacetone)dipalladium(0) (0.734 mg, 0.802 mol), and sodium tert-butoxide (9.24 mg, 0.096 mmol) in dioxane (1 mL) was degassed and heated at 100° C. for 5 h. The reaction mixture was cooled to ambient temperature, filtered through a pad of celite, and concentrated in vacuo. The residue was taken up in ethanol (1 mL) and 5 M NaOH (0.080 ml, 0.401 mmol) was added. The mixture was heated at 80° C. for 3 h, cooled to ambient temperature, and filtered. The crude mixture was purified via preparative HPLC to afford the desired product (9.4 mg, 36%). LCMS (M+1)=659.4.

### Example 105

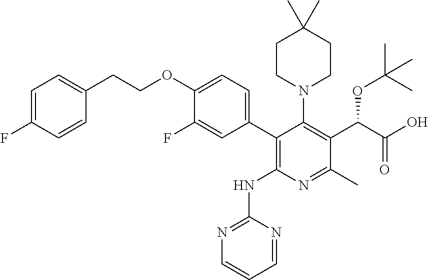 [SMILES: Cc1nc(Nc2ncccn2)c(-c2ccc(OCCc3ccc(F)cc3)c(F)c2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(3-fluoro-4-(4-fluorophenethoxy)phenyl)-2-methyl-6-(pyrimidin-2-ylamino)pyridin-3-yl)acetic acid

A mixture of (S)-isopropyl 2-(6-amino-4-(4,4-dimethylpiperidin-1-yl)-5-(3-fluoro-4-(4-fluorophenethoxy)phenyl)-2-methylpyridin-3-yl)-2-(tert-butoxy)acetate (0.025 g, 0.040 mmol), 2-bromopyrimidine (7.65 mg, 0.048 mmol), Xantphos (1.391 mg, 2.405 μmol), tris(dibenzylideneacetone)dipalladium(0) (0.734 mg, 0.802 μmol), and cesium carbonate (0.018 g, 0.056 mmol) in dioxane (1 mL) was degassed and heated at 100° C. for 5 h. The reaction mixture was cooled to ambient temperature, filtered through a pad of celite, and concentrated in vacuo. The residue was taken up in ethanol (1 mL) and 5 M NaOH (0.080 mL, 0.401 mmol) was added. The mixture was heated at 80° C. for 3 h, cooled to ambient temperature, and filtered. The crude mixture was purified via preparative HPLC to afford the desired product (21.4 mg, 81%). LCMS (M+1)=660.3.

### Example 106

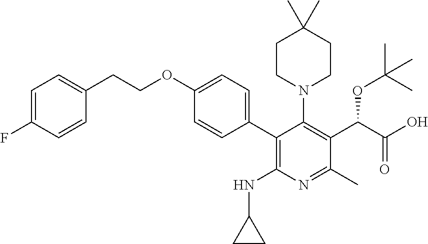 [SMILES: Cc1nc(NC2CC2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(6-(cyclopropylamino)-4-(4, 4-dimethylpiperidin-1-yl)-5-(4-(4-fluorophenethoxy)phenyl)-2-methylpyridin-3-yl)acetic acid

A mixture of (S)-isopropyl 2-(tert-butoxy)-2-(4-(4,4-dimethylpiperidin-1-yl)-5-(4-(4-fluorophenethoxy)phenyl)-2-methyl-6-(((trifluoromethyl)sulfonyl)oxy)pyridin-3-yl)acetate (0.025 g, 0.034 mmol) and cyclopropanamine (7.73 mg, 0.135 mmol) in NMP (1 mL) was heated at 180° C. for 3 h (LCMS showed the desired product peak as the major peak). Ethanol and 5 M NaOH (0.068 mL, 0.338 mmol) were added. The mixture was heated at 80° C. for 3 h, cooled to ambient temperature, and filtered. The crude mixture was purified via preparative HPLC to afford the desired product (2.6 mg, 12%). LCMS (M+1)=604.4.

### Example 107

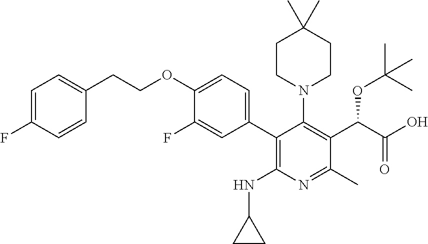 [SMILES: Cc1nc(NC2CC2)c(-c2ccc(OCCc3ccc(F)cc3)c(F)c2)c(N2CCC(C)(C)CC2)c1[C@H](OC(C)(C)C)C(=O)O]

### (S)-2-(tert-Butoxy)-2-(6-(cyclopropylamino)-4-(4, 4-dimethylpiperidin-1-yl)-5-(3-fluoro-4-(4-fluorophenethoxy)phenyl)-2-methylpyridin-3-yl)acetic acid

A mixture of (S)-isopropyl 2-(tert-butoxy)-2-(4-(4,4-dimethylpiperidin-1-yl)-5-(3-fluoro-4-(4-fluorophenethoxy)phenyl)-2-methyl-6-(((trifluoromethyl)sulfonyl)oxy)pyridin-3-yl)acetate (0.025 g, 0.033 mmol) and cyclopropanamine (0.038 g, 0.661 mmol) in NMP (1 mL) was heated at 180° C. for 10 h (LCMS showed the desired product peak as the major peak). Ethanol and 5 M NaOH (0.066 mL, 0.330 mmol) were added and the mixture was heated at 80° C. for 3 h, cooled to ambient temperature, and filtered. The crude mixture was purified via preparative HPLC to afford the desired product (1.1 mg, 5%). LCMS (M+1)=622.4.

### Example 108

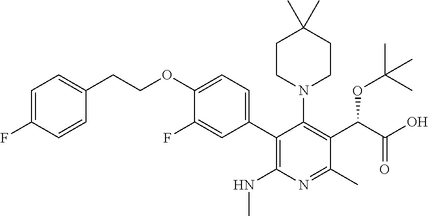 [SMILES: CNc1nc(C)c([C@H](OC(C)(C)C)C(=O)O)c(N2CCC(C)(C)CC2)c1-c1ccc(OCCc2ccc(F)cc2)c(F)c1]

### (S)-2-(tert-Butoxy)-2-(4-(4, 4-dimethylpiperidin-1-yl)-5-(3-fluoro-4-(4-fluorophenethoxy)phenyl)-2-methyl-6-(methylamino)pyridin-3-yl)acetic acid

A mixture of (S)-isopropyl 2-(tert-butoxy)-2-(4-(4,4-dimethylpiperidin-1-yl)-5-(3-fluoro-4-(4-fluorophenethoxy)phenyl)-2-methyl-6-(((trifluoromethyl)sulfonyl)oxy)pyridin-3-yl)acetate (0.025 g, 0.033 mmol) and methanamine (0.311 g, 3.30 mmol) in NMP (1 mL) was heated at 180° C. for 22 h (LCMS showed the desired product peak as the major peak). Ethanol and 5 M NaOH (0.066 mL, 0.330 mmol) was added and the mixture was heated at 80° C. for 2 h, cooled to ambient temperature, and filtered. The crude mixture was purified via preparative HPLC to afford the desired product (3.6 mg, 18%). LCMS (M+1)=622.4.

### Example 109

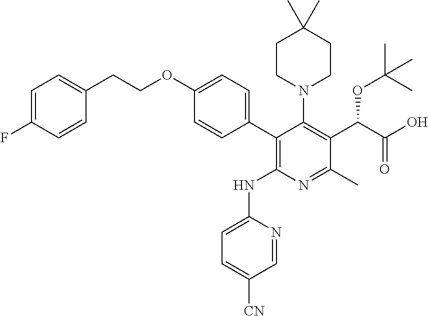 [SMILES: Cc1nc(Nc2ccc(C#N)cn2)c(-c2ccc(OCCc3ccc(F)cc3)cc2)c(N2CCC(C)(C)CC2)c1[C@H](OC(C)(C)C)C(=O)O]

### (S)-2-(tert-Butoxy)-2-(6-((5-cyanopyridin-2-yl)amino)-4-(4, 4-dimethylpiperidin-1-yl)-5-(4-(4-fluorophenethoxy)phenyl)-2-methylpyridin-3-yl)acetic acid

A mixture of (S)-2-(6-amino-4-(4,4-dimethylpiperidin-1-yl)-5-(4-(4-fluorophenethoxy)phenyl)-2-methylpyridin-3-yl)-2-(tert-butoxy)acetic acid (0.03 g, 0.053 mmol), 2-bromo-5-cyanopyridine (0.012 g, 0.064 mmol), Xantphos (1.848 mg, 3.19 μmol), tris(dibenzylideneacetone)dipalladium(0) (0.975 mg, 1.064 μmol), and cesium carbonate (0.024 g, 0.075 mmol) in dioxane (1 mL) was degassed and heated at 100° C. for 3 h. The reaction mixture was cooled to ambient temperature, filtered through a pad of celite, and concentrated in vacuo. The residue was taken up in methanol and the crude mixture was purified via preparative HPLC to afford the desired product (20.2 mg, 57%. LCMS (M+1)=666.3.

### Example 110

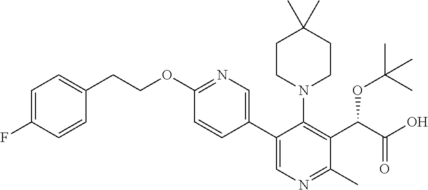 [SMILES: Cc1ncc(-c2ccc(OCCc3ccc(F)cc3)nc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-6′-((4-fluorophenethyl)amino)-6-methyl-[3, 3′-bipyridin]-5-yl)acetic acid

A solution of (S)-isopropyl 2-(tert-butoxy)-2-(4-(4,4-dimethylpiperidin-1-yl)-6′-fluoro-6-methyl-[3,3′-bipyridin]-5-yl)acetate (33 mg, 0.070 mmol, 1 equiv) and 2-(4-fluorophenyl)ethanamine (29 mg, 0.21 mmol, 3 equiv) in acetonitrile (2.3 mL) was heated at 120° C. for 18 h. Upon cooling to ambient temperature, the reaction was concentrated. The residue was taken up in 9:1 EtOH: 10 N NaOH and heated at 80° C. for 1 h. After cooling to ambient temperature, the reaction was filtered. The crude mixture was purified by preparative LC/MS to provide the product (13.7 mg, 34%). ¹H NMR (500 MHz, DMSO-d6) δ 8.03-7.96 (m, 1H), 7.90 (s, 1H), 7.35-7.24 (m, 3H), 7.12-7.05 (m, 2H), 6.61-6.48 (m, 2H), 5.82 (s, 1H), 3.58-3.32 (m, 2H), 2.94-2.81 (m, 2H), 2.49 (s, 3H), 1.60-1.16 (m, 4H), 1.13 (s, 9H), 0.84 (br s, 6H) [note: piperidine protons are broad and not all visible]. LCMS (M+1)=549.3.

### Example 111

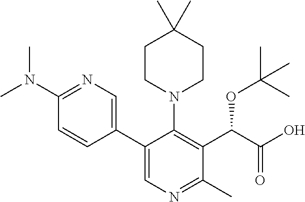 [SMILES: Cc1ncc(-c2ccc(N(C)C)nc2)c(N2CCC(C)(C)CC2)c1[C@H](OC(C)(C)C)C(=O)O]

### (S)-2-(tert-Butoxy)-2-(6′-(dimethylamino)-4-(4, 4-dimethylpiperidin-1-yl)-6-methyl-[3, 3′-bipyridin]-5-yl)acetic acid

A solution of (S)-isopropyl 2-(tert-butoxy)-2-(4-(4,4-dimethylpiperidin-1-yl)-6′-fluoro-6-methyl-[3,3′-bipyridin]-5-yl)acetate (31 mg, 0.066 mmol, 1 equiv) in DMF (2 mL), EtOH (2 mL), and 10 N NaOH (0.2 mL) was heated at 90° C. for 2 h. After cooling to ambient temperature, the reaction was filtered. The crude mixture was purified by preparative LC/MS to provide the product (7.6 mg, 25%) and (S)-2-(tert-butoxy)-2-(4-(4,4-dimethylpiperidin-1-yl)-6′-ethoxy-6-methyl-[3,3′-bipyridin]-5-yl)acetic acid (8.7 mg, 28%). ¹H NMR (500 MHz, DMSO-d₆) δ 8.10-7.90 (m, 2H), 7.44 (dd, J=8.6, 2.4 Hz, 1H), 6.72 (d, J=8.8 Hz, 1H), 5.87 (s, 1H), 3.07 (s, 6H), 2.50 (br s, 3H), 1.33 (br s, 4H), 1.14 (s, 9H), 0.84 (br s, 6H) [note: piperidine protons are broad and not all visible]. LCMS (M+1)=455.2.

### Example 112

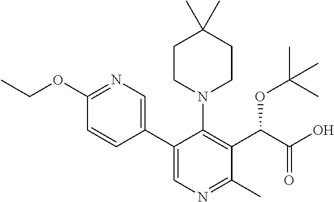 [SMILES: CCOc1ccc(-c2cnc(C)c([C@H](OC(C)(C)C)C(=O)O)c2N2CCC(C)(C)CC2)cn1]

### (S)-2-(tert-Butoxy)-2-(4-(4, 4-dimethylpiperidin-1-yl)-6′-ethoxy-6-methyl-[3, 3′-bipyridin]-5-yl)acetic acid

Isolated in the same reaction as (S)-2-(tert-butoxy)-2-(6′-(dimethylamino)-4-(4,4-dimethylpiperidin-1-yl)-6-methyl-[3,3′-bipyridin]-5-yl)acetic acid in 28% yield. ¹H NMR (500 MHz, DMSO-d₆) δ 8.09 (d, J=1.5 Hz, 1H), 8.06 (s, 1H), 7.65 (dd, J=8.4, 2.2 Hz, 1H), 6.89 (d, J=8.4 Hz, 1H), 5.85 (s, 1H), 4.38 (t, J=7.0 Hz, 2H), 2.51 (s, 3H), 1.40-1.22 (m, 4H), 1.14 (s, 9H), 0.82 (br s, 6H) [note: piperidine protons are broad and not all visible]. LCMS (M+1)=456.2.

### Example 113

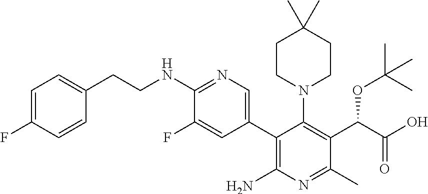 [SMILES: Cc1nc(N)c(-c2cnc(NCCc3ccc(F)cc3)c(F)c2)c(N2CCC(C)(C)CC2)c1[C@H](OC(C)(C)C)C(=O)O]

### (S)-2-(2-Amino-4-(4, 4-dimethylpiperidin-1-yl)-5′-fluoro-6′-((4-fluorophenethyl)amino)-6-methyl-[3, 3′-bipyridin]-5-yl)-2-(tert-butoxy)acetic acid

A solution of (S)-isopropyl 2-(2-amino-4-(4,4-dimethylpiperidin-1-yl)-5′,6′-difluoro-6-methyl-[3,3′-bipyridin]-5-yl)-2-(tert-butoxy)acetate (0.031 g, 0.061 mmol, 1 equiv) and 2-(4-fluorophenyl)ethanamine (0.043 g, 0.307 mmol, 5 equiv) in DMF (1.5 mL) was heated at 100° C. for 3 h. Upon cooling to ambient temperature, the reaction was partitioned between EtOAc and brine. The EtOAc layer was dried (Na₂SO₄) and concentrated in vacuo. The crude intermediate was taken up in EtOH (0.9 mL) and 10 N NaOH (0.1 mL) and heated at 80° C. for 4 h. After cooling to ambient temperature, the reaction was filtered. The crude mixture was purified by preparative LC/MS to provide the product (5.5 mg, 15%) and (S)-2-(tert-butoxy)-2-(6′-(dimethylamino)-4-(4,4-dimethylpiperidin-1-yl)-5′-fluoro-6-methyl-[3,3′-bipyridin]-5-yl)acetic acid (12.1 mg, 39%). ¹H NMR (500 MHz, DMSO-d₆) δ 7.75-7.60 (m, 1H), 7.26 (br d, J=7.3 Hz, 2H), 7.13-7.06 (m, 2H), 6.61 (br s, 1H), 5.72 (s, 1H), 4.99 (br s, 1H), 3.75-3.49 (m, 2H), 2.90 (brt, J=7.0 Hz, 2H), 2.84-2.76 (m, 1H), 2.32 (s, 3H), 2.29-2.23 (m, 1H), 1.57-1.45 (m, 1H), 1.39-1.28 (m, 1H), 1.21-1.18 (m, 1H), 1.16 (s, 9H), 1.13-1.07 (m, 1H), 0.87 (br s, 3H), 0.66 (br s, 3H) [note: piperidine protons are broad and not all visible]. LCMS (M+1)=582.3.

### Example 114

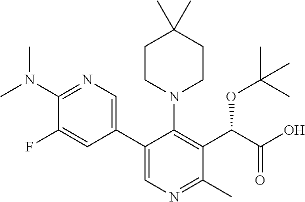 [SMILES: Cc1ncc(-c2cnc(N(C)C)c(F)c2)c(N2CCC(C)(C)CC2)c1[C@H](OC(C)(C)C)C(=O)O]

### (S)-2-(tert-Butoxy)-2-(6′-(dimethylamino)-4-(4, 4-dimethylpiperidin-1-yl)-5′-fluoro-6-methyl-[3,3′-bipyridin]-5-yl)acetic acid

Isolated as a byproduct from same reaction as (S)-2-(2-amino-4-(4,4-dimethylpiperidin-1-yl)-5′-fluoro-6′-((4-fluorophenethyl)amino)-6-methyl-[3,3′-bipyridin]-5-yl)-2-(tert-butoxy)acetic acid in 39% yield. ¹H NMR (500 MHz, DMSO-d6) δ 7.73 (br s, 1H), 7.42-7.17 (m, 1H), 5.72 (s, 1H), 5.01 (s, 1H), 3.07 (d, J=1.5 Hz, 3H), 2.85-2.71 (m, 1H), 2.32 (s, 3H), 2.31-2.25 (m, 1H), 1.58-1.42 (m, 1H), 1.40-1.28 (m, 1H), 1.21-1.18 (m, 1H), 1.16 (s, 9H), 1.13-1.11 (m, 1H), 0.87 (br d, J=0.7 Hz, 3H), 0.65 (br d, J=1.5 Hz, 3H) [note: piperidine protons are broad and not all visible; some integrations may be off from water solvent suppression]. LCMS (M+1)=488.3.

### Example 115

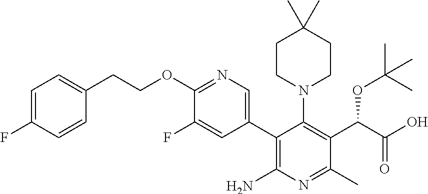 [SMILES: Cc1nc(N)c(-c2cnc(OCCc3ccc(F)cc3)c(F)c2)c(N2CCC(C)(C)CC2)c1[C@H](OC(C)(C)C)C(=O)O]

### (S)-2-(2-Amino-4-(4, 4-dimethylpiperidin-1-yl)-5′-fluoro-6′-(4-fluorophenethoxy)-6-methyl-[3, 3′-bipyridin]-5-yl)-2-(tert-butoxy)acetic acid

To a solution of (S)-isopropyl 2-(2-amino-4-(4,4-dimethylpiperidin-1-yl)-5′,6′-difluoro-6-methyl-[3,3′-bipyridin]-5-yl)-2-(tert-butoxy)acetate (0.055 g, 0.109 mmol, 1 equiv) and 2-(4-fluorophenyl)ethanol (0.076 g, 0.545 mmol, 5 equiv) in THF (2.7 mL) was added 60% NaH (0.022 g, 0.545 mmol, 5 equiv). The reaction was heated at 60° C. for 1 h. EtOH (0.9 mL) and 10 N NaOH (0.1 mL) were added and the temperature was raised to 80° C. for 4 h. After cooling to ambient temperature, the reaction was filtered. The crude mixture was purified by preparative LC/MS to provide the product (15 mg, 24%). ¹H NMR (500 MHz, DMSO-d₆) δ 7.74 (br s, 1H), 7.67-7.44 (m, 1H), 7.38-7.30 (m, 2H), 7.12 (br t, J=8.4 Hz, 2H), 5.70 (br s, 1H), 5.09 (br s, 1H), 4.70-4.56 (m, 2H), 3.12-3.04 (m, 2H), 2.84-2.62 (m, 1H), 2.33 (s, 3H), 1.55-1.44 (m, 1H), 1.40-1.30 (m, 1H), 1.22-1.17 (m, 1H), 1.16 (s, 9H), 1.10-1.02 (m, 1H), 0.87 (br s, 3H), 0.62 (br s, 3H) [note: piperidine protons are broad and not all visible]. LCMS (M+1)=583.3.

### Example 116

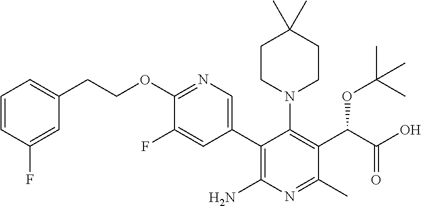 [SMILES: Cc1nc(N)c(-c2cnc(OCCc3cccc(F)c3)c(F)c2)c(N2CCC(C)(C)CC2)c1[C@H](OC(C)(C)C)C(=O)O]

### (S)-2-(2-Amino-4-(4, 4-dimethylpiperidin-1-yl)-5′-fluoro-6′-(3-fluorophenethoxy)-6-methyl-[3, 3′-bipyridin]-5-yl)-2-(tert-butoxy)acetic acid

To a solution of (S)-isopropyl 2-(2-amino-4-(4,4-dimethylpiperidin-1-yl)-5′,6′-difluoro-6-methyl-[3,3′-bipyridin]-5-yl)-2-(tert-butoxy)acetate (0.042 g, 0.059 mmol, 1 equiv) and 2-(3-fluorophenyl)ethanol (0.042 g, 0.297 mmol, 5 equiv) in THF (1.5 mL) was added 60% NaH (0.012 g, 0.297 mmol, 5 equiv). The reaction was heated at 60° C. for 1 h. EtOH (0.9 mL) and 10 N NaOH (0.1 mL) were added and the temperature was raised to 80° C. for 4 h. After cooling to ambient temperature, the reaction was filtered. The crude mixture was purified by preparative LC/MS to provide the product (9 mg, 26%). ¹H NMR (500 MHz, DMSO-d₆) δ 7.89-7.44 (m, 2H), 7.38-7.32 (m, 1H), 7.17-7.10 (m, 2H), 7.07-7.00 (m, 1H), 5.70 (s, 1H), 5.09 (br s, 1H), 4.75-4.53 (m, 2H), 3.18-3.02 (m, 2H), 2.91-2.64 (m, 1H), 2.32 (s, 3H), 1.60-1.43 (m, 1H), 1.26 (s, 2H), 1.16 (s, 9H), 1.09-0.98 (m, 1H), 0.86 (br s, 3H), 0.62 (br s, 3H) [note: appears to be a 60:40 mixture of rotamers around pyridine pyridine biaryl bond, piperidine protons are broad and not all visible]. LCMS (M+1)=583.3.

### Example 117

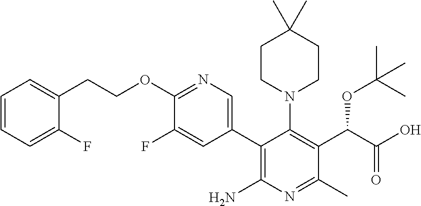 [SMILES: Cc1nc(N)c(-c2cnc(OCCc3ccccc3F)c(F)c2)c(N2CCC(C)(C)CC2)c1[C@H](OC(C)(C)C)C(=O)O]

### (S)-2-(2-Amino-4-(4, 4-dimethylpiperidin-1-yl)-5′-fluoro-6′-(2-fluorophenethoxy)-6-methyl-[3, 3′-bipyridin]-5-yl)-2-(tert-butoxy)acetic acid

To a solution of (S)-isopropyl 2-(2-amino-4-(4,4-dimethylpiperidin-1-yl)-5′,6′-difluoro-6-methyl-[3,3′-bipyridin]-5-yl)-2-(tert-butoxy)acetate (0.057 g, 0.081 mmol, 1 equiv) and 2-(2-fluorophenyl)ethanol (0.057 g, 0.406 mmol, 5 equiv) in THF (1.5 mL) was added 60% NaH (0.016 g, 0.406 mmol, 5 equiv). The reaction was heated at 60° C. for 1 h. EtOH (0.9 mL) and 10 N NaOH (0.1 mL) were added and the temperature was raised to 80° C. for 4 h. After cooling to ambient temperature, the reaction was filtered. The crude mixture was purified by preparative LC/MS to provide the product (25 mg, 52%). ¹H NMR (500 MHz, DMSO-d₆) δ 7.89-7.46 (m, 2H), 7.39 (brt, J=7.5 Hz, 1H), 7.30 (q, J=7.2 Hz, 1H), 7.16 (brt, J=8.4 Hz, 2H), 5.70 (s, 1H), 4.75-4.56 (m, 2H), 3.18-3.10 (m, 1H), 2.82-2.70 (m, 1H), 2.33 (s, 3H), 1.56-1.42 (m, 1H), 1.26 (br s, 2H), 1.16 (s, 9H), 1.10-1.01 (m, 1H), 0.86 (br s, 3H), 0.63 (br s, 3H) [note: appears to be a 60:40 mixture of rotamers around pyridine pyridine biaryl bond, piperidine protons are broad and not all visible]. LCMS (M+1)=583.2.

### Example 118

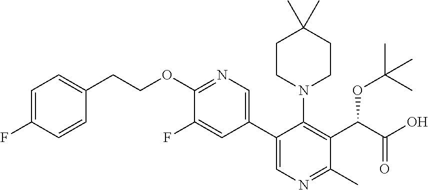 [SMILES: Cc1ncc(-c2cnc(OCCc3ccc(F)cc3)c(F)c2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5′-fluoro-6′-(4-fluorophenethoxy)-6-methyl-[3, 3′-bipyridin]-5-yl)acetic acid

A mixture of 2-(4-fluorophenyl)ethanol (0.052 mL, 0.419 mmol), NaH (0.017 g, 0.419 mmol), and (S)-isopropyl 2-(tert-butoxy)-2-(4-(4,4-dimethylpiperidin-1-yl)-5′,6′-difluoro-6-methyl-[3,3′-bipyridin]-5-yl)acetate (0.041 g, 0.084 mmol) in THF was stirred at ambient temperature for 1 h. Upon completion, 5 M NaOH (0.167 mL, 0.837 mmol) was added and the mixture was heated at 80° C. for 2 h. After cooling to ambient temperature, the reaction was filtered. The crude mixture was purified by preparative LC/MS to provide the product (23.5 mg, 49%). ¹H NMR (500 MHz, DMSO-d₆) δ 8.13-8.04 (m, 1H), 7.93 (d, J=2.2 Hz, 1H), 7.70 (dd, J=11.0, 1.8 Hz, 1H), 7.41-7.27 (m, 2H), 7.17-7.05 (m, 2H), 5.82 (s, 1H), 4.72-4.54 (m, 2H), 3.10 (t, J=6.6 Hz, 1H), 1.36-1.28 (m, 3H), 1.14 (s, 10H), 0.90-0.76 (m, 6H) [note: piperidine protons not all visible]. LCMS (M+1)=568.3.

### Example 119

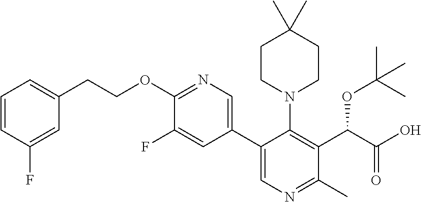 [SMILES: Cc1ncc(-c2cnc(OCCc3cccc(F)c3)c(F)c2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5′-fluoro-6′-(3-fluorophenethoxy)-6-methyl-[3, 3′-bipyridin]-5-yl)acetic acid

A mixture of 2-(3-fluorophenyl)ethanol (0.052 mL, 0.419 mmol), NaH (0.017 g, 0.419 mmol), and (S)-isopropyl 2-(tert-butoxy)-2-(4-(4,4-dimethylpiperidin-1-yl)-5′,6′-difluoro-6-methyl-[3,3′-bipyridin]-5-yl)acetate (0.041 g, 0.084 mmol) in THF was stirred at ambient temperature for 1 h. Then, 5 M NaOH (0.167 mL, 0.837 mmol) was added and the mixture was heated at 80° C. for 2 h. After cooling to ambient temperature, the reaction was filtered. The crude mixture was purified by preparative LC/MS to provide the product (23.4 mg, 49%). ¹H NMR (500 MHz, DMSO-d₆) δ 8.14-8.03 (m, 1H), 7.93 (d, J=1.8 Hz, 1H), 7.70 (dd, J=11.0, 1.8 Hz, 1H), 7.42-7.24 (m, 1H), 7.21-6.97 (m, 3H), 5.83 (s, 1H), 4.67 (t, J=6.6 Hz, 2H), 3.18-3.00 (m, 1H), 1.32 (br s, 3H), 1.14 (s, 10H), 0.89-0.76 (m, 6H) [note: piperidine protons not all visible]. LCMS (M+1)=568.3.

### Example 120

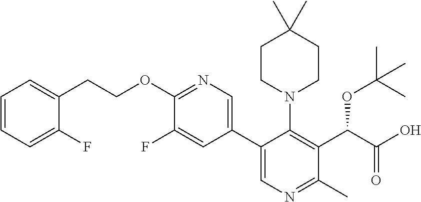 [SMILES: Cc1ncc(-c2cnc(OCCc3ccccc3F)c(F)c2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5′-fluoro-6′-(2-fluorophenethoxy)-6-methyl-[3, 3′-bipyridin]-5-yl)acetic acid

A mixture of 2-(2-fluorophenyl)ethanol (0.056 mL, 0.419 mmol), NaH (0.017 g, 0.419 mmol), and (S)-isopropyl 2-(tert-butoxy)-2-(4-(4,4-dimethylpiperidin-1-yl)-5′,6′-difluoro-6-methyl-[3,3′-bipyridin]-5-yl)acetate (0.041 g, 0.084 mmol) in THF was stirred at ambient temperature for 1 h. Then, 5 M NaOH (0.167 mL, 0.837 mmol) was added and the mixture was heated at 80° C. for 2 h. After cooling to ambient temperature, the reaction was filtered. The crude mixture was purified by preparative LC/MS to provide the product (25 mg, 52%). ¹H NMR (500 MHz, DMSO-d₆) δ 8.09 (s, 1H), 7.92 (d, J=2.2 Hz, 1H), 7.70 (dd, J=11.2, 2.0 Hz, 1H), 7.43-7.35 (m, 1H), 7.34-7.26 (m, 1H), 7.33-7.24 (m, 1H), 7.20-7.07 (m, 2H), 5.83 (s, 1H), 4.74-4.57 (m, 2H), 3.19-3.10 (m, 2H), 2.52 (s, 3H), 1.37-1.25 (m, 4H), 1.15 (s, 9H), 0.83 (br s, 6H) [note: piperidine protons not all visible]. LCMS (M+1)=568.3.

### Example 121

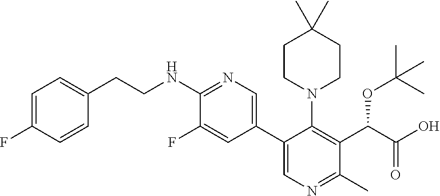 [SMILES: Cc1ncc(-c2cnc(NCCc3ccc(F)cc3)c(F)c2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5′-fluoro-6′-((4-fluorophenethyl)amino)-6-methyl-[3, 3′-bipyridin]-5-yl)acetic acid

A mixture of 2-(4-fluorophenyl)ethanamine (0.055 mL, 0.419 mmol) and (S)-isopropyl 2-(tert-butoxy)-2-(4-(4,4-dimethylpiperidin-1-yl)-5′,6′-difluoro-6-methyl-[3,3′-bipyridin]-5-yl)acetate (0.041 g, 0.084 mmol) in NMP was heated at 100° C. for 2 h and cooled to rt. Then, 5M NaOH (0.167 mL, 0.837 mmol) was added and the mixture was heated at 80° C. for 2 h cooled to rt, filtered and purified by preparative LC/MS to give the product (25.0 mg, 50%). ¹H NMR (500 MHz, DMSO-d₆) δ 8.11-8.01 (m, 1H), 7.84-7.76 (m, 1H), 7.38-7.19 (m, 3H), 7.16-7.02 (m, 2H), 6.67 (brt, J=6.2 Hz, 1H), 5.85 (s, 1H), 3.72-3.54 (m, 2H), 2.91 (t, J=7.2 Hz, 2H), 1.34 (br s, 2H), 1.18-1.07 (m, 10H), 0.95-0.73 (m, 6H) [note: piperidine protons not all visible]. LCMS (M+H)=567.32

### Example 122

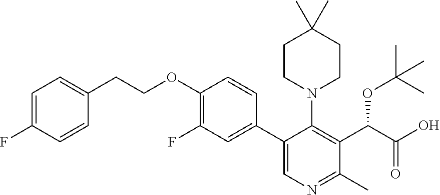 [SMILES: Cc1ncc(-c2ccc(OCCc3ccc(F)cc3)c(F)c2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(3-fluoro-4-(4-fluorophenethoxy)phenyl)-2-methylpyridin-3-yl)acetic acid

To a 7 mL vial charged with the entirety of isopropyl (S)-2-(tert-butoxy)-2-(4-(4,4-dimethylpiperidin-1-yl)-5-(3-fluoro-4-(4-fluorophenethoxy)phenyl)-2-methylpyridin-3-yl)acetate prepared above was added ethanol (4 mL) and a stir bar. To the solution was added aq. sodium hydroxide (5M, 0.511 mL, 2.56 mmol). The vial was sealed, then placed in a 90° C. heating block with stirring for 6 h. The reaction mixture was cooled to room temperature, then filtered through a 4 micron syringe filter. The filtrate was subjected to HPLC purification to afford (S)-2-(tert-butoxy)-2-(4-(4,4-dimethylpiperidin-1-yl)-5-(3-fluoro-4-(4-fluorophenethoxy)phenyl)-2-methylpyridin-3-yl)acetic acid as a white solid, 82 mg (66% over two steps). LCMS=567.30 (M+H). ¹H NMR (500 MHz, methanol-d4) δ 8.05 (s, 1H), 7.38-7.31 (m, 2H), 7.20 (t, J=8.5 Hz, 1H), 7.13 (br d, J=11.8 Hz, 1H), 7.07-6.99 (m, 3H), 5.72 (s, 1H), 4.31 (t, J=6.6 Hz, 2H), 3.12 (t, J=6.5 Hz, 2H), 3.12 (br s, 2H), 2.73 (br s, 2H), 2.63 (s, 3H), 1.52-1.34 (m, 4H), 1.18 (s, 9H), 0.89 (s, 6H).

### Example 123

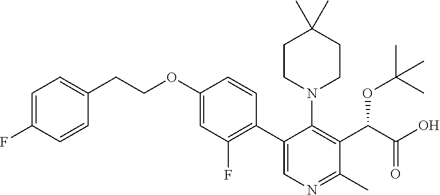 [SMILES: Cc1ncc(-c2ccc(OCCc3ccc(F)cc3)cc2F)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(2-fluoro-4-(4-fluorophenethoxy)phenyl)-2-methylpyridin-3-yl)acetic acid

To a 7 mL vial charged with the entirety of isopropyl (S)-2-(tert-butoxy)-2-(4-(4,4-dimethylpiperidin-1-yl)-5-(2-fluoro-4-(4-fluorophenethoxy)phenyl)-2-methylpyridin-3-yl)acetate prepared above was added ethanol (4 mL) and a stir bar. To the solution was added aq. sodium hydroxide (5M, 0.511 mL, 2.56 mmol). The vial was sealed, then placed in a 90° C. heating block with stirring for 4.5 h. The reaction mixture was cooled to room temperature, then was filtered through a 4 micron syringe filter. The filtrate was subjected to HPLC purification to afford (S)-2-(tert-butoxy)-2-(4-(4,4-dimethylpiperidin-1-yl)-5-(2-fluoro-4-(4-fluorophenethoxy)phenyl)-2-methylpyridin-3-yl)acetic acid as a white solid, 65 mg (52% over two steps). LCMS=567.30 (M+H). ¹H NMR (500 MHz, methanol-d4, two atropisomers exist in a 60:40 ratio, the major isomer is reported) δ 7.98-7.93 (m, 1H), 7.37-7.29 (m, 2H), 7.16 (br t, J=8.5 Hz, 1H), 7.07-6.98 (m, 1H), 6.89-6.83 (m, 1H), 6.83-6.79 (m, 1H), 5.74 (s, 1H), 4.25 (t, J=6.6 Hz, 2H), 3.10 (t, J=6.6 Hz, 2H), 2.61 (s, 3H), 1.49-1.31 (m, 4H), 1.17 (s, 9H), 0.84 (br s, 6H)

### Example 124

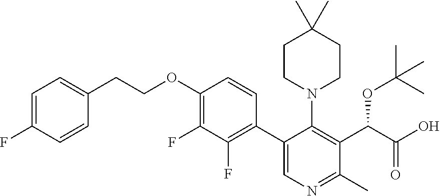 [SMILES: Cc1ncc(-c2ccc(OCCc3ccc(F)cc3)c(F)c2F)c(N2CCC(C)(C)CC2)c1[C@H](OC(C)(C)C)C(=O)O]

### (S)-2-(tert-Butoxy)-2-(5-(2,3-difluoro-4-(4-fluorophenethoxy)phenyl)-4-(4, 4-dimethylpiperidin-1-yl)-2-methylpyridin-3-yl)acetic acid

To a 7 mL vial charged with the entirety of (S)-isopropyl 2-(tert-butoxy)-2-(5-(2,3-difluoro-4-(4-fluorophenethoxy)phenyl)-4-(4,4-dimethylpiperidin-1-yl)-2-methylpyridin-3-yl)acetate prepared above was added ethanol (4 mL) and a stir bar. To the solution was added aq. sodium hydroxide (5M, 0.511 mL, 2.56 mmol). The vial was sealed, then placed in a 90° C. heating block with stirring for 4.5 h. The reaction mixture was cooled to room temperature, then was filtered through a 4 micron syringe filter. The filtrate was subjected to HPLC purification to afford (S)-2-(tert-butoxy)-2-(5-(2,3-difluoro-4-(4-fluorophenethoxy)phenyl)-4-(4,4-dimethylpiperidin-1-yl)-2-methylpyridin-3-yl)acetic acid as a white solid, 53 mg (41% over two steps). LCMS=585.25 (M+H). ¹H NMR (500 MHz, methanol-d4, two atropisomers exist in a 69:31 ratio, the major isomer is reported) δ 8.00-7.95 (m, 1H), 7.35 (dd, J=8.4, 5.4 Hz, 2H), 7.07-6.93 (m, 4H), 5.74 (br s, 1H), 4.33 (t, J=6.5 Hz, 2H), 3.13 (brt, J=6.4 Hz, 2H), 2.61 (s, 3H), 1.39 (br s, 4H), 1.17 (s, 9H), 0.84 (br s, 6H)

### Example 125

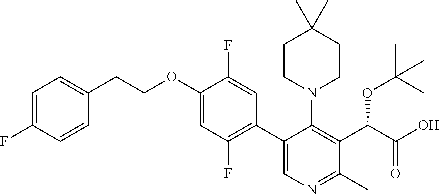 [SMILES: Cc1ncc(-c2cc(F)c(OCCc3ccc(F)cc3)cc2F)c(N2CCC(C)(C)CC2)c1[C@H](OC(C)(C)C)C(=O)O]

### (2S)-2-(tert-Butoxy)-2-(5-{2, 5-difluoro-4-[2-(4-fluorophenyl)ethoxy]phenyl}-4-(4, 4-dimethylpiperidin-1-yl)-2-methylpyridin-3-yl)acetic acid

To a 14 mL test tube equipped with a stir was added tribasic potassium phosphate (105 mg, 0.494 mmol), 2-(2,5-difluoro-4-(4-fluorophenethoxy)phenyl)-6-methyl-1,3,6,2-dioxazaborocane-4,8-dione (26.8 mg, 0.066 mmol), (S)-isopropyl 2-(5-bromo-4-(4,4-dimethylpiperidin-1-yl)-2-methylpyridin-3-yl)-2-(tert-butoxy)acetate (25 mg, 0.055 mmol), and SPhos-Pd-G3 (2.1 mg, 2.7 μmol). The test tube was sealed with a rubber septum and then placed under N₂ atmosphere. To the flask was added a degassed solution (N₂ sparging for 5 min.) of dioxane (0.563 mL) and water (0.188 mL). The test tube was placed in a 60° C. heating block with stirring for 24 h. The reaction mixture was cooled to r.t., then was diluted with Et₂O (5 mL) and then washed with water (5 mL). The organic phase was dried over MgSO₄, then filtered, then transferred to a 7 mL vial and evaporated under a N₂ stream. To the vial was added Ethanol (1.0 mL) and a stir bar. To the solution was added aq. sodium hydroxide (5M, 0.15 mL, 0.750 mmol). The vial was sealed, then placed in a 90° C. heating block with stirring for 3 h. The reaction mixture was cooled to r.t., then was filtered through a syringe filter. The crude material (filtrate) was purified via preparative LC/MS to afford (2S)-2-(tert-butoxy)-2-(5-{2,5-difluoro-4-[2-(4-fluorophenyl)ethoxy]phenyl}-4-(4,4-dimethylpiperidin-1-yl)-2-methylpyridin-3-yl)acetic acid (11.9 mg, 37%). LCMS (M+H):585.26. Ratio of atropisomers is 63:37 as determined by 1H NMR (500 MHz, methanol-d4) δ 5.76 (br s, 1H), 5.70 (br s, 0.6H). ¹HNMR (500 MHz, methanol-d4) δ 8.05-7.97 (m, 1H), 7.38 (dd, J=8.2, 5.8 Hz, 2H), 7.11-7.02 (m, 4H), 5.76 (br s, 0.63H), 5.70 (br s, 0.36H), 4.36-4.28 (m, 2H), 3.15 (brt, J=6.4 Hz, 2H), 2.95-2.69 (m, 2H), 2.68 (s, 3H), 2.00 (s, 3H), 1.43 (br s, 4H), 1.19 (s, 9H), 0.89 (br s, 6H).

### Example 126

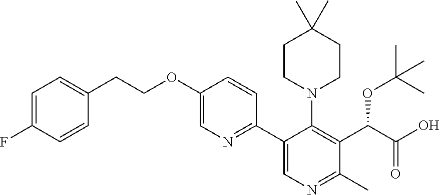 [SMILES: Cc1ncc(-c2ccc(OCCc3ccc(F)cc3)cn2)c(N2CCC(C)(C)CC2)c1[C@H](OC(C)(C)C)C(=O)O]

### (2S)-2-(tert-Butoxy)-2-[4′-(4, 4-dimethylpiperidin-1-yl)-5-[2-(4-fluorophenyl)ethoxy]-6′-methyl-[2, 3′-bipyridine]-5′-yl]acetic acid

To a 14 mL test tube equipped with a stir bar was added 2-(5-(4-fluorophenethoxy)pyridin-2-yl)-6-methyl-1,3,6,2-dioxazaborocane-4,8-dione (31 mg, 0.082 mmol), (S)-isopropyl 2-(5-bromo-4-(4,4-dimethylpiperidin-1-yl)-2-methylpyridin-3-yl)-2-(tert-butoxy)acetate (25 mg, 0.055 mmol), SPhos-Pd-G3 (2 mg, 3 μmol), diacetoxycopper (5 mg, 0.03 mmol) and anhydrous tribasic potassium phosphate (finely ground, 58.3 mg, 0.274 mmol). The test tube was sealed with a rubber septum, then placed under N₂ atmosphere. To the test tube was added a degassed (N₂ sparging for 5 min) solution of DMF and diethanolamine (5.8 mg, 0.055 mmol). The test tube was placed in a 100° C. heating block with stirring for 4 days (time not optimized). The reaction solution was cooled to r.t., then diluted with EtOAc (5 mL) and water (5 mL). The phases were mixed, then the aq. phase was discarded. The organic phase was dried over MgSO₄, then filtered into a 7 mL vial, then the volatiles were evaporated under a N2 stream. To the vial was added a stir bar and ethanol (1 mL), then aq. sodium hydroxide (5M, 0.11 mL, 0.55 mmol). The vial was placed in a 90° C. heating block with stirring for 3 h. The reaction was filtered through a syringe filter and to afford a solution of the crude product. The crude material was purified via preparative LC/MS to afford (2S)-2-(tert-butoxy)-2-[4′-(4,4-dimethylpiperidin-1-yl)-5-[2-(4-fluorophenyl)ethoxy]-6′-methyl-[2,3′-bipyridine]-5′-yl]acetic acid (3 mg, 10%). LCMS (M+H)=550.24. ¹H NMR (500 MHz, methanol-d4) δ 8.33 (d, J=2.4 Hz, 1H), 8.12 (s, 1H), 7.50 (dd, J=8.5, 2.7 Hz, 1H), 7.41 (d, J=8.5 Hz, 1H), 7.35-7.29 (m, 2H), 7.02 (br t, J=8.7 Hz, 2H), 5.88 (s, 1H), 4.35 (br t, J=6.6 Hz, 2H), 3.12 (br t, J=6.6 Hz, 2H), 2.93 (br s, 2H), 2.80 (br s, 2H), 2.63 (s, 3H), 1.41 (br s, 4H), 1.19 (s, 9H), 0.87 (s, 6H).

Examples 127 to 199 were synthesized by following one the general methods A-D described below.

### General Methods:

Method A: To a 7 mL vial equipped with a stir bar was added the amine (0.091 mmol), then a solution of aldehyde (0.046 mmol) and acetic acid (6.55 μl, 0.114 mmol) in dichloromethane (0.5 mL). To the solution was added methanol (0.25 mL), then sodium triacetoxyborohydride (19.4 mg, 0.091 mmol). The vial was capped and the solution was stirred at room temperature for 5-18 h. The reaction solution was concentrated under a nitrogen gas stream, and the resulting residue was dissolved in a EtOAc (4 mL) and was transferred to a 14 mL test tube. The mixture was washed with aq. NaOH (1M, 2 mL). The aq. phase was back-extracted with EtOAc (1 mL). The combined organics were washed with brine (2 mL), then dried over MgSO₄, then filtered into a 7 mL vial. The volatiles were removed under a nitrogen stream. To the vial was added a stir bar and ethanol (1 mL), then aq. NaOH (5M, 0.091 mL, 0.457 mmol). The vial was capped, then placed in a 80° C. heating block with stirring for 24 h. Reaction progress was monitored by LCMS. If conversion was less than 50%, to the reaction solution was added aq. NaOH (5M, 0.091 mL, 0.457 mmol) and the resulting mixture was stirred at 80° C. for 24 h. The mixture was filtered through a syringe filter to afford a solution of the crude product.
Method B: To a 14 mL test tube equipped with a stir bar was added (S)-isopropyl 2-(6-(7-azaspiro[3.5]nonan-7-ylmethyl)-5-bromo-4-(4,4-dimethylpiperidin-1-yl)-2-methylpyridin-3-yl)-2-(tert-butoxy)acetate (28.9 mg, 0.049 mmol), the boronic acid (0.098 mmol), bis(di-tert-butyl(4-dimethylaminophenyl)phosphine)dichloropalladium(II) (3.45 mg, 4.88 μmol) and tribasic potassium phosphate (62.1 mg, 0.293 mmol). The test tube was sealed with a rubber septum, then placed under nitrogen atmosphere. To the test tube was added a degassed (nitrogen sparging for 5 min) dioxane (0.75 mL)+water (0.25 mL). The test tube was placed in a 90° C. heating block with stirring for 3-18 h. The reaction mixture was diluted with Et₂O (5 mL) and then was washed with water (5 mL). The organic phase was dried over MgSO₄, then filtered, then was concentrated in a 7 mL vial under a nitrogen stream. To the vial was added a stir bar and EtOH (1.0 mL), then aq. sodium hydroxide (5M, 0.098 mL, 0.488 mmol). The vial was capped, then placed in a 90° C. heating block with stirring for 5-18 h. The reaction mixture was filtered through a syringe filter to afford a solution of the crude product.
Method C: To a 14 mL test tube equipped with a stir and charged with the bromide (0.055 mmol) and (2-Dicyclohexylphosphino-2′,6′-dimethoxybiphenyl) [2-(2′-amino-1,1′-biphenyl)]palladium(II) methanesulfonate (“SPhos-Pd-G3”, 2.1 mg, 2.7 μmol) was added tribasic potassium phosphate (105 mg, 0.494 mmol) and the boronic acid (0.082 mmol). The flask was sealed with a rubber septum, then was placed under nitrogen atmosphere. To the test tube was added a degassed (nitrogen sparging for 5 min) solution of dioxane (0.563 mL) and water (0.188 mL). The test tube was placed in a 80° C. heating block with stirring for 2-18 h. The reaction mixture was diluted with diethyl ether (5 mL) and then washed with water (5 mL). The organic phase was dried over MgSO₄, then filtered, then transferred to a 7 mL vial where the volatiles were evaporated under a nitrogen gas stream. To the vial was added ethanol (1.0 mL) and a stir bar. To the solution was added aq. sodium hydroxide (5M, 0.15 mL, 0.75 mmol). The vial was sealed, then placed in a 90° C. heating block with stirring for 2-18 h. The reaction mixture was filtered to afford a solution of the crude product.
Method D: To a 14 mL test tube equipped with a stir bar was added the boronate (0.082 mmol), the bromide (0.055 mmol), Pd(PPh₃)₄(13 mg, 11 μmol), Cu(OAc)₂ (5 mg, 0.03 mmol) and anhydrous tribasic potassium phosphate (finely ground, 58.3 mg, 0.274 mmol). The test tube was sealed with a rubber septum, then placed under N₂ atmosphere. To the test tube was added a degassed (N₂ sparging for 5 min) solution of DMF (0.5 mL) and diethanolamine (6 mg, 0.06 mmol). The test tube was placed in a 85° C. heating block with stirring for 18 h. The reaction mixture was cooled to r.t., then was diluted with EtOAc (5 mL) and water (5 mL). The isolated organic phase was dried over MgSO₄, then filtered, then concentrated in a 7 mL vial under a N₂ stream. To the vial was added a stir bar and ethanol (1.0 mL), then aq. sodium hydroxide (5M, 1.1 mL, 0.55 mmol). The vial was sealed with a cap, then placed in a 90° C. heating block with stirring for 3-18 h. The mixture reaction mixture was cooled to room temperature, then filtered through a syringe filter to afford a solution of the crude product.

### Example 127

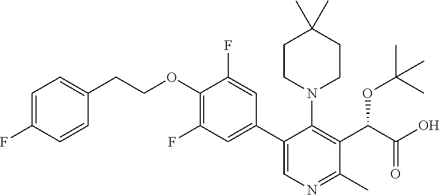 [SMILES: Cc1ncc(-c2cc(F)c(OCCc3ccc(F)cc3)c(F)c2)c(N2CCC(C)(C)CC2)c1[C@H](OC(C)(C)C)C(=O)O]

### (S)-2-(tert-Butoxy)-2-(5-(3, 5-difluoro-4-(4-fluorophenethoxy)phenyl)-4-(4, 4-dimethylpiperidin-1-yl)-2-methylpyridin-3-yl)acetic acid

General method C was followed on a 55 μmol reaction scale using 2-(3,5-difluoro-4-(4-fluorophenethoxy)phenyl)-4,4,5,5-tetramethyl-1,3,2-dioxaborolane and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (S)-2-(tert-butoxy)-2-(5-(3,5-difluoro-4-(4-fluorophenethoxy)phenyl)-4-(4,4-dimethylpiperidin-1-yl)-2-methylpyridin-3-yl)acetic acid (9.6 mg, 30%). LCMS (M+H)=585.2. ¹H NMR (500 MHz, methanol-d4) δ 8.04 (s, 1H), 7.30 (dd, J=8.5, 5.5 Hz, 2H), 7.04-6.94 (m, 4H), 5.86 (s, 1H), 4.40 (t, J=6.7 Hz, 2H), 3.08 (t, J=6.7 Hz, 2H), 3.03 (br s, 1H), 2.74 (br s, 2H), 2.61 (s, 3H), 1.48-1.36 (m, 4H), 1.19 (s, 9H), 0.89 (s, 6H).

### Example 128

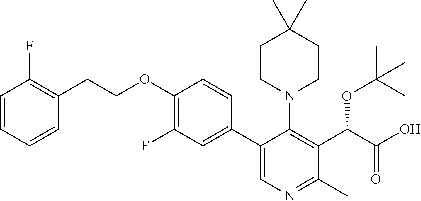 [SMILES: Cc1ncc(-c2ccc(OCCc3ccccc3F)c(F)c2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(3-fluoro-4-(2-fluorophenethoxy)phenyl)-2-methylpyridin-3-yl)acetic acid

General method C was followed on a 55 μmol reaction scale (3-fluoro-4-(2-fluorophenethoxy)phenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (S)-2-(tert-butoxy)-2-(4-(4,4-dimethylpiperidin-1-yl)-5-(3-fluoro-4-(2-fluorophenethoxy)phenyl)-2-methylpyridin-3-yl)acetic acid (12.9 mg, 42%). LCMS (M+H)=567.2. ¹H NMR (500 MHz, methanol-d4) δ 8.05 (s, 1H), 7.42-7.36 (m, 1H), 7.31-7.24 (m, 1H), 7.20 (t, J=8.5 Hz, 1H), 7.15-7.09 (m, 2H), 7.06 (dd, J=8.1, 6.0 Hz, 2H), 5.85 (s, 1H), 4.37 (t, J=6.9 Hz, 2H), 3.19 (t, J=6.7 Hz, 2H), 3.14-3.03 (m, 2H), 2.75 (br dd, J=11.0, 6.1 Hz, 2H), 2.64 (s, 3H), 1.50-1.37 (m, 4H), 1.21 (s, 9H), 0.89 (s, 6H).

### Example 129

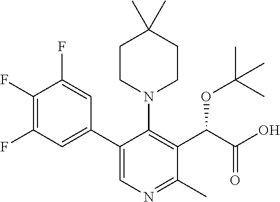 [SMILES: Cc1ncc(-c2cc(F)c(F)c(F)c2)c(N2CCC(C)(C)CC2)c1[C@H](OC(C)(C)C)C(=O)O]

### (2S)-2-(tert-Butoxy)-2-[4-(4, 4-dimethylpiperidin-1-yl)-2-methyl-5-(3,4, 5-trifluorophenyl)pyridin-3-yl]acetic acid

General method C was followed where the coupling was performed at 60 deg C. for 3 h. The reaction scale was 55 μmol and the coupling partners were (3,4,5-trifluorophenyl)boronic acid and (S)-isopropyl 2-(5-bromo-4-(4,4-dimethylpiperidin-1-yl)-2-methylpyridin-3-yl)-2-(tert-butoxy)acetate. The crude material was purified via preparative LC/MS to afford (2S)-2-(tert-butoxy)-2-[4-(4,4-dimethylpiperidin-1-yl)-2-methyl-5-(3,4,5-trifluorophenyl)pyridin-3-yl]acetic acid (11.5 mg, 45%). LCMS (M+H)+465.21. ¹H NMR (500 MHz, methanol-d4) δ 8.13 (s, 1H), 7.26-7.21 (m, 2H), 5.80 (s, 1H), 2.88-2.70 (m, 2H), 2.64 (s, 3H), 1.47 (br s, 4H), 1.21 (s, 9H), 0.94 (s, 6H).

### Example 130

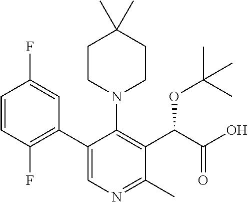 [SMILES: Cc1ncc(-c2cc(F)ccc2F)c(N2CCC(C)(C)CC2)c1[C@H](OC(C)(C)C)C(=O)O]

### (2S)-2-(tert-Butoxy)-2-[5-(2, 5-difluorophenyl)-4-(4, 4-dimethylpiperidin-1-yl)-2-methylpyridin-3-yl]acetic acid

General method C was followed where the coupling was performed at 60 deg C. for 3 h. The reaction scale was 55 μmol and the coupling partners were (2,5-difluorophenyl)boronic acid and (S)-isopropyl 2-(5-bromo-4-(4,4-dimethylpiperidin-1-yl)-2-methylpyridin-3-yl)-2-(tert-butoxy)acetate. The crude material was purified via preparative LC/MS to afford (2S)-2-(tert-butoxy)-2-[5-(2,5-difluorophenyl)-4-(4,4-dimethylpiperidin-1-yl)-2-methylpyridin-3-yl]acetic acid (11.7 mg, 48%). LCMS (M+H)=447.2. Ratio of atropisomers is 54:46 as determined by ¹H NMR (500 MHz, methanol-d4) δ 5.85 (br s, 1.00H), 5.76 (br s, 0.84H).

¹H NMR (500 MHz, methanol-d4) δ 8.12 (br s, 0.6H), 8.07 (br s, 0.4H), 7.37-7.22 (m, 2.8H), 7.16-7.09 (m, 0.6H), 5.85 (br s, 0.5H), 5.76 (br s, 0.4H), 3.10 (br s, 1.6H), 2.91 (br s, 1H), 2.78 (br s, 1.2H), 2.65 (s, 3H), 1.44 (br s, 4H), 1.22 (s, 9H), 0.90 (br d, J=12.8 Hz, 6H).

### Example 131

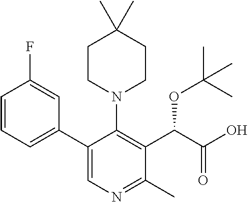 [SMILES: Cc1ncc(-c2cccc(F)c2)c(N2CCC(C)(C)CC2)c1[C@H](OC(C)(C)C)C(=O)O]

### (2S)-2-(tert-Butoxy)-2-[4-(4, 4-dimethylpiperidin-1-yl)-5-(2-fluorophenyl)-2-methylpyridin-3-yl]acetic acid

General method C, where the coupling was performed at 60 deg C. for 3 h, was followed on a 55 μmol reaction scale using (2-fluorophenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-5-(2-fluorophenyl)-2-methylpyridin-3-yl]acetic acid (13.5 mg, 57%). LCMS (M+H)=429.2. Ratio of atropisomers is 58:42 as determined by 1H NMR (500 MHz, methanol-d4) δ 5.82 (s, 1H), 5.73 (s, 0.72H). 1H NMR (500 MHz, methanol-d4) δ 8.11 (s, 0.6H), 8.05 (s, 0.4H), 7.54 (br d, J=4.9 Hz, 1H), 7.46-7.39 (m, 0.4H), 7.38-7.24 (m, 2.8H), 5.82 (s, 0.6H), 5.73 (s, 0.4H), 3.27-2.97 (m, 1.6H), 2.96-2.83 (m, 1H), 2.82-2.69 (m, 1.2H), 2.67 (s, 3H), 1.42 (br s, 4H), 1.22 (br d, J=5.8 Hz, 9H), 0.91 (s, 2.4H), 0.88-0.84 (m, 3.5H).

### Example 132

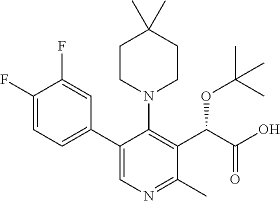 [SMILES: Cc1ncc(-c2ccc(F)c(F)c2)c(N2CCC(C)(C)CC2)c1[C@H](OC(C)(C)C)C(=O)O]

### (2S)-2-(tert-Butoxy)-2-[5-(2, 3-difluorophenyl)-4-(4, 4-dimethylpiperidin-1-yl)-2-methylpyridin-3-yl]acetic acid

General method C, where the coupling was performed at 60 deg C. for 3 h, was followed on a 55 μmol reaction scale using (2,3-difluorophenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[5-(2,3-difluorophenyl)-4-(4,4-dimethylpiperidin-1-yl)-2-methylpyridin-3-yl]acetic acid (4.7 mg, 15%). LCMS (M+H)=447.2. Ratio of atropisomers is 61:39 as determined by 1H NMR (500 MHz, methanol-d4) δ 5.84 (s, 1H), 5.77 (s, 0.62H).

1H NMR (500 MHz, methanol-d4) δ 8.11 (s, 0.61H), 8.05 (s, 0.39H), 7.46-7.38 (m, 1H), 7.36-7.29 (m, 1H), 7.22 (br t, J=6.6 Hz, 0.39H), 7.13 (br t, J=6.6 Hz, 0.61H), 5.84 (s, 0.61H), 5.77 (s, 0.39H), 3.09 (br s, 1.56H), 2.87 (br s, 1H), 2.75 (br s, 1.24H), 2.65 (s, 3H), 2.01 (s, 0.21H), 1.43 (br s, 4H), 1.22-1.20 (m, 9H), 0.94-0.89 (m, 2.3H), 0.89-0.85 (m, 3.6H).

### Example 133

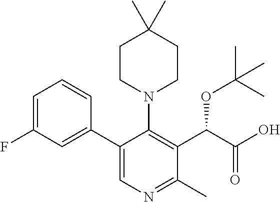 [SMILES: Cc1ncc(-c2cccc(F)c2)c(N2CCC(C)(C)CC2)c1[C@H](OC(C)(C)C)C(=O)O]

### (2S)-2-(tert-Butoxy)-2-[4-(4, 4-dimethylpiperidin-1-yl)-5-(3-fluorophenyl)-2-methylpyridin-3-yl]acetic acid

General method C, where the coupling was performed at 60 deg C. for 3 h, was followed on a 55 μmol reaction scale using (3-fluorophenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-5-(3-fluorophenyl)-2-methylpyridin-3-yl]acetic acid (11 mg, 47%). LCMS (M+H)=429.2. ¹H NMR (500 MHz, methanol-d4) δ 8.09 (s, 1H), 7.56-7.50 (m, 1H), 7.26-7.13 (m, 3H), 5.74 (s, 1H), 2.75 (br s, 2H), 2.65 (s, 3H), 1.42 (br s, 4H), 1.19 (s, 9H), 0.88 (s, 6H).

### Example 134

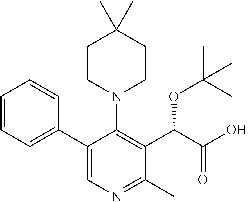 [SMILES: Cc1ncc(-c2ccccc2)c(N2CCC(C)(C)CC2)c1[C@H](OC(C)(C)C)C(=O)O]

### (2S)-2-(tert-Butoxy)-2-[4-(4, 4-dimethylpiperidin-1-yl)-2-methyl-5-phenylpyridin-3-yl]acetic acid

General method C was followed on a 55 μmol reaction scale using phenylboronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-2-methyl-5-phenylpyridin-3-yl]acetic acid (11.3 mg, 50%). LCMS (M+H)=411.13. ¹H NMR (500 MHz, DMSO-d6) δ 8.04 (s, 1H), 7.51-7.45 (m, 2H), 7.45-7.40 (m, 1H), 7.32 (d, J=7.3 Hz, 2H), 5.87 (s, 1H), 2.51 (s, 3H), 1.30 (br d, J=2.1 Hz, 2H), 1.14 (s, 9H), 0.87-0.70 (m, 6H).

### Example 135

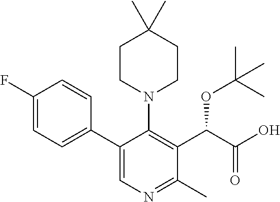 [SMILES: Cc1ncc(-c2ccc(F)cc2)c(N2CCC(C)(C)CC2)c1[C@H](OC(C)(C)C)C(=O)O]

### (2S)-2-(tert-Butoxy)-2-[4-(4, 4-dimethylpiperidin-1-yl)-5-(4-fluorophenyl)-2-methylpyridin-3-yl]acetic acid

General method C was followed on a 55 μmol reaction scale using (4-fluorophenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-5-(4-fluorophenyl)-2-methylpyridin-3-yl]acetic acid (10.1 mg, 43%). LCMS (M+H)=429.12. ¹H NMR (500 MHz, methanol-d4) δ 8.06 (s, 1H), 7.41-7.34 (m, 2H), 7.24 (t, J=8.7 Hz, 2H), 5.88 (s, 1H), 3.13-2.98 (m, 2H), 2.74 (br d, J=7.0 Hz, 2H), 2.65 (s, 3H), 1.48-1.36 (m, 4H), 1.22 (s, 9H), 0.89 (s, 6H).

### Example 136

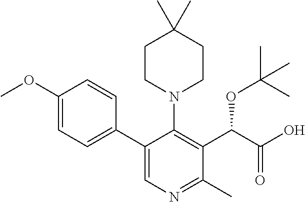 [SMILES: COc1ccc(-c2cnc(C)c([C@H](OC(C)(C)C)C(=O)O)c2N2CCC(C)(C)CC2)cc1]

### (2S)-2-(tert-Butoxy)-2-[4-(4, 4-dimethylpiperidin-1-yl)-5-(4-methoxyphenyl)-2-methylpyridin-3-yl]acetic acid

General method C was followed on a 55 μmol reaction scale using (4-methoxyphenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-5-(4-methoxyphenyl)-2-methylpyridin-3-yl]acetic acid (11.3 mg, 47%). LCMS (M+H)=441.14. ¹H NMR (500 MHz, methanol-d4) δ 8.06 (s, 1H), 7.27 (d, J=8.5 Hz, 2H), 7.08 (d, J=8.5 Hz, 2H), 5.81 (s, 1H), 3.88 (s, 3H), 3.18-3.05 (m, 2H), 2.84-2.73 (m, 2H), 2.68 (s, 3H), 1.52-1.36 (m, 4H), 1.22 (s, 9H), 0.90 (s, 6H).

### Example 137

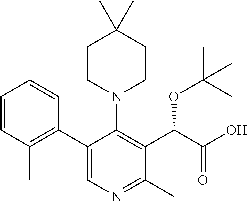 [SMILES: Cc1ccccc1-c1cnc(C)c([C@H](OC(C)(C)C)C(=O)O)c1N1CCC(C)(C)CC1]

### (2S)-2-(tert-Butoxy)-2-[4-(4, 4-dimethylpiperidin-1-yl)-2-methyl-5-(2-methylphenyl)pyridin-3-yl]acetic acid

General method C was followed on a 55 μmol reaction scale using 6-methyl-2-(o-tolyl)-1,3,6,2-dioxazaborocane-4,8-dione and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-2-methyl-5-(2-methylphenyl)pyridin-3-yl]acetic acid (3.3 mg, 14%). LCMS (M+H)=425.15. Ratio of atropisomers is 80:20 by ¹H NMR (500 MHz, methanol-d4) δ 6.71 (s, 1H), 6.66 (s, 0.24H); Ratio of atropisomers is 77:23 by HPLC.

¹H NMR (500 MHz, methanol-d4) δ 8.77 (s, 0.8H), 8.68 (s, 0.2H), 8.19-8.12 (m, 2.35H), 8.11-8.00 (m, 1H), 7.93 (d, J=7.3 Hz, 0.8H), 6.71 (s, 0.8H), 6.66 (s, 0.2H), 2.92 (s, 2.4H), 2.81 (s, 0.6H), 2.14-2.02 (m, 2.6H), 1.98-1.96 (m, 1.8H), 1.95 (s, 8.5H), 1.54 (br s, 6H)

### Example 138

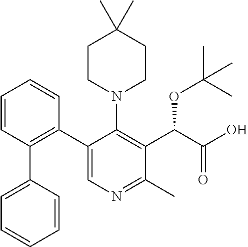 [SMILES: Cc1ncc(-c2ccccc2-c2ccccc2)c(N2CCC(C)(C)CC2)c1[C@H](OC(C)(C)C)C(=O)O]

### (2S)-2-(5-{[1,1′-Biphenyl]-2-yl}-4-(4, 4-dimethylpiperidin-1-yl)-2-methylpyridin-3-yl)-2-(tert-butoxy)acetic acid

General method C was followed on a 55 μmol reaction scale using [1,1′-biphenyl]-2-ylboronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(5-{[1,1′-biphenyl]-2-yl}-4-(4,4-dimethylpiperidin-1-yl)-2-methylpyridin-3-yl)-2-(tert-butoxy)acetic acid (14.3 mg, 53%). LCMS (M+H)=487.19, 487.19. Ratio of atropisomers is 75:25 as determined by ¹H NMR (500 MHz, methanol-d4) δ 6.66 (s, 1H), 6.27 (s, 0.34H); Ratio of atropisomers is 60:40 as determined by HPLC.

¹H NMR (500 MHz, methanol-d4) δ 8.91 (s, 0.25H), 8.46 (s, 0.75H), 8.37-8.28 (m, 3H), 8.13 (br d, J=7.3 Hz, 1H), 8.07-8.00 (m, 3H), 7.99-7.91 (m, 2.25H), 6.66 (s, 0.75H), 6.27 (s, 0.25H), 3.29 (br s, 0.75H), 3.19 (s, 2.25H), 2.21-2.05 (m, 2H), 1.89 (s, 6.75H), 1.87 (s, 2.3H), 1.73-1.52 (m, 6H).

### Example 139

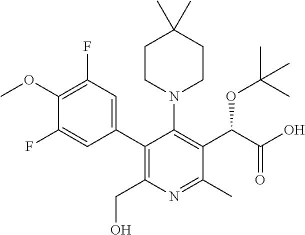 [SMILES: COc1c(F)cc(-c2c(CO)nc(C)c([C@H](OC(C)(C)C)C(=O)O)c2N2CCC(C)(C)CC2)cc1F]

### (S)-2-(tert-Butoxy)-2-(5-(3, 5-difluoro-4-methoxyphenyl)-4-(4, 4-dimethylpiperidin-1-yl)-6-(hydroxymethyl)-2-methylpyridin-3-yl)acetic acid

To a 7 mL vial equipped with a stir bar and charged with (S)-isopropyl 2-(tert-butoxy)-2-(5-(3,5-difluoro-4-methoxyphenyl)-4-(4,4-dimethylpiperidin-1-yl)-6-(hydroxymethyl)-2-methylpyridin-3-yl)acetate (10 mg, 0.018 mmol) was added ethanol (0.50 mL) and aq. sodium hydroxide (5M, 0.050 mL, 0.250 mmol). The vial was placed in a 90° C. heating block with stirring for 7 h. The reaction mixture was filtered through a syringe filter to afford a solution of the crude product. The crude material was purified via preparative LC/MS to afford (S)-2-(tert-butoxy)-2-(5-(3,5-difluoro-4-methoxyphenyl)-4-(4,4-dimethylpiperidin-1-yl)-6-(hydroxymethyl)-2-methylpyridin-3-yl)acetic acid (7.0 mg, 0.014 mmol, 76% yield). LCMS (M+H)=507.21. ¹H NMR (500 MHz, methanol-d4) δ 7.12 (br d, J=10.6 Hz, 1H), 6.92 (br d, J=10.6 Hz, 1H), 5.84 (s, 1H), 4.47-4.41 (m, 1H), 4.29 (d, J=13.9 Hz, 1H), 4.06 (s, 3H), 3.03-2.84 (m, 2H), 2.83-2.73 (m, 2H), 2.70 (s, 3H), 2.67 (s, 1H), 1.39 (br s, 4H), 1.22 (s, 9H), 0.87 (s, 6H).

### Example 140

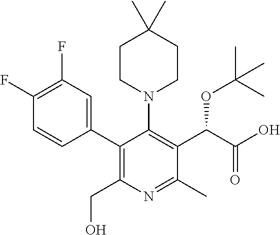 [SMILES: Cc1nc(CO)c(-c2ccc(F)c(F)c2)c(N2CCC(C)(C)CC2)c1[C@H](OC(C)(C)C)C(=O)O]

### (2S)-2-(tert-Butoxy)-2-[5-(3, 4-difluorophenyl)-4-(4, 4-dimethylpiperidin-1-yl)-6-(hydroxymethyl)-2-methylpyridin-3-yl]acetic acid

To a 7 mL vial equipped with a stir bar and charged with (S)-isopropyl 2-(tert-butoxy)-2-(5-(3,4-difluorophenyl)-4-(4,4-dimethylpiperidin-1-yl)-6-(hydroxymethyl)-2-methylpyridin-3-yl)acetate (10 mg, 0.019 mmol) was added ethanol (0.50 mL) and aq. sodium hydroxide (5M, 0.050 mL, 0.250 mmol). The vial was placed in a 90° C. heating block with stirring for 7 h. The reaction mixture was filtered through a syringe filter to afford a solution of the crude product. The crude material was purified via preparative LC/MS to afford (2S)-2-(tert-butoxy)-2-[5-(3,4-difluorophenyl)-4-(4,4-dimethylpiperidin-1-yl)-6-(hydroxymethyl)-2-methylpyridin-3-yl]acetic acid (6 mg, 66%). LCMS (M+H)=477.15. Ratio of atropisomers is 1.0:1.0 as determined by ¹H NMR (500 MHz, methanol-d4) δ 7.26-7.22 (m, 1H), 7.08-7.02 (m, 1H).

¹H NMR (500 MHz, methanol-d4) δ 7.50-7.36 (m, 1.5H), 7.24 (br dd, J=8.1, 2.6 Hz, 0.5H), 7.22-7.17 (m, 0.5H), 7.05 (dt, J=3.9, 2.1 Hz, 0.5H), 5.83 (s, 1H), 4.46-4.40 (m, 0.5H), 4.26 (d, J=14.3 Hz, 1H), 3.02-2.84 (m, 1.5H), 2.83-2.74 (m, 2H), 2.72 (s, 3H), 2.67 (s, 0.5H), 1.42-1.34 (m, 4H), 1.23 (s, 9H), 0.85 (d, J=4.0 Hz, 6H).

### Example 141

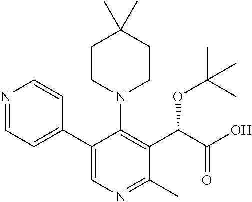 [SMILES: Cc1ncc(-c2ccncc2)c(N2CCC(C)(C)CC2)c1[C@H](OC(C)(C)C)C(=O)O]

### (2S)-2-(tert-Butoxy)-2-[4-(4, 4-dimethylpiperidin-1-yl)-6-methyl-[3, 4′-bipyridine]-5-yl]acetic acid

General method C was followed on a 55 μmol reaction scale using pyridin-4-ylboronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-6-methyl-[3,4′-bipyridine]-5-yl]acetic acid (0.7 mg, 3%). LCMS (M+H)=412.22. ¹H NMR (500 MHz, DMSO-d6) δ 7.85 (br s, 2H), 7.25 (br s, 1H), 6.65 (br s, 2H), 5.04 (br s, 1H), 2.28 (br s, 2H), 1.93 (br s, 2H), 1.84 (br s, 3H), 0.62 (br s, 4H), 0.40 (br s, 9H), 0.08 (br s, 6H).

### Example 142

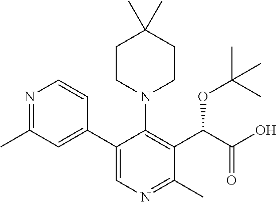 [SMILES: Cc1cc(-c2cnc(C)c([C@H](OC(C)(C)C)C(=O)O)c2N2CCC(C)(C)CC2)ccn1]

### (2S)-2-(tert-Butoxy)-2-[4-(4, 4-dimethylpiperidin-1-yl)-2′,6-dimethyl-[3, 4′-bipyridine]-5-yl]acetic acid

General method C was followed on a 55 μmol reaction scale using (2-methylpyridin-4-yl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-2′,6-dimethyl-[3,4′-bipyridine]-5-yl]acetic acid (4.4 mg, 19%). LCMS (M+H)=426.15. ¹H NMR (500 MHz, methanol-d4) δ 8.53 (d, J=5.1 Hz, 1H), 8.07 (s, 1H), 7.33 (s, 1H), 7.26 (d, J=4.8 Hz, 1H), 5.89 (s, 1H), 3.08 (br s, 2H), 2.75 (br s, 2H), 2.64 (s, 3H), 2.63 (s, 3H), 1.44 (br s, 4H), 1.21 (s, 9H), 0.90 (s, 6H).

### Example 143 Example 144

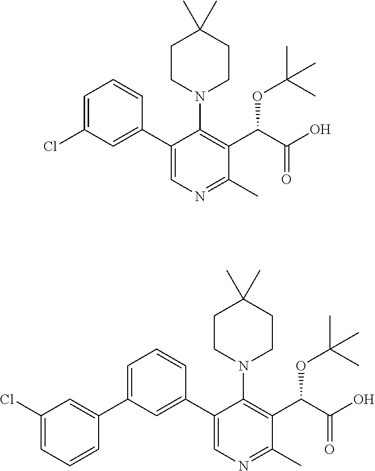 [SMILES: Cc1ncc(-c2cccc(Cl)c2)c(N2CCC(C)(C)CC2)c1[C@H](OC(C)(C)C)C(=O)O ; Cc1ncc(-c2cccc(-c3cccc(Cl)c3)c2)c(N2CCC(C)(C)CC2)c1[C@H](OC(C)(C)C)C(=O)O]

### (2S)-2-(tert-Butoxy)-2-[5-(3-chlorophenyl)-4-(4, 4-dimethylpiperidin-1-yl)-2-methylpyridin-3-yl]acetic acid and (2S)-2-(tert-butoxy)-2-(5-{3′-chloro-[1,1′-biphenyl]-3-yl}-4-(4, 4-dimethylpiperidin-1-yl)-2-methylpyridin-3-yl)acetic acid

The Suzuki cross-coupling procedure of general method C was followed on a 55 mol reaction scale using (3-chlorophenyl)boronic acid and (S)-isopropyl 2-(5-bromo-4-(4,4-dimethylpiperidin-1-yl)-2-methylpyridin-3-yl)-2-(tert-butoxy)acetate as starting material. Following the Suzuki cross-coupling reaction, the crude intermediate ester was further purified as follows: The crude material was dissolved in a min of acetone, then concentrated onto Celite in vacuo. The resulting powder was subjected to SiO₂ purification (24 g column, hexanes:EtOAc 100:0→60:40). Fractions were selected based on mass purity of the desired ester intermediate. Concentration of the pooled sample afforded a colorless solid, a mixture of isopropyl (S)-2-(tert-butoxy)-2-(5-(3-chlorophenyl)-4-(4,4-dimethylpiperidin-1-yl)-2-methylpyridin-3-yl)acetate and isopropyl (S)-2-(tert-butoxy)-2-(5-(3′-chloro-[1,1′-biphenyl]-3-yl)-4-(4,4-dimethylpiperidin-1-yl)-2-methylpyridin-3-yl)acetate. This material was transferred to a 7 mL vial. The saponification procedure of general method C was followed. The crude material was purified via preparative LC/MS to afford (2S)-2-(tert-butoxy)-2-[5-(3-chlorophenyl)-4-(4,4-dimethylpiperidin-1-yl)-2-methylpyridin-3-yl]acetic acid (4.9 mg, 20%) and (2S)-2-(tert-butoxy)-2-(5-{3′-chloro-[1,1′-biphenyl]-3-yl}-4-(4,4-dimethylpiperidin-1-yl)-2-methylpyridin-3-yl)acetic acid (2.1 mg, 7%).

### Analysis of Example 143:

LCMS (M+H)=445.11. ¹H NMR (500 MHz, DMSO-d6) δ 8.06 (s, 1H), 7.52-7.47 (m, 2H), 7.39 (s, 1H), 7.30 (br d, J=5.9 Hz, 1H), 5.77 (s, 1H), 2.48 (br s, 3H), 1.34-1.28 (br s, 2H), 1.15-1.12 (br s, 2H), 1.13 (s, 9H), 0.83 (br s, 6H).

### Analysis of Example 144:

LCMS (M+H)=521.16. H NMR (500 MHz, DMSO-d6) δ 8.11 (s, 1H), 7.79 (s, 1H), 7.75 (br d, J=7.7 Hz, 1H), 7.73-7.69 (m, 1H), 7.63-7.61 (m, 1H), 7.59 (t, J=7.7 Hz, 1H), 7.53-7.48 (m, 1H), 7.46-7.41 (m, 1H), 7.36 (br d, J=7.3 Hz, 1H), 5.77 (s, 1H), 2.55 (s, 3H), 1.92 (s, 0.65H), 1.29 (br dd, J=5.1, 1.5 Hz, 2H), 1.14 (s, 9H), 0.76 (br s, 6H).

### Example 145

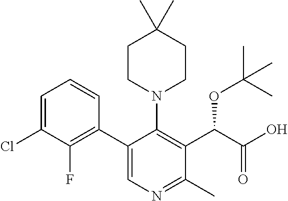 [SMILES: Cc1ncc(-c2cccc(Cl)c2F)c(N2CCC(C)(C)CC2)c1[C@H](OC(C)(C)C)C(=O)O]

### (2S)-2-(tert-Butoxy)-2-[5-(3-chloro-2-fluorophenyl)-4-(4, 4-dimethylpiperidin-1-yl)-2-methylpyridin-3-yl]acetic acid

The Suzuki cross-coupling procedure of general method C was followed on a 55 μmol reaction scale using (3-chloro-2-fluorophenyl)boronic acid and (S)-isopropyl 2-(5-bromo-4-(4,4-dimethylpiperidin-1-yl)-2-methylpyridin-3-yl)-2-(tert-butoxy)acetate as starting material. Following the Suzuki cross-coupling reaction, the crude intermediate ester was further purified as follows: The crude material was dissolved in a min of acetone, then concentrated onto Celite in vacuo. The resulting powder was subjected to SiO₂ purification (24 g column, hexanes:EtOAc 100:0460:40). Fractions were selected based on mass purity of the desired ester intermediate. Concentration of the pooled sample afforded a colorless solid, isopropyl (S)-2-(tert-butoxy)-2-(5-(3-chloro-2-fluorophenyl)-4-(4,4-dimethylpiperidin-1-yl)-2-methylpyridin-3-yl)acetate. This material was transferred to a 7 mL vial. The saponification procedure of general method C was followed. The crude material was purified via preparative LC/MS to afford (2S)-2-(tert-butoxy)-2-[5-(3-chloro-2-fluorophenyl)-4-(4,4-dimethylpiperidin-1-yl)-2-methylpyridin-3-yl]acetic acid (5.7 mg, 22%). LCMS (M+H)=463.11. Ratio of atropisomers is 1:1 as determined by ¹H NMR (500 MHz, DMSO-d6) δ 5.80 (s, 1H), 5.74 (s, 1H).

¹H NMR (500 MHz, DMSO-d6) δ 8.15-8.01 (m, 1H), 7.71-7.63 (m, 1H), 7.45-7.26 (m, 2H), 5.80 (s, 0.5H), 5.74 (s, 0.5H), 2.52 (br s, 3H), 1.47-1.20 (m, 4H), 1.14 (s, 9H), 0.81 (br s, 6H).

### Example 146 Example 147

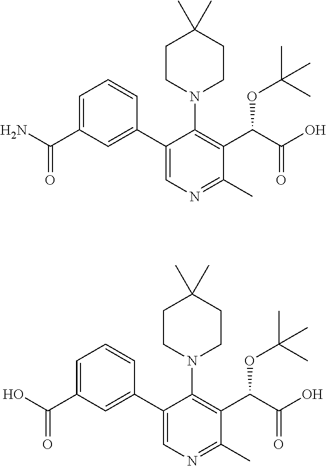 [SMILES: Cc1ncc(-c2cccc(C(N)=O)c2)c(N2CCC(C)(C)CC2)c1[C@H](OC(C)(C)C)C(=O)O ; Cc1ncc(-c2cccc(C(=O)O)c2)c(N2CCC(C)(C)CC2)c1[C@H](OC(C)(C)C)C(=O)O]

### (2S)-2-(tert-Butoxy)-2-[5-(3-carbamoylphenyl)-4-(4, 4-dimethylpiperidin-1-yl)-2-methylpyridin-3-yl]acetic acid and 3-{5-[(S)-(tert-butoxy) (carboxy)methyl]-4-(4, 4-dimethylpiperidin-1-yl)-6-methylpyridin-3-yl}benzoic acid

General method C was followed on a 55 μmol reaction scale using 3-(6-methyl-4,8-dioxo-1,3,6,2-dioxazaborocan-2-yl)benzonitrile and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[5-(3-carbamoylphenyl)-4-(4,4-dimethylpiperidin-1-yl)-2-methylpyridin-3-yl]acetic acid (4.9 mg, 20%) and 3-{5-[(S)-(tert-butoxy)(carboxy)methyl]-4-(4,4-dimethylpiperidin-1-yl)-6-methylpyridin-3-yl}benzoic acid (10.2 mg, 41%).

### Analysis of Example 146:

LCMS (M+H)=454.15. ¹H NMR (500 MHz, DMSO-d6) δ 8.08 (s, 1H), 7.96-7.91 (br s, 1H), 7.93 (br d, J=7.7 Hz, 1H), 7.85 (s, 1H), 7.59-7.52 (m, 1H), 7.48 (br d, J=7.7 Hz, 1H), 7.23 (s, 1H), 5.82 (s, 1H), 2.53 (s, 3H), 1.30 (br s, 2H), 1.15 (br s, 2H), 1.15 (s, 9H), 0.79 (br s, 6H).

### Analysis of Example 147:

LCMS (M+H)=455.13. ¹H NMR (500 MHz, DMSO-d6) δ 8.08 (s, 1H), 7.99 (br d, J=7.3 Hz, 1H), 7.86 (s, 1H), 7.62-7.53 (m, 2H), 5.82 (s, 1H), 2.53 (s, 3H), 1.30 (br d, J=5.1 Hz, 2H), 1.14 (s, 9H), 0.79 (br s, 6H).

### Example 148

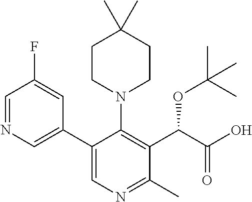 [SMILES: Cc1ncc(-c2cncc(F)c2)c(N2CCC(C)(C)CC2)c1[C@H](OC(C)(C)C)C(=O)O]

### (2S)-2-(tert-Butoxy)-2-[4-(4, 4-dimethylpiperidin-1-yl)-5′-fluoro-6-methyl-[3, 3′-bipyridine]-5-yl]acetic acid

General method C was followed on a 55 μmol reaction scale using (5-fluoropyridin-3-yl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-5′-fluoro-6-methyl-[3,3′-bipyridine]-5-yl]acetic acid (7.4 mg, 31%). LCMS (M+H)=430.1. ¹H NMR (500 MHz, DMSO-d6) δ 8.64 (d, J=2.6 Hz, 1H), 8.43 (s, 1H), 8.12 (s, 1H), 7.79 (br d, J=9.2 Hz, 1H), 5.81 (s, 1H), 2.54 (s, 3H), 1.32 (br s, 2H), 1.15 (s, 9H), 0.82 (br s, 6H).

### Example 149

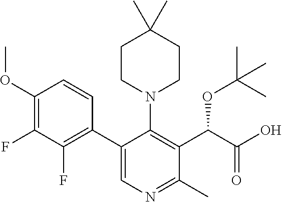 [SMILES: COc1ccc(-c2cnc(C)c([C@H](OC(C)(C)C)C(=O)O)c2N2CCC(C)(C)CC2)c(F)c1F]

### (2S)-2-(tert-Butoxy)-2-[5-(2, 3-difluoro-4-methoxyphenyl)-4-(4, 4-dimethylpiperidin-1-yl)-2-methylpyridin-3-yl]acetic acid

General method C was followed on a 55 μmol reaction scale using (2,3-difluoro-4-methoxyphenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[5-(2,3-difluoro-4-methoxyphenyl)-4-(4,4-dimethylpiperidin-1-yl)-2-methylpyridin-3-yl]acetic acid (13 mg, 50%). LCMS (M+H)=477.13. ¹H NMR (500 MHz, DMSO-d6) δ 8.04 (br s, 1H), 7.17-7.08 (m, 2H), 5.82 (br s, 1H), 3.94 (s, 3H), 2.50 (s, 3H), 1.92 (s, 0.6H), 1.38-1.23 (m, 4H), 1.14 (s, 9H), 0.82 (br s, 6H).

### Example 150

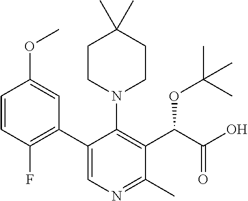 [SMILES: COc1ccc(F)c(-c2cnc(C)c([C@H](OC(C)(C)C)C(=O)O)c2N2CCC(C)(C)CC2)c1]

### (2S)-2-(tert-Butoxy)-2-[4-(4, 4-dimethylpiperidin-1-yl)-5-(2-fluoro-5-methoxyphenyl)-2-methylpyridin-3-yl]acetic acid

General method C was followed on a 55 μmol reaction scale using (2-fluoro-5-methoxyphenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-5-(2-fluoro-5-methoxyphenyl)-2-methylpyridin-3-yl]acetic acid (10 mg, 40%). LCMS (M+H)=459.15. ¹H NMR (500 MHz, DMSO-d6) δ 8.04 (br s, 1H), 7.27-7.20 (m, 1H), 7.07-6.98 (m, 1H), 6.94-6.79 (m, 1H), 5.83 (br d, J=3.7 Hz, 1H), 3.79 (s, 3H), 2.50 (s, 3H), 1.37-1.27 (m, 4H), 1.15 (s, 9H), 0.81 (br d, J=1.5 Hz, 6H).

### Example 151

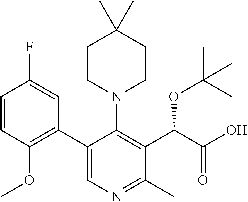 [SMILES: COc1ccc(F)cc1-c1cnc(C)c([C@H](OC(C)(C)C)C(=O)O)c1N1CCC(C)(C)CC1]

### (2S)-2-(tert-Butoxy)-2-[4-(4, 4-dimethylpiperidin-1-yl)-5-(5-fluoro-2-methoxyphenyl)-2-methylpyridin-3-yl]acetic acid

General method C was followed on a 55 μmol reaction scale using (5-fluoro-2-methoxyphenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-5-(5-fluoro-2-methoxyphenyl)-2-methylpyridin-3-yl]acetic acid (7.9 mg, 33%). LCMS (M+H)=459.15. Ratio of atropisomers is 55:45 as determined by ¹H NMR (500 MHz, DMSO-d6) δ 5.84 (br s, 1H), 5.78 (br d, J=0.7 Hz, 0.8H). ¹H NMR (500 MHz, DMSO-d6) δ 7.95 (br s, 0.55H), 7.89 (br s, 0.45H), 7.29-7.19 (m, 1H), 7.17-7.10 (m, 0.55H), 7.07 (br d, J=4.8 Hz, 1H), 7.00-6.94 (m, 0.45H), 5.84 (br s, 0.55H), 5.78 (br d, J=0.7 Hz, 0.45H), 3.71 (br d, J=6.6 Hz, 3H), 2.50 (br s, 3H), 1.29 (br d, J=4.4 Hz, 2H), 1.15 (br d, J=8.4 Hz, 9H), 0.81 (br s, 6H).

### Example 152

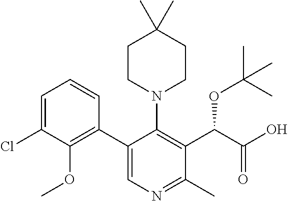 [SMILES: COc1c(Cl)cccc1-c1cnc(C)c([C@H](OC(C)(C)C)C(=O)O)c1N1CCC(C)(C)CC1]

### (2S)-2-(tert-Butoxy)-2-[5-(3-chloro-2-methoxyphenyl)-4-(4, 4-dimethylpiperidin-1-yl)-2-methylpyridin-3-yl]acetic acid

The Suzuki cross-coupling procedure of general method C was followed on a 55 mol reaction scale using (3-chloro-2-methoxyphenyl)boronic acid and (S)-isopropyl 2-(5-bromo-4-(4,4-dimethylpiperidin-1-yl)-2-methylpyridin-3-yl)-2-(tert-butoxy)acetate as starting material. The crude ester product was purified as follows: The crude material was dissolved in a min of acetone, then concentrated onto Celite in vacuo. The resulting powder was subjected to SiO₂ (24 g column, hexanes:EtOAc 100:0460:40). Fractions were selected based on mass purity of the desired ester intermediate. Concentration of the pooled sample afforded isopropyl (S)-2-(tert-butoxy)-2-(5-(3-chloro-2-methoxyphenyl)-4-(4,4-dimethylpiperidin-1-yl)-2-methylpyridin-3-yl)acetate as a colorless solid. The material was transferred to a 7 mL vial and the saponification procedure of general method C was followed. The crude material was purified via preparative LC/MS to afford (2S)-2-(tert-butoxy)-2-[5-(3-chloro-2-methoxyphenyl)-4-(4,4-dimethylpiperidin-1-yl)-2-methylpyridin-3-yl]acetic acid (4.7 mg, 18%). LCMS (M+H)=475.1. Ratio of atropisomers is 60:40 as determined by ¹H NMR (500 MHz, DMSO-d6) δ 8.05-8.02 (m, 1H), 7.99 (s, 0.7H).

¹H NMR (500 MHz, DMSO-d6) δ 8.04 (s, 0.6H), 7.99 (s, 0.4H), 7.54 (br d, J=8.1 Hz, 1H), 7.27-7.20 (m, 1.5H), 7.18-7.13 (m, 0.8H), 5.70-5.62 (m, 1H), 3.64 (s, 1H), 3.26 (br s, 1.8H), 2.49-2.46 (m, 3H), 1.91 (s, 0.7H), 1.34-1.19 (m, 3H), 1.11 (s, 9H), 0.80 (br s, 6H).

### Example 153 Example 154

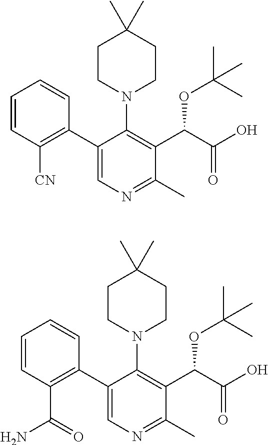

### (2S)-2-(tert-Butoxy)-2-[5-(2-cyanophenyl)-4-(4, 4-dimethylpiperidin-1-yl)-2-methylpyridin-3-yl]acetic acid and (2S)-2-(tert-butoxy)-2-[5-(2-carbamoylphenyl)-4-(4, 4-dimethylpiperidin-1-yl)-2-methylpyridin-3-yl]acetic acid

General method C was followed on a 55 μmol reaction scale using 2-(6-methyl-4,8-dioxo-1,3,6,2-dioxazaborocan-2-yl)benzonitrile and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[5-(2-cyanophenyl)-4-(4,4-dimethylpiperidin-1-yl)-2-methylpyridin-3-yl]acetic acid (1.7 mg, 7%) and (2S)-2-(tert-butoxy)-2-[5-(2-carbamoylphenyl)-4-(4,4-dimethylpiperidin-1-yl)-2-methylpyridin-3-yl]acetic acid (1 mg, 4%).

### Analysis of Example 153:

LCMS (M+H)=436.13. Ratio of atropisomers is 75:25 as determined by ¹H NMR (500 MHz, DMSO-d6) δ 5.80 (s, 1H), 5.73 (s, 0.33H).

¹H NMR (500 MHz, DMSO-d6) δ 8.07 (s, 0.75H), 8.01-7.91 (m, 1.25H), 7.85-7.76 (m, 1.25H), 7.64 (br t, J=7.5 Hz, 1.25H), 7.59 (br d, J=8.1 Hz, 0.25H), 7.50 (d, J=7.7 Hz, 0.75H), 5.80 (s, 0.75H), 5.73 (s, 0.25H), 2.54 (s, 3H), 1.91 (s, 0.6H), 1.33-1.22 (m, 3H), 1.15-1.12 (m, 9H), 0.76 (br s, 6H).

### Analysis of Example 154:

LCMS (M+H)=454.15. Ratio of atropisomers is 66:34 as determined by ¹H NMR (500 MHz, DMSO-d6) δ 5.73 (s, 1H), 5.37 (s, 0.5H).

¹H NMR (500 MHz, DMSO-d6) δ 8.09 (s, 0.25H), 8.05 (s, 0.75H), 7.65 (br d, J=7.3 Hz, 0.8H), 7.59-7.50 (m, 2.2H), 7.26 (br d, J=7.3 Hz, 1.2H), 7.23-7.15 (m, 0.8H), 5.73 (s, 0.66H), 5.37 (s, 0.34H), 3.51-3.36 (m, 0.5H), 3.05 (br dd, J=6.4, 5.3 Hz, 0.5H), 2.98-2.90 (m, 0.8H), 2.67-2.62 (m, 0.6H), 2.55-2.52 (m, 3H), 1.53-1.43 (m, 2H), 1.35-1.25 (m, 2H), 1.14 (s, 6H), 1.12 (s, 3H), 1.03 (s, 2H), 0.99 (s, 1H), 0.85 (s, 1H), 0.81 (s, 4H).

### Example 155

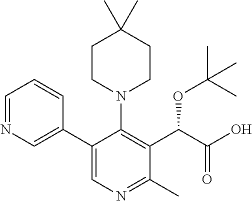 [SMILES: Cc1ncc(-c2cccnc2)c(N2CCC(C)(C)CC2)c1[C@H](OC(C)(C)C)C(=O)O]

### (2S)-2-(tert-Butoxy)-2-[4-(4, 4-dimethylpiperidin-1-yl)-6-methyl-[3, 3′-bipyridine]-5-yl]acetic acid

General method C was followed on a 55 μmol reaction scale using pyridin-3-ylboronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/to afford (2S)-2-(tert-butoxy)-2-[4-(4,4-dimethylpiperidin-1-yl)-6-methyl-[3,3′-bipyridine]-5-yl]acetic acid (14.4 mg, 64%). LCMS (M+H)=412.12. ¹H NMR (500 MHz, DMSO-d6) δ 8.66-8.60 (m, 1H), 8.54 (d, J=1.5 Hz, 1H), 8.08 (s, 1H), 7.80-7.72 (m, 1H), 7.50 (dd, J=7.7, 4.8 Hz, 1H), 5.83 (s, 1H), 2.52 (s, 3H), 1.35-1.24 (m, 4H), 1.14 (s, 9H), 0.79 (br s, 6H).

### Example 156

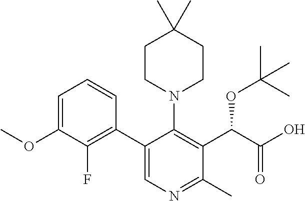 [SMILES: COc1cccc(-c2cnc(C)c([C@H](OC(C)(C)C)C(=O)O)c2N2CCC(C)(C)CC2)c1F]

### (2S)-2-(tert-Butoxy)-2-[4-(4, 4-dimethylpiperidin-1-yl)-5-(2-fluoro-3-methoxyphenyl)-2-methylpyridin-3-yl]acetic acid

General method C was followed on a 55 μmol reaction scale using (2-fluoro-3-methoxyphenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-5-(2-fluoro-3-methoxyphenyl)-2-methylpyridin-3-yl]acetic acid (10.3 mg, 41%).

LCMS (M+H)=459.22. Ratio of atropisomers is 57:43 as determined by ¹H NMR (500 MHz, methanol-d4) δ 5.90 (br s, 1H), 5.83 (br s, 0.75H).

¹H NMR (500 MHz, methanol-d4) δ 8.07-7.95 (m, 1H), 7.25-7.13 (m, 2H), 6.94-6.78 (m, 1H), 5.90 (br s, 0.6H), 5.83 (br s, 0.4H), 5.83 (br d, J=1.5 Hz, 1H), 3.93 (s, 3H), 3.12-2.92 (m, 2H), 2.90-2.68 (m, 2H), 2.62 (s, 3H), 1.39 (br s, 4H), 1.20 (s, 9H), 0.84 (br s, 6H).

### Example 157

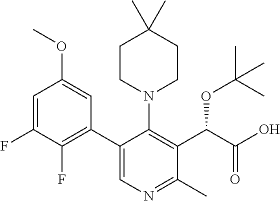 [SMILES: COc1cc(F)c(F)c(-c2cnc(C)c([C@H](OC(C)(C)C)C(=O)O)c2N2CCC(C)(C)CC2)c1]

### (2S)-2-(tert-Butoxy)-2-[5-(2, 3-difluoro-5-methoxyphenyl)-4-(4, 4-dimethylpiperidin-1-yl)-2-methylpyridin-3-yl]acetic acid

General method C was followed on a 55 μmol reaction scale using (2,3-difluoro-5-methoxyphenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[5-(2,3-difluoro-5-methoxyphenyl)-4-(4,4-dimethylpiperidin-1-yl)-2-methylpyridin-3-yl]acetic acid (11.2 mg, 43%). LCMS (M+H)=477.24. ¹H NMR (500 MHz, methanol-d4) δ 8.05 (br s, 1H), 7.01-6.93 (m, 1H), 6.74-6.59 (m, 1H), 5.96-5.81 (m, 1H), 3.82 (s, 3H), 3.12-2.92 (m, 2H), 2.90-2.68 (m, 2H), 2.62 (s, 3H), 1.41 (br s, 4H), 1.20 (s, 9H), 0.87 (br s, 6H)

### Example 158

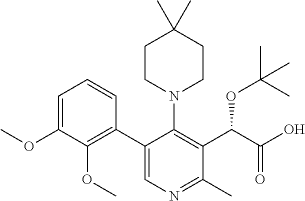 [SMILES: COc1cccc(-c2cnc(C)c([C@H](OC(C)(C)C)C(=O)O)c2N2CCC(C)(C)CC2)c1OC]

### (2S)-2-(tert-Butoxy)-2-[5-(2, 3-dimethoxyphenyl)-4-(4, 4-dimethylpiperidin-1-yl)-2-methylpyridin-3-yl]acetic acid

General method C was followed on a 55 μmol reaction scale using (2,3-dimethoxyphenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[5-(2,3-dimethoxyphenyl)-4-(4,4-dimethylpiperidin-1-yl)-2-methylpyridin-3-yl]acetic acid (14.5 mg, 56%). LCMS (M+H)=471.15 and 471.15. Ratio of atropisomers is 65:35 as determined by ¹H NMR (500 MHz, methanol-d4) δ 5.82 (s, 1H), 5.73 (s, 0.56H).

¹H NMR (500 MHz, methanol-d4) δ 8.01 (s, 0.65H), 7.97 (s, 0.35H), 7.20-7.10 (m, 2H), 6.84 (dd, J=7.0, 2.4 Hz, 0.35H), 6.80 (dd, J=7.0, 1.8 Hz, 0.65H), 5.82 (s, 0.65H), 5.73 (s, 0.35H), 3.94 (s, 3H), 3.77 (s, 1.8H), 3.53 (s, 1H), 3.11 (br s, 2H), 2.94 (br s, 0.70H), 2.84-2.76 (m, 1.4H), 2.66 (s, 3H), 2.65 (s, 3H), 2.00 (s, 1.5H), 1.40 (br s, 4H), 1.22-1.19 (m, 9H), 0.88 (s, 2H), 0.85 (s, 4H).

### Example 159

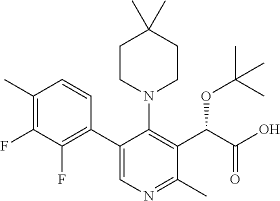 [SMILES: Cc1ccc(-c2cnc(C)c([C@H](OC(C)(C)C)C(=O)O)c2N2CCC(C)(C)CC2)c(F)c1F]

### (2S)-2-(tert-Butoxy)-2-[5-(2, 3-difluoro-4-methylphenyl)-4-(4, 4-dimethylpiperidin-1-yl)-2-methylpyridin-3-yl]acetic acid

General method C was followed on a 55 μmol reaction scale using (2,3-difluoro-4-methylphenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[5-(2,3-difluoro-4-methylphenyl)-4-(4,4-dimethylpiperidin-1-yl)-2-methylpyridin-3-yl]acetic acid (17.6 mg, 70%). LCMS (M+H)=461.22. ¹H NMR (500 MHz, methanol-d4) δ 8.08-7.97 (m, 1H), 7.15 (br t, J=7.6 Hz, 1H), 6.98 (br s, 1H), 5.89 (br s, 1H), 3.00 (br s, 2H), 2.75 (br s, 2H), 2.62 (s, 3H), 2.38 (d, J=1.8 Hz, 3H), 1.46-1.34 (m, 4H), 1.20 (s, 9H), 0.86 (br s, 6H).

### Example 160

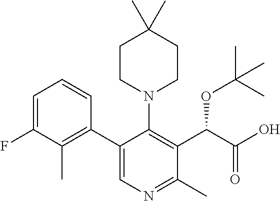 [SMILES: Cc1ncc(-c2cccc(F)c2C)c(N2CCC(C)(C)CC2)c1[C@H](OC(C)(C)C)C(=O)O]

### (2S)-2-(tert-Butoxy)-2-[4-(4, 4-dimethylpiperidin-1-yl)-5-(3-fluoro-2-methylphenyl)-2-methylpyridin-3-yl]acetic acid

General method C was followed on a 55 μmol reaction scale using (3-fluoro-2-methylphenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-5-(3-fluoro-2-methylphenyl)-2-methylpyridin-3-yl]acetic acid (8.6 mg, 35%). LCMS (M+H)=443.25 and 443.25. Ratio of atropisomers is 75:25 as determined by ¹H NMR (500 MHz, methanol-d4) δ 5.92 (s, 1H), 5.81 (s, 0.33H). ¹H NMR (500 MHz, methanol-d4) δ 7.98 (s, 0.75H), 7.91 (s, 0.25H), 7.36-7.29 (m, 1H), 7.17 (t, J=8.9 Hz, 1H), 7.08 (d, J=7.3 Hz, 0.25H), 7.01 (d, J=7.6 Hz, 0.75H), 5.92 (s, 0.75H), 5.81 (s, 0.25H), 3.10-2.84 (m, 2.5H), 2.76-2.68 (m, 1.5H), 2.68-2.64 (m, 3H), 2.11 (d, J=2.1 Hz, 2.25H), 2.01 (d, J=2.4 Hz, 0.75H), 1.43-1.34 (m, 4H), 1.23 (s, 9H), 0.86-0.83 (m, 6H).

### Example 161

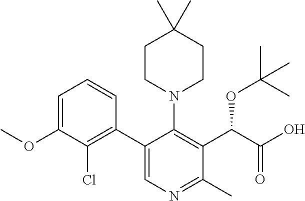 [SMILES: COc1cccc(-c2cnc(C)c([C@H](OC(C)(C)C)C(=O)O)c2N2CCC(C)(C)CC2)c1Cl]

### (2S)-2-(tert-Butoxy)-2-[5-(2-chloro-3-methoxyphenyl)-4-(4, 4-dimethylpiperidin-1-yl)-2-methylpyridin-3-yl]acetic acid

The Suzuki cross-coupling procedure of general method C was followed on a 55 μmol reaction scale using (2-chloro-3-methoxyphenyl)boronic acid and (S)-isopropyl 2-(5-bromo-4-(4,4-dimethylpiperidin-1-yl)-2-methylpyridin-3-yl)-2-(tert-butoxy)acetate as starting material. The crude ester intermediate was purified as follows: The crude sample was dissolved in a min of acetone, then concentrated onto Celite in vacuo. The resulting powder was subjected to SiO₂ purification (24 g column, hexanes:EtOAc 100:0-60:40). The product fractions were selected based on mass purity, then were pooled and concentrated in vacuo. The resulting residue was transferred to a 7 mL vial using acetone, then was concentrated under a N₂ stream. The saponification procedure of general method C was followed. The crude material was purified via preparative LC/MS to afford (2S)-2-(tert-butoxy)-2-[5-(2-chloro-3-methoxyphenyl)-4-(4,4-dimethylpiperidin-1-yl)-2-methylpyridin-3-yl]acetic acid (13.1 mg, 50%). LCMS (M+H)=475.23 and 475.23. Ratio of atropisomers is 75:25 as determined by ¹H NMR (500 MHz, methanol-d4) δ 5.88 (s, 1H), 5.77-5.72 (m, 0.32H).

¹H NMR (500 MHz, methanol-d4) δ 7.97 (s, 0.75H), 7.87 (s, 0.25H), 7.43-7.33 (m, 1.25H), 7.18 (d, J=8.5 Hz, 1H), 6.97 (d, J=7.3 Hz, 0.25H), 6.91 (d, J=7.6 Hz, 1H), 5.88 (s, 0.75H), 5.74 (s, 0.25H), 3.95 (s, 3H), 3.15-3.02 (m, 0.5H), 3.02-2.87 (m, 1.75H), 2.83-2.72 (m, 1.75H), 2.66-2.58 (m, 3H), 1.43-1.31 (m, 4H), 1.23-1.16 (m, 9H), 0.85-0.79 (m, 6H).

### Example 162

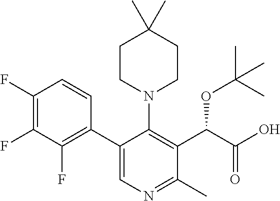 [SMILES: Cc1ncc(-c2ccc(F)c(F)c2F)c(N2CCC(C)(C)CC2)c1[C@H](OC(C)(C)C)C(=O)O]

### (2S)-2-(tert-Butoxy)-2-[4-(4, 4-dimethylpiperidin-1-yl)-2-methyl-5-(2,3, 4-trifluorophenyl)pyridin-3-yl]acetic acid

General method C was followed on a 55 μmol reaction scale using (2,3,4-trifluorophenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-2-methyl-5-(2,3,4-trifluorophenyl)pyridin-3-yl]acetic acid (11.1 mg, 43%). LCMS (M+H)=465.21. ¹H NMR (500 MHz, methanol-d4) δ 8.00 (br s, 1H), 7.27-7.19 (m, 1H), 7.12 (br s, 1H), 5.83 (br s, 1H), 3.04 (br s, 2H), 2.71 (br s, 2H), 2.62 (s, 3H), 1.46-1.35 (m, 4H), 1.19 (s, 9H), 0.86 (br s, 6H).

### Example 163

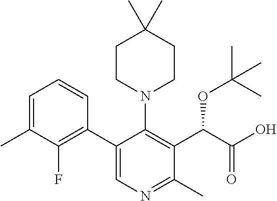 [SMILES: Cc1cccc(-c2cnc(C)c([C@H](OC(C)(C)C)C(=O)O)c2N2CCC(C)(C)CC2)c1F]

### (2S)-2-(tert-Butoxy)-2-[4-(4, 4-dimethylpiperidin-1-yl)-5-(2-fluoro-3-methylphenyl)-2-methylpyridin-3-yl]acetic acid

General method C was followed on a 55 μmol reaction scale using (2-fluoro-3-methylphenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-5-(2-fluoro-3-methylphenyl)-2-methylpyridin-3-yl]acetic acid (9.2 mg, 38%). LCMS (M+H)=443.27. Ratio of atropisomers is 60:40 as determined by ¹H NMR (500 MHz, methanol-d4) δ 5.94-5.88 (m, 1H), 5.83 (br s, 0.7H).

¹H NMR (500 MHz, methanol-d4) δ 8.09-7.94 (m, 1H), 7.39-7.33 (m, 1H), 7.23-7.17 (m, 1H), 7.18-7.07 (m, 1H), 5.91 (br s, 0.6H), 5.83 (br s, 0.4H), 3.03 (br s, 1.8H), 2.86 (br s, 0.8H), 2.75 (br s, 1H), 2.65 (s, 3H), 2.35 (s, 3H), 1.40 (br s, 4H), 1.22 (br s, 9H), 0.90-0.82 (m, 6H)

### Example 164

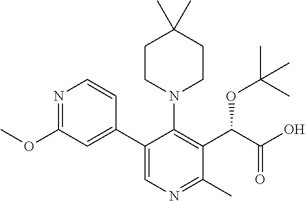 [SMILES: COc1cc(-c2cnc(C)c([C@H](OC(C)(C)C)C(=O)O)c2N2CCC(C)(C)CC2)ccn1]

### (2S)-2-(tert-Butoxy)-2-[4-(4, 4-dimethylpiperidin-1-yl)-2′-methoxy-6-methyl-[3, 4′-bipyridine]-5-yl]acetic acid

General method C was followed on a 55 μmol reaction scale using (2-methoxypyridin-4-yl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-2′-methoxy-6-methyl-[3,4′-bipyridine]-5-yl]acetic acid (10.4 mg, 49%). LCMS (M+H)=442.16. ¹H NMR (500 MHz, methanol-d4) δ 8.22 (d, J=5.2 Hz, 1H), 8.04 (s, 1H), 6.95 (dd, J=5.2, 1.2 Hz, 1H), 6.79 (s, 1H), 5.87 (s, 1H), 3.98 (s, 3H), 3.09 (br s, 2H), 2.74 (br s, 2H), 2.62 (s, 3H), 1.50-1.37 (m, 4H), 1.19 (s, 9H), 0.88 (s, 6H).

### Example 165

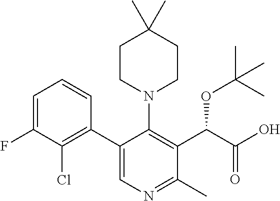 [SMILES: Cc1ncc(-c2cccc(F)c2Cl)c(N2CCC(C)(C)CC2)c1[C@H](OC(C)(C)C)C(=O)O]

### (2S)-2-(tert-Butoxy)-2-[5-(2-chloro-3-fluorophenyl)-4-(4, 4-dimethylpiperidin-1-yl)-2-methylpyridin-3-yl]acetic acid

The Suzuki cross-coupling procedure of general method C was followed on a 55 mol reaction scale using (2-chloro-3-fluorophenyl)boronic acid and (S)-isopropyl 2-(5-bromo-4-(4,4-dimethylpiperidin-1-yl)-2-methylpyridin-3-yl)-2-(tert-butoxy)acetate as starting material. The crude ester intermediate was purified as follows: The crude sample was dissolved in a min of acetone, then concentrated onto Celite in vacuo. The resulting powder was subjected to SiO2 purification (24 g column, hexanes:EtOAc 100:0-60:40). The product fractions were selected based on mass purity, then were pooled and concentrated in vacuo. The resulting residue was transferred to a 7 mL vial using acetone, then was concentrated under a N₂ stream. The saponification procedure of general method C was followed. The crude material was purified via preparative LC/MS to afford (2S)-2-(tert-butoxy)-2-[5-(2-chloro-3-fluorophenyl)-4-(4,4-dimethylpiperidin-1-yl)-2-methylpyridin-3-yl]acetic acid (3.9 mg, 15%). LCMS (M+H)=463.17 and 463.17. Ratio of atropisomers is 74:26 as determined by ¹H NMR (500 MHz, methanol-d4) δ 5.94 (s, 1.0H), 5.82 (s, 0.35H).

¹H NMR (500 MHz, methanol-d4) δ 8.03 (s, 1H), 7.93 (s, 0.3H), 7.51-7.42 (m, 1.3H), 7.41-7.34 (m, 1.3H), 7.26 (d, J=8.2 Hz, 0.3H), 7.19 (d, J=7.6 Hz, 1H), 5.94 (s, 1H), 5.82 (s, 0.3H), 3.08-2.87 (m, 2.6H), 2.83-2.74 (m, 2.6H), 2.65 (s, 1H), 2.64 (s, 3H), 1.43-1.36 (m, 5.2H), 1.23 (s, 12H), 0.87 (s, 2H), 0.85 (s, 6H).

### Example 166

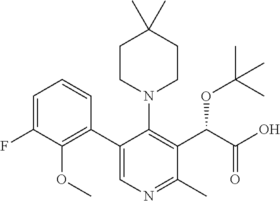 [SMILES: COc1c(F)cccc1-c1cnc(C)c([C@H](OC(C)(C)C)C(=O)O)c1N1CCC(C)(C)CC1]

### (2S)-2-(tert-Butoxy)-2-[4-(4, 4-dimethylpiperidin-1-yl)-5-(3-fluoro-2-methoxyphenyl)-2-methylpyridin-3-yl]acetic acid

General method C was followed on a 55 μmol reaction scale using (3-fluoro-2-methoxyphenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-5-(3-fluoro-2-methoxyphenyl)-2-methylpyridin-3-yl]acetic acid (10.4 mg, 41%). LCMS (M+H)=459.23. Ratio of atropisomers is 61:39 as determined by ¹H NMR (500 MHz, methanol-d4) δ 5.88 (s, 1H), 5.78 (s, 1H).

¹H NMR (500 MHz, methanol-d4) δ 8.04 (s, 1H), 7.99 (s, 0.6H), 7.30-7.23 (m, 1.6H), 7.17 (td, J=8.0, 5.0 Hz, 1.6H), 7.07 (br d, J=7.9 Hz, 0.6H), 7.01 (br d, J=7.0 Hz, 1H), 5.88 (s, 1H), 5.78 (s, 0.6H), 3.89 (s, 3H), 3.68 (s, 1.8H), 3.13-2.97 (m, 2.8H), 2.97-2.87 (m, 1.3H), 2.83-2.74 (m, 2H), 2.65 (s, 4.8H), 1.40 (br s, 6.4H), 1.22 (s, 9H), 1.21 (s, 5H), 0.89 (s, 3.6H), 0.85 (s, 6H).

### Example 167

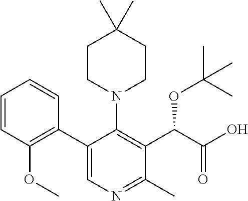 [SMILES: COc1ccccc1-c1cnc(C)c([C@H](OC(C)(C)C)C(=O)O)c1N1CCC(C)(C)CC1]

### (2S)-2-(tert-Butoxy)-2-[4-(4, 4-dimethylpiperidin-1-yl)-5-(2-methoxyphenyl)-2-methylpyridin-3-yl]acetic acid

General method C was followed on a 55 μmol reaction scale using (2-methoxyphenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-5-(2-methoxyphenyl)-2-methylpyridin-3-yl]acetic acid (11.4 mg, 47%). LCMS (M+H)=441.25. Ratio of atropisomers is 59:41 ratio as determined by ¹H NMR (500 MHz, DMSO-d6) δ 5.90 (s, 1H), 5.82 (s, 0.7H).

¹H NMR (500 MHz, DMSO-d6) δ 7.93 (s, 1H), 7.87 (s, 0.6H), 7.46-7.38 (m, 2H), 7.18-7.12 (m, 2H), 7.11-7.00 (m, 3.3H), 5.90 (s, 1H), 5.82 (s, 0.7H), 3.73 (s, 3H), 3.71 (s, 2.1H), 2.49 (s, 5H), 1.28 (br s, 4H), 1.17-1.14 (m, 9H), 1.13 (s, 6.5H), 0.82-0.68 (m, 10H).

### Example 168

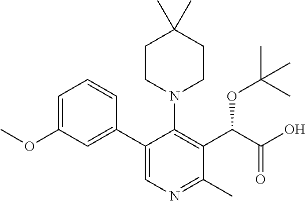 [SMILES: COc1cccc(-c2cnc(C)c([C@H](OC(C)(C)C)C(=O)O)c2N2CCC(C)(C)CC2)c1]

### (2S)-2-(tert-Butoxy)-2-[4-(4, 4-dimethylpiperidin-1-yl)-5-(3-methoxyphenyl)-2-methylpyridin-3-yl]acetic acid

General method C was followed on a 55 μmol reaction scale using (3-methoxyphenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-5-(3-methoxyphenyl)-2-methylpyridin-3-yl]acetic acid (12.9 mg, 53%). LCMS (M+H)=441.24. ¹H NMR (500 MHz, methanol-d4) δ 8.85 (br d, J=4.9 Hz, 1H), 8.23-8.13 (m, 1H), 7.84-7.75 (m, 1H), 7.72-7.65 (m, 2H), 6.66 (br s, 1H), 4.62 (s, 3H), 3.36 (br s, 3H), 2.16-2.05 (m, 4H), 1.95 (br s, 9H), 1.67-1.58 (m, 6H).

### Example 169

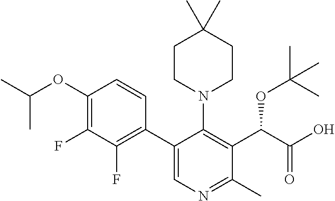 [SMILES: Cc1ncc(-c2ccc(OC(C)C)c(F)c2F)c(N2CCC(C)(C)CC2)c1[C@H](OC(C)(C)C)C(=O)O]

### (2S)-2-(tert-Butoxy)-2-{5-[2, 3-difluoro-4-(propan-2-yloxy)phenyl]-4-(4, 4-dimethylpiperidin-1-yl)-2-methylpyridin-3-yl}acetic acid

General method C was followed on a 55 μmol reaction scale using (2,3-difluoro-4-isopropoxyphenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-{5-[2,3-difluoro-4-(propan-2-yloxy)phenyl]-4-(4,4-dimethylpiperidin-1-yl)-2-methylpyridin-3-yl}acetic acid (14.5 mg, 52%). LCMS (M+H)=505.26. ¹H NMR (500 MHz, methanol-d4) δ 8.06 (br s, 1H), 7.10-6.96 (m, 2H), 5.93 (br s, 1H), 4.72 (dt, J=12.2, 6.1 Hz, 2H), 3.02 (br s, 2H), 2.78 (br s, 2H), 2.64 (s, 3H), 1.40 (dd, J=6.1, 3.1 Hz, 4H), 1.45-1.37 (m, 4H), 1.22 (s, 9H), 0.88 (br s, 6H).

### Example 170

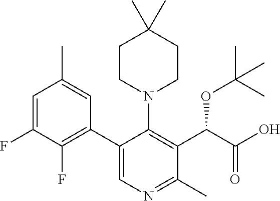 [SMILES: Cc1cc(F)c(F)c(-c2cnc(C)c([C@H](OC(C)(C)C)C(=O)O)c2N2CCC(C)(C)CC2)c1]

### (2S)-2-(tert-Butoxy)-2-[5-(2, 3-difluoro-5-methylphenyl)-4-(4, 4-dimethylpiperidin-1-yl)-2-methylpyridin-3-yl]acetic acid

General method C was followed on a 55 μmol reaction scale using (2,3-difluoro-5-methylphenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[5-(2,3-difluoro-5-methylphenyl)-4-(4,4-dimethylpiperidin-1-yl)-2-methylpyridin-3-yl]acetic acid (10.4 mg, 41%). LCMS (M+H)=461.22. ¹H NMR (500 MHz, DMSO-d6) δ 8.07 (s, 1H), 7.37-7.30 (m, 1H), 6.99 (s, 1H), 5.80 (br s, 1H), 2.55 (s, 3H), 2.36 (s, 3H), 1.31 (br s, 4H), 1.14 (s, 9H), 0.82 (br s, 6H).

### Example 171

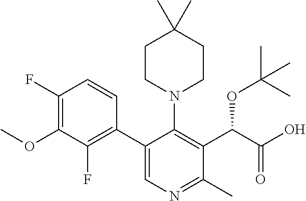 [SMILES: COc1c(F)ccc(-c2cnc(C)c([C@H](OC(C)(C)C)C(=O)O)c2N2CCC(C)(C)CC2)c1F]

### (2S)-2-(tert-Butoxy)-2-[5-(2, 4-difluoro-3-methoxyphenyl)-4-(4, 4-dimethylpiperidin-1-yl)-2-methylpyridin-3-yl]acetic acid

General method C was followed on a 55 μmol reaction scale using (2,4-difluoro-3-methoxyphenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[5-(2,4-difluoro-3-methoxyphenyl)-4-(4,4-dimethylpiperidin-1-yl)-2-methylpyridin-3-yl]acetic acid (12.6 mg, 48%). LCMS (M+H)=477.21. Ratio of atropisomers is 1:1 as determined by ¹H NMR (500 MHz, methanol-d4) δ 5.95-5.89 (m, 1H), 5.89-5.82 (m, 1H).

¹H NMR (500 MHz, methanol-d4) δ 8.13-7.91 (m, 1H), 7.14-7.07 (m, 1H), 7.07-6.91 (m, 1H), 5.98-5.79 (m, 1H), 4.01 (s, 3H), 3.11-2.88 (m, 2H), 2.86-2.67 (m, 2H), 2.62 (s, 3H), 1.40 (br dd, J=6.0, 4.1 Hz, 4H), 1.20 (s, 9H), 0.86 (br s, 6H).

### Example 172 Example 173

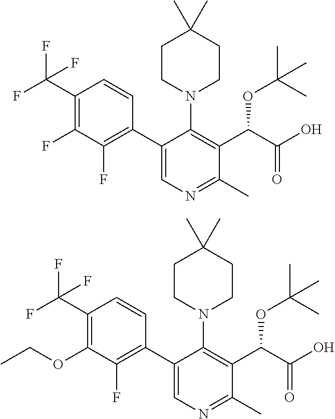

### (2S)-2-(tert-Butoxy)-2-{5-[2, 3-difluoro-4-(trifluoromethyl)phenyl]-4-(4, 4-dimethylpiperidin-1-yl)-2-methylpyridin-3-yl}acetic acid and (2S)-2-(tert-butoxy)-2-[4-(4, 4-dimethylpiperidin-1-yl)-5-[3-ethoxy-2-fluoro-4-(trifluoromethyl)phenyl]-2-methylpyridin-3-yl]acetic acid

General method C was followed on a 55 μmol reaction scale using (2,3-difluoro-4-(trifluoromethyl)phenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/to afford (2S)-2-(tert-butoxy)-2-{5-[2,3-difluoro-4-(trifluoromethyl)phenyl]-4-(4,4-dimethylpiperidin-1-yl)-2-methylpyridin-3-yl}acetic acid (4.3 mg, 15%) and (2S)-2-(tert-butoxy)-2-[4-(4,4-dimethylpiperidin-1-yl)-5-[3-ethoxy-2-fluoro-4-(trifluoromethyl)phenyl]-2-methylpyridin-3-yl]acetic acid (6 mg, 20%).

### Analysis of Example 172:

LCMS (M+H)=515.2. ¹H NMR (500 MHz, methanol-d4) δ 8.15-8.00 (m, 1H), 7.62 (br t, J=7.5 Hz, 1H), 7.44-7.24 (m, 1H), 5.96-5.81 (m, 1H), 2.96 (br s, 2H), 2.77 (br s, 2H), 2.63 (s, 3H), 1.45-1.35 (m, 4H), 1.21 (s, 9H), 0.86 (br s, 6H).

### Analysis of Example 173:

LCMS (M+H)=541.26. Ratio of atropisomers is 1:1 as determined by ¹H NMR (500 MHz, methanol-d4) δ 5.97-5.90 (m, 1H), 5.89-5.82 (m, 1H).

¹H NMR (500 MHz, Methanol-d4) δ 8.11-7.97 (m, 1H), 7.52 (d, J=8.9 Hz, 1H), 7.29-7.06 (m, 1H), 5.98-5.80 (m, 1H), 4.32-4.19 (m, 2H), 3.08-2.88 (m, 2H), 2.87-2.68 (m, 2H), 2.63 (s, 3H), 1.45-1.35 (m, 7H), 1.20 (s, 9H), 0.86 (br d, J=15.6 Hz, 6H).

### Example 174

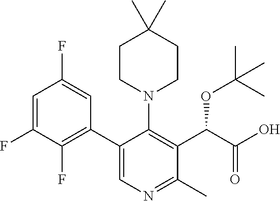 [SMILES: Cc1ncc(-c2cc(F)cc(F)c2F)c(N2CCC(C)(C)CC2)c1[C@H](OC(C)(C)C)C(=O)O]

### (2S)-2-(tert-Butoxy)-2-[4-(4, 4-dimethylpiperidin-1-yl)-2-methyl-5-(2, 3,5-trifluorophenyl)pyridin-3-yl]acetic acid

General method C was followed on a 55 μmol reaction scale using (2,3,5-trifluorophenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-2-methyl-5-(2,3,5-trifluorophenyl)pyridin-3-yl]acetic acid (8.5 mg, 33%). LCMS (M+H)=465.18. ¹H NMR (500 MHz, DMSO-d6) δ 8.16-8.01 (m, 1H), 7.65-7.53 (m, 1H), 7.18 (br s, 1H), 5.78 (br s, 1H), 2.52 (s, 3H), 1.31 (br s, 4H), 1.14 (s, 9H), 0.82 (br s, 6H).

### Example 175

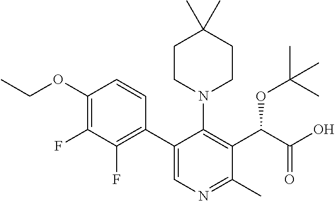 [SMILES: CCOc1ccc(-c2cnc(C)c([C@H](OC(C)(C)C)C(=O)O)c2N2CCC(C)(C)CC2)c(F)c1F]

### (2S)-2-(tert-Butoxy)-2-[4-(4, 4-dimethylpiperidin-1-yl)-5-(4-ethoxy-2, 3-difluorophenyl)-2-methylpyridin-3-yl]acetic acid

General method C was followed on a 55 μmol reaction scale using (4-ethoxy-2,3-difluorophenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-5-(4-ethoxy-2,3-difluorophenyl)-2-methylpyridin-3-yl]acetic acid (11.4 mg, 42%). LCMS (M+H)=491.24. ¹H NMR (500 MHz, DMSO-d6) δ 8.05 (br s, 1H), 7.18-7.03 (m, 2H), 5.88-5.76 (m, 1H), 4.23 (q, J=7.0 Hz, 2H), 2.55 (s, 3H), 1.39 (t, J=7.0 Hz, 3H), 1.31 (br s, 4H), 1.15 (s, 9H), 0.90-0.73 (m, 6H).

### Example 176

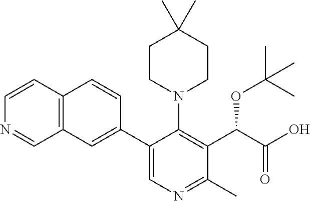 [SMILES: Cc1ncc(-c2ccc3ccncc3c2)c(N2CCC(C)(C)CC2)c1[C@H](OC(C)(C)C)C(=O)O]

### (2S)-2-(tert-Butoxy)-2-[4-(4, 4-dimethylpiperidin-1-yl)-5-(isoquinolin-7-yl)-2-methylpyridin-3-yl]acetic acid

General method C was followed on a 55 μmol reaction scale using isoquinolin-7-ylboronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-5-(isoquinolin-7-yl)-2-methylpyridin-3-yl]acetic acid (5.9 mg, 23%). LCMS (M+H)=462.2. Ratio of atropisomers is 55:45 as determined by ¹H NMR (500 MHz, methanol-d4) δ 6.00 (br s, 1H), 5.84-5.80 (m, 0.85H); Ratio of atropisomers is 69:31 by HPLC.

¹H NMR (500 MHz, methanol-d4) δ 8.99-8.91 (m, 1H), 8.81 (br s, 0.8H), 8.51 (br d, J=4.6 Hz, 2H), 8.18-8.10 (m, 2H), 8.07 (br t, J=7.0 Hz, 1.8H), 7.96-7.87 (m, 3.7H), 7.65 (d, J=7.0 Hz, 0.8H), 7.59 (d, J=6.7 Hz, 1H), 6.00 (br s, 1H), 5.82 (s, 0.8H), 2.70 (s, 6H), 1.24 (s, 9H), 1.31-1.21 (m, 3.2H), 1.22 (s, 7.2H), 1.18-1.12 (m, 3.2H), 0.72 (s, 6H), 0.56-0.49 (m, 4.8H).

### Example 177

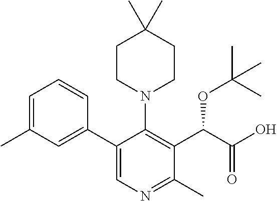 [SMILES: Cc1cccc(-c2cnc(C)c([C@H](OC(C)(C)C)C(=O)O)c2N2CCC(C)(C)CC2)c1]

### (2S)-2-(tert-Butoxy)-2-[4-(4, 4-dimethylpiperidin-1-yl)-2-methyl-5-(3-methylphenyl)pyridin-3-yl]acetic acid

General method C was followed on a 55 μmol reaction scale using m-tolylboronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-2-methyl-5-(3-methylphenyl)pyridin-3-yl]acetic acid (9.4 mg, 40%). LCMS (M+H)=425.24. ¹H NMR (500 MHz, DMSO-d6) δ 8.04 (s, 1H), 7.36 (t, J=7.5 Hz, 1H), 7.24 (br d, J=8.2 Hz, 1H), 7.14 (s, 1H), 7.11 (d, J=8.5 Hz, 1H), 5.87-5.83 (m, 1H), 2.55 (s, 3H), 2.38 (s, 3H), 1.35-1.26 (m, 4H), 1.23 (s, 3H), 1.14 (s, 9H), 0.84-0.74 (m, 6H).

### Example 178

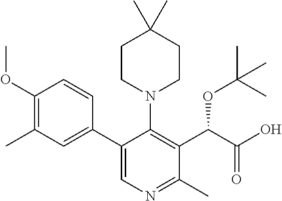 [SMILES: COc1ccc(-c2cnc(C)c([C@H](OC(C)(C)C)C(=O)O)c2N2CCC(C)(C)CC2)cc1C]

### (2S)-2-(tert-Butoxy)-2-[4-(4, 4-dimethylpiperidin-1-yl)-5-(4-methoxy-3-methylphenyl)-2-methylpyridin-3-yl]acetic acid

General method C was followed on a 55 μmol reaction scale using (4-methoxy-3-methylphenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-5-(4-methoxy-3-methylphenyl)-2-methylpyridin-3-yl]acetic acid (19.8 mg, 79%). LCMS (M+H)=455.24. ¹H NMR (500 MHz, methanol-d4) δ 8.11-7.91 (m, 1H), 7.19-7.08 (m, 2H), 7.03 (br d, J=7.9 Hz, 1H), 5.97-5.52 (br s, 1H), 3.90 (s, 3H), 3.27-3.01 (m, 2H), 2.90-2.59 (m, 5H), 2.27 (s, 3H), 1.50-1.36 (m, 4H), 1.36-1.13 (m, 9H), 0.89 (s, 6H).

### Example 179

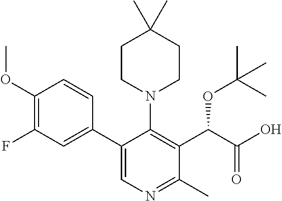 [SMILES: COc1ccc(-c2cnc(C)c([C@H](OC(C)(C)C)C(=O)O)c2N2CCC(C)(C)CC2)cc1F]

### (2S)-2-(tert-Butoxy)-2-[4-(4, 4-dimethylpiperidin-1-yl)-5-(3-fluoro-4-methoxyphenyl)-2-methylpyridin-3-yl]acetic acid

General method C was followed on a 55 μmol reaction scale using (3-fluoro-4-methoxyphenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-5-(3-fluoro-4-methoxyphenyl)-2-methylpyridin-3-yl]acetic acid (13.5 mg, 54%). LCMS (M+H)=459.23. ¹H NMR (500 MHz, methanol-d4) δ 8.06 (s, 1H), 7.27-7.19 (m, 1H), 7.16-7.06 (m, 2H), 5.86 (s, 1H), 3.96 (s, 3H), 3.18-3.00 (m, 2H), 2.83-2.71 (m, 2H), 2.65 (s, 3H), 1.44 (br d, J=4.3 Hz, 4H), 1.22 (s, 9H), 0.90 (s, 6H).

### Example 180

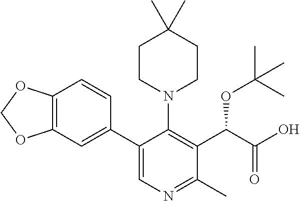 [SMILES: Cc1ncc(-c2ccc3c(c2)OCO3)c(N2CCC(C)(C)CC2)c1[C@H](OC(C)(C)C)C(=O)O]

### (2S)-2-[5-(2H-1, 3-Benzodioxol-5-yl)-4-(4, 4-dimethylpiperidin-1-yl)-2-methylpyridin-3-yl]-2-(tert-butoxy)acetic acid

General method C was followed on a 55 μmol reaction scale using benzo[d][1,3]dioxol-5-ylboronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-[5-(2H-1,3-benzodioxol-5-yl)-4-(4,4-dimethylpiperidin-1-yl)-2-methylpyridin-3-yl]-2-(tert-butoxy)acetic acid (12.9 mg, 52%). LCMS (M+H)=455.34. ¹H NMR (500 MHz, methanol-d4) δ 8.04 (s, 1H), 6.95 (d, J=7.9 Hz, 1H), 6.84 (d, J=1.5 Hz, 1H), 6.80 (dd, J=8.1, 1.7 Hz, 1H), 6.04 (s, 2H), 5.83 (s, 1H), 3.23-3.08 (m, 2H), 2.83-2.72 (m, 2H), 2.65 (s, 3H), 1.51-1.38 (m, 4H), 1.21 (s, 9H), 0.91 (s, 6H).

### Example 181

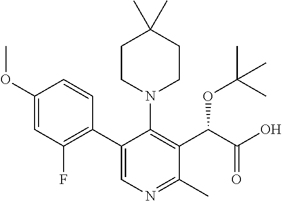 [SMILES: COc1ccc(-c2cnc(C)c([C@H](OC(C)(C)C)C(=O)O)c2N2CCC(C)(C)CC2)c(F)c1]

### (2S)-2-(tert-Butoxy)-2-[4-(4, 4-dimethylpiperidin-1-yl)-5-(2-fluoro-4-methoxyphenyl)-2-methylpyridin-3-yl]acetic acid

General method C was followed on a 55 μmol reaction scale using (2-fluoro-4-methoxyphenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-5-(2-fluoro-4-methoxyphenyl)-2-methylpyridin-3-yl]acetic acid (8.3 mg, 33%). LCMS (M+H)=459.21. ¹H NMR (500 MHz, methanol-d4) δ 8.08-7.94 (m, 1H), 7.30-7.13 (m, 1H), 6.90 (dd, J=8.5, 2.1 Hz, 1H), 6.88-6.82 (m, 1H), 5.97-5.80 (m, 1H), 3.89 (s, 3H), 3.14-2.96 (m, 2H), 2.89-2.71 (m, 2H), 2.64 (s, 3H), 1.42 (br s, 4H), 1.22 (s, 9H), 0.88 (br s, 6H).

### Example 182

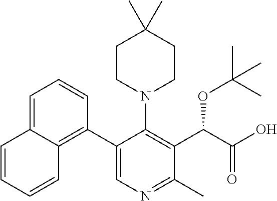 [SMILES: Cc1ncc(-c2cccc3ccccc23)c(N2CCC(C)(C)CC2)c1[C@H](OC(C)(C)C)C(=O)O]

### (2S)-2-(tert-Butoxy)-2-[4-(4, 4-dimethylpiperidin-1-yl)-2-methyl-5-(naphthalen-1-yl)pyridin-3-yl]acetic acid

General method C was followed on a 55 μmol reaction scale using naphthalen-1-ylboronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-2-methyl-5-(naphthalen-1-yl)pyridin-3-yl]acetic acid (13.5 mg, 48%). LCMS (M+H)=461.23, 461.23. Ratio of atropisomers is 53:47 as determined by ¹H NMR (500 MHz, methanol-d4) δ 8.19 (s, 0.9H), 8.16 (s, 1H).

¹H NMR (500 MHz, methanol-d4) δ 8.19 (s, 1H), 8.16 (s, 1H), 8.06 (br d, J=10.1 Hz, 2H), 8.02 (br d, J=8.5 Hz, 2H), 7.67-7.62 (m, 2H), 7.61-7.51 (m, 6H), 7.47-7.41 (m, 2H), 5.83 (s, 1H), 5.65 (s, 1H), 2.97-2.86 (m, 4H), 2.80 (s, 3H), 2.79-2.78 (m, 3H), 1.41-1.28 (m, 10H), 1.27 (s, 9H), 1.24 (s, 8H), 1.21-1.10 (m, 4H), 0.78 (s, 6H), 0.58 (s, 5H).

### Example 183

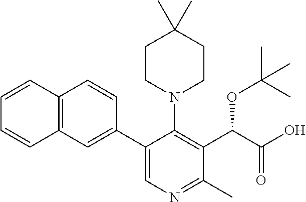 [SMILES: Cc1ncc(-c2ccc3ccccc3c2)c(N2CCC(C)(C)CC2)c1[C@H](OC(C)(C)C)C(=O)O]

### (2S)-2-(tert-Butoxy)-2-[4-(4, 4-dimethylpiperidin-1-yl)-2-methyl-5-(naphthalen-2-yl)pyridin-3-yl]acetic acid

General method C was followed on a 55 μmol reaction scale using naphthalen-2-ylboronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-2-methyl-5-(naphthalen-2-yl)pyridin-3-yl]acetic acid (14.6 mg, 58%). LCMS (M+H)=461.24. ¹H NMR (500 MHz, DMSO-d6) δ 8.15 (s, 1H), 8.05-7.96 (m, 3H), 7.88 (s, 1H), 7.61-7.53 (m, 2H), 7.48 (d, J=8.5 Hz, 1H), 5.87 (s, 1H), 2.56-2.55 (m, 3H), 1.33-1.27 (m, 2H), 1.16 (s, 9H), 0.85-0.63 (m, 5H).

### Example 184

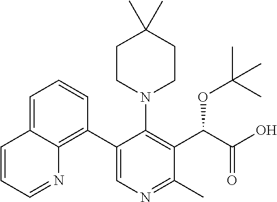 [SMILES: Cc1ncc(-c2cccc3cccnc23)c(N2CCC(C)(C)CC2)c1[C@H](OC(C)(C)C)C(=O)O]

### (2S)-2-(tert-Butoxy)-2-[4-(4, 4-dimethylpiperidin-1-yl)-2-methyl-5-(quinolin-8-yl)pyridin-3-yl]acetic acid

General method C was followed on a 55 μmol reaction scale using quinolin-8-ylboronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4-(4,4-dimethylpiperidin-1-yl)-2-methyl-5-(quinolin-8-yl)pyridin-3-yl]acetic acid (13.5 mg, 53%). LCMS (M+H)=462.2. Ratio of atropisomers is 70:30 as determined by ¹H NMR (500 MHz, methanol-d4) δ 5.84-5.80 (m, 1H), 5.72 (s, 0.45H).

¹H NMR (500 MHz, methanol-d4) δ 8.84 (d, J=2.7 Hz, 1H), 8.45-8.36 (m, 1.3H), 8.10-7.99 (m, 2.4H), 7.74-7.65 (m, 2.5H), 7.58-7.51 (m, 1.2H), 5.82 (s, 0.7H), 5.72 (s, 0.3H), 3.04-2.84 (m, 1H), 2.81-2.74 (m, 1H), 2.72 (s, 3H), 2.70-2.60 (m, 2H), 1.98 (s, 3H), 1.35-1.28 (m, 2H), 1.26 (s, 7.7H), 1.21 (s, 4.5H), 0.75 (s, 4.2H), 0.59-0.52 (m, 1.8H).

### Example 185

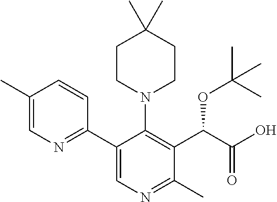 [SMILES: Cc1ccc(-c2cnc(C)c([C@H](OC(C)(C)C)C(=O)O)c2N2CCC(C)(C)CC2)nc1]

### (2S)-2-(tert-Butoxy)-2-[4′-(4, 4-dimethylpiperidin-1-yl)-5,6′-dimethyl-[2, 3′-bipyridine]-5′-yl]acetic acid

General method D was followed on a 55 μmol reaction scale using 6-methyl-2-(5-methylpyridin-2-yl)-1,3,6,2-dioxazaborocane-4,8-dione and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4′-(4,4-dimethylpiperidin-1-yl)-5,6′-dimethyl-[2,3′-bipyridine]-5′-yl]acetic acid (3.1 mg, 13%). LCMS (M+H)=426.22. ¹H NMR (500 MHz, methanol-d4) δ 8.49 (s, 1H), 8.08 (s, 1H), 7.77 (dd, J=8.1, 1.7 Hz, 1H), 7.39 (d, J=7.9 Hz, 1H), 5.79 (s, 1H), 3.00 (br s, 2H), 2.76 (br s, 2H), 2.63 (s, 3H), 2.43 (s, 3H), 1.97 (s, 3H), 1.40 (br t, J=5.2 Hz, 4H), 1.17 (s, 9H), 0.85 (s, 6H).

### Example 186

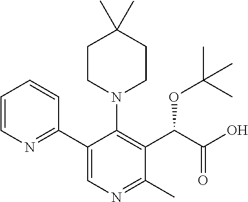 [SMILES: Cc1ncc(-c2ccccn2)c(N2CCC(C)(C)CC2)c1[C@H](OC(C)(C)C)C(=O)O]

### (2S)-2-(tert-Butoxy)-2-[4′-(4, 4-dimethylpiperidin-1-yl)-6′-methyl-[2, 3′-bipyridine]-5′-yl]acetic acid

General method D was followed on a 55 μmol reaction scale using 6-methyl-2-(pyridin-2-yl)-1,3,6,2-dioxazaborocane-4,8-dione and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-[4′-(4,4-dimethylpiperidin-1-yl)-6′-methyl-[2,3′-bipyridine]-5′-yl]acetic acid (8.7 mg, 38%). LCMS (M+H)=412.23. ¹H NMR (500 MHz, methanol-d4) δ 8.64 (d, J=4.3 Hz, 1H), 8.09 (s, 1H), 7.93 (td, J=7.8, 1.5 Hz, 1H), 7.50 (d, J=7.9 Hz, 1H), 7.45 (dd, J=7.2, 5.0 Hz, 1H), 5.80 (s, 1H), 3.00 (br s, 2H), 2.76 (br s, 2H), 2.65-2.63 (m, 3H), 1.96 (s, 3H), 1.39 (br s, 4H), 1.18 (s, 9H), 0.85 (s, 6H).

### Example 187

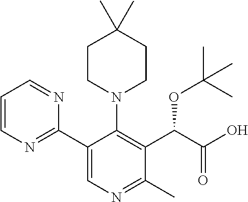 [SMILES: Cc1ncc(-c2ncccn2)c(N2CCC(C)(C)CC2)c1[C@H](OC(C)(C)C)C(=O)O]

### (2S)-2-(tert-Butoxy)-2-[4-(4, 4-dimethylpiperidin-1-yl)-2-methyl-5-(pyrimidin-2-yl)pyridin-3-yl]acetic acid

To a 14 mL test tube equipped with a stir bar was added (S)-isopropyl 2-(5-bromo-4-(4,4-dimethylpiperidin-1-yl)-2-methylpyridin-3-yl)-2-(tert-butoxy)acetate (75 mg, 0.165 mmol), cesium fluoride (25 mg, 0.165 mmol), and Pd(PPh₃)₄ (19 mg, 16.5 μmol). The test tube was sealed with a rubber septum, then placed under N₂ atmosphere. To the test tube was added a degassed (5 min sparging with N2) solution of 2-(tributylstannyl)pyrimidine in dioxane (1.5 mL). The test tube was placed in a 80° C. heating block with stirring for 24 h. The reaction mixture was cooled to r.t., then was diluted with Et₂O (5 mL) and water (5 mL). The organic phase was isolated and dried over MgSO₄, then filtered, then concentrated in a 7 mL vial under a N2 stream. To the vial was added a stir bar and EtOH (1.5 mL), then aq. sodium hydroxide (0.330 mL, 1.650 mmol). The vial was capped, then placed in a 90° C. heating block with stirring for 2 h. The mixture was cooled to r.t., then was filtered through a syringe filter to afford a solution of the crude product. The crude material was purified via preparative LC/MS to afford (2S)-2-(tert-butoxy)-2-[4-(4,4-dimethylpiperidin-1-yl)-2-methyl-5-(pyrimidin-2-yl)pyridin-3-yl]acetic acid (6 mg, 7%). LCMS (M+H)=413.07. ¹H NMR (500 MHz, methanol-d4) δ 9.00 (d, J=4.9 Hz, 2H), 8.49 (s, 1H), 7.57 (t, J=5.0 Hz, 1H), 5.83 (s, 1H), 3.03 (br d, J=18.9 Hz, 4H), 2.77 (s, 3H), 1.56-1.44 (m, 4H), 1.25 (s, 9H), 0.97 (s, 6H).

### Example 188

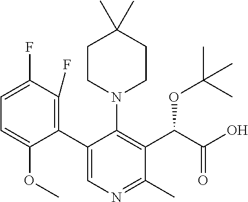 [SMILES: COc1ccc(F)c(F)c1-c1cnc(C)c([C@H](OC(C)(C)C)C(=O)O)c1N1CCC(C)(C)CC1]

### (2S)-2-(tert-Butoxy)-2-(5-(2, 3-difluoro-6-methoxyphenyl)-4-(4, 4-dimethylpiperidin-1-yl)-2-methylpyridin-3-yl)acetic acid

General method C was followed on a 55 μmol reaction scale using (2,3-difluoro-6-methoxyphenyl)boronic acid and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (2S)-2-(tert-butoxy)-2-(5-(2,3-difluoro-6-methoxyphenyl)-4-(4,4-dimethylpiperidin-1-yl)-2-methylpyridin-3-yl)acetic acid (0.7 mg, 3%). LCMS (M+H)=477.2. ¹H NMR (500 MHz, methanol-d4) δ 7.98-7.87 (m, 1H), 7.36 (q, J=9.6 Hz, 1H), 6.94-6.86 (m, 1H), 5.83 (s, 1H), 3.78 (s, 3H), 3.05 (br s, 2H), 2.84 (br s, 2H), 2.65 (s, 3H), 1.47-1.35 (m, 4H), 1.21 (s, 9H), 0.87 (s, 6H).

### Example 189

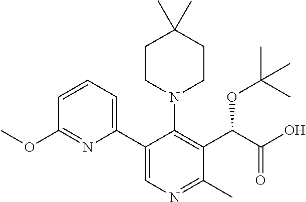 [SMILES: COc1cccc(-c2cnc(C)c([C@H](OC(C)(C)C)C(=O)O)c2N2CCC(C)(C)CC2)n1]

### (S)-2-(tert-Butoxy)-2-(4′-(4, 4-dimethylpiperidin-1-yl)-6-methoxy-6′-methyl-[2, 3′-bipyridin]-5′-yl)acetic acid

General method D was followed on a 55 μmol reaction scale using 2-(6-methoxypyridin-2-yl)-6-methyl-1,3,6,2-dioxazaborocane-4,8-dione and (S)-isopropyl 2-(5-bromo-4-(4,4-dimethylpiperidin-1-yl)-2-methylpyridin-3-yl)-2-(tert-butoxy)acetate as starting material. The crude material was purified via preparative LC/MS to afford (S)-2-(tert-butoxy)-2-(4′-(4,4-dimethylpiperidin-1-yl)-6-methoxy-6′-methyl-[2,3′-bipyridin]-5′-yl)acetic acid (4.9 mg, 17%). LCMS (M+H)=442.3. ¹H NMR (500 MHz, methanol-d4) δ 8.28 (s, 1H), 7.86-7.81 (m, 1H), 7.12 (d, J=7.0 Hz, 1H), 6.91 (d, J=8.2 Hz, 1H), 5.74 (s, 1H), 3.96 (s, 3H), 3.12-3.03 (m, 2H), 3.02-2.94 (m, 2H), 2.76 (s, 3H), 1.55-1.42 (m, 4H), 1.23 (s, 9H), 0.94 (s, 6H).

### Example 190

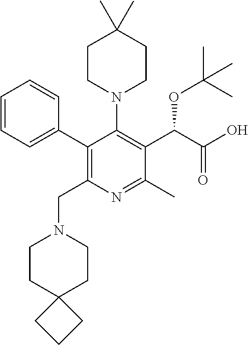 [SMILES: Cc1nc(CN2CCC3(CCC3)CC2)c(-c2ccccc2)c(N2CCC(C)(C)CC2)c1[C@H](OC(C)(C)C)C(=O)O]

### (2S)-2-[6-({7-Azaspiro[3.5]nonan-7-yl}methyl)-4-(4, 4-dimethylpiperidin-1-yl)-2-methyl-5-phenylpyridin-3-yl]-2-(tert-butoxy)acetic acid

General method B was followed on a 49 μmol reaction scale using phenylboronic acid and (S)-isopropyl 2-(6-(7-azaspiro[3.5]nonan-7-ylmethyl)-5-bromo-4-(4,4-dimethylpiperidin-1-yl)-2-methylpyridin-3-yl)-2-(tert-butoxy)acetate as starting material. The crude material was purified via preparative LC/MS to afford (2S)-2-[6-({7-azaspiro[3.5]nonan-7-yl}methyl)-4-(4,4-dimethylpiperidin-1-yl)-2-methyl-5-phenylpyridin-3-yl]-2-(tert-butoxy)acetic acid (0.9 mg, 3%). LCMS (M+H)=548.38. ¹H NMR (500 MHz, methanol-d4) δ 7.60-7.50 (m, 2H), 7.37 (br d, J=6.7 Hz, 1H), 7.25 (br d, J=7.3 Hz, 1H), 6.07 (br s, 1H), 4.16 (br d, J=15.3 Hz, 1H), 3.83 (br d, J=15.3 Hz, 1H), 3.17 (br s, 4H) 2.68 (s, 3H), 2.01-1.91 (m, 2H), 1.91-1.81 (m, 10H), 1.42-1.27 (m, 4H), 1.23 (s, 9H), 0.78 (br s, 6H).

### Example 191

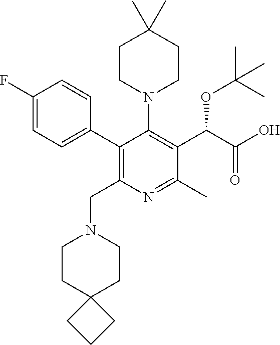 [SMILES: Cc1nc(CN2CCC3(CCC3)CC2)c(-c2ccc(F)cc2)c(N2CCC(C)(C)CC2)c1[C@H](OC(C)(C)C)C(=O)O]

### (2S)-2-[6-({7-Azaspiro[3.5]nonan-7-yl}methyl)-4-(4, 4-dimethylpiperidin-1-yl)-5-(4-fluorophenyl)-2-methylpyridin-3-yl]-2-(tert-butoxy)acetic acid

General method B was followed on a 49 μmol reaction scale using (4-fluorophenyl)boronic acid and (S)-isopropyl 2-(6-(7-azaspiro[3.5]nonan-7-ylmethyl)-5-bromo-4-(4,4-dimethylpiperidin-1-yl)-2-methylpyridin-3-yl)-2-(tert-butoxy)acetate as starting material. The crude material was purified via preparative LC/MS to afford (2S)-2-[6-({7-azaspiro[3.5]nonan-7-yl}methyl)-4-(4,4-dimethylpiperidin-1-yl)-5-(4-fluorophenyl)-2-methylpyridin-3-yl]-2-(tert-butoxy)acetic acid (1.4 mg, 5%). LCMS (M+H)=566.38.

### Example 192

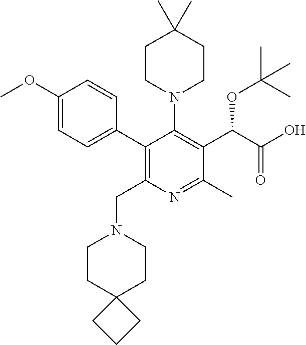 [SMILES: COc1ccc(-c2c(CN3CCC4(CCC4)CC3)nc(C)c([C@H](OC(C)(C)C)C(=O)O)c2N2CCC(C)(C)CC2)cc1]

### (2S)-2-[6-({7-Azaspiro[3.5]nonan-7-yl}methyl)-4-(4, 4-dimethylpiperidin-1-yl)-5-(4-methoxyphenyl)-2-methylpyridin-3-yl]-2-(tert-butoxy)acetic acid

General method B was followed on a 49 μmol reaction scale using (4-methoxyphenyl)boronic acid and (S)-isopropyl 2-(6-(7-azaspiro[3.5]nonan-7-ylmethyl)-5-bromo-4-(4,4-dimethylpiperidin-1-yl)-2-methylpyridin-3-yl)-2-(tert-butoxy)acetate as starting material. The crude material was purified via preparative LC/MS to afford (2S)-2-[6-({7-azaspiro[3.5]nonan-7-yl}methyl)-4-(4,4-dimethylpiperidin-1-yl)-5-(4-methoxyphenyl)-2-methylpyridin-3-yl]-2-(tert-butoxy)acetic acid (4.3 mg, 15%). LCMS (M+H)=578.39. ¹H NMR (500 MHz, methanol-d4) δ 7.25-7.19 (m, 1H), 7.17-7.12 (m, 1H), 7.10-7.03 (m, 2H), 5.72 (s, 1H), 4.04 (d, J=15.3 Hz, 1H), 3.87 (s, 3H), 3.72 (d, J=15.0 Hz, 1H), 3.08-2.98 (m, 4H), 2.65 (d, J=2.1 Hz, 7H), 1.99-1.87 (m, 6H), 1.86-1.80 (m, 8H), 1.33 (br s, 4H), 1.17 (s, 9H), 0.79 (s, 6H).

### Example 193

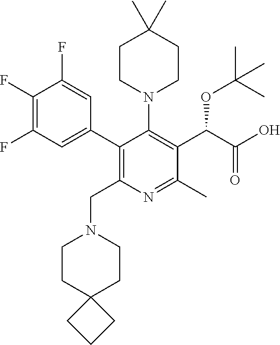 [SMILES: Cc1nc(CN2CCC3(CCC3)CC2)c(-c2cc(F)c(F)c(F)c2)c(N2CCC(C)(C)CC2)c1[C@H](OC(C)(C)C)C(=O)O]

### (2S)-2-[6-({7-Azaspiro[3.5]nonan-7-yl}methyl)-4-(4, 4-dimethylpiperidin-1-yl)-2-methyl-5-(3,4, 5-trifluorophenyl)pyridin-3-yl]-2-(tert-butoxy)acetic acid

To a 14 mL test tube equipped with a stir bar was added (3,4,5-trifluorophenyl)boronic acid (19 mg, 0.111 mmol), (S)-isopropyl 2-(6-(7-azaspiro[3.5]nonan-7-ylmethyl)-5-bromo-4-(4,4-dimethylpiperidin-1-yl)-2-methylpyridin-3-yl)-2-(tert-butoxy)acetate (43.5 mg, 0.072 mmol), tribasic potassium phosphate (46.7 mg, 0.219 mmol), and bis(di-tert-butyl(4-dimethylaminophenyl)phosphine)dichloropalladium(II) (5.2 mg, 7.2 μmol). The test tube was capped with a rubber septum, then was placed under N₂ atmosphere. To the vial was added a degassed (N₂ bubbling for 5 minutes) solution of dioxane (1.125 mL) and water (0.375 mL). The vial was placed in a 60° C. heating block with stirring. After 3 h the temperature was increased to 90° C. After 3 h the reaction mixture was cooled to r.t. and was then diluted with Et₂O (5 mL). The solution was washed with water (5 mL). The isolated organic phase was dried over MgSO₄, then filtered into a 7 mL vial. The volatiles were removed under a N₂ stream. To the vial was added a stir bar and ethanol (1.5 mL), then aq. sodium hydroxide in water (5M, 0.200 mL, 1.00 mmol). The vial was capped, then placed in a 90° C. heating block with stirring for 18 h. The reaction mixture was cooled to r.t., then was filtered through a syringe filter to afford a solution of the crude product. The crude material was purified via preparative LC/MS to afford (2S)-2-[6-({7-azaspiro[3.5]nonan-7-yl}methyl)-4-(4,4-dimethylpiperidin-1-yl)-2-methyl-5-(3,4,5-trifluorophenyl)pyridin-3-yl]-2-(tert-butoxy)acetic acid (8.2 mg, 19%). LCMS (M+H)=602.16. ¹H NMR (500 MHz, methanol-d4) δ 7.32-7.23 (m, 1H), 7.14-7.04 (m, 1H), 5.66 (s, 1H), 4.20 (d, J=15.3 Hz, 1H), 3.79 (d, J=15.3 Hz, 1H), 3.23-2.99 (m, 4H), 2.64 (s, 3H), 2.00-1.80 (m, 11H), 1.76-1.22 (m, 4H), 1.16 (s, 9H), 1.01-0.68 (m, 6H).

### Example 194

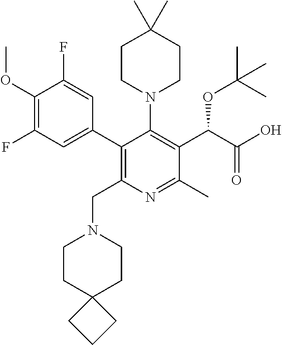 [SMILES: COc1c(F)cc(-c2c(CN3CCC4(CCC4)CC3)nc(C)c([C@H](OC(C)(C)C)C(=O)O)c2N2CCC(C)(C)CC2)cc1F]

### (2S)-2-[6-({7-Azaspiro[3.5]nonan-7-yl}methyl)-5-(3,5-difluoro-4-methoxyphenyl)-4-(4, 4-dimethylpiperidin-1-yl)-2-methylpyridin-3-yl]-2-(tert-butoxy)acetic acid

General method B was followed on a 49 μmol reaction scale using (3,5-difluoro-4-methoxyphenyl)boronic acid and (S)-isopropyl 2-(6-(7-azaspiro[3.5]nonan-7-ylmethyl)-5-bromo-4-(4,4-dimethylpiperidin-1-yl)-2-methylpyridin-3-yl)-2-(tert-butoxy)acetate as starting material. The crude material was purified via preparative LC/MS to afford (2S)-2-[6-({7-azaspiro[3.5]nonan-7-yl}methyl)-5-(3,5-difluoro-4-methoxyphenyl)-4-(4,4-dimethylpiperidin-1-yl)-2-methylpyridin-3-yl]-2-(tert-butoxy)acetic acid (3 mg, 10%). LCMS (M+H)=614.15. ¹H NMR (500 MHz, methanol-d4) δ 7.12 (br d, J=10.7 Hz, 1H), 6.96 (br d, J=11.0 Hz, 1H), 5.69 (s, 1H), 4.23 (d, J=15.3 Hz, 1H), 4.06 (s, 3H), 3.83 (d, J=15.3 Hz, 1H), 3.24-3.01 (m, 4H), 2.66 (s, 3H), 2.01-1.83 (m, 10H), 1.53-1.28 (m, 2H), 1.18 (s, 9H), 0.98-0.72 (m, 6H).

### Example 195

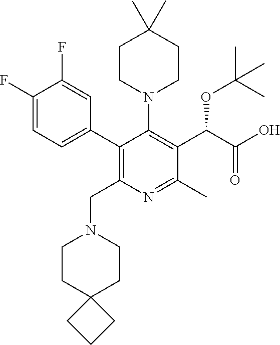 [SMILES: Cc1nc(CN2CCC3(CCC3)CC2)c(-c2ccc(F)c(F)c2)c(N2CCC(C)(C)CC2)c1[C@H](OC(C)(C)C)C(=O)O]

### (2S)-2-[6-({7-Azaspiro[3.5]nonan-7-yl}methyl)-5-(3, 4-difluorophenyl)-4-(4, 4-dimethylpiperidin-1-yl)-2-methylpyridin-3-yl]-2-(tert-butoxy)acetic acid

General method B was followed on a 49 μmol reaction scale using (3,4-difluorophenyl)boronic acid and (S)-isopropyl 2-(6-(7-azaspiro[3.5]nonan-7-ylmethyl)-5-bromo-4-(4,4-dimethylpiperidin-1-yl)-2-methylpyridin-3-yl)-2-(tert-butoxy)acetate as starting material. The crude material was purified via preparative LC/MS to afford (2S)-2-[6-({7-azaspiro[3.5]nonan-7-yl}methyl)-5-(3,4-difluorophenyl)-4-(4,4-dimethylpiperidin-1-yl)-2-methylpyridin-3-yl]-2-(tert-butoxy)acetic acid (2.5 mg, 9%). LCMS (M+H)=584.17. Ratio of atropisomers is 1.0:1.0 as determined by ¹H NMR (500 MHz, methanol-d4) δ 3.79 (d, J=8.5 Hz, 1H), 3.78-3.74 (m, 1H).

¹H NMR (500 MHz, methanol-d4) δ 7.53-7.38 (m, 1.5H), 7.29-7.18 (m, 1H), 7.11-7.05 (m, 0.5H), 5.70 (d, J=2.4 Hz, 1H), 4.19 (dd, J=15.3, 11.6 Hz, 1H), 3.81-3.73 (m, 1H), 3.23-3.02 (m, 4H), 2.68 (s, 2H), 2.66 (s, 3H), 1.99-1.81 (m, 12H), 1.48-1.29 (m, 2H), 1.19 (d, J=1.2 Hz, 9H), 0.96-0.68 (m, 6H).

### Example 196

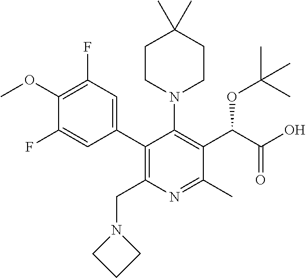 [SMILES: COc1c(F)cc(-c2c(CN3CCC3)nc(C)c([C@H](OC(C)(C)C)C(=O)O)c2N2CCC(C)(C)CC2)cc1F]

### (2S)-2-{6-[(Azetidin-1-yl)methyl]-5-(3, 5-difluoro-4-methoxyphenyl)-4-(4, 4-dimethylpiperidin-1-yl)-2-methylpyridin-3-yl}-2-(tert-butoxy)acetic acid

General method A was followed on a 46 μmol reaction scale using azetidine and (S)-isopropyl 2-(tert-butoxy)-2-(5-(3,5-difluoro-4-methoxyphenyl)-4-(4,4-dimethylpiperidin-1-yl)-6-formyl-2-methylpyridin-3-yl)acetate as starting material. The crude material was purified via preparative LC/MS to afford (2S)-2-{6-[(azetidin-1-yl)methyl]-5-(3,5-difluoro-4-methoxyphenyl)-4-(4,4-dimethylpiperidin-1-yl)-2-methylpyridin-3-yl}-2-(tert-butoxy)acetic acid (6 mg, 24%). LCMS (M+H)=546.31. ¹H NMR (500 MHz, methanol-d4) δ 7.11-7.05 (m, 1H), 6.93 (br d, J=11.0 Hz, 1H), 5.69 (s, 1H), 4.22-4.12 (m, 4H), 4.06 (s, 3H), 2.66 (s, 3H), 2.55-2.47 (m, 2H), 1.50-1.24 (m, 4H), 1.18 (s, 9H), 0.85 (br s, 6H).

### Example 197

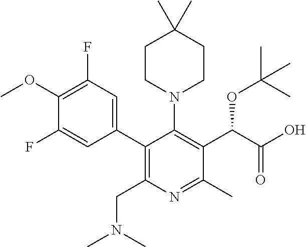 [SMILES: COc1c(F)cc(-c2c(CN(C)C)nc(C)c([C@H](OC(C)(C)C)C(=O)O)c2N2CCC(C)(C)CC2)cc1F]

### (2S)-2-(tert-Butoxy)-2-[5-(3, 5-difluoro-4-methoxyphenyl)-6-[(dimethylamino)methyl]-4-(4, 4-dimethylpiperidin-1-yl)-2-methylpyridin-3-yl]acetic acid

General method A was followed on a 46 μmol reaction scale using dimethylamine in MeOH (2M) and (S)-isopropyl 2-(tert-butoxy)-2-(5-(3,5-difluoro-4-methoxyphenyl)-4-(4,4-dimethylpiperidin-1-yl)-6-formyl-2-methylpyridin-3-yl)acetate as starting material. The crude material was purified via preparative LC/MS to afford (2S)-2-(tert-butoxy)-2-[5-(3,5-difluoro-4-methoxyphenyl)-6-[(dimethylamino)methyl]-4-(4,4-dimethylpiperidin-1-yl)-2-methylpyridin-3-yl]acetic acid (10.7 mg, 44%). LCMS (M+H)=534.29. ¹H NMR (500 MHz, methanol-d4) δ 7.11-7.04 (m, 1H), 6.95 (br d, J=11.3 Hz, 1H), 5.71 (s, 1H), 4.22 (d, J=14.6 Hz, 1H), 4.06 (s, 3H), 3.84 (d, J=14.6 Hz, 1H), 2.82 (s, 6H), 2.68 (s, 3H), 1.38 (br d, J=2.7 Hz, 4H), 1.19 (s, 9H), 0.85 (s, 6H).

### Example 198

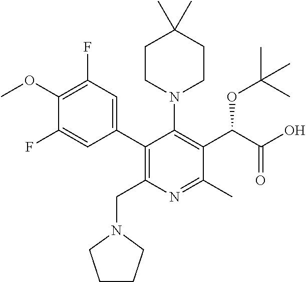 [SMILES: COc1c(F)cc(-c2c(CN3CCCC3)nc(C)c([C@H](OC(C)(C)C)C(=O)O)c2N2CCC(C)(C)CC2)cc1F]

### (2S)-2-(tert-Butoxy)-2-[5-(3, 5-difluoro-4-methoxyphenyl)-4-(4, 4-dimethylpiperidin-1-yl)-2-methyl-6-[(pyrrolidin-1-yl)methyl]pyridin-3-yl]acetic acid

General method A was followed on a 46 μmol reaction scale using pyrrolidine and (S)-isopropyl 2-(tert-butoxy)-2-(5-(3,5-difluoro-4-methoxyphenyl)-4-(4,4-dimethylpiperidin-1-yl)-6-formyl-2-methylpyridin-3-yl)acetate as starting material. The crude material was purified via preparative LC/MS to afford (2S)-2-(tert-butoxy)-2-[5-(3,5-difluoro-4-methoxyphenyl)-4-(4,4-dimethylpiperidin-1-yl)-2-methyl-6-[(pyrrolidin-1-yl)methyl]pyridin-3-yl]acetic acid (8.6 mg, 33%). LCMS (M+H)=560.29. ¹H NMR (500 MHz, methanol-d4) δ 7.09 (br d, J=10.7 Hz, 1H), 6.95 (br d, J=11.3 Hz, 1H), 5.72 (s, 1H), 4.37 (d, J=15.3 Hz, 1H), 4.06 (s, 3H), 3.94 (d, J=15.0 Hz, 1H), 2.67 (s, 3H), 2.07 (dt, J=6.9, 3.4 Hz, 4H), 1.43-1.30 (m, 4H), 1.19 (s, 9H), 0.85 (br s, 6H).

### Example 199

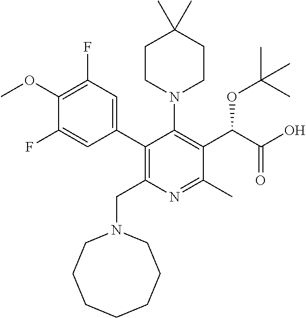 [SMILES: COc1c(F)cc(-c2c(CN3CCCCCCC3)nc(C)c([C@H](OC(C)(C)C)C(=O)O)c2N2CCC(C)(C)CC2)cc1F]

### (2S)-2-{6-[(Azocan-1-yl)methyl]-5-(3, 5-difluoro-4-methoxyphenyl)-4-(4, 4-dimethylpiperidin-1-yl)-2-methylpyridin-3-yl}-2-(tert-butoxy)acetic acid

General method A was followed on a 46 μmol reaction scale using azocane and (S)-isopropyl 2-(tert-butoxy)-2-(5-(3,5-difluoro-4-methoxyphenyl)-4-(4,4-dimethylpiperidin-1-yl)-6-formyl-2-methylpyridin-3-yl)acetate as starting material. The crude material was purified via preparative LC/MS to afford (2S)-2-{6-[(azocan-1-yl)methyl]-5-(3,5-difluoro-4-methoxyphenyl)-4-(4,4-dimethylpiperidin-1-yl)-2-methylpyridin-3-yl}-2-(tert-butoxy)acetic acid (9.6 mg, 35%). LCMS (M+H)=602.33.

¹H NMR (500 MHz, methanol-d4) δ 7.11 (br d, J=10.7 Hz, 1H), 6.96 (br d, J=11.0 Hz, 1H), 5.69 (s, 1H), 4.24 (br d, J=14.3 Hz, 1H), 4.06 (s, 3H), 3.91 (d, J=15.3 Hz, 1H), 3.30-3.22 (m, 4H), 2.68 (s, 3H), 1.91 (br s, 4H), 1.77 (br s, 6H), 1.44-1.33 (m, 4H), 1.19 (s, 9H), 0.86 (s, 6H).

### Example 200

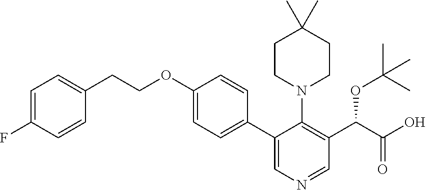 [SMILES: CC1(C)CCN(c2c(-c3ccc(OCCc4ccc(F)cc4)cc3)cncc2[C@H](OC(C)(C)C)C(=O)O)CC1]

### (S)-2-(tert-Butoxy)-2-(4-(4, 4-dimethylpiperidin-1-yl)-5-(4-(4-fluorophenethoxy)phenyl)pyridin-3-yl)acetic acid

To a solution of ethyl-(S)-2-(tert-butoxy)-2-(2-chloro-4-(4,4-dimethylpiperidin-1-yl)-5-(4-(4-fluorophenethoxy)phenyl)pyridin-3-yl)acetate (14.7 mg, 0.025 mmol) in MeOH (5 mL) in reaction vial was added palladium hydroxide on carbon (8.0 mg, 0.011 mmol). The reaction was flushed with nitrogen, capped, purged with H₂ and stirred at room temp for 3 h under an atmosphere (balloon) of H₂. The reaction was then treated with acetic acid (33.4 μL, 0.583 mmol), purged with H₂ and heated at 50° C. for 18 h. The catalyst was filtered off through a 45 m frit and the resulting solution was transferred to a pressure bottle. The reaction was charged with a mixture of palladium hydroxide (9 mg) and Pd/C (6 mg), purged with H2 gas, capped and heated at in a 65° C. for 18 h. The catalyst was filtered off through a 45 m frit and the solvent was removed under a gentle stream of nitrogen. The residue was dissolved in EtOH (3 mL), treated with 10 M sodium hydroxide (100 μL, 1.000 mmol) and heated at 105° C. for 4.5 h. The crude material was purified via preparative LC/MS to afford (S)-2-(tert-butoxy)-2-(4-(4,4-dimethylpiperidin-1-yl)-5-(4-(4-fluorophenethoxy)phenyl)pyridin-3-yl)acetic acid, 5.0 mg), (38%). LCMS (M+1)=535.3.

### Example 201

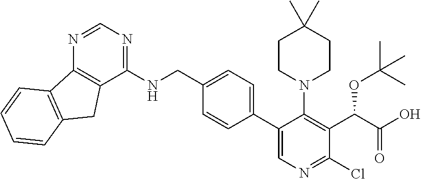 [SMILES: CC1(C)CCN(c2c(-c3ccc(CNc4ncnc5c4Cc4ccccc4-5)cc3)cnc(Cl)c2[C@H](OC(C)(C)C)C(=O)O)CC1]

### (S)-2-(5-(4-((Benzofuro[3, 2-d]pyrimidin-4-ylamino)methyl)phenyl)-2-chloro-4-(4, 4-dimethylpiperidin-1-yl)pyridin-3-yl)-2-(tert-butoxy)acetic acid

To a dry reaction vial under NITROGEN was added ethyl-(S)-2-(5-bromo-2-chloro-4-(4,4-dimethylpiperidin-1-yl)pyridin-3-yl)-2-(tert-butoxy)acetate (32.6 mg, 0.071 mmol), N-(4-(4,4,5,5-tetramethyl-1,3,2-dioxaborolan-2-yl)benzyl)benzofuro[3,2-d]pyrimidin-4-amine (34 mg, 0.085 mmol) and THF (4 mL). The reaction was flushed with argon, treated with 0.5 M potassium phosphate tribasic (490 μL, 0.245 mmol), followed by 2^{nd} generation X-phos precatalyst (4 mg, 5.08 μmol), capped and stirred at room temp for 18 h. The solvent was removed under a gentle stream of air and the crude product was dissolved in dichloromethane (4 mL). The resulting solution was treated with QuadraSil AP, loading 1.5-2 mmol/gram (36 mg), stirred at room temp for 10 min, filtered through a 45μ frit and the solvent was removed under a gentle stream of air. The residue was dissolved in EtOH (4 mL), treated with 10 M sodium hydroxide (110 μL, 1.100 mmol) and heated at 105° C. for 3.5 h. The crude material was purified via preparative LC/MS to afford (S)-2-(5-(4-((benzofuro[3,2-d]pyrimidin-4-ylamino)methyl)phenyl)-2-chloro-4-(4,4-dimethylpiperidin-1-yl)pyridin-3-yl)-2-(tert-butoxy)acetic acid, 8.4 mg (19%). LCMS (M+1)=628.2.

### Example 202

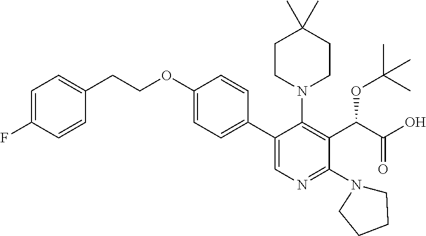 [SMILES: CC1(C)CCN(c2c(-c3ccc(OCCc4ccc(F)cc4)cc3)cnc(N3CCCC3)c2[C@H](OC(C)(C)C)C(=O)O)CC1]

### (S)-2-(tert-Butoxy)-2-(4-(4, 4-dimethylpiperidin-1-yl)-5-(4-(4-fluorophenethoxy)phenyl)-2-(pyrrolidin-1-yl)pyridin-3-yl)acetic acid

To a dry microwave vial under nitrogen was added ethyl-(S)-2-(tert-butoxy)-2-(2-chloro-4-(4,4-dimethylpiperidin-1-yl)-5-(4-(4-fluorophenethoxy)phenyl)pyridin-3-yl)acetate (9.1 mg, 0.015 mmol), NMP (1.0 mL), pyrrolidine (20 μL, 0.242 mmol) and N,N-diisopropylethylamine (40 μL, 0.229 mmol). The reaction was capped and heated in a microwave reactor at 140-200° C. for 14 h. The solvent was removed under a gentle stream of nitrogen and the residue was redissolved in EtOH (3 mL). The resulting solution was treated with 10 M sodium hydroxide in water (85 μl, 0.850 mmol) and heated at 105° C. for 18 h. The crude material was purified via preparative LC/MS to afford (S)-2-(tert-butoxy)-2-(4-(4,4-dimethylpiperidin-1-yl)-5-(4-(4-fluorophenethoxy)phenyl)-2-(pyrrolidin-1-yl)pyridin-3-yl)acetic acid, 7.0 mg (72%). LCMS (M+1)=604.3.

### Example 203

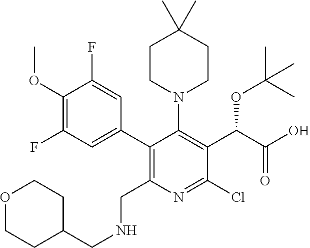 [SMILES: COc1c(F)cc(-c2c(CNCC3CCOCC3)nc(Cl)c([C@H](OC(C)(C)C)C(=O)O)c2N2CCC(C)(C)CC2)cc1F]

### (S)-2-(tert-Butoxy)-2-(2-chloro-5-(3, 5-difluoro-4-methoxyphenyl)-4-(4, 4-dimethylpiperidin-1-yl)-6-((((tetrahydro-2H-pyran-4-yl)methyl)amino)methyl)pyridin-3-yl)acetic acid

To a dry vial under nitrogen was added ethyl-(S)-2-(tert-butoxy)-2-(2-chloro-5-(3,5-difluoro-4-methoxyphenyl)-4-(4,4-dimethylpiperidin-1-yl)-6-formylpyridin-3-yl)acetate (30 mg, 0.054 mmol), 4-aminomethyltetrahydropyran (20.25 mg, 0.176 mmol), ClCH₂CH₂Cl (1.25 mL), acetic acid (11.5 μl, 0.201 mmol) and several pieces of 4 A mol sieves. The reaction was stirred at room temp for 10 min, treated with ethanol (0.625 mL) and stirred at room temp for 3 h. The reaction was then treated (slowly) with sodium cyanoborohydride, 1.0M in THF (217 μL, 0.217 mmol). After the addition was complete, the reaction was stirred at room temp for 5 min, then the solvent was removed under a gentle stream of nitrogen. The residue was dissolved in EtOH (2.5 mL), treated with 10 M sodium hydroxide (115 μL, 1.15 mmol) and heated at 105° C. for 2.5 h. The crude material was purified via preparative LC/MS to afford (S)-2-(tert-butoxy)-2-(2-chloro-5-(3,5-difluoro-4-methoxyphenyl)-4-(4,4-dimethylpiperidin-1-yl)-6-((((tetrahydro-2H-pyran-4-yl)methyl)amino)methyl)pyridin-3-yl)acetic acid, 20 mg (58%). LCMS (M+1)=624.2.

### Example 204

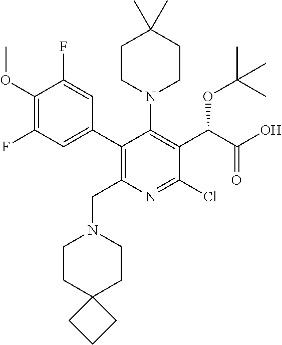 [SMILES: COc1c(F)cc(-c2c(CN3CCC4(CCC4)CC3)nc(Cl)c([C@H](OC(C)(C)C)C(=O)O)c2N2CCC(C)(C)CC2)cc1F]

### (S)-2-(6-((7-Azaspiro[3.5]nonan-7-yl)methyl)-2-chloro-5-(3, 5-difluoro-4-methoxyphenyl)-4-(4, 4-dimethylpiperidin-1-yl)pyridin-3-yl)pyridin-3-yl)-2-(tert-butoxy)acetic acid

To a dry vial under nitrogen was added ethyl-(S)-2-(tert-butoxy)-2-(2-chloro-5-(3,5-difluoro-4-methoxyphenyl)-4-(4,4-dimethylpiperidin-1-yl)-6-formylpyridin-3-yl)acetate (30 mg, 0.054 mmol), 7-azaspiro[3.5]nonane (22 mg, 0.176 mmol), ClCH₂CH₂Cl (1.25 mL), acetic acid (11.5 μl, 0.201 mmol) and several pieces of 4 A mol sieves. The reaction was stirred at room temp for 10 min, treated with ethanol (0.625 mL) and stirred at room temp for 70 min. The reaction was then treated (slowly) with sodium cyanoborohydride, 1.0M in THF (217 μL, 0.217 mmol). After the addition was complete, the reaction was stirred at room temp for 5 min, then the solvent was removed under a gentle stream of nitrogen. The residue was dissolved in EtOH (2.5 mL), treated with 10 M sodium hydroxide (70 μL, 0.70 mmol) and heated at 105° C. for 7 h. The crude material was purified via preparative LC/MS to afford (S)-2-(6-((7-azaspiro[3.5]nonan-7-yl)methyl)-2-chloro-5-(3,5-difluoro-4-methoxyphenyl)-4-(4,4-dimethylpiperidin-1-yl)pyridin-3-yl)-2-(tert-butoxy)acetic acid, 8.2 mg (23%). LCMS (M+1)=634.2.

Also isolated from this reaction was

### Example 205

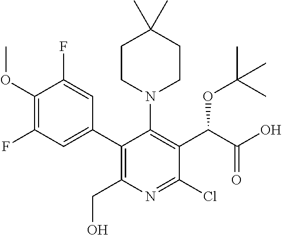 [SMILES: COc1c(F)cc(-c2c(CO)nc(Cl)c([C@H](OC(C)(C)C)C(=O)O)c2N2CCC(C)(C)CC2)cc1F]

(S)-2-(tert-Butoxy)-2-(2-chloro-5-(3, 5-difluoro-4-methoxyphenyl)-4-(4, 4-dimethylpiperidin-1-yl)-6-(hydroxymethyl)pyridin-3-yl)acetic acid, 2.5 mg (8%). LCMS (M+1)=527.1.

### Example 206

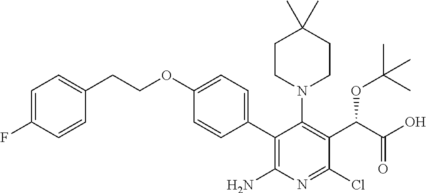 [SMILES: CC1(C)CCN(c2c(-c3ccc(OCCc4ccc(F)cc4)cc3)c(N)nc(Cl)c2[C@H](OC(C)(C)C)C(=O)O)CC1]

### (S)-2-(6-Amino-2-chloro-4-(4, 4-dimethylpiperidin-1-yl)-5-(4-(4-fluorophenethoxy)phenyl)pyridin-3-yl)-2-(tert-butoxy)acetic acid

To a reaction vial under nitrogen was added ethyl (S)-2-(6-amino-2-chloro-4-(4,4-dimethylpiperidin-1-yl)-5-(4-(4-fluorophenethoxy)phenyl)pyridin-3-yl)-2-(tert-butoxy)acetate (34.9 mg, 0.057 mmol) and ethanol (4 mL). The reaction was treated with 10 M sodium hydroxide (70 μL, 0.700 mmol) and heated at 105° C. for 3.5 h. The crude material was purified via preparative LC/MS to afford (S)-2-(6-amino-2-chloro-4-(4,4-dimethylpiperidin-1-yl)-5-(4-(4-fluorophenethoxy)phenyl)pyridin-3-yl)-2-(tert-butoxy)acetic acid, 15.3 mg (46%). LCMS (M+1)=584.2.

### Example 207

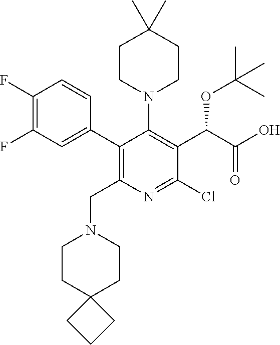 [SMILES: CC1(C)CCN(c2c(-c3ccc(F)c(F)c3)c(CN3CCC4(CCC4)CC3)nc(Cl)c2[C@H](OC(C)(C)C)C(=O)O)CC1]

### (S)-2-(6-((7-Azaspiro[3.5]nonan-7-yl)methyl)-2-chloro-5-(3, 4-difluorophenyl)-4-(4, 4-dimethylpiperidin-1-yl)pyridin-3-yl)-2-(tert-butoxy)acetic acid

To a solution of ethyl-(S)-2-(tert-butoxy)-2-(2-chloro-5-(3,4-difluorophenyl)-4-(4,4-dimethylpiperidin-1-yl)-6-formylpyridin-3-yl)acetate (60.8 mg, 0.116 mmol) in a mixture of ClCH₂CH₂Cl (3.0 mL) and EtOH (2.0 mL) was added 7-azaspiro[3.5]nonane (55 mg, 0.439 mmol), acetic acid (28 μL, 0.489 mmol), and several pieces of 4 A mol sieves. The reaction was stirred at room temp, for 2 h, then treated (slowly) with sodium cyanoborohydride, 1.0 M in THF (410 μL, 0.410 mmol). After the addition was complete, the reaction was stirred at room temp for 5 min, then the solvent was removed under a gentle stream of nitrogen. The residue was dissolved in EtOH (4.5 mL), treated with 10 M sodium hydroxide (180 μL, 1.80 mmol) and heated at 105° C. for 3.5 h. The crude material was purified via preparative LC/MS to afford (S)-2-(6-((7-azaspiro[3.5]nonan-7-yl)methyl)-2-chloro-5-(3,4-difluorophenyl)-4-(4,4-dimethylpiperidin-1-yl)pyridin-3-yl)-2-(tert-butoxy)acetic acid, 9.7 mg, (12%). LCMS (M+1)=604.3.

Also isolated from this reaction is

### Example 208

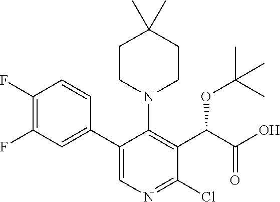 [SMILES: CC1(C)CCN(c2c(-c3ccc(F)c(F)c3)cnc(Cl)c2[C@H](OC(C)(C)C)C(=O)O)CC1]

(S)-2-(tert-Butoxy)-2-(2-chloro-5-(3, 4-difluorophenyl)-4-(4, 4-dimethylpiperidin-1-yl)pyridin-3-yl)acetic acid (1.7 mg, 3%). LCMS (M+1)=467.2.

### Example 209

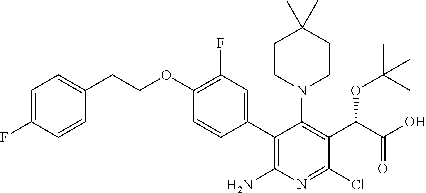 [SMILES: CC1(C)CCN(c2c(-c3ccc(OCCc4ccc(F)cc4)c(F)c3)c(N)nc(Cl)c2[C@H](OC(C)(C)C)C(=O)O)CC1]

### (S)-2-(6-Amino-2-chloro-4-(4, 4-dimethylpiperidin-1-yl)-5-(3-fluoro-4-(4-fluorophenethoxy)phenyl)pyridin-3-yl)-2-(tert-butoxy)acetic acid

To a dry reaction vial under nitrogen was added ethyl-(S)-2-(6-amino-5-bromo-2-chloro-4-(4,4-dimethylpiperidin-1-yl)pyridin-3-yl)-2-(tert-butoxy)acetate (34 mg, 0.071 mmol), 2-(3-fluoro-4-(4-fluorophenethoxy)phenyl)-4,4,5,5-tetramethyl-1,3,2-dioxaborolane (50 mg, 0.139 mmol) and THF (4 mL). The reaction was flushed with argon, treated with 0.5 M potassium phosphate tribasic (0.6 mL, 0.300 mmol), followed by 2nd generation X-phos precatalyst (5.2 mg, 6.61 μmol), capped and stirred at room temp for 18 h. The reaction was dissolved in EtOAc, extracted with water, brine, dried over Na₂SO₄ and concentrated. The crude material was purified via silica gel chromatography (12 g SiO₂ column, dichloromethane:EtOAc 100:0->65:35) to afford desired ester 35.2 mg (78%). This intermediate was dissolved in ethanol (4.5 mL), treated with 10 M sodium hydroxide (75 μL, 0.750 mmol) and heated at 105° C. for 3.5 h. The crude material was purified via preparative LC/MS to afford (S)-2-(6-amino-2-chloro-4-(4,4-dimethylpiperidin-1-yl)-5-(3-fluoro-4-(4-fluorophenethoxy)phenyl)pyridin-3-yl)-2-(tert-butoxy)acetic acid, 29.8 mg (88%). LCMS (M+1)=602.2.

### Example 210

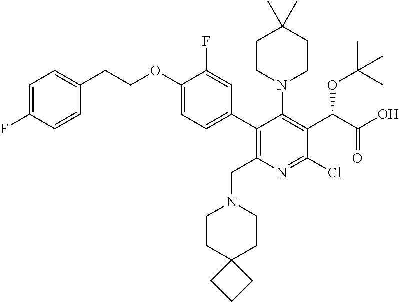 [SMILES: CC1(C)CCN(c2c(-c3ccc(OCCc4ccc(F)cc4)c(F)c3)c(CN3CCC4(CCC4)CC3)nc(Cl)c2[C@H](OC(C)(C)C)C(=O)O)CC1]

### (S)-2-(6-((7-Azaspiro[3.5]nonan-7-yl)methyl)-2-chloro-4-(4, 4-dimethylpiperidin-1-yl)-5-(3-fluoro-4-(4-fluorophenethoxy)phenyl)pyridin-3-yl)-2-(tert-butoxy)acetic acid

To a dry reaction vial under nitrogen was added ethyl-(S)-2-(tert-butoxy)-2-(2-chloro-4-(4,4-dimethylpiperidin-1-yl)-5-(3-fluoro-4-(4-fluorophenethoxy)phenyl)-6-formylpyridin-3-yl)acetate (67.8 mg, 0.105 mmol), ClCH₂CH₂Cl (2.0 mL), 7-azaspiro[3.5]nonane (51 mg, 0.407 mmol), acetic acid (24 μL, 0.419 mmol) and 4 A mol sieves. The reaction was stirred at room temp for 15 min, then treated with EtOH (1.0 mL). The reaction was stirred at room temp for 90 min, treated (slowly, over 2.5 h) sodium cyanoborohydride, 1.0M in THF (420 μL, 0.420 mmol). After the addition was complete, the reaction was allowed to stirred at room temp for 20 min, then the solvent was removed under a gentle stream of nitrogen overnight. The residue was dissolved in EtOH (4 mL), treated with 10 M sodium hydroxide (160 μL, 1.600 mmol) and heated at 105° C. for 4.5 h. The crude material was purified via preparative LC/MS to afford (S)-2-(6-((7-azaspiro[3.5]nonan-7-yl)methyl)-2-chloro-4-(4,4-dimethylpiperidin-1-yl)-5-(3-fluoro-4-(4-fluorophenethoxy)phenyl)pyridin-3-yl)-2-(tert-butoxy)acetic acid, 17.3 mg (23%). LCMS (M+1)=724.3.

### Example 211

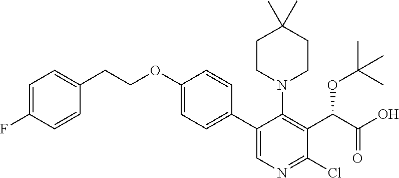 [SMILES: CC1(C)CCN(c2c(-c3ccc(OCCc4ccc(F)cc4)cc3)cnc(Cl)c2[C@H](OC(C)(C)C)C(=O)O)CC1]

### (S)-2-(tert-Butoxy)-2-(2-chloro-4-(4, 4-dimethylpiperidin-1-yl)-5-(4-(4-fluorophenethoxy)phenyl)pyridin-3-yl)acetic acid

To a dry reaction vial under nitrogen was added ethyl-(S)-2-(5-bromo-2-chloro-4-(4,4-dimethylpiperidin-1-yl)pyridin-3-yl)-2-(tert-butoxy)acetate (36.2 mg, 0.078 mmol), (4-(4-fluorophenethoxy)phenyl)boronic acid (25.3 mg, 0.097 mmol) and THF (4 mL). The reaction was flushed with argon, treated with 0.5 M potassium phosphate tribasic (450 μl, 0.225 mmol), followed by 2^{nd} generation X-phos precatalyst (5 mg, 6.35 μmol), capped and stirred at room temp for 18 h. The solvent was removed under a gentle stream of air and the crude product was dissolved in dichloromethane (4 mL) and the solvent removed under a gentle stream of air. The residue was dissolved in EtOH (2 mL), stirred at room temp for 1 h, treated with 10 M sodium hydroxide (115 μL, 1.150 mmol) and heated at 105° C. for 35 min. The crude material was purified via preparative LC/MS to afford (S)-2-(tert-butoxy)-2-(2-chloro-4-(4,4-dimethylpiperidin-1-yl)-5-(4-(4-fluorophenethoxy)phenyl)pyridin-3-yl)acetic acid, 34.1 mg (76%). LCMS (M+1)=569.1.

### Example 212

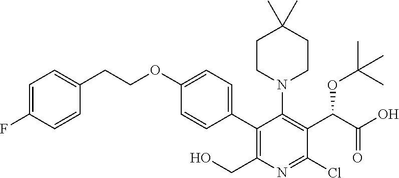 [SMILES: CC1(C)CCN(c2c(-c3ccc(OCCc4ccc(F)cc4)cc3)c(CO)nc(Cl)c2[C@H](OC(C)(C)C)C(=O)O)CC1]

### (S)-2-(tert-Butoxy)-2-(2-chloro-4-(4, 4-dimethylpiperidin-1-yl)-5-(4-(4-fluorophenethoxy)phenyl)-6-(hydroxymethyl)pyridin-3-yl)acetic acid

To a dry reaction vial under nitrogen was added ethyl-(S)-2-(tert-butoxy)-2-(2-chloro-4-(4,4-dimethylpiperidin-1-yl)-5-(4-(4-fluorophenethoxy)phenyl)-6-formylpyridin-3-yl)acetate (40 mg, 0.064 mmol) and EtOH (2 mL). The reaction was treated with sodium borohydride (11 mg, 0.291 mmol), stirred at room temp for 5 min, then then treated with 10 M sodium hydroxide (55 μl, 0.550 mmol) and and heated at 105° C. for 20 min. The reaction was treated with additional 10 M sodium hydroxide (40 μl, 0.400 mmol) and heated at 105° C. for 2 h. The crude material was purified via preparative LC/MS to afford (S)-2-(tert-butoxy)-2-(2-chloro-4-(4,4-dimethylpiperidin-1-yl)-5-(4-(4-fluorophenethoxy)phenyl)-6-(hydroxymethyl)pyridin-3-yl)acetic acid, 20.5 mg (52%). LCMS (M+1)=599.2.

### Example 213

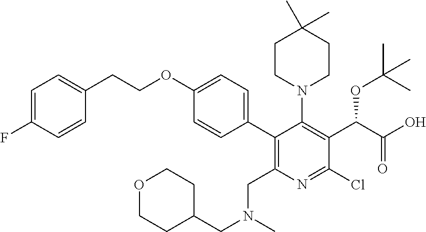 [SMILES: CN(Cc1nc(Cl)c([C@H](OC(C)(C)C)C(=O)O)c(N2CCC(C)(C)CC2)c1-c1ccc(OCCc2ccc(F)cc2)cc1)CC1CCOCC1]

### (S)-2-(tert-Butoxy)-2-(2-chloro-4-(4, 4-dimethylpiperidin-1-yl)-5-(4-(4-fluorophenethoxy)phenyl)-6-((methyl((tetrahydro-2H-pyran-4-yl)methyl)amino)methyl)pyridin-3-yl)acetic acid

To a dry reaction vial under nitrogen was added N-methyl-1-(tetrahydro-2H-pyran-4-yl)methanamine (30 mg, 0.232 mmol), (S)-ethyl 2-(tert-butoxy)-2-(2-chloro-4-(4,4-dimethylpiperidin-1-yl)-5-(4-(4-fluorophenethoxy)phenyl)-6-formylpyridin-3-yl)acetate (40 mg, 0.064 mmol), ClCH₂CH₂Cl (1.5 mL), acetic acid (14 μl, 0.245 mmol) and 4 pieces of activated 4 A sieves. The reaction was flushed with argon, stirred at room temp for 1 min, treated with ethanol (0.5 mL) and stirred at room temp for 1 h. The reaction was then treated (slowly) with sodium cyanoborohydride 1 M in THF (250 μl, 0.250 mmol). After the addition was complete, the solvent was removed under a gentle stream of nitrogen. The residue was dissolved in ethanol (3 mL), treated with 10 M sodium hydroxide (80 μl, 0.800 mmol) and heated at 60 C for 18 h. The reaction was treated with additional 10 M sodium hydroxide (175 μl, 1.75 mmol) and heated at 105° C. for 44 h. The crude material was purified via preparative LC/MS to afford (S)-2-(tert-butoxy)-2-(2-chloro-4-(4,4-dimethylpiperidin-1-yl)-5-(4-(4-fluorophenethoxy)phenyl)-6-((methyl((tetrahydro-2H-pyran-4-yl)methyl)amino)methyl)pyridin-3-yl)acetic acid, 16.1 mg (34%). LCMS (M+1)=710.3.

### Example 214

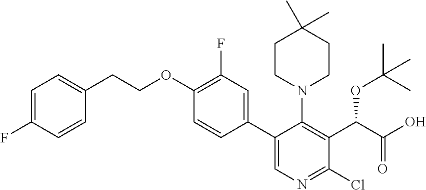 [SMILES: CC1(C)CCN(c2c(-c3ccc(OCCc4ccc(F)cc4)c(F)c3)cnc(Cl)c2[C@H](OC(C)(C)C)C(=O)O)CC1]

### (S)-2-(tert-Butoxy)-2-(2-chloro-4-(4, 4-dimethylpiperidin-1-yl)-5-(3-fluoro-4-(4-fluorophenethoxy)phenyl)pyridin-3-yl)acetic acid

To a dry reaction vial under nitrogen was added (S)-ethyl 2-(tert-butoxy)-2-(2-chloro-4-(4,4-dimethylpiperidin-1-yl)-5-(3-fluoro-4-(4-fluorophenethoxy)phenyl)pyridin-3-yl)acetate (56 mg, 0.091 mmol), ethanol (4 mL) and sodium hydroxide 10 N (90 μl, 0.900 mmol). The reaction was capped and heated at 105 C for 5 h. The crude material was purified via preparative LC/MS to afford (S)-2-(tert-butoxy)-2-(2-chloro-4-(4,4-dimethylpiperidin-1-yl)-5-(3-fluoro-4-(4-fluorophenethoxy)phenyl)pyridin-3-yl)acetic acid, 26.8 mg (50%). LCMS (M+1)=587.2.

### Example 215

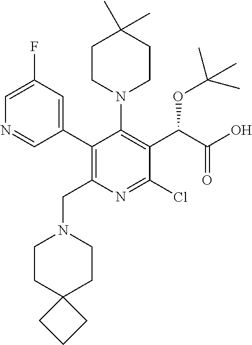 [SMILES: CC1(C)CCN(c2c(-c3cncc(F)c3)c(CN3CCC4(CCC4)CC3)nc(Cl)c2[C@H](OC(C)(C)C)C(=O)O)CC1]

### (S)-2-(2-((7-Azaspiro[3.5]nonan-7-yl)methyl)-6-chloro-4-(4,4-dimethylpiperidin-1-yl)-5′-fluoro-[3, 3′-bipyridin]-5-yl)-2-(tert-butoxy)acetic acid

To a dry reaction vial under nitrogen was added (S)-ethyl 2-(tert-butoxy)-2-(6-chloro-4-(4,4-dimethylpiperidin-1-yl)-5′-fluoro-2-formyl-[3,3′-bipyridin]-5-yl)acetate and ethyl (S)-2-(tert-butoxy)-2-(6-chloro-4-(4,4-dimethylpiperidin-1-yl)-5′-fluoro-[3,3′-bipyridin]-5-yl)acetate (14 mg, 0.028 mmol) ClCH₂CH₂Cl (1.5 mL), 7-azaspiro[3.5]nonane (50 mg, 0.399 mmol), acetic acid (14 μl, 0.245 mmol) and 2 pieces of 4 A mol sieves. The reaction was stirred at room temp for 2 min, treated with ethanol (0.5 mL) and stirred at room temp for 20 min. The reaction was treated (slowly) with sodium cyanoborohydride 1 M in THF (330 μl, 0.330 mmol). After the addition was complete, the solvent was removed under a gentle stream of nitrogen. The residue was dissolved in ethanol 92 mL), treated 10 M sodium hydroxide (110 μl, 1.100 mmol) and heated at 105° C. for 6 h. The reaction was then treated with additional 10 M sodium hydroxide (60 μl, 0.600 mmol) and heated at 105° C. for 5 h. The crude material was purified via preparative LC/MS to afford (S)-2-(2-((7-azaspiro[3.5]nonan-7-yl)methyl)-6-chloro-4-(4,4-dimethylpiperidin-1-yl)-5′-fluoro-[3,3′-bipyridin]-5-yl)-2-(tert-butoxy)acetic acid, 0.6 mg (1.1%). LCMS (M+1)=587.3.

Also isolated from this reaction is

### Example 216

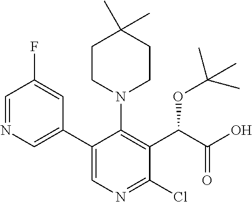 [SMILES: CC1(C)CCN(c2c(-c3cncc(F)c3)cnc(Cl)c2[C@H](OC(C)(C)C)C(=O)O)CC1]

(S)-2-(tert-butoxy)-2-(6-chloro-4-(4, 4-dimethylpiperidin-1-yl)-5′-fluoro-[3, 3′-bipyridin]-5-yl)acetic acid, 0.3 mg (0.7%). LCMS (M+1)=450.1

### Example 217

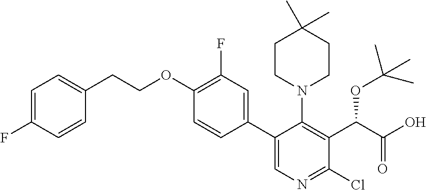 [SMILES: CC1(C)CCN(c2c(-c3ccc(OCCc4ccc(F)cc4)c(F)c3)cnc(Cl)c2[C@H](OC(C)(C)C)C(=O)O)CC1]

### (S)-2-(tert-Butoxy)-2-(2-cyano-4-(4, 4-dimethylpiperidin-1-yl)-5-(3-fluoro-4-(4-fluorophenethoxy)phenyl)pyridin-3-yl)acetic acid

To a dry reaction vial under nitrogen was added (S)-ethyl 2-(tert-butoxy)-2-(2-cyano-4-(4,4-dimethylpiperidin-1-yl)-5-(3-fluoro-4-(4-fluorophenethoxy)phenyl)pyridin-3-yl)acetate (29.1 mg, 0.048 mmol), ethanol (2 mL) and lithium hydroxide 2 M (50.5 μL, 0.101 mmol). The reaction was flushed with nitrogen and heated at 100° C. for 35 min. The crude material was purified via preparative LC/MS to afford (S)-2-(tert-butoxy)-2-(2-cyano-4-(4,4-dimethylpiperidin-1-yl)-5-(3-fluoro-4-(4-fluorophenethoxy)phenyl)pyridin-3-yl)acetic acid, 16.2 mg (56%). LCMS (M+1)=578.2.

### Example 218

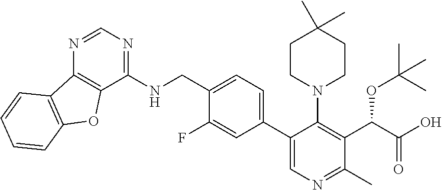 [SMILES: Cc1ncc(-c2ccc(CNc3ncnc4c3oc3ccccc34)c(F)c2)c(N2CCC(C)(C)CC2)c1[C@H](OC(C)(C)C)C(=O)O]

### (S)-2-(5-(4-((Benzofuro[3, 2-d]pyrimidin-4-ylamino)methyl)-3-fluorophenyl)-4-(4, 4-dimethylpiperidin-1-yl)-2-methylpyridin-3-yl)-2-(tert-butoxy)acetic acid

In a pressure vial equipped with a magnetic stirring bar was added (S)-isopropyl 2-(5-bromo-4-(4,4-dimethylpiperidin-1-yl)-2-methylpyridin-3-yl)-2-(tert-butoxy)acetate (66 mg, 0.145 mmol) and N-(2-fluoro-4-(4,4,5,5-tetramethyl-1,3,2-dioxaborolan-2-yl)benzyl)benzofuro[3,2-d]pyrimidin-4-amine (91 mg, 0.217 mmol). The solids were suspended in distilled THF (5 mL). The mixture was treated with 0.5M K₃PO₄ (1.014 mL, 0.507 mmol) and X-Phos precatalyst G2 (9.69 mg, 0.012 mmol). Argon was streamed over and bubbled into the mixture for 5 minutes with sonication. The vial was capped and stirred at RT for 16 hours. LC/MS showed the desired ester intermediate. Removed water layer and then removed solvent under vacuum. The solids were suspended in ethanol (2 mL) within a pressure. 10 M sodium hydroxide (0.130 mL, 1.299 mmol) was added to the mixture, and the vial capped and heated to 80° C. for 16 hours. LC/MS showed some of the hydrolysis product. The reaction mixture was cooled, then filtered and was purified via preparative LC/MS to give 9.1 mg (10%) of the desired compound. LCMS (M+1)=626.3. ¹H NMR (500 MHz, DMSO-d₆) δ 8.57 (br t, J=6.1 Hz, 1H), 8.49 (s, 1H), 8.10 (d, J=7.7 Hz, 1H), 8.05 (s, 1H), 7.80 (d, J=8.4 Hz, 1H), 7.71 (t, J=7.5 Hz, 1H), 7.52 (td, J=7.6, 2.8 Hz, 2H), 7.18 (br d, J=10.6 Hz, 1H), 7.09 (d, J=8.1 Hz, 1H), 5.82 (s, 1H), 4.94-4.78 (m, 2H), 2.58-2.52 (m, 2H), 1.92 (s, 2H), 1.37-1.20 (m, 5H), 1.20-0.98 (m, 12H), 0.87-0.63 (m, 6H).

### Example 219

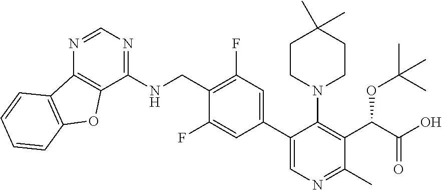 [SMILES: Cc1ncc(-c2cc(F)c(CNc3ncnc4c3oc3ccccc34)c(F)c2)c(N2CCC(C)(C)CC2)c1[C@H](OC(C)(C)C)C(=O)O]

### (S)-2-(5-(4-((Benzofuro[3, 2-d]pyrimidin-4-ylamino)methyl)-3, 5-difluorophenyl)-4-(4, 4-dimethylpiperidin-1-yl)-2-methylpyridin-3-yl)-2-(tert-butoxy)acetic acid

In a pressure vial equipped with a magnetic stirring bar was added (S)-isopropyl 2-(5-bromo-4-(4,4-dimethylpiperidin-1-yl)-2-methylpyridin-3-yl)-2-(tert-butoxy)acetate (50 mg, 0.110 mmol) and N-(2,6-difluoro-4-(4,4,5,5-tetramethyl-1,3,2-dioxaborolan-2-yl)benzyl)benzofuro[3,2-d]pyrimidin-4-amine (72.0 mg, 0.165 mmol). The solids were suspended in distilled THF (5 mL). The mixture was treated with 0.5M K₃PO₄ (0.769 mL, 0.384 mmol) and X-Phos precatalyst G2 (7.34 mg, 9.33 μmol). Argon was streamed over and bubbled into the mixture for 5 minutes with sonication. The vial was capped and stirred at RT for 48 hours. LC/MS showed the desired ester intermediate. Removed water layer and then removed solvent under vacuum. The remaining residue was taken up in Ethanol (2 mL). 10M sodium hydroxide (0.110 mL, 1.098 mmol) was added to the mixture, and the vial capped and heated to 80° C. for 16 hours. LC/MS showed the hydrolysis product. The reaction mixture was purified via preparative LC/MS to give 36.6 mg (51%) of the desired compound. LCMS (M+1)=644.2. ¹H NMR (500 MHz, DMSO-d₆) δ 8.51-8.42 (m, 2H), 8.09 (d, J=7.6 Hz, 1H), 8.07 (s, 1H), 7.77 (d, J=8.1 Hz, 1H), 7.69 (t, J=7.7 Hz, 1H), 7.50 (t, J=7.2 Hz, 1H), 7.06 (d, J=8.8 Hz, 2H), 5.79 (s, 1H), 4.88 (d, J=5.5 Hz, 2H), 2.55 (s, 3H), 1.26 (br d, J=13.9 Hz, 4H), 1.12 (s, 12H), 0.76 (br s, 3H), 0.72 (br s, 5H).

### Example 220

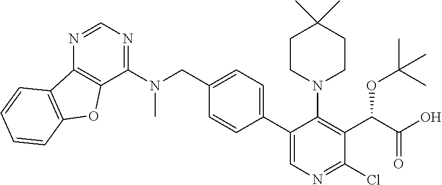 [SMILES: CN(Cc1ccc(-c2cnc(Cl)c([C@H](OC(C)(C)C)C(=O)O)c2N2CCC(C)(C)CC2)cc1)c1ncnc2c1oc1ccccc12]

### (S)-2-(5-(4-((Benzofuro[3, 2-d]pyrimidin-4-yl(methyl)amino)methyl)phenyl)-2-chloro-4-(4, 4-dimethylpiperidin-1-yl)pyridin-3-yl)-2-(tert-butoxy)acetic acid

In a vial equipped with a magnetic stirring bar was added (S)-ethyl 2-(5-bromo-2-chloro-4-(4,4-dimethylpiperidin-1-yl)pyridin-3-yl)-2-(tert-butoxy)acetate (31 mg, 0.067 mmol) and N-methyl-N-(4-(4,4,5,5-tetramethyl-1,3,2-dioxaborolan-2-yl)benzyl)benzofuro[3,2-d]pyrimidin-4-amine (34.8 mg, 0.084 mmol). The solids were suspended in THF (5 mL). The mixture was treated with 0.5M K₃PO₄ (0.537 mL, 0.269 mmol) and X-Phos precatalyst G2 (3.96 mg, 5.03 μmol). Argon was streamed over the mixture for 5 minutes. The flask was capped and stirred at RT for 16 hours. LC/MS showed some desired product. After cooling to RT, removed solvent under a stream of air and took up residue in 3 mL of DCM and added 30 mg of Quadrasil AP to help remove active palladium remaining in the reaction mixture. After stirring 45 minutes under air, the mixture was filtered and concentrated down under a stream of air to give a residue. 10M sodium hydroxide (0.101 mL, 1.007 mmol) was added and the vial capped and heated to 80° C. for 16 hours. LC/MS showed that the hydrolysis was complete. The reaction mixture was cooled, then filtered and was purified via preparative LC/MS to give 4.1 mg (9%) of the desired compound. LCMS (M+1)=642.3. ¹H NMR (500 MHz, DMSO-d₆) δ 8.55 (s, 1H), 8.11 (d, J=7.7 Hz, 1H), 7.97 (s, 1H), 7.78-7.72 (m, 1H), 7.69 (t, J=7.7 Hz, 1H), 7.50 (t, J=7.5 Hz, 1H), 7.45 (d, J=7.7 Hz, 2H), 7.32 (d, J=8.1 Hz, 2H), 5.49 (s, 1H), 5.31-5.08 (m, 2H), 2.55 (s, 2H), 1.91 (s, 6H), 1.14 (s, 13H), 0.66 (br s, 6H).

### Example 221

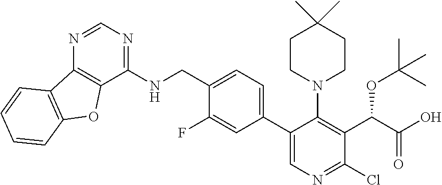 [SMILES: CC1(C)CCN(c2c(-c3ccc(CNc4ncnc5c4oc4ccccc45)c(F)c3)cnc(Cl)c2[C@H](OC(C)(C)C)C(=O)O)CC1]

### (S)-2-(5-(4-((Benzofuro[3, 2-d]pyrimidin-4-ylamino)methyl)-3-fluorophenyl)-2-chloro-4-(4, 4-dimethylpiperidin-1-yl)pyridin-3-yl)-2-(tert-butoxy)acetic acid

In a pressure vial equipped with a magnetic stirring bar was added (S)-ethyl 2-(5-bromo-2-chloro-4-(4,4-dimethylpiperidin-1-yl)pyridin-3-yl)-2-(tert-butoxy)acetate (60 mg, 0.130 mmol) and N-(2-fluoro-4-(4,4,5,5-tetramethyl-1,3,2-dioxaborolan-2-yl)benzyl)benzofuro[3,2-d]pyrimidin-4-amine (82 mg, 0.195 mmol). The solids were suspended in distilled THF (5 mL). The mixture was treated with 0.5M K₃PO₄ (0.909 mL, 0.455 mmol) and X-Phos precatalyst G2 (8.69 mg, 0.011 mmol). Argon was streamed over and bubbled into the mixture for 5 minutes with sonication. The vial was capped and stirred at RT for 16 hours. LC/MS showed the desired ester intermediate. Removed water layer and then removed solvent under vacuum. The solids were suspend in ethanol (8 mL) and transferred into a pressure vial. 10M sodium hydroxide (0.130 mL, 1.299 mmol) was added, and the vial capped and heated to 80° C. for 16 hours. LC/MS showed some of the hydrolysis product. The reaction mixture was cooled, then filtered and was purified via preparative LC/MS to give 5.6 mg (7%) of the desired compound. LCMS (M+1)=646.2.

¹H NMR (500 MHz, DMSO-d₆) δ 8.61-8.52 (m, 1H), 8.48 (s, 1H), 8.10 (d, J=7.7 Hz, 1H), 8.01 (s, 1H), 7.79 (d, J=8.4 Hz, 1H), 7.75-7.64 (m, 1H), 7.59-7.46 (m, 2H), 7.24 (br d, J=11.0 Hz, 1H), 7.12 (d, J=7.3 Hz, 1H), 5.55 (s, 1H), 4.93-4.84 (m, 2H), 2.55 (s, 2H), 1.34-1.11 (m, 16H), 0.78-0.64 (m, 6H).

### Example 222

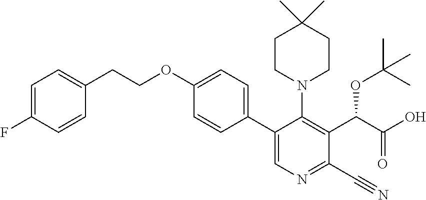 [SMILES: CC1(C)CCN(c2c(-c3ccc(OCCc4ccc(F)cc4)cc3)cnc(C#N)c2[C@H](OC(C)(C)C)C(=O)O)CC1]

### (S)-2-(tert-Butoxy)-2-(2-cyano-4-(4, 4-dimethylpiperidin-1-yl)-5-(4-(4-fluorophenethoxy)phenyl)pyridin-3-yl)acetic acid

In a microwave pressure vessel equipped with a magnetic stirring bar was added purified (S)-ethyl 2-(tert-butoxy)-2-(2-chloro-4-(4,4-dimethylpiperidin-1-yl)-5-(4-(4-fluorophenethoxy)phenyl)pyridin-3-yl)acetate (30 mg, 0.050 mmol), tetrakis(triphenylphosphine)palladium(0) (14.51 mg, 0.013 mmol) in degassed with argon, DMF (2 mL). Dicyanozinc (20.64 mg, 0.176 mmol) was then added and the vial was purged with argon, capped and heated to 160° C. within a microwave reactor for 2 hours. LC/MS showed the desired M+1 product after 2 h of heating at 160° C. within the microwave reactor as a minor product. The reaction mixture was purified by TFA buffer HPLC. Like fractions were dried down under a stream of nitrogen overnight. LC/MS showed a peak for the desired compound. The reaction mixture was purified via preparative LC/MS to give 2.5 mg (9%) of the desired compound. LCMS (M+1)=559.8. ¹H NMR (500 MHz, DMSO-d6) δ 8.19 (s, 1H), 7.38 (t, J=6.7 Hz, 2H), 7.25 (d, J=8.4 Hz, 2H), 7.20-7.10 (m, 2H), 7.07 (d, J=8.8 Hz, 2H), 5.31 (s, 1H), 4.33-4.16 (m, 2H), 3.06 (t, J=6.8 Hz, 1H), 1.88 (s, 3H), 1.43-1.30 (m, 1H), 1.30-1.19 (m, 2H), 1.11 (s, 9H), 0.83 (s, 6H), 0.44 (s, 1H), 0.37 (s, 2H), 0.30 (s, 1H).

### Example 223

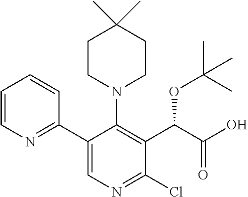 [SMILES: CC1(C)CCN(c2c(-c3ccccn3)cnc(Cl)c2[C@H](OC(C)(C)C)C(=O)O)CC1]

### (2S)-2-(tert-Butoxy)-2-[6′-chloro-4′-(4, 4-dimethylpiperidin-1-yl)-[2, 3′-bipyridine]-5′-yl]acetic acid

General method D was followed on a 54 μmol reaction scale using 6-methyl-2-(pyridin-2-yl)-1,3,6,2-dioxazaborocane-4,8-dione and (S)-ethyl 2-(5-bromo-2-chloro-4-(4,4-dimethylpiperidin-1-yl)pyridin-3-yl)-2-(tert-butoxy)acetate as starting material. The crude material was purified via preparative LC/MS to afford (2S)-2-(tert-butoxy)-2-[6′-chloro-4′-(4,4-dimethylpiperidin-1-yl)-[2,3′-bipyridine]-5′-yl]acetic acid (7.3 mg, 31%). LCMS (M+H)=432.11. ¹H NMR (500 MHz, methanol-d4) δ 8.66 (br d, J=4.6 Hz, 1H), 8.07 (s, 1H), 7.94 (td, J=7.7, 1.7 Hz, 1H), 7.54 (d, J=7.6 Hz, 1H), 7.47 (dd, J=7.6, 4.9 Hz, 1H), 5.74 (s, 1H), 3.00-2.69 (m, 4H), 1.30-1.28 (m, 4H), 1.23 (s, 9H), 0.83 (s, 6H).

### Example 224

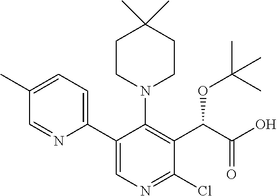 [SMILES: Cc1ccc(-c2cnc(Cl)c([C@H](OC(C)(C)C)C(=O)O)c2N2CCC(C)(C)CC2)nc1]

### (S)-2-(tert-Butoxy)-2-(6′-chloro-4′-(4, 4-dimethylpiperidin-1-yl)-5-methyl-[2, 3′-bipyridin]-5′-yl)acetic acid

General method D was followed on a 54 μmol reaction scale using 6-methyl-2-(5-methylpyridin-2-yl)-1,3,6,2-dioxazaborocane-4,8-dione and (S)-ethyl 2-(5-bromo-2-chloro-4-(4,4-dimethylpiperidin-1-yl)pyridin-3-yl)-2-(tert-butoxy)acetate as starting material. The crude material was purified via preparative LC/MS to afford (S)-2-(tert-butoxy)-2-(6′-chloro-4′-(4,4-dimethylpiperidin-1-yl)-5-methyl-[2,3′-bipyridin]-5′-yl)acetic acid (1.2 mg, 4%). LCMS (M+H)=446.1. ¹H NMR (500 MHz, methanol-d4) δ 8.54 (s, 1H), 8.11 (s, 1H), 7.86-7.78 (m, 1H), 7.46 (d, J=7.6 Hz, 1H), 5.82 (s, 1H), 2.46 (s, 3H), 1.46-1.34 (m, 4H), 1.28 (s, 9H), 0.85 (s, 6H).

### Example 225

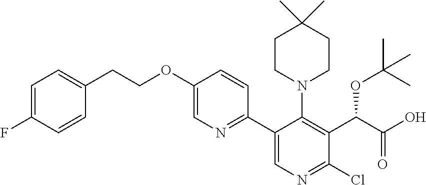 [SMILES: CC1(C)CCN(c2c(-c3ccc(OCCc4ccc(F)cc4)cn3)cnc(Cl)c2[C@H](OC(C)(C)C)C(=O)O)CC1]

### (2S)-2-(tert-Butoxy)-2-[6′-chloro-4′-(4, 4-dimethylpiperidin-1-yl)-5-[2-(4-fluorophenyl)ethoxy]-[2, 3′-bipyridine]-5′-yl]acetic acid

General method D was followed on a 54 μmol reaction scale using 2-(5-(4-fluorophenethoxy)pyridin-2-yl)-6-methyl-1,3,6,2-dioxazaborocane-4,8-dione and (S)-ethyl 2-(5-bromo-2-chloro-4-(4,4-dimethylpiperidin-1-yl)pyridin-3-yl)-2-(tert-butoxy)acetate as starting material. The crude material was purified via preparative LC/MS to afford (2S)-2-(tert-butoxy)-2-[6′-chloro-4′-(4,4-dimethylpiperidin-1-yl)-5-[2-(4-fluorophenyl)ethoxy]-[2,3′-bipyridine]-5′-yl]acetic acid (0.9 mg, 2%). LCMS (M+H)=570.3. ¹H NMR (500 MHz, methanol-d4) δ 8.35 (d, J=2.7 Hz, 1H), 8.10 (s, 1H), 7.53-7.49 (m, 1H), 7.47-7.43 (m, 1H), 7.37-7.32 (m, 2H), 7.04 (t, J=8.9 Hz, 2H), 5.81 (s, 1H), 4.36 (td, J=6.6, 1.8 Hz, 2H), 3.14 (t, J=6.6 Hz, 2H), 2.79 (br s, 4H), 1.37-1.32 (m, 4H), 1.27 (s, 9H), 0.85 (s, 6H).

### Example 226

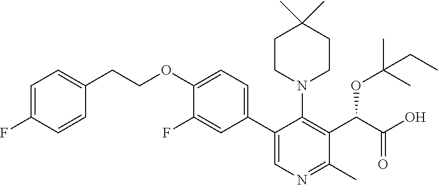 [SMILES: CCC(C)(C)O[C@H](C(=O)O)c1c(C)ncc(-c2ccc(OCCc3ccc(F)cc3)c(F)c2)c1N1CCC(C)(C)CC1]

### (S)-2-(4-(4, 4-Dimethylpiperidin-1-yl)-5-(3-fluoro-4-(4-fluorophenethoxy)phenyl)-2-methylpyridin-3-yl)-2-(tert-pentyloxy)acetic acid

To a 14 mL test tube equipped with a stir was added tribasic potassium phosphate (407 mg, 1.917 mmol), 2-(3-fluoro-4-(4-fluorophenethoxy)phenyl)-4,4,5,5-tetramethyl-1,3,2-dioxaborolane (92 mg, 0.256 mmol), isopropyl (S)-2-(5-bromo-4-(4,4-dimethylpiperidin-1-yl)-2-methylpyridin-3-yl)-2-(tert-pentyloxy)acetate (100 mg, 0.213 mmol), and SPhos-Pd-G3 (8.30 mg, 10.65 μmol). The test tube was sealed with a rubber septum and then placed under N₂ atm (vac/fill×3). To the flask was added dioxane (1.5 mL)+water (0.5 mL) (degassed for 5 minutes via N₂ bubbling). The test tube was placed in a 60° C. heating block with stirring for 18 h. The reaction solution was diluted with Et₂O (5 mL), then washed with water (5 mL). The organic phase was dried over MgSO₄; filtered into a 7 mL vial; then concentrated under a N₂ stream. To the vial was added a stir bar and Ethanol (4 mL), then sodium hydroxide (0.4 mL, 2.000 mmol). The vial was capped, then placed in a 80° C. hot plate with stirring for t=20 h. The reaction solution was filtered; and the filtrate was purified via HPLC purification on the InterChim system. The first pass was in water:MeCN w/0.1% TFA modified. The second pass was in water:MeCN w 10 mM NH₄OAc. Fractions were selected based on mass purity, and purity as measured by LCMS. The combined samples were concentrated in vacuo and the aqueous solution was frozen with dry ice, then lyophilized. The experiment afforded the desired product (S)-2-(4-(4,4-dimethylpiperidin-1-yl)-5-(3-fluoro-4-(4-fluorophenethoxy)phenyl)-2-methylpyridin-3-yl)-2-(tert-pentyloxy)acetic acid (36.0 mg, 0.061 mmol, 28% yield). ¹H NMR (500 MHz, methanol-d4) δ 8.05 (s, 1H), 7.35 (dd, J=8.5, 5.5 Hz, 2H), 7.19 (t, J=8.5 Hz, 1H), 7.11 (dd, J=11.7, 2.0 Hz, 1H), 7.07-6.99 (m, 3H), 5.85 (s, 1H), 4.34 (t, J=6.7 Hz, 2H), 3.13 (t, J=6.6 Hz, 2H), 3.10-3.00 (m, 2H), 2.82-2.69 (m, 2H), 2.65 (s, 3H), 1.64-1.47 (m, 2H), 1.46-1.37 (m, 4H), 1.19 (s, 3H), 1.13 (s, 3H), 0.89 (s, 6H), 0.79 (t, J=7.5 Hz, 3H). ESI-MS(+) m/z=581.3 (M+1).

### Example 227

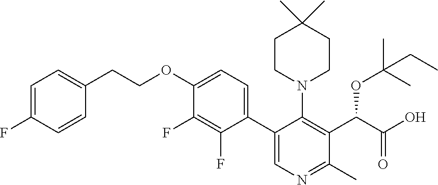 [SMILES: CCC(C)(C)O[C@H](C(=O)O)c1c(C)ncc(-c2ccc(OCCc3ccc(F)cc3)c(F)c2F)c1N1CCC(C)(C)CC1]

### (S)-2-(5-(2, 3-Difluoro-4-(4-fluorophenethoxy)phenyl)-4-(4, 4-dimethylpiperidin-1-yl)-2-methylpyridin-3-yl)-2-(tert-pentyloxy)acetic acid

To a 14 mL test tube equipped with a stir was added tribasic potassium phosphate (407 mg, 1.917 mmol), 2-(2,3-difluoro-4-(4-fluorophenethoxy)phenyl)-6-methyl-1,3,6,2-dioxazaborocane-4,8-dione (104 mg, 0.256 mmol), isopropyl (S)-2-(5-bromo-4-(4,4-dimethylpiperidin-1-yl)-2-methylpyridin-3-yl)-2-(tert-pentyloxy)acetate (100 mg, 0.213 mmol), and SPhos-Pd-G3 (8.30 mg, 10.65 μmol). The test tube was sealed with a rubber septum and then placed under N₂ atm (vac/fill×3). To the flask was added dioxane (1.5 mL)+water (0.5 mL) (degassed for 5 minutes via N₂ bubbling). The test tube was placed in a 60° C. heating block with stirring for t=18 hrs. The reaction solution was diluted with Et₂O (5 mL), then washed with water (5 mL). The organic phase was dried over MgSO₄; filtered into a 7 mL vial; then concentrated under a N₂ stream. To the vial was added a stir bar and Ethanol (4 mL), then aq. 5 N sodium hydroxide (0.15 mL, 0.750 mmol). The vial was capped, then placed in a 80° C. heating block with stirring. LCMS analysis at 20 h found a major peak corresponding to the desired product. The reaction solution filtered; the filtrate was subjected to HPLC purification on the InterChim system with ESI detection. The eluent was water:MeCN with 0.1% TFA as modifier. The product fractions were identified by mass, then concentrated overnight in the Genevac. The resulting residue was dissolved in MeOH and then subjected to a second round of HPLC purification on the InterChim system. The eluent was water:MeCN w/10 mM NH₄OAc as modifier. The product fractions were selected based on LCMS analysis. The combined sample was frozen, then lyophilized to afford a low density white solid. (S)-2-(5-(2,3-difluoro-4-(4-fluorophenethoxy)phenyl)-4-(4,4-dimethylpiperidin-1-yl)-2-methylpyridin-3-yl)-2-(tert-pentyloxy)acetic acid (12.3 mg, 0.021 mmol, 9.64% yield). ESI-MS(+) m/z (TFA buffer)=599.2 (M+1) retention time=1.33 min. ESI-MS(+) m/z (AA buffer)=599.3 (M+1) retention time=2.31 min.

### Example 228

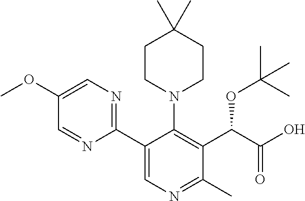 [SMILES: COc1cnc(-c2cnc(C)c([C@H](OC(C)(C)C)C(=O)O)c2N2CCC(C)(C)CC2)nc1]

### (S)-2-(tert-Butoxy)-2-(4-(4, 4-dimethylpiperidin-1-yl)-5-(5-methoxypyrimidin-2-yl)-2-methylpyridin-3-yl)acetic acid

To a solution of isopropyl (S)-2-(tert-butoxy)-2-(4-(4,4-dimethylpiperidin-1-yl)-5-(5-methoxypyrimidin-2-yl)-2-methylpyridin-3-yl)acetate (26 mg, 0.054 mmol) in EtOH (1 mL) and Water (0.100 mL) was added lithium hydroxide monohydrate (22.51 mg, 0.536 mmol) and heated at 75° C. for 24 hrs. The reaction was cooled to RT, filtered through a nylon 0.45μ frit filter and purified via preparative LC/MS. Fractions containing the desired product were combined and dried via centrifugal evaporation to afford the product (S)-2-(tert-butoxy)-2-(4-(4,4-dimethylpiperidin-1-yl)-5-(5-methoxypyrimidin-2-yl)-2-methylpyridin-3-yl)acetic acid (12.8 mg, 0.029 mmol, 53.9% yield). ¹H NMR (500 MHz, methanol-d4) δ 8.65 (s, 2H), 8.32 (s, 1H), 5.98 (s, 1H), 4.04 (s, 3H), 3.04-2.79 (m, 4H), 2.65 (s, 3H), 1.45 (br s, 4H), 1.22 (s, 9H), 0.93 (s, 6H). ESI-MS(+) m/z=443.2 (M+1).

### Example 229

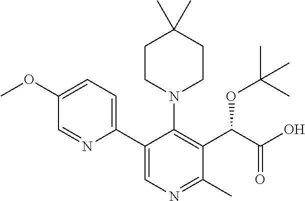 [SMILES: COc1ccc(-c2cnc(C)c([C@H](OC(C)(C)C)C(=O)O)c2N2CCC(C)(C)CC2)nc1]

### (S)-2-(tert-Butoxy)-2-(4′-(4, 4-dimethylpiperidin-1-yl)-5-methoxy-6′-methyl-[2, 3′-bipyridin]-5′-yl)acetic acid

To a solution of isopropyl (S)-2-(tert-butoxy)-2-(4′-(4,4-dimethylpiperidin-1-yl)-5-methoxy-6′-methyl-[2,3′-bipyridin]-5′-yl)acetate (22 mg, 0.045 mmol) in EtOH (1 mL) and water (0.100 mL) was added lithium hydroxide monohydrate (19.09 mg, 0.455 mmol) and heated at 75° C. for 24 hrs. The reaction was cooled to RT, filtered through a nylon 0.45μ frit filter and purified via preparative LC/MS. Fractions containing the desired product were combined and dried via centrifugal evaporation to afford the product (S)-2-(tert-butoxy)-2-(4′-(4,4-dimethylpiperidin-1-yl)-5-methoxy-6′-methyl-[2,3′-bipyridin]-5′-yl)acetic acid (13.4 mg, 0.030 mmol, 66.7% yield). ¹H NMR (500 MHz, methanol-d4) δ 8.37 (d, J=2.9 Hz, 1H), 8.14 (s, 1H), 7.54 (dd, J=8.6, 2.8 Hz, 1H), 7.45 (d, J=8.8 Hz, 1H), 5.91 (s, 1H), 3.96 (s, 3H), 3.06-2.75 (m, 4H), 2.65 (s, 3H), 1.47-1.37 (m, 4H), 1.21 (s, 9H), 0.89 (s, 6H). ESI-MS(+) m/z=442.3 (M+1).

### Example 230

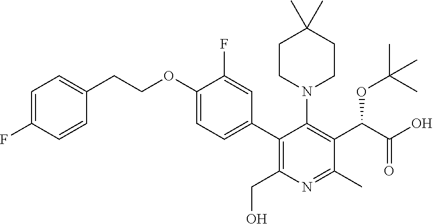 [SMILES: Cc1nc(CO)c(-c2ccc(OCCc3ccc(F)cc3)c(F)c2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(3-fluoro-4-(4-fluorophenethoxy)phenyl)-6-(hydroxymethyl)-2-methylpyridin-3-yl)acetic acid

To a 20 mL pressure vial under N₂ was added isopropyl (S)-2-(tert-butoxy)-2-(4-(4,4-dimethylpiperidin-1-yl)-5-(3-fluoro-4-(4-fluorophenethoxy)phenyl)-6-(hydroxymethyl)-2-methylpyridin-3-yl)acetate (62.7 mg, 0.098 mmol), ethanol (4.5 mL) and 10 M sodium hydroxide (140 μl, 1.400 mmol). The reaction was flushed briefly with N₂, capped and heated at 105° C. for 7.5 h, followed by room temp for 18 h. The reaction was treated with MeOH (100 μlit) and additional 10 M NaOH (15 lit, 150 mmol) and heated at 105° C. for 8 h. The crude material was purified via preparative LCMS to afford (S)-2-(tert-butoxy)-2-(4-(4,4-dimethylpiperidin-1-yl)-5-(3-fluoro-4-(4-fluorophenethoxy)phenyl)-6-(hydroxymethyl)-2-methylpyridin-3-yl)acetic acid, 37.3 mg (64%). LCMS=597.3 (M+H).

### Example 231

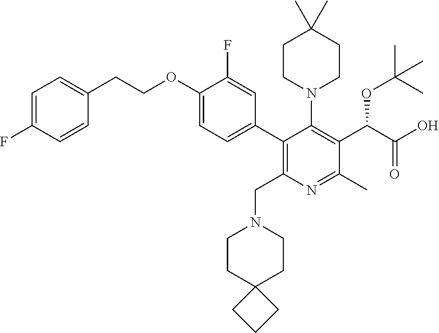 [SMILES: Cc1nc(CN2CCC3(CCC3)CC2)c(-c2ccc(OCCc3ccc(F)cc3)c(F)c2)c(N2CCC(C)(C)CC2)c1[C@H](OC(C)(C)C)C(=O)O]

### (S)-2-(6-((7-Azaspiro[3.5]nonan-7-yl)methyl)-4-(4, 4-dimethylpiperidin-1-yl)-5-(3-fluoro-4-(4-fluorophenethoxy)phenyl)-2-methylpyridin-3-yl)-2-(tert-butoxy)acetic acid

To a dry reaction vial under nitrogen was added isopropyl (S)-2-(tert-butoxy)-2-(4-(4,4-dimethylpiperidin-1-yl)-5-(3-fluoro-4-(4-fluorophenethoxy)phenyl)-6-formyl-2-methylpyridin-3-yl)acetate (50 mg, 0.079 mmol), 7-azaspiro[3.5]nonane (37.2 mg, 0.297 mmol), ClCH₂CH₂Cl (2.0 mL), acetic acid (18 μL, 0.314 mmol) and several pieces of 4 A° molecular sieves. The reaction was stirred at room temp for 10 min, then treated with EtOH (1.0 mL) (previously dried over 4 A° molecular sieves). The reaction was stirred at room temp for 25 min, then treated (very slowly, dropwise, over several hours) with sodium cyanoborohydride, 1.0 M in THF (300 μL, 0.300 mmol). After the addition was complete, the solvent was evaporated under a gentle stream of N2. The resulting residue was redissolved in a mixture of ethanol (4 mL) and methanol (400 μlit), treated with 10 M sodium hydroxidein (115 μL, 1.150 mmol) and heated at 105° C. for 20 h. The crude material was purified via preparative LCMS to afford (S)-2-(6-((7-azaspiro[3.5]nonan-7-yl)methyl)-4-(4,4-dimethylpiperidin-1-yl)-5-(3-fluoro-4-(4-fluorophenethoxy)phenyl)-2-methylpyridin-3-yl)-2-(tert-butoxy)acetic acid, 31.6 mg (56%). LCMS=704.3 (M+H).

### Example 232

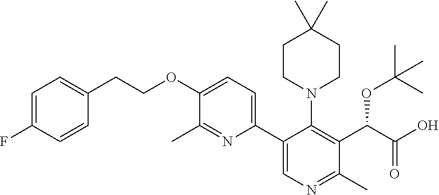 [SMILES: Cc1nc(-c2cnc(C)c([C@H](OC(C)(C)C)C(=O)O)c2N2CCC(C)(C)CC2)ccc1OCCc1ccc(F)cc1]

### (S)-2-(tert-Butoxy)-2-(4′-(4, 4-dimethylpiperidin-1-yl)-5-(4-fluorophenethoxy)-6, 6′-dimethyl-[2, 3′-bipyridin]-5′-yl)acetic acid

To a solution of isopropyl (S)-2-(tert-butoxy)-2-(4′-(4,4-dimethylpiperidin-1-yl)-5-(4-fluorophenethoxy)-6,6′-dimethyl-[2,3′-bipyridin]-5′-yl)acetate (20 mg, 0.033 mmol) in EtOH (1 mL) and Water (0.100 mL) was added lithium hydroxide monohydrate (13.85 mg, 0.330 mmol) and heated at 75° C. for 24 hrs. The reaction was cooled to RT, filtered through a nylon 0.45μ frit filter and purified via preparative LCMS. Fractions containing the desired product were combined and dried via centrifugal evaporation to afford the product (S)-2-(tert-butoxy)-2-(4′-(4,4-dimethylpiperidin-1-yl)-5-(4-fluorophenethoxy)-6,6′-dimethyl-[2,3′-bipyridin]-5′-yl)acetic acid (6.9 mg, 0.012 mmol, 36.7% yield). ¹H NMR (500 MHz, methanol-d4) δ 8.12 (s, 1H), 7.43 (d, J=8.4 Hz, 1H), 7.38-7.31 (m, 2H), 7.27 (d, J=8.4 Hz, 1H), 7.04 (t, J=8.8 Hz, 2H), 5.90 (s, 1H), 4.33 (t, J=6.4 Hz, 2H), 3.16 (t, J=6.4 Hz, 2H), 3.02-2.72 (m, 4H), 2.64 (s, 3H), 2.43 (s, 3H), 1.46-1.37 (m, 4H), 1.21 (s, 9H), 0.88 (s, 6H). ESI-MS(+) m/z=564.2 (M+1).

### Example 233

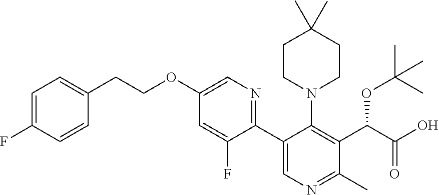 [SMILES: Cc1ncc(-c2ncc(OCCc3ccc(F)cc3)cc2F)c(N2CCC(C)(C)CC2)c1[C@H](OC(C)(C)C)C(=O)O]

### (S)-2-(tert-Butoxy)-2-(4′-(4, 4-dimethylpiperidin-1-yl)-3-fluoro-5-(4-fluorophenethoxy)-6′-methyl-[2, 3′-bipyridin]-5′-yl)acetic acid

To a solution of isopropyl (S)-2-(tert-butoxy)-2-(4′-(4,4-dimethylpiperidin-1-yl)-3-fluoro-5-(4-fluorophenethoxy)-6′-methyl-[2,3′-bipyridin]-5′-yl)acetate (24 mg, 0.039 mmol) in EtOH (1 mL) and water (0.100 mL) was added lithium hydroxide monohydrate (16.52 mg, 0.394 mmol) and heated at 75° C. for 24 hrs. The reaction was cooled to RT, filtered through a nylon 0.45 g frit filter and purified via preparative LC/MS. Fractions containing the desired product were combined and dried via centrifugal evaporation to afford the product (S)-2-(tert-butoxy)-2-(4′-(4,4-dimethylpiperidin-1-yl)-3-fluoro-5-(4-fluorophenethoxy)-6′-methyl-[2,3′-bipyridin]-5′-yl)acetic acid (10.9 mg, 0.019 mmol, 48.3% yield). ¹H NMR (500 MHz, methanol-d4) δ 8.22 (d, J=2.2 Hz, 1H), 8.02 (s, 1H), 7.39-7.32 (m, 3H), 7.04 (t, J=8.8 Hz, 2H), 5.80 (s, 1H), 4.40-4.30 (m, 2H), 3.14 (t, J=6.4 Hz, 2H), 3.04-2.72 (m, 4H), 2.64 (s, 3H), 1.40 (br s, 4H), 1.19 (s, 9H), 0.87 (s, 6H). ESI-MS(+) m/z=568.9 (M+1).

### Example 234

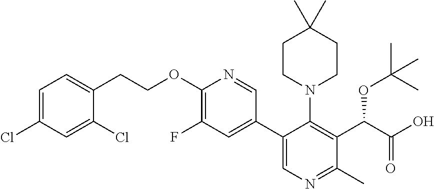 [SMILES: Cc1ncc(-c2cnc(OCCc3ccc(Cl)cc3Cl)c(F)c2)c(N2CCC(C)(C)CC2)c1[C@H](OC(C)(C)C)C(=O)O]

### (S)-2-(tert-Butoxy)-2-(6′-(2, 4-dichlorophenethoxy)-4-(4, 4-dimethylpiperidin-1-yl)-5′-fluoro-6-methyl-[3, 3′-bipyridin]-5-yl)acetic acid

A mixture of 2-(2,4-dichlorophenyl)ethan-1-ol (0.045 g, 0.235 mmol, 5 equiv), 60% NaH (9.39 mg, 0.235 mmol, 5 equiv), and isopropyl (S)-2-(tert-butoxy)-2-(4-(4,4-dimethylpiperidin-1-yl)-5′,6′-difluoro-6-methyl-[3,3′-bipyridin]-5-yl)acetate (0.023 g, 0.047 mmol, 1 equiv) in THF (1.5 mL) was stirred for 1 h. Upon complete addition, 5 NaOH (0.167 mL, 0.837 mmol, 10 equiv) was added and the mixture was heated at 80° C. for 2 h. After cooling to ambient temperature, the mixture was filtered and purified by reverse phase preparative HPLC to give the product (12.4 mg, 42%). ¹H NMR (500 MHz, DMSO-d₆) δ 8.10-8.06 (m, 1H), 7.92 (d, J=1.8 Hz, 1H), 7.73-7.67 (m, 1H), 7.57 (d, J=2.2 Hz, 1H), 7.47 (d, J=8.4 Hz, 1H), 7.39 (dd, J=8.1, 2.2 Hz, 1H), 5.81 (s, 1H), 4.73-4.65 (m, 2H), 2.52 (s, 2H), 1.36-1.28 (m, 3H), 1.14 (s, 10H), 0.88-0.78 (m, 6H). [note: some piperidine protons not seen]. LCMS (M+1): 618.2.

### Example 235

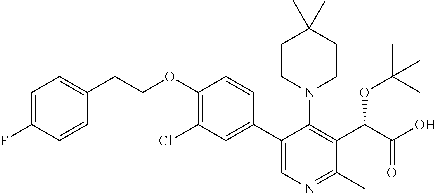 [SMILES: Cc1ncc(-c2ccc(OCCc3ccc(F)cc3)c(Cl)c2)c(N2CCC(C)(C)CC2)c1[C@H](OC(C)(C)C)C(=O)O]

### (S)-2-(tert-Butoxy)-2-(5-(3-chloro-4-(4-fluorophenethoxy)phenyl)-4-(4, 4-dimethylpiperidin-1-yl)-2-methylpyridin-3-yl)acetic acid

A solution of isopropyl (S)-2-(5-bromo-4-(4,4-dimethylpiperidin-1-yl)-2-methylpyridin-3-yl)-2-(tert-butoxy)acetate (0.10 g, 0.220 mmol, 1 equiv), 2-(3-chloro-4-(4-fluorophenethoxy)phenyl)-4,4,5,5-tetramethyl-1,3,2-dioxaborolane (0.124 g, 0.329 mmol, 1.5 equiv), Pd(dppf)Cl₂ (16 mg, 0.022 mmol, 0.1 equiv), and 2 M K₃PO₄ (0.66 ml, 1.32 mmol, 6 equiv) in dioxane (2.2 mL) was heated at 80° C. for 2 h. After cooling to ambient temperature, the reaction mixture was diluted with ethyl acetate and washed with brine. The organic layer was dried (Na₂SO₄) and concentrated in vacuo. The residue was taken up in ethanol (2 mL) and 10 M NaOH (0.2 mL) was added. The mixture was heated at 80° C. for 4 h. After cooling to ambient temperature, the mixture was filtered and purified by reverse phase preparative HPLC to provide the product (40 mg, 32%). ¹H NMR (500 MHz, DMSO-d₆) δ 8.05 (s, 1H), 7.41 (dd, J=8.4, 5.5 Hz, 2H), 7.36 (d, J=1.8 Hz, 1H), 7.27-7.21 (m, 2H), 7.16-7.10 (m, 2H), 5.83 (s, 1H), 4.37-4.29 (m, 2H), 3.10 (t, J=6.4 Hz, 2H), 2.50 (s, 3H), 1.65-1.38 (m, 1H), 1.32 (br d, J=2.2 Hz, 3H), 1.14 (s, 9H), 0.83 (br s, 6H) [note: some piperidine protons not visible]. LCMS (M+1): 583.2.

### Example 236

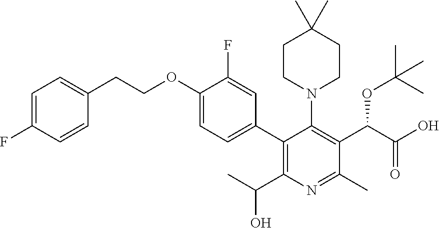 [SMILES: Cc1nc(C(C)O)c(-c2ccc(OCCc3ccc(F)cc3)c(F)c2)c(N2CCC(C)(C)CC2)c1[C@H](OC(C)(C)C)C(=O)O]

### (2S)-2-(tert-Butoxy)-2-(4-(4, 4-dimethylpiperidin-1-yl)-5-(3-fluoro-4-(4-fluorophenethoxy)phenyl)-6-(1-hydroxyethyl)-2-methylpyridin-3-yl)acetic acid

To a dry reaction vial under N₂ was added isopropyl (2S)-2-(tert-butoxy)-2-(4-(4,4-dimethylpiperidin-1-yl)-5-(3-fluoro-4-(4-fluorophenethoxy)phenyl)-6-(1-hydroxyethyl)-2-methylpyridin-3-yl)acetate (15.3 mg, 0.023 mmol, diastereomer 1), EtOH (3 mL), methanol (500 μL) and 10 M sodium hydroxide (35 μL, 0.350 mmol). The reaction is flushed briefly with N2, capped and heated at 105° C. for 6 h. The reaction was treated with additional sodium hydroxide, 10M in water (10 mlit, 0.100 mmol) and heated at 105° C. for 18 h. The crude material was purified via preparative LCMS to afford (2S)-2-(tert-butoxy)-2-(4-(4,4-dimethylpiperidin-1-yl)-5-(3-fluoro-4-(4-fluorophenethoxy)phenyl)-6-(1-hydroxyethyl)-2-methylpyridin-3-yl)acetic acid (diastereomer 1), 13.4 mg (82%). LCMS=611.3 (M+H).

### Example 237

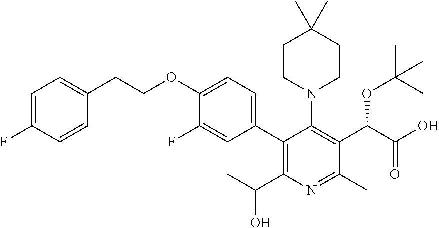 [SMILES: Cc1nc(C(C)O)c(-c2ccc(OCCc3ccc(F)cc3)c(F)c2)c(N2CCC(C)(C)CC2)c1[C@H](OC(C)(C)C)C(=O)O]

### (2S)-2-(tert-Butoxy)-2-(4-(4, 4-dimethylpiperidin-1-yl)-5-(3-fluoro-4-(4-fluorophenethoxy)phenyl)-6-(1-hydroxyethyl)-2-methylpyridin-3-yl)acetic acid

To a dry reaction vial under N₂ was added isopropyl (2S)-2-(tert-butoxy)-2-(4-(4,4-dimethylpiperidin-1-yl)-5-(3-fluoro-4-(4-fluorophenethoxy)phenyl)-6-(1-hydroxyethyl)-2-methylpyridin-3-yl)acetate (9.7 mg, 0.015 mmol, diastereomer 2), EtOH (3 mL), methanol (500 μL) and 10 M sodium hydroxide (23 μL, 0.230 mmol). The reaction is flushed briefly with N2, capped and heated at 105° C. for 6 h. The reaction was treated with additional 10 M sodium hydroxide (23 μlit, 0.230 mmol) and heated at 105° C. for 18 h. The crude material was purified via preparative LCMS to afford (2S)-2-(tert-butoxy)-2-(4-(4,4-dimethylpiperidin-1-yl)-5-(3-fluoro-4-(4-fluorophenethoxy)phenyl)-6-(1-hydroxyethyl)-2-methylpyridin-3-yl)acetic acid (diastereomer 2), 13.4 mg (82%). LCMS=611.3 (M+H).

### Example 238

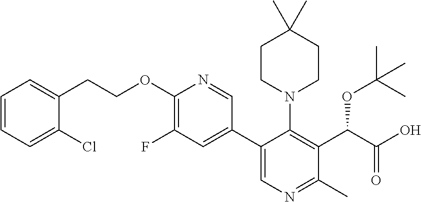 [SMILES: Cc1ncc(-c2cnc(OCCc3ccccc3Cl)c(F)c2)c(N2CCC(C)(C)CC2)c1[C@H](OC(C)(C)C)C(=O)O]

### (S)-2-(tert-Butoxy)-2-(6′-(2-chlorophenethoxy)-4-(4, 4-dimethylpiperidin-1-yl)-5′-fluoro-6-methyl-[3, 3′-bipyridin]-5-yl)acetic acid

A mixture of 2-(2-chlorophenyl)ethan-1-ol (0.037 g, 0.235 mmol, 5 equiv), 60% NaH (9.39 mg, 0.235 mmol, 5 equiv) and isopropyl (S)-2-(tert-butoxy)-2-(4-(4,4-dimethylpiperidin-1-yl)-5′,6′-difluoro-6-methyl-[3,3′-bipyridin]-5-yl)acetate (0.023 g, 0.047 mmol, 1 equiv) in THF (1.5 mL) was stirred for 1 h. Upon completion, 5 N NaOH (0.167 mL, 0.837 mmol, 10 equiv) was added and the mixture was heated at 80° C. for 2 h. After cooling to ambient temperature, the mixture was filtered and purified by reverse phase preparative HPLC to deliver the product (11.1 mg, 40%). ¹H NMR (500 MHz, DMSO-d₆) δ 8.12-8.03 (m, 1H), 7.98-7.89 (m, 1H), 7.73-7.64 (m, 1H), 7.49-7.38 (m, 2H), 7.34-7.23 (m, 2H), 5.77 (br d, J=2.6 Hz, 1H), 4.76-4.64 (m, 2H), 2.52 (s, 2H), 1.38-1.27 (m, 3H), 1.21-1.05 (m, 10H), 0.96-0.75 (m, 6H) [note: some piperidine protons not seen]. LCMS (M+1): 584.2.

### Example 239

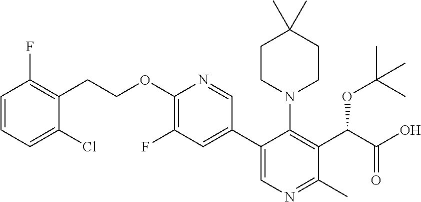 [SMILES: Cc1ncc(-c2cnc(OCCc3c(F)cccc3Cl)c(F)c2)c(N2CCC(C)(C)CC2)c1[C@H](OC(C)(C)C)C(=O)O]

### (S)-2-(tert-Butoxy)-2-(6′-(2-chloro-6-fluorophenethoxy)-4-(4, 4-dimethylpiperidin-1-yl)-5′-fluoro-6-methyl-[3, 3′-bipyridin]-5-yl)acetic acid

A mixture of 2-(2-chloro-6-fluorophenyl)ethan-1-ol (0.041 g, 0.235 mmol, 5 equiv), 60% NaH (9.39 mg, 0.235 mmol, 5 equiv) and isopropyl (S)-2-(tert-butoxy)-2-(4-(4,4-dimethylpiperidin-1-yl)-5′,6′-difluoro-6-methyl-[3,3′-bipyridin]-5-yl)acetate (0.023 g, 0.047 mmol, 1 equiv) in THF (1.5 mL) was stirred for 1 h. After complete addition, 5 N NaOH (0.167 mL, 0.837 mmol, 10 equiv) was added and the mixture was heated at 80° C. for 2 h. Upon cooling to ambient temperature, the mixture was filtered and purified by reverse phase preparative HPLC to provide the product (15 mg, 53%). ¹H NMR (500 MHz, DMSO-d₆) δ 8.09-8.01 (m, 1H), 7.95-7.88 (m, 1H), 7.68 (dd, J=11.0, 1.8 Hz, 1H), 7.38-7.28 (m, 2H), 7.25-7.17 (m, 1H), 5.79 (s, 1H), 4.77-4.64 (m, 2H), 1.41-1.28 (m, 3H), 1.14 (s, 10H), 0.91-0.76 (m, 6H) [note: some piperidine protons not seen]. LCMS (M+1): 602.2.

### Example 240

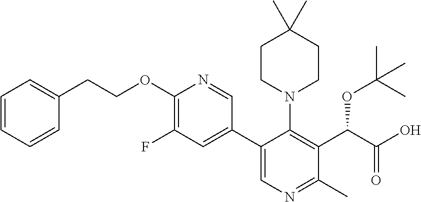 [SMILES: Cc1ncc(-c2cnc(OCCc3ccccc3)c(F)c2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5′-fluoro-6-methyl-6′-phenethoxy-[3,3′-bipyridin]-5-yl)acetic acid

A mixture of 2-phenylethan-1-ol (0.029 g, 0.235 mmol, 5 equiv), 60% NaH (9.39 mg, 0.235 mmol, 5 equiv), and isopropyl (S)-2-(tert-butoxy)-2-(4-(4,4-dimethylpiperidin-1-yl)-5′,6′-difluoro-6-methyl-[3,3′-bipyridin]-5-yl)acetate (0.023 g, 0.047 mmol, 1 equiv) in THF (1.5 mL) was stirred at ambient temperature for 1 h. Upon complete addition, 5 N NaOH (0.167 mL, 0.837 mmol, 10 equiv) was added and the mixture was heated at 80° C. for 2 h. After cooling to ambient temperature, the mixture was filtered and then purified by reverse phase preparative HPLC to give the product (18 mg, 71%). ¹H NMR (500 MHz, DMSO-d₆) δ 8.14-8.06 (m, 1H), 7.98-7.89 (m, 1H), 7.70 (dd, J=11.2, 1.7 Hz, 1H), 7.35-7.16 (m, 5H), 5.82 (s, 1H), 4.72-4.58 (m, 2H), 3.14-3.06 (m, 1H), 2.52 (s, 2H), 1.39-1.27 (m, 3H), 1.19-1.09 (m, 10H), 0.94-0.78 (m, 6H) [note: some piperidine protons not observed]. LCMS (M+1): 550.3.

### Example 241

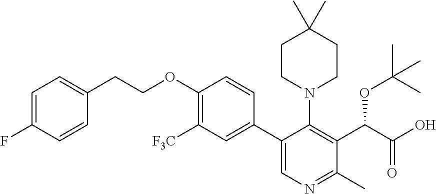 [SMILES: Cc1ncc(-c2ccc(OCCc3ccc(F)cc3)c(C(F)(F)F)c2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(4-fluorophenethoxy)-3-(trifluoromethyl)phenyl)-2-methylpyridin-3-yl)acetic acid

A mixture of isopropyl isopropyl (S)-2-(5-bromo-4-(4,4-dimethylpiperidin-1-yl)-2-methylpyridin-3-yl)-2-(tert-butoxy)acetate (0.04 g, 0.088 mmol, 1 equiv), 2-(4-(4-fluorophenethoxy)-3-(trifluoromethyl)phenyl)-4,4,5,5-tetramethyl-1,3,2-dioxaborolane (0.054 g, 0.132 mmol, 1.5 equiv), SPhos (7.21 mg, 0.018 mmol, 0.2 equiv), palladium(II) acetate (1.972 mg, 8.78 μmol, 0.1 equiv), and 2 M K₃PO₄ (0.132 ml, 0.263 mmol, 3 equiv) in dioxane was heated at 90° C. for 4 h. The reaction mixture was filtered through celite/Na₂SO₄ eluting with ethyl acetate and concentrated in vacuo. The residue was taken up in ethanol (1 mL) and 5 M NaOH (0.176 ml, 0.878 mmol, 10 equiv) was added. The mixture was heated at 90° C. for 4 h. After cooling to ambient temperature, the mixture was filtered and purified by reverse phase preparative HPLC to give the product (8 mg, 14%). ¹H NMR (500 MHz, DMSO-d₆) δ 8.09-8.03 (m, 1H), 7.57 (br d, J=8.8 Hz, 1H), 7.47 (d, J=1.8 Hz, 1H), 7.42-7.33 (m, 3H), 7.16-7.07 (m, 2H), 5.80 (s, 1H), 4.45-4.33 (m, 2H), 3.09 (t, J=6.4 Hz, 1H), 1.35-1.26 (m, 3H), 1.19-1.09 (m, 10H), 0.92-0.73 (m, 6H) [note: piperidine protons not all observed]. LCMS (M+1): 617.3.

### Example 242

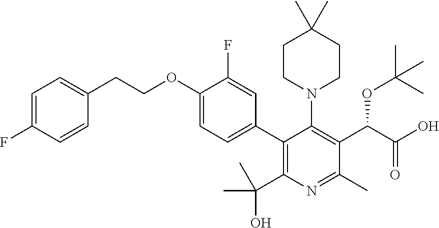 [SMILES: Cc1nc(C(C)(C)O)c(-c2ccc(OCCc3ccc(F)cc3)c(F)c2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(3-fluoro-4-(4-fluorophenethoxy)phenyl)-6-(2-hydroxypropan-2-yl)-2-methylpyridin-3-yl)acetic acid (Locked Atrop Isomer 1)

To a dry reaction vial under N₂ was added isopropyl (S)-2-(6-acetyl-4-(4,4-dimethylpiperidin-1l-yl)-5-(3-fluoro-4-(4-fluorophenethoxy)phenyl)-2-methylpyridin-3-yl)-2-(tert-butoxy)acetate (16.5 mg, 0.025 mmol) and THF (700 μL). The reaction was flushed very well with argon, then cooled to 0° C. in ice/water bath. The reaction was then treated with 3 M methylmagnesium chloride in THF (13 μL, 0.039 mmol) over 30 sec. The reaction was allowed to stir at 0° C. while slowly warming to room temp over 40 min. The reaction was quenched by the addition of ethanol (1.0 mL) and the solvent was removed under a gentle stream of N2. The residue was redissolved in ethanol (4 mL), treated with 10 M sodium hydroxide (45 μL, 0.450 mmol) and heated at 105° C. for 6 h. The reaction was treated with methanol (500 lit), additional 10 M sodium hydroxide (25 μlit, 0.250 mmol) and heated at 105° C. for 10.5 h. The crude material was purified via preparative LCMS to afford (S)-2-(tert-butoxy)-2-(4-(4,4-dimethylpiperidin-1-yl)-5-(3-fluoro-4-(4-fluorophenethoxy)phenyl)-6-(2-hydroxypropan-2-yl)-2-methylpyridin-3-yl)acetic acid (Atropisomer 1), 3.9 mg (9%). LCMS=625.3 (M+H).

Also isolated from this reaction was Atropisomer 2

### Example 243

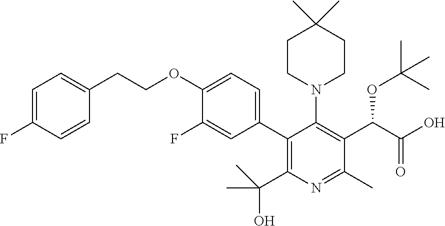 [SMILES: Cc1nc(C(C)(C)O)c(-c2ccc(OCCc3ccc(F)cc3)c(F)c2)c(N2CCC(C)(C)CC2)c1[C@H](OC(C)(C)C)C(=O)O]

(S)-2-(tert-butoxy)-2-(4-(4,4-dimethylpiperidin-1-yl)-5-(3-fluoro-4-(4-fluorophenethoxy)phenyl)-6-(2-hydroxypropan-2-yl)-2-methylpyridin-3-yl)acetic acid, 6.1 mg (24%). LCMS=625.3 (M+H).

### Example 244

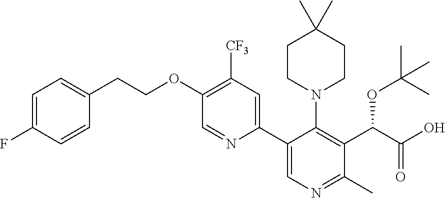 [SMILES: Cc1ncc(-c2cc(C(F)(F)F)c(OCCc3ccc(F)cc3)cn2)c(N2CCC(C)(C)CC2)c1[C@H](OC(C)(C)C)C(=O)O]

### (S)-2-(tert-Butoxy)-2-(4′-(4, 4-dimethylpiperidin-1-yl)-5-(4-fluorophenethoxy)-6′-methyl-4-(trifluoromethyl)-[2, 3′-bipyridin]-5′-yl)acetic acid

To a solution of isopropyl (S)-2-(tert-butoxy)-2-(4′-(4,4-dimethylpiperidin-1-yl)-5-(4-fluorophenethoxy)-6′-methyl-4-(trifluoromethyl)-[2,3′-bipyridin]-5′-yl)acetate (36 mg, 0.055 mmol) in EtOH (1.5 mL) was added sodium hydroxide (0.109 mL, 0.546 mmol) and heated at 75° C. for 24 hrs. The reaction was cooled to RT, filtered through a nylon 0.45μ frit filter and purified via preparative LC/MS. Fractions containing the desired product were combined and dried via centrifugal evaporation to afford the product (S)-2-(tert-butoxy)-2-(4′-(4,4-dimethylpiperidin-1-yl)-5-(4-fluorophenethoxy)-6′-methyl-4-(trifluoromethyl)-[2,3′-bipyridin]-5′-yl)acetic acid (24.4 mg, 0.040 mmol, 72.4% yield). ¹H NMR (500 MHz, methanol-d4) δ 8.62 (s, 1H), 8.08 (s, 1H), 7.63 (s, 1H), 7.36 (dd, J=8.4, 5.5 Hz, 2H), 7.03 (t, J=8.8 Hz, 2H), 5.66 (s, 1H), 3.18 (t, J=6.2 Hz, 2H), 2.65 (s, 3H), 1.48-1.20 (m, 4H), 1.16 (s, 9H), 0.87 (br s, 6H). 4 Protons (4 protons from methylenes closest to piperidine nitrogen) and 2 protons (benzylic protons) were not observed in HNMR. ESI-MS(+) m/z=618.2 (M+1).

### Example 245

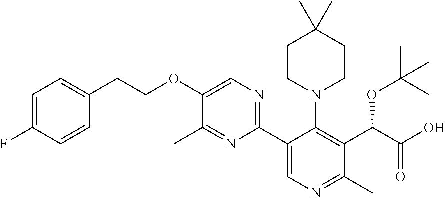 [SMILES: Cc1nc(-c2cnc(C)c([C@H](OC(C)(C)C)C(=O)O)c2N2CCC(C)(C)CC2)ncc1OCCc1ccc(F)cc1]

### (S)-2-(tert-Butoxy)-2-(4-(4, 4-dimethylpiperidin-1-yl)-5-(5-(4-fluorophenethoxy)-4-methylpyrimidin-2-yl)-2-methylpyridin-3-yl)acetic acid

To a solution of isopropyl (S)-2-(tert-butoxy)-2-(4-(4,4-dimethylpiperidin-1-yl)-5-(5-(4-fluorophenethoxy)-4-methylpyrimidin-2-yl)-2-methylpyridin-3-yl)acetate (26 mg, 0.043 mmol) in EtOH (1 mL) was added 1M sodium hydroxide (0.086 mL, 0.428 mmol) then heated at 75° C. for 24 hrs. The reaction was cooled to RT, filtered through a nylon 0.45μ frit filter and purified via preparative LCMS. Fractions containing the desired product were combined and dried via centrifugal evaporation to afford the product (S)-2-(tert-butoxy)-2-(4-(4,4-dimethylpiperidin-1-yl)-5-(5-(4-fluorophenethoxy)-4-methylpyrimidin-2-yl)-2-methylpyridin-3-yl)acetic acid (19.4 mg, 0.034 mmol, 80% yield). ¹H NMR (500 MHz, methanol-d4) δ 8.44 (s, 1H), 8.23 (s, 1H), 7.40-7.31 (m, 2H), 7.05 (t, J=8.8 Hz, 2H), 5.85 (s, 1H), 4.44 (td, J=6.4, 1.5 Hz, 2H), 3.18 (t, J=6.2 Hz, 2H), 3.02-2.79 (m, 2H), 2.64 (s, 3H), 2.46 (s, 3H), 1.43 (br s, 4H), 1.18 (s, 9H), 0.91 (s, 6H). ESI-MS(+) m/z=565.3 (M+1).

### Example 246

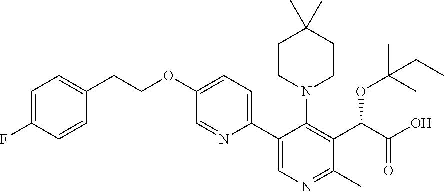 [SMILES: CCC(C)(C)O[C@H](C(=O)O)c1c(C)ncc(-c2ccc(OCCc3ccc(F)cc3)cn2)c1N1CCC(C)(C)CC1]

### (S)-2-(4′-(4, 4-Dimethylpiperidin-1-yl)-5-(4-fluorophenethoxy)-6′-methyl-[2, 3′-bipyridin]-5′-yl)-2-(tert-pentyloxy)acetic acid

To a 14 mL test tube equipped with a stir bar was added 2-(5-(4-fluorophenethoxy)pyridin-2-yl)-6-methyl-1,3,6,2-dioxazaborocane-4,8-dione (59.5 mg, 0.160 mmol), isopropyl (S)-2-(5-bromo-4-(4,4-dimethylpiperidin-1-yl)-2-methylpyridin-3-yl)-2-(tert-pentyloxy)acetate (50 mg, 0.107 mmol), palladium tetrakis (24.62 mg, 0.021 mmol), diacetoxycopper (9.67 mg, 0.053 mmol) and anhydrous tribasic potassium phosphate (finely ground, 113 mg, 0.533 mmol). The test tube was sealed with a rubber septum, then placed under N₂ atm. To the test tube was added a degassed (N₂ sparging for 5 min) solution of diethanolamine (11.2 mg, 0.107 mmol) in DMF (1.0 mL). The test tube was placed in a 100° C. heating block with stirring for 18 h. To the test tube was added water (4 mL). The mixture was extracted with EtOAc (2×5 mL). The organic phase was dried over MgSO4; filtered; then concentrated in vacuo. The resulting residue was dissolved in a min of acetone, then concentrated onto Celite in vacuo. The resulting powder was subjected to SiO2 purification (hexanes:EtOAc) to afford the desired intermediate, isopropyl (S)-2-(4′-(4,4-dimethylpiperidin-1-yl)-5-(4-fluorophenethoxy)-6′-methyl-[2,3′-bipyridin]-5′-yl)-2-(tert-pentyloxy)acetate. This material was transferred to a 1 dram vial. To the vial was added a stir bar and ethanol (2 mL), then aq. sodium hydroxide (5.0 M, 0.213 mL, 1.065 mmol). The vial was capped, then placed in a 90° C. heating block with stirring for 3.5 h. The reaction mixture was cooled to r.t., then filtered through a 0.4 micron syringe filter. The filtrate was directly subjected to HPLC purification with the following conditions: Column: XBridge C18, 19×200 mm, 5-m particles; Mobile Phase A: 5:95 acetonitrile:water with 10-mM ammonium acetate; Mobile Phase B: 95:5 acetonitrile:water with 10-mM ammonium acetate; Gradient: 35-75% B over 20 minutes, then a 4-minute hold at 100% B; Flow: 20 mL/min. This purification afforded the desired compound, (S)-2-(4′-(4,4-dimethylpiperidin-1-yl)-5-(4-fluorophenethoxy)-6′-methyl-[2,3′-bipyridin]-5′-yl)-2-(tert-pentyloxy)acetic acid (15.6 mg, 27% yield, 563.71% purity). LCMS Method 2: retention time=2.04 min.; observed ion=564.4. 1H NMR (500 MHz, METHANOL-d4) Shift 8.34 (d, J=2.7 Hz, 1H), 8.14 (s, 1H), 7.51 (dd, J=8.5, 3.1 Hz, 1H), 7.42 (d, J=8.5 Hz, 1H), 7.35 (dd, J=8.4, 5.3 Hz, 2H), 7.04 (t, J=8.3 Hz, 2H), 5.92 (s, 1H), 4.36 (t, J=6.6 Hz, 2H), 3.14 (t, J=6.6 Hz, 2H), 2.93 (br s, 2H), 2.81 (br s, 2H), 2.65 (s, 3H), 1.63-1.47 (m, 2H), 1.45-1.39 (m, 4H), 1.20 (s, 3H), 1.13 (s, 3H), 0.89 (s, 6H), 0.79 (t, J=7.5 Hz, 3H).

### Example 247

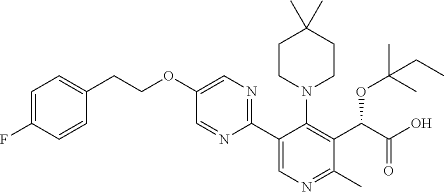 [SMILES: CCC(C)(C)O[C@H](C(=O)O)c1c(C)ncc(-c2ncc(OCCc3ccc(F)cc3)cn2)c1N1CCC(C)(C)CC1]

### (S)-2-(4-(4, 4-Dimethylpiperidin-1-yl)-5-(5-(4-fluorophenethoxy)pyrimidin-2-yl)-2-methylpyridin-3-yl)-2-(tert-pentyloxy)acetic acid

To a solution of isopropyl (S)-2-(4-(4,4-dimethylpiperidin-1-yl)-5-(5-(4-fluorophenethoxy)pyrimidin-2-yl)-2-methylpyridin-3-yl)-2-(tert-pentyloxy)acetate (21 mg, 0.035 mmol) in EtOH (1.5 mL) and Water (0.150 mL) was added lithium hydroxide monohydrate (14.52 mg, 0.346 mmol) and heated at 75° C. for 24 hrs. After 24 hrs, sodium hydroxide (0.069 mL, 0.346 mmol) was added and stirred for 3 more hrs. The reaction was cooled to RT, filtered through a nylon 0.45 g frit filter and purified via preparative LC/MS. Fractions containing the desired product were combined and dried via centrifugal evaporation to afford the product (S)-2-(4-(4,4-dimethylpiperidin-1-yl)-5-(5-(4-fluorophenethoxy)pyrimidin-2-yl)-2-methylpyridin-3-yl)-2-(tert-pentyloxy)acetic acid (12.4 mg, 0.022 mmol, 63.4% yield). ¹H NMR (500 MHz, methanol-d4) δ 8.61 (s, 2H), 8.38-8.22 (m, 1H), 7.36 (dd, J=8.4, 5.5 Hz, 2H), 7.09-6.98 (m, 2H), 6.03-5.88 (m, 1H), 4.59-4.36 (m, 2H), 3.16 (t, J=6.4 Hz, 2H), 3.02-2.74 (m, 4H), 2.65 (s, 3H), 1.63-1.49 (m, 2H), 1.44 (br s, 4H), 1.20 (s, 3H), 1.14 (s, 3H), 0.92 (s, 6H), 0.78 (t, J=7.5 Hz, 3H). ESI-MS(+) m/z=565.3 (M+1).

### Example 248

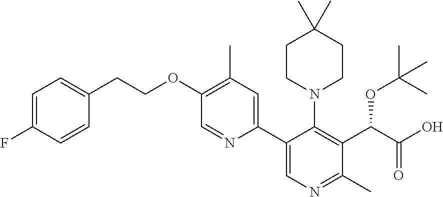 [SMILES: Cc1cc(-c2cnc(C)c([C@H](OC(C)(C)C)C(=O)O)c2N2CCC(C)(C)CC2)ncc1OCCc1ccc(F)cc1]

### (S)-2-(tert-Butoxy)-2-(4′-(4, 4-dimethylpiperidin-1-yl)-5-(4-fluorophenethoxy)-4, 6′-dimethyl-[2, 3′-bipyridin]-5′-yl)acetic acid

To a solution of isopropyl (S)-2-(tert-butoxy)-2-(4′-(4,4-dimethylpiperidin-1-yl)-5-(4-fluorophenethoxy)-4,6′-dimethyl-[2,3′-bipyridin]-5′-yl)acetate (12 mg, 0.020 mmol) in EtOH (1 mL) and added sodium hydroxide (0.040 mL, 0.198 mmol) then heated at 75° C. for 24 hrs. The reaction was cooled to RT, filtered through a nylon 0.45 g frit filter and purified via preparative LC/MS. Fractions containing the desired product were combined and dried via centrifugal evaporation to afford the product (S)-2-(tert-butoxy)-2-(4′-(4,4-dimethylpiperidin-1-yl)-5-(4-fluorophenethoxy)-4,6′-dimethyl-[2, 3′-bipyridin]-5′-yl)acetic acid (8.5 mg, 0.015 mmol, 76% yield). ¹H NMR (500 MHz, methanol-d4) δ 8.23 (s, 1H), 8.07 (s, 1H), 7.35 (dd, J=8.4, 5.5 Hz, 2H), 7.28 (s, 1H), 7.07-6.99 (m, 2H), 5.83-5.74 (m, 1H), 4.45-4.31 (m, 2H), 3.16 (t, J=6.4 Hz, 2H), 3.10-2.65 (m, 4H), 2.64 (s, 3H), 2.26 (s, 3H), 1.40 (br s, 4H), 1.18 (s, 9H), 0.87 (s, 6H). ESI-MS(+) m/z=564.4 (M+1).

### Example 249

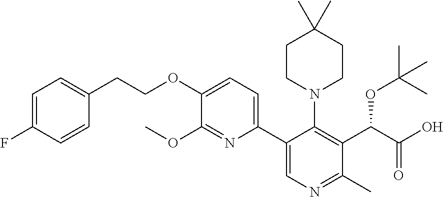 [SMILES: COc1nc(-c2cnc(C)c([C@H](OC(C)(C)C)C(=O)O)c2N2CCC(C)(C)CC2)ccc1OCCc1ccc(F)cc1]

### (S)-2-(tert-Butoxy)-2-(4′-(4, 4-dimethylpiperidin-1-yl)-5-(4-fluorophenethoxy)-6-methoxy-6′-methyl-[2, 3′-bipyridin]-5′-yl)acetic acid

To a solution of isopropyl (S)-2-(tert-butoxy)-2-(4′-(4,4-dimethylpiperidin-1-yl)-5-(4-fluorophenethoxy)-6-methoxy-6′-methyl-[2,3′-bipyridin]-5′-yl)acetate (77 mg, 0.124 mmol) in EtOH (1 mL) was added sodium hydroxide (0.248 mL, 1.238 mmol) then heated at 75° C. for 24 hrs. The reaction was cooled to RT, filtered through a nylon 0.45μ frit filter and purified via preparative LC/MS. Fractions containing the desired product were combined and dried via centrifugal evaporation. Purification afforded the product (S)-2-(tert-butoxy)-2-(4′-(4,4-dimethylpiperidin-1-yl)-5-(4-fluorophenethoxy)-6-methoxy-6′-methyl-[2,3′-bipyridin]-5′-yl)acetic acid (15.8 mg, 0.027 mmol, 21.79% yield). ¹H NMR (500 MHz, methanol-d4) δ 8.12 (s, 1H), 7.39-7.30 (m, 3H), 7.02 (t, J=8.8 Hz, 2H), 6.96 (d, J=8.1 Hz, 1H), 5.88 (s, 1H), 4.30 (t, J=6.6 Hz, 2H), 4.00 (s, 3H), 3.12 (t, J=6.6 Hz, 2H), 3.09-2.76 (m, 4H), 2.68-2.61 (m, 3H), 1.45 (br s, 4H), 1.21 (s, 9H), 0.91 (s, 6H). ESI-MS(+) m/z=580.4 (M+1).

### Example 250

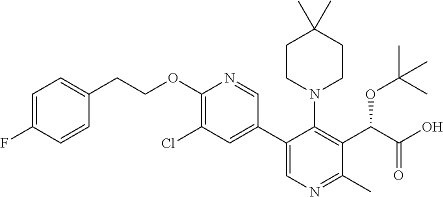 [SMILES: Cc1ncc(-c2cnc(OCCc3ccc(F)cc3)c(Cl)c2)c(N2CCC(C)(C)CC2)c1[C@H](OC(C)(C)C)C(=O)O]

### (S)-2-(tert-Butoxy)-2-(5′-chloro-4-(4, 4-dimethylpiperidin-1-yl)-6′-(4-fluorophenethoxy)-6-methyl-[3, 3′-bipyridin]-5-yl)acetic acid

A mixture of 2-(4-fluorophenyl)ethan-1-ol (0.032 g, 0.227 mmol, 5 equiv), 60% NaH (9.09 mg, 0.227 mmol, 5 equiv), and isopropyl (S)-2-(tert-butoxy)-2-(5′-chloro-4-(4,4-dimethylpiperidin-1-yl)-6′-fluoro-6-methyl-[3,3′-bipyridin]-5-yl)acetate (0.023 g, 0.045 mmol, 1 equiv) in THF (1.5 mL) was stirred for 1 h. After complete addition, 5 M NaOH (0.167 mL, 0.837 mmol, 10 equiv) was added and the mixture was heated at 80° C. for 2 h. Upon cooling to ambient temperature, the reaction was filtered and purified by reverse phase preparative HPLC to afford the product (9.5 mg, 36%). ¹H NMR (500 MHz, DMSO-d₆) δ 8.12-8.03 (m, 2H), 7.87 (d, J=2.2 Hz, 1H), 7.41-7.31 (m, 2H), 7.15-7.06 (m, 2H), 5.75 (s, 1H), 4.62 (t, J=6.8 Hz, 2H), 3.09 (t, J=6.6 Hz, 2H), 1.37-1.25 (m, 3H), 1.14 (s, 10H), 0.94-0.75 (m, 6H) [note: some piperidine protons not seen]. LCMS (M+1): 584.1.

### Example 251

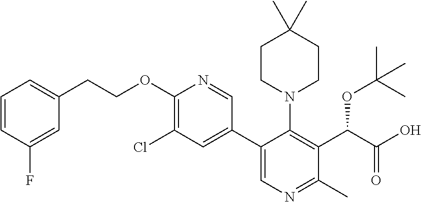 [SMILES: Cc1ncc(-c2cnc(OCCc3cccc(F)c3)c(Cl)c2)c(N2CCC(C)(C)CC2)c1[C@H](OC(C)(C)C)C(=O)O]

### (S)-2-(tert-Butoxy)-2-(5′-chloro-4-(4, 4-dimethylpiperidin-1-yl)-6′-(3-fluorophenethoxy)-6-methyl-[3, 3′-bipyridin]-5-yl)acetic acid

A mixture of 2-(3-fluorophenyl)ethan-1-ol (0.032 g, 0.227 mmol, 5 equiv), 60% NaH (9.09 mg, 0.227 mmol, 5 equiv), and isopropyl (S)-2-(tert-butoxy)-2-(5′-chloro-4-(4,4-dimethylpiperidin-1-yl)-6′-fluoro-6-methyl-[3,3′-bipyridin]-5-yl)acetate (0.023 g, 0.045 mmol, 1 equiv) in THF (1.5 mL) was stirred for 1 h. After complete addition, 5 M NaOH (0.167 mL, 0.837 mmol, 10 equiv) was added and the mixture was heated at 80° C. for 2 h. Upon cooling to ambient temperature, the reaction was filtered and purified by reverse phase preparative HPLC to give the product (17.2 mg, 64%). ¹H NMR (500 MHz, DMSO-d₆) δ 8.15-7.99 (m, 2H), 7.87 (d, J=2.2 Hz, 1H), 7.41-6.92 (m, 4H), 5.74 (s, 1H), 4.65 (t, J=6.6 Hz, 2H), 2.52 (br s, 2H), 1.32 (br s, 3H), 1.13 (s, 9H), 1.00-0.74 (m, 6H), 0.80-0.72 (m, 1H). LCMS (M+1): 584.2.

### Example 252

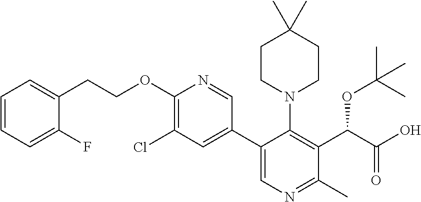 [SMILES: Cc1ncc(-c2cnc(OCCc3ccccc3F)c(Cl)c2)c(N2CCC(C)(C)CC2)c1[C@H](OC(C)(C)C)C(=O)O]

### (S)-2-(tert-Butoxy)-2-(5′-chloro-4-(4, 4-dimethylpiperidin-1-yl)-6′-(2-fluorophenethoxy)-6-methyl-[3, 3′-bipyridin]-5-yl)acetic acid

A mixture of 2-(2-fluorophenyl)ethan-1-ol (0.032 g, 0.227 mmol, 5 equiv), 60% NaH (9.09 mg, 0.227 mmol, 5 equiv), and isopropyl (S)-2-(tert-butoxy)-2-(5′-chloro-4-(4,4-dimethylpiperidin-1-yl)-6′-fluoro-6-methyl-[3,3′-bipyridin]-5-yl)acetate (0.023 g, 0.045 mmol, 1 equiv) in THF (1.5 mL) was stirred for 1 h. After addition, 5 M NaOH (0.167 mL, 0.837 mmol, 10 equiv) was added and the mixture was heated at 80° C. for 2 h. Upon cooling to ambient temperature, the reaction was filtered and purified by reverse phase preparative HPLC to afford the product (1.9 mg, 7%). ¹H NMR (500 MHz, DMSO-d₆) δ 8.12-8.01 (m, 2H), 7.88 (s, 1H), 7.44-7.08 (m, 4H), 5.72 (s, 1H), 4.72-4.60 (m, 2H), 2.52 (br s, 2H), 1.36-1.24 (m, 3H), 1.13 (s, 9H), 0.92-0.74 (m, 6H). LCMS (M+1): 584.2.

### Example 253

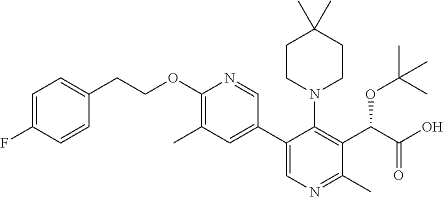 [SMILES: Cc1cc(-c2cnc(C)c([C@H](OC(C)(C)C)C(=O)O)c2N2CCC(C)(C)CC2)cnc1OCCc1ccc(F)cc1]

### (S)-2-(tert-Butoxy)-2-(4-(4, 4-dimethylpiperidin-1-yl)-6′-(4-fluorophenethoxy)-5′,6-dimethyl-[3,3′-bipyridin]-5-yl)acetic acid

A mixture of 2-(4-fluorophenyl)ethan-1-ol (0.033 g, 0.237 mmol, 5 equiv), 60% NaH (9.47 mg, 0.237 mmol, 5 equiv), and isopropyl (S)-2-(tert-butoxy)-2-(4-(4,4-dimethylpiperidin-1-yl)-6′-fluoro-5′,6-dimethyl-[3,3′-bipyridin]-5-yl)acetate (0.023 g, 0.047 mmol, 1 equiv) in THF (1.5 mL) was stirred for 1 h. After complete addition, 5 M NaOH (0.167 mL, 0.837 mmol, 10 equiv) was added and the mixture was heated at 80° C. for 2 h. After cooling to ambient temperature, the reaction was filtered and purified by reverse phase preparative HPLC to provide the product (8.8. mg, 33%). ¹H NMR (500 MHz, DMSO-d₆) δ 8.06-8.00 (m, 1H), 7.92 (d, J=2.2 Hz, 1H), 7.49 (d, J=1.5 Hz, 1H), 7.39-7.29 (m, 2H), 7.11 (t, J=9.0 Hz, 2H), 5.78 (s, 1H), 4.55 (t, J=6.6 Hz, 2H), 2.16 (s, 3H), 1.40-1.25 (m, 3H), 1.13 (s, 10H), 0.91-0.73 (m, 6H). LCMS (M+1): 564.3.

### Example 254

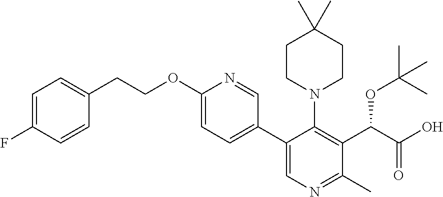 [SMILES: Cc1ncc(-c2ccc(OCCc3ccc(F)cc3)nc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-6′-(4-fluorophenethoxy)-6-methyl-[3,3′-bipyridin]-5-yl)acetic acid

A mixture of 2-(4-fluorophenyl)ethan-1-ol (0.031 g, 0.223 mmol, 5 equiv), 60% NaH (8.90 mg, 0.223 mmol, 5 equiv) and isopropyl (S)-2-(tert-butoxy)-2-(4-(4,4-dimethylpiperidin-1-yl)-6′-fluoro-6-methyl-[3,3′-bipyridin]-5-yl)acetate (0.021 g, 0.045 mmol, 1 equiv) in THF (1.5 mL) was stirred at ambient temperature for 1 h. Upon complete addition, 5 M NaOH (0.167 mL, 0.837 mmol, 10 equiv) was added and the mixture was heated at 80° C. for 2 h. The reaction was allowed to cool to ambient temperature, filtered, and purified by preparative reverse phase HPLC to provide the product (6.9 mg, 28%). ¹H NMR (500 MHz, DMSO-d₆) δ 8.14-7.96 (m, 2H), 7.65 (dd, J=8.3, 2.4 Hz, 1H), 7.34 (dd, J=8.4, 5.5 Hz, 2H), 7.18-7.07 (m, 2H), 6.88 (d, J=8.4 Hz, 1H), 5.79 (s, 1H), 4.60-4.46 (m, 2H), 1.39-1.25 (m, 3H), 1.20-1.09 (m, 10H), 0.94-0.74 (m, 6H) [note: some piperidine protons not observed]. LCMS (M+1): 550.2.

### Example 255

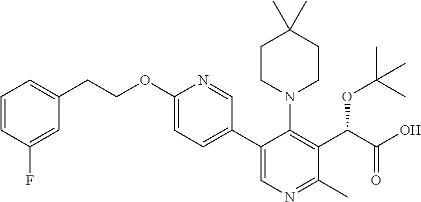 [SMILES: Cc1ncc(-c2ccc(OCCc3cccc(F)c3)nc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-6′-(3-fluorophenethoxy)-6-methyl-[3,3′-bipyridin]-5-yl)acetic acid

A mixture of 2-(3-fluorophenyl)ethan-1-ol (0.031 g, 0.223 mmol, 5 equiv), 60% NaH (8.90 mg, 0.223 mmol, 5 equiv), and isopropyl (S)-2-(tert-butoxy)-2-(4-(4,4-dimethylpiperidin-1-yl)-6′-fluoro-6-methyl-[3,3′-bipyridin]-5-yl)acetate (0.021 g, 0.045 mmol, 1 equiv) in THF (1.5 mL) was stirred at ambient temperature for 1 h. Upon complete addition, 5 M NaOH (0.089 mL, 0.445 mmol, 10 equiv) was added and the mixture was heated at 80° C. for 2 h. The reaction was allowed to cool to ambient temperature, filtered, and purified by preparative reverse phase HPLC to provide the product (10 mg, 40%). ¹H NMR (500 MHz, DMSO-d₆) δ 8.18-7.97 (m, 2H), 7.73-7.60 (m, 1H), 7.40-7.29 (m, 1H), 7.21-6.94 (m, 3H), 6.93-6.83 (m, 1H), 5.86-5.75 (m, 1H), 4.62-4.50 (m, 2H), 3.14-3.02 (m, 1H), 1.38-1.24 (m, 3H), 1.18-1.02 (m, 10H), 0.91-0.70 (m, 6H). LCMS (M+1): 550.3.

### Example 256

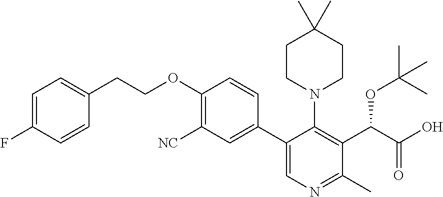 [SMILES: Cc1ncc(-c2ccc(OCCc3ccc(F)cc3)c(C#N)c2)c(N2CCC(C)(C)CC2)c1[C@H](OC(C)(C)C)C(=O)O]

### (S)-2-(tert-Butoxy)-2-(5-(3-cyano-4-(4-fluorophenethoxy)phenyl)-4-(4, 4-dimethylpiperidin-1-yl)-2-methylpyridin-3-yl)acetic acid

A mixture of (S)-2-(5-bromo-4-(4,4-dimethylpiperidin-1-yl)-2-methylpyridin-3-yl)-2-(tert-butoxy)acetic acid (0.04 g, 0.097 mmol, 1 equiv), 2-(4-fluorophenethoxy)-5-(4,4,5,5-tetramethyl-1,3,2-dioxaborolan-2-yl)benzonitrile (0.053 g, 0.145 mmol, 1.5 equiv), SPhos (7.95 mg, 0.019 mmol, 0.2 equiv), palladium(II) acetate (2.173 mg, 9.68 μmol, 0.1 equiv), and 2 M K₃PO₄ (0.145 ml, 0.290 mmol, 3 equiv) in dioxane (1 mL) was heated at 90° C. for 4 h. After cooling to ambient temperature, the reaction mixture was filtered through celite/Na₂SO₄ eluting with ethyl acetate and concentrated in vacuo. The crude product was purified by reverse phase preparative HPLC to provide the product (4 mg, 7%). ¹H NMR (500 MHz, DMSO-d₆) δ 8.06-7.99 (m, 1H), 7.65 (d, J=1.8 Hz, 1H), 7.59-7.55 (m, 1H), 7.45-7.32 (m, 3H), 7.12 (t, J=8.8 Hz, 2H), 5.79-5.69 (m, 1H), 4.48 (br d, J=4.4 Hz, 2H), 1.40-1.25 (m, 3H), 1.17-1.08 (m, 10H), 0.91-0.75 (m, 6H). LCMS (M+1): 574.4.

### Example 257

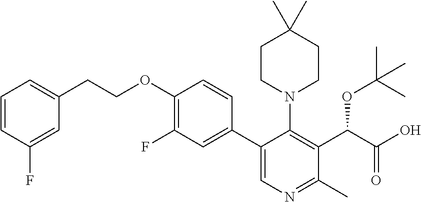 [SMILES: Cc1ncc(-c2ccc(OCCc3cccc(F)c3)c(F)c2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(3-fluoro-4-(3-fluorophenethoxy)phenyl)-2-methylpyridin-3-yl)acetic acid

A mixture of isopropyl (S)-2-(5-bromo-4-(4,4-dimethylpiperidin-1-yl)-2-methylpyridin-3-yl)-2-(tert-butoxy)acetate (0.065 g, 0.143 mmol, 1 equiv), 2-(3-fluoro-4-(3-fluorophenethoxy)phenyl)-4,4,5,5-tetramethyl-1,3,2-dioxaborolane (0.077 g, 0.214 mmol, 1.5 equiv), Pd(dppf)Cl₂ (10 mg, 0.014 mmol, 0.1 equiv), and 2 M K₃PO₄ (0.43 ml, 0.856 mmol, 6 equiv) in dioxane (1.4 mL) was heated at 80° C. for 2 h. After cooling to ambient temperature, the reaction mixture was diluted with ethyl acetate and washed with brine. The organic layer was dried (Na₂SO₄) and concentrated in vacuo. The residue was taken up in ethanol (2 mL) and 10 M NaOH (0.2 mL) was added. The mixture was heated at 80° C. for 18 h. After cooling to ambient temperature, the mixture was filtered and purified by reverse phase preparative HPLC to provide the product (20 mg, 23%). ¹H NMR (500 MHz, DMSO-d₆) δ 8.07 (s, 1H), 7.39-7.33 (m, 1H), 7.29 (t, J=8.8 Hz, 1H), 7.23-7.15 (m, 3H), 7.09-7.00 (m, 2H), 5.82 (s, 1H), 4.37 (td, J=6.7, 3.9 Hz, 2H), 3.12 (t, J=6.6 Hz, 2H), 2.52 (br s, 3H), 1.33 (br d, J=5.5 Hz, 4H), 1.14 (s, 9H), 0.83 (s, 6H) [note: some piperidine protons not visible]. LCMS (M+1): 567.3.

### Example 258

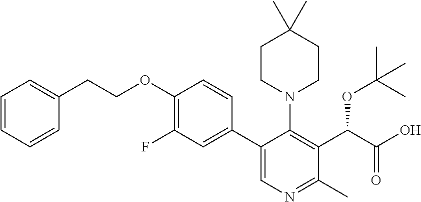 [SMILES: Cc1ncc(-c2ccc(OCCc3ccccc3)c(F)c2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(3-fluoro-4-phenethoxyphenyl)-2-methylpyridin-3-yl)acetic acid

A solution of isopropyl (S)-2-(5-bromo-4-(4,4-dimethylpiperidin-1-yl)-2-methylpyridin-3-yl)-2-(tert-butoxy)acetate (0.064 g, 0.141 mmol, 1 equiv), 2-(3-fluoro-4-phenethoxyphenyl)-4,4,5,5-tetramethyl-1,3,2-dioxaborolane (0.072 g, 0.211 mmol, 1.5 equiv), Pd(dppf)Cl₂ (10 mg, 0.014 mmol, 0.1 equiv), and 2 M K₃PO₄ (0.42 ml, 0.843 mmol, 6 equiv) in dioxane (1.4 mL) was heated at 80° C. for 2 h. After cooling to ambient temperature, the reaction mixture was diluted with ethyl acetate and washed with brine.

The organic layer was dried (Na₂SO₄) and concentrated in vacuo. The residue was taken up in ethanol (2 mL) and 10 M NaOH (0.2 mL) was added. The mixture was heated at 80° C. for 18 h. After cooling to ambient temperature, the mixture was filtered and purified by reverse phase preparative HPLC to provide the product (27 mg, 34%). ¹H NMR (500 MHz, DMSO-d₆) δ 8.06 (s, 1H), 7.38-7.22 (m, 6H), 7.18 (dd, J=12.1, 2.2 Hz, 1H), 7.06 (br d, J=7.7 Hz, 1H), 5.83 (s, 1H), 4.35 (td, J=6.7, 4.6 Hz, 2H), 3.09 (t, J=6.8 Hz, 2H), 2.51 (s, 3H), 1.32 (br d, J=2.6 Hz, 4H), 1.14 (s, 9H), 0.83 (s, 6H) [note: some piperidine protons not visible]. LCMS (M+1): 549.2.

### Example 259

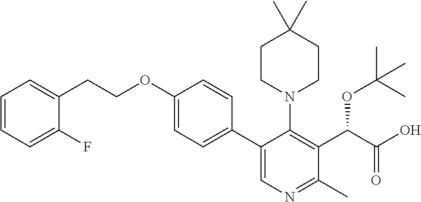 [SMILES: Cc1ncc(-c2ccc(OCCc3ccccc3F)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(4-(2-fluorophenethoxy)phenyl)-2-methylpyridin-3-yl)acetic acid

A mixture of isopropyl (S)-2-(5-bromo-4-(4,4-dimethylpiperidin-1-yl)-2-methylpyridin-3-yl)-2-(tert-butoxy)acetate (0.04 g, 0.088 mmol, 1 equiv), 2-(4-(3-fluorophenethoxy)phenyl)-4,4,5,5-tetramethyl-1,3,2-dioxaborolane (0.045 g, 0.132 mmol, 1.5 equiv), Sphos (7.21 mg, 0.018 mmol, 0.2 equiv), palladium(II) acetate (1.972 mg, 8.78 μmol, 0.1 equiv), and 2 M K₃PO₄ (0.132 ml, 0.263 mmol, 3 equiv) in dioxane was heated at 90° C. for 4 h. After cooling to ambient temperature, the reaction mixture was filtered through celite/Na₂SO₄ eluting with ethyl acetate. The filtrate was concentrated in vacuo. The residue was taken up in ethanol (1 mL) and 5 M NaOH (0.176 ml, 0.878 mmol, 10 equiv) was added. The mixture was heated at 90° C. for 4 h. After cooling to ambient temperature, the mixture was filtered and purified by reverse phase preparative HPLC to provide the product (23 mg, 46%). ¹H NMR (500 MHz, DMSO-d₆) δ 8.12-8.04 (m, 1H), 7.42-7.32 (m, 1H), 7.27-7.14 (m, 4H), 7.09-7.01 (m, 3H), 5.86-5.80 (m, 1H), 4.36-4.23 (m, 2H), 3.13-3.07 (m, 1H), 1.32 (br d, J=4.8 Hz, 3H), 1.14 (s, 10H), 0.82 (s, 6H). LCMS (M+1): 549.4.

### Example 260

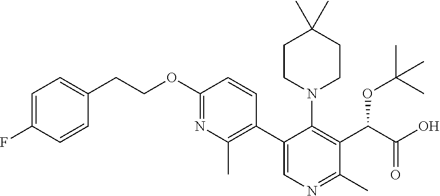 [SMILES: Cc1nc(OCCc2ccc(F)cc2)ccc1-c1cnc(C)c([C@H](OC(C)(C)C)C(=O)O)c1N1CCC(C)(C)CC1]

### (S)-2-(tert-Butoxy)-2-(4-(4, 4-dimethylpiperidin-1-yl)-6′-(4-fluorophenethoxy)-2′,6-dimethyl-[3, 3′-bipyridin]-5-yl)acetic acid

A mixture of 2-(4-fluorophenyl)ethan-1-ol (0.033 g, 0.237 mmol, 5 equiv), 60% NaH (9.47 mg, 0.237 mmol, 5 equiv), and isopropyl (S)-2-(tert-butoxy)-2-(4-(4,4-dimethylpiperidin-1-yl)-6′-fluoro-2′,6-dimethyl-[3,3′-bipyridin]-5-yl)acetate (0.023 g, 0.047 mmol, 1 equiv) in THF (1.5 mL) was stirred for 1 h. After complete addition, 5 M NaOH (0.167 mL, 0.837 mmol, 10 equiv) was added and the mixture was heated at 80° C. for 2 h. Upon cooling to ambient temperature, the reaction was filtered and purified by reverse phase preparative HPLC to provide the product (6 mg, 22%). NMR shows a 2:1 mixture of atropisomers, major isomer transcribed. ¹H NMR (500 MHz, DMSO-d₆) δ 7.98 (s, 1H), 7.40 (d, J=8.1 Hz, 1H), 7.35 (dd, J=8.4, 5.5 Hz, 2H), 7.16-7.06 (m, 2H), 6.70 (d, J=8.4 Hz, 1H), 5.84 (s, 1H), 4.56-4.47 (m, 2H), 3.10-3.00 (m, 2H), 2.50 (s, 3H), 2.21 (s, 3H), 1.35-1.19 (m, 4H), 1.14 (s, 9H), 0.76 (br s, 6H) [note: some piperidine protons not observed]. LCMS (M+1): 564.4.

### Example 261

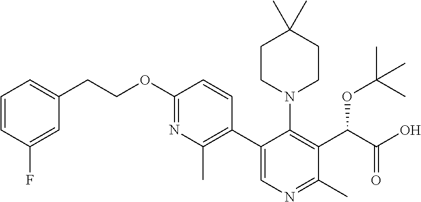 [SMILES: Cc1nc(OCCc2cccc(F)c2)ccc1-c1cnc(C)c([C@H](OC(C)(C)C)C(=O)O)c1N1CCC(C)(C)CC1]

### (S)-2-(tert-Butoxy)-2-(4-(4, 4-dimethylpiperidin-1-yl)-6′-(3-fluorophenethoxy)-2′,6-dimethyl-[3, 3′-bipyridin]-5-yl)acetic acid

A mixture of 2-(3-fluorophenyl)ethan-1-ol (0.033 g, 0.237 mmol, 5 equiv), 60% NaH (9.47 mg, 0.237 mmol, 5 equiv), and isopropyl (S)-2-(tert-butoxy)-2-(4-(4,4-dimethylpiperidin-1-yl)-6′-fluoro-2′,6-dimethyl-[3,3′-bipyridin]-5-yl)acetate (0.023 g, 0.047 mmol, 1 equiv) in THF (1.5 mL) was stirred for 1 h. Upon completion, 5 N NaOH (0.167 mL, 0.837 mmol, 10 equiv) was added and the mixture was heated at 80° C. for 2 h. After cooling to ambient temperature, the reaction was filtered and purified by reverse phase preparative HPLC to give the product (6.6 mg, 24%). NMR shows a mixture of atropisomers, major isomer transcribed. ¹H NMR (500 MHz, DMSO-d₆) δ 7.98 (s, 1H), 7.41 (d, J=8.4 Hz, 1H), 7.21-7.14 (m, J=4.8 Hz, 2H), 7.04 (td, J=8.7, 2.8 Hz, 1H), 6.71 (d, J=8.1 Hz, 1H), 5.84 (s, 1H), 4.61-4.52 (m, 2H), 3.09 (td, J=6.5, 3.1 Hz, 2H), 2.51 (s, 3H), 2.21 (s, 3H), 1.38-1.21 (m, 4H), 1.14 (s, 9H), 0.77 (br s, 6H) [note: some piperidine protons not observed]. LCMS (M+1): 564.4.

### Example 262 Example 263

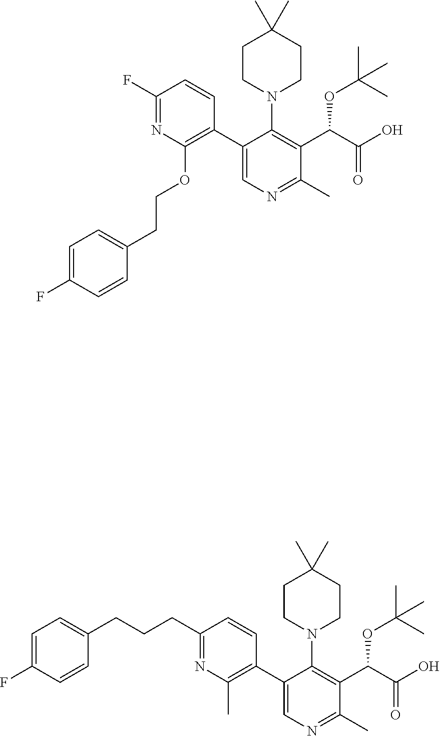 [SMILES: Cc1ncc(-c2ccc(F)nc2OCCc2ccc(F)cc2)c(N2CCC(C)(C)CC2)c1[C@H](OC(C)(C)C)C(=O)O ; Cc1nc(CCCc2ccc(F)cc2)ccc1-c1cnc(C)c([C@H](OC(C)(C)C)C(=O)O)c1N1CCC(C)(C)CC1]

### (S)-2-(tert-Butoxy)-2-(4-(4, 4-dimethylpiperidin-1-yl)-6′-fluoro-2′-(4-fluorophenethoxy)-6-methyl-[3, 3′-bipyridin]-5-yl)acetic acid and (S)-2-(tert-butoxy)-2-(4-(4, 4-dimethylpiperidin-1-yl)-2′-fluoro-6′-(4-fluorophenethoxy)-6-methyl-[3,3′-bipyridin]-5-yl)acetic acid

A mixture of 2-(4-fluorophenyl)ethan-1-ol (0.031 g, 0.225 mmol, 5 equiv), 60% NaH (8.99 mg, 0.225 mmol, 5 equiv), and isopropyl (S)-2-(tert-butoxy)-2-(4-(4,4-dimethylpiperidin-1-yl)-2′,6′-difluoro-6-methyl-[3,3′-bipyridin]-5-yl)acetate (0.022 g, 0.045 mmol, 1 equiv) in THF (1.5 mL) was stirred for 1 h. Upon completion, 5 M NaOH (0.167 mL, 0.837 mmol, 10 equiv) was added and the mixture was heated at 80° C. for 2 h. After cooling to ambient temperature, the reaction was filtered and purified by reverse phase preparative HPLC to give two regioisomers of product (4.6 mg, 17%) and (3.4 mg, 13%). Isomer 1 (assigned as displacement of 2-F): LCMS (M+1): 568.2. Isomer 2 (assigned as displacement of 6-F): ¹H NMR (500 MHz, DMSO-d₆) δ 8.21 (br s, 1H), 7.92-7.81 (m, J=9.5, 4.8 Hz, 1H), 7.35 (dd, J=8.4, 5.5 Hz, 2H), 7.12 (t, J=9.0 Hz, 2H), 6.88 (d, J=8.4 Hz, 1H), 5.70 (br d, J=1.5 Hz, 1H), 4.51 (t, J=6.6 Hz, 2H), 3.07 (t, J=6.6 Hz, 2H), 2.58 (s, 3H), 1.44-1.25 (m, 4H), 1.15 (s, 9H), 0.84 (s, 6H) [note: some piperidine protons not observed]. LCMS (M+1): 568.3.

### Example 264

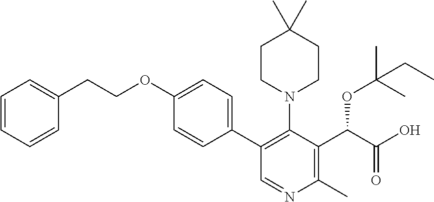 [SMILES: CCC(C)(C)O[C@H](C(=O)O)c1c(C)ncc(-c2ccc(OCCc3ccccc3)cc2)c1N1CCC(C)(C)CC1]

### (S)-2-(4-(4, 4-Dimethylpiperidin-1-yl)-2-methyl-5-(4-phenethoxyphenyl)pyridin-3-yl)-2-(tert-pentyloxy)acetic acid

A mixture of isopropyl (S)-2-(5-bromo-4-(4,4-dimethylpiperidin-1-yl)-2-methylpyridin-3-yl)-2-(tert-pentyloxy)acetate (20 mg, 0.043 mmol), 4,4,5,5-tetramethyl-2-(4-phenethoxyphenyl)-1,3,2-dioxaborolane (21 mg, 0.064 mmol), Sphos Pd G3 (3.3 mg, 4.3 μmol), and potassium phosphate tribasic (27 mg, 0.13 mmol) in 1,4-Dioxane (0.7 ml) and Water (0.1 ml) the reaction vessel capped under positive pressure of N2. The reaction was heated at 80° C. for 1 h. The reaction was concentrated, adsorbed onto celite and was purified on silica gel (Biotage, EtOAc/hexanes gradient, 0-100% over 10 CVs) to afford the ester intermediate which was taken up in EtOH (1.5 ml) and 5N NaOH (10 equiv) was added and the reaction was stirred at 100° C. for 3 hrs and then the mixture was then cooled and purified on C18 Prep HPLC to afford the expected carboxylic acid: (S)-2-(4-(4,4-dimethylpiperidin-1-yl)-2-methyl-5-(4-phenethoxyphenyl)pyridin-3-yl)-2-(tert-pentyloxy)acetic acid (14.9 mg, 64% yield). LCMS Method 2: retention time=2.29 min.; observed ion=545.4. ¹H NMR (500 MHz, DMSO-d6) δ 8.00 (s, 1H), 7.36-7.30 (m, 5H), 7.26-7.18 (m, 4H), 7.03 (d, J=7.4 Hz, 2H), 5.81 (s, 1H), 4.31-4.23 (m, 2H), 3.07 (t, J=6.8 Hz, 2H), 1.56-1.36 (m, 4H), 1.31 (br s, 3H), 1.12 (s, 4H), 1.04 (s, 4H), 0.82 (br s, 7H), 0.72 (t, J=7.5 Hz, 4H).

### Example 265

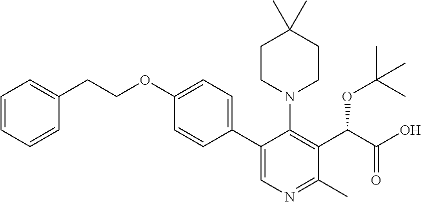 [SMILES: Cc1ncc(-c2ccc(OCCc3ccccc3)cc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-2-methyl-5-(4-phenethoxyphenyl)pyridin-3-yl)acetic acid

A mixture of isopropyl (S)-2-(5-bromo-4-(4,4-dimethylpiperidin-1-yl)-2-methylpyridin-3-yl)-2-(tert-butoxy)acetate (30 mg, 0.066 mmol), 4,4,5,5-tetramethyl-2-(4-phenethoxyphenyl)-1,3,2-dioxaborolane (32.0 mg, 0.099 mmol), Sphos Pd G3 (5.13 mg, 6.59 μmol), and potassium phosphate tribasic (41.9 mg, 0.198 mmol) in 1,4-Dioxane (1 ml) and Water (0.2 ml) the reaction vessel capped under positive pressure of N2. The reaction was heated at 80° C. for 1 h. The reaction was concentrated, adsorbed onto celite and was purified on silica gel (Biotage, EtOAc/hexanes gradient, 0-100% over 10 CVs) to give the ester intermediate which was subjected to hydrolysis conditions (1.5 mL EtOH, 0.1 mL of 5N NaOH, 100 C for 3 hrs.) The resulting carboxylic acid was purified on C18 to afford (S)-2-(tert-butoxy)-2-(4-(4,4-dimethylpiperidin-1-yl)-2-methyl-5-(4-phenethoxyphenyl)pyridin-3-yl)acetic acid (9.5 mg, 27% yield) LCMS Method 1: retention time=2.27 min.; observed ion=531.3. ¹H NMR (500 MHz, DMSO-d6) δ 7.18 (s, 1H), 6.50-6.30 (m, 5H), 6.21 (br d, J=8.4 Hz, 2H), 5.89 (br dd, J=17.2, 8.1 Hz, 3H), 4.33 (br d, J=6.6 Hz, 1H), 3.46 (br d, J=17.2 Hz, 2H), 2.19 (s, 3H), 2.11-2.03 (m, 6H), 2.00 (br s, 3H), 1.95 (br s, 3H), 0.22-0.06 (m, 12H).

### Example 266

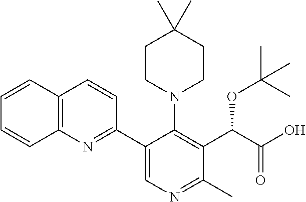 [SMILES: Cc1ncc(-c2ccc3ccccc3n2)c(N2CCC(C)(C)CC2)c1[C@H](OC(C)(C)C)C(=O)O]

### (S)-2-(tert-Butoxy)-2-(4-(4, 4-dimethylpiperidin-1-yl)-2-methyl-5-(quinolin-2-yl)pyridin-3-yl)acetic acid

To a solution of isopropyl (S)-2-(tert-butoxy)-2-(4-(4,4-dimethylpiperidin-1-yl)-2-methyl-5-(quinolin-2-yl)pyridin-3-yl)acetate (74 mg, 0.147 mmol) in EtOH (1.5 mL) was added sodium hydroxide (0.294 mL, 1.469 mmol) then heated at 75° C. for 24 hrs. The reaction was cooled to RT, filtered through a nylon 0.45μ frit filter and purified via preparative LC/MS. Fractions containing the desired product were combined and dried via centrifugal evaporation to afford the product (S)-2-(tert-butoxy)-2-(4-(4,4-dimethylpiperidin-1-yl)-2-methyl-5-(quinolin-2-yl)pyridin-3-yl)acetic acid (14.5 mg, 0.031 mmol, 21.38% yield). ¹H NMR (500 MHz, DMSO-d6) δ 8.49 (d, J=8.4 Hz, 1H), 8.30 (s, 1H), 8.05 (d, J=8.8 Hz, 2H), 7.82 (ddd, J=8.3, 6.9, 1.3 Hz, 1H), 7.70 (d, J=8.4 Hz, 1H), 7.67-7.62 (m, 1H), 5.91 (s, 1H), 2.55 (s, 3H), 1.41-1.20 (m, 4H), 1.16 (s, 9H), 0.84-0.62 (m, 6H) 4 protons (4 from 2 methylenes closest to piperidine nitrogen) were not observed in the HNMR spectrum. ESI-MS(+) m/z=462.2 (M+1).

### Example 267

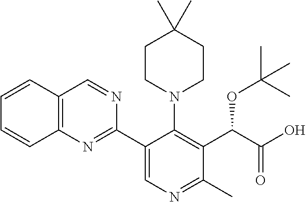 [SMILES: Cc1ncc(-c2ncc3ccccc3n2)c(N2CCC(C)(C)CC2)c1[C@H](OC(C)(C)C)C(=O)O]

### (S)-2-(tert-Butoxy)-2-(4-(4, 4-dimethylpiperidin-1-yl)-2-methyl-5-(quinazolin-2-yl)pyridin-3-yl)acetic acid

To a solution of isopropyl (S)-2-(tert-butoxy)-2-(4-(4,4-dimethylpiperidin-1-yl)-2-methyl-5-(quinazolin-2-yl)pyridin-3-yl)acetate (13 mg, 0.026 mmol) in EtOH (1.5 mL) was added sodium hydroxide (0.052 mL, 0.258 mmol) and heated at 75° C. for 24 hrs. The reaction was cooled to RT, filtered through a nylon 0.45 g frit filter and purified via preparative LC/MS. Fractions containing the desired product were combined and dried via centrifugal evaporation to afford the product (S)-2-(tert-butoxy)-2-(4-(4,4-dimethylpiperidin-1-yl)-2-methyl-5-(quinazolin-2-yl)pyridin-3-yl)acetic acid (8.6 mg, 0.019 mmol, 72.2% yield). ¹H NMR (500 MHz, methanol-d4) δ 9.67 (s, 1H), 8.50 (s, 1H), 8.20 (d, J=8.1 Hz, 1H), 8.14-8.07 (m, 2H), 7.83 (ddd, J=8.2, 6.1, 1.8 Hz, 1H), 6.03 (s, 1H), 3.12-2.95 (m, 2H), 2.68 (s, 3H), 1.49-1.35 (m, 4H), 1.24 (s, 9H), 0.85 (s, 6H) 2 protons on methylenes closest to piperidine nitrogen were not observed in HNMR. ESI-MS(+) m/z=463.2 (M+1).

### Example 268

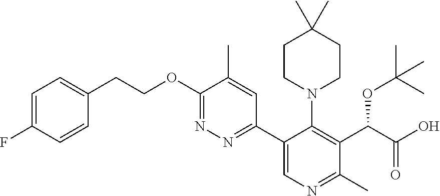 [SMILES: Cc1cc(-c2cnc(C)c([C@H](OC(C)(C)C)C(=O)O)c2N2CCC(C)(C)CC2)nnc1OCCc1ccc(F)cc1]

### (S)-2-(tert-Butoxy)-2-(4-(4, 4-dimethylpiperidin-1-yl)-5-(6-(4-fluorophenethoxy)-5-methylpyridazin-3-yl)-2-methylpyridin-3-yl)acetic acid

To a solution of isopropyl (S)-2-(tert-butoxy)-2-(4-(4,4-dimethylpiperidin-1-yl)-5-(6-(4-fluorophenethoxy)-5-methylpyridazin-3-yl)-2-methylpyridin-3-yl)acetate (56 mg, 0.092 mmol) in EtOH (1 mL) was added sodium hydroxide (0.185 mL, 0.923 mmol) then heated at 75° C. for 24 hrs. The reaction was cooled to RT, filtered through a nylon 0.45 g frit filter and purified via preparative LC/MS. Fractions containing the desired product were combined and dried via centrifugal evaporation to afford the product (S)-2-(tert-butoxy)-2-(4-(4,4-dimethylpiperidin-1-yl)-5-(6-(4-fluorophenethoxy)-5-methylpyridazin-3-yl)-2-methylpyridin-3-yl)acetic acid (36.5 mg, 0.065 mmol, 70.0% yield). ¹H NMR (500 MHz, METHANOL-d4) δ 8.07 (s, 1H), 7.39 (d, J=1.1 Hz, 1H), 7.25-7.18 (m, 2H), 7.01-6.89 (m, 2H), 5.87 (s, 1H), 4.41-4.29 (m, 2H), 3.20-3.04 (m, 4H), 2.87-2.73 (m, 2H), 2.63 (s, 3H), 2.26 (d, J=1.1 Hz, 3H), 1.54-1.39 (m, 4H), 1.21 (s, 9H), 0.94 (s, 6H). ESI-MS(+) m/z=565.3 (M+1).

### Example 269

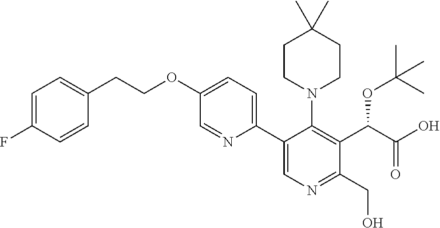 [SMILES: CC1(C)CCN(c2c(-c3ccc(OCCc4ccc(F)cc4)cn3)cnc(CO)c2[C@H](OC(C)(C)C)C(=O)O)CC1]

### (S)-2-(tert-Butoxy)-2-(4′-(4, 4-dimethylpiperidin-1-yl)-5-(4-fluorophenethoxy)-6′-(hydroxymethyl)-[2, 3′-bipyridin]-5′-yl)acetic acid

To a solution of (S)-5-(tert-butoxy)-4-(4,4-dimethylpiperidin-1-yl)-3-(5-(4-fluorophenethoxy)pyridin-2-yl)-5,8-dihydro-6H-pyrano[3,4-b]pyridin-6-one (17 mg, 0.031 mmol) in EtOH (1 mL) was added sodium hydroxide (0.062 mL, 0.310 mmol) then heated at 75° C. for 24 hrs. The reaction was cooled to RT, filtered through a nylon 0.45 t frit filter and purified via preparative LC/MS. Fractions containing the desired product were combined and dried via centrifugal evaporation to afford the product (S)-2-(tert-butoxy)-2-(4′-(4,4-dimethylpiperidin-1-yl)-5-(4-fluorophenethoxy)-6′-(hydroxymethyl)-[2,3′-bipyridin]-5′-yl)acetic acid (5.1 mg, 29% yield). ¹H NMR (500 MHz, methanol-d4) δ 8.35 (d, J=2.4 Hz, 1H), 8.22 (s, 1H), 7.57-7.51 (m, 1H), 7.50-7.44 (m, 1H), 7.40-7.33 (m, 2H), 7.06 (brt, J=8.7 Hz, 2H), 5.91 (d, J=13.7 Hz, 1H), 5.70 (s, 1H), 5.10-4.71 (m, 1H), 4.36 (br t, J=6.6 Hz, 2H), 3.15 (br t, J=6.4 Hz, 2H), 2.87-2.68 (m, 2H), 1.43 (br s, 4H), 1.20 (s, 9H), 0.93-0.87 (m, 6H) 2 protons (2 closest to N on piperidine) were not observed in the HNMR spectrum. ESI-MS(+) m/z=566.3 (M+1).

### Example 270

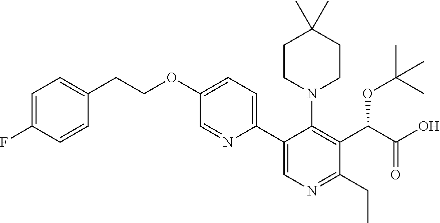 [SMILES: CCc1ncc(-c2ccc(OCCc3ccc(F)cc3)cn2)c(N2CCC(C)(C)CC2)c1[C@H](OC(C)(C)C)C(=O)O]

### Isopropyl (S)-2-(5-bromo-4-(4, 4-dimethylpiperidin-1-yl)-2-methylpyridin-3-yl)-2-isopropoxyacetate

To a solution of isopropyl (S)-2-(tert-butoxy)-2-(4′-(4,4-dimethylpiperidin-1-yl)-6′-ethyl-5-(4-fluorophenethoxy)-[2,3′-bipyridin]-5′-yl)acetate (63 mg, 0.104 mmol) in EtOH (2 mL) was added 5 N aq. sodium hydroxide (0.208 mL, 1.040 mmol) then heated at 75° C. for 24 hrs. The reaction was cooled to RT, filtered through a nylon 0.45 g frit filter and purified via prep HPLC. The desired fractions were collected and lyophilized to afford the product isopropyl (S)-2-(5-bromo-4-(4,4-dimethylpiperidin-1-yl)-2-methylpyridin-3-yl)-2-isopropoxyacetate (50.4 mg, 0.089 mmol, 86% yield) as a white fluffy solid. ¹H NMR (500 MHz, methanol-d4) δ 8.34 (d, J=2.8 Hz, 1H), 8.17 (s, 1H), 7.54 (dd, J=8.6, 2.9 Hz, 1H), 7.45 (d, J=8.7 Hz, 1H), 7.40-7.34 (m, 2H), 7.06 (t, J=8.7 Hz, 2H), 5.84 (s, 1H), 4.36 (td, J=6.7, 1.2 Hz, 2H), 3.15 (t, J=6.6 Hz, 2H), 3.04-2.67 (m, 4H), 1.43 (br s, 4H), 1.31 (t, J=7.4 Hz, 3H), 1.21 (s, 9H), 0.89 (s, 6H) 2 protons on methylenes closest to piperidine nitrogen were not observed in HNMR spectrum. ESI-MS(+) m/z=564.2 (M+1).

### Example 271

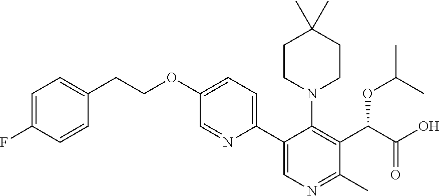 [SMILES: Cc1ncc(-c2ccc(OCCc3ccc(F)cc3)cn2)c(N2CCC(C)(C)CC2)c1[C@H](OC(C)C)C(=O)O]

### (S)-2-(4′-(4, 4-Dimethylpiperidin-1-yl)-5-(4-fluorophenethoxy)-6′-methyl-[2, 3′-bipyridin]-5′-yl)-2-isopropoxyacetic acid

To a solution of isopropyl (S)-2-(4′-(4,4-dimethylpiperidin-1-yl)-5-(4-fluorophenethoxy)-6′-methyl-[2,3′-bipyridin]-5′-yl)-2-isopropoxyacetate (15 mg, 0.026 mmol) in EtOH (1 mL) was added sodium hydroxide (0.052 mL, 0.260 mmol) then heated at 75° C. for 3 hrs. The reaction was cooled to RT, filtered through a nylon 0.45 t frit filter and purified via preparative LC/MS. Fractions containing the desired product were combined and dried via centrifugal evaporation to afford the product (S)-2-(4′-(4,4-dimethylpiperidin-1-yl)-5-(4-fluorophenethoxy)-6′-methyl-[2,3′-bipyridin]-5′-yl)-2-isopropoxyacetic acid (11 mg, 0.021 mmol, 79% yield). ¹H NMR (500 MHz, methanol-d₄) δ 8.33 (d, J=2.4 Hz, 1H), 8.10 (s, 1H), 7.53 (dd, J=8.5, 2.7 Hz, 1H), 7.44 (d, J=8.5 Hz, 1H), 7.37 (dd, J=8.1, 5.6 Hz, 2H), 7.06 (t, J=8.7 Hz, 2H), 5.67 (s, 1H), 4.35 (t, J=6.6 Hz, 2H), 3.49-3.41 (m, 1H), 3.15 (t, J=6.4 Hz, 2H), 2.76 (br s, 3H), 2.63 (s, 3H), 1.37 (br s, 4H), 1.26 (d, J=5.8 Hz, 3H), 1.02 (d, J=6.1 Hz, 3H), 0.87 (s, 6H). 1 proton (broadened peak of a methylene proton closest to piperine nitrogen) was not observed in the HNMR spectrum. ESI-MS(+) m/z=536.3 (M+1).

### Example 272

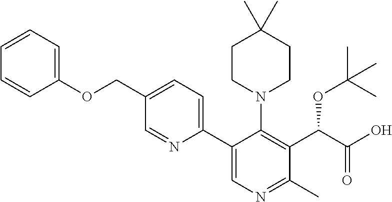 [SMILES: Cc1ncc(-c2ccc(COc3ccccc3)cn2)c(N2CCC(C)(C)CC2)c1[C@H](OC(C)(C)C)C(=O)O]

### (S)-2-(tert-Butoxy)-2-(4′-(4, 4-dimethylpiperidin-1-yl)-6′-methyl-5-(phenoxymethyl)-[2, 3′-bipyridin]-5′-yl)acetic acid

To a solution of isopropyl (S)-2-(tert-butoxy)-2-(4′-(4,4-dimethylpiperidin-1-yl)-6′-methyl-5-(phenoxymethyl)-[2,3′-bipyridin]-5′-yl)acetate (39 mg, 0.070 mmol) in EtOH (464 μl) was added sodium hydroxide (139 μl, 0.697 mmol) then heated at 75° C. for 24 hrs. The reaction was cooled to RT, filtered through a nylon 0.45μ frit filter and purified via preparative LC/MS. Fraction collection was triggered by MS and UV signals. Fractions containing the desired product were combined and dried via centrifugal evaporation to afford the product (S)-2-(tert-butoxy)-2-(4′-(4,4-dimethylpiperidin-1-yl)-6′-methyl-5-(phenoxymethyl)-[2,3′-bipyridin]-5′-yl)acetic acid (20 mg, 0.039 mmol, 55.5% yield). ¹H NMR (500 MHz, methanol-d4) δ 8.80 (s, 1H), 8.23 (s, 1H), 8.10 (br d, J=7.6 Hz, 1H), 7.60 (br d, J=7.9 Hz, 1H), 7.36-7.25 (m, 2H), 7.05 (br d, J=7.9 Hz, 1H), 6.98 (br t, J=7.2 Hz, 1H), 7.07-6.84 (m, 1H), 5.82 (s, 1H), 5.28 (s, 2H), 3.18-2.74 (m, 3H), 2.68 (s, 3H), 1.43 (br s, 4H), 1.21 (s, 9H), 0.88 (s, 6H). 1 proton on one of the methylenes closest to the piperidine N was not observed in the HNMR spectrum. ESI-MS(+) m/z=518.3 (M+1).

### Example 273

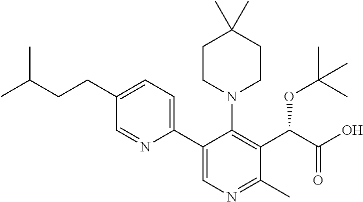 [SMILES: Cc1ncc(-c2ccc(CCC(C)C)cn2)c(N2CCC(C)(C)CC2)c1[C@H](OC(C)(C)C)C(=O)O]

### (S)-2-(tert-Butoxy)-2-(4′-(4, 4-dimethylpiperidin-1-yl)-5-isopentyl-6′-methyl-[2, 3′-bipyridin]-5′-yl)acetic acid

To an N₂ sparged solution (N₂ sparge for 5 minutes) of isopropyl (S,Z)-2-(tert-butoxy)-2-(4′-(4,4-dimethylpiperidin-1-yl)-6′-methyl-5-(3-methylbut-1-en-1-yl)-[2,3′-bipyridin]-5′-yl)acetate (164 mg, 0.314 mmol) in Ethanol (3 mL) was added Pd—C (33.5 mg, 0.031 mmol). The vessel was sealed and the solution bubbled with H₂ for 5 minutes. The reaction was then left under positive pressure of H₂ for 2 hrs. The reaction solution was sparged with N₂ and the solution filtered through a 0.45 nylon frit filter into a 7 mL vial. To the vial was added 5 N sodium hydroxide (0.629 mL, 3.14 mmol) and the solution stirred for 18 hrs at 75° C. The reaction was then cooled to rt and filtered through a 0.45μ nylon frit filter and purified via preparative LC/MS. Fractions containing the desired product were combined and dried via centrifugal evaporation to afford the product (S)-2-(tert-butoxy)-2-(4′-(4,4-dimethylpiperidin-1-yl)-5-isopentyl-6′-methyl-[2,3′-bipyridin]-5′-yl)acetic acid (27.2 mg, 18% yield). ¹H NMR (500 MHz, methanol-d4) δ 8.56 (s, 1H), 8.23 (s, 1H), 7.87 (br d, J=7.9 Hz, 1H), 7.50 (br d, J=7.9 Hz, 1H), 5.82 (s, 1H), 3.09-2.83 (m, 2H), 2.79 (br t, J=6.9 Hz, 2H), 2.68 (s, 3H), 1.61 (br s, 2H), 1.43 (br s, 4H), 1.36-1.29 (m, 1H), 1.22 (s, 9H), 1.00 (br d, J=4.0 Hz, 6H), 0.90 (s, 6H). 2 protons on the methylenes closest to piperidine N were not observed in the HNMR. ESI-MS(+) m/z=482.3 (M+1).

### Example 274

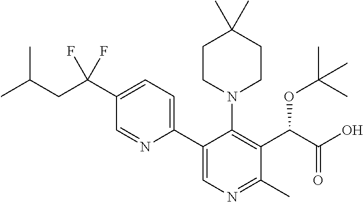 [SMILES: Cc1ncc(-c2ccc(C(F)(F)CC(C)C)cn2)c(N2CCC(C)(C)CC2)c1[C@H](OC(C)(C)C)C(=O)O]

### (S)-2-(tert-Butoxy)-2-(5-(1,1-difluoro-3-methylbutyl)-4′-(4, 4-dimethylpiperidin-1-yl)-6′-methyl-[2, 3′-bipyridin]-5′-yl)acetic acid

To a solution of isopropyl (S)-2-(tert-butoxy)-2-(5-(1,1-difluoro-3-methylbutyl)-4′-(4,4-dimethylpiperidin-1-yl)-6′-methyl-[2,3′-bipyridin]-5′-yl)acetate (152 mg, 0.272 mmol) in EtOH (1810 μl) was added sodium hydroxide (543 μl, 2.72 mmol) then heated at 75° C. for 24 hrs. The reaction was cooled to RT, filtered through a nylon 0.45 g frit filter and purified via preparative LC/MS. Fraction collection was triggered by MS and UV signals. Fractions containing the desired product were combined and dried via centrifugal evaporation to afford the product (S)-2-(tert-butoxy)-2-(5-(1,1-difluoro-3-methylbutyl)-4′-(4,4-dimethylpiperidin-1-yl)-6′-methyl-[2,3′-bipyridin]-5′-yl)acetic acid (24.8 mg, 0.046 mmol, 16.87% yield). ¹H NMR (500 MHz, methanol-d4) δ 8.86 (br s, 1H), 8.27 (br s, 1H), 8.15 (br d, J=7.6 Hz, 1H), 7.71 (br d, J=7.9 Hz, 1H), 5.80 (br s, 1H), 3.01 (br s, 1H), 2.84 (br s, 2H), 2.69 (br s, 3H), 2.22 (td, J=17.2, 6.3 Hz, 2H), 1.81 (dt, J=13.0, 6.4 Hz, 1H), 1.44 (br s, 3H), 1.22 (s, 9H), 1.00 (br s, 6H), 0.90 (s, 7H). 1 proton on one of the methylenes closest to the piperidine N was not observed in the HNMR spectrum. ESI-MS(+) m/z=518.3 (M+1).

### Example 275

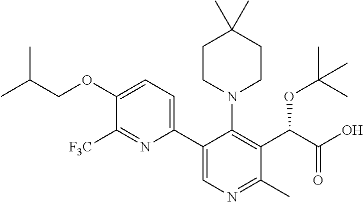 [SMILES: Cc1ncc(-c2ccc(OCC(C)C)c(C(F)(F)F)n2)c(N2CCC(C)(C)CC2)c1[C@H](OC(C)(C)C)C(=O)O]

### (S)-2-(tert-Butoxy)-2-(4′-(4, 4-dimethylpiperidin-1-yl)-5-isobutoxy-6′-methyl-6-(trifluoromethyl)-[2, 3′-bipyridin]-5′-yl)acetic acid

To a solution of isopropyl (S)-2-(tert-butoxy)-2-(4′-(4,4-dimethylpiperidin-1-yl)-5-isobutoxy-6′-methyl-6-(trifluoromethyl)-[2,3′-bipyridin]-5′-yl)acetate (99 mg, 0.167 mmol) in EtOH (1112 μl) was added sodium hydroxide (333 μl, 1.667 mmol) then heated at 75° C. for 24 hrs. The reaction was cooled to RT, filtered through a nylon 0.45 g frit filter and purified via preparative LC/MS. Fractions containing the desired product were combined and dried via centrifugal evaporation to afford the product (S)-2-(tert-butoxy)-2-(4′-(4,4-dimethylpiperidin-1-yl)-5-isobutoxy-6′-methyl-6-(trifluoromethyl)-[2,3′-bipyridin]-5′-yl)acetic acid (22.6 mg, 0.041 mmol, 24.57% yield). ¹H NMR (500 MHz, methanol-d4) δ 8.26 (s, 1H), 7.86-7.77 (m, 2H), 5.84 (br s, 1H), 4.02 (br d, J=6.1 Hz, 2H), 3.20-2.71 (m, 3H), 2.68 (s, 3H), 2.19 (dt, J=12.9, 6.5 Hz, 1H), 1.46 (br s, 4H), 1.22 (s, 9H), 1.11 (br d, J=6.4 Hz, 6H), 0.93 (s, 6H). 1 proton on one of the methylenes closest to piperidine N was not observed in the HNMR spectrum. ESI-MS(+) m/z=552.3 (M+1).

### Example 276

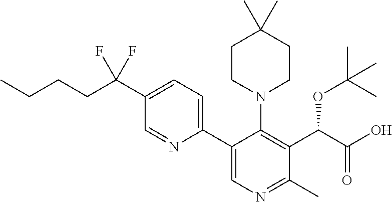 [SMILES: CCCCC(F)(F)c1ccc(-c2cnc(C)c([C@H](OC(C)(C)C)C(=O)O)c2N2CCC(C)(C)CC2)nc1]

### (S)-2-(tert-Butoxy)-2-(5-(1,1-difluoropentyl)-4′-(4, 4-dimethylpiperidin-1-yl)-6′-methyl-[2, 3′-bipyridin]-5′-yl)acetic acid

To a solution of isopropyl (S)-2-(tert-butoxy)-2-(5-(1,1-difluoropentyl)-4′-(4,4-dimethylpiperidin-1-yl)-6′-methyl-[2,3′-bipyridin]-5′-yl)acetate (130 mg, 0.232 mmol) in EtOH (1548 μl) was added aq. 5 N sodium hydroxide (464 μl, 2.322 mmol) then heated at 75° C. for 24 hrs. The reaction was cooled to RT, filtered through a nylon 0.45 g frit filter and purified via preparative LC/MS. Fraction collection was triggered by MS and UV signals. Fractions containing the desired product were combined and dried via centrifugal evaporation to afford the product (S)-2-(tert-butoxy)-2-(5-(1,1-difluoropentyl)-4′-(4,4-dimethylpiperidin-1-yl)-6′-methyl-[2,3′-bipyridin]-5′-yl)acetic acid (29.8 mg, 0.056 mmol, 24.12% yield). ¹H NMR (500 MHz, methanol-d4) δ 8.85 (s, 1H), 8.26 (br s, 1H), 8.14 (br d, J=7.9 Hz, 1H), 7.71 (br d, J=8.2 Hz, 1H), 5.79 (br s, 1H), 2.84 (br s, 3H), 2.69 (br s, 3H), 2.30 (br s, 2H), 1.47-1.37 (m, 8H), 1.21 (s, 9H), 0.96-0.86 (m, 10H). 1 proton on one of the methylenes closest to the piperidine N was not observed in the HNMR spectrum. ESI-MS(+) m/z=518.3 (M+1).

### Example 277

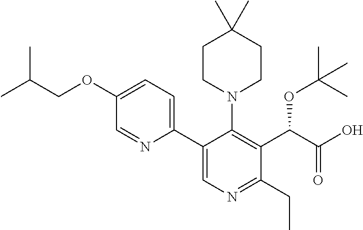 [SMILES: CCc1ncc(-c2ccc(OCC(C)C)cn2)c(N2CCC(C)(C)CC2)c1[C@H](OC(C)(C)C)C(=O)O]

### (S)-2-(tert-Butoxy)-2-(4′-(4, 4-dimethylpiperidin-1-yl)-6′-ethyl-5-isobutoxy-[2, 3′-bipyridin]-5′-yl)acetic acid

To a solution of isopropyl (S)-2-(tert-butoxy)-2-(4′-(4,4-dimethylpiperidin-1-yl)-6′-ethyl-5-isobutoxy-[2,3′-bipyridin]-5′-yl)acetate (16 mg, 0.030 mmol) in EtOH (1 mL) was added sodium hydroxide (0.059 mL, 0.296 mmol) then heated at 75° C. for 48 hrs. The reaction was cooled to RT, filtered through a nylon 0.45 g frit filter and purified via preparative LC/MS. Fractions containing the desired product were combined and dried via centrifugal evaporation to afford the product (S)-2-(tert-butoxy)-2-(4′-(4,4-dimethylpiperidin-1-yl)-6′-ethyl-5-isobutoxy-[2,3′-bipyridin]-5′-yl)acetic acid (5.4 mg, 10.85 μmol, 36.6% yield). ¹H NMR (500 MHz, methanol-d4) δ 8.38 (br s, 1H), 8.24 (s, 1H), 7.60-7.53 (m, 1H), 7.51-7.46 (m, 1H), 5.87 (br s, 1H), 3.93 (br d, J=6.4 Hz, 2H), 3.05-2.97 (m, 2H), 2.92-2.69 (m, 2H), 2.16 (dt, J=13.2, 6.7 Hz, 1H), 1.49-1.39 (m, 4H), 1.32 (br d, J=7.3 Hz, 3H), 1.23 (s, 9H), 1.10 (br d, J=6.4 Hz, 6H), 0.92 (s, 6H). 2 protons on the methylenes closest to the piperidine N were not observed in the HNMR spectrum. ESI-MS(+) m/z=498.3 (M+1).

### Example 278

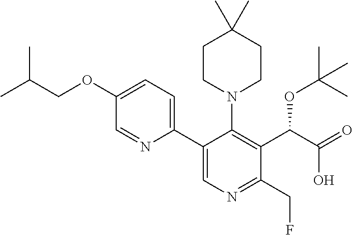 [SMILES: CC(C)COc1ccc(-c2cnc(CF)c([C@H](OC(C)(C)C)C(=O)O)c2N2CCC(C)(C)CC2)nc1]

### (S)-2-(tert-Butoxy)-2-(4′-(4, 4-dimethylpiperidin-1-yl)-6′-(fluoromethyl)-5-isobutoxy-[2, 3′-bipyridin]-5′-yl)acetic acid

To an N₂ sparged solution of benzyl (S)-2-(tert-butoxy)-2-(4′-(4,4-dimethylpiperidin-1-yl)-6′-(fluoromethyl)-5-isobutoxy-[2,3′-bipyridin]-5′-yl)acetate (23 mg, 0.039 mmol) in MeOH (2 mL) was added Pd—C (4.14 mg, 3.89 μmol) and capped with a rubber septum. H₂ was then bubbled through the solution for 10 minutes. The reaction was left under positive pressure of H₂ for 3 days. The LCMS indicated the reaction was complete. The reaction was filtered through a 0.45 t nylon frit filter and purified via preparative LC/MS. Fractions containing the desired product were combined and dried via centrifugal evaporation to afford the product (S)-2-(tert-butoxy)-2-(4′-(4,4-dimethylpiperidin-1-yl)-6′-(fluoromethyl)-5-isobutoxy-[2,3′-bipyridin]-5′-yl)acetic acid (9.3 mg, 0.018 mmol, 46.8% yield). ¹H NMR (500 MHz, methanol-d4) δ 8.37 (d, J=2.4 Hz, 1H), 8.31 (s, 1H), 7.60-7.53 (m, 1H), 7.52-7.43 (m, 1H), 5.87 (s, 1H), 5.74-5.55 (m, 2H), 3.93 (br d, J=6.4 Hz, 2H), 2.21-2.09 (m, 1H), 1.52-1.34 (m, 4H), 1.18 (s, 9H), 1.10 (d, J=6.7 Hz, 6H), 0.90 (s, 6H). 4 protons on the methylenes closest to the piperidine N were not observed in the HNMR spectrum. ESI-MS(+) m/z=502.3 (M+1).

### Example 279

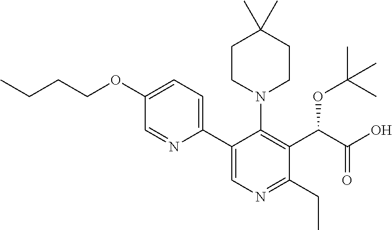 [SMILES: CCCCOc1ccc(-c2cnc(CC)c([C@H](OC(C)(C)C)C(=O)O)c2N2CCC(C)(C)CC2)nc1]

### (S)-2-(tert-Butoxy)-2-(5-butoxy-4′-(4, 4-dimethylpiperidin-1-yl)-6′-ethyl-[2, 3′-bipyridin]-5′-yl)acetic acid

To a 1 dram vial equipped with a stir bar and charged with isopropyl (S)-2-(tert-butoxy)-2-(4′-(4,4-dimethylpiperidin-1-yl)-6′-ethyl-5-hydroxy-[2,3′-bipyridin]-5′-yl)acetate (25 mg, 0.052 mmol) was added butan-1-ol (7.10 μl, 0.078 mmol). To the vial was added triphenylphosphane (20.34 mg, 0.078 mmol) as a solution in THF (0.25 mL). To the solution was added DIAD (0.015 mL, 0.078 mmol) as a solution in THF (0.25 mL). The solution was stirred at r.t. LCMS analysis at t=5 hr found clean and complete conversion to the desired ester intermediate. The reaction solution was concentrated under an N₂ stream. The residue was dissolved in EtOH (1.0 mL). To the solution was added NaOH (0.207 mL, 1.034 mmol). The solution was stirred at 75° C. for 18 hrs. LCMS analysis after 18 hrs found disappearance of the ester peak and a large expected product peak (acid). The mixture was filtered through a syringe filter, neutralized with acetic acid (0.059 mL, 1.034 mmol) and the filtrate was purified via preparative LC/MS. Fractions containing the desired product were combined and dried via centrifugal evaporation to afford the product (S)-2-(tert-butoxy)-2-(5-butoxy-4′-(4,4-dimethylpiperidin-1-yl)-6′-ethyl-[2,3′-bipyridin]-5′-yl)acetic acid (10.6 mg, 0.021 mmol, 41.2% yield). ¹H NMR (500 MHz, methanol-d4) δ 8.37 (br s, 1H), 8.24 (s, 1H), 7.60-7.53 (m, 1H), 7.52-7.46 (m, 1H), 5.85 (br s, 1H), 4.16 (br t, J=6.3 Hz, 2H), 3.00 (q, J=7.2 Hz, 2H), 2.93-2.70 (m, 2H), 1.90-1.79 (m, 2H), 1.57 (sxt, J=7.4 Hz, 2H), 1.45 (br s, 4H), 1.32 (br d, J=7.6 Hz, 3H), 1.23 (s, 9H), 1.03 (t, J=7.3 Hz, 3H), 0.91 (s, 6H). 2 protons on the methylenes closest to the piperidine N were not observed in the HNMR spectrum. ESI-MS(+) m/z=498.3 (M+1).

### Example 280

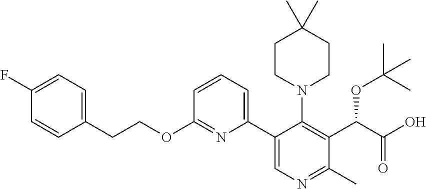 [SMILES: Cc1ncc(-c2cccc(OCCc3ccc(F)cc3)n2)c(N2CCC(C)(C)CC2)c1[C@H](OC(C)(C)C)C(=O)O]

### (S)-2-(tert-Butoxy)-2-(4′-(4, 4-dimethylpiperidin-1-yl)-6-(4-fluorophenethoxy)-6′-methyl-[2, 3′-bipyridin]-5′-yl)acetic acid

To a solution of isopropyl (S)-2-(tert-butoxy)-2-(4′-(4,4-dimethylpiperidin-1-yl)-6-(4-fluorophenethoxy)-6′-methyl-[2,3′-bipyridin]-5′-yl)acetate (132 mg, 0.223 mmol) in EtOH (1.5 mL) was added sodium hydroxide (0.446 mL, 2.231 mmol) then heated at 75° C. for -hrs. The reaction was cooled to RT, filtered through a nylon 0.45μ frit filter and purified via preparative LC/MS. Fractions containing the desired product were combined and dried via centrifugal evaporation to afford the product (S)-2-(tert-butoxy)-2-(4′-(4,4-dimethylpiperidin-1-yl)-6-(4-fluorophenethoxy)-6′-methyl-[2,3′-bipyridin]-5′-yl)acetic acid (20.5 mg, 17% yield). ¹H NMR (500 MHz, methanol-d4) δ 8.16 (s, 1H), 7.81 (br t, J=7.6 Hz, 1H), 7.28 (br t, J=6.6 Hz, 2H), 7.08 (br d, J=7.0 Hz, 1H), 6.99 (br t, J=8.5 Hz, 2H), 6.85 (br d, J=8.2 Hz, 1H), 5.78 (s, 1H), 4.63-4.44 (m, 2H), 3.23-3.09 (m, 1H), 3.06 (br t, J=6.4 Hz, 2H), 2.98-2.75 (m, 2H), 2.67 (s, 3H), 1.44 (br s, 4H), 1.21 (s, 9H), 0.88 (s, 6H). 1 proton on the methylenes closest to the piperidine N were not observed in the HNMR spectrum. ESI-MS(+) m/z=550.3 (M+1).

### Example 281 Example 282

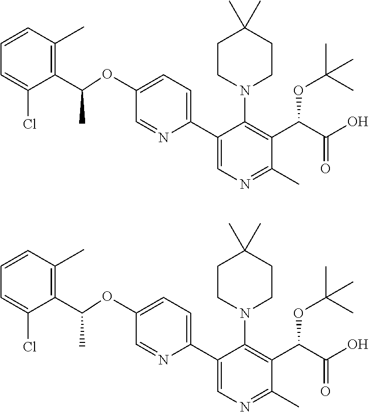

### (S)-2-(tert-Butoxy)-2-(5-((S)-1-(2-chloro-6-methylphenyl)ethoxy)-4′-(4, 4-dimethylpiperidin-1-yl)-6′-methyl-[2, 3′-bipyridin]-5′-yl)acetic acid and (S)-2-(tert-butoxy)-2-(5-((R)-1-(2-chloro-6-methylphenyl)ethoxy)-4′-(4, 4-dimethylpiperidin-1-yl)-6′-methyl-[2, 3′-bipyridin]-5′-yl)acetic acid

To a 1 dram vial equipped with a stir bar and charged with isopropyl (S)-2-(tert-butoxy)-2-(4′-(4,4-dimethylpiperidin-1-yl)-5-hydroxy-6′-methyl-[2,3′-bipyridin]-5′-yl)acetate (50 mg, 0.106 mmol) was added 1-(2-chloro-6-methylphenyl)ethan-1-ol (27.3 mg, 0.160 mmol). To the vial was added triphenylphosphane (41.9 mg, 0.160 mmol) as a solution in THF (0.25 mL). To the solution was added DIAD (0.031 mL, 0.160 mmol) as a solution in THF (0.25 mL). The solution was stirred at r.t. LCMS analysis at t=5 hr found clean and complete conversion to the desired ester intermediate. The reaction solution was concentrated under a N₂ stream. The residue was dissolved in EtOH (1.5 mL). To the solution was added NaOH (0.426 mL, 2.129 mmol). The solution was stirred at 75° C. for 18 hrs. The mixture was filtered through a syringe filter, neutralized with acetic acid (0.122 mL, 2.129 mmol) and the filtrate was purified via preparative LC/MS. Fractions containing the desired product were combined and dried via centrifugal evaporation to afford the products:

(First eluting): (S)-2-(tert-butoxy)-2-(5-((S)-1-(2-chloro-6-methylphenyl)ethoxy)-4′-(4,4-dimethylpiperidin-1-yl)-6′-methyl-[2,3′-bipyridin]-5′-yl)acetic acid (20.4 mg, 0.034 mmol, 31.7% yield): ¹H NMR (500 MHz, methanol-d4) δ 8.25 (s, 1H), 8.07 (s, 1H), 7.36 (s, 2H), 7.29 (br d, J=7.6 Hz, 1H), 7.17 (br t, J=7.6 Hz, 1H), 7.13-7.08 (m, 1H), 6.19 (br d, J=6.7 Hz, 1H), 5.73 (br s, 1H), 2.68 (br s, 2H), 2.62 (s, 3H), 2.52 (s, 3H), 1.80 (br d, J=6.4 Hz, 3H), 1.37 (br s, 4H), 1.16 (s, 9H), 0.88 (s, 6H). 2 protons on the methylenes closest to the piperidine N were not observed in the HNMR spectrum. ESI-MS(+) m/z=580.3 (M+1).

Second eluting: (S)-2-(tert-butoxy)-2-(5-((R)-1-(2-chloro-6-methylphenyl)ethoxy)-4′-(4,4-dimethylpiperidin-1-yl)-6′-methyl-[2,3′-bipyridin]-5′-yl)acetic acid (15.1 mg, 0.026 mmol, 24.45% yield): ¹H NMR (500 MHz, methanol-d₄) δ 8.28 (br s, 1H), 8.14 (s, 1H), 7.43-7.35 (m, 2H), 7.30 (br d, J=7.9 Hz, 1H), 7.18 (br t, J=7.8 Hz, 1H), 7.11 (br d, J=7.3 Hz, 1H), 6.20 (br d, J=6.7 Hz, 1H), 5.78 (br s, 1H), 3.09-2.67 (m, 3H), 2.64 (s, 3H), 2.50 (s, 3H), 1.80 (br d, J=6.4 Hz, 3H), 1.36 (br s, 4H), 1.19 (s, 9H), 0.78 (br s, 6H). 1 proton on the methylenes closest to the piperidine N were not observed in the HNMR spectrum. ESI-MS(+) m/z=580.3 (M+1).

### Example 283

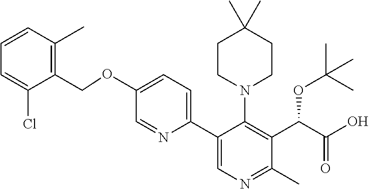 [SMILES: Cc1cccc(Cl)c1COc1ccc(-c2cnc(C)c([C@H](OC(C)(C)C)C(=O)O)c2N2CCC(C)(C)CC2)nc1]

### (S)-2-(tert-Butoxy)-2-(5-((2-chloro-6-methylbenzyl)oxy)-4′-(4,4-dimethylpiperidin-1-yl)-6′-methyl-[2, 3′-bipyridin]-5′-yl)acetic acid

To a 1 dram vial equipped with a stir bar and charged with isopropyl (S)-2-(tert-butoxy)-2-(4′-(4,4-dimethylpiperidin-1-yl)-5-hydroxy-6′-methyl-[2,3′-bipyridin]-5′-yl)acetate (25 mg, 0.053 mmol) was added (2-chloro-6-methylphenyl)methanol (12.51 mg, 0.080 mmol). To the vial was added triphenylphosphane (20.94 mg, 0.080 mmol) as a solution in THF (0.25 mL). To the solution was added DIAD (0.016 mL, 0.080 mmol) as a solution in THF (0.25 mL). The solution was stirred at r.t. LCMS analysis at t=5 hr found clean and complete conversion to the desired ester intermediate. The reaction solution was concentrated under a N₂ stream. The residue was dissolved in EtOH (1.0 mL). To the solution was added aq. 5 N NaOH (0.213 mL, 1.065 mmol). The solution was stirred at 75° C. for 18 hrs. LCMS analysis after 18 hrs found disappearance of the ester peak and a large expected product peak (acid). The mixture was filtered through a syringe filter, neutralized with acetic acid (0.061 mL, 1.065 mmol) and the filtrate was purified via preparative LC/MS. Fractions containing the desired product were combined and dried via centrifugal evaporation to afford the product (S)-2-(tert-butoxy)-2-(5-((2-chloro-6-methylbenzyl)oxy)-4′-(4,4-dimethylpiperidin-1-yl)-6′-methyl-[2,3′-bipyridin]-5′-yl)acetic acid (19 mg, 63% yield). ¹H NMR (500 MHz, methanol-d4) δ 8.47 (br s, 1H), 8.21 (s, 1H), 7.72 (br d, J=8.9 Hz, 1H), 7.53 (br d, J=8.5 Hz, 1H), 7.37-7.21 (m, 3H), 5.82 (br s, 1H), 5.43 (s, 2H), 3.16-2.70 (m, 3H), 2.67 (s, 3H), 2.50 (s, 3H), 1.45 (br s, 4H), 1.22 (s, 9H), 0.92 (s, 6H). 1 proton on the methylenes closest to the piperidine N were not observed in the HNMR spectrum. ESI-MS(+) m/z=566.3 (M+1).

### Example 284

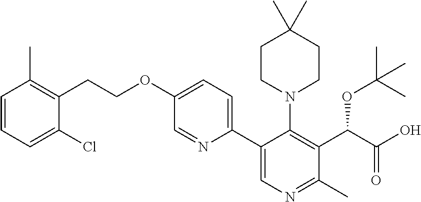 [SMILES: Cc1cccc(Cl)c1CCOc1ccc(-c2cnc(C)c([C@H](OC(C)(C)C)C(=O)O)c2N2CCC(C)(C)CC2)nc1]

### (S)-2-(tert-Butoxy)-2-(5-(2-chloro-6-methylphenethoxy)-4′-(4, 4-dimethylpiperidin-1-yl)-6′-methyl-[2, 3′-bipyridin]-5′-yl)acetic acid

To a 1 dram vial equipped with a stir bar and charged with isopropyl (S)-2-(tert-butoxy)-2-(4′-(4,4-dimethylpiperidin-1-yl)-5-hydroxy-6′-methyl-[2,3′-bipyridin]-5′-yl)acetate (25 mg, 0.053 mmol) was added 2-(2-chloro-6-methylphenyl)ethan-1-ol (13.63 mg, 0.080 mmol). To the vial was added triphenylphosphane (20.94 mg, 0.080 mmol) as a solution in THF (0.25 mL). To the solution was added DIAD (0.016 mL, 0.080 mmol) as a solution in THF (0.25 mL). The solution was stirred at r.t. LCMS analysis at t=5 hr found clean and complete conversion to the desired ester intermediate. The reaction solution was concentrated under a N₂ stream. The residue was dissolved in EtOH (1.0 mL). To the solution was added NaOH (0.213 mL, 1.065 mmol). The solution was stirred at 75° C. for 18 hrs. LCMS analysis after 18 hrs found disappearance of the ester peak and a large expected product peak (acid). The mixture was filtered through a syringe filter, neutralized with acetic acid (0.061 mL, 1.065 mmol) and the filtrate was purified via preparative LC/MS. Fractions containing the desired product were combined and dried via centrifugal evaporation to afford the product (S)-2-(tert-butoxy)-2-(5-(2-chloro-6-methylphenethoxy)-4′-(4,4-dimethylpiperidin-1-yl)-6′-methyl-[2,3′-bipyridin]-5′-yl)acetic acid (16 mg, 0.028 mmol, 51.8% yield). ¹H NMR (500 MHz, methanol-d4) δ 8.33 (br s, 1H), 8.08 (s, 1H), 7.53 (br d, J=8.5 Hz, 1H), 7.43 (br d, J=8.5 Hz, 1H), 7.27 (br d, J=7.3 Hz, 1H), 7.19-7.12 (m, 2H), 5.74 (br s, 1H), 4.36 (br t, J=6.9 Hz, 2H), 3.39 (br t, J=7.0 Hz, 2H), 2.90-2.68 (m, 2H), 2.64 (s, 3H), 2.49 (s, 3H), 1.50-1.37 (m, 4H), 1.18 (s, 9H), 0.89 (s, 6H). 2 protons on the methylenes closest to the piperidine N were not observed in the HNMR spectrum. ESI-MS(+) m/z=580.3 (M+1).

### Example 285

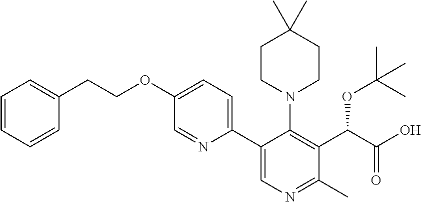 [SMILES: Cc1ncc(-c2ccc(OCCc3ccccc3)cn2)c(N2CCC(C)(C)CC2)c1[C@H](OC(C)(C)C)C(=O)O]

### (S)-2-(tert-Butoxy)-2-(4′-(4, 4-dimethylpiperidin-1-yl)-6′-methyl-5-phenethoxy-[2, 3′-bipyridin]-5′-yl)acetic acid

To a 1 dram vial equipped with a stir bar and charged with isopropyl (S)-2-(tert-butoxy)-2-(4′-(4,4-dimethylpiperidin-1-yl)-5-hydroxy-6′-methyl-[2,3′-bipyridin]-5′-yl)acetate (20 mg, 0.043 mmol) was added triphenylphosphane (16.76 mg, 0.064 mmol). To the vial was added 2-phenylethan-1-ol (7.80 mg, 0.064 mmol) as a solution in THF (0.25 mL). To the solution was added DIAD (0.012 mL, 0.064 mmol) as a solution in THF (0.25 mL). The solution was stirred at r.t. After 20 min. the reaction solution was concentrated under a N2 stream. The residue was dissolved in EtOH (1.0 mL). To the solution was added aq. NaOH (5.0 M, 0.100 mL, 0.500 mmol). The vial was placed in a 85 deg C. heating block with stirring for 5 h. The mixture was filtered through a 0.4 micron syringe filter and the filtrate was subjected to Prep-HPLC purification. Fractions containing the desired product were combined and dried via centrifugal evaporation to afford (S)-2-(tert-butoxy)-2-(4′-(4,4-dimethylpiperidin-1-yl)-6′-methyl-5-phenethoxy-[2,3′-bipyridin]-5′-yl)acetic acid (13.4 mg, 59% yield, 100% purity). ¹H NMR (500 MHz, METHANOL-d₄) δ 8.32 (d, J=2.7 Hz, 1H), 8.08 (s, 1H), 7.50 (dd, J=8.2, 2.7 Hz, 1H), 7.41 (d, J=8.5 Hz, 1H), 7.35-7.29 (m, 4H), 7.26-7.21 (m, 1H), 5.81 (s, 1H), 4.38 (br t, J=6.6 Hz, 2H), 3.15 (t, J=6.7 Hz, 2H), 3.02 (br s, 2H), 2.77 (br s, 2H), 2.64 (s, 3H), 1.41 (br s, 4H), 1.19 (s, 9H), 0.88 (s, 6H).

### Example 286

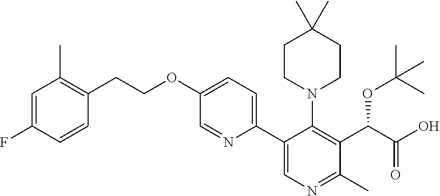 [SMILES: Cc1cc(F)ccc1CCOc1ccc(-c2cnc(C)c([C@H](OC(C)(C)C)C(=O)O)c2N2CCC(C)(C)CC2)nc1]

### (S)-2-(tert-Butoxy)-2-(4′-(4, 4-dimethylpiperidin-1-yl)-5-(4-fluoro-2-methylphenethoxy)-6′-methyl-[2, 3′-bipyridin]-5′-yl)acetic acid

To a 1 dram vial equipped with a stir bar and charged with isopropyl (S)-2-(tert-butoxy)-2-(4′-(4,4-dimethylpiperidin-1-yl)-5-hydroxy-6′-methyl-[2,3′-bipyridin]-5′-yl)acetate (20 mg, 0.043 mmol) was added triphenylphosphane (16.76 mg, 0.064 mmol). To the vial was added 2-(4-fluoro-2-methylphenyl)ethan-1-ol (9.85 mg, 0.064 mmol) as a solution in THF (0.25 mL). To the solution was added DIAD (0.012 mL, 0.064 mmol) as a solution in THF (0.25 mL). The solution was stirred at r.t. After 20 min the reaction solution was concentrated under a N₂ stream. The resulting residue was dissolved in ethanol (1.0 mL). To the solution was added aq. NaOH (5.0 M, 0.100 mL, 0.500 mmol). The vial was placed in a 85 deg C. heating block with stirring for 5 h. The mixture was filtered through a 0.4 micron syringe filter and the filtrate was subjected to Prep-HPLC purification. Fractions containing the desired product were combined and dried via centrifugal evaporation. The material was further purified via preparative LC/MS and fractions containing the desired product were combined and dried via centrifugal evaporation to afford (S)-2-(tert-butoxy)-2-(4′-(4,4-dimethylpiperidin-1-yl)-5-(4-fluoro-2-methylphenethoxy)-6′-methyl-[2,3′-bipyridin]-5′-yl)acetic acid (9.1 mg, 37% yield, 97.6% purity). ¹H NMR (500 MHz, METHANOL-d4) δ 8.40 (d, J=2.4 Hz, 1H), 8.28 (s, 1H), 7.56-7.49 (m, 2H), 7.27 (dd, J=8.2, 5.8 Hz, 1H), 6.95-6.91 (m, 1H), 6.87 (t, J=8.1 Hz, 1H), 5.74 (s, 1H), 4.36 (t, J=6.7 Hz, 2H), 3.16 (t, J=6.6 Hz, 2H), 3.00 (br s, 2H), 2.94 (br s, 2H), 2.78 (s, 3H), 2.41 (s, 3H), 1.52-1.42 (m, 4H), 1.24 (s, 9H), 0.95 (s, 6H). Alternative procedure: To a solution of (S)-isopropyl 2-(tert-butoxy)-2-(4′-(4,4-dimethylpiperidin-1-yl)-6′-methyl-5-phenethoxy-[2,3′-bipyridin]-5′-yl)acetate (210 mg, 0.366 mmol) in ethanol (3 mL) was added a solution of sodium hydroxide (146 mg, 3.66 mmol) dissolved in water (0.3 mL) and heated at 90° C. for 16 hr. Then, the pH was adjusted to about 7 with acetic acid, filtered and the filtrate was directly subjected to HPLC purification to afford (S)-2-(tert-butoxy)-2-(4′-(4,4-dimethylpiperidin-1-yl)-6′-methyl-5-phenethoxy-[2,3′-bipyridin]-5′-yl)acetic acid (66 mg, 0.121 mmol, 33.1% yield). LCMS (M+H)=532.

### Example 287

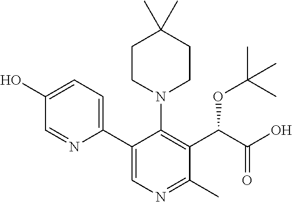 [SMILES: Cc1ncc(-c2ccc(O)cn2)c(N2CCC(C)(C)CC2)c1[C@H](OC(C)(C)C)C(=O)O]

### (S)-2-(tert-Butoxy)-2-(4′-(4, 4-dimethylpiperidin-1-yl)-5-hydroxy-6′-methyl-[2, 3′-bipyridin]-5′-yl)acetic acid

To a 1 dram vial charged with isopropyl (S)-2-(tert-butoxy)-2-(4′-(4,4-dimethylpiperidin-1-yl)-5-hydroxy-6′-methyl-[2,3′-bipyridin]-5′-yl)acetate (25 mg, 0.053 mmol) and equipped with a stir bar was added ethanol (1.0 mL), then aq. NaOH (0.106 mL, 0.532 mmol). The vial was placed in a 85 deg C. heating block with stirring for 18 h. The mixture was filtered through a 0.4 micron syringe filter and the filtrate was subjected to Prep-HPLC purification. Fractions containing the desired product were combined and dried via centrifugal evaporation to afford (S)-2-(tert-butoxy)-2-(4′-(4,4-dimethylpiperidin-1-yl)-5-hydroxy-6′-methyl-[2,3′-bipyridin]-5′-yl)acetic acid (17.7 mg, 74% yield, 95% purity). ¹H NMR (500 MHz, METHANOL-d4) δ 8.25 (s, 1H), 8.18-8.16 (m, 1H), 7.39 (s, 2H), 5.84 (br s, 1H), 3.03 (br s, 1H), 2.83 (br s, 2H), 2.68-2.64 (m, 1H), 2.66 (s, 3H), 2.66 (br s, 1H), 1.45 (br s, 4H), 1.21 (s, 9H), 0.92 (s, 6H).

### Example 288

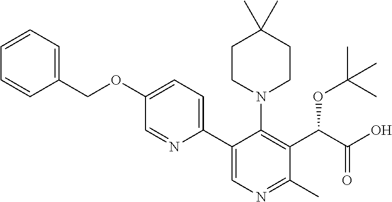 [SMILES: Cc1ncc(-c2ccc(OCc3ccccc3)cn2)c(N2CCC(C)(C)CC2)c1[C@H](OC(C)(C)C)C(=O)O]

### (S)-2-(5-(Benzyloxy)-4′-(4, 4-dimethylpiperidin-1-yl)-6′-methyl-[2, 3′-bipyridin]-5′-yl)-2-(tert-butoxy)acetic acid

To a 1 dram vial equipped with a stir bar was added isopropyl (S)-2-(5-(benzyloxy)-4′-(4,4-dimethylpiperidin-1-yl)-6′-methyl-[2,3′-bipyridin]-5′-yl)-2-(tert-butoxy)acetate (30 mg, 0.054 mmol), then ethanol (1.0 mL), then aq. sodium hydroxide (5.0 M, 0.11 mL, 0.54 mmol). The vial was capped, then placed in a 100 deg C. heating block with stirring for 2 h. The mixture was cooled to r.t and then filtered through a 0.4 micron syringe filter. The filtrate was subjected to Prep-HPLC purification. Fractions containing the desired product were combined and dried via centrifugal evaporation to afford (S)-2-(5-(benzyloxy)-4′-(4,4-dimethylpiperidin-1-yl)-6′-methyl-[2,3′-bipyridin]-5′-yl)-2-(tert-butoxy)acetic acid (18.3 mg, 65% yield, 99% purity). ¹H NMR (500 MHz, METHANOL-d4) δ 8.49 (d, J=2.7 Hz, 1H), 8.31 (s, 1H), 7.65 (dd, J=8.4, 2.9 Hz, 1H), 7.55-7.48 (m, 3H), 7.44-7.34 (m, 3H), 5.74 (s, 1H), 5.30 (s, 2H), 2.99 (br s, 2H), 2.90 (br s, 2H), 2.75 (s, 3H), 1.46 (br s, 4H), 1.23 (s, 9H), 0.93 (s, 6H).

Examples 289 to 417 were prepared by one of the general methods described below.

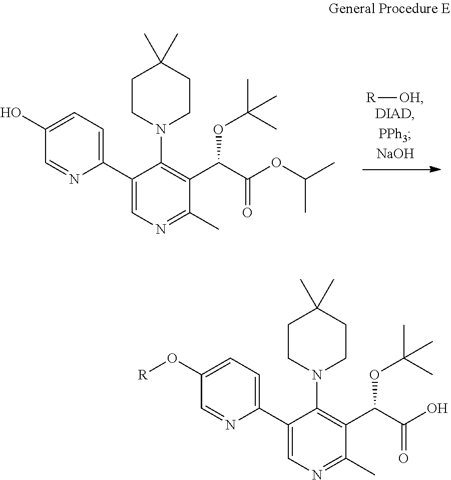

To a 1 dram vial equipped with a stir bar was added the alcohol (0.080 mmol), isopropyl (S)-2-(tert-butoxy)-2-(4′-(4,4-dimethylpiperidin-1-yl)-5-hydroxy-6′-methyl-[2,3′-bipyridin]-5′-yl)acetate (25 mg, 0.053 mmol), and triphenylphosphine (20.9 mg, 0.080 mmol) as a solution in THF (0.25 mL). To the solution was added DIAD (0.016 mL, 0.080 mmol) as a solution in THF (0.25 mL). The solution was stirred at r.t. for 30 min to 18 h. The reaction solution was concentrated under a N₂ stream. The residue was dissolved in EtOH (1.0 mL). To the solution was added aq. NaOH (5.0 M, 0.110 mL, 0.532 mmol). The vial was capped, then placed in a 85° C. heating block with stirring for 4 h to 18 h. The mixture was cooled to r.t., then was filtered through a 0.4 micron syringe filter and the filtrate was directly subjected to HPLC purification to afford the purified product.

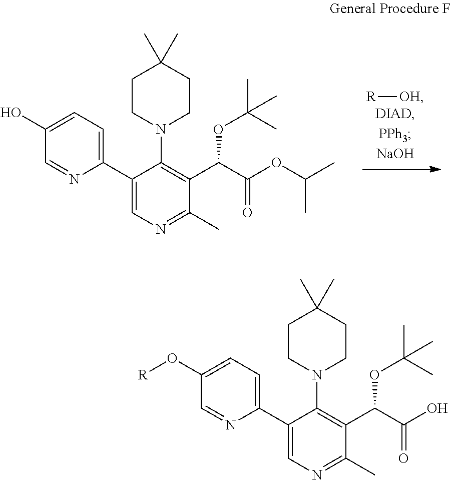

To a 1 dram vial equipped with a stir bar was added the alcohol (0.080 mmol), isopropyl (S)-2-(tert-butoxy)-2-(4′-(4,4-dimethylpiperidin-1-yl)-5-hydroxy-6′-methyl-[2,3′-bipyridin]-5′-yl)acetate as a solution in THF (0.355 M, 0.150 mL, 0.053 mmol), and triphenylphosphine as a solution in THF (0.535 M, 0.150 mL, 0.080 mmol). To the stirred solution was added diisopropyl diazene-1,2-dicarboxylate (“DIAD”) as a solution in THF (0.535 M, 0.150 mL, 0.080 mmol). The solution was stirred at r.t. for 30 min to 18 h. The reaction solution was concentrated under a N₂ stream. The residue was dissolved in EtOH (1.0 mL). To the solution was added aq. sodium hydroxide (5.0 M, 0.160 mL, 0.795 mmol). The vial was capped, then placed in a 85° C. heating block with stirring for 4 h to 18 h. The mixture was cooled to r.t. and to the mixture was added AcOH (0.050 mL). The mixture was filtered through a 0.4 micron syringe filter and the filtrate was directly subjected to HPLC purification to afford the purified product.

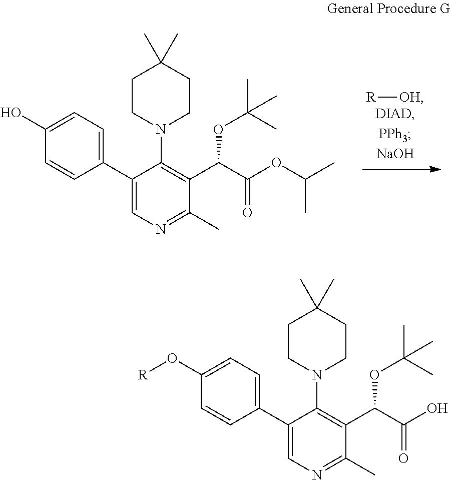

To a 1 dram vial equipped with a stir bar was added the alcohol (0.080 mmol), a solution of isopropyl (S)-2-(tert-butoxy)-2-(4-(4,4-dimethylpiperidin-1-yl)-5-(4-hydroxyphenyl)-2-methylpyridin-3-yl)acetate (0.355 M, 0.150 ml, 0.053 mmol) in THF, a solution of triphenylphosphane (0.535 M, 0.150 ml, 0.080 mmol) in THF, then a solution of diisopropyl (E)-diazene-1,2-dicarboxylate (0.535 M, 0.150 ml, 0.080 mmol) in THF. Alternately, to a 1 dram vial equipped with a stir bar was added the alcohol (0.080 mmol), then a solution of isopropyl (S)-2-(tert-butoxy)-2-(4-(4,4-dimethylpiperidin-1-yl)-5-(4-hydroxyphenyl)-2-methylpyridin-3-yl)acetate (0.355 M, 0.053 mmol), triphenylphosphane (0.535 M, 0.080 mmol) and diisopropyl (E)-diazene-1,2-dicarboxylate (0.535 M, 0.080 mmol) in THF (0.450 mL). In either preparation, the reaction solution was then stirred at r.t. for 18 h. The reaction solution was concentrated under a N₂ gas stream. To each vial was added EtOH (1.0 mL), then sodium hydroxide (aq.) (5.0 M, 0.160 ml, 0.795 mmol). The vial was capped, then placed in a 85° C. heating block for 4 h. The reaction mixture was cooled to r.t. and then filtered through a 0.4 micron syringe filter. The filtrate was subjected to HPLC purification to afford the purified product.

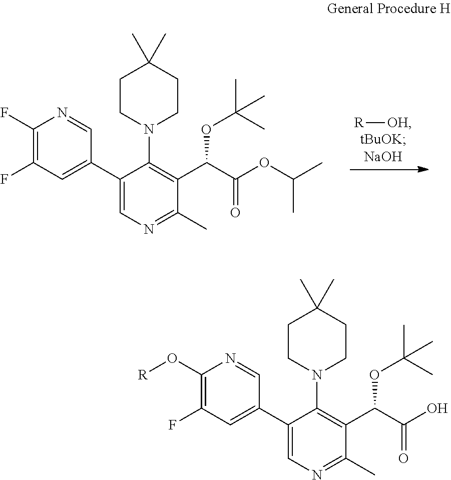

To a 1 dram vial equipped with a stir bar was added the alcohol (0.080 mmol), and isopropyl (S)-2-(tert-butoxy)-2-(4-(4,4-dimethylpiperidin-1-yl)-5′,6′-difluoro-6-methyl-[3,3′-bipyridin]-5-yl)acetate as a solution in THF (0.105 M, 0.500 ml, 0.053 mmol), then potassium tert-butoxide in THF (1.00 M, 0.077 mL, 0.077 mmol). The solution was stirred at r.t. for 15 min, then the volatiles were evaporated under a N₂ gas stream. The residue was dissolved in EtOH (1.0 mL). To the solution was added aq. sodium hydroxide (5.0 M, 0.160 ml, 0.795 mmol). The vial was capped, then placed in a 85° C. heating block with stirring for 1 h. The mixture was cooled to r.t. To the mixture was added acetic acid (0.050 mL). The mixture was filtered through a 0.4 micron syringe filter. The filtrate directly subjected to HPLC purification to afford the purified product.

### Example 289

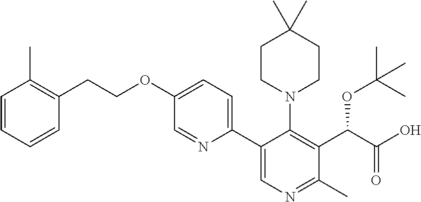 [SMILES: Cc1ccccc1CCOc1ccc(-c2cnc(C)c([C@H](OC(C)(C)C)C(=O)O)c2N2CCC(C)(C)CC2)nc1]

General Procedure E was followed. The experiment afforded the desired compound, (2S)-2-(tert-butoxy)-2-[4′-(4,4-dimethylpiperidin-1-yl)-6′-methyl-5-[2-(2-methylphenyl)ethoxy]-[2,3′-bipyridine]-5′-yl]acetic acid (8.7 mg, 30% yield, 98.8% purity). LCMS observed ion=546.2. ¹H NMR (500 MHz, METHANOL-d4) δ 8.42 (d, J=2.7 Hz, 1H), 8.34 (s, 1H), 7.59-7.52 (m, 2H), 7.27 (d, J=6.3 Hz, 1H), 7.20-7.13 (m, 3H), 5.71 (s, 1H), 4.37 (t, J=6.9 Hz, 2H), 3.20 (t, J=6.9 Hz, 2H), 2.96 (br s, 4H), 2.79 (s, 3H), 2.41 (s, 3H), 1.56-1.44 (m, 4H), 1.24 (s, 9H), 0.96 (s, 6H).

### Example 290

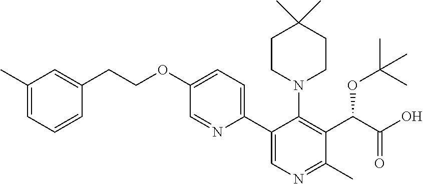 [SMILES: Cc1cccc(CCOc2ccc(-c3cnc(C)c([C@H](OC(C)(C)C)C(=O)O)c3N3CCC(C)(C)CC3)nc2)c1]

General Procedure E was followed. The experiment afforded the desired compound, (2S)-2-(tert-butoxy)-2-[4′-(4,4-dimethylpiperidin-1-yl)-6′-methyl-5-[2-(3-methylphenyl)ethoxy]-[2,3′-bipyridine]-5′-yl]acetic acid (11.4 mg, 39% yield, 98.5% purity). LCMS observed ion=546.3. ¹H NMR (500 MHz, METHANOL-d4) δ 8.42 (d, J=2.7 Hz, 1H), 8.35 (s, 1H), 7.59-7.53 (m, 2H), 7.22-7.19 (m, 1H), 7.17 (s, 1H), 7.13 (d, J=7.3 Hz, 1H), 7.07 (d, J=7.6 Hz, 1H), 5.71 (s, 1H), 4.37 (t, J=6.9 Hz, 2H), 3.12 (t, J=6.7 Hz, 2H), 3.08-2.88 (m, 4H), 2.79 (s, 3H), 2.35 (s, 3H), 1.54-1.44 (m, 4H), 1.24 (s, 9H), 0.96 (s, 6H).

Alternative Procedure:

To a solution of (S)-isopropyl 2-(tert-butoxy)-2-(4′-(4,4-dimethylpiperidin-1-yl)-6′-methyl-5-(3-methylphenethoxy)-[2,3′-bipyridin]-5′-yl)acetate (280 mg, 0.476 mmol) in methanol (8 mL) was added sodium hydroxide (381 mg, 9.53 mmol) in water (3 mL) and stirred at 80° C. overnight. Then, the mixture was acidified with aq. HCl to pH=7 and concentrated The crude was purified by Prep-HPLC to afford (S)-2-(tert-butoxy)-2-(4′-(4,4-dimethylpiperidin-1-yl)-6′-methyl-5-(3-methylphenethoxy)-[2,3′-bipyridin]-5′-yl)acetic acid (210 mg, 0.381 mmol, 80% yield) as a white solid. LCMS [M+H] 546.

### Example 291

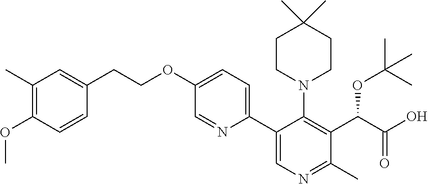 [SMILES: COc1ccc(CCOc2ccc(-c3cnc(C)c([C@H](OC(C)(C)C)C(=O)O)c3N3CCC(C)(C)CC3)nc2)cc1C]

General Procedure E was followed. The experiment afforded the desired compound, (2S)-2-(tert-butoxy)-2-[4′-(4,4-dimethylpiperidin-1-yl)-5-[2-(4-methoxy-3-methylphenyl)ethoxy]-6′-methyl-[2,3′-bipyridine]-5′-yl]acetic acid (11.1 mg, 36% yield, 100% purity). LCMS observed ion=576.4. 1H NMR (500 MHz, METHANOL-d4) δ 8.42 (s, 1H), 8.36 (d, J=1.5 Hz, 1H), 7.56 (q, J=9.0 Hz, 2H), 7.14-7.09 (m, 2H), 6.85 (d, J=8.2 Hz, 1H), 5.71 (s, 1H), 4.35-4.31 (m, 2H), 3.82 (d, J=1.8 Hz, 3H), 3.06 (br t, J=6.7 Hz, 2H), 2.96 (br s, 4H), 2.79 (d, J=1.5 Hz, 3H), 2.19 (s, 3H), 1.56-1.43 (m, 4H), 1.25 (d, J=1.5 Hz, 9H), 0.98-0.94 (m, 6H).

### Example 292

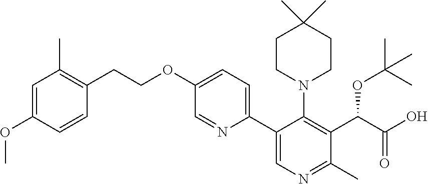 [SMILES: COc1ccc(CCOc2ccc(-c3cnc(C)c([C@H](OC(C)(C)C)C(=O)O)c3N3CCC(C)(C)CC3)nc2)c(C)c1]

General Procedure E was followed. The experiment afforded the desired compound, (2S)-2-(tert-butoxy)-2-[4′-(4,4-dimethylpiperidin-1-yl)-5-[2-(4-methoxy-2-methylphenyl)ethoxy]-6′-methyl-[2,3′-bipyridine]-5′-yl]acetic acid (12.2 mg, 40% yield, 100% purity). LCMS observed ion=576.3. ¹H NMR (500 MHz, METHANOL-d4) δ 8.32 (d, J=2.7 Hz, 1H), 8.08 (s, 1H), 7.52 (dd, J=8.5, 2.7 Hz, 1H), 7.42 (d, J=8.5 Hz, 1H), 7.18 (d, J=8.5 Hz, 1H), 6.77 (s, 1H), 6.73 (d, J=8.1 Hz, 1H), 5.76 (s, 1H), 4.30 (t, J=7.0 Hz, 2H), 3.77 (s, 3H), 3.12 (t, J=6.9 Hz, 2H), 2.74 (br s, 2H), 2.64 (s, 3H), 2.38 (s, 3H), 1.42 (br s, 4H), 1.18 (s, 9H), 0.89 (s, 6H).

### Example 293

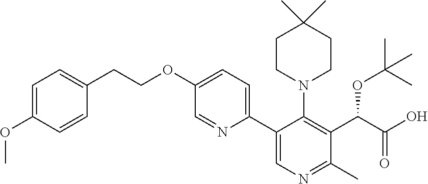 [SMILES: COc1ccc(CCOc2ccc(-c3cnc(C)c([C@H](OC(C)(C)C)C(=O)O)c3N3CCC(C)(C)CC3)nc2)cc1]

General Procedure E was followed. The experiment afforded the desired compound, (2S)-2-(tert-butoxy)-2-[4′-(4,4-dimethylpiperidin-1-yl)-5-[2-(4-methoxyphenyl)ethoxy]-6′-methyl-[2,3′-bipyridine]-5′-yl]acetic acid (16.1 mg, 54% yield, 100% purity). LCMS observed ion=562.3. ¹H NMR (500 MHz, METHANOL-d4) δ 8.32 (d, J=3.1 Hz, 1H), 8.09 (s, 1H), 7.52 (dd, J=8.5, 2.7 Hz, 1H), 7.43 (d, J=8.5 Hz, 1H), 7.27-7.24 (m, J=8.5 Hz, 2H), 6.91-6.87 (m, J=8.5 Hz, 2H), 5.76 (s, 1H), 4.32 (t, J=6.7 Hz, 2H), 3.79 (s, 3H), 3.09 (t, J=6.7 Hz, 2H), 2.74 (br s, 2H), 2.64 (s, 3H), 1.42 (br s, 4H), 1.19 (s, 9H), 0.89 (s, 6H).

Alternative Procedure:

To a solution of (S)-isopropyl 2-(tert-butoxy)-2-(4′-(4,4-dimethylpiperidin-1-yl)-5-(4-methoxyphenethoxy)-6′-methyl-[2,3′-bipyridin]-5′-yl)acetate (0.290 g, 0.480 mmol) in ethanol (5 mL) and water (1.0 mL)) was added NaoH (0.192 g, 4.80 mmol) at once. The resulting mixture was stirred at 90° C. for 10 hours. Then, the reaction was cooled, diluted with EtOAc (10 ml), washed with 1 N HCl and brine (10 ml each), dried by Na2SO4, filtered and concentrated to give crude product. The crude product was purified by prep-HPLC to give the desired product (S)-2-(tert-butoxy)-2-(4′-(4,4-dimethylpiperidin-1-yl)-5-(4-methoxyphenethoxy)-6′-methyl-[2,3′-bipyridin]-5′-yl)acetic acid (140 mg, 0.243 mmol, 50.6% yield) as white solid. LCMS (M+H)=562.3.

### Example 294

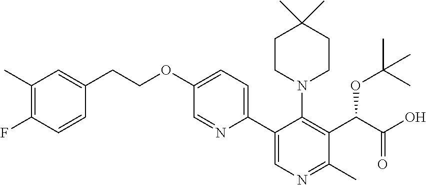 [SMILES: Cc1cc(CCOc2ccc(-c3cnc(C)c([C@H](OC(C)(C)C)C(=O)O)c3N3CCC(C)(C)CC3)nc2)ccc1F]

General Procedure E was followed. The experiment afforded the desired compound, (2S)-2-(tert-butoxy)-2-[4′-(4,4-dimethylpiperidin-1-yl)-5-[2-(4-fluoro-3-methylphenyl)ethoxy]-6′-methyl-[2,3′-bipyridine]-5′-yl]acetic acid (10.4 mg, 34% yield, 98.5% purity). LCMS observed ion=564.2. ¹H NMR (500 MHz, METHANOL-d4) δ 8.42 (d, J=2.4 Hz, 1H), 8.35 (s, 1H), 7.59-7.53 (m, 2H), 7.22 (br d, J=7.0 Hz, 1H), 7.18-7.14 (m, 1H), 6.97 (t, J=8.7 Hz, 1H), 5.70 (s, 1H), 4.36 (t, J=6.6 Hz, 2H), 3.11 (t, J=6.6 Hz, 2H), 2.96 (br s, 4H), 2.79 (s, 3H), 2.27 (s, 3H), 1.54-1.42 (m, 4H), 1.24 (s, 9H), 0.96 (s, 6H).

### Example 295

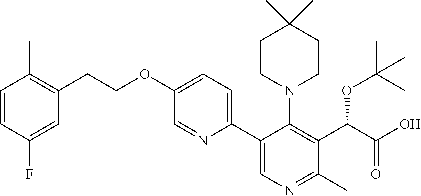 [SMILES: Cc1ccc(F)cc1CCOc1ccc(-c2cnc(C)c([C@H](OC(C)(C)C)C(=O)O)c2N2CCC(C)(C)CC2)nc1]

General Procedure E was followed. The experiment afforded the desired compound, (2S)-2-(tert-butoxy)-2-[4′-(4,4-dimethylpiperidin-1-yl)-5-[2-(5-fluoro-2-methylphenyl)ethoxy]-6′-methyl-[2,3′-bipyridine]-5′-yl]acetic acid (9.7 mg, 32% yield, 97.6% purity). LCMS observed ion=564.2. ¹H NMR (500 MHz, METHANOL-d4) δ 8.33 (d, J=3.1 Hz, 1H), 8.08 (s, 1H), 7.54 (dd, J=8.5, 2.7 Hz, 1H), 7.43 (d, J=8.5 Hz, 1H), 7.19 (t, J=7.2 Hz, 1H), 7.05 (dd, J=9.9, 2.6 Hz, 1H), 6.88 (td, J=8.5, 2.7 Hz, 1H), 5.75 (s, 1H), 4.37 (t, J=6.7 Hz, 2H), 3.18 (t, J=6.7 Hz, 2H), 2.74 (br s, 2H), 2.64 (s, 3H), 2.38 (s, 3H), 1.42 (br s, 4H), 1.18 (s, 9H), 0.89 (s, 6H).

### Example 296

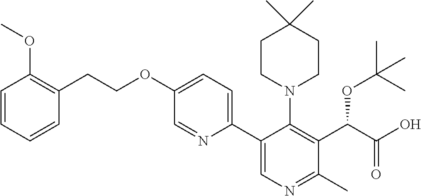 [SMILES: COc1ccccc1CCOc1ccc(-c2cnc(C)c([C@H](OC(C)(C)C)C(=O)O)c2N2CCC(C)(C)CC2)nc1]

General Procedure E was followed. The experiment afforded the desired compound, (2S)-2-(tert-butoxy)-2-[4′-(4,4-dimethylpiperidin-1-yl)-5-[2-(2-methoxyphenyl)ethoxy]-6′-methyl-[2,3′-bipyridine]-5′-yl]acetic acid (10 mg, 33% yield, 99% purity). LCMS observed ion=562.2. ¹H NMR (500 MHz, METHANOL-d4) δ 8.43 (d, J=2.7 Hz, 1H), 8.36 (s, 1H), 7.59-7.53 (m, 2H), 7.27-7.23 (m, 2H), 6.99 (d, J=8.5 Hz, 1H), 6.91 (t, J=7.3 Hz, 1H), 5.71 (s, 1H), 4.35 (t, J=7.2 Hz, 2H), 3.89 (s, 3H), 3.16 (t, J=7.2 Hz, 2H), 2.97 (br s, 4H), 2.79 (s, 3H), 1.55-1.44 (m, 4H), 1.24 (s, 9H), 0.97 (s, 6H).

### Example 297

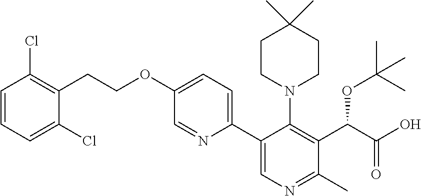 [SMILES: Cc1ncc(-c2ccc(OCCc3c(Cl)cccc3Cl)cn2)c(N2CCC(C)(C)CC2)c1[C@H](OC(C)(C)C)C(=O)O]

General Procedure E was followed. The experiment afforded the desired compound, (2S)-2-(tert-butoxy)-2-{5-[2-(2,6-dichlorophenyl)ethoxy]-4′-(4,4-dimethylpiperidin-1-yl)-6′-methyl-[2,3′-bipyridine]-5′-yl}acetic acid (10.9 mg, 34% yield, 100% purity). LCMS observed ion=600.2. ¹H NMR (500 MHz, METHANOL-d4) δ 8.43 (d, J=2.4 Hz, 1H), 8.35 (s, 1H), 7.61 (dd, J=8.5, 2.4 Hz, 1H), 7.55 (d, J=8.9 Hz, 1H), 7.43 (d, J=7.9 Hz, 2H), 7.27 (t, J=8.1 Hz, 1H), 5.70 (s, 1H), 4.44 (t, J=7.0 Hz, 2H), 3.59-3.52 (m, 2H), 2.96 (br s, 4H), 2.79 (s, 3H), 1.49 (br d, J=4.0 Hz, 4H), 1.24 (s, 9H), 0.97 (s, 6H).

Alternative Procedure:

To a solution of (S)-isopropyl 2-(tert-butoxy)-2-(5-(2,6-dichlorophenethoxy)-4′-(4,4-dimethylpiperidin-1-yl)-6′-methyl-[2,3′-bipyridin]-5′-yl)acetate (300 mg, 0.467 mmol) in methanol (10 mL) and water (2.00 mL) was added sodium hydroxide (373 mg, 9.34 mmol) and stirred for 20 hours at 100° C. Then, neutralized and purified by Prep-HPLC to give desired product (S)-2-(tert-butoxy)-2-(5-(2,6-dichlorophenethoxy)-4′-(4,4-dimethylpiperidin-1-yl)-6′-methyl-[2,3′-bipyridin]-5′-yl)acetic acid (110 mg, 0.180 mmol, 38.6% yield) as white solid. LCMS (M+H)=600.2.

### Example 298

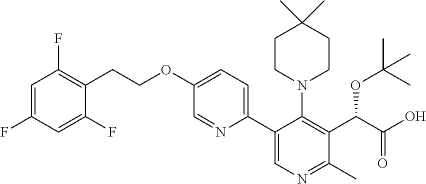 [SMILES: Cc1ncc(-c2ccc(OCCc3c(F)cc(F)cc3F)cn2)c(N2CCC(C)(C)CC2)c1[C@H](OC(C)(C)C)C(=O)O]

General Procedure E was followed. The experiment afforded the desired compound, (2S)-2-(tert-butoxy)-2-[4′-(4,4-dimethylpiperidin-1-yl)-6′-methyl-5-[2-(2,4,6-trifluorophenyl)ethoxy]-[2,3′-bipyridine]-5′-yl]acetic acid (10.5 mg, 33% yield, 97.6% purity). LCMS observed ion=586.2. ¹H NMR (500 MHz, METHANOL-d4) δ 8.40 (s, 1H), 7.59-7.52 (m, 2H), 6.89 (t, J=8.4 Hz, 2H), 5.58 (br s, 1H), 4.37 (t, J=6.4 Hz, 2H), 3.21 (t, J=6.4 Hz, 2H), 2.92 (br s, 4H), 2.78 (br s, 3H), 1.47 (br s, 4H), 1.23 (s, 9H), 0.95 (s, 6H).

### Example 299

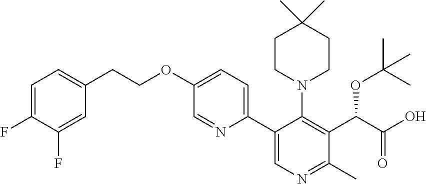 [SMILES: Cc1ncc(-c2ccc(OCCc3ccc(F)c(F)c3)cn2)c(N2CCC(C)(C)CC2)c1[C@H](OC(C)(C)C)C(=O)O]

General Procedure E was followed. The experiment afforded the desired compound, (2S)-2-(tert-butoxy)-2-{5-[2-(3,4-difluorophenyl)ethoxy]-4′-(4,4-dimethylpiperidin-1-yl)-6′-methyl-[2,3′-bipyridine]-5′-yl}acetic acid (11.3 mg, 37% yield, 97% purity). LCMS observed ion=568.2. ¹H NMR (500 MHz, METHANOL-d4) δ 8.42 (br s, 1H), 8.35 (br s, 1H), 7.60-7.53 (m, 2H), 7.29 (t, J=9.5 Hz, 1H), 7.24-7.18 (m, 1H), 7.18-7.13 (m, 1H), 5.67 (br s, 1H), 4.38 (t, J=6.4 Hz, 2H), 3.16 (t, J=6.3 Hz, 2H), 2.95 (br s, 4H), 2.78 (s, 3H), 1.48 (br s, 4H), 1.24 (s, 9H), 0.95 (s, 6H).

### Example 300

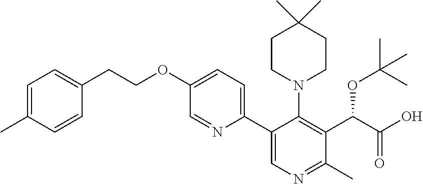 [SMILES: Cc1ccc(CCOc2ccc(-c3cnc(C)c([C@H](OC(C)(C)C)C(=O)O)c3N3CCC(C)(C)CC3)nc2)cc1]

General Procedure E was followed. The experiment afforded the desired compound, (2S)-2-(tert-butoxy)-2-[4′-(4,4-dimethylpiperidin-1-yl)-6′-methyl-5-[2-(4-methylphenyl)ethoxy]-[2,3′-bipyridine]-5′-yl]acetic acid (13.3 mg, 45% yield, 98% purity). LCMS observed ion=546.2. ¹H NMR (500 MHz, METHANOL-d4) δ 8.41 (d, J=2.7 Hz, 1H), 8.35 (s, 1H), 7.59-7.52 (m, 2H), 7.24-7.21 (m, J=7.9 Hz, 2H), 7.16-7.13 (m, J=7.9 Hz, 2H), 5.71 (s, 1H), 4.36 (t, J=6.7 Hz, 2H), 3.12 (t, J=6.7 Hz, 2H), 2.95 (br s, 4H), 2.79 (s, 3H), 2.33 (s, 3H), 1.53-1.44 (m, 4H), 1.24 (s, 9H), 0.98-0.92 (m, 6H).

### Example 301

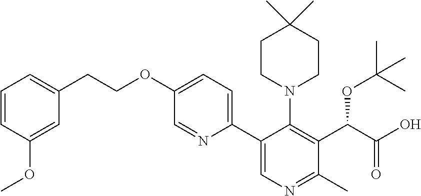 [SMILES: COc1cccc(CCOc2ccc(-c3cnc(C)c([C@H](OC(C)(C)C)C(=O)O)c3N3CCC(C)(C)CC3)nc2)c1]

General Procedure E was followed. The experiment afforded the desired compound, (2S)-2-(tert-butoxy)-2-[4′-(4,4-dimethylpiperidin-1-yl)-5-[2-(3-methoxyphenyl)ethoxy]-6′-methyl-[2,3′-bipyridine]-5′-yl]acetic acid (11.7 mg, 39% yield, 98% purity). LCMS observed ion=562.2. ¹H NMR (500 MHz, METHANOL-d4) δ 8.42 (d, J=2.7 Hz, 1H), 8.35 (s, 1H), 7.59-7.52 (m, 2H), 7.24 (t, J=8.1 Hz, 1H), 6.92 (s, 1H), 6.92 (d, J=6.7 Hz, 1H), 6.82 (d, J=8.2 Hz, 1H), 5.71 (s, 1H), 4.39 (t, J=6.7 Hz, 2H), 3.81 (s, 3H), 3.14 (t, J=6.6 Hz, 2H), 2.99 (br s, 2H), 2.95 (br s, 2H), 2.78 (s, 3H), 1.54-1.43 (m, 4H), 1.24 (s, 9H), 0.96 (s, 6H).

### Example 302

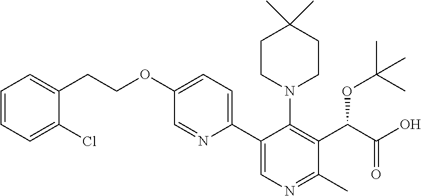 [SMILES: Cc1ncc(-c2ccc(OCCc3ccccc3Cl)cn2)c(N2CCC(C)(C)CC2)c1[C@H](OC(C)(C)C)C(=O)O]

General Procedure F was followed. The experiment afforded the desired compound, (2S)-2-(tert-butoxy)-2-{5-[2-(2-chlorophenyl)ethoxy]-4′-(4,4-dimethylpiperidin-1-yl)-6′-methyl-[2,3′-bipyridine]-5′-yl}acetic acid (22.4 mg, 74% yield, 98.6% purity). LCMS observed ion=566.2. ¹H NMR (500 MHz, METHANOL-d4) δ 8.43 (d, J=2.7 Hz, 1H), 8.35 (s, 1H), 7.60-7.53 (m, 2H), 7.47-7.41 (m, 2H), 7.32-7.25 (m, 2H), 5.70 (s, 1H), 4.43 (t, J=6.9 Hz, 2H), 3.35-3.31 (m, 2H), 2.95 (br s, 4H), 2.79 (s, 3H), 1.54-1.43 (m, 4H), 1.24 (s, 9H), 0.96 (s, 6H).

### Example 303

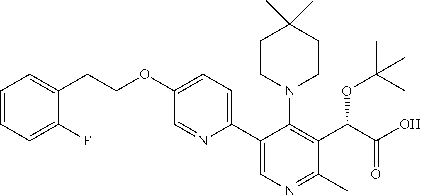 [SMILES: Cc1ncc(-c2ccc(OCCc3ccccc3F)cn2)c(N2CCC(C)(C)CC2)c1[C@H](OC(C)(C)C)C(=O)O]

General Procedure F was followed. The experiment afforded the desired compound, (2S)-2-(tert-butoxy)-2-[4′-(4,4-dimethylpiperidin-1-yl)-5-[2-(2-fluorophenyl)ethoxy]-6′-methyl-[2,3′-bipyridine]-5′-yl]acetic acid (20 mg, 68% yield, 100% purity). LCMS observed ion=550.3. ¹H NMR (500 MHz, METHANOL-d4) δ 8.42 (d, J=2.4 Hz, 1H), 8.35 (s, 1H), 7.58 (d, J=8.3 Hz, 1H), 7.54 (d, J=8.5 Hz, 1H), 7.41 (t, J=7.6 Hz, 1H), 7.32-7.27 (m, 1H), 7.16 (t, J=7.4 Hz, 1H), 7.10 (t, J=9.3 Hz, 1H), 5.71 (s, 1H), 4.40 (t, J=6.7 Hz, 2H), 3.22 (t, J=6.7 Hz, 2H), 2.95 (br s, 4H), 2.78 (s, 3H), 1.54-1.43 (m, 4H), 1.24 (s, 9H), 0.96 (s, 6H).

Alternative Procedure:

To a solution of (S)-isopropyl 2-(tert-butoxy)-2-(4′-(4,4-dimethylpiperidin-1-yl)-5-(2-fluorophenethoxy)-6′-methyl-[2,3′-bipyridin]-5′-yl)acetate (210 mg, 0.355 mmol) in methanol (15 mL) was added a solution of sodium hydroxide (142 mg, 3.55 mmol) in water (3 ml). The mixture was stirred at reflux for 6 h. Then, the pH was adjusted to 8 and purified by Pre-HPLC to give desired product (S)-2-(tert-butoxy)-2-(4′-(4,4-dimethylpiperidin-1-yl)-5-(2-fluorophenethoxy)-6′-methyl-[2,3′-bipyridin]-5′-yl)acetic acid (110 mg, 0.194 mmol, 54.7% yield). LCMS [M+H]=550.2.

### Example 304

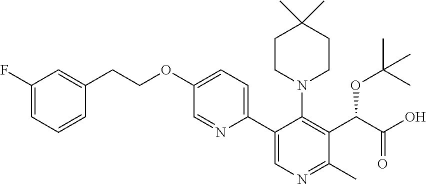 [SMILES: Cc1ncc(-c2ccc(OCCc3cccc(F)c3)cn2)c(N2CCC(C)(C)CC2)c1[C@H](OC(C)(C)C)C(=O)O]

General Procedure F was followed. The experiment afforded the desired compound, (2S)-2-(tert-butoxy)-2-[4′-(4,4-dimethylpiperidin-1-yl)-5-[2-(3-fluorophenyl)ethoxy]-6′-methyl-[2,3′-bipyridine]-5′-yl]acetic acid (19.9 mg, 68% yield, 96.4% purity). LCMS observed ion=550.3. ¹H NMR (500 MHz, METHANOL-d4) δ 8.43 (d, J=2.7 Hz, 1H), 8.35 (s, 1H), 7.60-7.57 (m, 1H), 7.56-7.53 (m, 1H), 7.37-7.32 (m, 1H), 7.17 (d, J=7.6 Hz, 1H), 7.11 (br d, J=10.1 Hz, 1H), 6.98 (t, J=8.2 Hz, 1H), 5.71 (s, 1H), 4.40 (t, J=6.4 Hz, 2H), 3.19 (t, J=6.4 Hz, 2H), 2.96 (br s, 4H), 2.78 (s, 3H), 1.54-1.42 (m, 4H), 1.24 (s, 9H), 0.96 (s, 6H).

### Example 305

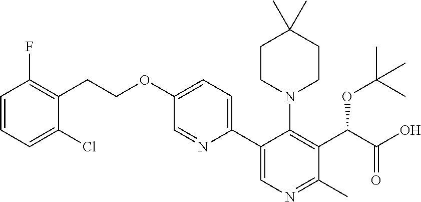 [SMILES: Cc1ncc(-c2ccc(OCCc3c(F)cccc3Cl)cn2)c(N2CCC(C)(C)CC2)c1[C@H](OC(C)(C)C)C(=O)O]

General Procedure F was followed. The experiment afforded the desired compound, (2S)-2-(tert-butoxy)-2-{5-[2-(2-chloro-6-fluorophenyl)ethoxy]-4′-(4,4-dimethylpiperidin-1-yl)-6′-methyl-[2,3′-bipyridine]-5′-yl}acetic acid (19.9 mg, 64% yield, 98.7% purity). LCMS observed ion=584.2. ¹H NMR (500 MHz, METHANOL-d4) δ 8.42 (d, J=2.4 Hz, 1H), 8.36 (s, 1H), 7.60-7.57 (m, 1H), 7.56-7.53 (m, 1H), 7.33-7.28 (m, 2H), 7.15-7.10 (m, 1H), 5.70 (s, 1H), 4.42 (t, J=6.9 Hz, 2H), 3.41-3.36 (m, 2H), 2.96 (br s, 4H), 2.79 (s, 3H), 1.54-1.44 (m, 4H), 1.24 (s, 9H), 0.97 (s, 6H).

### Example 306

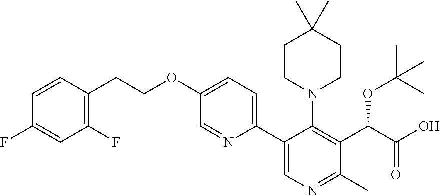 [SMILES: Cc1ncc(-c2ccc(OCCc3ccc(F)cc3F)cn2)c(N2CCC(C)(C)CC2)c1[C@H](OC(C)(C)C)C(=O)O]

General Procedure F was followed. The experiment afforded the desired compound, (2S)-2-(tert-butoxy)-2-{5-[2-(2,4-difluorophenyl)ethoxy]-4′-(4,4-dimethylpiperidin-1-yl)-6′-methyl-[2,3′-bipyridine]-5′-yl}acetic acid (21.6 mg, 71% yield, 98.6% purity). LCMS observed ion=568.3. ¹H NMR (500 MHz, METHANOL-d4) δ 8.42 (d, J=2.7 Hz, 1H), 8.35 (s, 1H), 7.59-7.56 (m, 1H), 7.56-7.52 (m, 1H), 7.47-7.41 (m, 1H), 6.98-6.93 (m, 2H), 5.71 (s, 1H), 4.38 (t, J=6.6 Hz, 2H), 3.20 (t, J=6.6 Hz, 2H), 2.95 (br s, 4H), 2.78 (s, 3H), 1.54-1.44 (m, 4H), 1.24 (s, 9H), 0.96 (s, 6H).

### Example 307

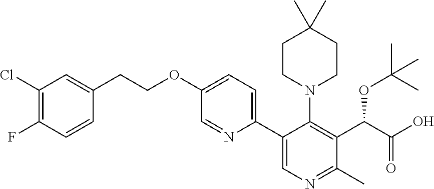 [SMILES: Cc1ncc(-c2ccc(OCCc3ccc(F)c(Cl)c3)cn2)c(N2CCC(C)(C)CC2)c1[C@H](OC(C)(C)C)C(=O)O]

General Procedure F was followed. The experiment afforded the desired compound, (2S)-2-(tert-butoxy)-2-{5-[2-(3-chloro-4-fluorophenyl)ethoxy]-4′-(4,4-dimethylpiperidin-1-yl)-6′-methyl-[2,3′-bipyridine]-5′-yl}acetic acid (18.5 mg, 59% yield, 100% purity). LCMS observed ion=584.2. ¹H NMR (500 MHz, METHANOL-d4) δ 8.34 (d, J=2.7 Hz, 1H), 8.10 (s, 1H), 7.53 (dd, J=8.5, 2.7 Hz, 1H), 7.49 (dd, J=7.0, 2.1 Hz, 1H), 7.44 (d, J=8.5 Hz, 1H), 7.32 (t, J=6.6 Hz, 1H), 7.19 (t, J=8.9 Hz, 1H), 5.77 (s, 1H), 4.36 (t, J=6.4 Hz, 2H), 3.15 (t, J=6.3 Hz, 2H), 3.23-2.85 (m, 2H), 2.77 (br s, 2H), 2.64 (s, 3H), 1.42 (br s, 4H), 1.19 (s, 9H), 0.89 (s, 6H).

### Example 308

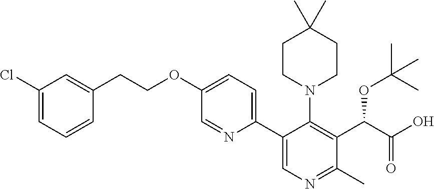 [SMILES: Cc1ncc(-c2ccc(OCCc3cccc(Cl)c3)cn2)c(N2CCC(C)(C)CC2)c1[C@H](OC(C)(C)C)C(=O)O]

General Procedure F was followed. The experiment afforded the desired compound, (2S)-2-(tert-butoxy)-2-{5-[2-(3-chlorophenyl)ethoxy]-4′-(4,4-dimethylpiperidin-1-yl)-6′-methyl-[2,3′-bipyridine]-5′-yl}acetic acid (25.7 mg, 85% yield, 98.7% purity). LCMS observed ion=566.2. ¹H NMR (500 MHz, METHANOL-d4) δ 8.43 (d, J=2.7 Hz, 1H), 8.36 (s, 1H), 7.60-7.57 (m, 1H), 7.57-7.54 (m, 1H), 7.39 (s, 1H), 7.34-7.25 (m, 3H), 5.70 (s, 1H), 4.40 (t, J=6.6 Hz, 2H), 3.17 (t, J=6.4 Hz, 2H), 2.96 (br s, 4H), 2.79 (s, 3H), 1.49 (br d, J=4.0 Hz, 4H), 1.24 (s, 9H), 0.96 (s, 6H).

Alternative Procedure:

To a solution of (S)-isopropyl 2-(tert-butoxy)-2-(5-(3-chlorophenethoxy)-4′-(4,4-dimethylpiperidin-1-yl)-6′-methyl-[2,3′-bipyridin]-5′-yl)acetate (300 mg, 0.493 mmol) in methanol (1 mL) and trifluoroethanol (1.000 mL) was added sodium hydroxide (4.93 mL, 4.93 mmol) and stirred for 20 hours at 100° C. Then, the reaction mixture was neutralized with HCl (1N, 0.5 ml) and purified by HPLC to give desired product (S)-2-(tert-butoxy)-2-(5-(3-chlorophenethoxy)-4′-(4,4-dimethylpiperidin-1-yl)-6′-methyl-[2,3′-bipyridin]-5′-yl)acetic acid (95.7 mg, 0.165 mmol, 33.4% yield) as white solid. LCMS [M+H]=566.2.

### Example 309

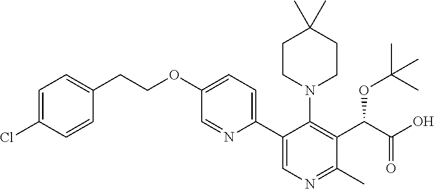 [SMILES: Cc1ncc(-c2ccc(OCCc3ccc(Cl)cc3)cn2)c(N2CCC(C)(C)CC2)c1[C@H](OC(C)(C)C)C(=O)O]

General Procedure F was followed. The experiment afforded the desired compound, (2S)-2-(tert-butoxy)-2-{5-[2-(4-chlorophenyl)ethoxy]-4′-(4,4-dimethylpiperidin-1-yl)-6′-methyl-[2,3′-bipyridine]-5′-yl}acetic acid (26.7 mg, 89% yield, 98.7% purity). LCMS observed ion=566.3. ¹H NMR (500 MHz, METHANOL-d4) δ 8.42 (d, J=2.7 Hz, 1H), 8.35 (s, 1H), 7.59-7.56 (m, 1H), 7.56-7.53 (m, 1H), 7.36-7.31 (m, 4H), 5.70 (s, 1H), 4.38 (t, J=6.6 Hz, 2H), 3.16 (t, J=6.4 Hz, 2H), 2.96 (br s, 4H), 2.79 (s, 3H), 1.49 (br d, J=4.3 Hz, 4H), 1.24 (s, 9H), 0.96 (s, 6H).

### Example 310

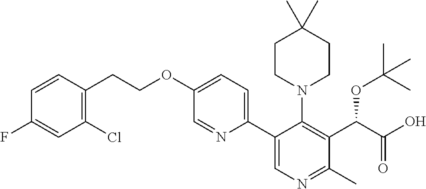 [SMILES: Cc1ncc(-c2ccc(OCCc3ccc(F)cc3Cl)cn2)c(N2CCC(C)(C)CC2)c1[C@H](OC(C)(C)C)C(=O)O]

General Procedure F was followed. The experiment afforded the desired compound, (2S)-2-(tert-butoxy)-2-{5-[2-(2-chloro-4-fluorophenyl)ethoxy]-4′-(4,4-dimethylpiperidin-1-yl)-6′-methyl-[2,3′-bipyridine]-5′-yl}acetic acid (23.7 mg, 76% yield, 98.5% purity). LCMS observed ion=584.1. ¹H NMR (500 MHz, METHANOL-d4) δ 8.43 (d, J=2.7 Hz, 1H), 8.36 (s, 1H), 7.60-7.57 (m, 1H), 7.55 (d, J=8.5 Hz, 1H), 7.49 (dd, J=8.5, 6.1 Hz, 1H), 7.26 (dd, J=8.7, 2.6 Hz, 1H), 7.08 (td, J=8.4, 2.7 Hz, 1H), 5.70 (s, 1H), 4.41 (t, J=6.6 Hz, 2H), 3.31-3.29 (m, 2H), 2.96 (br s, 4H), 2.79 (s, 3H), 1.55-1.43 (m, 4H), 1.24 (s, 9H), 0.96 (s, 6H).

### Example 311

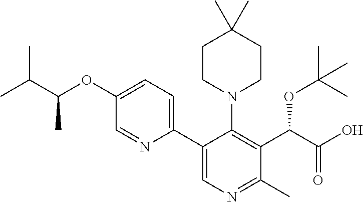 [SMILES: Cc1ncc(-c2ccc(O[C@@H](C)C(C)C)cn2)c(N2CCC(C)(C)CC2)c1[C@H](OC(C)(C)C)C(=O)O]

General Procedure F was followed. The experiment afforded the desired compound, (2S)-2-(tert-butoxy)-2-[4′-(4,4-dimethylpiperidin-1-yl)-6′-methyl-5-{[(2S)-3-methylbutan-2-yl]oxy}-[2,3′-bipyridine]-5′-yl]acetic acid (13.1 mg, 49% yield, 96.4% purity). LCMS observed ion=498.3. ¹H NMR (500 MHz, METHANOL-d4) δ 8.41 (d, J=2.7 Hz, 1H), 8.39-8.35 (m, 1H), 7.59 (dd, J=8.5, 3.1 Hz, 1H), 7.55 (d, J=8.9 Hz, 1H), 5.73 (s, 1H), 4.44 (quin, J=6.1 Hz, 1H), 2.97 (br s, 4H), 2.79 (s, 3H), 2.04-1.95 (m, 1H), 1.53-1.44 (m, 4H), 1.32 (d, J=6.4 Hz, 3H), 1.27-1.23 (m, 9H), 1.07 (d, J=6.7 Hz, 3H), 1.04 (d, J=6.7 Hz, 3H), 0.96 (s, 6H).

### Example 312

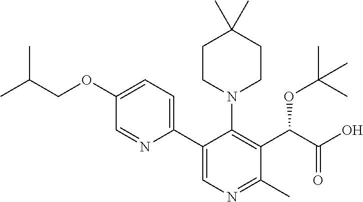 [SMILES: Cc1ncc(-c2ccc(OCC(C)C)cn2)c(N2CCC(C)(C)CC2)c1[C@H](OC(C)(C)C)C(=O)O]

General Procedure F was followed. The experiment afforded the desired compound, (2S)-2-(tert-butoxy)-2-[4′-(4,4-dimethylpiperidin-1-yl)-6′-methyl-5-(2-methylpropoxy)-[2,3′-bipyridine]-5′-yl]acetic acid (17.6 mg, 68% yield, 100% purity). LCMS observed ion=484.3. ¹H NMR (500 MHz, METHANOL-d4) δ 8.37 (d, J=2.7 Hz, 1H), 8.17 (s, 1H), 7.55 (dd, J=8.7, 2.9 Hz, 1H), 7.47 (d, J=8.5 Hz, 1H), 5.84 (s, 1H), 3.92 (d, J=6.4 Hz, 2H), 2.83 (br s, 4H), 2.66 (s, 3H), 2.15 (dt, J=13.3, 6.8 Hz, 1H), 1.44 (br s, 4H), 1.21 (s, 9H), 1.10 (d, J=6.7 Hz, 6H), 0.91 (s, 6H).

Alternative Procedure:

To a stirred solution of (S)-isopropyl 2-(tert-butoxy)-2-(4′-(4,4-dimethylpiperidin-1-yl)-5-isobutoxy-6′-methyl-[2,3′-bipyridin]-5′-yl)acetate (100 mg, 0.190 mmol) in methanol (5 mL) was added NaOH (152 mg, 3.80 mmol) in water (1.00 mL) and stirred at 90° C. for 18 hours. The mixture was concentrated and residue was diluted with EtOAc (50 ml), water (20 ml) and adjusted the pH to 7 with acetic acid. Then, the organic layer was separated and washed with brine (20 ml×3), dried over MgSO₄ and concentrated to afford (S)-2-(tert-butoxy)-2-(4′-(4,4-dimethylpiperidin-1-yl)-5-isobutoxy-6′-methyl-[2,3′-bipyridin]-5′-yl)acetic acid (80 mg, 0.163 mmol, 86% yield) as a white foam. LCMS (M+H)=484.

### Example 313

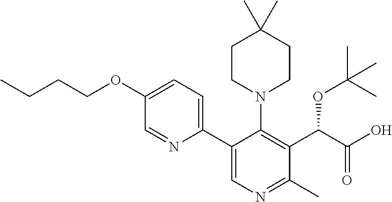 [SMILES: CCCCOc1ccc(-c2cnc(C)c([C@H](OC(C)(C)C)C(=O)O)c2N2CCC(C)(C)CC2)nc1]

General Procedure F was followed. The experiment afforded the desired compound, (2S)-2-(tert-butoxy)-2-[5-butoxy-4′-(4,4-dimethylpiperidin-1-yl)-6′-methyl-[2,3′-bipyridine]-5′-yl]acetic acid (20.1 mg, 78% yield, 100% purity). LCMS observed ion=484.3. ¹H NMR (500 MHz, METHANOL-d4) δ 8.37 (d, J=2.7 Hz, 1H), 8.18 (s, 1H), 7.55 (dd, J=8.5, 3.1 Hz, 1H), 7.47 (d, J=8.9 Hz, 1H), 5.84 (s, 1H), 4.16 (t, J=6.4 Hz, 2H), 2.84 (br s, 4H), 2.66 (s, 3H), 1.88-1.82 (m, 2H), 1.57 (sxt, J=7.4 Hz, 2H), 1.44 (br s, 4H), 1.21 (s, 9H), 1.03 (t, J=7.5 Hz, 3H), 0.91 (s, 6H).

Alternative Procedure:

To a solution of (S)-isopropyl 2-(tert-butoxy)-2-(5-butoxy-4′-(4,4-dimethylpiperidin-1-yl)-6′-methyl-[2,3′-bipyridin]-5′-yl)acetate (250 mg, 0.476 mmol) in methanol (5 mL) was added a solution of NaOH (285 mg, 7.13 mmol) in water (1 mL). The resulting mixture was stirred at 70° C. for 16 h. Then, the reaction solution was purified by Pre-HPLC to afford the desired product (S)-2-(tert-butoxy)-2-(5-butoxy-4′-(4,4-dimethylpiperidin-1-yl)-6′-methyl-[2,3′-bipyridin]-5′-yl)acetic acid (180 mg, 0.361 mmol, 76% yield) as white solid. LCMS (M+H)=484.3.

### Example 314

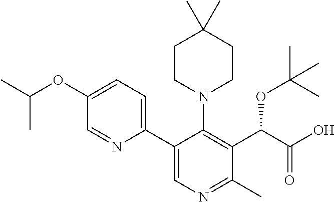 [SMILES: Cc1ncc(-c2ccc(OC(C)C)cn2)c(N2CCC(C)(C)CC2)c1[C@H](OC(C)(C)C)C(=O)O]

General Procedure F was followed. The experiment afforded the desired compound, (2S)-2-(tert-butoxy)-2-[4′-(4,4-dimethylpiperidin-1-yl)-6′-methyl-5-(propan-2-yloxy)-[2,3′-bipyridine]-5′-yl]acetic acid (17.3 mg, 69% yield, 100% purity). LCMS observed ion=470.1. ¹H NMR (500 MHz, METHANOL-d4) δ 8.40 (d, J=2.7 Hz, 1H), 8.38 (s, 1H), 7.58 (dd, J=8.9, 2.7 Hz, 1H), 7.55 (d, J=8.5 Hz, 1H), 5.72 (s, 1H), 4.81-4.78 (m, 1H), 2.98 (br s, 4H), 2.79 (s, 3H), 1.53-1.45 (m, 4H), 1.41 (dd, J=6.0, 3.5 Hz, 6H), 1.25 (s, 9H), 0.97 (s, 5H), 0.96-0.95 (m, 1H).

### Example 315

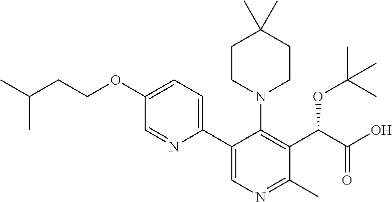 [SMILES: Cc1ncc(-c2ccc(OCCC(C)C)cn2)c(N2CCC(C)(C)CC2)c1[C@H](OC(C)(C)C)C(=O)O]

General Procedure F was followed. The experiment afforded the desired compound, (2S)-2-(tert-butoxy)-2-[4′-(4,4-dimethylpiperidin-1-yl)-6′-methyl-5-(3-methylbutoxy)-[2,3′-bipyridine]-5′-yl]acetic acid (14.4 mg, 54% yield, 98.8% purity). LCMS Method 2: retention time=2.06 min.; observed ion=498.2. ¹H NMR (500 MHz, METHANOL-d4) δ 8.37 (d, J=2.7 Hz, 1H), 8.17 (s, 1H), 7.56 (dd, J=8.5, 2.7 Hz, 1H), 7.47 (d, J=8.5 Hz, 1H), 5.84 (s, 1H), 4.19 (t, J=6.6 Hz, 2H), 2.83 (br s, 1H), 3.15-2.75 (m, 4H), 2.66 (s, 3H), 1.90 (spt, J=6.6 Hz, 1H), 1.76 (q, J=6.7 Hz, 2H), 1.44 (br s, 4H), 1.21 (s, 9H), 1.02 (d, J=6.7 Hz, 6H), 0.91 (s, 6H).

Alternative Procedure:

To a stirred solution of (S)-isopropyl 2-(tert-butoxy)-2-(4′-(4,4-dimethylpiperidin-1-yl)-5-(isopentyloxy)-6′-methyl-[2,3′-bipyridin]-5′-yl)acetate (240 mg, 0.445 mmol) in methanol (10 mL) was added a solution of sodium hydroxide (534 mg, 13.34 mmol) in water (3.00 mL). The mixture was stirred overnight at 75° C., cooled, pH was adjusted to 8 and concentrated. The residue was purified by Pre-HPLC to afford the desired product (S)-2-(tert-butoxy)-2-(4′-(4,4-dimethylpiperidin-1-yl)-5-(isopentyloxy)-6′-methyl-[2,3′-bipyridin]-5′-yl)acetic acid (153 mg, 0.307 mmol, 69.1% yield) as a white solid. LCMS [M+H]=498.4.

### Example 316

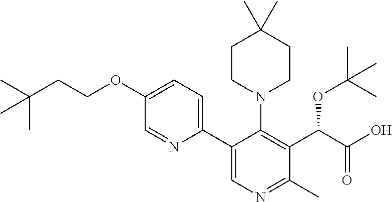 [SMILES: Cc1ncc(-c2ccc(OCCC(C)(C)C)cn2)c(N2CCC(C)(C)CC2)c1[C@H](OC(C)(C)C)C(=O)O]

General Procedure F was followed. The experiment afforded the desired compound, (2S)-2-(tert-butoxy)-2-[5-(3,3-dimethylbutoxy)-4′-(4,4-dimethylpiperidin-1-yl)-6′-methyl-[2,3′-bipyridine]-5′-yl]acetic acid (13.1 mg, 48% yield, 98.8% purity). LCMS observed ion=512.2. ¹H NMR (500 MHz, METHANOL-d4) δ 8.36 (d, J=2.7 Hz, 1H), 8.17 (s, 1H), 7.56 (dd, J=8.5, 2.7 Hz, 1H), 7.48 (d, J=8.9 Hz, 1H), 5.85 (s, 1H), 4.23 (t, J=7.0 Hz, 2H), 3.14-2.72 (m, 4H), 2.66 (s, 3H), 1.81 (t, J=7.0 Hz, 2H), 1.44 (br s, 4H), 1.21 (s, 9H), 1.05 (s, 9H), 0.91 (s, 6H).

### Example 317

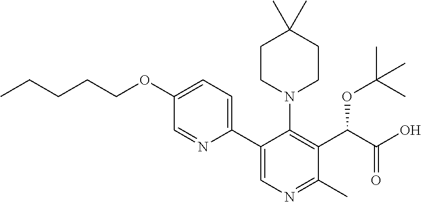 [SMILES: CCCCCOc1ccc(-c2cnc(C)c([C@H](OC(C)(C)C)C(=O)O)c2N2CCC(C)(C)CC2)nc1]

General Procedure F was followed. The experiment afforded the desired compound, (2S)-2-(tert-butoxy)-2-[4′-(4,4-dimethylpiperidin-1-yl)-6′-methyl-5-(pentyloxy)-[2,3′-bipyridine]-5′-yl]acetic acid (11.6 mg, 44% yield, 100% purity). LCMS observed ion=498.3. ¹H NMR (500 MHz, METHANOL-d4) δ 8.37 (d, J=2.7 Hz, 1H), 8.17 (s, 1H), 7.55 (dd, J=8.5, 2.7 Hz, 1H), 7.47 (d, J=8.5 Hz, 1H), 5.84 (s, 1H), 4.15 (t, J=6.4 Hz, 2H), 3.15-2.72 (m, 4H), 2.66 (s, 3H), 1.90-1.84 (m, 2H), 1.56-1.41 (m, 8H), 1.21 (s, 9H), 0.99 (t, J=7.2 Hz, 3H), 0.91 (s, 6H).

Alternative Procedure:

To a stirred solution of (S)-isopropyl 2-(tert-butoxy)-2-(4′-(4,4-dimethylpiperidin-1-yl)-6′-methyl-5-(pentyloxy)-[2,3′-bipyridin]-5′-yl)acetate (220 mg, 0.408 mmol) in methanol (10 mL) was added a solution of sodium hydroxide (489 mg, 12.23 mmol) in water (3.00 mL). The mixture was stirred overnight at 75° C. and cooled, pH was adjusted to 8 and concentrated. The residue was purified by Pre-HPLC to afford the desired product (S)-2-(tert-butoxy)-2-(4′-(4,4-dimethylpiperidin-1-yl)-6′-methyl-5-(pentyloxy)-[2,3′-bipyridin]-5′-yl)acetic acid (121 mg, 0.238 mmol, 58.5% yield) as a white solid. LCMS [M+H]=498.4.

### Example 318

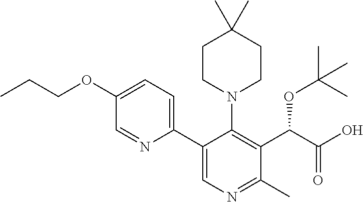 [SMILES: CCCOc1ccc(-c2cnc(C)c([C@H](OC(C)(C)C)C(=O)O)c2N2CCC(C)(C)CC2)nc1]

General Procedure F was followed. The experiment afforded the desired compound, (2S)-2-(tert-butoxy)-2-[4′-(4,4-dimethylpiperidin-1-yl)-6′-methyl-5-propoxy-[2,3′-bipyridine]-5′-yl]acetic acid (5.2 mg, 20% yield, 97.5% purity). LCMS observed ion=470.1. ¹H NMR (500 MHz, METHANOL-d4) δ 8.37 (d, J=2.7 Hz, 1H), 8.18 (s, 1H), 7.56 (dd, J=8.5, 2.7 Hz, 1H), 7.48 (d, J=8.5 Hz, 1H), 5.84 (s, 1H), 4.12 (t, J=6.4 Hz, 2H), 3.13-2.72 (m, 4H), 2.67 (s, 3H), 1.92-1.85 (m, 2H), 1.45 (br s, 4H), 1.22 (s, 9H), 1.11 (t, J=7.5 Hz, 3H), 0.92 (s, 6H).

### Example 319

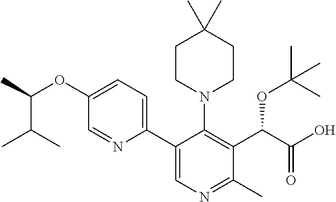 [SMILES: Cc1ncc(-c2ccc(O[C@H](C)C(C)C)cn2)c(N2CCC(C)(C)CC2)c1[C@H](OC(C)(C)C)C(=O)O]

General Procedure F was followed. The experiment afforded the desired compound, (2S)-2-(tert-butoxy)-2-[4′-(4,4-dimethylpiperidin-1-yl)-6′-methyl-5-{[(2R)-3-methylbutan-2-yl]oxy}-[2,3′-bipyridine]-5′-yl]acetic acid (9.3 mg, 35% yield, 98.6% purity). LCMS observed ion=498.2. ¹H NMR (500 MHz, METHANOL-d4) δ 8.35 (d, J=2.4 Hz, 1H), 8.19 (s, 1H), 7.57 (dd, J=8.7, 2.9 Hz, 1H), 7.47 (d, J=8.5 Hz, 1H), 5.82 (s, 1H), 4.43 (quin, J=6.1 Hz, 1H), 2.83 (br s, 4H), 2.67 (s, 3H), 2.01 (sxt, J=7.0 Hz, 1H), 1.44 (br s, 4H), 1.32 (d, J=6.4 Hz, 3H), 1.21 (s, 9H), 1.06 (d, J=7.0 Hz, 3H), 1.03 (d, J=7.0 Hz, 3H), 0.91 (s, 6H).

### Example 320

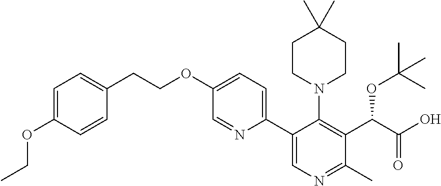 [SMILES: CCOc1ccc(CCOc2ccc(-c3cnc(C)c([C@H](OC(C)(C)C)C(=O)O)c3N3CCC(C)(C)CC3)nc2)cc1]

General Procedure F was followed. The experiment afforded the desired compound, (2S)-2-(tert-butoxy)-2-[4′-(4,4-dimethylpiperidin-1-yl)-5-[2-(4-ethoxyphenyl)ethoxy]-6′-methyl-[2,3′-bipyridine]-5′-yl]acetic acid (18.3 mg, 60% yield, 100% purity). LCMS observed ion=576.3. ¹H NMR (500 MHz, METHANOL-d4) δ 8.32 (d, J=2.7 Hz, 1H), 8.09 (s, 1H), 7.52 (dd, J=8.7, 2.9 Hz, 1H), 7.43 (d, J=8.5 Hz, 1H), 7.26-7.23 (m, J=8.5 Hz, 2H), 6.89-6.86 (m, J=8.5 Hz, 2H), 5.74 (s, 1H), 4.32 (t, J=6.6 Hz, 2H), 4.03 (q, J=6.8 Hz, 2H), 3.08 (t, J=6.9 Hz, 2H), 2.84-2.67 (m, 2H), 2.64 (s, 3H), 1.49-1.32 (m, 7H), 1.18 (s, 9H), 0.89 (s, 6H).

### Example 321

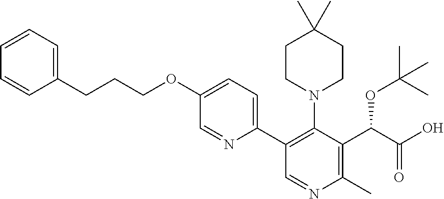 [SMILES: Cc1ncc(-c2ccc(OCCCc3ccccc3)cn2)c(N2CCC(C)(C)CC2)c1[C@H](OC(C)(C)C)C(=O)O]

General Procedure F was followed. The experiment afforded the desired compound, (2S)-2-(tert-butoxy)-2-[4′-(4,4-dimethylpiperidin-1-yl)-6′-methyl-5-(3-phenylpropoxy)-[2,3′-bipyridine]-5′-yl]acetic acid (10.1 mg, 34% yield, 100% purity). LCMS observed ion=546.3. ¹H NMR (500 MHz, METHANOL-d4) δ 8.38 (d, J=2.7 Hz, 1H), 8.22 (s, 1H), 7.55 (dd, J=8.5, 2.7 Hz, 1H), 7.49 (d, J=8.9 Hz, 1H), 7.31-7.27 (m, 2H), 7.25 (d, J=6.7 Hz, 2H), 7.21-7.18 (m, 1H), 5.80 (s, 1H), 4.14 (t, J=6.3 Hz, 2H), 2.86 (br t, J=7.6 Hz, 2H), 3.16-2.77 (m, 4H), 2.68 (s, 3H), 2.20-2.14 (m, 2H), 1.45 (br s, 4H), 1.22 (s, 9H), 0.92 (s, 6H).

### Example 322

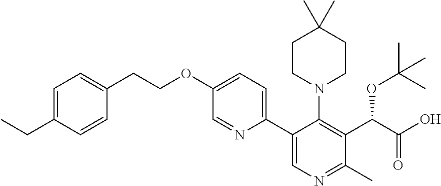 [SMILES: CCc1ccc(CCOc2ccc(-c3cnc(C)c([C@H](OC(C)(C)C)C(=O)O)c3N3CCC(C)(C)CC3)nc2)cc1]

General Procedure F was followed. The experiment afforded the desired compound, (2S)-2-(tert-butoxy)-2-[4′-(4,4-dimethylpiperidin-1-yl)-5-[2-(4-ethylphenyl)ethoxy]-6′-methyl-[2,3′-bipyridine]-5′-yl]acetic acid (14.1 mg, 48% yield, 98.1% purity). LCMS observed ion=560.3. ¹H NMR (500 MHz, DMSO-d6) δ 8.38 (d, J=2.4 Hz, 1H), 8.13 (s, 1H), 7.52 (dd, J=8.7, 2.6 Hz, 1H), 7.44 (d, J=8.9 Hz, 1H), 7.24 (d, J=7.6 Hz, 2H), 7.16 (d, J=7.9 Hz, 2H), 5.88 (s, 1H), 4.36-4.25 (m, 2H), 3.30 (br s, 4H), 3.04 (t, J=6.9 Hz, 2H), 2.57 (q, J=7.9 Hz, 2H), 2.49-2.47 (m, 3H), 2.07 (br s, 1H), 1.62-1.45 (m, 1H), 1.30 (br s, 2H), 1.16 (t, J=7.6 Hz, 3H), 1.12 (s, 9H), 0.88 (br s, 3H), 0.77 (br s, 3H).

### Example 323

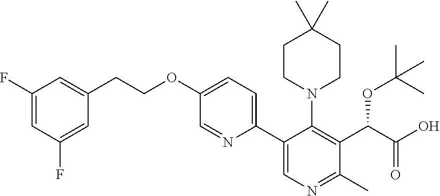 [SMILES: Cc1ncc(-c2ccc(OCCc3cc(F)cc(F)c3)cn2)c(N2CCC(C)(C)CC2)c1[C@H](OC(C)(C)C)C(=O)O]

General Procedure F was followed. The experiment afforded the desired compound, (2S)-2-(tert-butoxy)-2-{5-[2-(3,5-difluorophenyl)ethoxy]-4′-(4,4-dimethylpiperidin-1-yl)-6′-methyl-[2,3′-bipyridine]-5′-yl}acetic acid (5.2 mg, 17% yield, 99.3% purity). LCMS observed ion=568.2. ¹H NMR (500 MHz, METHANOL-d4) δ 8.34 (d, J=2.4 Hz, 1H), 8.09 (s, 1H), 7.55 (dd, J=8.9, 2.7 Hz, 1H), 7.44 (d, J=8.5 Hz, 1H), 7.00 (br d, J=7.0 Hz, 2H), 6.83 (br t, J=8.5 Hz, 1H), 5.75 (s, 1H), 4.39 (t, J=6.4 Hz, 2H), 3.19 (t, J=6.3 Hz, 2H), 2.71 (br s, 2H), 2.64 (s, 3H), 1.42 (br s, 4H), 1.18 (s, 9H), 0.89 (s, 6H).

Alternative Procedure:

To a solution of (S)-isopropyl 2-(tert-butoxy)-2-(5-(3,5-difluorophenethoxy)-4′-(4,4-dimethylpiperidin-1-yl)-6′-methyl-[2,3′-bipyridin]-5′-yl)acetate (200 mg, 0.328 mmol) in methanol (10 mL) was added a solution of sodium hydroxide (394 mg, 9.84 mmol) in water (3.00 mL). The mixture was stirred overnight at 75° C. and pH was adjusted to 8. The mixture was purified by Pre-HPLC to afford the desired product (S)-2-(tert-butoxy)-2-(5-(3,5-difluorophenethoxy)-4′-(4,4-dimethylpiperidin-1-yl)-6′-methyl-[2,3′-bipyridin]-5′-yl)acetic acid (60 mg, 0.106 mmol, 32.2% yield) as a white solid. LCMS [M+H]=568.2.

### Example 324

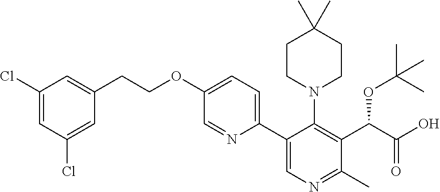 [SMILES: Cc1ncc(-c2ccc(OCCc3cc(Cl)cc(Cl)c3)cn2)c(N2CCC(C)(C)CC2)c1[C@H](OC(C)(C)C)C(=O)O]

General Procedure F was followed. The experiment afforded the desired compound, (2S)-2-(tert-butoxy)-2-{5-[2-(3,5-dichlorophenyl)ethoxy]-4′-(4,4-dimethylpiperidin-1-yl)-6′-methyl-[2,3′-bipyridine]-5′-yl}acetic acid (1.3 mg, 4% yield, 98.5% purity). LCMS observed ion=600.2. ¹H NMR (500 MHz, METHANOL-d4) δ 8.37 (d, J=2.4 Hz, 1H), 8.17 (s, 1H), 7.56 (dd, J=8.7, 2.9 Hz, 1H), 7.47 (d, J=8.5 Hz, 1H), 7.37-7.33 (m, 3H), 5.81 (s, 1H), 4.39 (t, J=6.3 Hz, 2H), 3.17 (t, J=6.3 Hz, 2H), 2.80 (br s, 4H), 2.66 (s, 3H), 1.44 (br s, 4H), 1.21 (s, 9H), 0.90 (s, 6H).

### Example 325

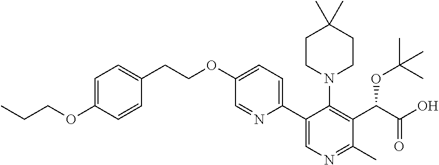 [SMILES: CCCOc1ccc(CCOc2ccc(-c3cnc(C)c([C@H](OC(C)(C)C)C(=O)O)c3N3CCC(C)(C)CC3)nc2)cc1]

General Procedure F was followed. The experiment afforded the desired compound, (2S)-2-(tert-butoxy)-2-[4′-(4,4-dimethylpiperidin-1-yl)-6′-methyl-5-[2-(4-propoxyphenyl)ethoxy]-[2,3′-bipyridine]-5′-yl]acetic acid (16.5 mg, 53% yield, 100% purity). LCMS observed ion=590.4. ¹H NMR (500 MHz, METHANOL-d4) δ 8.35 (d, J=2.7 Hz, 1H), 8.17 (s, 1H), 7.54 (dd, J=8.5, 2.7 Hz, 1H), 7.46 (d, J=8.5 Hz, 1H), 7.26-7.23 (m, J=8.5 Hz, 2H), 6.89-6.86 (m, J=8.5 Hz, 2H), 5.81 (s, 1H), 4.32 (t, J=6.6 Hz, 2H), 3.93 (t, J=6.6 Hz, 2H), 3.09 (t, J=6.7 Hz, 2H), 2.81 (br s, 2H), 2.66 (s, 3H), 1.83-1.76 (m, 2H), 1.44 (br s, 4H), 1.21 (s, 9H), 1.05 (t, J=7.5 Hz, 3H), 0.91 (s, 6H).

### Example 326

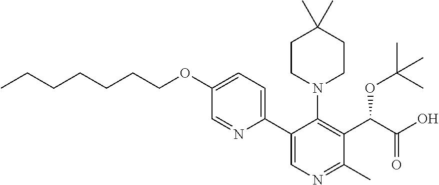 [SMILES: CCCCCCCOc1ccc(-c2cnc(C)c([C@H](OC(C)(C)C)C(=O)O)c2N2CCC(C)(C)CC2)nc1]

General procedure B was followed. The experiment afforded the desired compound, (2S)-2-(tert-butoxy)-2-[4′-(4,4-dimethylpiperidin-1-yl)-5-(heptyloxy)-6′-methyl-[2,3′-bipyridine]-5′-yl]acetic acid (11.1 mg, 39% yield, 100% purity). LCMS observed ion=526.3. ¹H NMR (500 MHz, METHANOL-d4) δ 8.37 (d, J=2.4 Hz, 1H), 8.20 (s, 1H), 7.56 (dd, J=8.7, 2.6 Hz, 1H), 7.48 (d, J=8.5 Hz, 1H), 5.82 (s, 1H), 4.15 (t, J=6.3 Hz, 2H), 2.84 (br s, 4H), 2.67 (s, 3H), 1.89-1.83 (m, 2H), 1.54 (quin, J=7.6 Hz, 2H), 1.49-1.40 (m, 6H), 1.37 (br s, 4H), 1.21 (s, 9H), 0.96-0.91 (m, 9H).

### Example 327

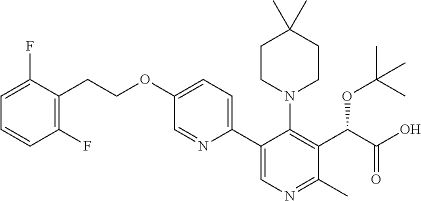 [SMILES: Cc1ncc(-c2ccc(OCCc3c(F)cccc3F)cn2)c(N2CCC(C)(C)CC2)c1[C@H](OC(C)(C)C)C(=O)O]

General Procedure F was followed. The experiment afforded the desired compound, (2S)-2-(tert-butoxy)-2-{5-[2-(2,6-difluorophenyl)ethoxy]-4′-(4,4-dimethylpiperidin-1-yl)-6′-methyl-[2,3′-bipyridine]-5′-yl}acetic acid (16.7 mg, 55% yield, 98.6% purity). LCMS observed ion=568.2. ¹H NMR (500 MHz, METHANOL-d4) δ 8.30 (d, J=2.1 Hz, 1H), 8.08 (s, 1H), 7.53 (dd, J=8.5, 2.7 Hz, 1H), 7.43 (d, J=8.5 Hz, 1H), 7.35-7.29 (m, 1H), 7.00 (t, J=7.8 Hz, 2H), 5.74 (s, 1H), 4.37 (t, J=6.7 Hz, 2H), 3.25 (br t, J=6.6 Hz, 2H), 2.75 (br s, 2H), 2.64 (s, 3H), 1.42 (br s, 4H), 1.18 (s, 9H), 0.89 (s, 6H).

### Example 328

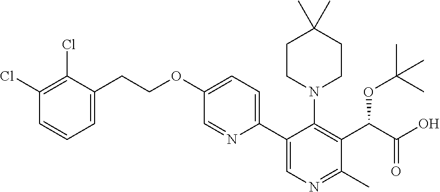 [SMILES: Cc1ncc(-c2ccc(OCCc3cccc(Cl)c3Cl)cn2)c(N2CCC(C)(C)CC2)c1[C@H](OC(C)(C)C)C(=O)O]

General Procedure F was followed. The experiment afforded the desired compound, (2S)-2-(tert-butoxy)-2-{5-[2-(2,3-dichlorophenyl)ethoxy]-4′-(4,4-dimethylpiperidin-1-yl)-6′-methyl-[2,3′-bipyridine]-5′-yl}acetic acid (6.7 mg, 21% yield, 99.3% purity). LCMS observed ion=600.2. ¹H NMR (500 MHz, METHANOL-d4) δ 8.33 (s, 1H), 8.08 (s, 1H), 7.55 (br d, J=8.2 Hz, 1H), 7.48-7.41 (m, 3H), 7.29 (t, J=7.8 Hz, 1H), 5.74 (s, 1H), 4.42 (brt, J=6.4 Hz, 2H), 3.39-3.35 (m, 2H), 2.73 (br s, 2H), 2.64 (s, 3H), 1.42 (br s, 4H), 1.18 (s, 9H), 0.89 (s, 6H).

### Example 329

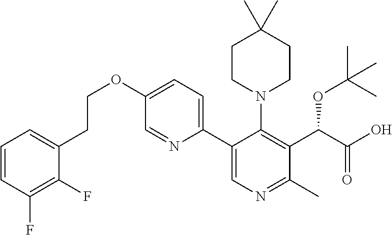 [SMILES: Cc1ncc(-c2ccc(OCCc3cccc(F)c3F)cn2)c(N2CCC(C)(C)CC2)c1[C@H](OC(C)(C)C)C(=O)O]

General Procedure F was followed. The experiment afforded the desired compound, (2S)-2-(tert-butoxy)-2-{5-[2-(2,3-difluorophenyl)ethoxy]-4′-(4,4-dimethylpiperidin-1-yl)-6′-methyl-[2,3′-bipyridine]-5′-yl}acetic acid (11.9 mg, 39% yield, 99.4% purity). LCMS observed ion=568.3. ¹H NMR (500 MHz, METHANOL-d4) δ 8.31 (d, J=2.4 Hz, 1H), 8.08 (s, 1H), 7.52 (d, J=7.7 Hz, 1H), 7.43 (d, J=8.3 Hz, 1H), 7.22-7.10 (m, 3H), 5.74 (s, 1H), 4.39 (br t, J=6.7 Hz, 2H), 3.23 (br t, J=6.4 Hz, 2H), 2.75 (br s, 2H), 2.62 (s, 3H), 1.40 (br s, 4H), 1.17 (s, 9H), 0.87 (s, 6H).

### Example 330

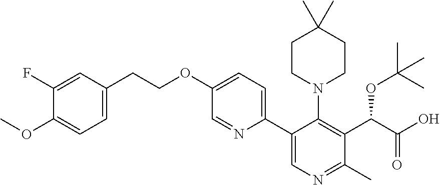 [SMILES: COc1ccc(CCOc2ccc(-c3cnc(C)c([C@H](OC(C)(C)C)C(=O)O)c3N3CCC(C)(C)CC3)nc2)cc1F]

General Procedure F was followed. The experiment afforded the desired compound, (2S)-2-(tert-butoxy)-2-[4′-(4,4-dimethylpiperidin-1-yl)-5-[2-(3-fluoro-4-methoxyphenyl)ethoxy]-6′-methyl-[2,3′-bipyridine]-5′-yl]acetic acid (21.1 mg, 68% yield, 98.6% purity). LCMS observed ion=580.3. ¹H NMR (500 MHz, METHANOL-d4) δ 8.32 (s, 1H), 8.08 (s, 1H), 7.53 (d, J=8.1 Hz, 1H), 7.43 (br d, J=8.5 Hz, 1H), 7.13-7.03 (m, 3H), 5.74 (s, 1H), 4.33 (br t, J=6.4 Hz, 2H), 3.87 (s, 3H), 3.09 (br t, J=6.6 Hz, 2H), 2.74 (br s, 2H), 2.64 (s, 3H), 1.41 (br s, 4H), 1.18 (s, 9H), 0.88 (s, 6H).

### Example 331

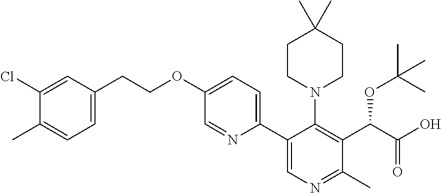 [SMILES: Cc1ccc(CCOc2ccc(-c3cnc(C)c([C@H](OC(C)(C)C)C(=O)O)c3N3CCC(C)(C)CC3)nc2)cc1Cl]

General Procedure F was followed. The experiment afforded the desired compound, (2S)-2-(tert-butoxy)-2-{5-[2-(3-chloro-4-methylphenyl)ethoxy]-4′-(4,4-dimethylpiperidin-1-yl)-6′-methyl-[2,3′-bipyridine]-5′-yl}acetic acid (14.8 mg, 48% yield, 99.4% purity). LCMS observed ion=580.3. ¹H NMR (500 MHz, METHANOL-d4) δ 8.33 (s, 1H), 8.11 (br s, 1H), 7.54 (dd, J=8.7, 2.6 Hz, 1H), 7.44 (d, J=8.5 Hz, 1H), 7.36 (s, 1H), 7.25 (d, J=7.9 Hz, 1H), 7.19 (br d, J=7.6 Hz, 1H), 5.76 (br s, 1H), 4.35 (t, J=6.4 Hz, 2H), 3.12 (br t, J=6.4 Hz, 2H), 2.76 (br s, 2H), 2.64 (s, 3H), 2.35 (s, 3H), 1.42 (br s, 4H), 1.19 (s, 9H), 0.89 (s, 6H).

### Example 332

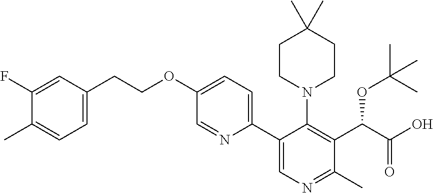 [SMILES: Cc1ccc(CCOc2ccc(-c3cnc(C)c([C@H](OC(C)(C)C)C(=O)O)c3N3CCC(C)(C)CC3)nc2)cc1F]

General Procedure F was followed. The experiment afforded the desired compound, (2S)-2-(tert-butoxy)-2-[4′-(4,4-dimethylpiperidin-1-yl)-5-[2-(3-fluoro-4-methylphenyl)ethoxy]-6′-methyl-[2,3′-bipyridine]-5′-yl]acetic acid (10 mg, 33% yield, 100% purity). LCMS observed ion=564.3. ¹H NMR (500 MHz, METHANOL-d4) δ 8.36 (d, J=2.1 Hz, 1H), 8.17 (s, 1H), 7.55 (dd, J=8.4, 2.6 Hz, 1H), 7.47 (d, J=8.5 Hz, 1H), 7.18 (t, J=7.7 Hz, 1H), 7.07-7.02 (m, 2H), 5.81 (br s, 1H), 4.36 (t, J=6.6 Hz, 2H), 3.13 (br t, J=6.4 Hz, 2H), 2.82 (br s, 2H), 2.66 (s, 3H), 2.25 (s, 3H), 1.44 (br s, 4H), 1.21 (s, 9H), 0.91 (s, 6H).

### Example 333

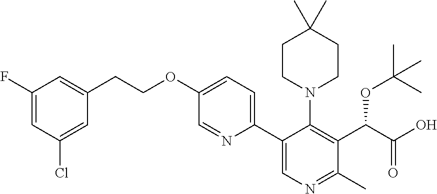 [SMILES: Cc1ncc(-c2ccc(OCCc3cc(F)cc(Cl)c3)cn2)c(N2CCC(C)(C)CC2)c1[C@H](OC(C)(C)C)C(=O)O]

General Procedure F was followed. The experiment afforded the desired compound, (2S)-2-(tert-butoxy)-2-{5-[2-(3-chloro-5-fluorophenyl)ethoxy]-4′-(4,4-dimethylpiperidin-1-yl)-6′-methyl-[2,3′-bipyridine]-5′-yl}acetic acid (2.5 mg, 8% yield, 95.9% purity). LCMS observed ion=584.2. ¹H NMR (500 MHz, METHANOL-d4) δ 8.43 (s, 1H), 8.36 (s, 1H), 7.61-7.54 (m, 2H), 7.25 (s, 1H), 7.10 (br t, J=8.2 Hz, 2H), 5.70 (s, 1H), 4.41 (t, J=6.3 Hz, 2H), 3.19 (br t, J=6.1 Hz, 2H), 2.96 (br s, 4H), 2.78 (s, 3H), 1.48 (br s, 4H), 1.24 (s, 9H), 0.96 (s, 6H).

### Example 334

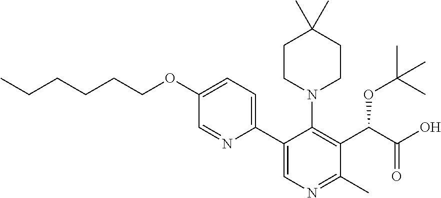 [SMILES: CCCCCCOc1ccc(-c2cnc(C)c([C@H](OC(C)(C)C)C(=O)O)c2N2CCC(C)(C)CC2)nc1]

General Procedure F was followed. The experiment afforded the desired compound, (2S)-2-(tert-butoxy)-2-[4′-(4,4-dimethylpiperidin-1-yl)-5-(hexyloxy)-6′-methyl-[2,3′-bipyridine]-5′-yl]acetic acid (10.9 mg, 40% yield, 97.3% purity). LCMS observed ion=512.3. ¹H NMR (500 MHz, METHANOL-d4) δ 8.37 (d, J=2.1 Hz, 1H), 8.19 (s, 1H), 7.56 (dd, J=8.4, 2.3 Hz, 1H), 7.48 (d, J=8.2 Hz, 1H), 5.82 (s, 1H), 4.15 (t, J=6.4 Hz, 2H), 2.84 (br s, 2H), 3.15-2.76 (m, 2H), 2.67 (s, 3H), 1.90-1.82 (m, 2H), 1.54 (br s, 2H), 1.49-1.38 (m, 8H), 1.21 (s, 9H), 0.98-0.94 (m, 3H), 0.92 (s, 6H).

### Example 335

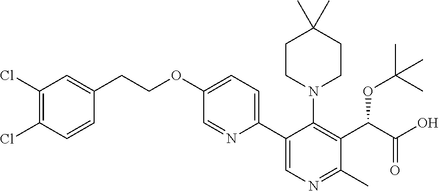 [SMILES: Cc1ncc(-c2ccc(OCCc3ccc(Cl)c(Cl)c3)cn2)c(N2CCC(C)(C)CC2)c1[C@H](OC(C)(C)C)C(=O)O]

General Procedure F was followed. The experiment afforded the desired compound, (2S)-2-(tert-butoxy)-2-{5-[2-(3,4-dichlorophenyl)ethoxy]-4′-(4,4-dimethylpiperidin-1-yl)-6′-methyl-[2,3′-bipyridine]-5′-yl}acetic acid (8 mg, 25% yield, 98.8% purity). LCMS observed ion=600.2. ¹H NMR (500 MHz, METHANOL-d4) δ 8.36 (s, 1H), 8.15 (s, 1H), 7.55 (s, 2H), 7.50-7.45 (m, 2H), 7.31 (br d, J=8.9 Hz, 1H), 5.81 (br s, 1H), 4.65 (br s, 4H), 4.38 (t, J=6.3 Hz, 2H), 3.16 (t, J=6.1 Hz, 2H), 2.65 (s, 3H), 1.43 (br s, 4H), 1.20 (s, 9H), 0.90 (s, 6H).

### Example 336

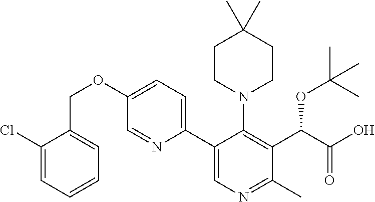 [SMILES: Cc1ncc(-c2ccc(OCc3ccccc3Cl)cn2)c(N2CCC(C)(C)CC2)c1[C@H](OC(C)(C)C)C(=O)O]

General Procedure F was followed. The experiment afforded the desired compound, (2S)-2-(tert-butoxy)-2-{5-[(2-chlorophenyl)methoxy]-4′-(4,4-dimethylpiperidin-1-yl)-6′-methyl-[2,3′-bipyridine]-5′-yl}acetic acid (13.5 mg, 46% yield, 98.5% purity). LCMS observed ion=552.2. ¹H NMR (500 MHz, METHANOL-d4) δ 8.47 (d, J=2.7 Hz, 1H), 8.19 (s, 1H), 7.67-7.60 (m, 2H), 7.53-7.48 (m, 2H), 7.39 (d, J=6.2 Hz, 2H), 5.82 (s, 1H), 5.37 (s, 2H), 2.82 (br s, 2H), 2.91 (br s, 2H), 2.66 (s, 3H), 1.44 (br s, 4H), 1.21 (s, 9H), 0.90 (s, 6H).

### Example 337

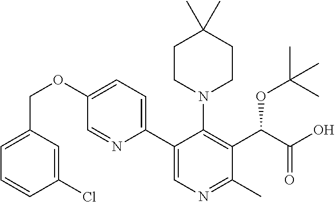 [SMILES: Cc1ncc(-c2ccc(OCc3cccc(Cl)c3)cn2)c(N2CCC(C)(C)CC2)c1[C@H](OC(C)(C)C)C(=O)O]

General Procedure F was followed. The experiment afforded the desired compound, (2S)-2-(tert-butoxy)-2-{5-[(3-chlorophenyl)methoxy]-4′-(4,4-dimethylpiperidin-1-yl)-6′-methyl-[2,3′-bipyridine]-5′-yl}acetic acid (15 mg, 51% yield, 100% purity). LCMS observed ion=552.2. ¹H NMR (500 MHz, METHANOL-d4) δ 8.46 (d, J=2.4 Hz, 1H), 8.18 (s, 1H), 7.63 (dd, J=8.7, 2.9 Hz, 1H), 7.53 (s, 1H), 7.49 (d, J=8.9 Hz, 1H), 7.45 (br d, J=7.6 Hz, 1H), 7.41 (t, J=7.6 Hz, 1H), 7.37 (d, J=7.9 Hz, 1H), 5.81 (s, 1H), 5.30 (s, 2H), 2.94 (br s, 2H), 2.81 (br s, 2H), 2.66 (s, 3H), 1.43 (br s, 4H), 1.21 (s, 9H), 0.89 (s, 6H).

### Example 338

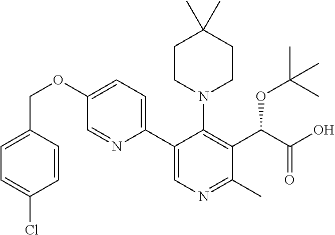 [SMILES: Cc1ncc(-c2ccc(OCc3ccc(Cl)cc3)cn2)c(N2CCC(C)(C)CC2)c1[C@H](OC(C)(C)C)C(=O)O]

General Procedure F was followed. The experiment afforded the desired compound, (2S)-2-(tert-butoxy)-2-{5-[(4-chlorophenyl)methoxy]-4′-(4,4-dimethylpiperidin-1-yl)-6′-methyl-[2,3′-bipyridine]-5′-yl}acetic acid (12.1 mg, 41% yield, 98% purity). LCMS observed ion=552.2. ¹H NMR (500 MHz, DMSO-d6) δ 8.45 (d, J=3.1 Hz, 1H), 8.13 (s, 1H), 7.59 (dd, J=8.7, 2.9 Hz, 1H), 7.54-7.51 (m, 2H), 7.49-7.45 (m, 3H), 5.86 (s, 1H), 5.30-5.23 (m, 2H), 2.49-2.47 (m, 3H), 2.12-1.95 (m, 2H), 1.53 (br s, 1H), 1.29 (br s, 2H), 1.12 (s, 9H), 1.05 (br s, 1H), 0.89 (br s, 3H), 0.72 (br s, 3H).

### Example 339

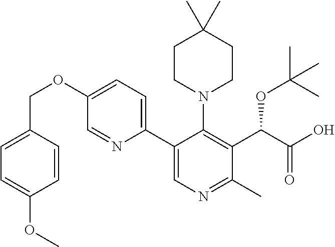 [SMILES: COc1ccc(COc2ccc(-c3cnc(C)c([C@H](OC(C)(C)C)C(=O)O)c3N3CCC(C)(C)CC3)nc2)cc1]

General Procedure F was followed. The experiment afforded the desired compound, (2S)-2-(tert-butoxy)-2-[4′-(4,4-dimethylpiperidin-1-yl)-5-[(4-methoxyphenyl)methoxy]-6′-methyl-[2,3′-bipyridine]-5′-yl]acetic acid (4.1 mg, 14% yield, 95.7% purity). LCMS observed ion=548.3. ¹H NMR (500 MHz, METHANOL-d4) δ 8.48 (d, J=2.4 Hz, 1H), 8.37 (s, 1H), 7.65 (dd, J=8.5, 2.7 Hz, 1H), 7.55 (d, J=8.5 Hz, 1H), 7.42 (br d, J=8.5 Hz, 2H), 6.96 (br d, J=8.5 Hz, 2H), 5.69 (s, 1H), 5.22 (s, 2H), 3.82 (s, 3H), 2.94 (br s, 4H), 2.79 (s, 3H), 1.47 (br s, 4H), 1.24 (s, 9H), 0.95 (s, 6H).

### Example 340

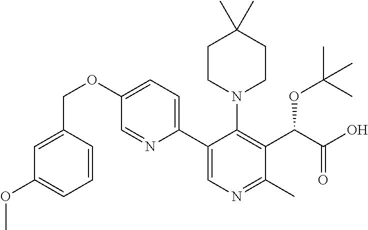 [SMILES: COc1cccc(COc2ccc(-c3cnc(C)c([C@H](OC(C)(C)C)C(=O)O)c3N3CCC(C)(C)CC3)nc2)c1]

General Procedure F was followed. The experiment afforded the desired compound, (2S)-2-(tert-butoxy)-2-[4′-(4,4-dimethylpiperidin-1-yl)-5-[(3-methoxyphenyl)methoxy]-6′-methyl-[2,3′-bipyridine]-5′-yl]acetic acid (16.3 mg, 56% yield, 97.4% purity). LCMS observed ion=548.3. ¹H NMR (500 MHz, METHANOL-d4) δ 8.50 (d, J=2.1 Hz, 1H), 8.36 (s, 1H), 7.65 (dd, J=8.5, 2.7 Hz, 1H), 7.55 (d, J=8.9 Hz, 1H), 7.32 (t, J=8.1 Hz, 1H), 7.05 (s, 1H), 7.06 (d, J=7.5 Hz, 1H), 6.92 (br d, J=8.9 Hz, 1H), 5.70 (s, 1H), 5.29 (s, 2H), 3.82 (s, 3H), 2.93 (br s, 4H), 2.78 (s, 3H), 1.47 (br s, 4H), 1.24 (s, 9H), 0.94 (s, 6H).

### Example 341

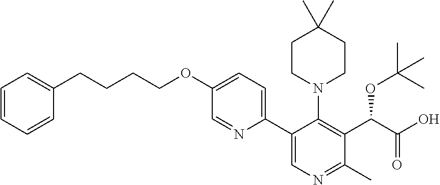 [SMILES: Cc1ncc(-c2ccc(OCCCCc3ccccc3)cn2)c(N2CCC(C)(C)CC2)c1[C@H](OC(C)(C)C)C(=O)O]

General Procedure F was followed. The experiment afforded the desired compound, (2S)-2-(tert-butoxy)-2-[4′-(4,4-dimethylpiperidin-1-yl)-6′-methyl-5-(4-phenylbutoxy)-[2,3′-bipyridine]-5′-yl]acetic acid (11.8 mg, 39% yield, 94.5% purity). LCMS observed ion=560.3. ¹H NMR (500 MHz, METHANOL-d4) δ 8.50 (d, J=2.1 Hz, 1H), 8.36 (s, 1H), 7.65 (dd, J=8.5, 2.7 Hz, 1H), 7.55 (d, J=8.9 Hz, 1H), 7.32 (t, J=8.1 Hz, 1H), 7.05 (s, 1H), 7.06 (d, J=7.5 Hz, 1H), 6.92 (br d, J=8.9 Hz, 1H), 5.70 (s, 1H), 5.29 (s, 2H), 3.82 (s, 3H), 2.93 (br s, 4H), 2.78 (s, 3H), 1.47 (br s, 4H), 1.24 (s, 9H), 0.94 (s, 6H).

### Example 342

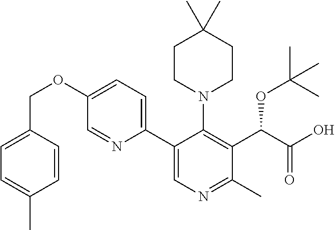 [SMILES: Cc1ccc(COc2ccc(-c3cnc(C)c([C@H](OC(C)(C)C)C(=O)O)c3N3CCC(C)(C)CC3)nc2)cc1]

General Procedure F was followed. The experiment afforded the desired compound, (2S)-2-(tert-butoxy)-2-[4′-(4,4-dimethylpiperidin-1-yl)-6′-methyl-5-[(4-methylphenyl)methoxy]-[2,3′-bipyridine]-5′-yl]acetic acid (12.9 mg, 45% yield, 100% purity). LCMS observed ion=532.3. ¹H NMR (500 MHz, METHANOL-d4) δ 8.42 (br s, 1H), 8.17 (br s, 1H), 7.61 (br d, J=8.2 Hz, 1H), 7.46 (d, J=8.9 Hz, 1H), 7.38 (br d, J=7.9 Hz, 2H), 7.23 (br d, J=7.6 Hz, 2H), 5.80 (br s, 1H), 5.24 (s, 2H), 2.79 (br s, 2H), 2.91 (br s, 2H), 2.66 (s, 3H), 2.36 (s, 3H), 1.42 (br s, 4H), 1.21 (s, 9H), 0.89 (s, 6H).

### Example 343

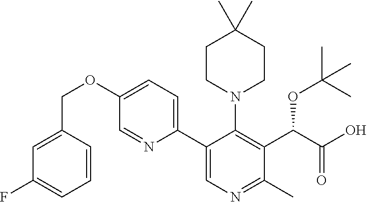 [SMILES: Cc1ncc(-c2ccc(OCc3cccc(F)c3)cn2)c(N2CCC(C)(C)CC2)c1[C@H](OC(C)(C)C)C(=O)O]

General Procedure F was followed. The experiment afforded the desired compound, (2S)-2-(tert-butoxy)-2-[4′-(4,4-dimethylpiperidin-1-yl)-5-[(3-fluorophenyl)methoxy]-6′-methyl-[2,3′-bipyridine]-5′-yl]acetic acid (12.8 mg, 45% yield, 100% purity). LCMS observed ion=536.3. ¹H NMR (500 MHz, METHANOL-d4) δ 8.46 (d, J=2.4 Hz, 1H), 8.18 (s, 1H), 7.63 (d, J=8.3 Hz, 1H), 7.50-7.41 (m, 2H), 7.33 (br d, J=7.9 Hz, 1H), 7.26 (br d, J=10.1 Hz, 1H), 7.09 (br t, J=8.4 Hz, 1H), 5.81 (s, 1H), 5.31 (s, 2H), 2.81 (br s, 2H), 2.94 (br s, 2H), 2.66 (s, 3H), 1.43 (br s, 4H), 1.21 (s, 9H), 0.89 (s, 6H).

### Example 344

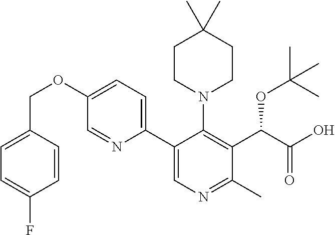 [SMILES: Cc1ncc(-c2ccc(OCc3ccc(F)cc3)cn2)c(N2CCC(C)(C)CC2)c1[C@H](OC(C)(C)C)C(=O)O]

General Procedure F was followed. The experiment afforded the desired compound, (2S)-2-(tert-butoxy)-2-[4′-(4,4-dimethylpiperidin-1-yl)-5-[(4-fluorophenyl)methoxy]-6′-methyl-[2,3′-bipyridine]-5′-yl]acetic acid (8.6 mg, 30% yield, 100% purity). LCMS observed ion=536.3. ¹H NMR (500 MHz, METHANOL-d4) δ 8.44 (br s, 1H), 8.18 (s, 1H), 7.63 (dd, J=8.5, 2.7 Hz, 1H), 7.54 (t, J=6.4 Hz, 2H), 7.48 (d, J=8.9 Hz, 1H), 7.17-7.13 (m, 2H), 5.81 (br s, 1H), 5.26 (s, 2H), 2.93 (br s, 2H), 2.79 (br s, 2H), 2.66 (s, 3H), 1.43 (br s, 4H), 1.21 (s, 9H), 0.89 (s, 6H).

### Example 345

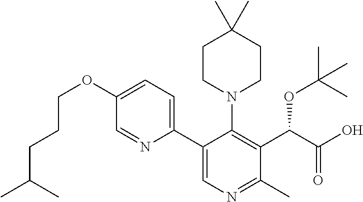 [SMILES: Cc1ncc(-c2ccc(OCCCC(C)C)cn2)c(N2CCC(C)(C)CC2)c1[C@H](OC(C)(C)C)C(=O)O]

General Procedure F was followed. The experiment afforded the desired compound, (2S)-2-(tert-butoxy)-2-[4′-(4,4-dimethylpiperidin-1-yl)-6′-methyl-5-[(4-methylpentyl)oxy]-[2,3′-bipyridine]-5′-yl]acetic acid (11 mg, 40% yield, 100% purity). LCMS observed ion=512.4. ¹H NMR (500 MHz, METHANOL-d4) δ 8.37 (s, 1H), 8.19 (s, 1H), 7.56 (br d, J=8.9 Hz, 1H), 7.48 (d, J=8.5 Hz, 1H), 5.81 (s, 1H), 4.14 (t, J=6.4 Hz, 2H), 2.97 (br s, 2H), 2.84 (br s, 2H), 2.67 (s, 3H), 1.90-1.84 (m, 2H), 1.70-1.61 (m, 1H), 1.50-1.38 (m, 6H), 1.21 (s, 9H), 0.97 (d, J=6.7 Hz, 6H), 0.92 (s, 6H).

### Example 346

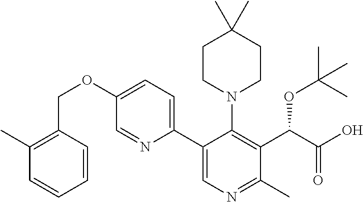 [SMILES: Cc1ccccc1COc1ccc(-c2cnc(C)c([C@H](OC(C)(C)C)C(=O)O)c2N2CCC(C)(C)CC2)nc1]

General Procedure F was followed. The experiment afforded the desired compound, (2S)-2-(tert-butoxy)-2-[4′-(4,4-dimethylpiperidin-1-yl)-6′-methyl-5-[(2-methylphenyl)methoxy]-[2,3′-bipyridine]-5′-yl]acetic acid (13.8 mg, 49% yield, 100% purity). LCMS observed ion=532.3. ¹H NMR (500 MHz, METHANOL-d4) δ 8.45 (d, J=2.1 Hz, 1H), 8.19 (s, 1H), 7.65 (d, J=8.2 Hz, 1H), 7.49 (d, J=8.5 Hz, 1H), 7.44 (br d, J=7.3 Hz, 1H), 7.29-7.20 (m, 3H), 5.82 (s, 1H), 5.28 (s, 2H), 2.97 (br s, 2H), 2.83 (br s, 2H), 2.66 (s, 3H), 2.43 (s, 3H), 1.44 (br s, 4H), 1.21 (s, 9H), 0.90 (s, 6H).

### Example 347

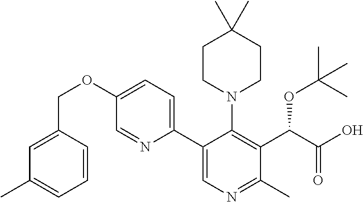 [SMILES: Cc1cccc(COc2ccc(-c3cnc(C)c([C@H](OC(C)(C)C)C(=O)O)c3N3CCC(C)(C)CC3)nc2)c1]

General Procedure F was followed. The experiment afforded the desired compound, (2S)-2-(tert-butoxy)-2-[4′-(4,4-dimethylpiperidin-1-yl)-6′-methyl-5-[(3-methylphenyl)methoxy]-[2,3′-bipyridine]-5′-yl]acetic acid (12 mg, 42% yield, 100% purity). LCMS observed ion=532.3. ¹H NMR (500 MHz, METHANOL-d4) δ 8.44 (d, J=2.4 Hz, 1H), 8.18 (br s, 1H), 7.62 (dd, J=8.5, 2.7 Hz, 1H), 7.47 (d, J=8.9 Hz, 1H), 7.33-7.27 (m, 3H), 7.18 (br d, J=5.5 Hz, 1H), 5.80 (br s, 1H), 5.25 (s, 2H), 2.94 (br s, 2H), 2.81 (br s, 2H), 2.66 (s, 3H), 2.38 (s, 3H), 1.43 (br s, 4H), 1.21 (s, 9H), 0.89 (s, 6H).

### Example 348

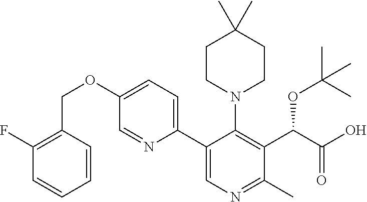 [SMILES: Cc1ncc(-c2ccc(OCc3ccccc3F)cn2)c(N2CCC(C)(C)CC2)c1[C@H](OC(C)(C)C)C(=O)O]

General Procedure F was followed. The experiment afforded the desired compound, (2S)-2-(tert-butoxy)-2-[4′-(4,4-dimethylpiperidin-1-yl)-5-[(2-fluorophenyl)methoxy]-6′-methyl-[2,3′-bipyridine]-5′-yl]acetic acid (11.7 mg, 41% yield, 100% purity). LCMS observed ion=536.3. ¹H NMR (500 MHz, METHANOL-d4) δ 8.46 (d, J=2.4 Hz, 1H), 8.19 (s, 1H), 7.66 (dd, J=8.5, 2.7 Hz, 1H), 7.58 (t, J=7.5 Hz, 1H), 7.50 (d, J=8.5 Hz, 1H), 7.45-7.40 (m, 1H), 7.24 (t, J=7.5 Hz, 1H), 7.19 (t, J=9.3 Hz, 1H), 5.82 (br s, 1H), 5.34 (s, 2H), 2.93 (br s, 2H), 2.82 (br s, 2H), 2.66 (s, 3H), 1.44 (br s, 4H), 1.21 (s, 9H), 0.90 (s, 6H).

### Example 349

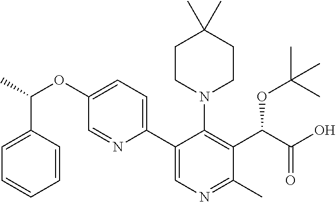 [SMILES: Cc1ncc(-c2ccc(O[C@@H](C)c3ccccc3)cn2)c(N2CCC(C)(C)CC2)c1[C@H](OC(C)(C)C)C(=O)O]

General Procedure F was followed. The experiment afforded the desired compound, (2S)-2-(tert-butoxy)-2-[4′-(4,4-dimethylpiperidin-1-yl)-6′-methyl-5-[(1S)-1-phenylethoxy]-[2,3′-bipyridine]-5′-yl]acetic acid (10.3 mg, 36% yield, 100% purity). LCMS observed ion=532.2. ¹H NMR (500 MHz, METHANOL-d4) δ 8.34 (d, J=2.1 Hz, 1H), 8.13 (s, 1H), 7.49-7.43 (m, 3H), 7.38-7.34 (m, 3H), 7.30-7.26 (m, 1H), 5.78 (br s, 1H), 5.61 (q, J=6.3 Hz, 1H), 2.88 (br s, 2H), 2.74 (br s, 2H), 2.64 (s, 3H), 1.71 (d, J=6.4 Hz, 3H), 1.39 (br s, 4H), 1.19 (s, 9H), 0.90 (s, 6H).

### Example 350

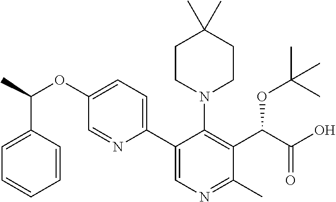 [SMILES: Cc1ncc(-c2ccc(O[C@H](C)c3ccccc3)cn2)c(N2CCC(C)(C)CC2)c1[C@H](OC(C)(C)C)C(=O)O]

General Procedure F was followed. The experiment afforded the desired compound, (2S)-2-(tert-butoxy)-2-[4′-(4,4-dimethylpiperidin-1-yl)-6′-methyl-5-[(1R)-1-phenylethoxy]-[2,3′-bipyridine]-5′-yl]acetic acid (16.8 mg, 59% yield, 100% purity). LCMS observed ion=532.2. ¹H NMR (500 MHz, METHANOL-d4) δ 8.29 (br s, 1H), 8.04 (s, 1H), 7.44 (br d, J=7.0 Hz, 3H), 7.38-7.26 (m, 4H), 5.70 (s, 1H), 5.59 (br d, J=5.8 Hz, 1H), 2.62 (s, 3H), 2.77-2.52 (m, 4H), 1.70 (br d, J=6.4 Hz, 3H), 1.33 (br s, 4H), 1.16 (s, 9H), 0.76 (br s, 6H).

### Example 351

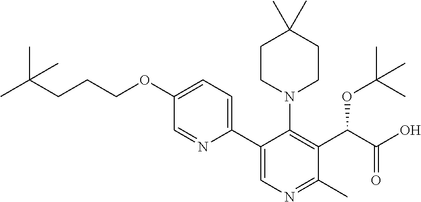 [SMILES: Cc1ncc(-c2ccc(OCCCC(C)(C)C)cn2)c(N2CCC(C)(C)CC2)c1[C@H](OC(C)(C)C)C(=O)O]

General procedure B was followed. The experiment afforded the desired compound, (2S)-2-(tert-butoxy)-2-{5-[(4,4-dimethylpentyl)oxy]-4′-(4,4-dimethylpiperidin-1-yl)-6′-methyl-[2,3′-bipyridine]-5′-yl}acetic acid (12.9 mg, 46% yield, 100% purity). LCMS observed ion=526.4. ¹H NMR (500 MHz, METHANOL-d4) δ 8.38 (br s, 1H), 8.21 (s, 1H), 7.57 (br d, J=8.5 Hz, 1H), 7.49 (d, J=8.5 Hz, 1H), 5.80 (s, 1H), 4.14 (br t, J=6.3 Hz, 2H), 2.99 (br s, 2H), 2.84 (br s, 2H), 2.68 (s, 3H), 1.88-1.81 (m, 2H), 1.48-1.38 (m, 6H), 1.22 (s, 9H), 0.98-0.96 (m, 9H), 0.92 (s, 6H).

### Example 352

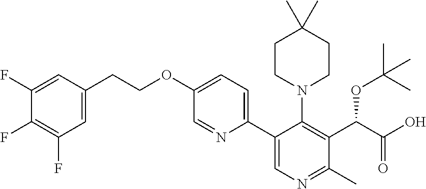 [SMILES: Cc1ncc(-c2ccc(OCCc3cc(F)c(F)c(F)c3)cn2)c(N2CCC(C)(C)CC2)c1[C@H](OC(C)(C)C)C(=O)O]

General Procedure F was followed. The experiment afforded the desired compound, (2S)-2-(tert-butoxy)-2-[4′-(4,4-dimethylpiperidin-1-yl)-6′-methyl-5-[2-(3,4,5-trifluorophenyl)ethoxy]-[2,3′-bipyridine]-5′-yl]acetic acid (5.5 mg, 17% yield, 99.3% purity). LCMS observed ion=586.2. ¹H NMR (500 MHz, METHANOL-d4) δ 8.38 (d, J=2.4 Hz, 1H), 8.19 (s, 1H), 7.57 (d, J=8.3 Hz, 1H), 7.48 (d, J=8.5 Hz, 1H), 7.19-7.13 (m, 2H), 5.80 (s, 1H), 4.38 (t, J=6.3 Hz, 2H), 3.16 (brt, J=6.1 Hz, 2H), 2.98 (br s, 2H), 2.83 (br s, 2H), 2.67 (s, 3H), 1.44 (br s, 4H), 1.21 (s, 9H), 0.91 (s, 6H).

### Example 353

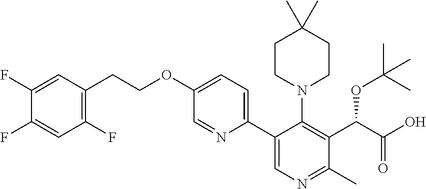 [SMILES: Cc1ncc(-c2ccc(OCCc3cc(F)c(F)cc3F)cn2)c(N2CCC(C)(C)CC2)c1[C@H](OC(C)(C)C)C(=O)O]

General Procedure F was followed. The experiment afforded the desired compound, (2S)-2-(tert-butoxy)-2-[4′-(4,4-dimethylpiperidin-1-yl)-6′-methyl-5-[2-(2,4,5-trifluorophenyl)ethoxy]-[2,3′-bipyridine]-5′-yl]acetic acid (13.7 mg, 44% yield, 100% purity). LCMS observed ion=586.3. ¹H NMR (500 MHz, METHANOL-d4) δ 8.33 (d, J=2.4 Hz, 1H), 8.10 (s, 1H), 7.54 (d, J=8.3 Hz, 1H), 7.45 (d, J=8.5 Hz, 1H), 7.43-7.36 (m, 1H), 7.22-7.14 (m, 1H), 5.75 (s, 1H), 4.38 (t, J=6.4 Hz, 2H), 3.18 (br t, J=6.1 Hz, 2H), 2.77 (br s, 2H), 2.64 (s, 3H), 1.42 (br s, 4H), 1.18 (s, 9H), 0.89 (s, 6H).

### Example 354

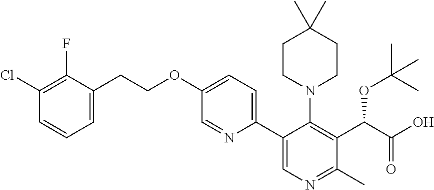 [SMILES: Cc1ncc(-c2ccc(OCCc3cccc(Cl)c3F)cn2)c(N2CCC(C)(C)CC2)c1[C@H](OC(C)(C)C)C(=O)O]

General Procedure F was followed. The experiment afforded the desired compound, (2S)-2-(tert-butoxy)-2-{5-[2-(3-chloro-2-fluorophenyl)ethoxy]-4′-(4,4-dimethylpiperidin-1-yl)-6′-methyl-[2,3′-bipyridine]-5′-yl}acetic acid (12.8 mg, 41% yield, 99.3% purity). LCMS observed ion=584.2. ¹H NMR (500 MHz, METHANOL-d4) δ 8.42 (s, 1H), 8.36 (s, 1H), 7.61-7.54 (m, 2H), 7.41-7.36 (m, 2H), 7.16 (t, J=7.6 Hz, 1H), 5.70 (s, 1H), 4.42 (t, J=6.3 Hz, 2H), 3.26 (br t, J=6.1 Hz, 2H), 3.11-2.87 (m, 4H), 2.79 (s, 3H), 1.48 (br s, 4H), 1.24 (s, 9H), 0.96 (s, 6H).

### Example 355

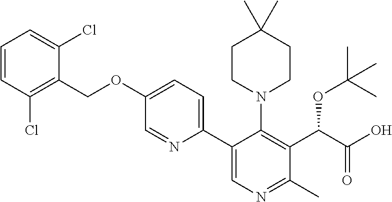 [SMILES: Cc1ncc(-c2ccc(OCc3c(Cl)cccc3Cl)cn2)c(N2CCC(C)(C)CC2)c1[C@H](OC(C)(C)C)C(=O)O]

General Procedure F was followed. The experiment afforded the desired compound, (2S)-2-(tert-butoxy)-2-{5-[(2,6-dichlorophenyl)methoxy]-4′-(4,4-dimethylpiperidin-1-yl)-6′-methyl-[2,3′-bipyridine]-5′-yl}acetic acid (16.8 mg, 54% yield, 100% purity). LCMS observed ion=586.2. ¹H NMR (500 MHz, DMSO-d6) δ 8.49 (d, J=2.7 Hz, 1H), 8.16 (s, 1H), 7.69 (dd, J=8.5, 2.7 Hz, 1H), 7.58 (d, J=7.6 Hz, 2H), 7.52-7.47 (m, 2H), 5.87 (s, 1H), 5.38 (s, 2H), 2.48 (br s, 3H), 2.17-1.90 (m, 2H), 1.54 (br s, 1H), 1.30 (br s, 2H), 1.12 (s, 9H), 1.10-1.03 (m, 1H), 0.88 (br s, 3H), 0.76 (br s, 3H).

### Example 356

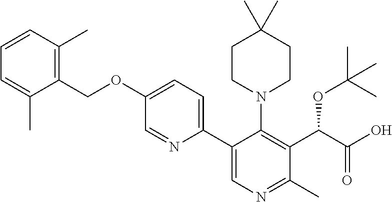 [SMILES: Cc1cccc(C)c1COc1ccc(-c2cnc(C)c([C@H](OC(C)(C)C)C(=O)O)c2N2CCC(C)(C)CC2)nc1]

General Procedure F was followed. The experiment afforded the desired compound, (2S)-2-(tert-butoxy)-2-{5-[(2,6-dimethylphenyl)methoxy]-4′-(4,4-dimethylpiperidin-1-yl)-6′-methyl-[2,3′-bipyridine]-5′-yl}acetic acid (6 mg, 20% yield, 99.3% purity). LCMS observed ion=546.3. ¹H NMR (500 MHz, METHANOL-d4) δ 8.47 (s, 1H), 8.21 (br s, 1H), 7.71 (d, J=8.1 Hz, 1H), 7.53 (d, J=8.5 Hz, 1H), 7.21-7.17 (m, 1H), 7.11 (d, J=7.3 Hz, 2H), 5.81 (br s, 1H), 5.29 (s, 2H), 3.01 (br s, 2H), 2.85 (br s, 2H), 2.68 (s, 3H), 2.42 (s, 6H), 1.46 (br s, 4H), 1.22 (s, 9H), 0.93 (s, 6H).

### Example 357

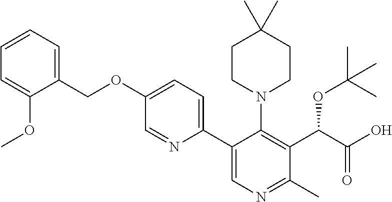 [SMILES: COc1ccccc1COc1ccc(-c2cnc(C)c([C@H](OC(C)(C)C)C(=O)O)c2N2CCC(C)(C)CC2)nc1]

General Procedure F was followed. The experiment afforded the desired compound, (2S)-2-(tert-butoxy)-2-[4′-(4,4-dimethylpiperidin-1-yl)-5-[(2-methoxyphenyl)methoxy]-6′-methyl-[2,3′-bipyridine]-5′-yl]acetic acid (12.3 mg, 42% yield, 100% purity). LCMS observed ion=548.3. ¹H NMR (500 MHz, METHANOL-d4) δ 8.42 (br s, 1H), 8.19 (s, 1H), 7.61 (dd, J=8.5, 2.1 Hz, 1H), 7.48 (d, J=8.1 Hz, 1H), 7.43 (d, J=7.3 Hz, 1H), 7.36 (t, J=7.6 Hz, 1H), 7.06 (d, J=7.9 Hz, 1H), 6.98 (t, J=7.5 Hz, 1H), 5.80 (br s, 1H), 5.29 (s, 2H), 3.91 (s, 3H), 3.00 (br s, 2H), 2.82 (br s, 2H), 2.67 (s, 3H), 1.44 (br s, 4H), 1.21 (s, 9H), 0.90 (s, 6H).

### Example 358

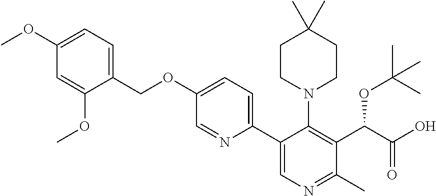 [SMILES: COc1ccc(COc2ccc(-c3cnc(C)c([C@H](OC(C)(C)C)C(=O)O)c3N3CCC(C)(C)CC3)nc2)c(OC)c1]

General Procedure F was followed. The experiment afforded the desired compound, (2S)-2-(tert-butoxy)-2-{5-[(2,4-dimethoxyphenyl)methoxy]-4′-(4,4-dimethylpiperidin-1-yl)-6′-methyl-[2,3′-bipyridine]-5′-yl}acetic acid (7.4 mg, 24% yield, 98.5% purity). LCMS observed ion=578.3. ¹H NMR (500 MHz, METHANOL-d4) δ 8.40 (d, J=2.7 Hz, 1H), 8.19 (s, 1H), 7.61 (dd, J=8.2, 2.7 Hz, 1H), 7.47 (d, J=8.5 Hz, 1H), 7.33 (d, J=8.5 Hz, 1H), 6.60 (s, 1H), 6.55 (br d, J=8.5 Hz, 1H), 5.79 (s, 1H), 5.21-5.19 (m, 2H), 3.88 (s, 3H), 3.83 (s, 3H), 2.95 (br s, 2H), 2.81 (br s, 2H), 2.67 (s, 3H), 1.44 (br s, 4H), 1.21 (s, 9H), 0.90 (s, 6H).

### Example 359

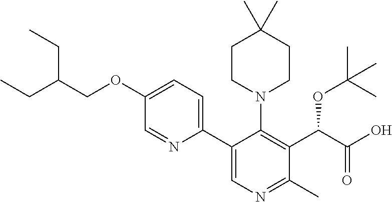 [SMILES: CCC(CC)COc1ccc(-c2cnc(C)c([C@H](OC(C)(C)C)C(=O)O)c2N2CCC(C)(C)CC2)nc1]

General procedure B was followed. The experiment afforded the desired compound, (2S)-2-(tert-butoxy)-2-[4′-(4,4-dimethylpiperidin-1-yl)-5-(2-ethylbutoxy)-6′-methyl-[2,3′-bipyridine]-5′-yl]acetic acid (14.9 mg, 54% yield, 100% purity). LCMS observed ion=512.3. ¹H NMR (500 MHz, METHANOL-d4) δ 8.37 (d, J=2.4 Hz, 1H), 8.17 (s, 1H), 7.57 (dd, J=8.4, 2.6 Hz, 1H), 7.47 (d, J=8.5 Hz, 1H), 5.80 (s, 1H), 4.07 (d, J=5.8 Hz, 2H), 2.98 (br s, 2H), 2.82 (br s, 2H), 2.66 (s, 3H), 1.77-1.71 (m, 1H), 1.61-1.49 (m, 4H), 1.44 (br s, 4H), 1.21 (s, 9H), 1.00 (t, J=7.3 Hz, 6H), 0.91 (s, 6H).

### Example 360

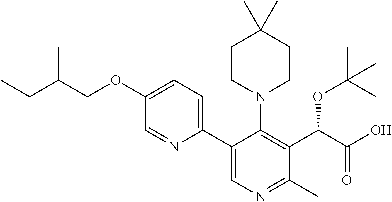 [SMILES: CCC(C)COc1ccc(-c2cnc(C)c([C@H](OC(C)(C)C)C(=O)O)c2N2CCC(C)(C)CC2)nc1]

General Procedure F was followed. The experiment afforded the desired compound, (2S)-2-(tert-butoxy)-2-[4′-(4,4-dimethylpiperidin-1-yl)-6′-methyl-5-(2-methylbutoxy)-[2,3′-bipyridine]-5′-yl]acetic acid (13.3 mg, 50% yield, 100% purity). LCMS observed ion=498.3. ¹H NMR (500 MHz, METHANOL-d4) δ 8.39 (s, 1H), 8.21 (br s, 1H), 7.57 (d, J=8.1 Hz, 1H), 7.49 (d, J=8.1 Hz, 1H), 5.78 (br s, 1H), 4.02 (br d, J=6.1 Hz, 1H), 3.98-3.93 (m, 1H), 3.02 (br s, 2H), 2.85 (br s, 2H), 2.69 (s, 3H), 1.97-1.90 (m, 1H), 1.69-1.60 (m, 1H), 1.46 (br s, 4H), 1.39-1.32 (m, 1H), 1.22 (s, 9H), 1.09 (d, J=6.7 Hz, 3H), 1.01 (t, J=7.5 Hz, 3H), 0.92 (s, 6H).

### Example 361

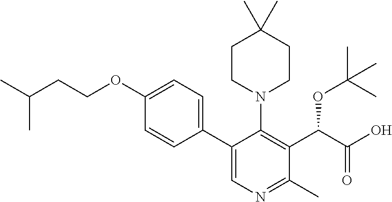 [SMILES: Cc1ncc(-c2ccc(OCCC(C)C)cc2)c(N2CCC(C)(C)CC2)c1[C@H](OC(C)(C)C)C(=O)O]

General Procedure G was followed. The experiment afforded the desired compound, (2S)-2-(tert-butoxy)-2-[4-(4,4-dimethylpiperidin-1-yl)-2-methyl-5-[4-(3-methylbutoxy)phenyl]pyridin-3-yl]acetic acid (8.3 mg, 31% yield, 97.7% purity). LCMS observed ion=497.3.

### Example 362

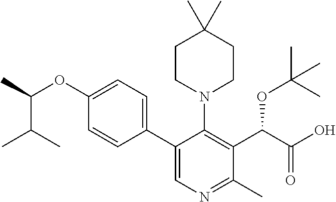 [SMILES: Cc1ncc(-c2ccc(O[C@H](C)C(C)C)cc2)c(N2CCC(C)(C)CC2)c1[C@H](OC(C)(C)C)C(=O)O]

General Procedure G was followed. The experiment afforded the desired compound, (2S)-2-(tert-butoxy)-2-[4-(4,4-dimethylpiperidin-1-yl)-2-methyl-5-(4-{[(2R)-3-methylbutan-2-yl]oxy}phenyl)pyridin-3-yl]acetic acid (4.6 mg, 17% yield, 98.8% purity). LCMS observed ion=497.3. ¹H NMR (500 MHz, METHANOL-d4) δ 8.09 (br s, 1H), 7.26 (br d, J=7.9 Hz, 2H), 7.07 (br d, J=8.5 Hz, 2H), 5.68 (br s, 1H), 4.33 (br t, J=6.0 Hz, 1H), 2.78 (br s, 2H), 2.68 (s, 3H), 2.00-1.92 (m, 1H), 1.44 (br s, 4H), 1.28 (d, J=6.4 Hz, 3H), 1.20 (s, 9H), 1.05 (d, J=6.7 Hz, 3H), 1.02 (d, J=6.7 Hz, 3H), 0.91 (s, 6H).

### Example 363

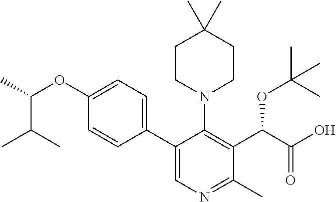 [SMILES: Cc1ncc(-c2ccc(O[C@@H](C)C(C)C)cc2)c(N2CCC(C)(C)CC2)c1[C@H](OC(C)(C)C)C(=O)O]

General Procedure G was followed. The experiment afforded the desired compound, (2S)-2-(tert-butoxy)-2-[4-(4,4-dimethylpiperidin-1-yl)-2-methyl-5-(4-{[(2S)-3-methylbutan-2-yl]oxy}phenyl)pyridin-3-yl]acetic acid (6.4 mg, 24% yield, 99.4% purity). LCMS observed ion=497.3. ¹H NMR (500 MHz, METHANOL-d4) δ 8.09 (br s, 1H), 7.26 (br d, J=7.9 Hz, 2H), 7.07 (br d, J=8.5 Hz, 2H), 5.68 (br s, 1H), 4.33 (br t, J=6.0 Hz, 1H), 2.78 (br s, 2H), 2.68 (s, 3H), 2.00-1.92 (m, 1H), 1.44 (br s, 4H), 1.28 (d, J=6.4 Hz, 3H), 1.20 (s, 9H), 1.05 (d, J=6.7 Hz, 3H), 1.02 (d, J=6.7 Hz, 3H), 0.91 (s, 6H).

### Example 364

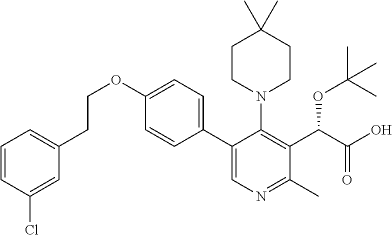 [SMILES: Cc1ncc(-c2ccc(OCCc3cccc(Cl)c3)cc2)c(N2CCC(C)(C)CC2)c1[C@H](OC(C)(C)C)C(=O)O]

General Procedure G was followed. The experiment afforded the desired compound, (2S)-2-(tert-butoxy)-2-(5-{4-[2-(3-chlorophenyl)ethoxy]phenyl}-4-(4,4-dimethylpiperidin-1-yl)-2-methylpyridin-3-yl)acetic acid (8.2 mg, 27% yield, 98.3% purity). LCMS observed ion=565.3. ¹H NMR (500 MHz, METHANOL-d4) δ 8.12 (br s, 1H), 7.37 (s, 1H), 7.33-7.24 (m, 5H), 7.09 (br d, J=8.2 Hz, 2H), 5.67 (br s, 1H), 4.29 (br t, J=6.4 Hz, 2H), 3.13 (br t, J=6.4 Hz, 2H), 3.17 (br s, 2H), 2.79 (br s, 2H), 2.71 (s, 3H), 1.45 (br s, 4H), 1.21 (s, 9H), 0.92 (s, 6H).

### Example 365

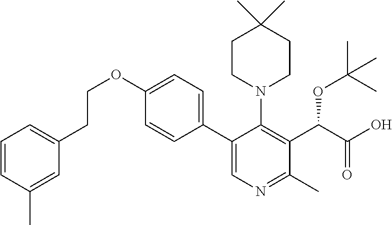 [SMILES: Cc1cccc(CCOc2ccc(-c3cnc(C)c([C@H](OC(C)(C)C)C(=O)O)c3N3CCC(C)(C)CC3)cc2)c1]

General Procedure G was followed. The experiment afforded the desired compound, (2S)-2-(tert-butoxy)-2-[4-(4,4-dimethylpiperidin-1-yl)-2-methyl-5-{4-[2-(3-methylphenyl)ethoxy]phenyl}pyridin-3-yl]acetic acid (9.1 mg, 31% yield, 98.4% purity). LCMS observed ion=545.3. ¹H NMR (500 MHz, METHANOL-d4) δ 8.10 (s, 1H), 7.27 (br d, J=7.6 Hz, 2H), 7.22-7.15 (m, 2H), 7.13-7.05 (m, 4H), 5.67 (br s, 1H), 4.26 (t, J=6.9 Hz, 2H), 3.19 (br s, 2H), 3.08 (br t, J=6.9 Hz, 2H), 2.80 (br s, 2H), 2.70 (s, 3H), 2.34 (s, 3H), 1.45 (br s, 4H), 1.21 (s, 9H), 0.92 (s, 6H).

### Example 366

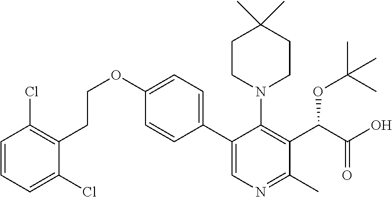 [SMILES: Cc1ncc(-c2ccc(OCCc3c(Cl)cccc3Cl)cc2)c(N2CCC(C)(C)CC2)c1[C@H](OC(C)(C)C)C(=O)O]

General Procedure G was followed. The experiment afforded the desired compound, (2S)-2-(tert-butoxy)-2-(5-{4-[2-(2,6-dichlorophenyl)ethoxy]phenyl}-4-(4,4-dimethylpiperidin-1-yl)-2-methylpyridin-3-yl)acetic acid (5.7 mg, 17% yield, 97.9% purity). LCMS observed ion=599.2. ¹H NMR (500 MHz, METHANOL-d4) δ 8.06 (s, 1H), 7.43 (d, J=8.2 Hz, 2H), 7.29-7.24 (m, 3H), 7.08 (br d, J=8.5 Hz, 2H), 5.67 (s, 1H), 4.30 (t, J=7.2 Hz, 2H), 3.51 (br t, J=7.2 Hz, 2H), 2.75 (br s, 2H), 2.67 (s, 3H), 1.44 (br s, 4H), 1.20 (s, 9H), 0.91 (s, 6H).

### Example 367

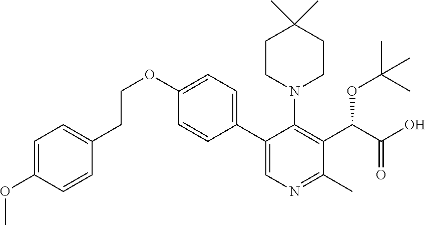 [SMILES: COc1ccc(CCOc2ccc(-c3cnc(C)c([C@H](OC(C)(C)C)C(=O)O)c3N3CCC(C)(C)CC3)cc2)cc1]

General Procedure G was followed. The experiment afforded the desired compound, (2S)-2-(tert-butoxy)-2-[4-(4,4-dimethylpiperidin-1-yl)-5-{4-[2-(4-methoxyphenyl)ethoxy]phenyl}-2-methylpyridin-3-yl]acetic acid (9.7 mg, 32% yield, 100% purity). LCMS observed ion=561.3. ¹H NMR (500 MHz, METHANOL-d4) δ 8.07 (s, 1H), 7.29-7.23 (m, 4H), 7.07 (br d, J=8.5 Hz, 2H), 6.88 (br d, J=8.2 Hz, 2H), 5.67 (s, 1H), 4.23 (t, J=6.9 Hz, 2H), 3.79 (s, 3H), 3.06 (br t, J=6.9 Hz, 2H), 2.77 (br s, 2H), 2.68 (s, 3H), 1.44 (br s, 4H), 1.20 (s, 9H), 0.91 (s, 6H).

### Example 368

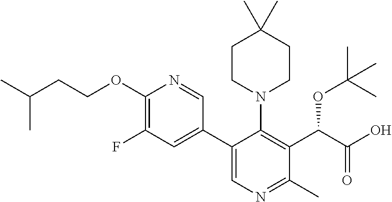 [SMILES: Cc1ncc(-c2cnc(OCCC(C)C)c(F)c2)c(N2CCC(C)(C)CC2)c1[C@H](OC(C)(C)C)C(=O)O]

General Procedure H was followed. The experiment afforded the desired compound, (2S)-2-(tert-butoxy)-2-[4-(4,4-dimethylpiperidin-1-yl)-5′-fluoro-6-methyl-6′-(3-methylbutoxy)-[3,3′-bipyridine]-5-yl]acetic acid (26.6 mg, 51% yield, 515.67% purity). LCMS observed ion=516.3. ¹H NMR (500 MHz, METHANOL-d4) δ 8.15 (br s, 1H), 7.92 (s, 1H), 7.57 (br d, J=11.0 Hz, 1H), 5.78 (br s, 1H), 4.56-4.48 (m, 2H), 3.11 (br s, 2H), 2.79 (br s, 2H), 2.66 (br s, 3H), 1.90-1.82 (m, 1H), 1.75 (q, J=6.5 Hz, 2H), 1.46 (br s, 4H), 1.21 (s, 9H), 1.02 (d, J=6.7 Hz, 6H), 0.93 (s, 6H).

### Example 369

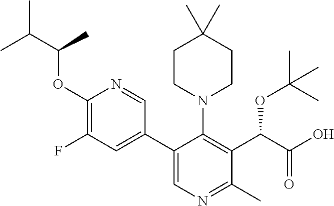 [SMILES: Cc1ncc(-c2cnc(O[C@H](C)C(C)C)c(F)c2)c(N2CCC(C)(C)CC2)c1[C@H](OC(C)(C)C)C(=O)O]

General Procedure H was followed. The experiment afforded the desired compound, (2S)-2-(tert-butoxy)-2-[4-(4,4-dimethylpiperidin-1-yl)-5′-fluoro-6-methyl-6′-{[(2R)-3-methylbutan-2-yl]oxy}-[3,3′-bipyridine]-5-yl]acetic acid (14.8 mg, 28% yield, 515.67% purity). LCMS observed ion=516.3. ¹H NMR (500 MHz, METHANOL-d4) δ 8.15 (s, 1H), 7.91 (s, 1H), 7.56 (br d, J=10.7 Hz, 1H), 5.79 (br s, 1H), 5.24 (brt, J=6.0 Hz, 1H), 3.12 (br s, 2H), 2.79 (br s, 2H), 2.66 (s, 3H), 2.06-1.99 (m, 1H), 1.45 (br s, 4H), 1.35 (d, J=6.1 Hz, 3H), 1.21 (s, 9H), 1.03 (t, J=7.2 Hz, 6H), 0.92 (s, 6H).

### Example 370

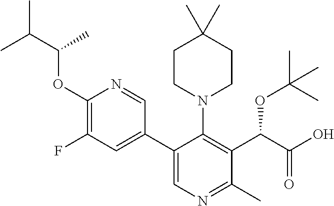 [SMILES: Cc1ncc(-c2cnc(O[C@@H](C)C(C)C)c(F)c2)c(N2CCC(C)(C)CC2)c1[C@H](OC(C)(C)C)C(=O)O]

General Procedure H was followed. The experiment afforded the desired compound, (2S)-2-(tert-butoxy)-2-[4-(4,4-dimethylpiperidin-1-yl)-5′-fluoro-6-methyl-6′-{[(2S)-3-methylbutan-2-yl]oxy}-[3,3′-bipyridine]-5-yl]acetic acid (12.4 mg, 23% yield, 515.67% purity). LCMS observed ion=516.3. ¹H NMR (500 MHz, METHANOL-d4) δ 8.15 (s, 1H), 7.91 (s, 1H), 7.56 (br d, J=10.7 Hz, 1H), 5.79 (s, 1H), 5.20 (quin, J=6.1 Hz, 1H), 3.14 (br s, 2H), 2.79 (br s, 2H), 2.66 (s, 3H), 2.06-1.98 (m, 1H), 1.45 (br s, 4H), 1.34 (d, J=6.1 Hz, 3H), 1.21 (s, 9H), 1.08-1.00 (m, 6H), 0.92 (s, 6H).

### Example 371

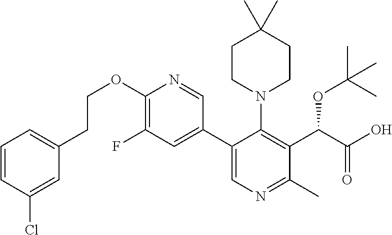 [SMILES: Cc1ncc(-c2cnc(OCCc3cccc(Cl)c3)c(F)c2)c(N2CCC(C)(C)CC2)c1[C@H](OC(C)(C)C)C(=O)O]

General Procedure H was followed. The experiment afforded the desired compound, (2S)-2-(tert-butoxy)-2-{6′-[2-(3-chlorophenyl)ethoxy]-4-(4,4-dimethylpiperidin-1-yl)-5′-fluoro-6-methyl-[3,3′-bipyridine]-5-yl}acetic acid (10.8 mg, 18% yield, 584.13% purity). LCMS observed ion=584.2. H NMR (500 MHz, METHANOL-d4) δ 8.09 (br s, 1H), 7.92 (br s, 1H), 7.56 (br d, J=10.4 Hz, 1H), 7.36 (br s, 1H), 7.33-7.23 (m, 3H), 5.75 (br s, 1H), 4.70-4.67 (m, 2H), 3.16 (br t, J=6.6 Hz, 2H), 3.12 (br s, 2H), 2.75 (br s, 2H), 2.65 (br s, 3H), 1.45 (br s, 4H), 1.20 (s, 9H), 0.91 (s, 6H).

### Example 372

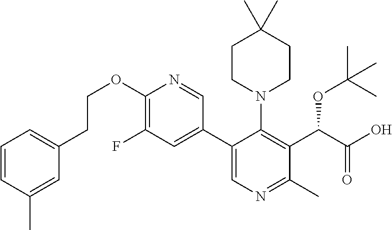 [SMILES: Cc1cccc(CCOc2ncc(-c3cnc(C)c([C@H](OC(C)(C)C)C(=O)O)c3N3CCC(C)(C)CC3)cc2F)c1]

General Procedure H was followed. The experiment afforded the desired compound, (2S)-2-(tert-butoxy)-2-[4-(4,4-dimethylpiperidin-1-yl)-5′-fluoro-6-methyl-6′-[2-(3-methylphenyl)ethoxy]-[3,3′-bipyridine]-5-yl]acetic acid (25.3 mg, 44% yield, 563.71% purity). LCMS observed ion=564.3. ¹H NMR (500 MHz, METHANOL-d4) δ 8.12 (s, 1H), 7.92 (s, 1H), 7.56 (br d, J=10.7 Hz, 1H), 7.19 (t, J=7.3 Hz, 1H), 7.15 (s, 1H), 7.11 (br d, J=7.6 Hz, 1H), 7.05 (br d, J=7.6 Hz, 1H), 5.78 (br s, 1H), 4.67-4.64 (m, 2H), 3.11 (br t, J=6.9 Hz, 2H), 3.11 (br s, 2H), 2.78 (br s, 2H), 2.65 (s, 3H), 2.34 (s, 3H), 1.45 (br s, 4H), 1.21 (s, 9H), 0.92 (s, 6H).

### Example 373

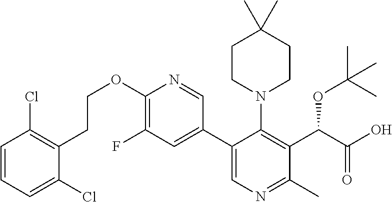 [SMILES: Cc1ncc(-c2cnc(OCCc3c(Cl)cccc3Cl)c(F)c2)c(N2CCC(C)(C)CC2)c1[C@H](OC(C)(C)C)C(=O)O]

General Procedure H was followed. The experiment afforded the desired compound, (2S)-2-(tert-butoxy)-2-{6′-[2-(2,6-dichlorophenyl)ethoxy]-4-(4,4-dimethylpiperidin-1-yl)-5′-fluoro-6-methyl-[3,3′-bipyridine]-5-yl}acetic acid (7.8 mg, 12% yield, 618.57% purity). LCMS observed ion=618.2. ¹H NMR (500 MHz, METHANOL-d4) δ 8.13 (br s, 1H), 7.91 (s, 1H), 7.56 (br d, J=10.7 Hz, 1H), 7.41 (d, J=7.4 Hz, 2H), 7.27-7.23 (m, 1H), 5.78 (br s, 1H), 4.77 (br t, J=6.4 Hz, 2H), 3.53 (br t, J=6.6 Hz, 2H), 2.78 (br s, 2H), 2.65 (s, 3H), 1.46 (br s, 4H), 1.21 (s, 9H), 0.94 (s, 6H).

### Example 374

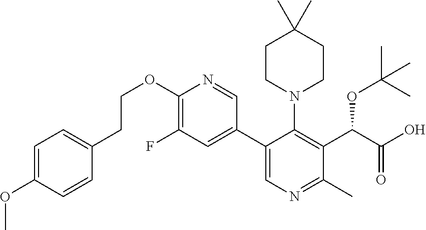 [SMILES: COc1ccc(CCOc2ncc(-c3cnc(C)c([C@H](OC(C)(C)C)C(=O)O)c3N3CCC(C)(C)CC3)cc2F)cc1]

General Procedure H was followed. The experiment afforded the desired compound, (2S)-2-(tert-butoxy)-2-[4-(4,4-dimethylpiperidin-1-yl)-5′-fluoro-6′-[2-(4-methoxyphenyl)ethoxy]-6-methyl-[3,3′-bipyridine]-5-yl]acetic acid (26.3 mg, 45% yield, 579.71% purity). LCMS observed ion=580.3. ¹H NMR (500 MHz, METHANOL-d4) δ 8.13 (s, 1H), 7.92 (s, 1H), 7.56 (br d, J=10.1 Hz, 1H), 7.26-7.22 (m, J=8.5 Hz, 2H), 6.89-6.86 (m, J=8.5 Hz, 2H), 5.79 (br s, 1H), 4.65-4.61 (m, 2H), 3.79 (s, 3H), 3.09 (br t, J=6.9 Hz, 2H), 2.78 (br s, 2H), 2.65 (s, 3H), 1.45 (br s, 4H), 1.21 (s, 9H), 0.92 (s, 6H).

### Example 375

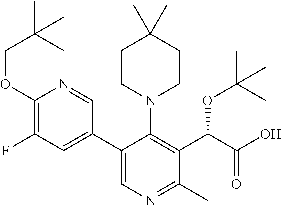 [SMILES: Cc1ncc(-c2cnc(OCC(C)(C)C)c(F)c2)c(N2CCC(C)(C)CC2)c1[C@H](OC(C)(C)C)C(=O)O]

General Procedure H was followed. The experiment afforded the desired compound, (2S)-2-(tert-butoxy)-2-[4-(4,4-dimethylpiperidin-1-yl)-6′-(2,2-dimethylpropoxy)-5′-fluoro-6-methyl-[3,3′-bipyridine]-5-yl]acetic acid (15.3 mg, 29% yield, 515.67% purity). LCMS observed ion=516.3. ¹H NMR (500 MHz, METHANOL-d4) δ 8.13 (s, 1H), 7.91 (s, 1H), 7.58 (br d, J=10.4 Hz, 1H), 5.78 (br s, 1H), 4.21-4.11 (m, 2H), 2.78 (br s, 2H), 2.65 (s, 3H), 1.46 (br s, 4H), 1.21 (s, 9H), 1.09 (s, 9H), 0.92 (s, 6H).

### Example 376

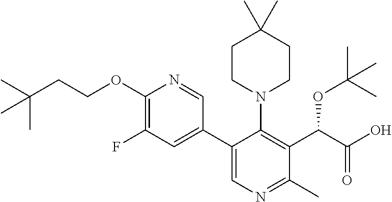 [SMILES: Cc1ncc(-c2cnc(OCCC(C)(C)C)c(F)c2)c(N2CCC(C)(C)CC2)c1[C@H](OC(C)(C)C)C(=O)O]

General Procedure H was followed. The experiment afforded the desired compound, (2S)-2-(tert-butoxy)-2-[6′-(3,3-dimethylbutoxy)-4-(4,4-dimethylpiperidin-1-yl)-5′-fluoro-6-methyl-[3,3′-bipyridine]-5-yl]acetic acid (16.3 mg, 30% yield, 529.7% purity). LCMS observed ion=530.3. ¹H NMR (500 MHz, METHANOL-d4) δ 8.15 (s, 1H), 7.93 (s, 1H), 7.57 (br d, J=10.7 Hz, 1H), 5.78 (br s, 1H), 4.62-4.50 (m, 2H), 2.79 (br s, 2H), 2.66 (s, 3H), 1.79 (t, J=7.2 Hz, 2H), 1.46 (br s, 4H), 1.21 (s, 9H), 1.06-1.02 (m, 9H), 0.93 (s, 6H).

### Example 377

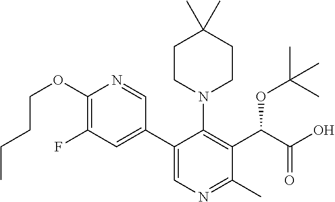 [SMILES: CCCCOc1ncc(-c2cnc(C)c([C@H](OC(C)(C)C)C(=O)O)c2N2CCC(C)(C)CC2)cc1F]

General Procedure H was followed. The experiment afforded the desired compound, (2S)-2-(tert-butoxy)-2-[6′-butoxy-4-(4,4-dimethylpiperidin-1-yl)-5′-fluoro-6-methyl-[3,3′-bipyridine]-5-yl]acetic acid (12.7 mg, 25% yield, 501.64% purity). LCMS observed ion=502.3. ¹H NMR (500 MHz, METHANOL-d4) δ 8.13 (s, 1H), 7.92 (s, 1H), 7.57 (br d, J=10.4 Hz, 1H), 5.78 (br s, 1H), 4.53-4.43 (m, 2H), 2.78 (br s, 2H), 2.65 (s, 3H), 1.84 (quin, J=7.1 Hz, 2H), 1.54 (sxt, J=7.3 Hz, 2H), 1.46 (br s, 4H), 1.21 (s, 9H), 1.05-1.00 (m, 3H), 0.92 (s, 6H).

### Example 378

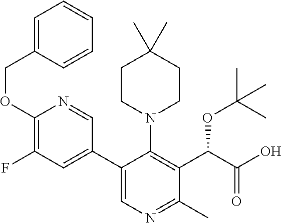 [SMILES: Cc1ncc(-c2cnc(OCc3ccccc3)c(F)c2)c(N2CCC(C)(C)CC2)c1[C@H](OC(C)(C)C)C(=O)O]

General Procedure H was followed. The experiment afforded the desired compound, (2S)-2-[6′-(benzyloxy)-4-(4,4-dimethylpiperidin-1-yl)-5′-fluoro-6-methyl-[3,3′-bipyridine]-5-yl]-2-(tert-butoxy)acetic acid (11.9 mg, 22% yield, 535.66% purity). LCMS observed ion=536.3. ¹H NMR (500 MHz, METHANOL-d4) δ 8.14 (s, 1H), 7.94 (s, 1H), 7.60 (br d, J=10.1 Hz, 1H), 7.50 (br d, J=7.0 Hz, 2H), 7.41-7.32 (m, 3H), 5.78 (s, 1H), 5.60 (d, J=12.2 Hz, 1H), 5.52 (d, J=12.5 Hz, 1H), 2.78 (br s, 2H), 2.65 (s, 3H), 1.45 (br s, 4H), 1.21 (s, 9H), 0.90 (s, 6H).

### Example 379

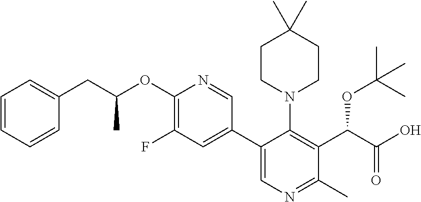 [SMILES: Cc1ncc(-c2cnc(O[C@@H](C)Cc3ccccc3)c(F)c2)c(N2CCC(C)(C)CC2)c1[C@H](OC(C)(C)C)C(=O)O]

General Procedure H was followed. The experiment afforded the desired compound, (2S)-2-(tert-butoxy)-2-[4-(4,4-dimethylpiperidin-1-yl)-5′-fluoro-6-methyl-6′-{[(2S)-1-phenylpropan-2-yl]oxy}-[3,3′-bipyridine]-5-yl]acetic acid (13.6 mg, 24% yield, 563.71% purity). LCMS observed ion=564.3. ¹H NMR (500 MHz, METHANOL-d4) δ 8.11 (s, 1H), 7.88 (s, 1H), 7.52 (br d, J=10.1 Hz, 1H), 7.32-7.25 (m, 4H), 7.21-7.17 (m, 1H), 5.77 (s, 1H), 5.62-5.57 (m, 1H), 3.14 (br dd, J=13.7, 7.0 Hz, 1H), 2.97 (br dd, J=13.7, 5.8 Hz, 1H), 2.76 (br s, 2H), 2.65 (s, 3H), 1.45 (br s, 4H), 1.40 (br d, J=6.1 Hz, 3H), 1.20 (s, 9H), 0.92 (s, 6H).

### Example 380

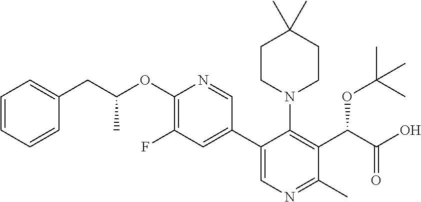 [SMILES: Cc1ncc(-c2cnc(O[C@H](C)Cc3ccccc3)c(F)c2)c(N2CCC(C)(C)CC2)c1[C@H](OC(C)(C)C)C(=O)O]

General Procedure H was followed. The experiment afforded the desired compound, (2S)-2-(tert-butoxy)-2-[4-(4,4-dimethylpiperidin-1-yl)-5′-fluoro-6-methyl-6′-{[(2R)-1-phenylpropan-2-yl]oxy}-[3,3′-bipyridine]-5-yl]acetic acid (12.2 mg, 21% yield, 563.71% purity). LCMS observed ion=564.3.

### Example 381

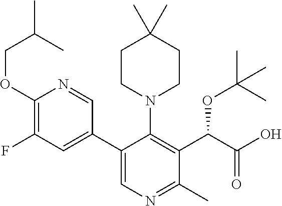 [SMILES: Cc1ncc(-c2cnc(OCC(C)C)c(F)c2)c(N2CCC(C)(C)CC2)c1[C@H](OC(C)(C)C)C(=O)O]

General Procedure H was followed. The experiment afforded the desired compound, (2S)-2-(tert-butoxy)-2-[4-(4,4-dimethylpiperidin-1-yl)-5′-fluoro-6-methyl-6′-(2-methylpropoxy)-[3,3′-bipyridine]-5-yl]acetic acid (12.8 mg, 25% yield, 501.64% purity). LCMS observed ion=502.3.

### Example 382

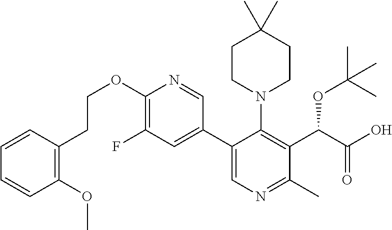 [SMILES: COc1ccccc1CCOc1ncc(-c2cnc(C)c([C@H](OC(C)(C)C)C(=O)O)c2N2CCC(C)(C)CC2)cc1F]

General Procedure H was followed. The experiment afforded the desired compound, (2S)-2-(tert-butoxy)-2-[4-(4,4-dimethylpiperidin-1-yl)-5′-fluoro-6′-[2-(2-methoxyphenyl)ethoxy]-6-methyl-[3,3′-bipyridine]-5-yl]acetic acid (16.5 mg, 28% yield, 579.71% purity). LCMS observed ion=580.3. ¹H NMR (500 MHz, METHANOL-d4) δ 8.13 (br s, 1H), 7.90 (s, 1H), 7.55 (br d, J=10.4 Hz, 1H), 7.25-7.19 (m, 2H), 6.97 (d, J=7.9 Hz, 1H), 6.89 (t, J=7.5 Hz, 1H), 5.76 (br s, 1H), 4.66-4.62 (m, 2H), 3.86 (s, 3H), 3.15 (br t, J=6.7 Hz, 2H), 2.79 (br s, 2H), 2.66 (s, 3H), 1.46 (br s, 4H), 1.21 (s, 9H), 0.93 (s, 6H).

### Example 383

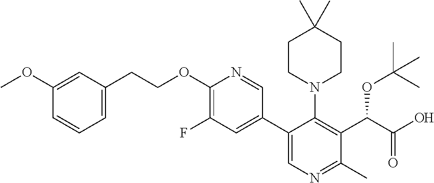 [SMILES: COc1cccc(CCOc2ncc(-c3cnc(C)c([C@H](OC(C)(C)C)C(=O)O)c3N3CCC(C)(C)CC3)cc2F)c1]

General Procedure H was followed. The experiment afforded the desired compound, (2S)-2-(tert-butoxy)-2-[4-(4,4-dimethylpiperidin-1-yl)-5′-fluoro-6′-[2-(3-methoxyphenyl)ethoxy]-6-methyl-[3,3′-bipyridine]-5-yl]acetic acid (17.5 mg, 30% yield, 579.71% purity). LCMS observed ion=580.3. ¹H NMR (500 MHz, METHANOL-d4) δ 8.14 (s, 1H), 7.92 (s, 1H), 7.57 (br d, J=11.3 Hz, 1H), 7.22 (t, J=7.9 Hz, 1H), 6.91 (br s, 2H), 6.80 (br d, J=8.2 Hz, 1H), 5.76 (br s, 1H), 4.67 (br t, J=6.1 Hz, 2H), 3.80 (s, 3H), 3.13 (br t, J=6.9 Hz, 2H), 2.79 (br s, 2H), 2.66 (s, 3H), 1.45 (br s, 4H), 1.21 (s, 9H), 0.92 (s, 6H).

### Example 384

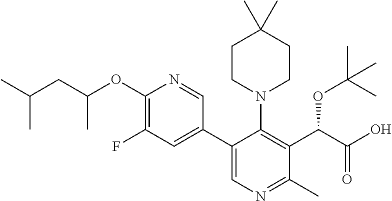 [SMILES: Cc1ncc(-c2cnc(OC(C)CC(C)C)c(F)c2)c(N2CCC(C)(C)CC2)c1[C@H](OC(C)(C)C)C(=O)O]

General Procedure H was followed. The experiment afforded the desired compound, (2S)-2-(tert-butoxy)-2-[4-(4,4-dimethylpiperidin-1-yl)-5′-fluoro-6-methyl-6′-[(4-methylpentan-2-yl)oxy]-[3,3′-bipyridine]-5-yl]acetic acid (11.6 mg, 21% yield, 529.7% purity). LCMS observed ion=530.3. ¹H NMR (500 MHz, METHANOL-d4) δ 8.17 (br s, 1H), 7.92 (br s, 1H), 7.57 (br d, J=9.5 Hz, 1H), 5.77 (br s, 1H), 5.55-5.46 (m, 1H), 3.13 (br s, 2H), 2.81 (br s, 2H), 2.67 (s, 3H), 1.84-1.76 (m, 2H), 1.52-1.42 (m, 5H), 1.41-1.36 (m, J=6.4, 6.4 Hz, 3H), 1.21 (s, 9H), 0.98 (br d, J=6.1 Hz, 3H), 0.94 (br d, J=5.8 Hz, 3H), 0.92 (s, 6H).

### Example 385

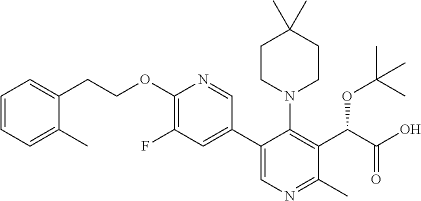 [SMILES: Cc1ccccc1CCOc1ncc(-c2cnc(C)c([C@H](OC(C)(C)C)C(=O)O)c2N2CCC(C)(C)CC2)cc1F]

General Procedure H was followed. The experiment afforded the desired compound, (2S)-2-(tert-butoxy)-2-[4-(4,4-dimethylpiperidin-1-yl)-5′-fluoro-6-methyl-6′-[2-(2-methylphenyl)ethoxy]-[3,3′-bipyridine]-5-yl]acetic acid (18.2 mg, 32% yield, 563.71% purity). LCMS observed ion=564.3. ¹H NMR (500 MHz, METHANOL-d4) δ 8.14 (s, 1H), 7.92 (s, 1H), 7.57 (br d, J=10.1 Hz, 1H), 7.24 (br d, J=4.9 Hz, 1H), 7.17 (br d, J=3.7 Hz, 1H), 7.15-7.11 (m, 2H), 5.76 (s, 1H), 4.69-4.63 (m, 2H), 3.18 (br t, J=7.0 Hz, 2H), 2.79 (br s, 2H), 2.66 (s, 3H), 2.42 (s, 3H), 1.46 (br s, 4H), 1.21 (s, 9H), 0.93 (s, 6H).

### Example 386

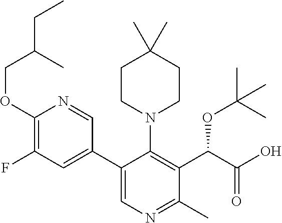 [SMILES: CCC(C)COc1ncc(-c2cnc(C)c([C@H](OC(C)(C)C)C(=O)O)c2N2CCC(C)(C)CC2)cc1F]

General Procedure H was followed. The experiment afforded the desired compound, (2S)-2-(tert-butoxy)-2-[4-(4,4-dimethylpiperidin-1-yl)-5′-fluoro-6-methyl-6′-(2-methylbutoxy)-[3,3′-bipyridine]-5-yl]acetic acid (16.9 mg, 32% yield, 515.67% purity). LCMS observed ion=516.3. ¹H NMR (500 MHz, METHANOL-d4) δ 8.16 (br s, 1H), 7.92 (s, 1H), 7.58 (br d, J=10.7 Hz, 1H), 5.74 (br s, 1H), 4.40-4.24 (m, 2H), 3.14 (br s, 2H), 2.81 (br s, 2H), 2.67 (br s, 3H), 1.98-1.91 (m, 1H), 1.61 (dt, J=13.8, 7.0 Hz, 1H), 1.46 (br s, 4H), 1.40-1.31 (m, 1H), 1.21 (s, 9H), 1.07 (d, J=6.7 Hz, 3H), 1.00 (t, J=7.5 Hz, 3H), 0.93 (s, 6H).

### Example 387

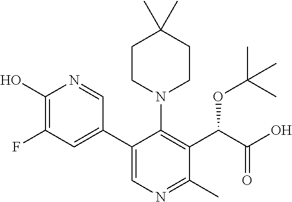 [SMILES: Cc1ncc(-c2cnc(O)c(F)c2)c(N2CCC(C)(C)CC2)c1[C@H](OC(C)(C)C)C(=O)O]

The desired compound was isolated as a second product during the synthesis of Example EG-195. The isolated material is (2S)-2-(tert-butoxy)-2-[4-(4,4-dimethylpiperidin-1-yl)-5′-fluoro-6′-hydroxy-6-methyl-[3,3′-bipyridine]-5-yl]acetic acid (14.3 mg, 32% yield, 445.54% purity). LCMS observed ion=446.2. ¹H NMR (500 MHz, METHANOL-d4) δ 8.00 (s, 1H), 7.48 (br d, J=10.1 Hz, 1H), 7.29 (s, 1H), 5.64 (s, 1H), 2.61 (s, 3H), 1.17 (s, 9H), 0.94 (br s, 6H); the piperidine protons were not observed due to line broadening.

### Example 388

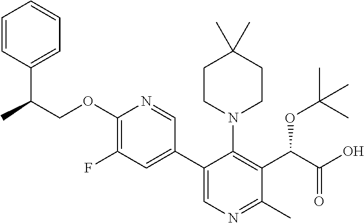 [SMILES: Cc1ncc(-c2cnc(OC[C@@H](C)c3ccccc3)c(F)c2)c(N2CCC(C)(C)CC2)c1[C@H](OC(C)(C)C)C(=O)O]

General Procedure H was followed. The experiment afforded the desired compound, (2S)-2-(tert-butoxy)-2-[4-(4,4-dimethylpiperidin-1-yl)-5′-fluoro-6-methyl-6′-[(2S)-2-phenylpropoxy]-[3,3′-bipyridine]-5-yl]acetic acid (19 mg, 33% yield, 563.71% purity). LCMS observed ion=564.3. ¹H NMR (500 MHz, METHANOL-d4) δ 8.14 (s, 1H), 7.91 (s, 1H), 7.56 (br d, J=11.3 Hz, 1H), 7.35-7.31 (m, 4H), 7.25-7.21 (m, 1H), 5.78 (br s, 1H), 4.62 (dd, J=10.4, 6.4 Hz, 1H), 4.50 (dd, J=10.4, 7.3 Hz, 1H), 3.37-3.34 (m, 1H), 2.79 (br s, 2H), 2.66 (s, 3H), 1.46 (br s, 4H), 1.43 (d, J=7.0 Hz, 3H), 1.21 (s, 9H), 0.93 (s, 6H).

### Example 389

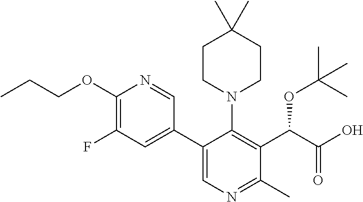 [SMILES: CCCOc1ncc(-c2cnc(C)c([C@H](OC(C)(C)C)C(=O)O)c2N2CCC(C)(C)CC2)cc1F]

General Procedure H was followed. The experiment afforded the desired compound, (2S)-2-(tert-butoxy)-2-[4-(4,4-dimethylpiperidin-1-yl)-5′-fluoro-6-methyl-6′-propoxy-[3,3′-bipyridine]-5-yl]acetic acid (15.7 mg, 32% yield, 487.62% purity). LCMS observed ion=488.3. ¹H NMR (500 MHz, METHANOL-d4) δ 8.15 (s, 1H), 7.92 (s, 1H), 7.58 (br d, J=10.7 Hz, 1H), 5.79 (s, 1H), 4.47-4.39 (m, 2H), 2.79 (br s, 2H), 2.66 (s, 3H), 1.92-1.85 (m, 2H), 1.46 (br s, 4H), 1.21 (s, 9H), 1.08 (t, J=7.5 Hz, 3H), 0.93 (s, 6H).

### Example 390

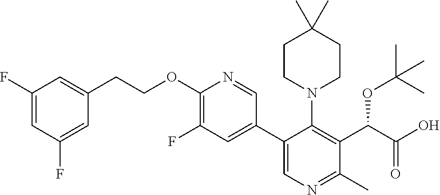 [SMILES: Cc1ncc(-c2cnc(OCCc3cc(F)cc(F)c3)c(F)c2)c(N2CCC(C)(C)CC2)c1[C@H](OC(C)(C)C)C(=O)O]

General Procedure H was followed. The experiment afforded the desired compound, (2S)-2-(tert-butoxy)-2-{6′-[2-(3,5-difluorophenyl)ethoxy]-4-(4,4-dimethylpiperidin-1-yl)-5′-fluoro-6-methyl-[3,3′-bipyridine]-5-yl}acetic acid (6.8 mg, 11% yield, 585.67% purity). LCMS observed ion=586.2. ¹H NMR (500 MHz, METHANOL-d4) δ 8.15 (s, 1H), 7.94 (s, 1H), 7.59 (br d, J=10.4 Hz, 1H), 6.96 (br d, J=7.0 Hz, 2H), 6.82 (br t, J=8.5 Hz, 1H), 5.78 (s, 1H), 4.71 (t, J=6.4 Hz, 2H), 3.19 (t, J=6.4 Hz, 2H), 3.14 (br s, 2H), 2.79 (br s, 2H), 2.66 (s, 3H), 1.46 (br s, 4H), 1.21 (s, 9H), 0.92 (s, 6H).

### Example 391

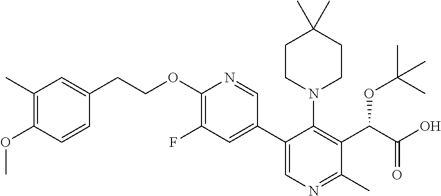 [SMILES: COc1ccc(CCOc2ncc(-c3cnc(C)c([C@H](OC(C)(C)C)C(=O)O)c3N3CCC(C)(C)CC3)cc2F)cc1C]

General Procedure H was followed. The experiment afforded the desired compound, (2S)-2-(tert-butoxy)-2-[4-(4,4-dimethylpiperidin-1-yl)-5′-fluoro-6′-[2-(4-methoxy-3-methylphenyl)ethoxy]-6-methyl-[3,3′-bipyridine]-5-yl]acetic acid (15.5 mg, 26% yield, 593.74% purity). LCMS observed ion=594.3. ¹H NMR (500 MHz, METHANOL-d4) δ 8.10 (s, 1H), 7.91 (s, 1H), 7.55 (br d, J=9.8 Hz, 1H), 7.11-7.08 (m, 2H), 6.84 (d, J=7.9 Hz, 1H), 5.77 (s, 1H), 4.64-4.59 (m, 2H), 3.82 (s, 3H), 3.05 (t, J=7.0 Hz, 2H), 2.77 (br s, 2H), 2.65 (s, 3H), 2.18 (s, 3H), 1.45 (br s, 4H), 1.20 (s, 9H), 0.91 (s, 6H).

### Example 392

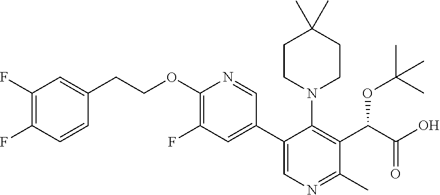 [SMILES: Cc1ncc(-c2cnc(OCCc3ccc(F)c(F)c3)c(F)c2)c(N2CCC(C)(C)CC2)c1[C@H](OC(C)(C)C)C(=O)O]

General Procedure H was followed. The experiment afforded the desired compound, (2S)-2-(tert-butoxy)-2-{6′-[2-(3,4-difluorophenyl)ethoxy]-4-(4,4-dimethylpiperidin-1-yl)-5′-fluoro-6-methyl-[3,3′-bipyridine]-5-yl}acetic acid (8.1 mg, 13% yield, 585.67% purity). LCMS observed ion=586.2. ¹H NMR (500 MHz, METHANOL-d4) δ 8.13 (s, 1H), 7.92 (s, 1H), 7.57 (br d, J=10.4 Hz, 1H), 7.28-7.22 (m, 1H), 7.22-7.17 (m, 1H), 7.14 (br s, 1H), 5.78 (s, 1H), 4.70-4.66 (m, 2H), 3.15 (br t, J=6.6 Hz, 2H), 2.78 (br s, 2H), 2.65 (s, 3H), 1.45 (br s, 4H), 1.21 (s, 9H), 0.92 (s, 6H).

### Example 393

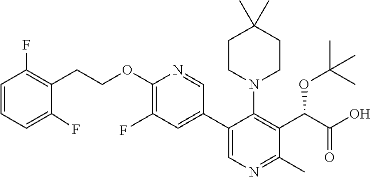 [SMILES: Cc1ncc(-c2cnc(OCCc3c(F)cccc3F)c(F)c2)c(N2CCC(C)(C)CC2)c1[C@H](OC(C)(C)C)C(=O)O]

General Procedure H was followed. The experiment afforded the desired compound, (2S)-2-(tert-butoxy)-2-{6′-[2-(2,6-difluorophenyl)ethoxy]-4-(4,4-dimethylpiperidin-1-yl)-5′-fluoro-6-methyl-[3,3′-bipyridine]-5-yl}acetic acid (11.6 mg, 19% yield, 585.67% purity). LCMS observed ion=586.2. ¹H NMR (500 MHz, METHANOL-d4) δ 8.13 (s, 1H), 7.91 (s, 1H), 7.56 (br d, J=10.4 Hz, 1H), 7.33-7.27 (m, 1H), 6.96 (t, J=7.8 Hz, 2H), 5.78 (s, 1H), 4.71 (br t, J=5.8 Hz, 2H), 3.24 (br t, J=6.3 Hz, 2H), 2.78 (br s, 2H), 2.66 (s, 3H), 1.46 (br s, 4H), 1.21 (s, 9H), 0.93 (s, 6H).

### Example 394

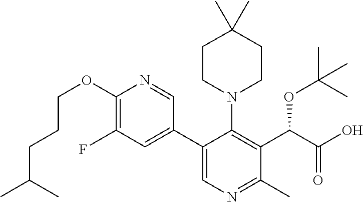 [SMILES: Cc1ncc(-c2cnc(OCCCC(C)C)c(F)c2)c(N2CCC(C)(C)CC2)c1[C@H](OC(C)(C)C)C(=O)O]

General Procedure H was followed. The experiment afforded the desired compound, (2S)-2-(tert-butoxy)-2-[4-(4,4-dimethylpiperidin-1-yl)-5′-fluoro-6-methyl-6′-[(4-methylpentyl)oxy]-[3,3′-bipyridine]-5-yl]acetic acid (12 mg, 22% yield, 529.7% purity). LCMS observed ion=530.3. ¹H NMR (500 MHz, METHANOL-d4) δ 8.15 (s, 1H), 7.92 (s, 1H), 7.57 (br d, J=10.7 Hz, 1H), 5.78 (s, 1H), 4.51-4.42 (m, 2H), 2.80 (br s, 2H), 2.66 (s, 3H), 1.90-1.83 (m, 2H), 1.65 (dt, J=13.2, 6.7 Hz, 1H), 1.46 (br s, 4H), 1.42-1.36 (m, 2H), 1.21 (s, 9H), 0.96 (d, J=6.4 Hz, 6H), 0.93 (s, 6H).

### Example 395

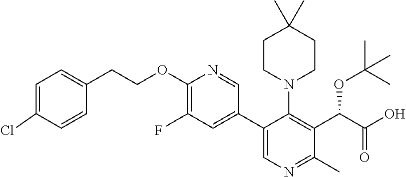 [SMILES: Cc1ncc(-c2cnc(OCCc3ccc(Cl)cc3)c(F)c2)c(N2CCC(C)(C)CC2)c1[C@H](OC(C)(C)C)C(=O)O]

General Procedure H was followed. The experiment afforded the desired compound, (2S)-2-(tert-butoxy)-2-{6′-[2-(4-chlorophenyl)ethoxy]-4-(4,4-dimethylpiperidin-1-yl)-5′-fluoro-6-methyl-[3,3′-bipyridine]-5-yl}acetic acid (17.4 mg, 29% yield, 584.13% purity). LCMS observed ion=584.2. H NMR (500 MHz, METHANOL-d4) δ 8.14 (s, 1H), 7.92 (s, 1H), 7.57 (br d, J=10.1 Hz, 1H), 7.34-7.30 (m, 4H), 5.77 (s, 1H), 4.70-4.66 (m, 2H), 3.15 (t, J=6.7 Hz, 2H), 2.79 (br s, 2H), 2.66 (s, 3H), 1.45 (br s, 4H), 1.21 (s, 9H), 0.92 (s, 6H).

### Example 396

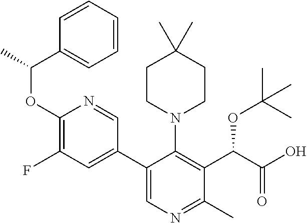 [SMILES: Cc1ncc(-c2cnc(O[C@H](C)c3ccccc3)c(F)c2)c(N2CCC(C)(C)CC2)c1[C@H](OC(C)(C)C)C(=O)O]

General Procedure H was followed. The experiment afforded the desired compound, (2S)-2-(tert-butoxy)-2-[4-(4,4-dimethylpiperidin-1-yl)-5′-fluoro-6-methyl-6′-[(1R)-1-phenylethoxy]-[3,3′-bipyridine]-5-yl]acetic acid (18.3 mg, 33% yield, 549.69% purity). LCMS observed ion=550.3. ¹H NMR (500 MHz, METHANOL-d4) δ 8.11 (br s, 1H), 7.83 (s, 1H), 7.57 (br d, J=10.1 Hz, 1H), 7.45 (d, J=7.6 Hz, 2H), 7.34 (t, J=7.6 Hz, 2H), 7.29-7.23 (m, 1H), 6.34 (q, J=6.2 Hz, 1H), 5.72 (br s, 1H), 2.69 (br s, 2H), 2.64 (br s, 3H), 1.71 (d, J=6.4 Hz, 3H), 1.39 (br s, 4H), 1.19 (s, 9H), 0.82 (br s, 6H).

### Example 397

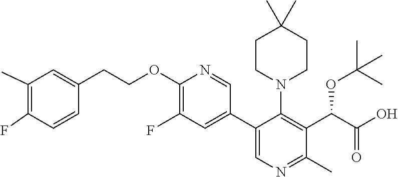 [SMILES: Cc1cc(CCOc2ncc(-c3cnc(C)c([C@H](OC(C)(C)C)C(=O)O)c3N3CCC(C)(C)CC3)cc2F)ccc1F]

General Procedure H was followed. The experiment afforded the desired compound, (2S)-2-(tert-butoxy)-2-[4-(4,4-dimethylpiperidin-1-yl)-5′-fluoro-6′-[2-(4-fluoro-3-methylphenyl)ethoxy]-6-methyl-[3,3′-bipyridine]-5-yl]acetic acid (20.1 mg, 34% yield, 581.71% purity). LCMS observed ion=582.2. ¹H NMR (500 MHz, METHANOL-d4) δ 8.13 (br s, 1H), 7.92 (s, 1H), 7.57 (br d, J=10.4 Hz, 1H), 7.20 (br d, J=7.3 Hz, 1H), 7.13 (br t, J=6.8 Hz, 1H), 6.96 (t, J=9.0 Hz, 1H), 5.78 (br s, 1H), 4.67-4.62 (m, 2H), 3.10 (t, J=6.7 Hz, 2H), 2.78 (br s, 2H), 2.65 (s, 3H), 2.26 (s, 3H), 1.45 (br s, 4H), 1.21 (s, 9H), 0.92 (s, 6H).

### Example 398

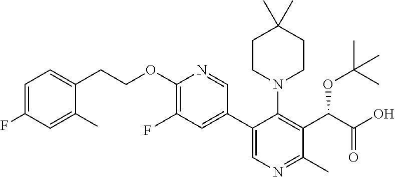 [SMILES: Cc1cc(F)ccc1CCOc1ncc(-c2cnc(C)c([C@H](OC(C)(C)C)C(=O)O)c2N2CCC(C)(C)CC2)cc1F]

General Procedure H was followed. The experiment afforded the desired compound, (2S)-2-(tert-butoxy)-2-[4-(4,4-dimethylpiperidin-1-yl)-5′-fluoro-6′-[2-(4-fluoro-2-methylphenyl)ethoxy]-6-methyl-[3,3′-bipyridine]-5-yl]acetic acid (21.3 mg, 36% yield, 581.71% purity). LCMS observed ion=582.3. ¹H NMR (500 MHz, METHANOL-d4) δ 8.13 (br s, 1H), 7.91 (s, 1H), 7.56 (br d, J=10.7 Hz, 1H), 7.26 (t, J=7.0 Hz, 1H), 6.93 (br d, J=10.1 Hz, 1H), 6.86 (t, J=8.2 Hz, 1H), 5.78 (br s, 1H), 4.65 (t, J=6.7 Hz, 2H), 3.16 (br t, J=7.0 Hz, 2H), 2.78 (br s, 2H), 2.65 (s, 3H), 2.43 (s, 3H), 1.46 (br s, 4H), 1.21 (s, 9H), 0.92 (s, 6H).

### Example 399

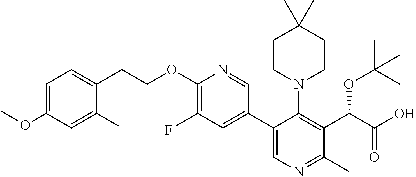 [SMILES: COc1ccc(CCOc2ncc(-c3cnc(C)c([C@H](OC(C)(C)C)C(=O)O)c3N3CCC(C)(C)CC3)cc2F)c(C)c1]

General Procedure H was followed. The experiment afforded the desired compound, (2S)-2-(tert-butoxy)-2-[4-(4,4-dimethylpiperidin-1-yl)-5′-fluoro-6′-[2-(4-methoxy-2-methylphenyl)ethoxy]-6-methyl-[3,3′-bipyridine]-5-yl]acetic acid (18.4 mg, 30% yield, 593.74% purity). LCMS observed ion=594.3. ¹H NMR (500 MHz, METHANOL-d4) δ 8.13 (s, 1H), 7.91 (s, 1H), 7.56 (br d, J=10.1 Hz, 1H), 7.16 (d, J=8.2 Hz, 1H), 6.75 (s, 1H), 6.71 (d, J=8.0 Hz, 1H), 5.78 (br s, 1H), 4.64-4.59 (m, 2H), 3.77 (s, 3H), 3.11 (br t, J=7.0 Hz, 2H), 2.78 (br s, 2H), 2.65 (s, 3H), 2.39 (s, 3H), 1.45 (br s, 4H), 1.21 (s, 9H), 0.92 (s, 6H).

### Example 400

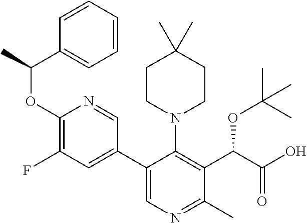 [SMILES: Cc1ncc(-c2cnc(O[C@@H](C)c3ccccc3)c(F)c2)c(N2CCC(C)(C)CC2)c1[C@H](OC(C)(C)C)C(=O)O]

General Procedure H was followed. The experiment afforded the desired compound, (2S)-2-(tert-butoxy)-2-[4-(4,4-dimethylpiperidin-1-yl)-5′-fluoro-6-methyl-6′-[(1S)-1-phenylethoxy]-[3,3′-bipyridine]-5-yl]acetic acid (20 mg, 36% yield, 549.69% purity). LCMS observed ion=550.3. ¹H NMR (500 MHz, METHANOL-d4) δ 8.11 (s, 1H), 7.86 (s, 1H), 7.56 (br d, J=10.1 Hz, 1H), 7.47 (d, J=7.3 Hz, 2H), 7.35 (t, J=7.5 Hz, 2H), 7.29-7.25 (m, 1H), 6.35 (q, J=6.6 Hz, 1H), 5.76 (br s, 1H), 2.74 (br s, 2H), 2.64 (s, 3H), 1.71 (d, J=6.4 Hz, 3H), 1.42 (br s, 4H), 1.20 (s, 9H), 0.91 (s, 6H).

### Example 401

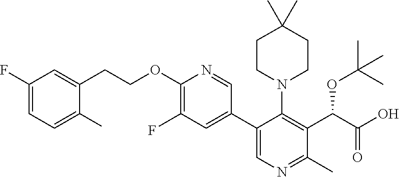 [SMILES: Cc1ccc(F)cc1CCOc1ncc(-c2cnc(C)c([C@H](OC(C)(C)C)C(=O)O)c2N2CCC(C)(C)CC2)cc1F]

General Procedure H was followed. The experiment afforded the desired compound, (2S)-2-(tert-butoxy)-2-[4-(4,4-dimethylpiperidin-1-yl)-5′-fluoro-6′-[2-(5-fluoro-2-methylphenyl)ethoxy]-6-methyl-[3,3′-bipyridine]-5-yl]acetic acid (17.9 mg, 30% yield, 581.71% purity). LCMS observed ion=582.3. H NMR (500 MHz, DMSO-d6) δ 8.08 (s, 1H), 7.93 (s, 1H), 7.75 (br d, J=10.7 Hz, 1H), 7.20 (br t, J=7.2 Hz, 1H), 7.07 (brd, J=10.1 Hz, 1H), 6.95 (brt, J=8.2 Hz, 1H), 5.78 (br s, 1H), 4.67-4.58 (m, 2H), 3.09 (brt, J=6.7 Hz, 2H), 2.49-2.47 (m, 3H), 2.29 (s, 3H), 2.22 (br s, 2H), 1.53 (br s, 1H), 1.31 (br s, 3H), 1.11 (s, 9H), 0.89 (br s, 3H), 0.74 (br s, 3H).

### Example 402

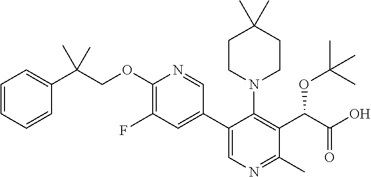 [SMILES: Cc1ncc(-c2cnc(OCC(C)(C)c3ccccc3)c(F)c2)c(N2CCC(C)(C)CC2)c1[C@H](OC(C)(C)C)C(=O)O]

General Procedure H was followed. The experiment afforded the desired compound, (2S)-2-(tert-butoxy)-2-[4-(4,4-dimethylpiperidin-1-yl)-5′-fluoro-6-methyl-6′-(2-methyl-2-phenylpropoxy)-[3,3′-bipyridine]-5-yl]acetic acid (14.3 mg, 24% yield, 577.74% purity). LCMS observed ion=578.3. ¹H NMR (500 MHz, METHANOL-d4) δ 8.11 (s, 1H), 7.89 (s, 1H), 7.56-7.48 (m, 3H), 7.33 (t, J=7.6 Hz, 2H), 7.26-7.19 (m, 1H), 5.77 (br s, 1H), 4.52 (s, 2H), 2.75 (br s, 2H), 2.65 (s, 3H), 1.51 (s, 6H), 1.45 (br s, 4H), 1.21 (s, 9H), 0.92 (s, 6H).

### Example 403

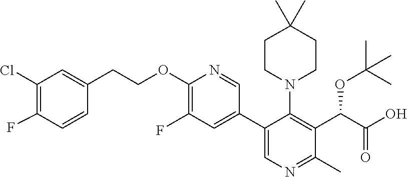 [SMILES: Cc1ncc(-c2cnc(OCCc3ccc(F)c(Cl)c3)c(F)c2)c(N2CCC(C)(C)CC2)c1[C@H](OC(C)(C)C)C(=O)O]

General Procedure H was followed. The experiment afforded the desired compound, (2S)-2-(tert-butoxy)-2-{6′-[2-(3-chloro-4-fluorophenyl)ethoxy]-4-(4,4-dimethylpiperidin-1-yl)-5′-fluoro-6-methyl-[3,3′-bipyridine]-5-yl}acetic acid (13.4 mg, 22% yield, 602.12% purity). LCMS observed ion=602.2. ¹H NMR (500 MHz, METHANOL-d4) δ 8.13 (s, 1H), 7.92 (s, 1H), 7.57 (br d, J=10.7 Hz, 1H), 7.46 (br d, J=7.0 Hz, 1H), 7.32-7.28 (m, 1H), 7.18 (t, J=8.5 Hz, 1H), 5.78 (br s, 1H), 4.70-4.66 (m, 2H), 3.15 (br t, J=6.6 Hz, 2H), 2.78 (br s, 2H), 2.65 (s, 3H), 1.45 (br s, 4H), 1.21 (s, 9H), 0.92 (s, 6H).

### Example 404

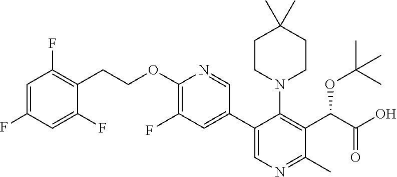 [SMILES: Cc1ncc(-c2cnc(OCCc3c(F)cc(F)cc3F)c(F)c2)c(N2CCC(C)(C)CC2)c1[C@H](OC(C)(C)C)C(=O)O]

General Procedure H was followed. The experiment afforded the desired compound, (2S)-2-(tert-butoxy)-2-[4-(4,4-dimethylpiperidin-1-yl)-5′-fluoro-6-methyl-6′-[2-(2,4,6-trifluorophenyl)ethoxy]-[3,3′-bipyridine]-5-yl]acetic acid (14.4 mg, 23% yield, 603.66% purity). LCMS observed ion=604.2. ¹H NMR (500 MHz, METHANOL-d4) δ 8.12 (s, 1H), 7.91 (s, 1H), 7.56 (br d, J=9.8 Hz, 1H), 6.86 (br t, J=8.4 Hz, 2H), 5.78 (br s, 1H), 4.69 (t, J=6.1 Hz, 2H), 3.21 (brt, J=6.3 Hz, 2H), 2.77 (br s, 2H), 2.65 (s, 3H), 1.46 (br s, 4H), 1.21 (s, 9H), 0.92 (s, 6H).

### Example 405

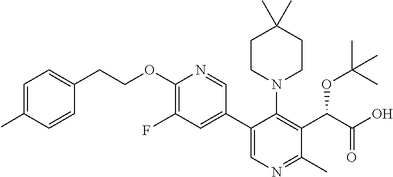 [SMILES: Cc1ccc(CCOc2ncc(-c3cnc(C)c([C@H](OC(C)(C)C)C(=O)O)c3N3CCC(C)(C)CC3)cc2F)cc1]

General Procedure H was followed. The experiment afforded the desired compound, (2S)-2-(tert-butoxy)-2-[4-(4,4-dimethylpiperidin-1-yl)-5′-fluoro-6-methyl-6′-[2-(4-methylphenyl)ethoxy]-[3,3′-bipyridine]-5-yl]acetic acid (17.7 mg, 31% yield, 563.71% purity). LCMS observed ion=564.3. ¹H NMR (500 MHz, METHANOL-d4) δ 8.13 (s, 1H), 7.92 (s, 1H), 7.56 (br d, J=10.1 Hz, 1H), 7.22-7.18 (m, J=7.6 Hz, 2H), 7.15-7.11 (m, J=7.6 Hz, 2H), 5.78 (s, 1H), 4.66-4.62 (m, 2H), 3.10 (t, J=6.9 Hz, 2H), 2.78 (br s, 2H), 2.66 (s, 3H), 2.32 (s, 3H), 1.46 (br s, 4H), 1.21 (s, 9H), 0.92 (s, 6H).

### Example 406

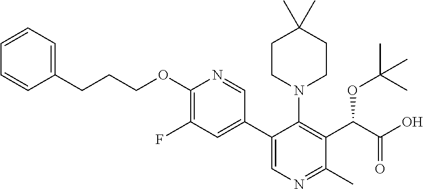 [SMILES: Cc1ncc(-c2cnc(OCCCc3ccccc3)c(F)c2)c(N2CCC(C)(C)CC2)c1[C@H](OC(C)(C)C)C(=O)O]

General Procedure H was followed. The experiment afforded the desired compound, (2S)-2-(tert-butoxy)-2-[4-(4,4-dimethylpiperidin-1-yl)-5′-fluoro-6-methyl-6′-(3-phenylpropoxy)-[3,3′-bipyridine]-5-yl]acetic acid (15.6 mg, 27% yield, 563.71% purity). LCMS observed ion=564.3. ¹H NMR (500 MHz, METHANOL-d4) δ 8.14 (s, 1H), 7.91 (s, 1H), 7.59 (br d, J=11.0 Hz, 1H), 7.30-7.23 (m, 4H), 7.21-7.17 (m, 1H), 5.78 (br s, 1H), 4.50-4.43 (m, 2H), 2.83 (t, J=7.6 Hz, 2H), 2.83 (br s, 2H), 2.66 (s, 3H), 2.17 (quin, J=6.9 Hz, 2H), 1.46 (br s, 4H), 1.21 (s, 9H), 0.92 (s, 6H).

### Example 407

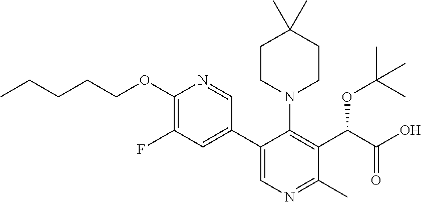 [SMILES: CCCCCOc1ncc(-c2cnc(C)c([C@H](OC(C)(C)C)C(=O)O)c2N2CCC(C)(C)CC2)cc1F]

General Procedure H was followed. The experiment afforded the desired compound, (2S)-2-(tert-butoxy)-2-[4-(4,4-dimethylpiperidin-1-yl)-5′-fluoro-6-methyl-6′-(pentyloxy)-[3,3′-bipyridine]-5-yl]acetic acid (16.9 mg, 32% yield, 515.67% purity). observed ion=516.4. ¹H NMR (500 MHz, METHANOL-d4) δ 8.13 (s, 1H), 7.92 (s, 1H), 7.57 (br d, J=10.1 Hz, 1H), 5.77 (s, 1H), 4.51-4.43 (m, 2H), 2.78 (br s, 2H), 2.66 (s, 3H), 1.89-1.83 (m, 2H), 1.53-1.41 (m, 8H), 1.21 (s, 9H), 0.98 (t, J=7.0 Hz, 3H), 0.93 (s, 6H).

### Example 408

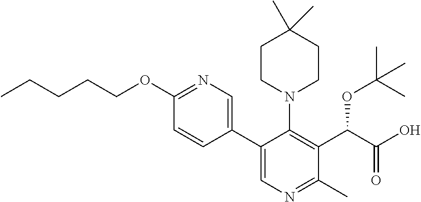 [SMILES: CCCCCOc1ccc(-c2cnc(C)c([C@H](OC(C)(C)C)C(=O)O)c2N2CCC(C)(C)CC2)cn1]

### (S)-2-(tert-Butoxy)-2-(4-(4, 4-dimethylpiperidin-1-yl)-6-methyl-6′-(pentyloxy)-[3, 3′-bipyridin]-5-yl)acetic acid

To a stirred solution of (S)-isopropyl 2-(tert-butoxy)-2-(4-(4,4-dimethylpiperidin-1-yl)-6-methyl-6′-(pentyloxy)-[3,3′-bipyridin]-5-yl)acetate (60 mg, 0.111 mmol) in methanol (15 mL) was added a solution of sodium hydroxide (133 mg, 3.33 mmol) in water (3.00 mL). The mixture was stirred overnight at 75° C. and pH was adjusted to 8. The mixture was concentrated and the residue was purified by Prep-HPLC to afford the desired product (S)-2-(tert-butoxy)-2-(4-(4,4-dimethylpiperidin-1-yl)-6-methyl-6′-(pentyloxy)-[3,3′-bipyridin]-5-yl)acetic acid (25 mg, 45.2% yield). LCMS [M+H]=498.2. ¹H NMR (400 MHz, CD3OD) δ 8.23 (s, 1H), 8.15 (d, J=2.0 Hz, 1H), 7.71 (dd, J=8.5, 2.4 Hz, 1H), 6.96 (d, J=8.6 Hz, 1H), 5.72 (s, 1H), 4.37 (qd, J=8.8, 3.9 Hz, 2H), 3.15 (s, 2H), 2.89 (s, 2H), 2.73 (s, 3H), 1.87-1.79 (m, 2H), 1.54-1.40 (m, 8H), 1.23 (s, 9H), 1.00-0.92 (m, 9H).

### Example 409

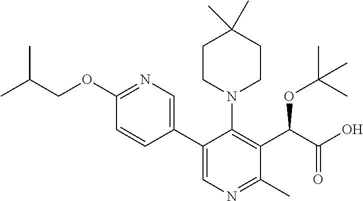 [SMILES: Cc1ncc(-c2ccc(OCC(C)C)nc2)c(N2CCC(C)(C)CC2)c1[C@@H](OC(C)(C)C)C(=O)O]

To a stirred solution of (S)-isopropyl 2-(tert-butoxy)-2-(4-(4,4-dimethylpiperidin-1-yl)-6′-isobutoxy-6-methyl-[3,3′-bipyridin]-5-yl)acetate (75 mg, 0.143 mmol) in methanol (10 mL) was added a solution of sodium hydroxide (5.71 mg, 0.143 mmol) in water (3.0 mL). The mixture was stirred overnight at 75° C. and pH was adjusted to 8. The mixture was concentrated and the residue purified by Prep-HPLC to afford the desired product (S)-2-(tert-butoxy)-2-(4-(4,4-dimethylpiperidin-1-yl)-6′-isobutoxy-6-methyl-[3,3′-bipyridin]-5-yl)acetic acid (40 mg, 58.0% yield) as a white solid. LCMS [M+H]=484.2. ¹H NMR (400 MHz, CD3OD) δ 8.31 (s, 1H), 8.17 (s, 1H), 7.73 (d, J=7.0 Hz, 1H), 7.00 (d, J=8.6 Hz, 1H), 5.68 (s, 1H), 4.20-4.13 (m, 2H), 3.13 (d, J=14.7 Hz, 2H), 2.95 (s, 2H), 2.79 (s, 3H), 2.13 (dt, J=13.4, 6.7 Hz, 1H), 1.49 (s, 4H), 1.25 (s, 9H), 1.06 (d, J=6.7 Hz, 6H), 0.97 (s, 6H).

### Example 410

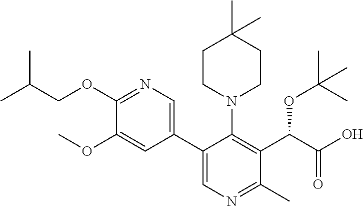 [SMILES: COc1cc(-c2cnc(C)c([C@H](OC(C)(C)C)C(=O)O)c2N2CCC(C)(C)CC2)cnc1OCC(C)C]

### (S)-2-(tert-Butoxy)-2-(4-(4,4-dimethylpiperidin-1-yl)-6′-isobutoxy-5′-methoxy-6-methyl-[3,3′-bipyridin]-5-yl)acetic acid

A mixture of sodium hydride (71.8 mg, 2.99 mmol) and 2-methylpropan-1-ol (148 mg, 1.993 mmol) in DMF (10 mL) was stirred over 30 mins. Then, (S)-isopropyl 2-(tert-butoxy)-2-(4-(4,4-dimethylpiperidin-1-yl)-6′-fluoro-5′-methoxy-6-methyl-[3,3′-bipyridin]-5-yl)acetate (100 mg, 0.199 mmol) was added and stirred at 25° C. After 4 h, the pH was of reaction mixture adjusted to 8, concentrated and purified by Prep-HPLC to afford the desired product (S)-2-(tert-butoxy)-2-(4-(4,4-dimethylpiperidin-1-yl)-6′-isobutoxy-5′-methoxy-6-methyl-[3,3′-bipyridin]-5-yl)acetic acid (25 mg, 23.19% yield) as a white solid. LCMS [M+H]=514.3. ¹H NMR (400 MHz, CD3OD) δ 8.13 (s, 1H), 7.64 (s, 1H), 7.24 (s, 1H), 5.77 (s, 1H), 4.21-4.14 (m, 2H), 3.92 (s, 3H), 3.15 (s, 2H), 2.82 (s, 2H), 2.66 (s, 3H), 2.19-2.13 (m, 1H), 1.47 (s, 4H), 1.21 (s, 9H), 1.06 (d, J=6.7 Hz, 6H), 0.92 (s, 6H).

### Example 411

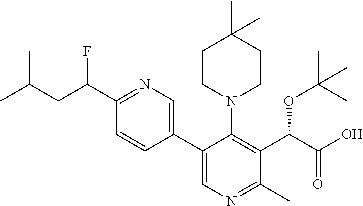 [SMILES: Cc1ncc(-c2ccc(C(F)CC(C)C)nc2)c(N2CCC(C)(C)CC2)c1[C@H](OC(C)(C)C)C(=O)O]

### (2S)-2-(tert-Butoxy)-2-(4-(4, 4-dimethylpiperidin-1-yl)-6′-(1-fluoro-3-methylbutyl)-6-methyl-[3, 3′-bipyridin]-5-yl)acetic acid

To a solution of (2S)-isopropyl 2-(tert-butoxy)-2-(4-(4,4-dimethylpiperidin-1-yl)-6′-(1-fluoro-3-methylbutyl)-6-methyl-[3,3′-bipyridin]-5-yl)acetate (410 mg, 0.757 mmol) in methanol (15 mL) was added a solution of sodium hydroxide (908 mg, 22.70 mmol) in water (5.00 mL). The mixture was stirred overnight at 75° C. Then, the pH was adjusted to 8 and concentrated. The residue was purified by Prep-HPLC to afford the desired product (2S)-2-(tert-butoxy)-2-(4-(4,4-dimethylpiperidin-1-yl)-6′-(1-fluoro-3-methylbutyl)-6-methyl-[3,3′-bipyridin]-5-yl)acetic acid (27.5 mg, 7.05% yield) as a white solid. LCMS [M+H]=500.3. ¹H NMR (400 MHz, CD3OD) δ 8.16-8.11 (m, 2H), 7.72 (dd, J=8.5, 2.4 Hz, 1H), 7.24 (d, J=8.0 Hz, 1H), 7.15 (d, J=6.8 Hz, 1H), 7.01 (t, J=8.3 Hz, 2H), 5.77 (s, 1H), 4.73 (ddd, J=15.9, 10.4, 3.7 Hz, 2H), 4.40-4.31 (m, 2H), 3.15 (s, 2H), 2.76 (s, 2H), 2.66 (s, 3H), 2.34 (s, 3H), 1.45 (s, 4H), 1.21 (s, 9H), 0.89 (s, 6H).

### Example 412

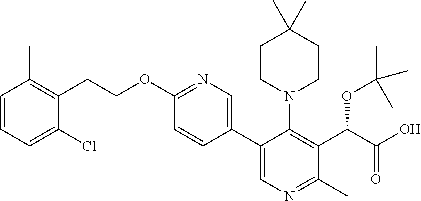 [SMILES: Cc1cccc(Cl)c1CCOc1ccc(-c2cnc(C)c([C@H](OC(C)(C)C)C(=O)O)c2N2CCC(C)(C)CC2)cn1]

### (S)-2-(tert-Butoxy)-2-(6′-(2-chloro-6-methylphenethoxy)-4-(4, 4-dimethylpiperidin-1-yl)-6-methyl-[3, 3′-bipyridin]-5-yl)acetic acid

To a pre-cooled solution of 2-(2-chloro-6-methylphenyl)ethanol (28.9 mg, 0.170 mmol) in DMF (4 mL) was added the NaH (67.8 mg, 1.696 mmol) in portions. Then, (S)-isopropyl 2-tert-butoxy-2-(4-(4,4-dimethylpiperidin-1-yl)-6′-hydroxy-6-methyl-3,3′-bipyridin-5-yl)acetate (80 mg, 0.170 mmol) in DMF (1 ml) was added quickly. The mixture was stirred at rt for 16 h and the pH of the reaction mixture was to 7 with acetic acid. The mixture was filtered and the filtrate was purified by Prep-HPLC (NH₄HCO₃ as buffer) to give (S)-2-(tert-butoxy)-2-(6′-(2-chloro-6-methylphenethoxy)-4-(4,4-dimethylpiperidin-1-yl)-6-methyl-[3,3′-bipyridin]-5-yl)acetic acid (21.8 mg, 0.038 mmol, 22.15% yield). LCMS [M+H]=580.0. ¹H NMR (400 MHz, CD3OD) δ 8.30 (s, 1H), 8.17 (s, 1H), 7.71 (d, J=8.1 Hz, 1H), 7.25 (d, J=7.6 Hz, 1H), 7.19-7.07 (m, 2H), 6.96 (d, J=8.6 Hz, 1H), 5.68 (s, 1H), 4.60 (t, J=7.1 Hz, 2H), 3.36 (d, J=7.2 Hz, 2H), 3.11 (s, 2H), 2.94 (s, 2H), 2.78 (s, 3H), 2.48 (s, 3H), 1.49 (s, 4H), 1.24 (s, 9H), 0.97 (s, 6H).

### Example 413

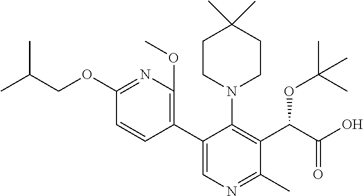 [SMILES: COc1nc(OCC(C)C)ccc1-c1cnc(C)c([C@H](OC(C)(C)C)C(=O)O)c1N1CCC(C)(C)CC1]

### (S)-2-(tert-Butoxy)-2-(4-(4, 4-dimethylpiperidin-1-yl)-6′-isobutoxy-2′-methoxy-6-methyl-[3,3′-bipyridin]-5-yl)acetic acid

To a solution of (S)-isopropyl 2-(tert-butoxy)-2-(4-(4,4-dimethylpiperidin-1-yl)-6′-isobutoxy-2′-methoxy-6-methyl-[3,3′-bipyridin]-5-yl)acetate (69 mg, 0.124 mmol) in methanol (3 mL) and H₂O (0.5 ml) was added sodium hydroxide (149 mg, 3.72 mmol). The mixture was stirred at 70° C. for 36 h, cooled, the pH of the mixture was adjusted to ˜7 and purified by Prep-HPLC to give (S)-2-(tert-butoxy)-2-(4-(4,4-dimethylpiperidin-1-yl)-6′-isobutoxy-2′-methoxy-6-methyl-[3,3′-bipyridin]-5-yl)acetic acid (20 mg, 0.039 mmol, 31.4% yield). LCMS [M+H]=514.0. ¹H NMR (400 MHz, CD3OD) δ 7.99 (d, J=42.3 Hz, 1H), 7.48 (dd, J=44.4, 8.0 Hz, 1H), 6.50 (t, J=8.0 Hz, 1H), 5.62 (d, J=51.5 Hz, 1H), 4.25-4.10 (m, 2H), 3.92 (d, J=5.3 Hz, 3H), 3.19 (s, 2H), 2.99 (s, 1H), 2.77 (s, 1H), 2.67 (s, 3H), 2.25-2.06 (m, 1H), 1.44 (s, 4H), 1.20 (d, J=11.5 Hz, 9H), 1.06 (d, J=6.6 Hz, 6H), 0.92 (d, J=17.6 Hz, 6H).

### Example 414

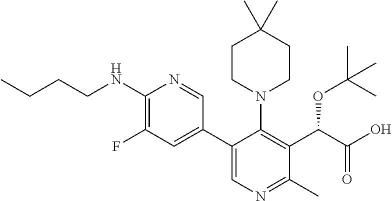 [SMILES: CCCCNc1ncc(-c2cnc(C)c([C@H](OC(C)(C)C)C(=O)O)c2N2CCC(C)(C)CC2)cc1F]

### (S)-2-(tert-Butoxy)-2-(6′-(butylamino)-4-(4, 4-dimethylpiperidin-1-yl)-5′-fluoro-6-methyl-[3,3′-bipyridin]-5-yl)acetic acid

To a solution of (S)-isopropyl 2-(tert-butoxy)-2-(6′-(butylamino)-4-(4,4-dimethylpiperidin-1-yl)-5′-fluoro-6-methyl-[3,3′-bipyridin]-5-yl)acetate (70 mg, 0.129 mmol) in methanol (8 mL) was added sodium hydroxide (103 mg, 2.58 mmol) in H₂O (3 mL). Then, the reaction mixture was stirred at 80° C. overnight. The mixture was acidified with aq. HCl to pH=7 at rt and concentrated. The crude was purified by Prep-HPLC to afford (S)-2-(tert-butoxy)-2-(6′-(butylamino)-4-(4,4-dimethylpiperidin-1-yl)-5′-fluoro-6-methyl-[3,3′-bipyridin]-5-yl)acetic acid (43.3 mg, 0.086 mmol, 67.1% yield) as a white solid. LCMS=501[M+H]. ¹H NMR (400 MHz, CD3OD): δ 8.31 (s, 1H), 7.81 (s, 1H), 7.41 (d, J=11.5 Hz, 1H), 5.67 (s, 1H), 3.60-3.42 (m, 2H), 3.15 (brs, 2H), 2.99 (brs, 2H), 2.78 (s, 3H), 1.69-1.67 (m, 2H), 1.55-1.48 (m, 6H), 1.24 (s, 9H), 1.09-0.88 (m, 9H).

### Example 415

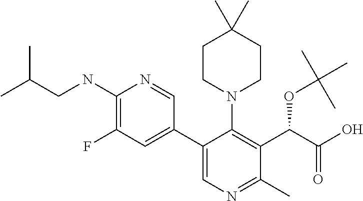 [SMILES: Cc1ncc(-c2cnc(NCC(C)C)c(F)c2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5′-fluoro-6′-(isobutylamino)-6-methyl-[3, 3′-bipyridin]-5-yl)acetic acid

To a solution of (S)-isopropyl 2-(tert-butoxy)-2-(4-(4,4-dimethylpiperidin-1-yl)-5′-fluoro-6′-(isobutylamino)-6-methyl-[3,3′-bipyridin]-5-yl)acetate (70 mg, 0.129 mmol) in methanol (8 mL) was added sodium hydroxide (103 mg, 2.58 mmol) in H₂O (3 mL). Then the reaction mixture was stirred at 80° C. overnight, cooled, acidified with aq. HCl to pH=7 and concentrated. The crude was purified by Prep-HPLC to afford (S)-2-(tert-butoxy)-2-(4-(4,4-dimethylpiperidin-1-yl)-5′-fluoro-6′-(isobutylamino)-6-methyl-[3,3′-bipyridin]-5-yl)acetic acid (45.5 mg, 0.091 mmol, 70.5% yield) as a white solid. LCMS=501.2 [M+H]. ¹H NMR (400 MHz, CD3OD): δ 8.31 (s, 1H), 7.80 (s, 1H), 7.41 (d, J=11.6 Hz, 1H), 5.67 (s, 1H), 3.36 (s, 1H), 3.30-3.26 (m, 2H), 3.18 (brs, 2H), 2.98 (brs, 2H), 2.78 (s, 3H), 2.08-1.89 (m, 1H), 1.55-1.48 (m, 4H), 1.24 (s, 9H), 1.00 (d, J=6.3 Hz, 12H).

### Example 416

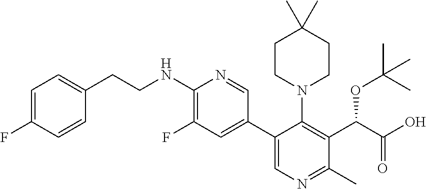 [SMILES: Cc1ncc(-c2cnc(NCCc3ccc(F)cc3)c(F)c2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5′-fluoro-6′-((4-fluorophenethyl)amino)-6-methyl-[3, 3′-bipyridin]-5-yl)acetic acid

To a solution of (S)-isopropyl 2-(tert-butoxy)-2-(4-(4,4-dimethylpiperidin-1-yl)-5′-fluoro-6′-((4-fluorophen-ethyl)amino)-6-methyl-[3,3′-bipyridin]-5-yl)acetate (80 mg, 0.131 mmol) in methanol (8 mL) was added sodium hydroxide (105 mg, 2.63 mmol) in H₂O (3 mL). Then, the reaction mixture was stirred at 80° C. overnight, cooled, acidified with aq. HCl to pH=7 and concentrated. The crude was purified by Prep-HPLC to afford (S)-2-(tert-butoxy)-2-(4-(4,4-dimethylpiperidin-1-yl)-5′-fluoro-6′-((4-fluorophenethyl)amino)-6-methyl-[3,3′-bipyridin]-5-yl)acetic acid (42.4 mg, 0.075 mmol, 56.9% yield) as a white solid. LCMS=567 [M+H]+. ¹H NMR (400 MHz, CD3OD) δ 8.30 (s, 1H), 7.84 (s, 1H), 7.37 (d, J=11.5 Hz, 1H), 7.33-7.22 (m, 2H), 7.10-6.93 (m, 2H), 5.67 (s, 1H), 3.73 (t, J=7.3 Hz, 2H), 3.18 (brs, 2H), 3.09-2.85 (m, 4H), 2.78 (s, 3H), 1.64-1.39 (m, 4H), 1.24 (s, 9H), 0.99 (s, 6H).

### Example 417

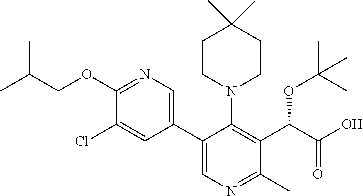 [SMILES: Cc1ncc(-c2cnc(OCC(C)C)c(Cl)c2)c(N2CCC(C)(C)CC2)c1[C@H](OC(C)(C)C)C(=O)O]

### (S)-2-(tert-Butoxy)-2-(5′-chloro-4-(4, 4-dimethylpiperidin-1-yl)-6′-isobutoxy-6-methyl-[3,3′-bipyridin]-5-yl)acetic acid

A mixture of (S)-isopropyl 2-(tert-butoxy)-2-(5′-chloro-4-(4,4-dimethylpiperidin-1-yl)-6′-isobutoxy-6-methyl-[3,3′-bipyridin]-5-yl)acetate (65 mg, 0.116 mmol) and sodium hydroxide (93 mg, 2.321 mmol) in methanol (6 mL) was heated at 80° C. overnight. The mixture was neutralized with aq. HCl to pH=7. The solvent was removed and the residue was purified by Prep-HPLC to give (S)-2-(tert-butoxy)-2-(5′-chloro-4-(4,4-dimethylpiperidin-1-yl)-6′-isobutoxy-6-methyl-[3,3′-bipyridin]-5-yl)acetic acid (45 mg, 0.087 mmol, 74.9% yield) as a white solid. LCMS=518 [M+H]. ¹H NMR (400 MHz, CD3OD): δ 8.34 (s, 1H), 8.11 (d, J=1.4 Hz, 1H), 7.90 (s, 1H), 5.67 (s, 1H), 4.26 (dd, J=6.5, 4.6 Hz, 2H), 3.14 (brs, 2H), 2.98 (brs, 2H), 2.78 (s, 3H), 2.27-2.12 (m, 1H), 1.55-148 (s, 4H), 1.24 (s, 9H), 1.09 (d, J=6.7 Hz, 6H), 0.98 (s, 6H).

### Example 418

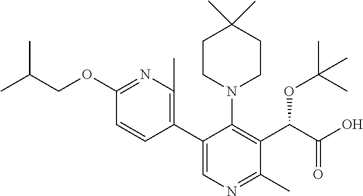 [SMILES: Cc1nc(OCC(C)C)ccc1-c1cnc(C)c([C@H](OC(C)(C)C)C(=O)O)c1N1CCC(C)(C)CC1]

### (S)-2-(tert-Butoxy)-2-(4-(4, 4-dimethylpiperidin-1-yl)-6′-isobutoxy-2′,6-dimethyl-[3, 3′-bipyridin]-5-yl)acetic acid

A mixture of (S)-isopropyl 2-(tert-butoxy)-2-(4-(4,4-dimethylpiperidin-1-yl)-6′-isobutoxy-2′,6-dimethyl-[3,3′-bipyridin]-5-yl)acetate (37 mg, 0.069 mmol) and sodium hydroxide (54.8 mg, 1.371 mmol) in methanol (3 mL) was heated at 80° C. overnight. The mixture was neutralized with aq. HCl to pH=7. The solvent was removed and the residue was purified by Prep-HPLC to give (S)-2-(tert-butoxy)-2-(4-(4,4-dimethylpiperidin-1-yl)-6′-isobutoxy-2′,6-dimethyl-[3,3′-bipyridin]-5-yl)acetic acid (22 mg, 0.044 mmol, 64.5% yield) as a white solid. LCMS=498 [M+H]. ¹H NMR (400 MHz, CD3OD): δ 8.31 (s, 0.7H), 8.14 (s, 0.4H), 7.53 (d, J=8.4 Hz, 1H), 6.81 (t, J=8.4 Hz, 1H), 5.68 (s, 0.8H), 5.57 (s, 0.4H), 4.23-4.00 (m, 2H), 3.25-2.84 (m, 4H), 2.77 (d, J=5.6 Hz, 3H), 2.35 (s, 2H), 2.24 (s, 1H), 2.15-2.10 (m, 1H), 1.55-1.33 (m, 4H), 1.25 (d, J=3.0 Hz, 9H), 1.06 (dd, J=6.7, 2.3 Hz, 6H), 0.95 (d, J=4.8 Hz, 6H).

### Example 419

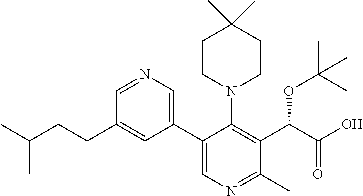 [SMILES: Cc1ncc(-c2cncc(CCC(C)C)c2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5′-isopentyl-6-methyl-[3, 3′-bipyridin]-5-yl)acetic acid

A mixture of (S)-isopropyl 2-(tert-butoxy)-2-(4-(4,4-dimethylpiperidin-1-yl)-5′-isopentyl-6-methyl-[3,3′-bipyridin]-5-yl)acetate (70 mg, 0.134 mmol) and sodium hydroxide (107 mg, 2.67 mmol) in methanol (3 mL) was heated at 80° C. overnight. The mixture was neutralized with aq. HCl to pH=7. The solvent was removed and the residue was purified by Prep-HPLC to give (S)-2-(tert-butoxy)-2-(4-(4,4-dimethylpiperidin-1-yl)-5′-isopentyl-6-methyl-[3,3′-bipyridin]-5-yl)acetic acid (27 mg, 0.056 mmol, 41.8% yield) as a white solid. LCMS=482 [M+H]⁺. ¹H NMR (400 MHz, CD3OD): δ 8.63 (s, 1H), 8.52 (s, 1H), 8.38 (s, 1H), 7.88 (s, 1H), 5.67 (s, 1H), 3.20-3.02 (m, 4H), 2.89-2.68 (m, 5H), 1.81-1.56 (m, 3H), 1.47 (brs, 4H), 1.24 (s, 9H), 1.01 (dd, J=6.2, 1.1 Hz, 6H), 0.96 (s, 6H).

### Example 420

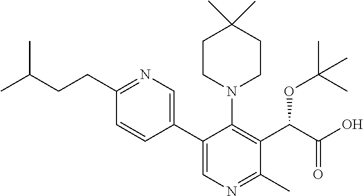 [SMILES: Cc1ncc(-c2ccc(CCC(C)C)nc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-6′-isopentyl-6-methyl-[3, 3′-bipyridin]-5-yl)acetic acid

A mixture of (S)-isopropyl 2-(tert-butoxy)-2-(4-(4,4-dimethylpiperidin-1-yl)-6′-isopentyl-6-methyl-[3,3′-bipyridin]-5-yl)acetate (75 mg, 0.143 mmol) and sodium hydroxide (115 mg, 2.86 mmol) in methanol (3 mL) was heated at 80° C. overnight. The mixture was neutralized with aq. HCl to pH=7. The solvent was removed and the residue was purified by Prep-HPLC to give (S)-2-(tert-butoxy)-2-(4-(4,4-dimethylpiperidin-1-yl)-6′-isopentyl-6-methyl-[3,3′-bipyridin]-5-yl)acetic acid (34.8 mg, 0.072 mmol, 50.5% yield) as a white solid. LCMS=482 [M+H]. ¹H NMR (400 MHz, CD3OD): δ 8.63 (s, 1H), 8.40 (s, 1H), 8.02 (s, 1H), 7.70 (s, 1H), 5.66 (s, 1H), 3.12-2.80 (m, 6H), 2.81 (s, 3H), 1.82-1.54 (m, 3H), 1.43 (brs, 4H), 1.24 (s, 9H), 1.10-0.96 (m, 12H).

### Example 421

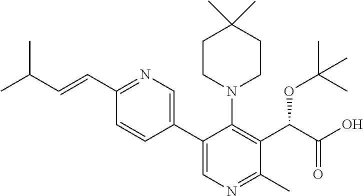 [SMILES: Cc1ncc(-c2ccc(/C=C/C(C)C)nc2)c(N2CCC(C)(C)CC2)c1[C@H](OC(C)(C)C)C(=O)O]

### (S,E)-2-(tert-Butoxy)-2-(4-(4, 4-dimethylpiperidin-1-yl)-6-methyl-6′-(3-methylbut-1-en-1-yl)-[3, 3′-bipyridin]-5-yl)acetic acid

A mixture of (S,E)-isopropyl 2-(tert-butoxy)-2-(4-(4,4-dimethylpiperidin-1-yl)-6-methyl-6′-(3-methylbut-1-en-1-yl)-[3,3′-bipyridin]-5-yl)acetate (30 mg, 0.058 mmol) and sodium hydroxide (46.0 mg, 1.150 mmol) in methanol (3 mL) was heated at 80° C. overnight. The mixture was neutralized with aq. HCl to pH=7. The solvent was removed and the residue was purified by Prep-HPLC to give (S,E)-2-(tert-butoxy)-2-(4-(4,4-dimethylpiperidin-1-yl)-6-methyl-6′-(3-methylbut-1-en-1-yl)-[3,3′-bipyridin]-5-yl)acetic acid (9.6 mg, 0.020 mmol, 34.8% yield) as a white solid. LCMS=480 [M+H]. ¹H NMR (400 MHz, CD3OD): δ 8.56 (s, 1H), 8.38 (s, 1H), 7.92 (s, 1H), 7.79 (d, J=8.2 Hz, 1H), 7.07-6.80 (m, 1H), 6.61 (d, J=16.0 Hz, 1H), 5.66 (s, 1H), 3.15 (brs, 2H), 3.05 (brs, 2H), 2.80 (s, 3H), 2.63-2.61 (m, 1H), 1.55-1.48 (m, 4H), 1.25 (s, 9H), 1.19 (d, J=6.8 Hz, 6H), 0.97 (s, 6H).

### Example 422

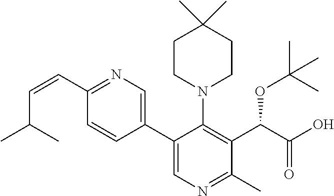 [SMILES: Cc1ncc(-c2ccc(/C=C\C(C)C)nc2)c(N2CCC(C)(C)CC2)c1[C@H](OC(C)(C)C)C(=O)O]

### (S,Z)-2-(tert-Butoxy)-2-(4-(4, 4-dimethylpiperidin-1-yl)-6-methyl-6′-(3-methylbut-1-en-1-yl)-[3, 3′-bipyridin]-5-yl)acetic acid

A mixture of (S,Z)-isopropyl 2-(tert-butoxy)-2-(4-(4,4-dimethylpiperidin-1-yl)-6-methyl-6′-(3-methylbut-1-en-1-yl)-[3,3′-bipyridin]-5-yl)acetate (70 mg, 0.134 mmol) and sodium hydroxide (107 mg, 2.68 mmol) in methanol (3 mL) was heated at 80° C. overnight. Then, the mixture was neutralized with aq. HCl to pH=7. The solvent was removed and the residue was purified by Prep-HPLC to give (S,Z)-2-(tert-butoxy)-2-(4-(4,4-dimethylpiperidin-1-yl)-6-methyl-6′-(3-methylbut-1-en-1-yl)-[3,3′-bipyridin]-5-yl)acetic acid (38.6 mg, 0.079 mmol, 58.9% yield) as a white solid. LCMS=480 [M+H]. ¹H NMR (400 MHz, CD3OD): δ 8.65 (s, 1H), 8.40 (s, 1H), 7.94 (s, 1H), 7.62 (s, 1H), 6.47 (d, J=11.8 Hz, 1H), 5.93-5.89 (m, 1H), 5.67 (s, 1H), 3.41 (brs, 1H), 3.10-2.95 (m, 4H), 2.81 (s, 3H), 1.48 (brs, 4H), 1.23 (s, 9H), 1.11 (dd, J=6.5, 4.1 Hz, 6H), 0.97 (s, 6H).

### Example 423

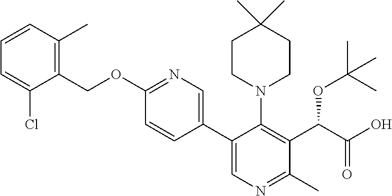 [SMILES: Cc1cccc(Cl)c1COc1ccc(-c2cnc(C)c([C@H](OC(C)(C)C)C(=O)O)c2N2CCC(C)(C)CC2)cn1]

### (S)-2-(tert-Butoxy)-2-(6′-((2-chloro-6-methylbenzyl)oxy)-4-(4, 4-dimethylpiperidin-1-yl)-6-methyl-[3, 3′-bipyridin]-5-yl)acetic acid

To a solution of (S)-isopropyl 2-(tert-butoxy)-2-(6′-((2-chloro-6-methylbenzyl)oxy)-4-(4,4-dimethylpiperidin-1-yl)-6-methyl-[3,3′-bipyridin]-5-yl)acetate (70 mg, 0.115 mmol) in ethanol (3 mL) was added a solution of sodium hydroxide (46.0 mg, 1.151 mmol) in H₂O (0.3 mL) and heated at 90° C. for 16 hr. Then, the pH was adjusted with acetic acid and purified by preparative HPLC (NH₄CO₃ as buffer) to give (S)-2-(tert-butoxy)-2-(6′-((2-chloro-6-methylbenzyl)oxy)-4-(4,4-dimethylpiperidin-1-yl)-6-methyl-[3,3′-bipyridin]-5-yl)acetic acid (21.2 mg, 0.037 mmol, 32.4% yield) as a white solid. LCMS [M+H]=565.27. ¹H NMR (500 MHz, DMSO-d₆): ¹H NMR (400 MHz, DMSO) δ 8.18 (s, 1H), 8.10 (s, 1H), 7.71 (d, J=8.4 Hz, 1H), 7.43-7.22 (m, 3H), 6.95 (d, J=8.4 Hz, 1H), 5.82 (s, 1H), 5.56 (d, J=11.6 Hz, 1H), 5.46 (d, J=11.6 Hz, 1H), 3.34 (s, 3H), 2.96 (s, 1H), 2.42 (s, 3H), 2.22 (s, 2H), 1.56 (s, 1H), 1.32 (s, 2H), 1.13 (s, 10H), 0.82 (d, J=62.4 Hz, 6H).

### Example 424

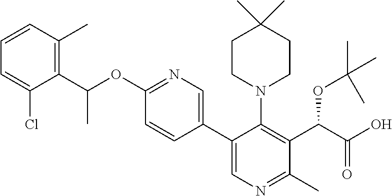 [SMILES: Cc1cccc(Cl)c1C(C)Oc1ccc(-c2cnc(C)c([C@H](OC(C)(C)C)C(=O)O)c2N2CCC(C)(C)CC2)cn1]

### (2S)-2-(tert-Butoxy)-2-(6′-(1-(2-chloro-6-methylphenyl)ethoxy)-4-(4, 4-dimethylpiperidin-1-yl)-6-methyl-[3, 3′-bipyridin]-5-yl)acetic acid

To a pre-cooled solution of 1-(2-chloro-6-methylphenyl)ethanol (145 mg, 0.848 mmol) in DMF (4 mL) was added the NaH (33.9 mg, 0.848 mmol) in portion. Then, (S)-isopropyl 2-(tert-butoxy)-2-(4-(4,4-dimethylpiperidin-1-yl)-6′-fluoro-6-methyl-[3,3′-bipyridin]-5-yl)acetate (80 mg, 0.170 mmol) in DMF (1 ML) was added rapidly and stirred at rt. After 16 h, the pH was adjusted to about 7 with acetic acid, the mixture filtered and purified by Prep-HPLC to provide (2S)-2-(tert-butoxy)-2-(6′-(1-(2-chloro-6-methylphenyl)ethoxy)-4-(4,4-dimethylpiperidin-1-yl)-6-methyl-[3,3′-bipyridin]-5-yl)acetic acid (21.6 mg, 21.6% yield) as white solid. LCMS [M+H]=580 and 582. ¹H NMR (400 MHz, CD3OD) δ 8.11-7.97 (m, 2H), 7.64 (dt, J=8.5, 2.5 Hz, 1H), 7.20 (dd, J=7.5, 1.5 Hz, 1H), 7.11-7.03 (m, 2H), 6.95 (d, J=8.5 Hz, 1H), 6.72 (tt, J=6.8, 3.4 Hz, 1H), 5.73 (d, J=3.4 Hz, 1H), 3.05 (s, 2H), 2.80 (s, 2H), 2.64 (s, 3H), 2.57 (d, J=3.2 Hz, 3H), 1.75 (dd, J=6.9, 1.9 Hz, 3H), 1.36 (d, J=11.3 Hz, 4H), 1.18 (s, 9H), 0.87 (d, J=15.0 Hz, 3H), 0.80 (s, 3H).

### Example 425

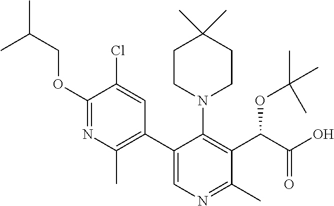 [SMILES: Cc1nc(OCC(C)C)c(Cl)cc1-c1cnc(C)c([C@H](OC(C)(C)C)C(=O)O)c1N1CCC(C)(C)CC1]

### (S)-2-(tert-Butoxy)-2-(5′-chloro-4-(4, 4-dimethylpiperidin-1-yl)-6′-isobutoxy-2′,6-dimethyl-[3, 3′-bipyridin]-5-yl)acetic acid

To a solution of (S)-isopropyl 2-(tert-butoxy)-2-(5′-chloro-4-(4,4-dimethylpiperidin-1-yl)-6′-isobutoxy-2′,6-dimethyl-[3,3′-bipyridin]-5-yl)acetate (20 mg, 0.035 mmol) in DMF (2 mL) was added a solution of NaOH (13.93 mg, 0.348 mmol) in H₂O (0.5 mL. The mixture was stirred at room temperature for 72 hr. The pH value of the reaction mixture was adjusted to ˜7 by with aq. HCl (1 M) and purified by preparative HPLC (NH₄CO₃ as buffer) to (S)-2-(tert-butoxy)-2-(5′-chloro-4-(4,4-dimethylpiperidin-1-yl)-6′-isobutoxy-2′,6-dimethyl-[3,3′-bipyridin]-5-yl)acetic acid (2 mg, 3.62 μmol, 10.40% yield) as a white solid. LCMS [M+H]=532. ¹H NMR (400 MHz, CD3OD) δ 8.08 (s, 1H), 7.56 (s, 1H), 5.81 (s, 1H), 4.80-4.46 (m, 2H), 4.33-4.13 (m, 2H), 2.98 (s, 2H), 2.75 (s, 1H), 2.69-2.61 (m, 4H), 2.30 (s, 2H), 2.23-2.08 (m, 2H), 1.42 (s, 4H), 1.22 (d, J=1.3 Hz, 9H), 1.08 (dd, J=6.7, 2.9 Hz, 6H), 0.90 (d, J=3.4 Hz, 6H).

### Example 426

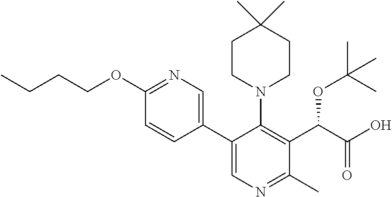 [SMILES: CCCCOc1ccc(-c2cnc(C)c([C@H](OC(C)(C)C)C(=O)O)c2N2CCC(C)(C)CC2)cn1]

### (S)-2-tert-Butoxy-2-(6′-butoxy-4-(4, 4-dimethylpiperidin-1-yl)-6-methyl-3, 3′-bipyridin-5-yl)acetic acid

To a solution of (S)-isopropyl 2-tert-butoxy-2-(6′-butoxy-4-(4,4-dimethylpiperidin-1-yl)-6-methyl-3,3′-bipyridin-5-yl)acetate (55 mg, 0.105 mmol) in methanol (3 mL) and water (0.5 ml) was added the sodium hydroxide (42 mg, 1.05 mmol) and heated at 70° C. overnight. Then, the reaction mixture was neutralized with acetic acid and purified by preparative HPLC (TFA as buffer) to give (S)-2-tert-butoxy-2-(6′-butoxy-4-(4,4-dimethylpiperidin-1-yl)-6-methyl-3,3′-bipyridin-5-yl)acetic acid (30 mg, 59%). LCMS [M+H]=484.3. ¹H NMR (400 MHz, CD3OD) δ 8.32 (s, 1H), 8.17 (s, 1H), 7.72 (d, J=6.8 Hz, 1H), 6.98 (d, J=8.5 Hz, 1H), 5.68 (s, 1H), 4.47-4.29 (m, 2H), 3.11 (s, 2H), 2.95 (s, 2H), 2.79 (s, 3H), 1.81 (dt, J=14.4, 6.6 Hz, 2H), 1.59-1.36 (m, 6H), 1.25 (s, 9H), 1.05-0.90 (m, 9H).

### Example 427

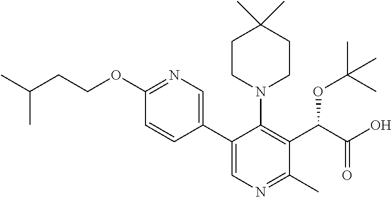 [SMILES: Cc1ncc(-c2ccc(OCCC(C)C)nc2)c(N2CCC(C)(C)CC2)c1[C@H](OC(C)(C)C)C(=O)O]

### (S)-2-tert-Butoxy-2-(4-(4, 4-dimethylpiperidin-1-yl)-6′-(isopentyloxy)-6-methyl-3, 3′-bipyridin-5-yl)acetic acid

To an ice-cold solution of 3-methylbutan-1-ol (74.8 mg, 0.848 mmol) in anhydrous DMF (3 mL) was added sodium hydride (33.9 mg, 0.848 mmol). The mixture was stirred at rt for 20 min. Then, (S)-isopropyl 2-tert-butoxy-2-(4-(4,4-dimethylpiperidin-1-yl)-6′-fluoro-6-methyl-3,3′-bipyridin-5-yl)acetate (80 mg, 0.170 mmol) was added and stirred at 90° C. overnight. Then, cooled, neutralized and purified by preparative HPLC (TFA as buffer) to give (S)-2-tert-butoxy-2-(4-(4,4-dimethylpiperidin-1-yl)-6′-(isopentyloxy)-6-methyl-3,3′-bipyridin-5-yl)acetic acid (35 mg, 41%). LCMS [M+H]=498.4. ¹H NMR (400 MHz, CD3OD) δ 8.31 (s, 1H), 8.17 (s, 1H), 7.72 (d, J=8.4 Hz, 1H), 6.98 (t, J=7.8 Hz, 1H), 5.68 (s, 1H), 4.42 (dq, J=6.7, 4.0 Hz, 2H), 3.12 (s, 2H), 2.95 (s, 2H), 2.77 (d, J=7.1 Hz, 3H), 1.85 (dt, J=13.4, 6.7 Hz, 1H), 1.72 (q, J=6.7 Hz, 2H), 1.48 (d, J=10.7 Hz, 4H), 1.24 (s, 9H), 1.04-0.90 (m, 12H).

### Example 428

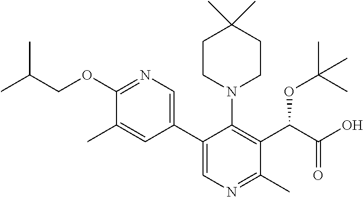 [SMILES: Cc1cc(-c2cnc(C)c([C@H](OC(C)(C)C)C(=O)O)c2N2CCC(C)(C)CC2)cnc1OCC(C)C]

### (S)-2-tert-Butoxy-2-(4-(4, 4-dimethylpiperidin-1-yl)-6′-isobutoxy-5′,6-dimethyl-3, 3′-bipyridin-5-yl)acetic acid

To a ice-cold solution of 2-methylpropan-1-ol (198 mg, 2.68 mmol) in anhydrous THF (1 mL) was added the sodium hydride (107 mg, 2.68 mmol). The mixture was stirred at rt for 30 min. Then, (S)-isopropyl 2-tert-butoxy-2-(4-(4,4-dimethylpiperidin-1-yl)-6′-fluoro-5′,6-dimethyl-3,3′-bipyridin-5-yl)acetate (70 mg, 0.148 mmol) was added and stirred at 70° C. overnight. Then, reaction mixture acidified with aq. HCl and purified by preparative HPLC (TFA as buffer) to give (S)-2-tert-butoxy-2-(4-(4,4-dimethylpiperidin-1-yl)-6′-isobutoxy-5′,6-dimethyl-3,3′-bipyridin-5-yl)acetic acid (55 mg, 58%). LCMS [M+H]=498.3. ¹H NMR (400 MHz, CD3OD) δ 8.30 (s, 1H), 7.98 (s, 1H), 7.56 (s, 1H), 5.68 (s, 1H), 4.26-4.10 (m, 2H), 3.13 (s, 2H), 2.95 (s, 2H), 2.78 (s, 3H), 2.31 (s, 3H), 2.16 (dt, J=13.3, 6.7 Hz, 1H), 1.58-1.40 (m, 4H), 1.24 (s, 9H), 1.08 (d, J=6.7 Hz, 6H), 0.97 (s, 6H).

### Example 429

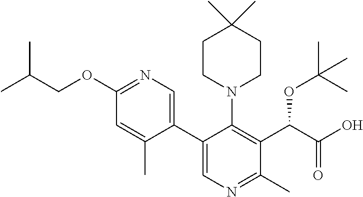 [SMILES: Cc1cc(OCC(C)C)ncc1-c1cnc(C)c([C@H](OC(C)(C)C)C(=O)O)c1N1CCC(C)(C)CC1]

### (2S)-2-tert-Butoxy-2-(4-(4, 4-dimethylpiperidin-1-yl)-6′-isobutoxy-4′,6-dimethyl-3, 3′-bipyridin-5-yl)acetic acid

To an ice-cold solution of 2-methylpropan-1-ol (275 mg, 3.71 mmol) in anhydrous THF (4 mL) was added sodium hydride (148 mg, 3.71 mmol). The mixture was stirred at rt for 30 min. Then, (2S)-isopropyl 2-tert-butoxy-2-(4-(4,4-dimethylpiperidin-1-yl)-6′-fluoro-4′,6-dimethyl-3,3′-bipyridin-5-yl)acetate (180 mg, 0.371 mmol) was added and stirred at 70° C. overnight. Then, the reaction mixture was acidified with aq. HCl and purified by preparative HPLC (TFA as buffer) to give (2S)-2-tert-butoxy-2-(4-(4,4-dimethylpiperidin-1-yl)-6′-isobutoxy-4′,6-dimethyl-3,3′-bipyridin-5-yl)acetic acid (50 mg, 27%). LCMS [M+H]=498.3. ¹H NMR (400 MHz, CD3OD) δ 8.24 (d, J=60.7 Hz, 1H), 7.97 (d, J=47.7 Hz, 1H), 6.88 (d, J=13.5 Hz, 1H), 5.62 (d, J=43.4 Hz, 1H), 4.20-4.05 (m, 2H), 3.22-2.86 (m, 4H), 2.78 (s, 3H), 2.23 (s, 2H), 2.12 (dd, J=8.4, 4.9 Hz, 2H), 1.43 (s, 4H), 1.25 (d, J=1.8 Hz, 9H), 1.05 (dd, J=6.7, 3.3 Hz, 6H), 0.94 (d, J=8.3 Hz, 6H).

### Example 430

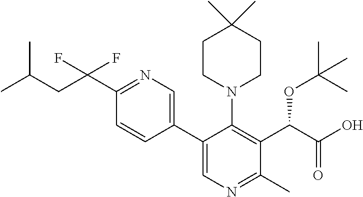 [SMILES: Cc1ncc(-c2ccc(C(F)(F)CC(C)C)nc2)c(N2CCC(C)(C)CC2)c1[C@H](OC(C)(C)C)C(=O)O]

### (S)-2-tert-Butoxy-2-(6′-(1,1-difluoro-3-methylbutyl)-4-(4, 4-dimethylpiperidin-1-yl)-6-methyl-3, 3′-bipyridin-5-yl)acetic acid

To a solution of (S)-isopropyl 2-tert-butoxy-2-(6′-(1,1-difluoro-3-methylbutyl)-4-(4,4-dimethylpiperidin-1-yl)-6-methyl-3,3′-bipyridin-5-yl)acetate (70 mg, 0.125 mmol) in methanol (3 mL) and water (0.5 ml) was added the sodium hydroxide (50 mg, 1.25 mmol) and heated at 70° C. overnight. Then, the pH was adjusted to neutral and purified by preparative HPLC (TFA as buffer) to give (S)-2-tert-butoxy-2-(6′-(1,1-difluoro-3-methylbutyl)-4-(4,4-dimethylpiperidin-1-yl)-6-methyl-3,3′-bipyridin-5-yl)acetic acid (50 mg, 77%). LCMS [M+H]=518.3. ¹H NMR (400 MHz, CD3OD) δ 8.66 (s, 1H), 8.17 (s, 1H), 7.99 (dd, J=8.0, 2.0 Hz, 1H), 7.92-7.80 (m, 1H), 5.84 (s, 1H), 3.01 (s, 2H), 2.76 (t, J=26.6 Hz, 2H), 2.66 (s, 3H), 2.30 (tdd, J=17.1, 6.4, 4.2 Hz, 2H), 1.81 (dt, J=13.3, 6.7 Hz, 1H), 1.40 (d, J=21.1 Hz, 4H), 1.26-1.16 (m, 9H), 0.97 (dd, J=6.7, 2.3 Hz, 6H), 0.89 (s, 6H).

### Example 431

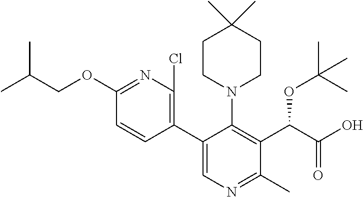 [SMILES: Cc1ncc(-c2ccc(OCC(C)C)nc2Cl)c(N2CCC(C)(C)CC2)c1[C@H](OC(C)(C)C)C(=O)O]

### (2S)-2-tert-Butoxy-2-(2′-chloro-4-(4, 4-dimethylpiperidin-1-yl)-6′-isobutoxy-6-methyl-3, 3′-bipyridin-5-yl)acetic acid

To a solution of (2S)-isopropyl 2-tert-butoxy-2-(2′-chloro-4-(4,4-dimethylpiperidin-1-yl)-6′-isobutoxy-6-methyl-3,3′-bipyridin-5-yl)acetate (100 mg, 0.179 mmol) in methanol (3 mL) and water (0.5 ml) was added the sodium hydroxide (72 mg, 1.79 mmol) and heated at 70° C. overnight. Then, cooled, adjusted pH to neutral with acetic acid and purified by preparative HPLC (TFA as buffer) to give (2S)-2-tert-butoxy-2-(2′-chloro-4-(4,4-dimethylpiperidin-1-yl)-6′-isobutoxy-6-methyl-3,3′-bipyridin-5-yl)acetic acid (20 mg, 21%). LCMS [M+H]=518.2. ¹H NMR (400 MHz, CD3OD) δ 7.99 (d, J=38.6 Hz, 1H), 7.59 (dd, J=40.7, 8.3 Hz, 1H), 6.82 (dd, J=8.3, 7.1 Hz, 1H), 5.59 (d, J=65.0 Hz, 1H), 4.08-3.97 (m, 2H), 3.06-2.89 (m, 2H), 2.75 (s, 2H), 2.63-2.52 (m, 3H), 2.07-1.94 (m, 1H), 1.32 (d, J=6.1 Hz, 3H), 1.20 (d, J=9.1 Hz, 1H), 1.14-1.04 (m, 9H), 0.94 (dd, J=6.7, 2.8 Hz, 6H), 0.85-0.74 (m, 6H).

### Example 432 Example 433

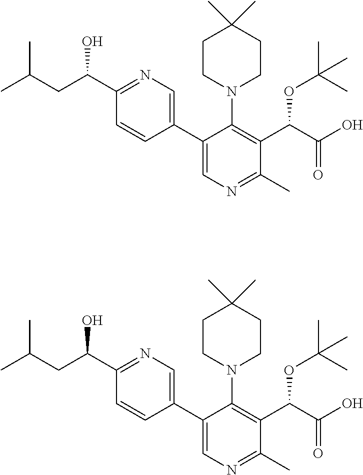

### (S)-2-tert-Butoxy-2-(4-(4, 4-dimethylpiperidin-1-yl)-6′-((S)-1-hydroxy-3-methylbutyl)-6-methyl-3, 3′-bipyridin-5-yl)acetic acid

To a solution of (2S)-isopropyl 2-tert-butoxy-2-(4-(4,4-dimethylpiperidin-1-yl)-6′-(1-hydroxy-3-methylbutyl)-6-methyl-3,3′-bipyridin-5-yl)acetate (250 mg, 0.463 mmol) in methanol (3 mL) and water (0.5 ml) was added the sodium hydroxide (185 mg, 4.63 mmol). Then, the solution was heated at 70° C. overnight, cooled, adjusted pH to neutral with acetic acid and purified by preparative HPLC (TFA as buffer) to give (S)-2-tert-butoxy-2-(4-(4,4-dimethylpiperidin-1-yl)-6′-((S)-1-hydroxy-3-methylbutyl)-6-methyl-3,3′-bipyridin-5-yl)acetic acid (5 mg, 2%). LCMS [M+H]=498.4. ¹H NMR (400 MHz, CD3OD) δ 8.48 (s, 1H), 8.13 (s, 1H), 7.87 (dd, J=8.1, 1.7 Hz, 1H), 7.72 (d, J=8.1 Hz, 1H), 5.81 (s, 1H), 4.87 (d, J=5.6 Hz, 2H), 3.06 (d, J=7.3 Hz, 2H), 2.73 (d, J=10.9 Hz, 2H), 2.66 (s, 3H), 1.87-1.73 (m, 1H), 1.72-1.60 (m, 2H), 1.40 (d, J=22.5 Hz, 4H), 1.21 (s, 9H), 1.00 (dd, J=8.1, 6.7 Hz, 6H), 0.88 (s, 6H).

Also isolated

(S)-2-tert-butoxy-2-(4-(4,4-dimethylpiperidin-1-yl)-6′-((R)-1-hydroxy-3-methylbutyl)-6-methyl-3,3′-bipyridin-5-yl)acetic acid (5 mg, 2%)). LCMS [M+H]=498.4. ¹H NMR (400 MHz, CD3OD) δ 8.48 (d, J=1.6 Hz, 1H), 8.13 (s, 1H), 7.86 (dd, J=8.1, 2.0 Hz, 1H), 7.72 (d, J=8.1 Hz, 1H), 5.82 (s, 1H), 4.89-4.85 (m, 2H), 3.18-2.90 (m, 2H), 2.74 (dd, J=33.7, 15.7 Hz, 2H), 2.66 (s, 3H), 1.90-1.74 (m, 1H), 1.74-1.58 (m, 2H), 1.40 (d, J=22.3 Hz, 4H), 1.21 (s, 9H), 1.00 (dd, J=11.3, 6.6 Hz, 6H), 0.89 (s, 6H).

### Example 434

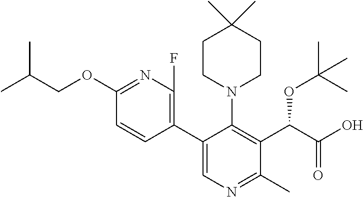 [SMILES: Cc1ncc(-c2ccc(OCC(C)C)nc2F)c(N2CCC(C)(C)CC2)c1[C@H](OC(C)(C)C)C(=O)O]

### (2S)-2-tert-Butoxy-2-(4-(4, 4-dimethylpiperidin-1-yl)-2′-fluoro-6′-isobutoxy-6-methyl-3, 3′-bipyridin-5-yl)acetic acid

To a solution of (2S)-isopropyl 2-tert-butoxy-2-(4-(4,4-dimethylpiperidin-1-yl)-2′-fluoro-6′-isobutoxy-6-methyl-3,3′-bipyridin-5-yl)acetate (51 mg, 0.094 mmol) in methanol (2 mL) and water (0.5 ml) was added sodium hydroxide (38 mg, 0.94 mmol). Then, the solution was heated at 70° C. overnight cooled, adjusted to neutral with acidic acid and purified by preparative HPLC (TFA as buffer) to give (2S)-2-tert-butoxy-2-(4-(4,4-dimethylpiperidin-1-yl)-2′-fluoro-6′-isobutoxy-6-methyl-3,3′-bipyridin-5-yl)acetic acid (14 mg, 30%). LCMS [M+H]=502.1. ¹H NMR (400 MHz, CD3OD) δ 8.09 (d, J=26.9 Hz, 1H), 7.74 (d, J=40.1 Hz, 1H), 6.86 (d, J=8.1 Hz, 1H), 5.79 (d, J=36.1 Hz, 1H), 4.19-4.03 (m, 2H), 3.06 (d, J=38.1 Hz, 2H), 2.91 (d, J=19.5 Hz, 1H), 2.79 (d, J=14.2 Hz, 1H), 2.65 (s, 3H), 2.13 (dt, J=13.4, 6.7 Hz, 1H), 1.45 (d, J=3.5 Hz, 4H), 1.21 (s, 9H), 1.06 (d, J=6.7 Hz, 6H), 0.91 (s, 6H).

### Example 435

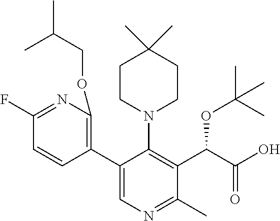 [SMILES: Cc1ncc(-c2ccc(F)nc2OCC(C)C)c(N2CCC(C)(C)CC2)c1[C@H](OC(C)(C)C)C(=O)O]

### (2S)-2-tert-Butoxy-2-(4-(4, 4-dimethylpiperidin-1-yl)-6′-fluoro-2′-isobutoxy-6-methyl-3, 3′-bipyridin-5-yl)acetic acid

To a solution of (2S)-isopropyl 2-tert-butoxy-2-(4-(4,4-dimethylpiperidin-1-yl)-6′-fluoro-2′-isobutoxy-6-methyl-3,3′-bipyridin-5-yl)acetate (30 mg, 0.055 mmol) in methanol (2 mL) and water (0.5 ml) was added sodium hydroxide (22 mg, 0.55 mmol) and heated at 70° C. overnight. The solution was then acidified and purified by preparative HPLC (TFA as buffer) to give (2S)-2-tert-butoxy-2-(4-(4,4-dimethylpiperidin-1-yl)-6′-fluoro-2′-isobutoxy-6-methyl-3,3′-bipyridin-5-yl)acetic acid (4 mg, 12%). LCMS [M+H]=502.1. ¹H NMR (400 MHz, CD3OD) δ 7.89 (d, J=41.2 Hz, 1H), 7.68-7.53 (m, 1H), 6.62 (dt, J=9.8, 4.9 Hz, 1H), 5.69 (s, 1H), 4.03-3.90 (m, 2H), 2.81 (s, 2H), 2.65 (s, 2H), 2.53 (s, 3H), 1.81 (dt, J=13.1, 6.7 Hz, 1H), 1.30 (s, 4H), 1.08 (s, 9H), 0.84-0.67 (m, 12H).

### Example 436

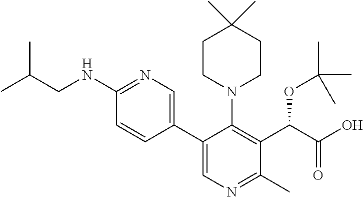 [SMILES: Cc1ncc(-c2ccc(NCC(C)C)nc2)c(N2CCC(C)(C)CC2)c1[C@H](OC(C)(C)C)C(=O)O]

### (S)-2-tert-Butoxy-2-(4-(4, 4-dimethylpiperidin-1-yl)-6′-(isobutylamino)-6-methyl-3, 3′-bipyridin-5-yl)acetic acid

To a solution of (S)-isopropyl 2-tert-butoxy-2-(4-(4,4-dimethylpiperidin-1-yl)-6′-(isobutylamino)-6-methyl-3,3′-bipyridin-5-yl)acetate (55 mg, 0.105 mmol) in methanol (5 ml) and water (1 ml) was added NaOH (62.9 mg, 1.572 mmol). The mixture was stirred at 100° C. for 16 hours. The mixture was then acidified with acetic acid (adjust solution to pH=7), concentrated and purified by preparative HPLC (TFA as buffer) to give (S)-2-tert-butoxy-2-(4-(4,4-dimethylpiperidin-1-yl)-6′-(isobutylamino)-6-methyl-3,3′-bipyridin-5-yl)acetic acid (25 mg, 49.4%). LCMS [M+H]=483.3. ¹H NMR (400 MHz, CD3OD) δ 8.19 (s, 1H), 7.84 (s, 1H), 7.67 (d, J=9.2 Hz, 1H), 7.02 (d, J=9.2 Hz, 1H), 5.50 (s, 1H), 4.74 (s, 2H), 3.10 (dd, J=11.1, 9.2 Hz, 4H), 3.01-2.77 (m, 2H), 2.62 (s, 3H), 1.88 (dp, J=13.5, 6.8 Hz, 1H), 1.51-1.27 (m, 4H), 1.09 (s, 9H), 0.91 (t, J=8.4 Hz, 6H), 0.85 (s, 6H).

### Example 437

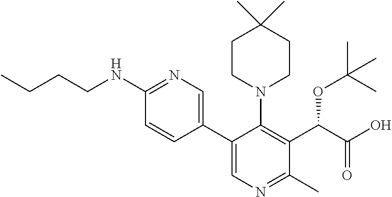 [SMILES: CCCCNc1ccc(-c2cnc(C)c([C@H](OC(C)(C)C)C(=O)O)c2N2CCC(C)(C)CC2)cn1]

### (S)-2-tert-Butoxy-2-(6′-(butylamino)-4-(4, 4-dimethylpiperidin-1-yl)-6-methyl-3, 3′-bipyridin-5-yl)acetic acid

To a solution of (S)-isopropyl 2-(tert-butoxy)-2-(6′-(butylamino)-4-(4,4-dimethylpiperidin-1-yl)-6-methyl-[3,3′-bipyridin]-5-yl)acetate (55 mg, 0.105 mmol) in methanol (5 ml) and water (1 ml) was added NaOH (41.9 mg, 1.048 mmol) and stirred at 100° C. for 16 hours. Then, the reaction mixture was acidified with acetic acid (adjusted solution to pH=7), concentrated and purified by preparative HPLC (TFA as buffer) to give (S)-2-tert-butoxy-2-(6′-(butylamino)-4-(4,4-dimethylpiperidin-1-yl)-6-methyl-3,3′-bipyridin-5-yl)acetic acid (25 mg, 49.4%). LCMS [M+H]=483.2. ¹H NMR (400 MHz, CD3OD) δ 8.32 (s, 1H), 7.98 (s, 1H), 7.80 (s, 1H), 7.11 (s, 1H), 5.67 (s, 1H), 3.45 (dd, J=22.1, 14.9 Hz, 4H), 3.15 (s, 3H), 3.04 (s, 3H), 2.76 (s, 4H), 1.83-1.66 (m, 2H), 1.52 (dd, J=14.9, 7.6 Hz, 6H), 1.24 (s, 10H), 1.03 (dd, J=13.4, 5.9 Hz, 10H).

### Example 438

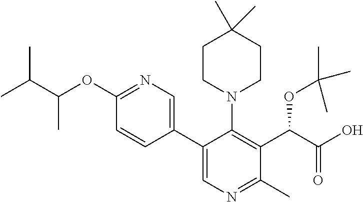 [SMILES: Cc1ncc(-c2ccc(OC(C)C(C)C)nc2)c(N2CCC(C)(C)CC2)c1[C@H](OC(C)(C)C)C(=O)O]

### (2S)-2-tert-Butoxy-2-(4-(4, 4-dimethylpiperidin-1-yl)-6-methyl-6′-(3-methylbutan-2-yloxy)-3, 3′-bipyridin-5-yl)acetic acid

To a solution of 3-methylbutan-2-ol (187 mg, 2.120 mmol) in anhydrous DMF (5 ml) was added NaH (127 mg, 3.18 mmol) and the resulting mixture was stirred at 100° C. for 30 min. Then, (S)-isopropyl 2-tert-butoxy-2-(4-(4,4-dimethylpiperidin-1-yl)-6′-fluoro-6-methyl-3,3′-bipyridin-5-yl)acetate (100 mg, 0.212 mmol) was added to the mixture and stirred at 100° C. for 16 hours. The reaction mixture was diluted with water (20 mL), extracted with EtOAc (2×40 mL) and the combined organic layers were washed with brine (20 mL), dried over Na₂SO₄ and concentrated to afford crude product. The crude product was dissolved in MeOH and purified by Prep-HPLC to afford (2S)-2-tert-butoxy-2-(4-(4,4-dimethylpiperidin-1-yl)-6-methyl-6′-methylbutan-2-yloxy)-3,3′-bipyridin-5-yl)acetic acid (25 mg, 23.69%). LCMS [M+H]=497.3. ¹H NMR (400 MHz, CD3OD) δ 8.33 (d, J=2.7 Hz, 1H), 8.16 (s, 1H), 7.71 (d, J=8.2 Hz, 1H), 6.94 (d, J=8.6 Hz, 1H), 5.68 (s, 1H), 5.12 (tt, J=12.1, 6.1 Hz, 1H), 3.12 (s, 2H), 2.91 (d, J=46.5 Hz, 2H), 2.79 (s, 3H), 2.13-1.87 (m, 1H), 1.46 (t, J=17.1 Hz, 4H), 1.30 (dt, J=8.9, 4.5 Hz, 3H), 1.25 (s, 8H), 1.13-0.85 (m, 11H).

### Example 439

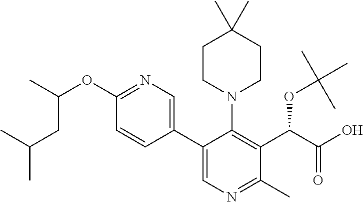 [SMILES: Cc1ncc(-c2ccc(OC(C)CC(C)C)nc2)c(N2CCC(C)(C)CC2)c1[C@H](OC(C)(C)C)C(=O)O]

### (2S)-2-tert-Butoxy-2-(4-(4, 4-dimethylpiperidin-1-yl)-6-methyl-6′-(4-methylpentan-2-yloxy)-3, 3′-bipyridin-5-yl)acetic acid

To a solution of 4-methylpentan-2-ol (217 mg, 2.120 mmol) in anhydrous DMF (5 ml) was added NaH (127 mg, 3.18 mmol) and the resulting mixture was stirred at 100° C. for 30 min. Then, (S)-isopropyl 2-tert-butoxy-2-(4-(4,4-dimethylpiperidin-1-yl)-6′-fluoro-6-methyl-3,3′-bipyridin-5-yl)acetate (100 mg, 0.212 mmol) added and stirred at 100° C. for 16 hours. To the reaction mixture was added water (20 ml), extracted with EtOAc (2×40 mL) and the combined organic layers were washed with brine (20 mL), dried over Na₂SO₄ and concentrated to afford crude product. The crude product was dissolved in MeOH, and the solution was purified by Prep-HPLC to afford (2S)-2-tert-butoxy-2-(4-(4,4-dimethylpiperidin-1-yl)-6-methyl-6′-(4-methylpentan-2-yloxy)-3,3′-bipyridin-5-yl)acetic acid (40 mg, 36.9%). LCMS [M+H]=511.3. ¹H NMR (400 MHz, CD3OD) δ 8.33 (s, 1H), 8.17 (s, 1H), 7.71 (d, J=8.5 Hz, 1H), 6.92 (d, J=8.5 Hz, 1H), 5.68 (s, 1H), 5.42 (dt, J=13.1, 8.2 Hz, 1H), 3.11 (s, 2H), 2.92 (d, J=32.9 Hz, 2H), 2.79 (s, 3H), 1.87-1.68 (m, 2H), 1.45 (dd, J=12.3, 6.3 Hz, 4H), 1.41 (d, J=5.6 Hz, 1H), 1.34 (dd, J=6.0, 4.7 Hz, 3H), 1.25 (s, 7H), 1.06-0.84 (m, 10H).

### Example 440

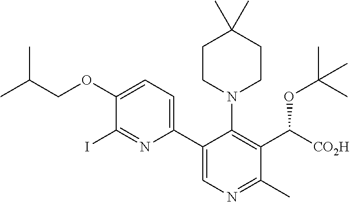 [SMILES: Cc1ncc(-c2ccc(OCC(C)C)c(I)n2)c(N2CCC(C)(C)CC2)c1[C@H](OC(C)(C)C)C(=O)O]

### (S)-2-tert-Butoxy-2-(4′-(4, 4-dimethylpiperidin-1-yl)-6-iodo-5-isobutoxy-6′-methyl-2, 3′-bipyridin-5′-yl)acetic acid

To a solution of (S)-isopropyl 2-tert-butoxy-2-(4′-(4,4-dimethylpiperidin-1-yl)-6-iodo-5-isobutoxy-6′-methyl-2,3′-bipyridin-5′-yl)acetate (150 mg, 0.230 mmol) in MeOH (10 mL) and water (2 mL) was added NaOH (27.6 mg, 0.691 mmol) and stirred for 20 hours at 100° C. The mixture was concentrated under vacuo, then, HCl (1N, 0.5 mL) was added. The mixture was extracted by EtOAc (5 ml) and concentrated to give crude product. The crude product was purified by HPLC to give (S)-2-tert-butoxy-2-(4′-(4,4-dimethylpiperidin-1-yl)-6-iodo-5-isobutoxy-6′-methyl-2,3′-bipyridin-5′-yl)acetic acid (53 mg, 37.8% yield). ESI-MS (EI⁺, m/z)⁺[M+H]⁺ 610.2. ¹H NMR (400 MHz, MeOD) δ 8.20 (s, 1H), 7.48 (d, J=8.3 Hz, 1H), 7.37 (d, J=8.3 Hz, 1H), 5.87 (s, 1H), 4.02-3.89 (m, 2H), 2.93-2.86 (m, 4H), 2.65 (s, 3H), 2.19 (td, J=13.0, 6.4 Hz, 1H), 1.48-1.42 (m, 4H), 1.21 (s, 9H), 1.16 (d, J=6.7 Hz, 6H), 0.96 (d, J=17.1 Hz, 6H).

### Example 441

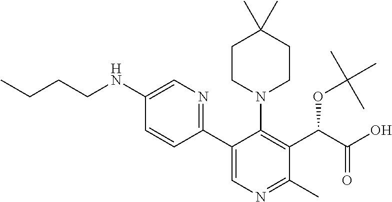 [SMILES: CCCCNc1ccc(-c2cnc(C)c([C@H](OC(C)(C)C)C(=O)O)c2N2CCC(C)(C)CC2)nc1]

### (S)-2-(tert-Butoxy)-2-(5-(butylamino)-4′-(4, 4-dimethylpiperidin-1-yl)-6′-methyl-[2, 3′-bipyridin]-5′-yl)acetic acid

To a solution of (S)-isopropyl 2-(tert-butoxy)-2-(5-(butylamino)-4′-(4,4-dimethylpiperidin-1-yl)-6′-methyl-[2,3′-bipyridin]-5′-yl)acetate (65 mg, 0.124 mmol) in methanol (8 mL) was added sodium hydroxide (99 mg, 2.477 mmol) in H₂O (3 mL). Then, the reaction mixture was stirred at 80° C. overnight. The mixture was acidified with HCl to pH=7. The crude was purified by Prep-HPLC to afford (S)-2-(tert-butoxy)-2-(5-(butylamino)-4′-(4,4-dimethylpiperidin-1-yl)-6′-methyl-[2,3′-bipyridin]-5′-yl)acetic acid (28 mg, 0.057 mmol, 46.2% yield) as a white solid. LCMS [M+H]=483.

### Example 442

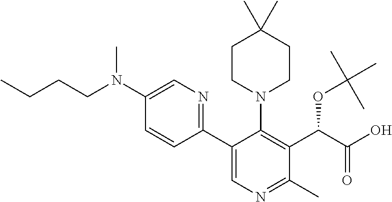 [SMILES: CCCCN(C)c1ccc(-c2cnc(C)c([C@H](OC(C)(C)C)C(=O)O)c2N2CCC(C)(C)CC2)nc1]

### (S)-2-(tert-Butoxy)-2-(5-(butyl(methyl)amino)-4′-(4, 4-dimethylpiperidin-1-yl)-6′-methyl-[2, 3′-bipyridin]-5′-yl)acetic acid

To a solution of (S)-isopropyl 2-(tert-butoxy)-2-(5-(butyl(methyl)amino)-4′-(4,4-dimethylpiperidin-1-yl)-6′-methyl-[2,3′-bipyridin]-5′-yl)acetate (50 mg, 0.093 mmol) in methanol (8 mL) was added sodium hydroxide (74.2 mg, 1.856 mmol) in H₂O (3 mL). Then, the reaction mixture was stirred at 80° C. overnight. The mixture was acidified with HCl to pH=7. The crude was purified by Prep-HPLC to afford (S)-2-(tert-butoxy)-2-(5-(butyl(methyl)amino)-4′-(4,4-dimethylpiperidin-1-yl)-6′-methyl-[2,3′-bipyridin]-5′-yl)acetic acid (24.1 mg, 0.049 mmol, 52.3% yield) as a white solid. LCMS [M+H]=497.

The following compounds could be prepared by procedures similar to those described in this specification for the Examples above.

### Example 443

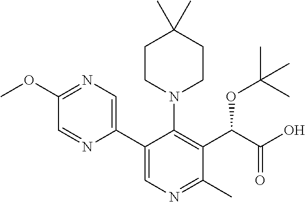 [SMILES: COc1cnc(-c2cnc(C)c([C@H](OC(C)(C)C)C(=O)O)c2N2CCC(C)(C)CC2)cn1]

### (S)-2-(tert-Butoxy)-2-(4-(4, 4-dimethylpiperidin-1-yl)-5-(5-methoxypyrazin-2-yl)-2-methylpyridin-3-yl)acetic acid

### Example 444

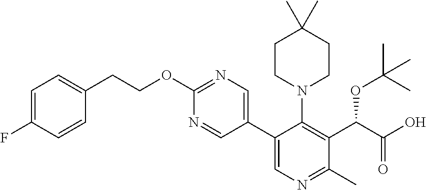 [SMILES: Cc1ncc(-c2cnc(OCCc3ccc(F)cc3)nc2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(2-(4-fluorophenethoxy)pyrimidin-5-yl)-2-methylpyridin-3-yl)acetic acid

### Example 445

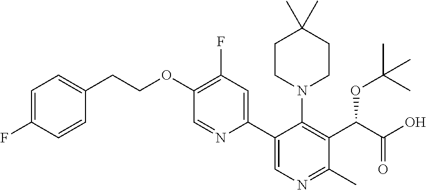 [SMILES: Cc1ncc(-c2cc(F)c(OCCc3ccc(F)cc3)cn2)c(N2CCC(C)(C)CC2)c1[C@H](OC(C)(C)C)C(=O)O]

### (S)-2-(tert-Butoxy)-2-(4′-(4, 4-dimethylpiperidin-1-yl)-4-fluoro-5-(4-fluorophenethoxy)-6′-methyl-[2, 3′-bipyridin]-5′-yl)acetic acid

### Example 446

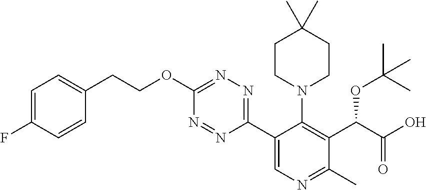 [SMILES: Cc1ncc(-c2nnc(OCCc3ccc(F)cc3)nn2)c(N2CCC(C)(C)CC2)c1[C@H](OC(C)(C)C)C(=O)O]

### (S)-2-(tert-Butoxy)-2-(4-(4, 4-dimethylpiperidin-1-yl)-5-(6-(4-fluorophenethoxy)-1, 2,4,5-tetrazin-3-yl)-2-methylpyridin-3-yl)acetic acid

### Example 447

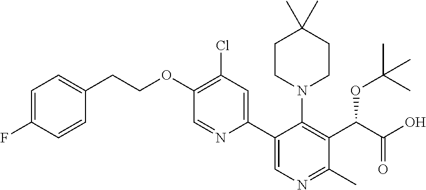 [SMILES: Cc1ncc(-c2cc(Cl)c(OCCc3ccc(F)cc3)cn2)c(N2CCC(C)(C)CC2)c1[C@H](OC(C)(C)C)C(=O)O]

### (S)-2-(tert-Butoxy)-2-(4-chloro-4′-(4, 4-dimethylpiperidin-1-yl)-5-(4-fluorophenethoxy)-6′-methyl-[2, 3′-bipyridin]-5′-yl)acetic acid

### Example 448

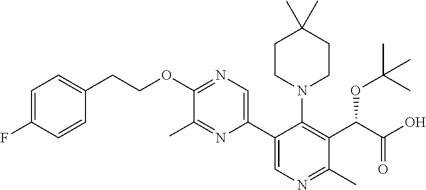 [SMILES: Cc1nc(-c2cnc(C)c([C@H](OC(C)(C)C)C(=O)O)c2N2CCC(C)(C)CC2)cnc1OCCc1ccc(F)cc1]

### (S)-2-(tert-Butoxy)-2-(4-(4, 4-dimethylpiperidin-1-yl)-5-(5-(4-fluorophenethoxy)-6-methylpyrazin-2-yl)-2-methylpyridin-3-yl)acetic acid

### Example 449

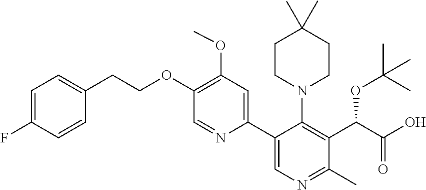 [SMILES: COc1cc(-c2cnc(C)c([C@H](OC(C)(C)C)C(=O)O)c2N2CCC(C)(C)CC2)ncc1OCCc1ccc(F)cc1]

### (S)-2-(tert-Butoxy)-2-(4′-(4, 4-dimethylpiperidin-1-yl)-5-(4-fluorophenethoxy)-4-methoxy-6′-methyl-[2, 3′-bipyridin]-5′-yl)acetic acid

### Example 450

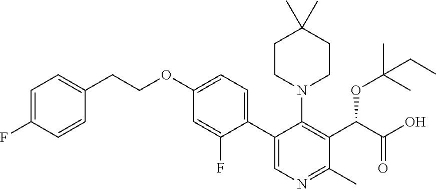 [SMILES: CCC(C)(C)O[C@H](C(=O)O)c1c(C)ncc(-c2ccc(OCCc3ccc(F)cc3)cc2F)c1N1CCC(C)(C)CC1]

### (S)-2-(4-(4, 4-Dimethylpiperidin-1-yl)-5-(2-fluoro-4-(4-fluorophenethoxy)phenyl)-2-methylpyridin-3-yl)-2-(tert-pentyloxy)acetic acid

### Example 451

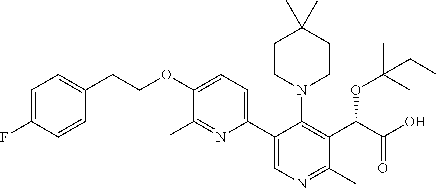 [SMILES: CCC(C)(C)O[C@H](C(=O)O)c1c(C)ncc(-c2ccc(OCCc3ccc(F)cc3)c(C)n2)c1N1CCC(C)(C)CC1]

### (S)-2-(4′-(4, 4-Dimethylpiperidin-1-yl)-5-(4-fluorophenethoxy)-6, 6′-dimethyl-[2, 3′-bipyridin]-5′-yl)-2-(tert-pentyloxy)acetic acid

### Example 452

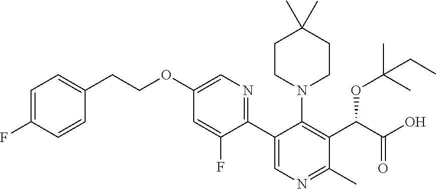 [SMILES: CCC(C)(C)O[C@H](C(=O)O)c1c(C)ncc(-c2ncc(OCCc3ccc(F)cc3)cc2F)c1N1CCC(C)(C)CC1]

### (S)-2-(4′-(4, 4-Dimethylpiperidin-1-yl)-3-fluoro-5-(4-fluorophenethoxy)-6′-methyl-[2, 3′-bipyridin]-5′-yl)-2-(tert-pentyloxy)acetic acid

### Example 453

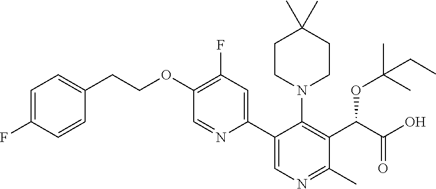 [SMILES: CCC(C)(C)O[C@H](C(=O)O)c1c(C)ncc(-c2cc(F)c(OCCc3ccc(F)cc3)cn2)c1N1CCC(C)(C)CC1]

### (S)-2-(4′-(4, 4-Dimethylpiperidin-1-yl)-4-fluoro-5-(4-fluorophenethoxy)-6′-methyl-[2, 3′-bipyridin]-5′-yl)-2-(tert-pentyloxy)acetic acid

### Example 454

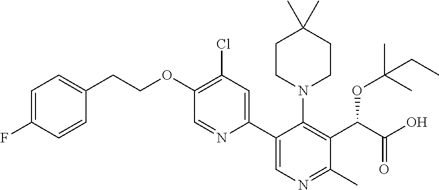 [SMILES: CCC(C)(C)O[C@H](C(=O)O)c1c(C)ncc(-c2cc(Cl)c(OCCc3ccc(F)cc3)cn2)c1N1CCC(C)(C)CC1]

### (S)-2-(4-Chloro-4′-(4, 4-dimethylpiperidin-1-yl)-5-(4-fluorophenethoxy)-6′-methyl-[2, 3′-bipyridin]-5′-yl)-2-(tert-pentyloxy)acetic acid

### Example 455

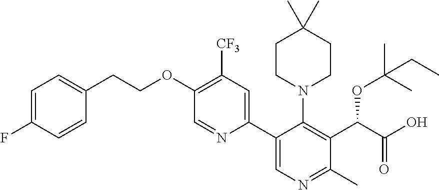 [SMILES: CCC(C)(C)O[C@H](C(=O)O)c1c(C)ncc(-c2cc(C(F)(F)F)c(OCCc3ccc(F)cc3)cn2)c1N1CCC(C)(C)CC1]

### (S)-2-(4′-(4, 4-Dimethylpiperidin-1-yl)-5-(4-fluorophenethoxy)-6′-methyl-4-(trifluoromethyl)-[2, 3′-bipyridin]-5′-yl)-2-(tert-pentyloxy)acetic acid

### Example 456

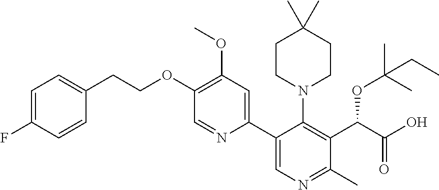 [SMILES: CCC(C)(C)O[C@H](C(=O)O)c1c(C)ncc(-c2cc(OC)c(OCCc3ccc(F)cc3)cn2)c1N1CCC(C)(C)CC1]

### (S)-2-(4′-(4, 4-Dimethylpiperidin-1-yl)-5-(4-fluorophenethoxy)-4-methoxy-6′-methyl-[2, 3′-bipyridin]-5′-yl)-2-(tert-pentyloxy)acetic acid

### Example 457

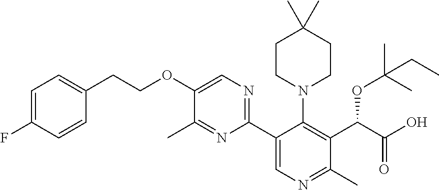 [SMILES: CCC(C)(C)O[C@H](C(=O)O)c1c(C)ncc(-c2ncc(OCCc3ccc(F)cc3)c(C)n2)c1N1CCC(C)(C)CC1]

### (S)-2-(4-(4, 4-Dimethylpiperidin-1-yl)-5-(5-(4-fluorophenethoxy)-4-methylpyrimidin-2-yl)-2-methylpyridin-3-yl)-2-(tert-pentyloxy)acetic acid

### Example 458

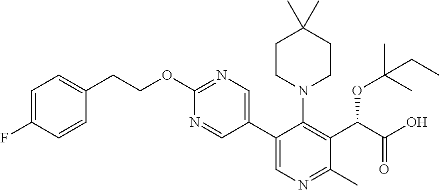 [SMILES: CCC(C)(C)O[C@H](C(=O)O)c1c(C)ncc(-c2cnc(OCCc3ccc(F)cc3)nc2)c1N1CCC(C)(C)CC1]

### (S)-2-(4-(4, 4-Dimethylpiperidin-1-yl)-5-(2-(4-fluorophenethoxy)pyrimidin-5-yl)-2-methylpyridin-3-yl)-2-(tert-pentyloxy)acetic acid

### Example 459

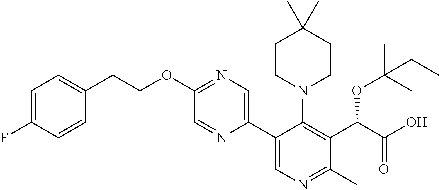 [SMILES: CCC(C)(C)O[C@H](C(=O)O)c1c(C)ncc(-c2cnc(OCCc3ccc(F)cc3)cn2)c1N1CCC(C)(C)CC1]

### (S)-2-(4-(4, 4-Dimethylpiperidin-1-yl)-5-(5-(4-fluorophenethoxy)pyrazin-2-yl)-2-methylpyridin-3-yl)-2-(tert-pentyloxy)acetic acid

### Example 460

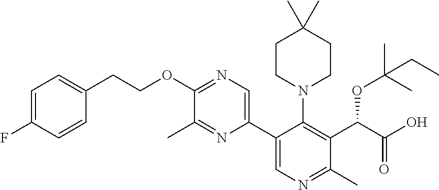 [SMILES: CCC(C)(C)O[C@H](C(=O)O)c1c(C)ncc(-c2cnc(OCCc3ccc(F)cc3)c(C)n2)c1N1CCC(C)(C)CC1]

### (S)-2-(4-(4, 4-Dimethylpiperidin-1-yl)-5-(5-(4-fluorophenethoxy)-6-methylpyrazin-2-yl)-2-methylpyridin-3-yl)-2-(tert-pentyloxy)acetic acid

### Example 461

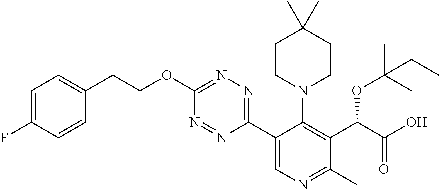 [SMILES: CCC(C)(C)O[C@H](C(=O)O)c1c(C)ncc(-c2nnc(OCCc3ccc(F)cc3)nn2)c1N1CCC(C)(C)CC1]

### (S)-2-(4-(4, 4-Dimethylpiperidin-1-yl)-5-(6-(4-fluorophenethoxy)-1,2,4,5-tetrazin-3-yl)-2-methylpyridin-3-yl)-2-(tert-pentyloxy)acetic acid

### Example 462

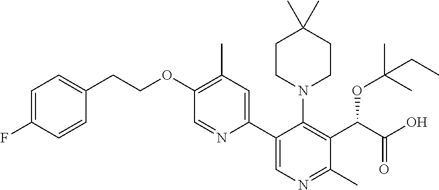 [SMILES: CCC(C)(C)O[C@H](C(=O)O)c1c(C)ncc(-c2cc(C)c(OCCc3ccc(F)cc3)cn2)c1N1CCC(C)(C)CC1]

### (S)-2-(4′-(4, 4-Dimethylpiperidin-1-yl)-5-(4-fluorophenethoxy)-4, 6′-dimethyl-[2, 3′-bipyridin]-5′-yl)-2-(tert-pentyloxy)acetic acid

### Example 463

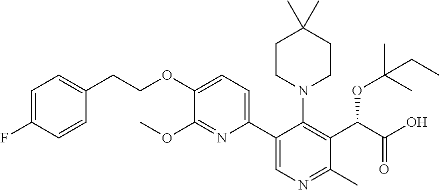 [SMILES: CCC(C)(C)O[C@H](C(=O)O)c1c(C)ncc(-c2ccc(OCCc3ccc(F)cc3)c(OC)n2)c1N1CCC(C)(C)CC1]

### (S)-2-(4′-(4, 4-Dimethylpiperidin-1-yl)-5-(4-fluorophenethoxy)-6-methoxy-6′-methyl-[2, 3′-bipyridin]-5′-yl)-2-(tert-pentyloxy)acetic acid

### Example 464

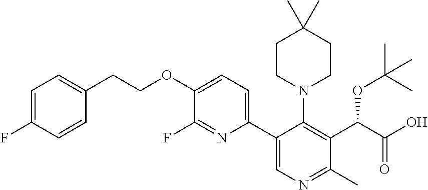 [SMILES: Cc1ncc(-c2ccc(OCCc3ccc(F)cc3)c(F)n2)c(N2CCC(C)(C)CC2)c1[C@H](OC(C)(C)C)C(=O)O]

### (S)-2-(tert-Butoxy)-2-(4′-(4, 4-dimethylpiperidin-1-yl)-6-fluoro-5-(4-fluorophenethoxy)-6′-methyl-[2, 3′-bipyridin]-5′-yl)acetic acid

### Example 465

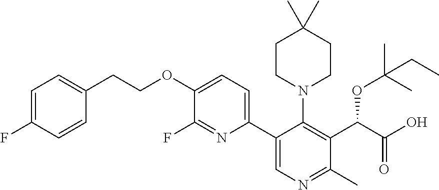 [SMILES: CCC(C)(C)O[C@H](C(=O)O)c1c(C)ncc(-c2ccc(OCCc3ccc(F)cc3)c(F)n2)c1N1CCC(C)(C)CC1]

### (S)-2-(4′-(4, 4-Dimethylpiperidin-1-yl)-6-fluoro-5-(4-fluorophenethoxy)-6′-methyl-[2, 3′-bipyridin]-5′-yl)-2-(tert-pentyloxy)acetic acid

### Biological Methods

Inhibition of HIV Replication:

A recombinant NL-RLuc proviral clone was constructed in which a section of the nef gene form NL4-3 was replaced with the Renilla Luciferase gene. This virus is fully infectious and can undergo multiple cycles of replication in cell culture. In addition, the luciferous reporter provides a simple and easy method for quantitating the extent of virus growth and consequently, the antiviral activity of test compounds. The plasmid pNLRLuc contains the proviral NL-Rluc DNA cloned into pUC18 at the PvuII site. The NL-RLuc virus was prepared by transfection of 293T cells with the plasmid pNLRLuc. Transfections were performed using the LipofectAMINE PLUS kit form Invitrogen (Carlsbad, Calif.) according to the manufacturer and the virus generated was titered in MT-2 cells. For susceptibility analyses, the titrated virus was used to infect MT-2 cells in the presence of compound, and after 5 days of incubation, cells were processed and quantitated for virus growth by the amount of expressed luciferase. Assay media was RPMI 1640 supplemented with 10% heat inactivated fetal bovine serum (FBS), 100 units/ml penicillin G/100 units/ml streptomycin, 10 mM HEPES buffer pH 7.55 and 2 mM L-glutamine. The results form at least 2 experiments were used to calculate the EC₅₀ values. Luciferase was quantitated using the Dual Luciferase kit form Promega (Madison, Wis.). Susceptibility of viruses to compounds was determined by incubation in the presence of serial dilutions of the compound. The 50% effective concentration (EC₅₀) was calculated by using the exponential form of the median effect equation where (Fa)=1/[1+(ED₅₀/drug conc.)^{m}](Johnson V A, Byington R T. Infectivity Assay. In Techniques in HIV Research. ed. Aldovini A, Walker B D. 71-76. New York: Stockton Press. 1990). Results are summarized in Table 1.

TABLE 1

| Example | EC₅₀ μM |
|---|---|
| 1 | 0.020 |
| 2 | 0.005 |
| 3 | 0.008 |
| 4 | 0.008 |
| 5 | 0.007 |
| 6 | 0.014 |
| 7 | 0.002 |
| 8 | 0.003 |
| 9 | 0.38 |
| 10 | 0.031 |
| 11 | 0.004 |
| 12 | 0.004 |
| 13 | 0.010 |
| 14 | 0.002 |
| 15 | 0.016 |
| 16 | 0.002 |
| 17 | 0.085 |
| 18 | 0.004 |
| 19 | 0.004 |
| 20 | 0.002 |
| 21 | 0.002 |
| 22 | 0.003 |
| 23 | 0.002 |
| 24 | 0.002 |
| 25 | 0.0008 |
| 26 | 0.0005 |
| 27 | 0.0007 |
| 28 | 0.0007 |
| 29 | 0.0002 |
| 30 | 0.002 |
| 31 | 0.0008 |
| 32 | 0.005 |
| 33 | 0.002 |
| 34 | 0.0007 |
| 35 | 0.001 |
| 36 | 0.004 |
| 37 | 0.002 |
| 38 | 0.008 |
| 39 | 0.003 |
| 40 | 0.002 |
| 41 | 0.004 |
| 42 | 0.003 |
| 43 | 0.0006 |
| 44 | 0.003 |
| 45 | 0.004 |
| 46 | 0.004 |
| 47 | 0.004 |
| 48 | 0.003 |
| 49 | 0.0007 |
| 50 | 0.002 |
| 51 | 0.005 |
| 52 | 0.001 |
| 53 | 0.003 |
| 54 | 0.002 |
| 55 | 0.003 |
| 56 | 0.002 |
| 57 | 0.013 |
| 58 | 0.018 |
| 59 | 0.003 |
| 60 | 0.025 |
| 61 | 0.011 |
| 62 | 0.003 |
| 63 | 0.006 |
| 64 | 0.005 |
| 65 | 0.002 |
| 66 | 0.002 |
| 67 | 0.027 |
| 68 | 0.002 |
| 69 | 0.003 |
| 70 | 0.002 |
| 71 | 0.002 |
| 72 | 0.002 |
| 73 | 0.001 |
| 74 | 0.002 |
| 75 | 0.003 |
| 76 | 0.004 |
| 77 | 0.0006 |
| 78 | 0.006 |
| 79 | 0.006 |
| 80 | 0.002 |
| 81 | 0.011 |
| 82 | 0.002 |
| 83 | 0.005 |
| 84 | 0.005 |
| 85 | 0.003 |
| 86 | 0.003 |
| 87 | 0.002 |
| 88 | 0.0005 |
| 89 | 0.013 |
| 90 | 0.005 |
| 91 | 0.072 |
| 92 | 0.004 |
| 93 | 0.012 |
| 94 | 0.006 |
| 95 | 0.008 |
| 96 | 0.021 |
| 97 | 0.005 |
| 98 | 0.002 |
| 99 | 0.0006 |
| 100 | 0.0005 |
| 101 | 0.010 |
| 102 | 0.050 |
| 103 | 0.002 |
| 104 | 0.0007 |
| 105 | 0.004 |
| 106 | 0.001 |
| 107 | nd |
| 108 | 0.003 |
| 109 | 0.013 |
| 110 | 0.002 |
| 111 | 0.002 |
| 112 | 0.005 |
| 113 | 0.003 |
| 114 | 0.024 |
| 115 | 0.001 |
| 116 | 0.001 |
| 117 | 0.001 |
| 118 | 0.001 |
| 119 | 0.001 |
| 120 | 0.002 |
| 121 | 0.002 |
| 122 | 0.001 |
| 123 | 0.002 |
| 124 | 0.002 |
| 125 | 0.003 |
| 126 | 0.0004 |
| 127 | 0.004 |
| 128 | 0.002 |
| 129 | 0.003 |
| 130 | 0.013 |
| 131 | 0.010 |
| 132 | 0.007 |
| 133 | 0.004 |
| 134 | 0.007 |
| 135 | 0.003 |
| 136 | 0.002 |
| 137 | 0.053 |
| 138 | 0.071 |
| 139 | 0.027 |
| 140 | 0.053 |
| 141 | 0.008 |
| 142 | 0.006 |
| 143 | 0.004 |
| 144 | 0.078 |
| 145 | 0.005 |
| 146 | 0.142 |
| 147 | 0.328 |
| 148 | 0.007 |
| 149 | 0.006 |
| 150 | 0.012 |
| 151 | 0.006 |
| 152 | 0.060 |
| 153 | 0.029 |
| 154 | 0.052 |
| 155 | 0.011 |
| 156 | 0.001 |
| 157 | 0.022 |
| 158 | 0.016 |
| 159 | 0.002 |
| 160 | 0.013 |
| 161 | 0.009 |
| 162 | 0.007 |
| 163 | 0.006 |
| 164 | 0.004 |
| 165 | 0.008 |
| 166 | 0.011 |
| 167 | 0.005 |
| 168 | 0.004 |
| 169 | 0.007 |
| 170 | 0.019 |
| 171 | 0.005 |
| 172 | 0.014 |
| 173 | 0.021 |
| 174 | 0.011 |
| 175 | 0.006 |
| 176 | 0.005 |
| 177 | 0.012 |
| 178 | 0.002 |
| 179 | 0.005 |
| 180 | 0.003 |
| 181 | 0.006 |
| 182 | 0.027 |
| 183 | 0.006 |
| 184 | 0.008 |
| 185 | 0.011 |
| 186 | 0.037 |
| 187 | 0.107 |
| 188 | nd |
| 189 | 0.041 |
| 190 | 0.003 |
| 191 | 0.002 |
| 192 | 0.001 |
| 193 | 0.004 |
| 194 | 0.0006 |
| 195 | 0.001 |
| 196 | 0.019 |
| 197 | 0.011 |
| 198 | 0.012 |
| 199 | 0.004 |
| 200 | 0.002 |
| 201 | 0.001 |
| 202 | 0.075 |
| 203 | 0.007 |
| 204 | 0.001 |
| 205 | 0.006 |
| 206 | 0.002 |
| 207 | 0.036 |
| 208 | 0.035 |
| 209 | 0.004 |
| 210 | 0.002 |
| 211 | 0.003 |
| 212 | 0.0003 |
| 213 | 0.0006 |
| 214 | 0.009 |
| 215 | nd |
| 216 | nd |
| 217 | 0.010 |
| 218 | 0.0002 |
| 219 | 0.002 |
| 220 | 0.002 |
| 221 | 0.0003 |
| 222 | 0.008 |
| 223 | 0.015 |
| 224 | nd |
| 225 | 0.001 |
| 226 | 0.005 |
| 227 | nd |
| 228 | 0.052 |
| 229 | 0.013 |
| 230 | 0.003 |
| 231 | 0.0006 |
| 232 | 0.006 |
| 233 | 0.002 |
| 234 | 0.004 |
| 235 | 0.002 |
| 236 | 0.002 |
| 237 | 0.003 |
| 238 | 0.002 |
| 239 | 0.003 |
| 240 | 0.001 |
| 241 | 0.007 |
| 242 | 0.005 |
| 243 | 0.007 |
| 244 | 0.003 |
| 245 | 0.002 |
| 246 | 0.0004 |
| 247 | 0.0008 |
| 248 | 0.001 |
| 249 | 0.002 |
| 250 | 0.002 |
| 251 | 0.002 |
| 252 | 0.001 |
| 253 | 0.001 |
| 254 | 0.0006 |
| 255 | 0.0008 |
| 256 | 0.0008 |
| 257 | 0.001 |
| 258 | 0.001 |
| 259 | 0.001 |
| 260 | 0.011 |
| 261 | 0.028 |
| 262 | 1.126 |
| 263 | 0.003 |
| 264 | 0.003 |
| 265 | 0.001 |
| 266 | 0.011 |
| 267 | 0.009 |
| 268 | 0.05 |
| 269 | 0.009 |
| 270 | 0.0006 |
| 271 | 0.003 |
| 272 | 0.002 |
| 273 | 0.004 |
| 274 | 0.016 |
| 275 | 0.008 |
| 276 | 0.014 |
| 277 | 0.006 |
| 278 | 0.004 |
| 279 | 0.006 |
| 280 | 0.02 |
| 281 | nd |
| 282 | nd |
| 283 | 0.003 |
| 284 | 0.003 |
| 285 | 0.0006 |
| 286 | 0.001 |
| 287 | 0.379 |
| 288 | 0.001 |
| 289 | 0.001 |
| 290 | 0.001 |
| 291 | 0.002 |
| 292 | 0.002 |
| 293 | 0.001 |
| 294 | 0.002 |
| 295 | 0.002 |
| 296 | 0.002 |
| 297 | 0.001 |
| 298 | 0.001 |
| 299 | 0.001 |
| 300 | 0.001 |
| 301 | 0.001 |
| 302 | 0.0006 |
| 303 | 0.0006 |
| 304 | 0.0005 |
| 305 | 0.001 |
| 306 | 0.0006 |
| 307 | 0.001 |
| 308 | 0.0007 |
| 309 | 0.0004 |
| 310 | 0.0005 |
| 311 | 0.001 |
| 312 | 0.001 |
| 313 | 0.001 |
| 314 | 0.005 |
| 315 | 0.002 |
| 316 | 0.002 |
| 317 | 0.001 |
| 318 | 0.003 |
| 319 | 0.007 |
| 320 | 0.002 |
| 321 | 0.0005 |
| 322 | 0.0007 |
| 323 | 0.001 |
| 324 | nd |
| 325 | 0.003 |
| 326 | 0.003 |
| 327 | 0.005 |
| 328 | nd |
| 329 | 0.0005 |
| 330 | 0.0004 |
| 331 | 0.0005 |
| 332 | 0.0005 |
| 333 | nd |
| 334 | 0.004 |
| 335 | nd |
| 336 | 0.002 |
| 337 | 0.002 |
| 338 | 0.001 |
| 339 | 0.002 |
| 340 | 0.002 |
| 341 | 0.003 |
| 342 | 0.002 |
| 343 | 0.002 |
| 344 | 0.002 |
| 345 | 0.002 |
| 346 | 0.005 |
| 347 | 0.001 |
| 348 | 0.0006 |
| 349 | 0.001 |
| 350 | 0.003 |
| 351 | 0.0008 |
| 352 | nd |
| 353 | 0.0008 |
| 354 | 0.0005 |
| 355 | 0.001 |
| 356 | nd |
| 357 | 0.0008 |
| 358 | 0.0012 |
| 359 | 0.002 |
| 360 | 0.0008 |
| 361 | 0.004 |
| 362 | 0.008 |
| 363 | 0.013 |
| 364 | 0.002 |
| 365 | 0.002 |
| 366 | 0.005 |
| 367 | 0.001 |
| 368 | 0.001 |
| 369 | 0.002 |
| 370 | 0.001 |
| 371 | 0.001 |
| 372 | 0.001 |
| 373 | 0.003 |
| 374 | 0.0003 |
| 375 | 0.008 |
| 376 | 0.005 |
| 377 | 0.004 |
| 378 | 0.002 |
| 379 | 0.002 |
| 380 | 0.002 |
| 381 | 0.003 |
| 382 | 0.005 |
| 383 | 0.003 |
| 384 | 0.009 |
| 385 | 0.003 |
| 386 | 0.006 |
| 387 | 4.000 |
| 388 | 0.003 |
| 389 | 0.003 |
| 390 | 0.002 |
| 391 | 0.004 |
| 392 | 0.001 |
| 393 | 0.002 |
| 394 | 0.003 |
| 395 | 0.002 |
| 396 | 0.002 |
| 397 | 0.002 |
| 398 | 0.002 |
| 399 | 0.002 |
| 400 | 0.006 |
| 401 | 0.002 |
| 402 | 0.003 |
| 403 | 0.002 |
| 404 | 0.003 |
| 405 | 0.004 |
| 406 | 0.004 |
| 407 | 0.004 |
| 408 | 0.002 |
| 409 | 0.001 |
| 410 | 0.006 |
| 411 | 0.020 |
| 412 | 0.004 |
| 413 | 0.016 |
| 414 | 0.003 |
| 415 | 0.002 |
| 416 | 0.0006 |
| 417 | 0.012 |
| 418 | 0.107 |
| 419 | 0.031 |
| 420 | 0.013 |
| 421 | 0.008 |
| 422 | 0.01 |
| 423 | 0.008 |
| 424 | 0.016 |
| 425 | 0.126 |
| 426 | 0.0003 |
| 427 | 0.002 |
| 428 | 0.007 |
| 429 | 0.219 |
| 430 | 0.016 |
| 431 | 0.032 |
| 432 | 0.032 |
| 433 | 0.025 |
| 434 | 0.013 |
| 435 | 0.501 |
| 436 | 0.007 |
| 437 | 0.009 |
| 438 | 0.014 |
| 439 | 0.015 |
| 440 | nd |
| 441 | 0.007 |
| 442 | 0.005 |

nd: Not determined

It will be evident to one skilled in the art that the present disclosure is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

1. A compound of Formula I, 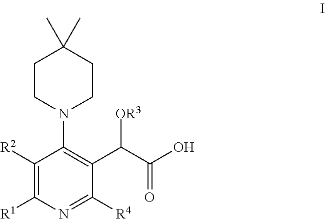
or a pharmaceutically acceptable salt thereof, wherein:
R¹ is hydrogen, halo, cyano, C₁₋₁₀alkyl, C₁₋₁₀haloalkyl, —C₁₋₁₀alkyl-OH, HO—C₁₋₁₀alkyl-O—, Ar¹, N(R⁵)(R⁶), —C(O)N(R⁷)(R⁸), or (R⁹)(R¹⁰)NC₁₋₁₀alkyl-;
provided R¹ and R⁴ are not both alkyl;
R² is benzodioxolyl, naphthalenyl, phenyl, pyrazinyl, pyridazinyl, pyridinyl, pyrimidinyl, quinolinyl, isoquinolinyl, tetrazinyl, or triazinyl, and is optionally substituted with 1-4 substituents selected from cyano, carbamoyl, carboxyl, halo, hydroxy, C₁₋₁₀alkyl, C₁₋₁₀haloalkyl, —N(R⁵)(R⁷), C₁₋₁₀alkyl-O—, Ar⁴, Ar⁴—C₁₋₁₀alkyl-O—, (R⁵)(Ar⁴—C₁₋₁₀alkyl)N—, Ar⁴—O—C₁₋₁₀alkyl-, or (Ar⁴)(R⁵)N—C₁₋₁₀alkyl-;
R³ is C₁₋₁₀alkyl;
R⁴ is hydrogen, cyano, halo, C₁₋₁₀haloalkyl, C₁₋₁₀alkyl, C₁₋₁₀alkyl-O—, C₁₋₁₀alkenyl, NH₂, hydroxy, —C₁₋₁₀alkyl-OH, carbamoyl, azetidinyl, pyrrolidinyl, piperidinyl, morpholinyl, or piperazinyl; provided R¹ and R⁴ are not both alkyl;
R⁵ is hydrogen, or C₁₋₁₀alkyl;
R⁶ is hydrogen, C₁₋₁₀alkyl, C₁₋₁₀alkyl-O—C₁₋₁₀alkyl-, C₁₋₁₀alkyl-O—C(O)—, C₃₋₉cycloalkyl, (C₃₋₉cycloalkyl)C₁₋₁₀alkyl-, 1-(C₁₋₁₀alkyl)piperidinyl-, tetrahydropyranyl, (tetrahydropyranyl)C₁₋₁₀alkyl-, morpholinoC₁₋₁₀alkyl-, (C₁₋₁₀alkyl)₂N—C₁₋₁₀alkyl-, piperidinylC₁₋₁₀alkyl-, 1-(C₁₋₁₀alkyl)piperidinylC₁₋₁₀alkyl-, 1-(C₁₋₁₀alkyl)piperazinylC₁₋₁₀alkyl-, Ar²—C₁₋₁₀alkyl-, Ar³, 1-(C₁₋₁₀alkylsulfonyl)piperidinyl-, or
1-(C₁₋₀alkylcarbonyl)piperidinyl-;
R⁷ is hydrogen, or C₁₋₁₀alkyl;
R⁸ is hydrogen, C₁₋₁₀alkyl, C₃₋₉cycloalkyl, (C₁₋₁₀alkyl)C₃₋₉cycloalkyl-, —SO₂(C₁₋₁₀alkyl), or —SO₂(C₃₋₉cycloalkyl);
R⁹ is hydrogen, or C₁₋₁₀alkyl;
R¹⁰ is hydrogen, C₁₋₁₀alkyl, (tetrahydropyranyl)C₁₋₁₀alkyl-, or C₁₋₁₀alkyl-O—C(O)—;
(R⁷)(R⁸)N taken together form an azetidinyl, pyrrolidinyl, piperidinyl, 1,1-dioxidothiomorpholinyl, or morpholinyl ring;
(R⁹)(R¹⁰)N taken together form an azetidinyl, azocanyl, pyrrolidinyl, piperidinyl, or azaspirononanyl ring, and is optionally substituted with 1-3 C₁₋₁₀alkyl substitutents;
Ar¹ is imidazolyl, pyrazolyl, pyridinyl, pyrimidinyl, pyrrolyl, or dihydrocyclopentapyrazolyl and is optionally substituted with 1-3 substitutents selected from amino, C₁₋₁₀alkyl, or C₃₋₉cycloalkyl;
Ar² is imidazolyl, pyrazolyl, or pyridinyl, and is optionally substituted with 1-3 substitutents selected from C₁₋₁₀alkyl and halo substitutents;
Ar³ is phenyl, pyridinyl, pyrazolyl, pyridazinyl, or pyrimidinyl, and is optionally substituted with 1-3 substituents selected from C₁₋₆alkyl, halo, carboxy, and cyano; and
Ar⁴ is phenyl, benzofuropyrimidinyl, or pyridofuropyrimidinyl and is optionally substituted with 1-3 substituents selected from cyano, halo, C₁₋₁₀alkyl, and C₁₋₁₀alkyl-O—;
and wherein each reference to “haloalkyl includes all halogenated isomers from monohalo to perhalo.

2. A compound or salt according to claim 1 wherein R¹ is hydrogen, halo, cyano, C₁₋₁₀alkyl, C₁₋₁₀haloalkyl, —C₁₋₁₀alkyl-OH, Ar¹, —N(R⁵)(R⁶), or (R⁹)(R¹⁰)NC₁₋₁₀alkyl-; provided R¹ and R⁴ are not both alkyl.

3. A compound or salt according to claim 2 wherein R¹ is hydrogen or (R⁹)(R¹⁰)NC₁₋₁₀alkyl-.

4. A compound or salt according to claim 1 wherein R² is phenyl, pyrazinyl, pyridazinyl, pyridinyl, pyrimidinyl, quinolinyl, or isoquinolinyl, and is optionally substituted with 1-4 substituents selected from cyano, carbamoyl, carboxyl, halo, hydroxy, C₁₋₁₀alkyl, C₁₋₁₀haloalkyl, —N(R⁵)(R′), C₁₋₁₀alkyl-O—, Ar⁴, Ar⁴—C₁₋₁₀alkyl-O—, (R⁵)(Ar⁴—C₁₋₁₀alkyl)N—, Ar⁴—O—C₁₋₁₀alkyl-, or (Ar⁴)(R⁵)N—C₁₋₁₀alkyl-.

5. A compound or salt according to claim 4 wherein R² is phenyl, pyrazinyl, pyridazinyl, pyridinyl, or pyrimidinyl, and is optionally substituted with 1-4 substituents selected from cyano, carbamoyl, carboxyl, halo, hydroxy, C₁₋₁₀alkyl, C₁₋₁₀haloalkyl, —N(R⁵)(R⁷), C₁₋₁₀alkyl-O—, Ar⁴, Ar⁴—C₁₋₁₀alkyl-O—, (R⁵)(Ar⁴—C₁₋₁₀alkyl)N—, Ar⁴—O—C₁₋₁₀alkyl-, or (Ar⁴)(R⁵)N—C₁₋₁₀alkyl-.

6. A compound or salt according to claim 1 wherein R⁴ is hydrogen, cyano, halo, C₁₋₁₀haloalkyl, C₁₋₁₀alkyl, C₁₋₁₀alkyl-O—, C₁₋₁₀alkenyl, hydroxy, or —C₁₋₁₀alkyl-OH; provided R¹ and R⁴ are not both alkyl. More preferably, R⁴ is C₁₋₁₀alkyl, cyano, halo, or C₁₋₁₀haloalkyl; provided R¹ and R⁴ are not both alkyl.

7. A compound or salt according to claim 1 wherein R¹ is hydrogen, R² is pyridinyl and is optionally substituted with a C₄alkyl-O—, R³ is C₄alkyl, and R⁴ is methyl.

8. The compound 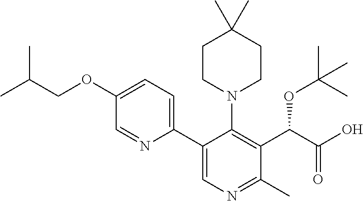 [SMILES: Cc1ncc(-c2ccc(OCC(C)C)cn2)c(N2CCC(C)(C)CC2)c1[C@H](OC(C)(C)C)C(=O)O]
or a pharmaceutically acceptable salt thereof.

9. The compound 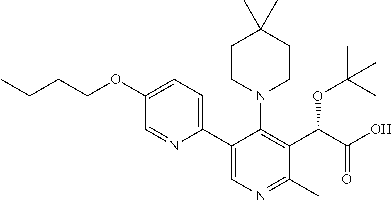 [SMILES: CCCCOc1ccc(-c2cnc(C)c([C@H](OC(C)(C)C)C(=O)O)c2N2CCC(C)(C)CC2)nc1]
or a pharmaceutically acceptable salt thereof.

10. A pharmaceutical composition comprising a compound or salt according to claim 1.

11. The composition of claim 10 further comprising at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, CAPSID inhibitors, HIV budding or maturation inhibitors, and HIV integrase inhibitors, and a pharmaceutically acceptable carrier.

12. A method for treating HIV infection comprising administering a composition according to claim 10 to a patient in need thereof.

13. The method of claim 12 further comprising administering at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, CAPSID inhibitors, HIV budding or maturation inhibitors, and HIV integrase inhibitors.

14-16. (canceled)
